(19) United States
(12) Patent Application Publication
(10) Pub. No.: US 2023/0135471 A1
(43) Pub. Date: May 4, 2023

(54) UBIQUITIN VARIANTS WITH IMPROVED AFFINITY FOR 53BP1

(71) Applicant: Integrated DNA Technologies, Inc., Coralville, IA (US)

(72) Inventors: Christopher Anthony Vakulskas, North Liberty, IA (US); Nicole Mary Bode, Oxford, IA (US); Steve Ehren Glenn, North Liberty, IA (US); Liyang Zhang, North Liberty, IA (US)

(21) Appl. No.: 17/952,252

(22) Filed: Sep. 24, 2022

Related U.S. Application Data
(60) Provisional application No. 63/321,384, filed on Mar. 18, 2022.
(60) Provisional application No. 63/278,155, filed on Nov. 11, 2021.
(60) Provisional application No. 63/248,300, filed on Sep. 24, 2021.

(51) Int. Cl. C07K 14/47; C12N 9/22; C12N 15/63

(52) U.S. Cl. CPC C07K 14/4702; C12N 9/22; C12N 15/63

(57) ABSTRACT

The present invention pertains to ubiquitin polypeptide variants (Ubvs) having improved affinity for 53BP1 relative to 53 ubiquitin polypeptide or i53 ubiquitin polypeptide, wherein the resultant interaction between the Ubvs and 53BP1 promotes increased homology directed repair of DNA double-strand break sites. Methods of suppressing 53BP1 recruitment to DNA double-strand break sites, increasing homologous recombination, increasing gene targeting, and editing a gene in a cell using a CRISPR system are provided with the Ubvs. Compositions and kits of Ubvs are also provided.

### CROSS-REFERENCE TO RELATED APPLICATIONS

This application claims benefit of priority under 35 U.S.C. 119 to U.S. Provisional Patent Application Ser. No. 63/248,300, filed Sep. 24, 2021, U.S. Provisional Patent Application Ser. No. 63/278,155, filed Nov. 11, 2021, and U.S. Provisional Patent Application Ser. No. 63/321,384, filed Mar. 18, 2022, wherein each application is entitled “UBIQUITIN VARIANTS WITH IMPROVED AFFINITY FOR 53BP1,” the contents of each application are herein incorporated by reference in its entirety.

### SEQUENCE LISTING

The instant application contains a Sequence Listing which has been submitted in ASCII format via EFS-Web and is hereby incorporated by reference in its entirety. Said ASCII copy, created on ______, is named IDT01-021-US_ST25.xml, and is ______ bytes in size.

### FIELD OF THE INVENTION

This invention pertains to ubiquitin polypeptide variants with increased affinity for 53BP1 and improved efficacy for enhancing homology directed repair rates.

### BACKGROUND OF THE INVENTION

Double-strand breaks (DSBs) of DNA are predominantly repaired through two mechanisms, non-homologous end joining (NHEJ), in which broken ends are rejoined, often imprecisely, or homology directed repair (HDR), which typically involves a sister chromatid or homologous chromosome being used as a repair template. HDR is facilitated by the presence of a sister chromatid and there are cellular mechanisms in place biasing repair towards NHEJ during the G1 phase of the cell cycle [1]. A key determinant of repair pathway choice is 53BP1. 53BP1 was first described as a binding partner of the tumor suppressor gene p53 and was later shown to be a key protein in NHEJ [2]. 53BP1 rapidly accumulates at sites of double-strand breaks. In G1, 53BP1 recruits RIF1 and inhibits end resection [3, 4]. End resection is a critical step in repair pathway choice, as it is necessary for HDR and inhibits NHEJ [1]. By inhibiting end resection, 53BP1 biases repair towards NEHJ and consequently loss of 53BP1 results in increased HDR [5]. Targeted nucleases can be introduced into cells in conjunction with a DNA repair template with homology to a targeted cut site to facilitate precise genome editing via HDR[6]. A strong inhibitor of 53BP1 is therefore useful for precise genome editing.

The recruitment of 53BP1 to DSB sites is dependent upon both H4K20 methylation and H2AK15 ubiquitination. 53BP1 has tandem Tudor domains that have been shown to specifically bind mono and dimethylated H4K20 and H4K20 methylation was shown to be important for 53BP1 recruitment to double-strand breaks [7, 8]. Introducing D1521R, a mutation that disrupts the activity of the Tudor domain, impairs the ability of 53BP1 to form ionizing radiation-induced foci [9]. The minimal focus-forming region of 53BP1 consists of the Tudor domain flanked by an N-terminal oligomerization region and a C-terminal extension. Notably, 53BP1 accumulation at DSBs requires the E3 ubiquitin ligase RNF168, that mediates H2AK13 and H2AK15 ubiquitination [10]. The C-terminal extension was shown to contain a ubiquitination-dependent recruitment motif (UDR) that binds specifically to H2AK15ub and is required for 53BP1 recruitment to DSB sites [9].

Thus, the ubiquitin polypeptide (SEQ ID NO:1) and its interaction with 53BP1 influences the repair pathway choice for DSB sites.

Due to the affinity of 53BP1 for ubiquitinated H2A, a screen of ubiquitin polypeptide variants for interaction with 53BP1 was conducted recently by Canny et al. in which they discovered and modified a ubiquitin polypeptide variant with selective binding to 53BP1 that they named i53 (inhibitor of 53BP1; SEQ ID NO: 2) [11]. The top five hits from the ubiquitin polypeptide variant screen were A10, A11, C08, G08, and H04, with G08 having the highest affinity. In contrast to what might be expected, the interaction of 53BP1 with G08 did not require the UDR and the interaction was shown to be between G08 and the 53BP1 Tudor domain. To generate i53, G08 was modified by introducing an I44A mutation that disrupts a solvent exposed hydrophobic patch on ubiquitin that most ubiquitin binding proteins interact with [9, 12]. Notably, this mutation in the context of H2AKcl5ub(I44A) interferes with 53BP1 interaction with ubiquitinated H2A, yet does not interfere with the ability of i53 to enhance HDR, consistent with i53 enhancing HDR through interaction with the 53BP1 Tudor domain and not the UDR domain [9, 11]. Additionally, i53 was modified relative to G08 through the removal of the C-terminal di-glycine motif Introduction of i53, but not a 53BP1 binding deficient i53 variant DM (i53 P69L+L70V), into cells inhibited the formation ionizing radiation induced 53BP1 foci. Introduction of i53 via plasmid delivery, adeno-associated virus mediated gene delivery, or delivery of mRNA were all shown to improve the rates of HDR. Rates of HDR were improved with the introduction of i53 using both double-stranded DNA donors and using single-stranded DNA donors, which have been shown to use different HDR mechanisms [11, 13, 14].

The present disclosure pertains to ubiquitin polypeptide variants (Ubvs) with increased affinity for 53BP1 and improved efficacy for enhancing HDR rates, and in particular, candidate amino acid changes in i53 that improve its affinity for 53BP1. Methods to identify such variants from a population of mutagenized ubiquitin polypeptides are provided, as well as the identification of additional beneficial mutations at specific amino acid positions. Improving the rate of HDR allows for increased rates of successful genome editing using the CRISPR/Cas9 system or other targeted nucleases in conjunction with supplying a repair template to direct precise genome editing events.

### BRIEF SUMMARY OF THE INVENTION

In a first aspect, an isolated polypeptide comprising a ubiquitin polypeptide variant is provided. The isolated polypeptide comprises at least one member selected from one of the following groups:

SEQ ID NO:450, wherein X₁ is selected from M, H, Y, W, Q, T, F, S, R, I, and N; X₂ is selected from Q, L, I, and M; X₆ is selected from K and R; X₇ is selected from T, M, I, C, L, and V; X₉ is selected from T, I, S, E and V; X₁₂ is selected from T, M, and Y; X₁₃ is selected from I, F, H and P; X₁₄ is selected from T, E, D, H, and N; X₁₆ is selected from E, M, T, N, Y, D, and H; X₁₇ is selected from V and C; X₁₈ is selected from E, M, Y, L, H, F, W, S, Q, T, C, N, R, and D; X₁₉ is selected from P and K; X₂₀ is selected from S, D, N, C, A, and W; X₂₁ is selected from D and E; X₂₅ is selected from N, V, I, E, G, M, Q, D, A, L, R, S, K, T, C, and F; X₂₆ is selected from I, V, and L; X₂₈ is selected from A, E, Q, W, I, M, and D; X₂₉ is selected from K, M, L, R, Q, and H; X₃1 is selected from Q, C, F, W, H, Y, L, R, and M; X₃₂ is selected from D, A, E, and R; X₃₃ is selected from K, H, A, Q, S, V, L, E, M, T, I, F, C, Y, R, N, and W; X₃₄ is selected from E and T; X₃₈ is selected from P, L, C, F, I, V, Y, T, M, H, S, Q, A, W, N, and K; X₃₉ is selected from D, W, E, G, S, L, and Q; X₄₀ is selected from Q, E, and D; X₄₁ is selected from Q, Y, I, C, and V; X₄₂ is selected from R, W, F, H, Y, N, C, and S; X₄₄ is selected from I, A and T; X₄₆ is selected from A, Q, and G; X₄₈ is selected from K, T, M, I, Q, V, R, L, and N; X₄₉ is selected from Q, S, L, M, P, E V, A, D, I, C, G, and N; X₅₁ is selected from E and D; X₅₂ is selected from D and E; X₅₄ is selected from R, Y, M, T, H, F, N, Q, K, and C; X₅ₛ is selected from T and R; X₅₇ is selected from S, G, D, N, H, E, A, Q, M, R, and K; X₅ₛ is selected from D and S; X₆₀ is selected from N, E, and Q; X₆₁ is selected from I and L; X₆₂ is selected from Q, L, T, V, C, A, M, I and S; X₆₃ is selected from K, I, M, F, and V; X₆₄ is selected from E, D, and S; X₆₅ is selected from S, P, E, K, H, R, A, D, N, and Q; X₆₆ is selected from T, K, R, and E; X₆₇ is selected from L, H, K, R, S, M, C, Y, and T; X₆₈ is selected from H, M, Q, and E; X₆₉ is selected from L, P, R, A, G, C, F, M, and S; X₇₀ is selected from V, L, M, F, and C; X₇₃ is selected from L and M; and X₇₄ is selected from R, Q, V, L, M, C, I, T, E, and K, and combinations thereof, provided that SEQ ID NOS:1-3 are excluded; and

at least one member selected from the group of SEQ ID NOs:452-665.

In a second aspect, an isolated polypeptide comprising an isolated fusion polypeptide having an Ubv amino acid sequence with an N-terminal His₆-tag is provided. The isolated fusion polypeptide comprises at least one member selected from the following: an isolated fusion polypeptide comprising SEQ ID NO:1100, wherein X₁₂ is selected from M, H, Y, W, Q, T, F, S, R, I, and N; X₁₃ is selected from Q, L, I, and M; X₁₇ is selected from K and R; X₁₈ is selected from T, M, I, C, L, and V; X₂₀ is selected from T, I, S, E and V; X₂₃ is selected from T, M, and Y; X₂₄ is selected from I, F, H and P; X₂₅ is selected from T, E, D, H, and N; X₂₇ is selected from E, M, T, N, Y, D, and H; X₂₈ is selected from V and C; X₂₉ is selected from E, M, Y, L, H, F, W, S, Q, T, C, N, R, and D; X₃₀ is selected from P and K; X₃₁ is selected from S, D, N, C, A, and W; X₃₂ is selected from D and E; X₃₆ is selected from N, V, I, E, G, M, Q, D, A, L, R, S, K, T, C, and F; X₃₇ is selected from I, V, and L; X₃₉ is selected from A, E, Q, W, I, M, and D; X₄₀ is selected from K, M, L, R, Q, and H; X₄₂ is selected from Q, C, F, W, H, Y, L, R, and M; X₄₃ is selected from D, A, E, and R; X₄₄ is selected from K, H, A, Q, S, V, L, E, M, T, I, F, C, Y, R, N, and W; X₄₅ is selected from E and T; X₄₉ is selected from P, L, C, F, I, V, Y, T, M, H, S, Q, A, W, N, and K; X₅₀ is selected from D, W, E, G, S, L, and Q; X₅₁ is selected from Q, E, and D; X₅₂ is selected from Q, Y, I, C, and V; X₅₃ is selected from R, W, F, H, Y, N, C, and S; X₅ₛ is selected from I, A and T; X₅₇ is selected from A, Q, and G; X₅₉ is selected from K, T, M, I, Q, V, R, L, and N; X₆₀ is selected from Q, S, L, M, P, E V, A, D, I, C, G, and N; X₆₂ is selected from E and D; X₆₃ is selected from D and E; X₆₅ is selected from R, Y, M, T, H, F, N, Q, K, and C; X₆₆ is selected from T and R; X₆₈ is selected from S, G, D, N, H, E, A, Q, M, R, and K; X₆₉ is selected from D and S; X₇₁ is selected from N, E, and Q; X₇₂ is selected from I and L; X₇₃ is selected from Q, L, T, V, C, A, M, I and S; X₇₄ is selected from K, I, M, F, and V; X₇₅ is selected from E, D, and S; X₇₆ is selected from S, P, E, K, H, R, A, D, N, and Q; X₇₇ is selected from T, K, R, and E; X₇₈ is selected from L, H, K, R, S, M, C, Y, and T; X₇₉ is selected from H, M, Q, and E; X₈₀ is selected from L, P, R, A, G, C, F, M, and S; X₈₁ is selected from V, L, M, F, and C; X₈₄ is selected from L and M; and X₈₅ is selected from R, Q, V, L, M, C, I, T, E, and K, and combinations thereof, provided that SEQ ID NO: 3 is excluded; and an isolated fusion polypeptide comprising at least one member selected SEQ ID NOS:235-244 and 246-449.

In a third aspect, an isolated polypeptide that enhances HDR activity through interactions with 53BP1 in a manner to influence repair mechanisms at DSB sites is provided. The isolated polypeptide includes a Ubv having at least 40% amino acid sequence identity to amino acid positions 1-74 of SEQ ID NOS:1, 2, 482, 633, or 450, provided that SEQ ID NOS:1 and 2 are excluded, and those having at least 40% amino acid sequence identity with amino acid positions 12-85 of SEQ ID NOS: 3, 241, 417, or 1100, provided that SEQ ID NO:3 is excluded. The isolated polypeptide provides enhanced HDR activity through interactions with 53BP1 in a manner to influence repair mechanisms at DSB sites relative to SEQ ID NO:1 under identical conditions.

In a fourth aspect, an isolated polynucleotide is provided. The isolated polynucleotide encodes the isolated polypeptide of any of the first, second, or third aspects.

In a fifth aspect, an isolated polynucleotide encoding a ubiquitin polypeptide variant is provided. The isolated polynucleotide comprises at least one member selected from SEQ ID NOS:669-682, 885-890, and 892-1099, and the corresponding RNA counterparts thereof.

In a sixth aspect, a vector comprising an isolated polynucleotide encoding a ubiquitin polypeptide variant is provided. The isolated polynucleotide comprises at least one member selected from SEQ ID NOS:669-682, 885-890, and 892-1099, and the corresponding RNA counterparts thereof.

In a seventh aspect, a cell or cell line comprising the isolated polypeptide of the first, second, or third aspects, the isolated polynucleotide of the fourth or fifth aspects, or the vector of the sixth aspect.

In an eighth aspect, a method of suppressing 53BP1 recruitment to DNA double-strand break sites in a cell is provided. The method includes a step of administering to the cell the isolated polypeptide of the first, second, or third aspects, the isolated polynucleotide of the fourth or fifth aspects, or the vector of the sixth aspect.

In a ninth aspect, a method of increasing homology-directed repair in a cell is provided. The method includes a step of administering to the cell the isolated polypeptide of the first, second, or third aspects, the isolated polynucleotide of the fourth or fifth aspects, or the vector of the sixth aspect.

In a tenth aspect, a method of editing a gene in a cell using a CRISPR system is provided. The method includes a step of administering to the cell the isolated polypeptide of the first, second, or third aspects, the isolated polynucleotide of the fourth or fifth aspects, or the vector of the sixth aspect.

In an eleventh aspect, a method of gene targeting in a cell is provided. The method includes a step of administering to the cell the isolated polypeptide of the first, second, or third aspects, the isolated polynucleotide of the fourth or fifth aspects, or the vector of the sixth aspect.

In a twelfth aspect, a composition comprising the isolated polypeptide the isolated polypeptide of the first, second or third aspects is provided.

In an thirteenth aspect, a kit comprising the isolated polypeptide of the first, second, or third aspects, the isolated polynucleotide of the fourth or fifth aspects, or the vector of the sixth aspect.

In a fourteenth aspect, a method of performing a medically therapeutic procedure is provided. The includes the step of performing genome editing according to any of the tenth or eleventh aspects.

In a fifteenth aspect, a method of screening for amino acid changes in a first polypeptide that improve affinity of the first polypeptide for a second polypeptide is provided. The method includes a step of using the BACTH system with a reporter gene under control of cAMP regulated promoter to allow fluorescence activated cell sorting based on protein-protein interaction affinity between the first polypeptide and the second polypeptide to screen for improved affinity variants of the first polypeptide.

### BRIEF DESCRIPTION OF THE DRAWINGS

FIG. 1 depicts exemplary reporter gene expression being dependent on the Ubv expressed as part of the two-hybrid system. The graphs show gating and distribution of reporter signal versus forward scatter for cells grown under moderate selection pressure expressing the 53BP1-two-hybrid component fusion protein along with i53, i53 53BP1-binding-deficient mutant (DM), or i53+K33A fusion proteins (K33A was identified as beneficial from our screen).

FIG. 2 depicts exemplary studies showing enrichment of individual amino acid changes had high correlation between experiments. The graph shows the average enrichment of individual amino acid (a.a.) changes between two experiments with different levels of selection pressure. Testing of i53 in the context of the two-hybrid screen resulted in ˜17% and ˜3% GFP positive population in the low and high selection pressure experiments, respectively. Error bars indicate standard deviation between two replicates for each experiment. The data shown is only for the 1010 a.a. changes for which there was at least 30 reads in the input for both replicates for both experiments.

FIG. 3 depicts exemplary studies showing positive enrichment values from the high-throughput screen correlate well with an increased two-hybrid reporter positive population when amino acid changes are screened individually. The graph shows the percent of reporter positive cells containing the Ubv fusion protein plasmid with the indicated amino acid change compared to the average enrichment measured from the low selection pressure screen. Vertical error bars indicate standard deviation from three biological replicates. Horizontal error bars indicate standard deviation from two biological replicates. Asterisks indicate a significant increase in the percentage of reporter positive cells with the indicated amino acid change relative to i53 (p<0.05, Dunnett's multiple comparisons test). The pooled screen enrichment indicated for i53 is for the unmodified plasmid relative to the pool of synonymous changes.

FIG. 4 depicts exemplary graphical data showing that Ubvs containing mutations identified by the two-hybrid screen in E. coli have improved in vitro affinity for the 53BP1 fragment. The graph plots the percent reporter positive cells expressing a fusion protein of the indicated Ubv plus a protein fragment used for the two hybrid system versus the affinity of purified i53 for a fragment of 53BP1 (Table 2) measured by BLI. The percentage of cells that are positive for reporter expression is an indication of the strength of the interaction in the two-hybrid screen. Ubvs consist of the i53 sequence plus the indicated amino acid substitutions or with no substitutions (i53). For the two-hybrid screen, Ubvs were tested individually and the data indicate the average of three replicates. The line is a simple linear regression of the data plotted in Prism with the R² value indicated.

FIG. 5A depicts an exemplary graph showing the association constant (1/dissociation constant) values measured in vitro using BLI of Ubvs proteins purified from E. coli (Table 3). The values are those calculated from the Kon and Kdis calculated from the 1:1 model fit of the protein association and dissociation (Table 4)

FIG. 5B depicts an exemplary graph showing the measured BLI response (Table 4) for i53, CM1, and CM7 interaction with the 53BP1 fragment (Table 3). The response curve was plotted using Prism using a one site-specific binding nonlinear fit model with the calculated dissociation constant (K_{d}) and R² indicated.

FIG. 5C depicts exemplary graphs showing BLI response vs time for the association and dissociation steps (non-red colored lines) for the data used for part B, with the calculated model fit indicated by the red lines. The top line for each graph is for the association using 20.5 μM of the 53BP1 fragment, with each line below indicating the response with a decreasing amount of 53BP1 down to 0.0102 μM (see Table 4).

FIG. 5D shows the sequences of human ubiquitin compared to i53, CM1, and CM7. The blue highlighting indicates the amino acid changes identified in the original i53 publication as part of G08. The green highlighting indicates the amino acid changes in the CM1 and CM7 ubiquitin variants. The red highlighting indicates that I44A mutation of i53 that is thought to disrupt interaction with ubiquitin binding proteins other than 53BP1.

FIG. 6 depicts an exemplary graph showing the rate of perfect HDR (introduction of a 6 nucleotide sequence at a Cas9 cut site in SERPINC1) measured by NGS in response to increasing amounts of Ubvs used during nucleofection in HEK293 cells. The dotted line indicates the level of HDR with no Ubv added.

FIG. 7 depicts a majority of tested high enrichment score amino acid changes from the two-hybrid screen resulted in improved affinity for 53BP1 when added to i53. The graph shows fold change in affinity measured by BLI of Ubvs that have a single mutation identified from the two-hybrid screen added to the i53 sequence.

FIG. 8 depicts nine mutations in CM1 relative to i53 contribute to the affinity of binding to 53BP1. The graph shows the fold change in affinity measured by BLI of Ubvs that lack the indicated mutation relative to CM1 (Table 6).

FIG. 9A depicts identification of ubiquitin variants with improved affinity over CM1. The graph shows the fold change in affinity for 53BP1 measured by BLI of ubiquitin variants that possess single amino acid substitutions added to CM1.

FIG. 9B shows the fold change in affinity of ubiquitin variants for 53BP1 measured by BLI that possess multiple mutations added simultaneously to the mutations in CM1.

FIG. 9C shows the fold change in affinity of ubiquitin variants that have groups of mutations identified or modified from those listed in FIG. 9B. added to CM1 simultaneously.

FIG. 9D shows the mutations present in the variants in FIGS. 9B and 9C relative to the sequence of i53 (SEQ ID NO: 2).

FIG. 10A shows higher affinity variants with additional stacked mutations better tolerate the introduction of 53BP1 binding deficient mutations. The graph shows the affinity (association constant Kₐ) of ubiquitin variants with and without the DM mutations (P69L, L70V). The sequences for the variants can be found in Table 6 (CM1-DM=CM107, CM138-DM=CM199, CM142-DM=CM203, CM143-DM=CM204, CM147-DM=CM208, CM149-DM=CM210, and CM158-DM=CM211)

FIG. 10B shows the rate of HDR (introduction of a 6 nucleotide sequence at a Cas9 cut site in SERPINC1) measured by EcoR1 cleavage of DNA PCR amplified from genomic DNA in response to increasing amounts of Ubvs used during nucleofection in HEK293 cells. The dashed line indicates the level of HDR with no Ubv added.

FIG. 11A shows screening of positions 69 and 70 mutations that allow for high affinity ubiquitin variants containing none of the published i53 mutations. The graphs show the fold change in affinity for amino acid changes at position 69 or 70 introduced into CM142 DM (CM203).

FIG. 11B shows the affinity for a fragment of 53BP1 of ubiquitin variants containing combinations of mutations at positions 69 and 70 with CM476 as the base construct. CM476 is a derivative of CM142 DM (CM203) with the remaining unchanged i53 mutation positions (2, 62, 64, and 66) mutated to the amino acid with the second best enrichment score from the two-hybrid screen.

FIG. 11C shows the fold change in affinity of variants containing mutations at position 62 relative to the base construct (CM429) containing a proline at position 62.

FIG. 11D shows a comparison of the affinity of i53, CM7, CM1, and CM455 measured by BLI.

FIG. 11E illustrates the sequence comparison of the proteins in FIG. 11D.

FIG. 11F shows the rate of perfect HDR (introduction of a 6 nucleotide sequence at a Cas9 cut site in SERPINC1) measured by NGS in response to increasing amounts of Ubvs used during nucleofection in HEK293 cells. The dashed line indicates the level of HDR with no Ubv added. The data shown is for two replicates with a line connecting the means.

FIG. 12A illustrates use of a ubiquitin variant with high affinity for 53BP1 provides an additional benefit to HDR over the use of a DNA-PK inhibitor alone. The graph shows the rate of HDR (introduction of 729 bp coding sequence for GFP at a Cas9 cut site in CLTA, Table 7) measured by Oxford Nanopore Technology (ONT) sequencing using Cas9 RNP delivered by nucleofection with 37.5 i53 or CM1 and/or IDT Enhancers (IDT-E or Alt_R HDR Enhancer) as an HDR enhancer in K562 cells. Ubiquitin variants were delivered alongside 2 μM Cas9 RNP at 37.5 μM final concentration. IDT-E was added to media post nucleofection for 24 hours at 1 μM final dose. Double stranded DNA donor with 200 bp homology arms was delivered at 1.5 μg per nucleofection.

FIG. 12B shows the rate of HDR (introduction of a 6 nucleotide sequence at a Cas9 cut site in MET) measured by EcoR1 cleavage of DNA PCR amplified from genomic DNA from HEK293 cells edited with Cas9 RNP targeting MET (Table 7) using Lonza nucleofection with either 12.5 μM CM1 co-delivered with 2 μM Cas9 RNP and/or treatment with 1 μM IDT-E for 24 hours with 1 μM Alt-R HDR donor oligo (Table 7).

FIG. 13 depicts screening of amino acid changes at position 2 of CM455 (SEQ ID NO:633) identified a more beneficial amino acid change. The graph shows the fold change in affinity for ubiquitin variants (CM489 (SEQ ID NO:658), CM455 (SEQ ID NO:633), CM478 (SEQ ID NO:647), CM479 (SEQ ID NO:648), CM480 (SEQ ID NO:649), CM481 (SEQ ID NO:650), CM483 (SEQ ID NO:652), CM485 (SEQ ID NO:654), CM486 (SEQ ID NO:655), CM487 (SEQ ID NO:656), CM488 (SEQ ID NO:657), CM490 (SEQ ID NO:659), CM491 (SEQ ID NO:660), CM492 (SEQ ID NO:661), CM493 (SEQ ID NO:662), CM494 (SEQ ID NO:663), CM495 (SEQ ID NO:664), and CM496 (SEQ ID NO:665)) containing a mutation at position 2 (relative to position 1 of WT ubiquitin (SEQ ID NO: 1)) of CM455 (SEQ ID NO:633). Fold change in affinity measured by BLI is shown relative CM489 (SEQ ID NO:658) which has a leucine at position 2.

FIG. 14 depicts a summary of amino acid sequences located in the wild-type human ubiquitin polypeptide (SEQ ID NO:1), i53 (SEQ ID NO:2), and the preferred ubiquitin polypeptide variant sequences (SEQ ID NO:450), wherein the preferred amino acid changes are listed below from top (highest) to bottom (lowest) average enrichment score from replicate experiments (see Examples). The dark grey background amino acids present in i53 that are not present in wildtype human ubiquitin. The non-underlined amino acid changes listed below the 3 reference sequences had a positive average enrichment score (average of two same day replicates) when added to i53 in at least one of two experiments. The single-underlined amino acid changes were identified as beneficial using BLI experiments in specific backgrounds (See Example 4 and Example 6). The double-underlined amino acid changes used in CM455 that were identified from the screen as having the highest enrichment score at that position (even if it was slightly negative). The light grey-shaded amino acid changes meet the same criteria as the non-underlined amino acids and were also described as potentially beneficial in the patent for i53 (SEQ ID NO:2) (WO2017132746A1. The black background shaded amino acid (i.e., position 67, K) is an amino acid change that meets the same criteria as the non-underlined amino acids but was also identified as potentially beneficial in the patent for i53 (SEQ ID NO:2) (see EP3411391 (B1) to Durocher et al.).

FIG. 15 demonstrates tag-free CM1 (CM1tf) is as active as His₆-tagged CM1 in boosting rates of HDR. The graph shows the percent HDR measured by EcoR1 cleavage assay with varying amounts of CM1 (His₆-tagged CM1; SEQ ID NO:241) or tag-free CM1 (CM1tf, SEQ ID NO:482). Cas9 RNP (2 μM) targeting HPRT1 (Table 7) was delivered with varying amounts of ubiquitin variant (50 μM to 1.56 μM in two fold increments) into cells by Lonza nucleofection along with 2 μM HDR donor (40 bp homology arms, 6 bp EcoR1 cut site insert). Data is shown for two biological replicates with lines connecting the means. The dashed line indicates the level of EcoR1 cleavage when no enhancer is used (n=3, standard deviation <2%).

FIG. 16A depicts a graph showing the rate of HDR measured by EcoR1 cleavage assay in HEK293 cells that constitutively express HiFi Cas9 when plasmid (154 ng) encoding Cas9 sgRNA targeting HPRT1 plus 2 μM ssDNA donor (Table 7) was introduced into cells by Lonza Nucleofection. Plasmid (154 ng) for expression of His-tagged i53, His-tagged CM1, or a crRNA for LbCas12a (negative control) was co-delivered with the sgRNA expression plasmid and ssDNA donor as indicated. Error bars indicate the standard deviation from two replicates.

FIG. 16B depicts a graph shows the rate of HDR measured by EcoR1 cleavage assay in Jurkat cells which had CM1tf delivered as either mRNA or protein. CM1tf protein or mRNA encoding CM1tf was delivered with 2 μM Cas9 RNP targeting HPRT1 and 2 μM ssDNA donor (Table 7) into Jurkat cells by Lonza nucleofection. Error bars indicate the standard deviation from three replicates.

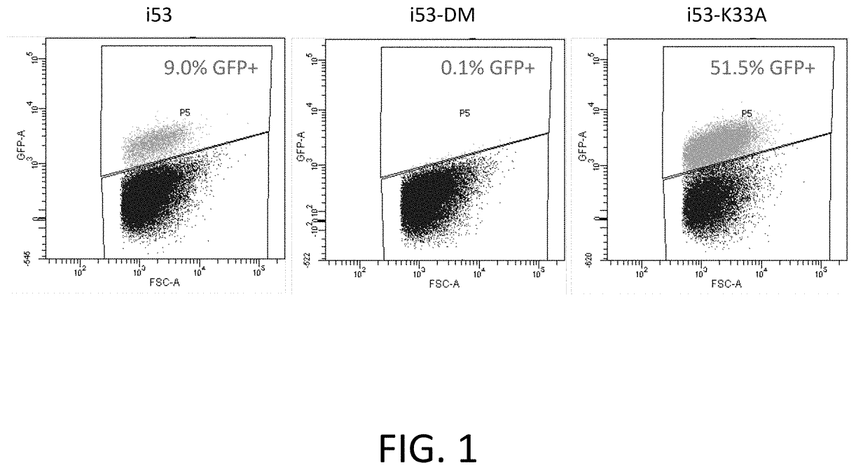

### DETAILED DESCRIPTION OF THE INVENTION

The current invention provides novel ubiquitin variants (Ubvs) with increased affinity for 53BP1 and improved efficacy for enhancing HDR rates. The identified Ubvs have increased affinity for 53BP1 and improved efficacy for enhancing HDR rates. Among the identified Ubvs include candidate amino acid changes in i53 that would improve its affinity for 53BP1 as well as Ubvs that do not include any of mutations present in the published i53 sequence. Methods to identify such variants from a population of mutagenized ubiquitin polypeptides are provided, as well as the identification of additional beneficial mutations at specific amino acid positions. Methods are provided that improve the rate of HDR and allow for increased rates of successful genome editing using the CRISPR/Cas9 system or other targeted nucleases in conjunction with supplying a repair template to direct precise genome editing events.

Screening methods to identify novel ubiquitin polypeptide variants

An initial filing identified ubiquitin variants (Ubvs) with increased affinity for 53BP1 and improved efficacy for enhancing HDR rates. In order to identify mutations that improve the affinity of i53 for 53BP1, a two-hybrid screen was conducted to identify variants with improved affinity. We engineered the screen such that interaction of two candidate proteins is tied to expression of a reporter gene that can be measured by fluorescence activated cell sorting (FACS). That disclosure described the results of a screen that interrogated the effect of all possible single amino acid substitutions individually at every position in i53 (a.a. 1-74) on the expression of a reporter gene in a two-hybrid assay in E. coli. From that screening method, about 230 amino acid changes were identified as candidates for improving the affinity of i53 for 53BP1. Of the 24 amino acid changes tested individually, 16 of them resulted in a statistically significant increase in percent of cells that were positive for reporter expression relative to i53. See Example 1 for details. See U.S. Provisional Patent Application Ser. No. 63/248,300, filed Sep. 24, 2021, and entitled “UBIQUITIN VARIANTS WITH IMPROVED AFFINITY FOR 53BP1” (Attorney Docket No. IDT01-021-PRO), the contents of which is incorporated by reference in its entirety.

A subsequent filing described the testing of a subset of those mutations individually and in combination for their effects on the affinity of the two proteins in vitro and on the ability to enhance HDR. From this testing, several individual mutations that change amino acids at the surface of i53 that interacts with 53BP1 were found to significantly improve the affinity of i53 for 53BP1. When mutations were combined together, the highest affinity Ubv (CM1) had a 50 to 100 fold improvement in the affinity for a fragment of 53BP1 relative to the published i53 sequence. Two of the Ubvs that contain multiple mutations relative to i53 were tested for their ability to improve HDR in HEK293 cells. These tests revealed that the improved affinity ubiquitin variants require about a 10 fold lower dose for maximum effectiveness and that HDR rates were improved beyond what could be achieved with the i53 peptide. See U.S. Provisional Patent Application Ser. No. 63/278,155, filed Nov. 11, 2021, and entitled “UBIQUITIN VARIANTS WITH IMPROVED AFFINITY FOR 53BP1” (Attorney Docket No. IDT01-021-PRO2), the contents of which is incorporated by reference in its entirety.

A subsequent filing evaluated additional individual mutations in the context of i53 and CM1 and identified novel combinations of mutations that further improve affinity beyond that of CM1. Additionally, novel beneficial mutations beyond those identified in the screen at specific amino acid positions were identified. Combining the novel beneficial mutations with screen identified mutations resulted in the generation of Ubvs that do not include any of the mutations present in the published i53 sequence and have dramatically improved affinity for 53BP1 compared to i53. See U.S. Provisional Patent Application Ser. No. 63/321,384, filed Mar. 18, 2022 and entitled “UBIQUITIN VARIANTS WITH IMPROVED AFFINITY FOR 53BP1” (Attorney Docket No. IDT01-021-PRO3), the contents of which is incorporated by reference in its entirety.

Using a combination of amino acid changes from the two-hybrid screen and identified through specific position screens (see Example 4), a ubiquitin variant (CM455) was identified that does not contain any of the mutations present in i53 yet maintains affinity comparable to CM1. Additional individual mutations in the context of CM455 at position 2 were evaluated and identified a novel mutation that that results in a variant (CM487) with improved affinity beyond that of CM455. (See Example 6).

### Isolated Ubiquitin Polypeptide Variants

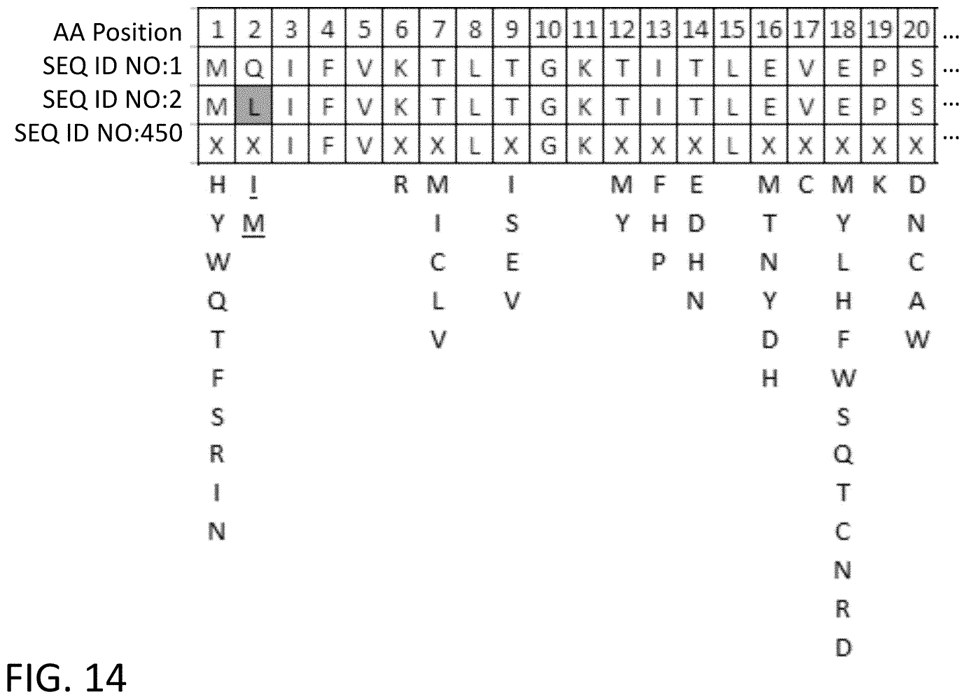

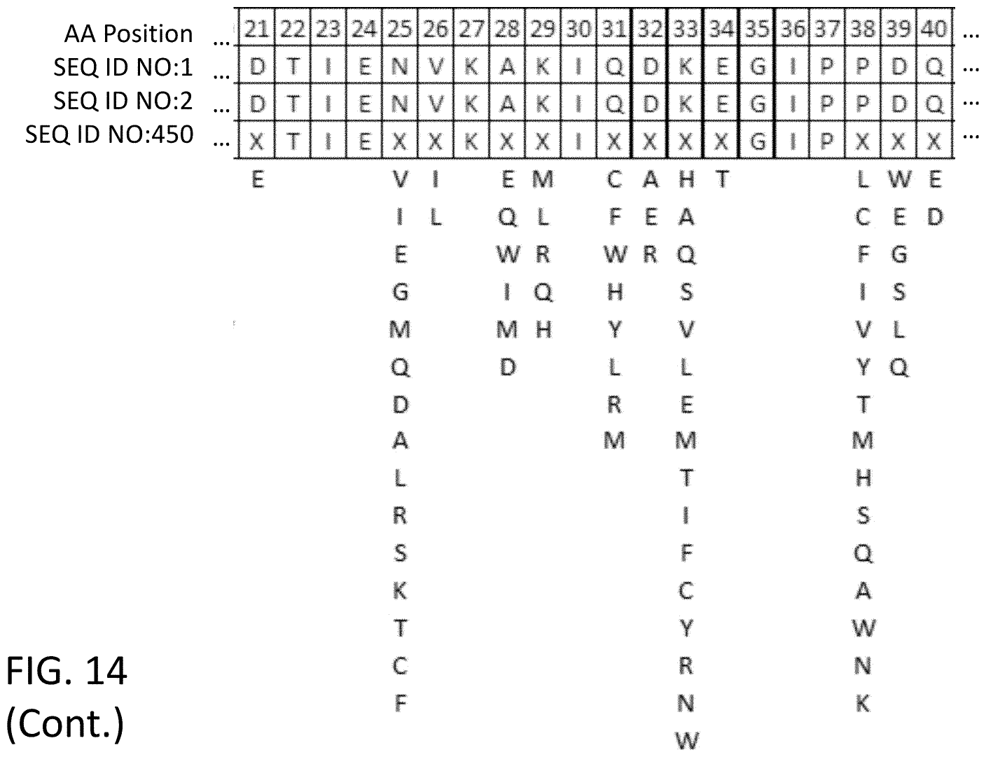

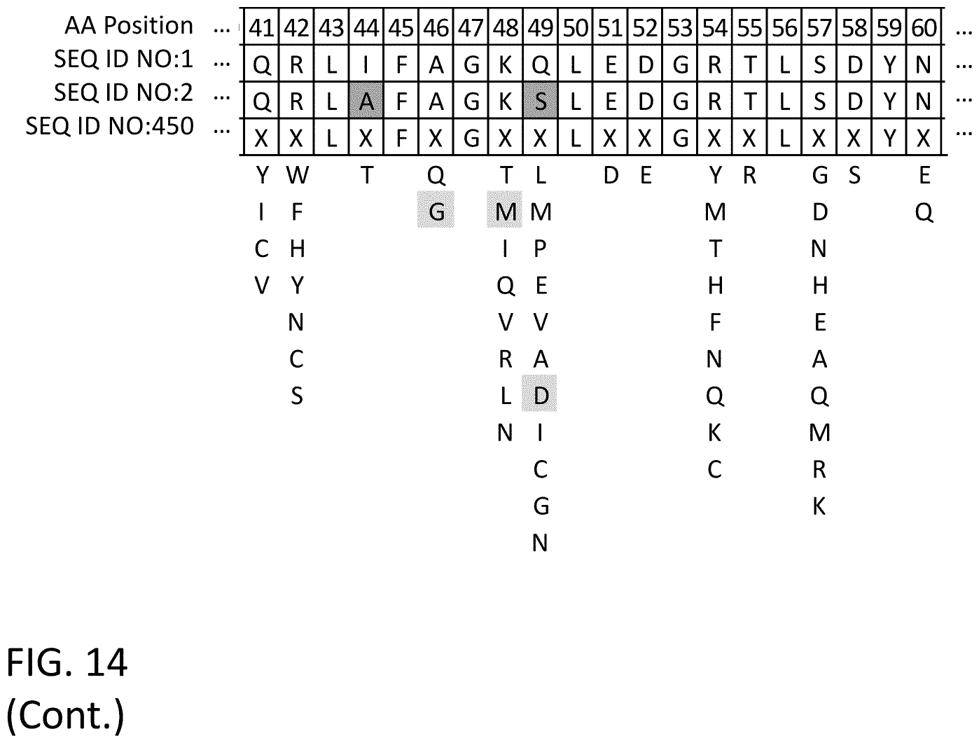

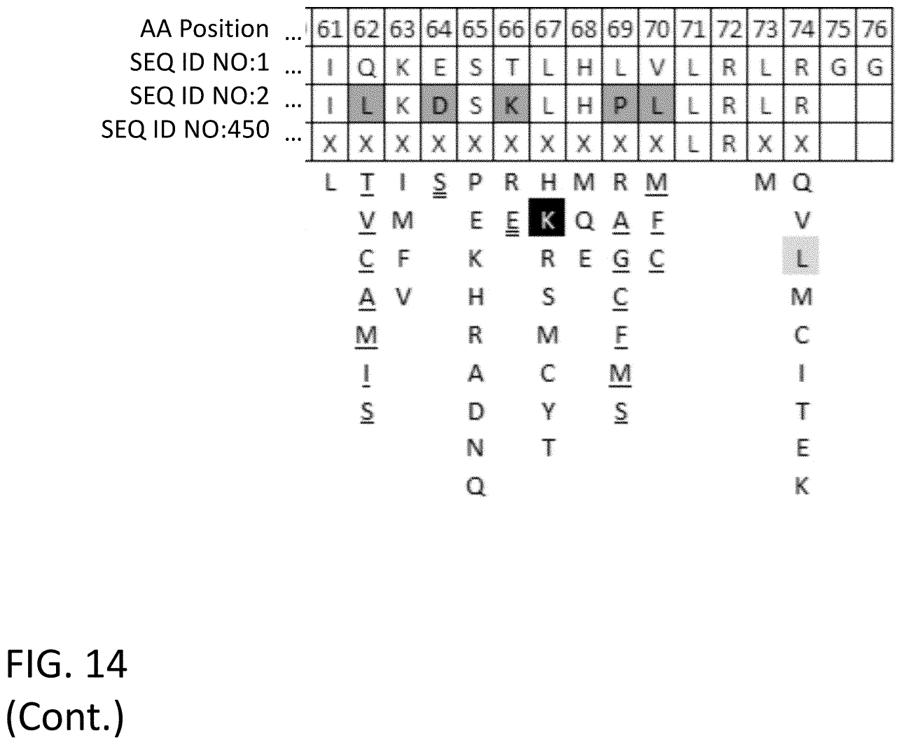

Referring to FIG. 14, preferred isolated Ubv amino acid sequences include those summarized by SEQ ID NO:450:

N-XXIFVXXLXG KXXXLXXXXX XTIEXXKXXI XXXXGIPXXX
XXLXFXGXXL XXGXXLXXYX XXXXXXXXXX LRXX-C

wherein X₁ is selected from M, H, Y, W, Q, T, F, S, R, I, and N; X₂ is selected from Q, L, I, and M; X₆ is selected from K and R; X₇ is selected from T, M, I, C, L, and V; X₉ is selected from T, I, S, E and V; X₁₂ is selected from T, M, and Y; X₁₃ is selected from I, F, H and P; X₁₄ is selected from T, E, D, H, and N; X₁₆ is selected from E, M, T, N, Y, D, and H; X₁₇ is selected from V and C; X₁₈ is selected from E, M, Y, L, H, F, W, S, Q, T, C, N, R, and D; X₁₉ is selected from P and K; X₂₀ is selected from S, D, N, C, A, and W; X₂₁ is selected from D and E; X₂₅ is selected from N, V, I, E, G, M, Q, D, A, L, R, S, K, T, C, and F; X₂₆ is selected from I, V, and L; X₂₈ is selected from A, E, Q, W, I, M, and D; X₂₉ is selected from K, M, L, R, Q, and H; X₃₁ is selected from Q, C, F, W, H, Y, L, R, and M; X₃₂ is selected from D, A, E, and R; X₃₃ is selected from K, H, A, Q, S, V, L, E, M, T, I, F, C, Y, R, N, and W; X₃₄ is selected from E and T; X₃₈ is selected from P, L, C, F, I, V, Y, T, M, H, S, Q, A, W, N, and K; X₃₉ is selected from D, W, E, G, S, L, and Q; X₄₀ is selected from Q, E, and D; X₄₁ is selected from Q, Y, I, C, and V; X₄₂ is selected from R, W, F, H, Y, N, C, and S; X₄₄ is selected from I, A and T; X₄₆ is selected from A, Q, and G; X₄₈ is selected from K, T, M, I, Q, V, R, L, and N; X₄₉ is selected from Q, S, L, M, P, E V, A, D, I, C, G, and N; X₅₁ is selected from E and D; X₅₂ is selected from D and E; X₅₄ is selected from R, Y, M, T, H, F, N, Q, K, and C; X₅₅ is selected from T and R; X₅₇ is selected from S, G, D, N, H, E, A, Q, M, R, and K; X₅₈ is selected from D and S; X₆₀ is selected from N, E, and Q; X₆₁ is selected from I and L; X₆₂ is selected from Q, L, T, V, C, A, M, I and S; X₆₃ is selected from K, I, M, F, and V; X₆₄ is selected from E, D, and S; X₆₅ is selected from S, P, E, K, H, R, A, D, N, and Q; X₆₆ is selected from T, K, R, and E; X₆₇ is selected from L, H, K, R, S, M, C, Y, and T; X₆₈ is selected from H, M, Q, and E; X₆₉ is selected from L, P, R, A, G, C, F, M, and S; X₇₀ is selected from V, L, M, F, and C; X₇₃ is selected from L and M; and X₇₄ is selected from R, Q, V, L, M, C, I, T, E, and K, and combinations thereof. These polypeptides of SEQ ID NO:450 are highly preferred, provided that polypeptides encoding SEQ ID NOS:1-3 are excluded.
Fusion Polypeptides with Ubvs Polypeptides Fused to Affinity Tag Motifs

Preferred Ubvs amino acid sequences include fusion polypeptides. Fusion polypeptides typically include extra amino acid information that is not native to the polypeptide to which the extra amino acid information is covalently attached. Such extra amino acid information may include tags that enable purification or identification of the fusion protein. Such extra amino acid information may also include peptides added to facilitate protein translation. Examples of such tags including adding an methionine or a methionine plus a short flexible linker (GGSG) (MGGSG; (SEQ ID NO:1113) to facilitate translation of protein variants where the X₁ is not M, such as in CM142 (SEQ ID NO: 557). Such extra amino acid information may include peptides that enable the fusion proteins to be transported into cells and/or transported to specific locations within cells such as peptides that act as nuclear localization signals. Examples of tags for these purposes include the following: AviTag, which is a peptide allowing biotinylation by the enzyme BirA so the protein can be isolated by streptavidin (GLNDIFEAQKIEWHE; SEQ ID NO:1114); Calmodulin-tag, which is a peptide bound by the protein calmodulin (KRRWKKNFIAVSAANRFKKISSSGAL; SEQ ID NO:1115); polyglutamate tag, which is a peptide binding efficiently to anion-exchange resin such as Mono-Q (EEEEEE; SEQ ID NO:1116); E-tag, which is a peptide recognized by an antibody (GAPVPYPDPLEPR; SEQ ID NO:1117); FLAG-tag, which is a peptide recognized by an antibody (DYKDDDDK; SEQ ID NO:1118); HA-tag, which is a peptide from hemagglutinin recognized by an antibody (YPYDVPDYA; SEQ ID NO:1119); His-tag, which is typically 5-10 histidines and can direct binding to a nickel or cobalt chelate (HHHHH; SEQ ID NO:1120); Myc-tag, which is a peptide derived from c-myc recognized by an antibody (EQKLISEEDL; SEQ ID NO:1121); NE-tag, which is a novel 18-amino-acid synthetic peptide (TKENPRSNQEESYDDNES; SEQ ID NO:1122) recognized by a monoclonal IgG1 antibody, which is useful in a wide spectrum of applications including Western blotting, ELISA, flow cytometry, immunocytochemistry, immunoprecipitation, and affinity purification of recombinant proteins; S-tag, which is a peptide derived from Ribonuclease A (KETAAAKFERQHMDS; SEQ ID NO:1123); SBP-tag, which is a peptide which binds to streptavidin; (MDEKTTGWRGGHVVEGLAGELEQLRARLEHHPQGQREP; SEQ ID NO:1124); Softag 1, which is intended for mammalian expression (SLAELLNAGLGGS; SEQ ID NO:1125); Softag 3, which is intended for prokaryotic expression (TQDPSRVG; SEQ ID NO:1126); Strep-tag, which is a peptide which binds to streptavidin or the modified streptavidin called streptactin (Strep-tag II: WSHPQFEK; SEQ ID NO:1127); TC tag, which is a tetracysteine tag that is recognized by FlAsH and ReAsH biarsenical compounds (CCPGCC; SEQ ID NO:1128) V5 tag, which is a peptide recognized by an antibody (GKPIPNPLLGLDST; SEQ ID NO:1129); VSV-tag, a peptide recognized by an antibody (YTDIEMNRLGK; SEQ ID NO:1130); Xpress tag (DLYDDDDK; SEQ ID NO:1131); Isopeptag, which is a peptide which binds covalently topilin-C protein (TDKDMTITFTNKKDAE; SEQ ID NO:1132); SpyTag, which is a peptide which binds covalently to SpyCatcher protein (AHIVMVDAYKPTK; SEQ ID NO:1133); and SnoopTag, a peptide which binds covalently to SnoopCatcher protein (KLGDIEFIKVNK; SEQ ID NO:1134).

An affinity tag can include flanking amino acids when the affinity tag is located at the N-terminus of the fusion polypeptide. Such flanking amino acids include an initiator methionine and flexible linker sequences.

A highly preferred affinity tag includes a His-tag (SEQ ID NO:1135). A highly preferred affinity tag includes an N-terminal His-tag (MHHHHHHGGSG; SEQ ID NO:1136). Highly preferred fusion polypeptides include Ubvs, such as SEQ ID NO: 3 fused to an N-terminal His-tag (e.g., SEQ ID NO:1136), as well as other preferred Ubvs amino acid sequences that include an N-terminal His-tag. A highly preferred translation tag includes N-terminal M (M) or M plus a short flexible linker (i.e., MGGSG: SEQ ID NO:1113).

A highly preferred fusion polypeptide of Ubvs comprises SEQ ID NO:1100:

N-MHHHHHHGGSG XXIFVXXLXG KXXXLXXXXX XTIEXXKXXI
XXXXGIPXXX XXLXFXGXXL XXGXXLXXYX XXXXXXXXXX
LRXX-C

wherein X₁₂ is selected from M, H, Y, W, Q, T, F, S, R, I, and N; X₁₃ is selected from Q, L, I, and M; X₁₇ is selected from K and R; X₁₈ is selected from T, M, I, C, L, and V; X₂₀ is selected from T, I, S, E and V; X₂₃ is selected from T, M, and Y; X₂₄ is selected from I, F, H and P; X₂₅ is selected from T, E, D, H, and N; X₂₇ is selected from E, M, T, N, Y, D, and H; X₂₈ is selected from V and C; X₂₉ is selected from E, M, Y, L, H, F, W, S, Q, T, C, N, R, and D; X₃₀ is selected from P and K; X₃₁ is selected from S, D, N, C, A, and W; X₃₂ is selected from D and E; X₃₆ is selected from N, V, I, E, G, M, Q, D, A, L, R, S, K, T, C, and F; X₃₇ is selected from I, V, and L; X₃₉ is selected from A, E, Q, W, I, M, and D; X₄₀ is selected from K, M, L, R, Q, and H; X₄₂ is selected from Q, C, F, W, H, Y, L, R, and M; X₄₃ is selected from D, A, E, and R; X₄₄ is selected from K, H, A, Q, S, V, L, E, M, T, I, F, C, Y, R, N, and W; X₄₅ is selected from E and T; X₄₉ is selected from P, L, C, F, I, V, Y, T, M, H, S, Q, A, W, N, and K; X₅₀ is selected from D, W, E, G, S, L, and Q; X₅₁ is selected from Q, E, and D; X₅₂ is selected from Q, Y, I, C, and V; X₅₃ is selected from R, W, F, H, Y, N, C, and S; X₅₅ is selected from I, A and T; X₅₇ is selected from A, Q, and G; X₅₉ is selected from K, T, M, I, Q, V, R, L, and N; X₆₀ is selected from Q, S, L, M, P, E V, A, D, I, C, G, and N; X₆₂ is selected from E and D; X₆₃ is selected from D and E; X₆₅ is selected from R, Y, M, T, H, F, N, Q, K, and C; X₆₆ is selected from T and R; X₆₈ is selected from S, G, D, N, H, E, A, Q, M, R, and K; X₆₉ is selected from D and S; X₇₁ is selected from N, E, and Q; X₇₂ is selected from I and L; X₇₃ is selected from Q, L, T, V, C, A, M, I and S; X₇₄ is selected from K, I, M, F, and V; X₇₅ is selected from E, D, and S; X₇₆ is selected from S, P, E, K, H, R, A, D, N, and Q; X₇₇ is selected from T, K, R, and E; X₇₈ is selected from L, H, K, R, S, M, C, Y, and T; X₇₉ is selected from H, M, Q, and E; X₈₀ is selected from L, P, R, A, G, C, F, M, and S; X₈₁ is selected from V, L, M, F, and C; X₈₄ is selected from L and M; and X₈₅ is selected from R, Q, V, L, M, C, I, T, E, and K, and combinations thereof, provided that SEQ ID NO: 3 is excluded.

Additional preferred fusion polypeptides of Ubvs include SEQ ID NOS:235-244 and 246-449.

### Preferred Isolated Ubv Polypeptides Include Those Having Significant Amino Acid Sequence Identity to Reference Sequences.

An isolated polypeptide that enhances rates of HDR through interactions with 53BP1 in a manner to influence repair mechanisms at DSB sites is provided. The isolated polypeptide comprises a Ubv having at least 40% amino acid sequence identity to amino acid positions 1-74 of SEQ ID NOS:1, 2, 482, 633, or 450, provided that SEQ ID NOS:1 and 2 are excluded, and those having at least 40% amino acid sequence identity with amino acid positions 12-85 of SEQ ID NOS: 3, 241, 417, or 1100, provided that SEQ ID NO:3 is excluded. Such an isolated polypeptide provides enhanced HDR activity through interactions with 53BP1 in a manner to influence repair mechanisms at DSB sites relative to SEQ ID NO:1 under identical conditions.

Preferred isolated polypeptides include those having amino acid sequence identity in the range of at least 50% to 100% identity with amino acid positions 1-74 of SEQ ID NOS:1, 2, 482, 633, or 450, provided that SEQ ID NOS:1 and 2 are excluded, and those having amino acid sequence identity in the range of at least 50% to 100% identity with amino acid positions 12-85 of SEQ ID NOS: 3, 241, 417, or 1100, provided that SEQ ID NO:3 is excluded. Even more preferably, preferred isolated polypeptides include those having amino acid sequence identity in the range of at least 60% to 100% identity with amino acid positions 1-74 of SEQ ID NOS:1, 2, 482, 633, or 450, provided that SEQ ID NOS:1 and 2 are excluded, and those having amino acid sequence identity in the range of at least 60% to 100% identity with amino acid positions 12-85 of SEQ ID NOS: 3, 241, 417, or 1100, provided that SEQ ID NO:3 is excluded. Even more preferably, preferred isolated polypeptides include those having amino acid sequence identity in the range of at least 70% to 100% identity with amino acid positions 1-74 of SEQ ID NOS:1, 2, 482, 633, or 450, provided that SEQ ID NOS:1 and 2 are excluded, and those having amino acid sequence identity in the range of at least 70% to 100% identity with amino acid positions 12-85 of SEQ ID NOS: 3, 241, 417, or 1100, provided that SEQ ID NO:3 is excluded. Even more preferably, preferred isolated polypeptides include those having amino acid sequence identity in the range of at least 80% to 100% identity with amino acid positions 1-74 of SEQ ID NOS:1, 2, 482, 633, or 450, provided that SEQ ID NOS:1 and 2 are excluded, and those having amino acid sequence identity in the range of at least 80% to 100% identity with amino acid positions 12-85 of SEQ ID NOS: 3, 241, 417, or 1100, provided that SEQ ID NO:3 is excluded. Even more preferably, preferred isolated polypeptides include those having amino acid sequence identity in the range of at least 90% to 100% identity with amino acid positions 1-74 of SEQ ID NOS:1, 2, 482, 633, or 450, provided that SEQ ID NOS:1 and 2 are excluded, and those having amino acid sequence identity in the range of at least 90% to 100% identity with amino acid positions 12-85 of SEQ ID NOS: 3, 241, 417, or 1100, provided that SEQ ID NO:3 is excluded. Even more preferably, preferred isolated polypeptides include those having amino acid sequence identity in the range of at least 95% to 100% identity with amino acid positions 1-74 of SEQ ID NOS:1, 2, 482, 633, or 450, provided that SEQ ID NOS:1 and 2 are excluded, and those having amino acid sequence identity in the range of at least 95% to 100% identity with amino acid positions 12-85 of SEQ ID NOS: 3, 241, 417, or 1100, provided that SEQ ID NO:3 is excluded.

A preferred polypeptide sequence in the aforementioned ranges with amino acid positions 1-74 of SEQ ID NOS:1, 2, 482, 633, or 450, provided that SEQ ID NOS:1 and 2 are excluded, and with amino acid positions 12-85 of SEQ ID NOS: 3, 241, 417, or 1100, provided that SEQ ID NO:3 is excluded, further provide a functional benefit of enhanced HDR rates when compared to HDR rates achieved when introducing human ubiquitin SEQ ID NO:1 into cells under identical conditions.

### Evaluation of Isolated Polypeptides Having a Functional Benefit of Enhanced HDR Rates

A preferred isolated polynucleotide encoding such isolated polypeptides within the stated ranges of % amino acid sequence identity to the aforementioned reference polypeptide sequence(s) in the aforementioned ranges, further provide a functional benefit of enhanced HDR rates when compared to HDR achieved when introducing human ubiquitin SEQ ID NO:1 into cells under identical conditions. Such enhanced HDR rates can be readily assessed by one of skill in the art based upon the teachings disclosed herein, including tests for at least one of the following functional properties: (1) a higher Kₐ (lower Kd) for binding a fragment of 53BP1 (amino acids 1484-1603) (See, for example, SEQ ID NO: 245) than is measured for Human ubiquitin (SEQ ID NO:1) under identical conditions as measured in vitro using BLI, even more preferably a higher measured Kₐ (lower K_{d}) for binding a fragment of 53BP1 (amino acids 1484-1603) (See SEQ ID NO: 245) than is measured for i53 (SEQ ID NO:2) under identical conditions as measured in vitro using BLI; (2) Delivery of the polypeptide in the form of mRNA, plasmid, or protein, results in improved HDR rates for introduction an EcoR1 cut site insert at the HPRT1 or SERPINC1 cut sites as specified by the sgRNA and ssDNA donor sequences in Table 7 as compared to delivery of human ubiquitin (SEQ ID NO: 1) under the same conditions. See Examples 3, 4, 7, and 8 for details.

### Isolated Nucleic Acids

Isolated nucleic acids encoding preferred Ubvs amino acid sequences are provided. One preferred isolated nucleic acid encodes SEQ ID NO:450:

N-XXIFVXXLXG KXXXLXXXXX XTIEXXKXXI XXXXGIPXXX
XXLXFXGXXL XXGXXLXXYX XXXXXXXXXX LRXX-C

wherein X₁ is selected from M, H, Y, W, Q, T, F, S, R, I, and N; X₂ is selected from Q, L, I, and M; X₆ is selected from K and R; X₇ is selected from T, M, I, C, L, and V; X₉ is selected from T, I, S, E and V; X₁₂ is selected from T, M, and Y; X₁₃ is selected from I, F, H and P; X₁₄ is selected from T, E, D, H, and N; X₁₆ is selected from E, M, T, N, Y, D, and H; X₁₇ is selected from V and C; X₁₈ is selected from E, M, Y, L, H, F, W, S, Q, T, C, N, R, and D; X₁₉ is selected from P and K; X₂₀ is selected from S, D, N, C, A, and W; X₂₁ is selected from D and E; X₂₅ is selected from N, V, I, E, G, M, Q, D, A, L, R, S, K, T, C, and F; X₂₆ is selected from I, V, and L; X₂₈ is selected from A, E, Q, W, I, M, and D; X₂₉ is selected from K, M, L, R, Q, and H; X₃₁ is selected from Q, C, F, W, H, Y, L, R, and M; X₃₂ is selected from D, A, E, and R; X₃₃ is selected from K, H, A, Q, S, V, L, E, M, T, I, F, C, Y, R, N, and W; X₃₄ is selected from E and T; X₃₈ is selected from P, L, C, F, I, V, Y, T, M, H, S, Q, A, W, N, and K; X₃₉ is selected from D, W, E, G, S, L, and Q; X₄₀ is selected from Q, E, and D; X₄₁ is selected from Q, Y, I, C, and V; X₄₂ is selected from R, W, F, H, Y, N, C, and S; X₄₄ is selected from I, A and T; X₄₆ is selected from A, Q, and G; X₄₈ is selected from K, T, M, I, Q, V, R, L, and N; X₄₉ is selected from Q, S, L, M, P, E V, A, D, I, C, G, and N; X₅₁ is selected from E and D; X₅₂ is selected from D and E; X₅₄ is selected from R, Y, M, T, H, F, N, Q, K, and C; X₅₅ is selected from T and R; X₅₇ is selected from S, G, D, N, H, E, A, Q, M, R, and K; X₅₈ is selected from D and S; X₆₀ is selected from N, E, and Q; X₆₁ is selected from I and L; X₆₂ is selected from Q, L, T, V, C, A, M, I and S; X₆₃ is selected from K, I, M, F, and V; X₆₄ is selected from E, D, and S; X₆₅ is selected from S, P, E, K, H, R, A, D, N, and Q; X₆₆ is selected from T, K, R, and E; X₆₇ is selected from L, H, K, R, S, M, C, Y, and T; X₆₈ is selected from H, M, Q, and E; X₆₉ is selected from L, P, R, A, G, C, F, M, and S; X₇₀ is selected from V, L, M, F, and C; X₇₃ is selected from L and M; and X₇₄ is selected from R, Q, V, L, M, C, I, T, E, and K, and combinations thereof, provided that polypeptides encoding SEQ ID NOS:1-3 are excluded (i.e., SEQ ID NOS: 666, 667 and 883).

Another preferred isolated nucleic acid encodes SEQ ID NO:1100:

N-MHHHHHHGGSG XXIFVXXLXG KXXXLXXXXX XTIEXXKXXI
XXXXGIPXXX XXLXFXGXXL XXGXXLXXYX XXXXXXXXXX
LRXX-C

wherein X₁₂ is selected from M, H, Y, W, Q, T, F, S, R, I, and N; X₁₃ is selected from Q, L, I, and M; X₁₇ is selected from K and R; X₁₈ is selected from T, M, I, C, L, and V; X₂₀ is selected from T, I, S, E and V; X₂₃ is selected from T, M, and Y; X₂₄ is selected from I, F, H and P; X₂₅ is selected from T, E, D, H, and N; X₂₇ is selected from E, M, T, N, Y, D, and H; X₂₈ is selected from V and C; X₂₉ is selected from E, M, Y, L, H, F, W, S, Q, T, C, N, R, and D; X₃₀ is selected from P and K; X₃₁ is selected from S, D, N, C, A, and W; X₃₂ is selected from D and E; X₃₆ is selected from N, V, I, E, G, M, Q, D, A, L, R, S, K, T, C, and F; X₃₇ is selected from I, V, and L; X₃₉ is selected from A, E, Q, W, I, M, and D; X₄₀ is selected from K, M, L, R, Q, and H; X₄₂ is selected from Q, C, F, W, H, Y, L, R, and M; X₄₃ is selected from D, A, E, and R; X₄₄ is selected from K, H, A, Q, S, V, L, E, M, T, I, F, C, Y, R, N, and W; X₄₅ is selected from E and T; X₄₉ is selected from P, L, C, F, I, V, Y, T, M, H, S, Q, A, W, N, and K; X₅₀ is selected from D, W, E, G, S, L, and Q; X₅₁ is selected from Q, E, and D; X₅₂ is selected from Q, Y, I, C, and V; X₅₃ is selected from R, W, F, H, Y, N, C, and S; X₅₅ is selected from I, A and T; X₅₇ is selected from A, Q, and G; X₅₉ is selected from K, T, M, I, Q, V, R, L, and N; X₆₀ is selected from Q, S, L, M, P, E V, A, D, I, C, G, and N; X₆₂ is selected from E and D; X₆₃ is selected from D and E; X₆₅ is selected from R, Y, M, T, H, F, N, Q, K, and C; X₆₆ is selected from T and R; X₆₈ is selected from S, G, D, N, H, E, A, Q, M, R, and K; X₆₉ is selected from D and S; X₇₁ is selected from N, E, and Q; X₇₂ is selected from I and L; X₇₃ is selected from Q, L, T, V, C, A, M, I and S; X₇₄ is selected from K, I, M, F, and V; X₇₅ is selected from E, D, and S; X₇₆ is selected from S, P, E, K, H, R, A, D, N, and Q; X₇₇ is selected from T, K, R, and E; X₇₈ is selected from L, H, K, R, S, M, C, Y, and T; X₇₉ is selected from H, M, Q, and E; X₈₀ is selected from L, P, R, A, G, C, F, M, and S; X₈₁ is selected from V, L, M, F, and C; X₈₄ is selected from L and M; and X₈₅ is selected from R, Q, V, L, M, C, I, T, E, and K, and combinations thereof, provided that SEQ ID NO: 3 is excluded.

Preferred isolated polynucleotides (e.g., DNA and their corresponding RNA counterparts) include those that encode Ubvs having an amino acid sequence identity in the range of at least 70% to 100% identity of SEQ ID NOS: 450 and 1100, respectively. Even more preferably, isolated polynucleotides include those that encode Ubvs having an amino acid sequence identity in the range of at least 80% to 100% identity of SEQ ID NOS: 450 and 1100, respectively. Even more preferably, preferred isolated polynucleotides include those that encode Ubvs having an amino acid sequence identity in the range of at least 90% to 100% identity of SEQ ID NOS: 450 and 1100, respectively. Even more preferably, preferred isolated polynucleotides include those that encode Ubvs having an amino acid sequence identity in the range of at least 95% to 100% identity of SEQ ID NOS: 450 and 1100, respectively.

### Preferred Isolated Ubv Polynucleotides Include Those Having Significant Amino Acid Sequence Identity to Reference Sequences.

An isolated polynucleotide that encodes an isolated polypeptide with enhanced HDR activity through interactions with 53BP1 in a manner to influence repair mechanisms at DSB sites is provided. The encoded isolated polypeptide comprises a Ubv having at least 40% amino acid sequence identity to amino acid positions 1-74 of SEQ ID NOS:1, 2, 482, 633, or 450, provided that SEQ ID NOS:1 and 2 are excluded, and those having at least 40% amino acid sequence identity with amino acid positions 12-85 of SEQ ID NOS: 3, 241, 417, or 1100, provided that SEQ ID NO:3 is excluded. Such an isolated polypeptide identity provides enhanced HDR activity through interactions with 53BP1 in a manner to influence repair mechanisms at DSB sites relative to SEQ ID NO:1 under identical conditions.

Preferred isolated polynucleotides encoding such isolated polypeptides include polypeptides those having amino acid sequence identity in the range of at least 50% to 100% identity with amino acid positions 1-74 of SEQ ID NOS:1, 2, 482, 633, or 450, provided that SEQ ID NOS:1 and 2 are excluded, and those having amino acid sequence identity in the range of at least 50% to 100% identity with amino acid positions 12-85 of SEQ ID NOS: 3, 241, 417, or 1100, provided that SEQ ID NO:3 is excluded. Even more preferably, isolated polynucleotides encoding such isolated polypeptides include those having amino acid sequence identity in the range of at least 60% to 100% identity with amino acid positions 1-74 of SEQ ID NOS:1, 2, 482, 633, or 450, provided that SEQ ID NOS:1 and 2 are excluded, and those having amino acid sequence identity in the range of at least 60% to 100% identity with amino acid positions 12-85 of SEQ ID NOS: 3, 241, 417, or 1100, provided that SEQ ID NO:3 is excluded. Even more preferably, isolated polynucleotides encoding such isolated polypeptides include those having amino acid sequence identity in the range of at least 70% to 100% identity with amino acid positions 1-74 of SEQ ID NOS:1, 2, 482, 633, or 450, provided that SEQ ID NOS:1 and 2 are excluded, and those having amino acid sequence identity in the range of at least 70% to 100% identity with amino acid positions 12-85 of SEQ ID NOS: 3, 241, 417, or 1100, provided that SEQ ID NO:3 is excluded. Even more preferably, isolated polynucleotides encoding such isolated polypeptides include those having amino acid sequence identity in the range of at least 80% to 100% identity with amino acid positions 1-74 of SEQ ID NOS:1, 2, 482, 633, or 450, provided that SEQ ID NOS:1 and 2 are excluded, and those having amino acid sequence identity in the range of at least 80% to 100% identity with amino acid positions 12-85 of SEQ ID NOS: 3, 241, 417, or 1100, provided that SEQ ID NO:3 is excluded. Even more preferably, preferred isolated polynucleotides encoding such isolated polypeptides include those having amino acid sequence identity in the range of at least 90% to 100% identity with amino acid positions 1-74 of SEQ ID NOS:1, 2, 482, 633, or 450, provided that SEQ ID NOS:1 and 2 are excluded, and those having amino acid sequence identity in the range of at least 90% to 100% identity with amino acid positions 12-85 of SEQ ID NOS: 3, 241, 417, or 1100, provided that SEQ ID NO:3 is excluded. Even more preferably, preferred isolated polynucleotides encoding such isolated polypeptides include those having amino acid sequence identity in the range of at least 95% to 100% identity with amino acid positions 1-74 of SEQ ID NOS:1, 2, 482, 633, or 450, provided that SEQ ID NOS:1 and 2 are excluded, and those having amino acid sequence identity in the range of at least 95% to 100% identity with amino acid positions 12-85 of SEQ ID NOS: 3, 241, 417, or 1100, provided that SEQ ID NO:3 is excluded.

A preferred isolated polynucleotide encoding such isolated polypeptides within the stated ranges of % amino acid sequence identity to the aforementioned reference polypeptide sequence(s) in the aforementioned ranges, further provide a functional benefit of enhanced HDR rates when compared to HDR rates of an isolated polynucleotide encoding SEQ ID NO:1 under identical conditions. Such enhanced HDR rates can be readily assessed by one of skill in the art based upon the teachings disclosed herein, including evaluations as described previously herein.

Applications

It will be generally understood that the disclosed amino acid substitutions within the ubiquitin polypeptide variants that result in improved affinity for 53BP1 can be generated in the context of the wild-type ubiquitin polypeptide (SEQ ID NO:1) or the i53 ubiquitin polypeptide (SEQ ID NO:2), including tag-free polypeptides and fusion polypeptides having an affinity tag included as part of the ubiquitin polypeptide variants. For example, one skilled in the art will appreciate that untagged versions or differently tagged versions fall within the scope of the disclosed ubiquitin polypeptide variants, including those ubiquitin polypeptide variants having a polyhistidine motif (e.g., a His₆ tag). Accordingly, alternative versions of ubiquitin polypeptide variants may be constructed and function either with or without an affinity tag, such as a polyhistidine tag.

In a first aspect, an isolated polypeptide comprising a ubiquitin polypeptide variant is provided. The isolated polypeptide comprises at least one member selected from one of the following groups:

SEQ ID NO:450, wherein X₁ is selected from M, H, Y, W, Q, T, F, S, R, I, and N; X₂ is selected from Q, L, I, and M; X₆ is selected from K and R; X₇ is selected from T, M, I, C, L, and V; X₉ is selected from T, I, S, E and V; X₁₂ is selected from T, M, and Y; X₁₃ is selected from I, F, H and P; X₁₄ is selected from T, E, D, H, and N; X₁₆ is selected from E, M, T, N, Y, D, and H; X₁₇ is selected from V and C; X₁₈ is selected from E, M, Y, L, H, F, W, S, Q, T, C, N, R, and D; X₁₉ is selected from P and K; X₂₀ is selected from S, D, N, C, A, and W; X₂₁ is selected from D and E; X₂₅ is selected from N, V, I, E, G, M, Q, D, A, L, R, S, K, T, C, and F; X₂₆ is selected from I, V, and L; X₂₈ is selected from A, E, Q, W, I, M, and D; X₂₉ is selected from K, M, L, R, Q, and H; X₃1 is selected from Q, C, F, W, H, Y, L, R, and M; X₃₂ is selected from D, A, E, and R; X₃₃ is selected from K, H, A, Q, S, V, L, E, M, T, I, F, C, Y, R, N, and W; X₃₄ is selected from E and T; X₃₈ is selected from P, L, C, F, I, V, Y, T, M, H, S, Q, A, W, N, and K; X₃₉ is selected from D, W, E, G, S, L, and Q; X₄₀ is selected from Q, E, and D; X₄₁ is selected from Q, Y, I, C, and V; X₄₂ is selected from R, W, F, H, Y, N, C, and S; X₄₄ is selected from I, A and T; X₄₆ is selected from A, Q, and G; X₄₈ is selected from K, T, M, I, Q, V, R, L, and N; X₄₉ is selected from Q, S, L, M, P, E V, A, D, I, C, G, and N; X₅₁ is selected from E and D; X₅₂ is selected from D and E; X₅₄ is selected from R, Y, M, T, H, F, N, Q, K, and C; X₅₅ is selected from T and R; X₅₇ is selected from S, G, D, N, H, E, A, Q, M, R, and K; X₅₈ is selected from D and S; X₆₀ is selected from N, E, and Q; X₆₁ is selected from I and L; X₆₂ is selected from Q, L, T, V, C, A, M, I and S; X₆₃ is selected from K, I, M, F, and V; X₆₄ is selected from E, D, and S; X₆₅ is selected from S, P, E, K, H, R, A, D, N, and Q; X₆₆ is selected from T, K, R, and E; X₆₇ is selected from L, H, K, R, S, M, C, Y, and T; X₆₈ is selected from H, M, Q, and E; X₆₉ is selected from L, P, R, A, G, C, F, M, and S; X₇₀ is selected from V, L, M, F, and C; X₇₃ is selected from L and M; and X₇₄ is selected from R, Q, V, L, M, C, I, T, E, and K, and combinations thereof, provided that SEQ ID NOS:1-3 are excluded; and

at least one member selected from the group of SEQ ID NOs:452-665.

In a first respect, the isolated polypeptide comprises a ubiquitin polypeptide variant selected from SEQ ID NO:450, wherein X₁ is selected from M, H, Y, W, Q, T, F, S, R, I, and N; X₂ is selected from Q, L, I, and M; X₆ is selected from K and R; X₇ is selected from T, M, I, C, L, and V; X₉ is selected from T, I, S, E and V; X₁₂ is selected from T, M, and Y; X₁₃ is selected from I, F, H and P; X₁₄ is selected from T, E, D, H, and N; X₁₆ is selected from E, M, T, N, Y, D, and H; X₁₇ is selected from V and C; X₁₈ is selected from E, M, Y, L, H, F, W, S, Q, T, C, N, R, and D; X₁₉ is selected from P and K; X₂₀ is selected from S, D, N, C, A, and W; X₂₁ is selected from D and E; X₂₅ is selected from N, V, I, E, G, M, Q, D, A, L, R, S, K, T, C, and F; X₂₆ is selected from I, V, and L; X₂₈ is selected from A, E, Q, W, I, M, and D; X₂₉ is selected from K, M, L, R, Q, and H; X₃₁ is selected from Q, C, F, W, H, Y, L, R, and M; X₃₂ is selected from D, A, E, and R; X₃₃ is selected from K, H, A, Q, S, V, L, E, M, T, I, F, C, Y, R, N, and W; X₃₄ is selected from E and T; X₃₈ is selected from P, L, C, F, I, V, Y, T, M, H, S, Q, A, W, N, and K; X₃₉ is selected from D, W, E, G, S, L, and Q; X₄₀ is selected from Q, E, and D; X₄₁ is selected from Q, Y, I, C, and V; X₄₂ is selected from R, W, F, H, Y, N, C, and S; X₄₄ is selected from I, A and T; X₄₆ is selected from A, Q, and G; X₄₈ is selected from K, T, M, I, Q, V, R, L, and N; X₄₉ is selected from Q, S, L, M, P, E V, A, D, I, C, G, and N; X₅₁ is selected from E and D; X₅₂ is selected from D and E; X₅₄ is selected from R, Y, M, T, H, F, N, Q, K, and C; X₅₅ is selected from T and R; X₅₇ is selected from S, G, D, N, H, E, A, Q, M, R, and K; X₅₈ is selected from D and S; X₆₀ is selected from N, E, and Q; X₆₁ is selected from I and L; X₆₂ is selected from Q, L, T, V, C, A, M, I and S; X₆₃ is selected from K, I, M, F, and V; X₆₄ is selected from E, D, and S; X₆₅ is selected from S, P, E, K, H, R, A, D, N, and Q; X₆₆ is selected from T, K, R, and E; X₆₇ is selected from L, H, K, R, S, M, C, Y, and T; X₆₈ is selected from H, M, Q, and E; X₆₉ is selected from L, P, R, A, G, C, F, M, and S; X₇₀ is selected from V, L, M, F, and C; X₇₃ is selected from L and M; and X₇₄ is selected from R, Q, V, L, M, C, I, T, E, and K, and combinations thereof, provided that SEQ ID NOS:1-3 are excluded. In a second respect, the isolated polypeptide shares amino acid sequence identity in the range of at least 40% to 100% identity of SEQ ID NO:1. In a third respect, the isolated polypeptide shares amino acid sequence identity in the range of at least 50% to 100% identity of SEQ ID NO:1. In a fourth respect, the isolated polypeptide shares amino acid sequence identity in the range of at least 60% to 100% identity of SEQ ID NO:1. In a fifth respect, the isolated polypeptide shares amino acid sequence identity in the range of at least 70% to 100% identity of SEQ ID NO:1. In a sixth respect, the isolated polypeptide shares amino acid sequence identity in the range of at least 80% to 100% identity of SEQ ID NO:1. In a seventh respect, the isolated polypeptide shares amino acid sequence identity in the range of at least 90% to 100% identity of SEQ ID NO:1. In an eighth respect, the isolated polypeptide shares amino acid sequence identity in the range of at least 95% to 100% identity of SEQ ID NO:1.

In a second aspect, an isolated polypeptide comprising an isolated fusion polypeptide having an Ubv amino acid sequence with an N-terminal His₆-tag is provided. The isolated fusion polypeptide comprises at least one member selected from the following: an isolated fusion polypeptide comprising SEQ ID NO: 1100, wherein X₁₂ is selected from M, H, Y, W, Q, T, F, S, R, I, and N; X₁₃ is selected from Q, L, I, and M; X₁₇ is selected from K and R; X₁₈ is selected from T, M, I, C, L, and V; X₂₀ is selected from T, I, S, E and V; X₂₃ is selected from T, M, and Y; X₂₄ is selected from I, F, H and P; X₂₅ is selected from T, E, D, H, and N; X₂₇ is selected from E, M, T, N, Y, D, and H; X₂₈ is selected from V and C; X₂₉ is selected from E, M, Y, L, H, F, W, S, Q, T, C, N, R, and D; X₃₀ is selected from P and K; X₃₁ is selected from S, D, N, C, A, and W; X₃₂ is selected from D and E; X₃₆ is selected from N, V, I, E, G, M, Q, D, A, L, R, S, K, T, C, and F; X₃₇ is selected from I, V, and L; X₃₉ is selected from A, E, Q, W, I, M, and D; X₄₀ is selected from K, M, L, R, Q, and H; X₄₂ is selected from Q, C, F, W, H, Y, L, R, and M; X₄₃ is selected from D, A, E, and R; X₄₄ is selected from K, H, A, Q, S, V, L, E, M, T, I, F, C, Y, R, N, and W; X₄₅ is selected from E and T; X₄₉ is selected from P, L, C, F, I, V, Y, T, M, H, S, Q, A, W, N, and K; X₅₀ is selected from D, W, E, G, S, L, and Q; X₅₁ is selected from Q, E, and D; X₅₂ is selected from Q, Y, I, C, and V; X₅₃ is selected from R, W, F, H, Y, N, C, and S; X₅₅ is selected from I, A and T; X₅₇ is selected from A, Q, and G; X₅₉ is selected from K, T, M, I, Q, V, R, L, and N; X₆₀ is selected from Q, S, L, M, P, E V, A, D, I, C, G, and N; X₆₂ is selected from E and D; X₆₃ is selected from D and E; X₆₅ is selected from R, Y, M, T, H, F, N, Q, K, and C; X₆₆ is selected from T and R; X₆₈ is selected from S, G, D, N, H, E, A, Q, M, R, and K; X₆₉ is selected from D and S; X₇₁ is selected from N, E, and Q; X₇₂ is selected from I and L; X₇₃ is selected from Q, L, T, V, C, A, M, I and S; X₇₄ is selected from K, I, M, F, and V; X₇₅ is selected from E, D, and S; X₇₆ is selected from S, P, E, K, H, R, A, D, N, and Q; X₇₇ is selected from T, K, R, and E; X₇₈ is selected from L, H, K, R, S, M, C, Y, and T; X₇₉ is selected from H, M, Q, and E; X₈₀ is selected from L, P, R, A, G, C, F, M, and S; X₈₁ is selected from V, L, M, F, and C; X₈₄ is selected from L and M; and X₈₅ is selected from R, Q, V, L, M, C, I, T, E, and K, and combinations thereof, provided that SEQ ID NO: 3 is excluded; and an isolated fusion polypeptide comprising at least one member selected SEQ ID NOS:235-244 and 246-449.

In a first respect, an isolated polypeptide comprising an isolated fusion polypeptide having an Ubv amino acid sequence with an N-terminal His₆-tag is provided. The isolated fusion polypeptide comprises at least one member selected from the following: an isolated fusion polypeptide comprising SEQ ID NO: 1100, wherein X₁₂ is selected from M, H, Y, W, Q, T, F, S, R, I, and N; X₁₃ is selected from Q, L, I, and M; X₁₇ is selected from K and R; X₁₈ is selected from T, M, I, C, L, and V; X₂₀ is selected from T, I, S, E and V; X₂₃ is selected from T, M, and Y; X₂₄ is selected from I, F, H and P; X₂₅ is selected from T, E, D, H, and N; X₂₇ is selected from E, M, T, N, Y, D, and H; X₂₈ is selected from V and C; X₂₉ is selected from E, M, Y, L, H, F, W, S, Q, T, C, N, R, and D; X₃₀ is selected from P and K; X₃₁ is selected from S, D, N, C, A, and W; X₃₂ is selected from D and E; X₃₆ is selected from N, V, I, E, G, M, Q, D, A, L, R, S, K, T, C, and F; X₃₇ is selected from I, V, and L; X₃₉ is selected from A, E, Q, W, I, M, and D; X₄₀ is selected from K, M, L, R, Q, and H; X₄₂ is selected from Q, C, F, W, H, Y, L, R, and M; X₄₃ is selected from D, A, E, and R; X₄₄ is selected from K, H, A, Q, S, V, L, E, M, T, I, F, C, Y, R, N, and W; X₄₅ is selected from E and T; X₄₉ is selected from P, L, C, F, I, V, Y, T, M, H, S, Q, A, W, N, and K; X₅₀ is selected from D, W, E, G, S, L, and Q; X₅₁ is selected from Q, E, and D; X₅₂ is selected from Q, Y, I, C, and V; X₅₃ is selected from R, W, F, H, Y, N, C, and S; X₅₅ is selected from I, A and T; X₅₇ is selected from A, Q, and G; X₅₉ is selected from K, T, M, I, Q, V, R, L, and N; X₆₀ is selected from Q, S, L, M, P, E V, A, D, I, C, G, and N; X₆₂ is selected from E and D; X₆₃ is selected from D and E; X₆₅ is selected from R, Y, M, T, H, F, N, Q, K, and C; X₆₆ is selected from T and R; X₆₈ is selected from S, G, D, N, H, E, A, Q, M, R, and K; X₆₉ is selected from D and S; X₇₁ is selected from N, E, and Q; X₇₂ is selected from I and L; X₇₃ is selected from Q, L, T, V, C, A, M, I and S; X₇₄ is selected from K, I, M, F, and V; X₇₅ is selected from E, D, and S; X₇₆ is selected from S, P, E, K, H, R, A, D, N, and Q; X₇₇ is selected from T, K, R, and E; X₇₈ is selected from L, H, K, R, S, M, C, Y, and T; X₇₉ is selected from H, M, Q, and E; X₈₀ is selected from L, P, R, A, G, C, F, M, and S; X₈₁ is selected from V, L, M, F, and C; X₈₄ is selected from L and M; and X₈₅ is selected from R, Q, V, L, M, C, I, T, E, and K, and combinations thereof, provided that SEQ ID NO: 3 is excluded. In a second respect, the isolated polypeptide of SEQ ID 1100 encompassing amino acid positions 12-85 shares amino acid sequence identity in the range of at least 40% to 100% identity of SEQ ID NO:1. In a third respect, the isolated polypeptide of SEQ ID 1100 encompassing amino acid positions 12-85 shares amino acid sequence identity in the range of at least 50% to 100% identity of SEQ ID NO:1. In a fourth respect, the isolated polypeptide of SEQ ID 1100 encompassing amino acid positions 12-85 shares amino acid sequence identity in the range of at least 60% to 100% identity of SEQ ID NO:1. In a fifth respect, the isolated polypeptide of SEQ ID 1100 encompassing amino acid positions 12-85 shares amino acid sequence identity in the range of at least 70% to 100% identity of SEQ ID NO:1. In a sixth respect, the isolated polypeptide of SEQ ID 1100 encompassing amino acid positions 12-85 shares amino acid sequence identity in the range of at least 80% to 100% identity of SEQ ID NO:1. In a seventh respect, the isolated polypeptide of SEQ ID 1100 encompassing amino acid positions 12-85 shares amino acid sequence identity in the range of at least 90% to 100% identity of SEQ ID NO:1. In an eighth respect, the isolated polypeptide of SEQ ID 1100 encompassing amino acid positions 12-85 shares amino acid sequence identity in the range of at least 95% to 100% identity of SEQ ID NO:1.

In a third aspect, an isolated polypeptide that enhances rates of HDR through interactions with 53BP1 in a manner to influence repair mechanisms at DSB sites is provided. The isolated polypeptide includes a Ubv having at least 40% amino acid sequence identity to amino acid positions 1-74 of SEQ ID NOS:1, 2, 482, 633, or 450, provided that SEQ ID NOS:1 and 2 are excluded, and those having at least 40% amino acid sequence identity with amino acid positions 12-85 of SEQ ID NOS: 3, 241, 417, or 1100, provided that SEQ ID NO:3 is excluded. The isolated polypeptide provides enhanced HDR activity through interactions with 53BP1 in a manner to influence repair mechanisms at DSB sites relative to SEQ ID NO:1 under identical conditions.

In a first respect, the isolated polypeptide includes a Ubv having at least 50% amino acid sequence identity to amino acid positions 1-74 of SEQ ID NOS:1, 2, 482, 633, or 450, provided that SEQ ID NOS:1 and 2 are excluded, and those having at least 50% amino acid sequence identity with amino acid positions 12-85 of SEQ ID NOS: 3, 241, 417, or 1100, provided that SEQ ID NO:3 is excluded. In a second respect, the isolated polypeptide includes a Ubv having at least 60% amino acid sequence identity to amino acid positions 1-74 of SEQ ID NOS:1, 2, 482, 633, or 450, provided that SEQ ID NOS:1 and 2 are excluded, and those having at least 60% amino acid sequence identity with amino acid positions 12-85 of SEQ ID NOS: 3, 241, 417, or 1100, provided that SEQ ID NO:3 is excluded. In a third respect, the isolated polypeptide includes a Ubv having at least 70% amino acid sequence identity to amino acid positions 1-74 of SEQ ID NOS:1, 2, 482, 633, or 450, provided that SEQ ID NOS:1 and 2 are excluded, and those having at least 70% amino acid sequence identity with amino acid positions 12-85 of SEQ ID NOS: 3, 241, 417, or 1100, provided that SEQ ID NO:3 is excluded. In a fourth respect, the isolated polypeptide includes a Ubv having at least 80% amino acid sequence identity to amino acid positions 1-74 of SEQ ID NOS:1, 2, 482, 633, or 450, provided that SEQ ID NOS:1 and 2 are excluded, and those having at least 80% amino acid sequence identity with amino acid positions 12-85 of SEQ ID NOS: 3, 241, 417, or 1100, provided that SEQ ID NO:3 is excluded. In a fifth respect, the isolated polypeptide includes a Ubv having at least 90% amino acid sequence identity to amino acid positions 1-74 of SEQ ID NOS:1, 2, 482, 633, or 450, provided that SEQ ID NOS:1 and 2 are excluded, and those having at least 90% amino acid sequence identity with amino acid positions 12-85 of SEQ ID NOS: 3, 241, 417, or 1100, provided that SEQ ID NO:3 is excluded. In a sixth respect, the isolated polypeptide includes a Ubv having at least 95% amino acid sequence identity to amino acid positions 1-74 of SEQ ID NOS:1, 2, 482, 633, or 450, provided that SEQ ID NOS:1 and 2 are excluded, and those having at least 95% amino acid sequence identity with amino acid positions 12-85 of SEQ ID NOS: 3, 241, 417, or 1100, provided that SEQ ID NO:3 is excluded.

In a fourth aspect, an isolated polynucleotide is provided. The isolated polynucleotide encodes the isolated polypeptide of any of the first, second, or third aspects.

In a fifth aspect, an isolated polynucleotide encoding a ubiquitin polypeptide variant is provided. The isolated polynucleotide comprises at least one member selected from SEQ ID NOS:669-682, 885-890, and 892-1099, and the corresponding RNA counterparts thereof.

In a sixth aspect, a vector comprising an isolated polynucleotide encoding a ubiquitin polypeptide variant is provided. The isolated polynucleotide comprises at least one member selected from SEQ ID NOS:669-682, 885-890, and 892-1099, and the corresponding RNA counterparts thereof.

In a seventh aspect, a cell or cell line comprising the isolated polypeptide of the first, second, or third aspects, the isolated polynucleotide of the fourth or fifth aspects, or the vector of the sixth aspect.

In an eighth aspect, a method of suppressing 53BP1 recruitment to DNA double-strand break sites in a cell is provided. The method includes a step of administering to the cell the isolated polypeptide of the first, second, or third aspects, the isolated polynucleotide of the fourth or fifth aspects, or the vector of the sixth aspect.

In a ninth aspect, a method of increasing homologous recombination in a cell is provided. The method includes a step of administering to the cell the isolated polypeptide of the first, second, or third aspects, the isolated polynucleotide of the fourth or fifth aspects, or the vector of the sixth aspect.

In a tenth aspect, a method of editing a gene in a cell using a CRISPR system is provided. The method includes a step of administering to the cell the isolated polypeptide of the first, second, or third aspects, the isolated polynucleotide of the fourth or fifth aspects, or the vector of the sixth aspect.

In an eleventh aspect, a method of gene targeting in a cell is provided. The method includes a step of administering to the cell the isolated polypeptide of the first, second, or third aspects, the isolated polynucleotide of the fourth or fifth aspects, or the vector of the sixth aspect.

In a twelfth aspect, a composition comprising the isolated polypeptide the isolated polypeptide of the first, second or third aspects is provided.

In an thirteenth aspect, a kit comprising the isolated polypeptide of the first, second, or third aspects, the isolated polynucleotide of the fourth or fifth aspects, or the vector of the sixth aspect. In a first respect, the kit additionally includes one or more components of a gene editing system. In this regard, the gene editing system is a CRISPR system.

In a fourteenth aspect, a method of performing a medically therapeutic procedure is provided. The includes the step of performing genome editing according to any of the tenth or eleventh aspects.

In a fifteenth aspect, a method of screening for amino acid changes in a first polypeptide that improve affinity of the first polypeptide for a second polypeptide is provided. The method includes a step of using the BACTH system with a reporter gene under control of cAMP regulated promoter to allow fluorescence activated cell sorting based on protein-protein interaction affinity between the first polypeptide and the second polypeptide to screen for improved affinity variants of the first polypeptide.

The polypeptides and polynucleotides disclosed herein may be used in a broad spectrum of applications. The polypeptides and polynucleotides disclosed herein may be used for the detection and quantitative determination as well as for the separation and isolation of 53BP1. The polypeptides and polynucleotides disclosed herein may be used in genomic engineering, epigenomic engineering, genome targeting, and genome editing. The polypeptides and polynucleotides disclosed herein may be used to modify repair pathways, activate or stimulate HDR or homology-based genome editing, inhibit 53BP1 recruitment to DSB sites or damaged chromatin in a cell or modulate DNA end resection. In an aspect, the polypeptides and polynucleotides disclosed herein are used in combination with a gene editing system. The disclosure also provides the use of the polypeptides and polynucleotides disclosed herein as medicaments.

### EXAMPLES

### Example 1. A Two-Hybrid Screen Identified a Variety of Mutations that May Increase Ubiquitin Variant Affinity for 53BP1

In order to identify mutations that improve the affinity of i53 for 53BP1, the bacterial adenylate cyclase two-hybrid system (BACTH system) was used to screen for interaction between the two proteins. This method makes use of a B. pertussis calmodulin-dependent adenylate cyclase toxin. The catalytic domain of the toxin can be separated into two fragments (T18 and T25) that are able to associate in the presence of calmodulin but have minimal activity in its absence [21, 22]. If bait and prey proteins fused to T18 and T25 interact, then the catalytic activity is restored and cAMP is produced. In E. coli, cAMP binds to catabolite activator protein (CAP) that acts as a transcriptional activator for several genes. By expressing these fusion proteins in an E. coli strain that lacks endogenous adenylate cyclase and naturally lacks calmodulin, cAMP regulated protein expression can be used as a readout of bait-prey interaction [23]. We engineered the screen so that eGFP will be expressed under the control of a cAMP-regulated promoter. The coding sequence for a fragment of 53BP1 (a.a. 1221-1718) containing the i53 interacting regions and i53 were cloned into T18 and T25 adenylate cyclase expression plasmids such that fusion proteins of each would be expressed. If a Ubv interacts with 53BP1, the T18 and T25 fragments will be brought together, adenylate cyclase activity will be restored, cAMP will be produced, and some portion of the bacterial population will be GFP positive.

A plasmid library was made consisting of Ubv-adenylate cyclase fragment fusion protein plasmids that had on average a single codon within the i53 coding region exchanged for a random NNK codon. Plasmids were transformed into DHM1 cells that lack endogenous adenylate cyclase and contain the plasmid for expression of the 53BP1 fragment fused to one of the adenylate cyclase fragments. Expression of eGFP was used as a readout of bait-prey interaction using fluorescence activated cell sorting (FACS) to sort for GFP positive bacteria. Plasmid DNA was isolated from both the sorted GFP positive bacteria (Positive) and from the original pre-sort population (Input) and was sequenced using NGS. Counts were merged for mutations that result in the same amino acid change using Enrich2 [25]. Enrichment was calculated as enrichment=log 2((read count for an amino acid change in the positive population/read count for an amino acid change in the input)/(synonymous change read count in the positive population/synonymous change read count in the input)). A positive enrichment value indicates that mutations resulting in a particular amino acid substitution result in a higher percent of GFP positive bacteria than synonymous mutations and therefore indicates that the amino acid change may improve i53 affinity for 53BP1. For each experiment, DHM1 cells were transformed with the Ubv fusion protein plasmid library in two separate replicates using a gene pulser (Bio-Rad). The i53-adenylate cyclase fragment fusion protein (published i53 peptide, SEQ ID NO:2) plasmid was also introduced separately as a control to estimate selection pressure. Cells were then grown and sorted using FACS and GFP positive cells were collected. Two separate experiments were conducted on separate days using different levels of selection pressure resulting in a different percent GFP positive for the i53 population (i.e. for cells that express published i53 peptide (SEQ ID NO:2) fused to one of the adenylate cyclase fragments). Experiment one had an i53 percent positive of approximately 30 and experiment two had an i53 percent GFP positive of approximately 1700.

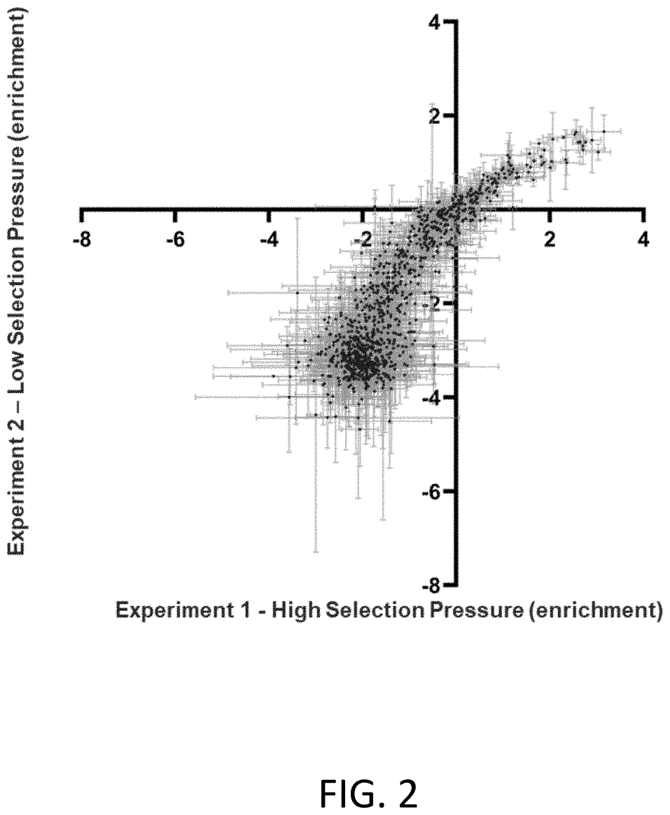

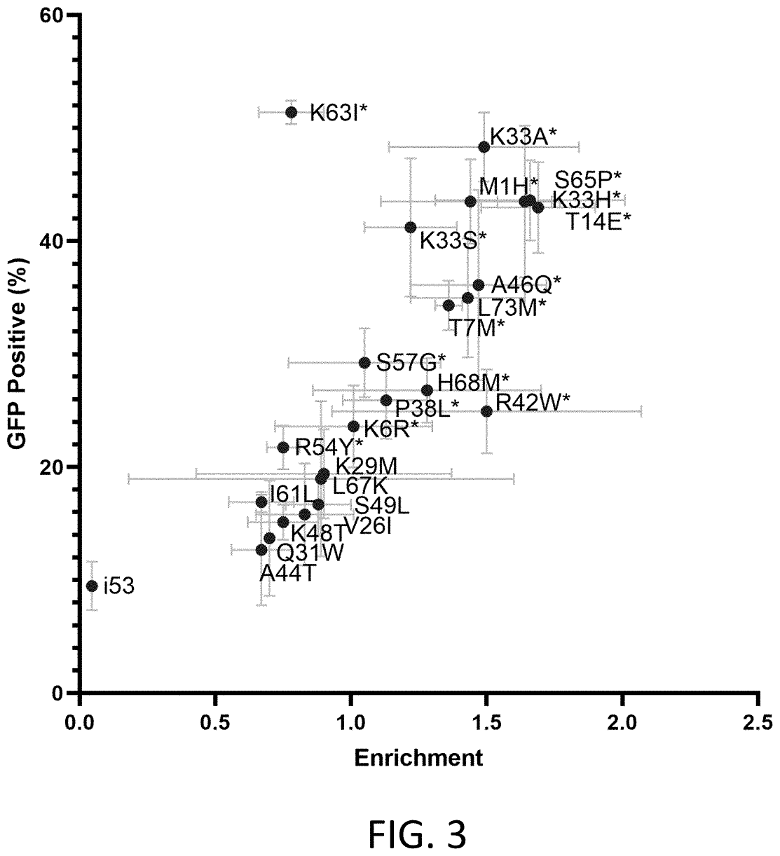

There was a high degree of correlation between the two experiments and between replicates (FIG. 2). From these screens, about 230 amino acid changes were identified for which the average enrichment was positive for at least one of the experiments (Table 1). These amino acid changes resulted in increased reporter gene (GFP) expression in our two-hybrid system and potentially improve the affinity of i53 for 53BP 1. To validate that the amino acid changes identified from the pooled screen are reproducible on an individual basis, 24 amino acid changes identified from this screen were introduced individually into the i53 fusion protein plasmid and tested by flow cytometry for their effect on the percent positive population relative to i53 (FIG. 3). There was a strong correlation between the enrichment measured from the pooled screen and the percent reporter positive cells when mutations were screened individually. Of the 24 mutations tested individually, 16/24 mutations had a statistically significant increase in percent positive relative to i53 wild type (Table 2).

TABLE 1
Amino acid changes with positive average enrichment in at least one experiment¹

| | Amino | Experiment 1 - high selection pressure | | | Experiment 2-low selection pressure | | |
|---|---|---|---|---|---|---|---|
| SEQ ID | acid | Rep 1 | Rep 2 | Average | Rep 1 | Rep 2 | Average |
| NO: | change | Enrichment | Enrichment | Enrichment | Enrichment | Enrichment | Enrichment |
| 4 | M1H | 2.64 | 2.66 | 2.65 | 1.68 | 1.21 | 1.44 |
| 5 | M1Y | 2.56 | 2.16 | 2.36 | 1.18 | 0.81 | 1.00 |
| 6 | M1W | 1.95 | 1.71 | 1.83 | 0.99 | 0.92 | 0.96 |
| 7 | M1Q | 1.23 | 1.38 | 1.31 | 0.58 | 0.79 | 0.68 |
| 8 | M1T | 0.79 | 0.56 | 0.68 | 0.63 | 0.60 | 0.61 |
| 9 | M1F | 0.71 | 0.90 | 0.80 | 0.39 | 0.33 | 0.36 |
| 10 | M1S | 0.55 | 0.21 | 0.38 | 0.27 | 0.41 | 0.34 |
| 11 | M1I | 0.11 | 0.01 | 0.06 | −0.18 | 0.19 | 0.00 |
| 12 | M1R | −0.01 | 0.22 | 0.10 | 0.11 | 0.30 | 0.21 |
| 13 | M1N | −0.37 | −0.13 | −0.25 | −0.04 | 0.08 | 0.02 |
| 14 | K6R | 1.84 | 1.91 | 1.87 | 1.22 | 0.80 | 1.01 |
| 15 | T7M | 2.72 | 2.68 | 2.70 | 1.39 | 1.32 | 1.36 |
| 16 | T7I | 1.70 | 1.63 | 1.66 | 1.04 | 1.02 | 1.03 |
| 17 | T7C | 1.16 | 1.23 | 1.20 | 0.90 | 0.69 | 0.79 |
| 18 | T7L | 0.62 | 0.61 | 0.61 | −0.07 | −0.31 | −0.19 |
| 19 | T7V | 0.24 | 0.42 | 0.33 | −0.04 | 0.09 | 0.02 |
| 20 | T9S | 0.34 | 0.47 | 0.40 | 0.19 | 0.17 | 0.18 |
| 21 | T9I | 0.30 | 0.61 | 0.46 | −0.01 | 0.52 | 0.25 |
| 22 | T9E | 0.37 | −0.44 | −0.04 | 0.02 | 0.63 | 0.33 |
| 23 | T9V | 0.32 | −0.21 | 0.06 | −0.36 | 0.06 | −0.15 |
| 24 | T12M | 1.09 | 0.86 | 0.97 | 0.65 | 1.03 | 0.84 |
| 25 | T12Y | 0.14 | 0.36 | 0.25 | 0.52 | 0.17 | 0.35 |
| 26 | I13F | 1.04 | 0.03 | 0.53 | −0.26 | 0.07 | −0.09 |
| 27 | I13H | −0.27 | 1.28 | 0.50 | −2.48 | −0.71 | −1.60 |
| 28 | I13P | 2.15 | −0.99 | 0.58 | −3.84 | −4.62 | −4.23 |
| 29 | T14D | 2.90 | 2.86 | 2.88 | 1.65 | 1.40 | 1.53 |
| 30 | T14E | 2.88 | 2.88 | 2.88 | 1.84 | 1.54 | 1.69 |
| 31 | T14N | 2.84 | 2.69 | 2.76 | 1.50 | 1.37 | 1.44 |
| 32 | T14H | 2.63 | 2.50 | 2.56 | 1.47 | 1.84 | 1.65 |
| 33 | E16T | 1.12 | 0.64 | 0.88 | 0.27 | 0.33 | 0.30 |
| 34 | E16M | 0.71 | 0.45 | 0.58 | 0.93 | 0.55 | 0.74 |
| 35 | E16Y | 0.23 | 0.47 | 0.35 | −0.32 | −0.11 | −0.22 |
| 36 | E16H | 0.00 | 0.05 | 0.03 | −0.21 | −0.05 | −0.13 |
| 37 | E16N | −0.32 | 0.54 | 0.11 | 0.34 | 0.18 | 0.26 |
| 38 | E16D | −0.69 | 0.30 | −0.20 | 0.42 | −0.15 | 0.13 |
| 39 | V17C | −2.27 | 0.12 | −1.08 | 0.60 | −0.23 | 0.19 |
| 40 | E18Y | 1.28 | 0.17 | 0.72 | 1.20 | 0.44 | 0.82 |
| 41 | E18M | 1.08 | 0.80 | 0.94 | 0.79 | 0.65 | 0.72 |
| 42 | E18Q | 0.61 | 0.83 | 0.72 | 0.02 | 0.05 | 0.03 |
| 43 | E18H | 0.39 | 1.96 | 1.17 | 0.24 | −0.11 | 0.07 |
| 44 | E18F | 0.09 | 0.93 | 0.51 | 0.22 | 0.87 | 0.54 |
| 45 | E18W | 0.06 | 0.31 | 0.18 | 0.80 | 0.69 | 0.75 |
| 46 | E18L | 0.75 | 0.76 | 0.75 | 0.62 | 0.71 | 0.66 |
| 47 | E18S | 0.47 | 0.49 | 0.48 | −0.01 | 0.67 | 0.33 |
| 48 | E18R | −1.22 | −0.28 | −0.75 | −0.24 | 0.36 | 0.06 |
| 49 | E18T | −0.33 | −0.21 | −0.27 | 0.23 | 0.34 | 0.28 |
| 50 | E18N | 0.34 | −1.23 | −0.45 | −0.64 | 0.85 | 0.10 |
| 51 | E18D | −0.69 | −1.02 | −0.85 | 1.65 | −1.37 | 0.14 |
| 52 | E18C | −0.06 | 0.52 | 0.23 | −0.82 | 0.14 | −0.34 |
| 53 | P19K | 2.12 | −0.12 | 1.00 | −0.26 | −1.10 | −0.68 |
| 54 | S20A | 0.30 | −0.02 | 0.14 | −0.26 | −0.25 | −0.26 |
| 55 | S20N | 0.00 | 0.61 | 0.31 | 0.12 | −0.61 | −0.25 |
| 56 | S20D | −0.14 | −0.72 | −0.43 | 0.83 | 0.18 | 0.50 |
| 57 | S20C | −0.36 | 0.20 | −0.08 | −0.10 | 0.21 | 0.05 |
| 58 | S20W | −0.97 | 0.25 | −0.36 | 0.10 | 0.24 | 0.17 |
| 59 | D21E | 0.20 | 0.82 | 0.51 | −0.63 | 0.16 | −0.23 |
| 60 | N25C | 0.45 | −0.19 | 0.13 | 0.31 | 0.21 | 0.26 |
| 61 | N25G | 1.06 | 0.35 | 0.71 | 0.57 | 0.19 | 0.38 |
| 62 | N25I | 0.85 | 0.81 | 0.83 | 0.41 | 0.39 | 0.40 |
| 63 | N25T | −0.19 | 0.44 | 0.12 | 0.33 | 0.24 | 0.29 |
| 64 | N25V | 0.76 | 1.01 | 0.89 | 0.58 | 0.56 | 0.57 |
| 65 | N25M | 0.50 | 0.50 | 0.50 | 0.46 | 0.43 | 0.45 |
| 66 | N25L | 0.47 | 0.39 | 0.43 | 0.26 | 0.19 | 0.23 |
| 67 | N25F | 0.42 | 0.34 | 0.38 | 0.12 | −0.20 | −0.04 |
| 68 | N25E | 0.28 | 0.84 | 0.56 | 0.61 | 0.64 | 0.62 |
| 69 | N25R | 0.25 | 0.36 | 0.31 | 0.25 | 0.34 | 0.29 |
| 70 | N25Q | 0.24 | 1.21 | 0.72 | 0.15 | 0.22 | 0.18 |
| 71 | N25S | 0.18 | 0.34 | 0.26 | 0.27 | 0.38 | 0.33 |
| 72 | N25A | 0.12 | 0.49 | 0.30 | 0.39 | 0.40 | 0.40 |
| 73 | N25D | 0.11 | 0.54 | 0.33 | 0.49 | 0.41 | 0.45 |
| 74 | N25K | 0.11 | 0.54 | 0.32 | 0.17 | 0.24 | 0.21 |
| 75 | V26I | 0.99 | 1.25 | 1.12 | 0.96 | 0.70 | 0.83 |
| 76 | V26L | 0.40 | 0.69 | 0.55 | 0.52 | 0.58 | 0.55 |
| 77 | A28D | −0.25 | 0.29 | 0.02 | −0.24 | −0.58 | −0.41 |
| 78 | A28I | −0.45 | 0.56 | 0.05 | 0.37 | −0.28 | 0.05 |
| 79 | A28M | 0.49 | −0.42 | 0.03 | −0.51 | 0.12 | −0.19 |
| 80 | A28W | 0.48 | −0.41 | 0.03 | 0.41 | −0.17 | 0.12 |
| 81 | A28Q | 0.47 | 0.42 | 0.44 | 0.59 | −0.31 | 0.14 |
| 82 | A28E | 0.23 | 0.40 | 0.31 | 0.53 | 0.32 | 0.42 |
| 83 | K29M | 1.72 | 1.48 | 1.60 | 1.23 | 0.57 | 0.90 |
| 84 | K29H | 0.02 | 0.32 | 0.17 | −3.26 | −2.20 | −2.73 |
| 85 | K29L | 0.15 | 0.07 | 0.11 | 0.21 | 0.30 | 0.26 |
| 86 | K29R | −0.07 | 0.05 | −0.01 | 0.45 | 0.10 | 0.28 |
| 87 | K29Q | −0.21 | 0.31 | 0.05 | −0.26 | 0.17 | −0.04 |
| 88 | Q31C | 1.49 | 0.88 | 1.19 | 1.49 | 1.66 | 1.57 |
| 89 | Q31W | 0.97 | 1.26 | 1.11 | 0.71 | 0.70 | 0.70 |
| 90 | Q31R | 0.66 | −0.37 | 0.15 | 0.14 | −0.58 | −0.22 |
| 91 | Q31H | 0.66 | −0.29 | 0.19 | 0.13 | 0.09 | 0.11 |
| 92 | Q31M | −0.84 | −2.63 | −1.74 | −0.05 | 0.18 | 0.07 |
| 93 | Q31F | 0.95 | 1.39 | 1.17 | 1.04 | 1.14 | 1.09 |
| 94 | Q31L | 0.71 | 0.11 | 0.41 | −0.23 | −0.06 | −0.15 |
| 95 | Q31Y | 0.31 | 0.34 | 0.32 | −0.26 | 0.16 | −0.05 |
| 96 | D32R | 0.61 | −0.54 | 0.03 | −0.53 | −0.39 | −0.46 |
| 97 | D32E | 0.41 | 0.00 | 0.21 | −0.11 | −0.28 | −0.19 |
| 98 | D32A | 0.20 | 0.02 | 0.11 | 0.19 | 0.19 | 0.19 |
| 99 | K33H | 4.03 | 3.45 | 3.74 | 1.71 | 1.56 | 1.64 |
| 100 | K33A | 3.01 | 3.41 | 3.21 | 1.73 | 1.25 | 1.49 |
| 101 | K33C | 2.85 | 1.07 | 1.96 | 0.55 | 0.99 | 0.77 |
| 102 | K33E | 2.38 | 3.03 | 2.71 | 1.48 | 1.05 | 1.27 |
| 103 | K33I | 1.91 | 2.14 | 2.03 | 1.32 | 0.50 | 0.91 |
| 104 | K33Q | 3.03 | 2.77 | 2.90 | 1.96 | 0.99 | 1.48 |
| 105 | K33S | 2.84 | 3.22 | 3.03 | 1.34 | 1.10 | 1.22 |
| 106 | K33V | 2.71 | 2.19 | 2.45 | 2.03 | 1.46 | 1.75 |
| 107 | K33L | 2.40 | 2.65 | 2.53 | 1.67 | 1.53 | 1.60 |
| 108 | K33M | 2.30 | 2.37 | 2.34 | 1.51 | 0.61 | 1.06 |
| 109 | K33T | 1.90 | 1.63 | 1.77 | 1.48 | 1.34 | 1.41 |
| 110 | K33R | 0.73 | 0.10 | 0.42 | 0.64 | 0.10 | 0.37 |
| 111 | K33F | 1.91 | 1.62 | 1.77 | 1.16 | 0.87 | 1.02 |
| 112 | K33Y | 0.63 | 1.41 | 1.02 | 1.03 | 0.94 | 0.98 |
| 113 | K33N | 0.48 | 0.10 | 0.29 | 0.06 | 0.41 | 0.24 |
| 114 | K33W | −2.04 | 0.01 | −1.01 | 0.35 | −0.06 | 0.15 |
| 115 | E34T | 2.15 | −1.91 | 0.12 | −3.26 | −3.83 | −3.54 |
| 116 | P38L | 1.79 | 1.84 | 1.81 | 1.24 | 1.01 | 1.13 |
| 117 | P38V | 1.16 | 1.26 | 1.21 | 0.38 | −0.29 | 0.05 |
| 118 | P38S | 0.19 | 0.25 | 0.22 | 0.56 | −0.02 | 0.27 |
| 119 | P38T | 0.95 | −0.72 | 0.11 | 1.27 | 0.58 | 0.92 |
| 120 | P38C | 1.21 | 1.83 | 1.52 | 0.46 | 0.57 | 0.52 |
| 121 | P38F | 0.91 | 0.43 | 0.67 | 0.48 | 1.13 | 0.81 |
| 122 | P38W | 0.61 | 0.60 | 0.60 | −0.87 | −0.32 | −0.59 |
| 123 | P38I | 0.40 | 0.92 | 0.66 | 1.62 | −0.34 | 0.64 |
| 124 | P38A | −0.41 | 0.53 | 0.06 | −0.16 | 0.15 | 0.00 |
| 125 | P38N | 2.28 | −0.25 | 1.02 | −1.91 | −1.07 | −1.49 |
| 126 | P38Q | 0.85 | −1.35 | −0.25 | 1.18 | −0.17 | 0.50 |
| 127 | P38H | 0.87 | −0.35 | 0.26 | 0.33 | 0.83 | 0.58 |
| 128 | P38K | −2.14 | 0.10 | −1.02 | −0.61 | 1.11 | 0.25 |
| 129 | P38M | −2.07 | 1.45 | −0.31 | 1.37 | 1.13 | 1.25 |
| 130 | P38Y | 1.79 | −0.30 | 0.74 | 0.66 | −0.03 | 0.31 |
| 131 | D39Q | −3.10 | −2.46 | −2.78 | −0.45 | 0.51 | 0.03 |
| 132 | D39G | −0.20 | −0.22 | −0.21 | 0.60 | −0.49 | 0.06 |
| 133 | D39L | 0.49 | −0.38 | 0.06 | −2.90 | −1.42 | −2.16 |
| 134 | D39S | −2.04 | −0.99 | −1.51 | 0.16 | −0.08 | 0.04 |
| 135 | D39W | 0.90 | 1.09 | 0.99 | 0.86 | −0.85 | 0.00 |
| 136 | D39E | 0.29 | 0.44 | 0.36 | −0.14 | −0.23 | −0.18 |
| 137 | Q40D | 0.13 | −0.75 | −0.31 | 1.27 | 0.33 | 0.80 |
| 138 | Q40E | 1.67 | 1.08 | 1.37 | 1.84 | 0.52 | 1.18 |
| 139 | Q41V | −0.37 | 0.10 | −0.14 | 0.13 | −0.03 | 0.05 |
| 140 | Q41Y | 0.73 | 0.64 | 0.68 | 0.40 | 0.53 | 0.47 |
| 141 | Q41I | 0.30 | 0.30 | 0.30 | 0.08 | −0.25 | −0.08 |
| 142 | Q41C | 0.22 | 0.13 | 0.18 | −0.05 | 0.00 | −0.03 |
| 143 | R42S | −0.13 | −0.02 | −0.08 | 0.05 | 0.41 | 0.23 |
| 144 | R42H | 2.18 | 1.89 | 2.04 | 0.89 | 1.16 | 1.03 |
| 145 | R42F | 1.99 | 1.77 | 1.88 | 1.40 | 1.13 | 1.26 |
| 146 | R42W | 1.99 | 2.14 | 2.06 | 1.90 | 1.09 | 1.50 |
| 147 | R42Y | 1.44 | 1.69 | 1.57 | 1.13 | 1.26 | 1.19 |
| 148 | R42N | 1.18 | 1.05 | 1.12 | 1.34 | 0.68 | 1.01 |
| 149 | R42C | 0.37 | 0.47 | 0.42 | 0.54 | 0.01 | 0.28 |
| 150 | A44T | 1.70 | 0.87 | 1.28 | 0.75 | 0.59 | 0.67 |
| 151 | A46Q | 3.60 | 3.22 | 3.41 | 1.30 | 1.65 | 1.47 |
| 152 | A46G | 0.48 | 0.72 | 0.60 | 1.37 | −1.71 | −0.17 |
| 153 | K48N | −0.15 | 0.08 | −0.04 | 0.09 | 0.03 | 0.06 |
| 154 | K48T | 1.20 | 1.08 | 1.14 | 0.84 | 0.66 | 0.75 |
| 155 | K48M | 0.87 | 0.94 | 0.91 | 0.63 | 0.70 | 0.67 |
| 156 | K48V | 0.59 | 0.48 | 0.54 | 0.23 | 0.46 | 0.34 |
| 157 | K48Q | 0.59 | 0.51 | 0.55 | 0.20 | 0.47 | 0.34 |
| 158 | K48I | 0.50 | 0.77 | 0.64 | 0.35 | 0.49 | 0.42 |
| 159 | K48R | 0.39 | 0.32 | 0.35 | 0.09 | 0.19 | 0.14 |
| 160 | K48L | 0.05 | 0.04 | 0.05 | 0.28 | 0.12 | 0.20 |
| 161 | S49M | 1.00 | 0.69 | 0.84 | 0.57 | 0.98 | 0.77 |
| 162 | S49C | 0.95 | 0.24 | 0.60 | −0.04 | −0.31 | −0.18 |
| 163 | S49L | 0.85 | 1.15 | 1.00 | 0.97 | 0.80 | 0.88 |
| 164 | S49V | 0.80 | 0.23 | 0.52 | 0.52 | 0.45 | 0.49 |
| 165 | S49P | 0.65 | 0.91 | 0.78 | 0.71 | 0.44 | 0.58 |
| 166 | S49A | 0.62 | 0.39 | 0.50 | 0.76 | 0.06 | 0.41 |
| 167 | S49I | 0.04 | 0.56 | 0.30 | 0.60 | −0.26 | 0.17 |
| 168 | S49N | 0.31 | −0.23 | 0.04 | −0.59 | −0.11 | −0.35 |
| 169 | S49G | 0.27 | −0.23 | 0.02 | −0.57 | −0.05 | −0.31 |
| 170 | S49E | 0.84 | 0.04 | 0.44 | 0.96 | 0.67 | 0.81 |
| 171 | S49D | 0.11 | 0.71 | 0.41 | 0.39 | 0.49 | 0.44 |
| 172 | E51D | 0.31 | 0.72 | 0.52 | 0.29 | 0.92 | 0.61 |
| 173 | D52E | 0.43 | 0.14 | 0.28 | −0.30 | 0.37 | 0.04 |
| 174 | R54N | −0.26 | 0.65 | 0.19 | 0.03 | −0.01 | 0.01 |
| 175 | R54C | 0.24 | −0.12 | 0.06 | −0.43 | 0.29 | −0.07 |
| 176 | R54Q | −0.05 | 0.32 | 0.14 | −0.04 | 0.00 | −0.02 |
| 177 | R54F | 1.01 | 0.52 | 0.76 | 0.66 | 0.43 | 0.55 |
| 178 | R54Y | 0.92 | 0.90 | 0.91 | 0.78 | 0.71 | 0.75 |
| 179 | R54M | 0.82 | 0.89 | 0.85 | 0.56 | 0.55 | 0.56 |
| 180 | R54H | 0.78 | 0.96 | 0.87 | 0.43 | 0.55 | 0.49 |
| 181 | R54T | 0.62 | 0.71 | 0.66 | 0.76 | 0.64 | 0.70 |
| 182 | R54K | 0.07 | 0.54 | 0.30 | −0.16 | −0.29 | −0.22 |
| 183 | T55R | 0.11 | −0.22 | −0.06 | 0.19 | 0.05 | 0.12 |
| 184 | S57N | 1.72 | 0.96 | 1.34 | 0.82 | 0.56 | 0.69 |
| 185 | S57G | 1.70 | 1.63 | 1.66 | 1.24 | 0.85 | 1.05 |
| 186 | S57D | 1.05 | 1.39 | 1.22 | 0.89 | 0.83 | 0.86 |
| 187 | S57H | 0.54 | 0.90 | 0.72 | −0.04 | 0.39 | 0.17 |
| 188 | S57A | 0.29 | 0.46 | 0.37 | −0.03 | 0.08 | 0.03 |
| 189 | S57E | 0.28 | 0.62 | 0.45 | 0.42 | 0.28 | 0.35 |
| 190 | S57Q | 0.07 | 0.07 | 0.07 | 0.27 | 0.01 | 0.14 |
| 191 | S57R | 0.05 | −0.01 | 0.02 | −0.32 | −0.03 | −0.18 |
| 192 | S57K | −0.22 | −0.58 | −0.40 | −0.07 | 0.36 | 0.15 |
| 193 | S57M | −0.11 | 0.12 | 0.01 | −0.17 | 0.09 | −0.04 |
| 194 | D58S | 0.29 | 0.38 | 0.33 | 0.24 | 0.17 | 0.21 |
| 195 | N60E | 0.90 | 0.43 | 0.66 | 0.13 | 0.38 | 0.25 |
| 196 | N60Q | 0.13 | 0.03 | 0.08 | 0.01 | 0.58 | 0.29 |
| 197 | I61L | 1.10 | 1.02 | 1.06 | 0.75 | 0.59 | 0.67 |
| 198 | K63M | −0.24 | 0.09 | −0.07 | 0.18 | 0.22 | 0.20 |
| 199 | K63F | −0.01 | −0.02 | −0.02 | −0.06 | 0.22 | 0.08 |
| 200 | K63V | −0.15 | −0.11 | −0.13 | 0.00 | 0.02 | 0.01 |
| 201 | K63I | 1.39 | 1.20 | 1.29 | 0.87 | 0.70 | 0.78 |
| 202 | S65P | 3.41 | 2.89 | 3.15 | 1.91 | 1.41 | 1.66 |
| 203 | S65K | 1.61 | 1.69 | 1.65 | 0.74 | 0.53 | 0.63 |
| 204 | S65A | 1.29 | 1.01 | 1.15 | 1.17 | 0.73 | 0.95 |
| 205 | S65E | 1.29 | 1.79 | 1.54 | 0.74 | 0.85 | 0.80 |
| 206 | S65R | 1.15 | 1.13 | 1.14 | 1.48 | 0.72 | 1.10 |
| 207 | S65Q | −0.02 | 0.02 | 0.00 | 0.01 | 0.24 | 0.12 |
| 208 | S65H | 0.69 | 1.50 | 1.10 | 1.34 | 0.98 | 1.16 |
| 209 | S65N | 0.04 | 0.71 | 0.38 | 0.71 | −0.04 | 0.34 |
| 210 | S65D | 0.02 | 0.70 | 0.36 | 0.61 | 1.10 | 0.85 |
| 211 | K66R | −0.84 | −0.54 | −0.69 | 0.13 | 0.97 | 0.55 |
| 212 | L67C | −0.31 | 0.39 | 0.04 | 0.96 | 0.50 | 0.73 |
| 213 | L67Y | −0.58 | 0.86 | 0.14 | 0.50 | 0.65 | 0.58 |
| 214 | L67H | 0.84 | 1.71 | 1.27 | 3.20 | 1.87 | 2.54 |
| 215 | L67T | 0.69 | −0.52 | 0.09 | −0.30 | 0.52 | 0.11 |
| 216 | L67K | 2.08 | 1.93 | 2.01 | 1.40 | 0.39 | 0.89 |
| 217 | L67R | 1.43 | 1.74 | 1.59 | 1.05 | 0.76 | 0.90 |
| 218 | L67S | 1.15 | 1.20 | 1.18 | 1.24 | 0.63 | 0.94 |
| 219 | L67M | 0.98 | 1.07 | 1.03 | 0.65 | 0.88 | 0.77 |
| 220 | H68E | −0.69 | −1.48 | −1.08 | 0.33 | −0.13 | 0.10 |
| 221 | H68M | 2.53 | 2.04 | 2.28 | 0.99 | 1.58 | 1.28 |
| 222 | H68Q | 0.44 | −0.30 | 0.07 | −0.62 | −0.38 | −0.50 |
| 223 | P69R | −2.27 | −1.59 | −1.93 | 0.20 | 1.29 | 0.75 |
| 224 | L73M | 2.69 | 2.52 | 2.61 | 1.58 | 1.28 | 1.43 |
| 225 | R74Q | 2.60 | 1.98 | 2.29 | 1.56 | 1.52 | 1.54 |
| 226 | R74V | 1.58 | 1.44 | 1.51 | 1.19 | 0.70 | 0.95 |
| 227 | R74L | 1.35 | 0.88 | 1.11 | 0.95 | 0.76 | 0.85 |
| 228 | R74M | 1.16 | 0.91 | 1.04 | 0.68 | 0.68 | 0.68 |
| 229 | R74I | 0.84 | 0.83 | 0.83 | 0.63 | 0.58 | 0.61 |
| 230 | R74C | 0.64 | 0.99 | 0.81 | 0.68 | 0.63 | 0.65 |
| 231 | R74E | 0.53 | 0.23 | 0.38 | 0.14 | −0.35 | −0.10 |
| 232 | R74T | 0.40 | 0.26 | 0.33 | 0.25 | 0.38 | 0.31 |
| 233 | R74K | 0.04 | 0.21 | 0.12 | 0.10 | 0.17 | 0.13 |

*The amino acid substitutions highlighted in underlined, gray are also disclosed in WO2017132746A1 and are excluded as claimed subject matter herein to the extent that Ubvs that include all these amino acid substitutions (i.e., as SEQ ID NOS: 2 or 3). The reported amino acid substitutions are presented in the polypeptide amino acid sequence background of SEQ ID NO: 2 in the context of a fusion protein that includes one of the adenylate cyclase fragments.

TABLE 2
Individual screen of amino acid changes^{a}

| | | | | | Dunnett's Multiple Comparison | | |
|---|---|---|---|---|---|---|---|
| SEQ ID | A.A. | Percent GFP Positive | | | | | Adjusted |
| NO: | change | Rep 1 | Rep 2 | Rep 3 | Comparison | Summary | P Value |
| 3 | None (WT) | 9 | 7.6 | 11.8 | | | |
| 4 | M1H | 41.2 | 41.5 | 47.8 | i53 vs. | **** | <0.0001 |
| | | | | | i53 + M1H | | |
| 14 | K6R | 19.8 | 27 | 24 | i53 vs. | ** | 0.0029 |
| | | | | | i53 + K6R | | |
| 15 | T7M | 32.8 | 33.3 | 36.8 | i53 vs. | **** | <0.0001 |
| | | | | | i53 + T7M | | |
| 30 | T14E | 43.5 | 38.7 | 46.7 | i53 vs. | **** | <0.0001 |
| | | | | | i53 + T14E | | |
| 75 | V26I | 20.9 | 14.2 | 12.3 | i53 vs. | ns | 0.5657 |
| | | | | | i53 + V26I | | |
| 83 | K29M | 23.9 | 16.5 | 17.8 | i53 vs. | ns | 0.0807 |
| | | | | | i53 + K29M | | |
| 89 | Q31W | 18.3 | 8.2 | 14.6 | i53 vs. | ns | 0.9499 |
| | | | | | i53 + Q31W | | |
| 105 | K33S | 34.8 | 47 | 41.8 | i53 vs. | **** | <0.0001 |
| | | | | | i53 + K33S | | |
| 99 | K33H | 48.6 | 35.9 | 46 | i53 vs. | **** | <0.0001 |
| | | | | | i53 + K33H | | |
| 100 | K33A | 51.5 | 45.4 | 48.1 | i53 vs. | **** | <0.0001 |
| | | | | | i53 + K33A | | |
| 116 | P38L | 28.7 | 22.1 | 26.9 | i53 vs. | *** | 0.0004 |
| | | | | | i53 + P38L | | |
| 146 | R42W | 28.7 | 21.3 | 24.8 | i53 vs. | *** | 0.0009 |
| | | | | | i53 + R42W | | |
| 150 | A44T | 17.5 | 7.7 | 12.8 | i53 vs. | ns | 0.9941 |
| | | | | | i53 + A44T | | |
| 151 | A46Q | 42.6 | 26.6 | 39.1 | i53 vs. | **** | <0.0001 |
| | | | | | i53 + A46Q | | |
| 154 | K48T | 16.9 | 14 | 14.5 | i53 vs. | ns | 0.7119 |
| | | | | | i53 + K48T | | |
| 163 | S49L | 18.8 | 13.6 | 17.7 | i53 vs. | ns | 0.3845 |
| | | | | | i53 + S49L | | |
| 178 | R54Y | 21.4 | 23.8 | 20 | i53 vs. | * | 0.0142 |
| | | | | | i53 + R54Y | | |
| 185 | S57G | 31.9 | 29.9 | 25.9 | i53 vs. | **** | <0.0001 |
| | | | | | i53 + S57G | | |
| 197 | I61L | 15.9 | 17.6 | 17.2 | i53 vs. | ns | 0.3494 |
| | | | | | i53 + I61L | | |
| 201 | K63I | 50.7 | 50.9 | 52.6 | i53 vs. | **** | <0.0001 |
| | | | | | i53 + K63I | | |
| 202 | S65P | 45.8 | 39.5 | 45.5 | i53 vs. | **** | <0.0001 |
| | | | | | i53 + S65P | | |
| 216 | L67K | 24.2 | 11.2 | 21.5 | i53 vs. | ns | 0.1074 |
| | | | | | i53 + L67K | | |
| 221 | H68M | 28.6 | 23.5 | 28.3 | i53 vs. | *** | 0.0002 |
| | | | | | i53 + H68M | | |
| 224 | L73M | 36.2 | 29.2 | 39.5 | i53 vs. | **** | <0.0001 |
| | | | | | i53 + L73M | | |

^{a}NS means not significant;
*, **, ***, **** reflects qualitative measure of the strength of association the Ubv has with 53BP1 compared to the similar association of i53 with 53BP1.

### Example 2. Mutations Identified by the Two-Hybrid Screen Improve the Affinity of i53 for 53BP1 In Vitro

In order to assess the effect of mutations identified from the two-hybrid screen on the affinity of the Ubvs for 53BP1, Ubvs consisting of the i53 sequence with an N-terminal His tag and short flexible linker plus individual or combinations of screen-identified mutations were purified from E. coli (Table 3). Biolayer interferometry was used to measure the affinity of the purified proteins. Briefly, a purified Ubv was diluted in reaction buffer (1×PBS pH7.4, 0.1 mg/mL BSA, 0.001% Tween 20) to 2 ug/mL. Purified 53BP1 (amino acids 1484-1603) fused to MBP was diluted in reaction buffer to between 20 μM and 10 nM (Table 3, Table 4)). For each Ubv, 8 Ni-NTA sensor tips were hydrated and then loaded with the 2 ug/ml of a Ubv for 30 seconds. Sensor tips were then incubated in reaction buffer for 45 seconds to obtain a baseline. Tips were then moved into either empty buffer or seven different concentrations of purified 53BP1 and the association was measured. Tips were then moved back into reaction buffer and the dissociation was measured. Kon, Koff, and Kd were calculated using a 1:1 binding model using a global fit (Table 4).

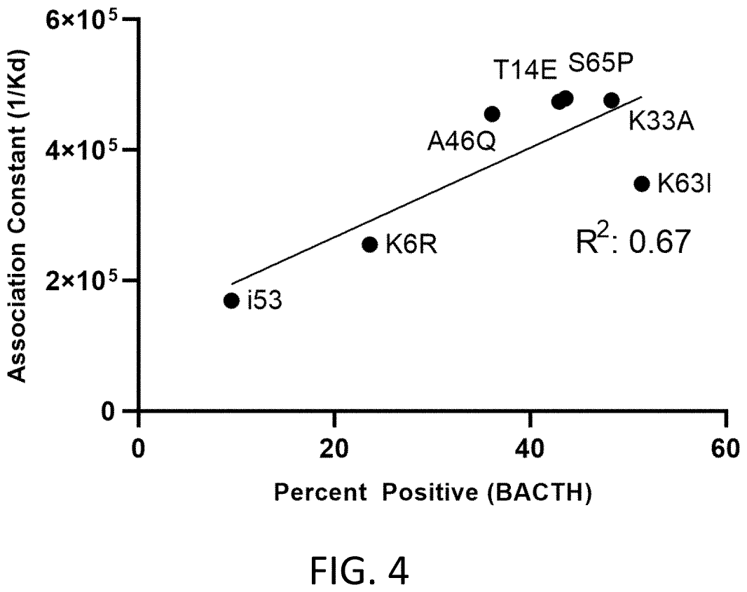

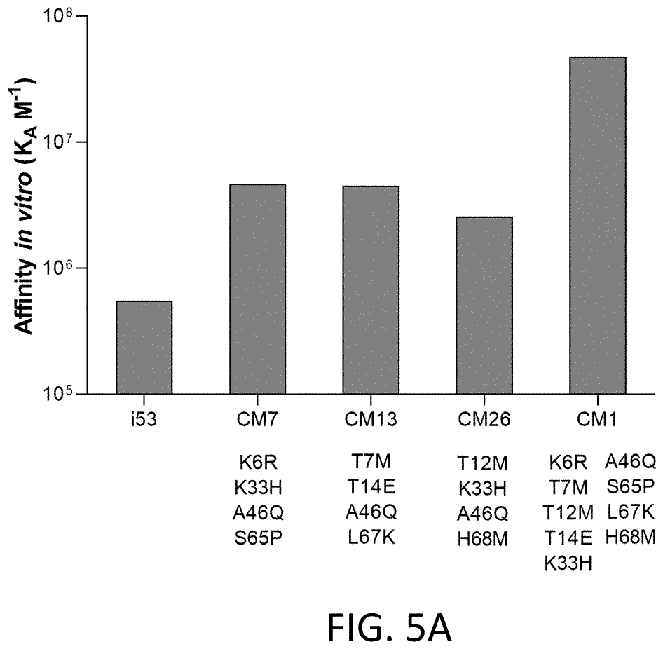

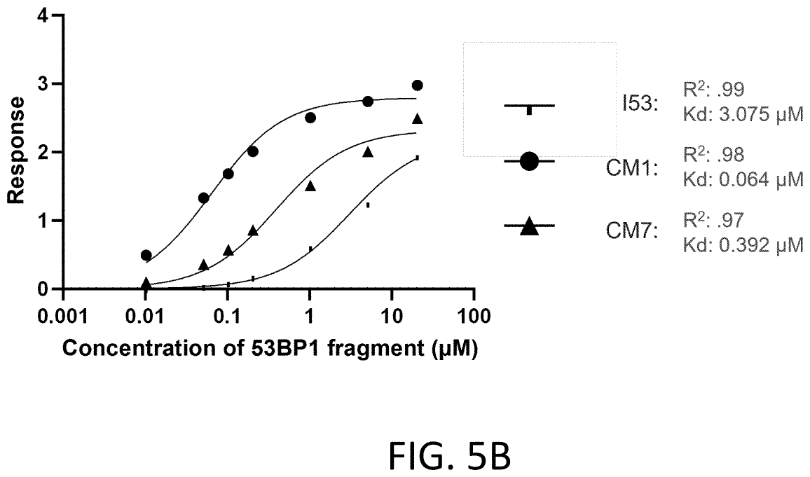

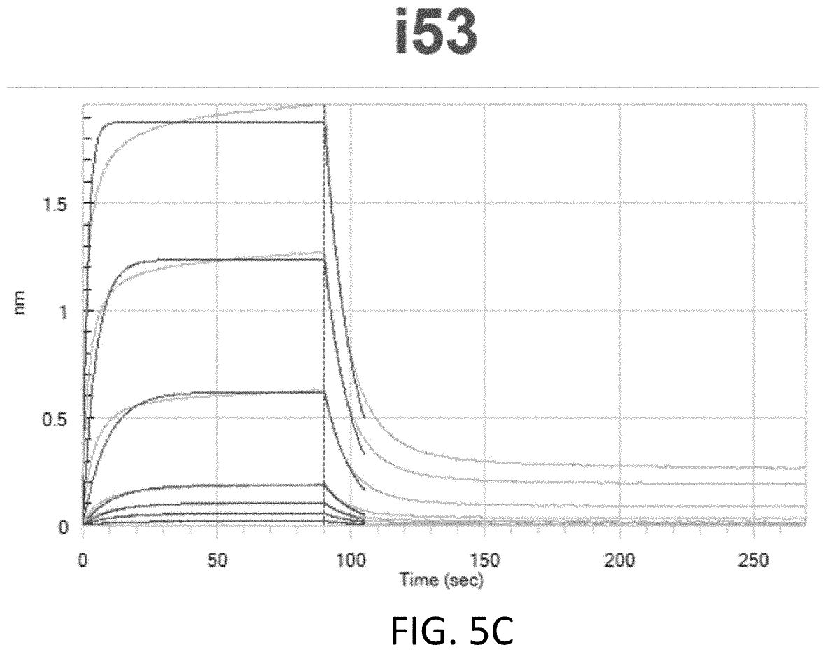

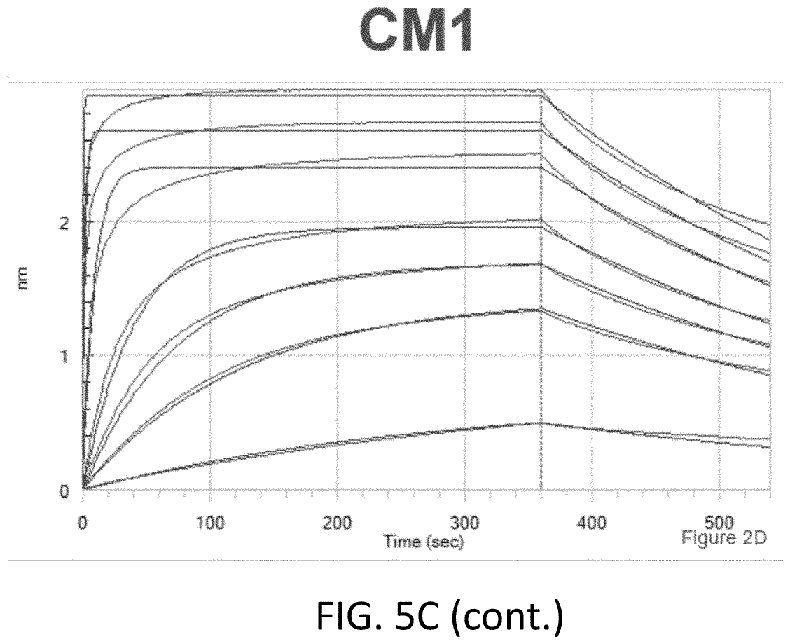

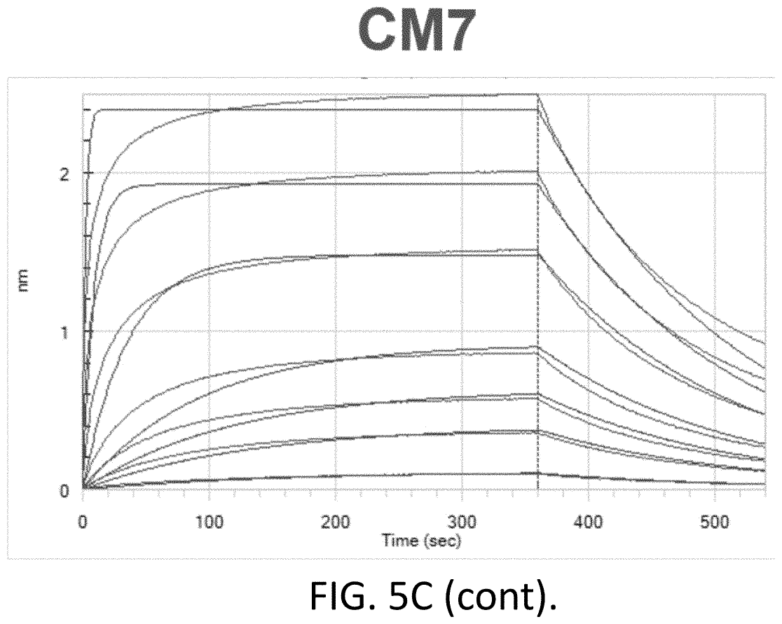

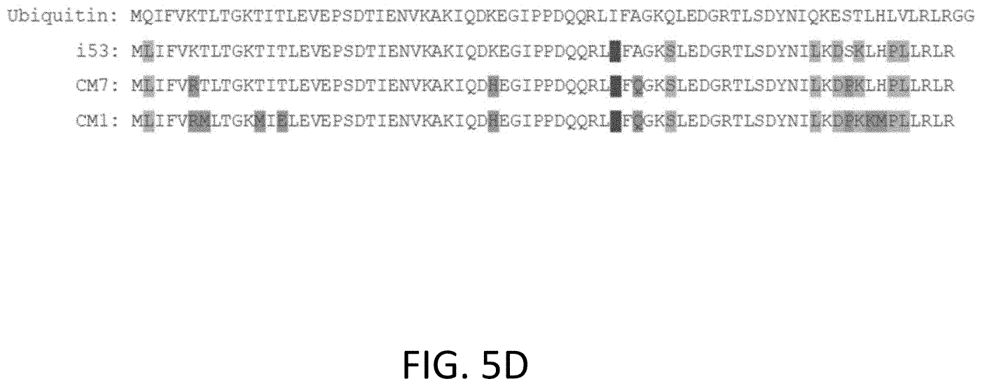

The effect of individual mutations on the affinity of the Ubv for 53BP1 was found to correlate with the percent reporter positive cells measured from the high throughput screen (FIG. 4). Ubvs containing either four or nine amino acid substitutions relative to the i53 sequence were tested using BLI and were found to have dramatically (5 to 100 fold) improved affinity for the 53BP1 fragment (FIG. 5A and Table 4). A second experiment was performed using CM1 and CM7 using a longer association time (360 seconds) to allow binding to closer approach equilibrium. The BLI response vs 53BP1 fragment concentration was plotted in prism to calculate the Kd using a one site-specific binding nonlinear fit model. An i53 response was plotted on the same graph however the association time used (90 seconds) was shorter due to needing a shorter time to reach equilibrium because of the fast off rate of i53 (FIG. 5B, FIG. 5C, Table 4).

TABLE 3
Amino acid and DNA sequences

| | Amino | | |
|---|---|---|---|
| Name | acid | | |
| [SEQ ID | changes | | |
| NOS]^{a} | in i53 | Protein Sequence | DNA sequence |
| i53 | None | MHHHHHHGGSGMLIF | ATGCACCATCACCACCACCACGGTGGAT |
| [3; 883] | | VKTLTGKTITLEVEPS | CTGGCATGTTGATTTTCGTAAAGACGTTG |
| | | DTIENVKAKIQDKEGIP | ACTGGAAAGACTATCACTTTGGAAGTGG |
| | | PDQQRLAFAGKSLED | AGCCTTCCGATACTATCGAGAATGTTAA |
| | | GRTLSDYNILKDSKLH | GGCCAAAATCCAAGATAAGGAAGGGATT |
| | | PLLRLR | CCTCCAGATCAACAACGCCTTGCTTTTGC |
| | | | CGGGAAGAGCCTGGAGGACGGTCGCAC |
| | | | ACTGTCTGACTATAACATTCTTAAAGATT |
| | | | CTAAATTGCATCCACTGCTGCGCTTGCGT |
| i53 DM | P69L, | MHHHHHHGGSGMLIF | ATGCACCATCACCACCACCACGGGGGGT |
| [234; 884] | L70V | VKTLTGKTITLEVEPS | CGGGCATGTTGATTTTCGTAAAGACGTT |
| | | DTIENVKAKIQDKEGIP | GACTGGAAAGACTATCACTTTGGAAGTG |
| | | PDQQRLAFAGKSLED | GAGCCTTCCGATACTATCGAGAATGTTA |
| | | GRTLSDYNILKDSKLH | AGGCCAAAATCCAAGATAAGGAAGGGA |
| | | LVLRLR | TTCCTCCAGATCAACAACGCCTTGCTTTT |
| | | | GCCGGGAAGAGCCTGGAGGACGGTCGC |
| | | | ACACTGTCTGACTATAACATTCTTAAAG |
| | | | ATTCTAAATTGCATCTGGTTCTGCGCTTG |
| | | | CGT |
| i53 K6R | K6R | MHHHHHHGGSGMLIF | ATGCACCATCACCACCACCACGGGGGGT |
| [235; 885] | | VRTLTGKTITLEVEPSD | CGGGCATGTTGATTTTCGTACGCACGTTG |
| | | TIENVKAKIQDKEGIPP | ACTGGAAAGACTATCACTTTGGAAGTGG |
| | | DQQRLAFAGKSLEDG | AGCCTTCCGATACTATCGAGAATGTTAA |
| | | RTLSDYNILKDSKLHP | GGCCAAAATCCAAGATAAGGAAGGGATT |
| | | LLRLR | CCTCCAGATCAACAACGCCTTGCTTTTGC |
| | | | CGGGAAGAGCCTGGAGGACGGTCGCAC |
| | | | ACTGTCTGACTATAACATTCTTAAAGATT |
| | | | CTAAATTGCATCCACTGCTGCGCTTGCGT |
| i53 T14E | T14E | MHHHHHHGGSGMLIF | ATGCACCATCACCACCACCACGGGGGGT |
| [236; 886] | | VKTLTGKTIELEVEPS | CGGGCATGTTGATTTTCGTAAAGACGTT |
| | | DTIENVKAKIQDKEGIP | GACTGGAAAGACTATCGAGTTGGAAGTG |
| | | PDQQRLAFAGKSLED | GAGCCTTCCGATACTATCGAGAATGTTA |
| | | GRTLSDYNILKDSKLH | AGGCCAAAATCCAAGATAAGGAAGGGA |
| | | PLLRLR | TTCCTCCAGATCAACAACGCCTTGCTTTT |
| | | | GCCGGGAAGAGCCTGGAGGACGGTCGC |
| | | | ACACTGTCTGACTATAACATTCTTAAAG |
| | | | ATTCTAAATTGCATCCACTGCTGCGCTTG |
| | | | CGT |
| i53 K33A | K33A | MHHHHHHGGSGMLIF | ATGCACCATCACCACCACCACGGGGGGT |
| [237; 887] | | VKTLTGKTITLEVEPS | CGGGCATGTTGATTTTCGTAAAGACGTT |
| | | DTIENVKAKIQDAEGIP | GACTGGAAAGACTATCACTTTGGAAGTG |
| | | PDQQRLAFAGKSLED | GAGCCTTCCGATACTATCGAGAATGTTA |
| | | GRTLSDYNILKDSKLH | AGGCCAAAATCCAAGATGCCGAAGGGAT |
| | | PLLRLR | TCCTCCAGATCAACAACGCCTTGCTTTTG |
| | | | CCGGGAAGAGCCTGGAGGACGGTCGCAC |
| | | | ACTGTCTGACTATAACATTCTTAAAGATT |
| | | | CTAAATTGCATCCACTGCTGCGCTTGCGT |
| i53 A46Q | A46Q | MHHHHHHGGSGMLIF | ATGCACCATCACCACCACCACGGGGGGT |
| [238; 888] | | VKTLTGKTITLEVEPS | CGGGCATGTTGATTTTCGTAAAGACGTT |
| | | DTIENVKAKIQDKEGIP | GACTGGAAAGACTATCACTTTGGAAGTG |
| | | PDQQRLAFQGKSLED | GAGCCTTCCGATACTATCGAGAATGTTA |
| | | GRTLSDYNILKDSKLH | AGGCCAAAATCCAAGATAAGGAAGGGA |
| | | PLLRLR | TTCCTCCAGATCAACAACGCCTTGCTTTT |
| | | | CAAGGGAAGAGCCTGGAGGACGGTCGC |
| | | | ACACTGTCTGACTATAACATTCTTAAAG |
| | | | ATTCTAAATTGCATCCACTGCTGCGCTTG |
| | | | CGT |
| i53 K63I | K63I | MHHHHHHGGSGMLIF | ATGCACCATCACCACCACCACGGGGGGT |
| [239; 889] | | VKTLTGKTITLEVEPS | CGGGCATGTTGATTTTCGTAAAGACGTT |
| | | DTIENVKAKIQDKEGIP | GACTGGAAAGACTATCACTTTGGAAGTG |
| | | PDQQRLAFAGKSLED | GAGCCTTCCGATACTATCGAGAATGTTA |
| | | GRTLSDYNILIDSKLHP | AGGCCAAAATCCAAGATAAGGAAGGGA |
| | | LLRLR | TTCCTCCAGATCAACAACGCCTTGCTTTT |
| | | | GCCGGGAAGAGCCTGGAGGACGGTCGC |
| | | | ACACTGTCTGACTATAACATTCTTATTGA |
| | | | TTCTAAATTGCATCCACTGCTGCGCTTGC |
| | | | GT |
| i53 S65P | S65P | MHHHHHHGGSGMLIF | ATGCACCATCACCACCACCACGGGGGGT |
| [240; 890] | | VKTLTGKTITLEVEPS | CGGGCATGTTGATTTTCGTAAAGACGTT |
| | | DTIENVKAKIQDKEGIP | GACTGGAAAGACTATCACTTTGGAAGTG |
| | | PDQQRLAFAGKSLED | GAGCCTTCCGATACTATCGAGAATGTTA |
| | | GRTLSDYNILKDPKLH | AGGCCAAAATCCAAGATAAGGAAGGGA |
| | | PLLRLR | TTCCTCCAGATCAACAACGCCTTGCTTTT |
| | | | GCCGGGAAGAGCCTGGAGGACGGTCGC |
| | | | ACACTGTCTGACTATAACATTCTTAAAG |
| | | | ATCCTAAATTGCATCCACTGCTGCGCTTG |
| | | | CGT |
| CM1 | K6R, | MHHHHHHGGSGMLIF | ATGCACCATCACCACCACCACGGTGGAT |
| [241; 916] | T7M, | VRMLTGKMIELEVEPS | CTGGCATGTTGATTTTCGTACGCATGTTG |
| | T12M, | DTIENVKAKIQDKEGIP | ACTGGAAAGATGATCGAGTTGGAAGTGG |
| | T14E, | PDQQRLAFQGKSLED | AGCCTTCCGATACTATCGAGAATGTTAA |
| | K33H, | GRTLSDYNILKDPKKM | GGCCAAAATCCAAGATCATGAAGGGATT |
| | A46Q, | PLLRLR | CCTCCAGATCAACAACGCCTTGCTTTTCA |
| | S65P, | | AGGGAAGAGCCTGGAGGACGGTCGCAC |
| | L67K, | | ACTGTCTGACTATAACATTCTTAAAGATC |
| | H68M | | CTAAAAAGATGCCACTGCTGCGCTTGCG |
| | | | T |
| CM7 | K6R, | MHHHHHHGGSGMLIF | ATGCACCATCACCACCACCACGGTGGAT |
| [242; 917] | K33H, | VRTLTGKTITLEVEPSD | CTGGCATGTTGATTTTCGTACGCACGTTG |
| | A46Q, | TIENVKAKIQDHEGIPP | ACTGGAAAGACTATCACTTTGGAAGTGG |
| | S65P | DQQRLAFQGKSLEDG | AGCCTTCCGATACTATCGAGAATGTTAA |
| | | RTLSDYNILKDPKLHP | GGCCAAAATCCAAGATCATGAAGGGATT |
| | | LLRLR | CCTCCAGATCAACAACGCCTTGCTTTTCA |
| | | | AGGGAAGAGCCTGGAGGACGGTCGCAC |
| | | | ACTGTCTGACTATAACATTCTTAAAGATC |
| | | | CTAAATTGCATCCACTGCTGCGCTTGCGT |
| CM13 | T7M, | MHHHHHHGGSGMLIF | ATGCACCATCACCACCACCACGGTGGAT |
| [243; 918] | T14E, | VKMLTGKTIELEVEPS | CTGGCATGTTGATTTTCGTAAAGATGTTG |
| | A46Q, | DTIENVKAKIQDKEGIP | ACTGGAAAGACTATCGAGTTGGAAGTGG |
| | L67K | PDQQRLAFQGKSLED | AGCCTTCCGATACTATCGAGAATGTTAA |
| | | GRTLSDYNILKDSKKH | GGCCAAAATCCAAGATAAGGAAGGGATT |
| | | PLLRLR | CCTCCAGATCAACAACGCCTTGCTTTTCA |
| | | | AGGGAAGAGCCTGGAGGACGGTCGCAC |
| | | | ACTGTCTGACTATAACATTCTTAAAGATT |
| | | | CTAAAAAGCATCCACTGCTGCGCTTGCG |
| | | | T |
| CM26 | T12M, | MHHHHHHGGSGMLIF | ATGCACCATCACCACCACCACGGTGGAT |
| [244; 919] | K33H, | VKTLTGKMITLEVEPS | CTGGCATGTTGATTTTCGTAAAGACGTTG |
| | A46Q, | DTIENVKAKIQDHEGIP | ACTGGAAAGATGATCACTTTGGAAGTGG |
| | H68M | PDQQRLAFQGKSLED | AGCCTTCCGATACTATCGAGAATGTTAA |
| | | GRTLSDYNILKDSKLM | GGCCAAAATCCAAGATCATGAAGGGATT |
| | | PLLRLR | CCTCCAGATCAACAACGCCTTGCTTTTCA |
| | | | AGGGAAGAGCCTGGAGGACGGTCGCAC |
| | | | ACTGTCTGACTATAACATTCTTAAAGATT |
| | | | CTAAATTGATGCCACTGCTGCGCTTGCGT |
| MBP | N/A | MKIEEGKLVIWINGDK | ATGAAAATCGAAGAAGGTAAACTGGTAA |
| tagged | | GYNGLAEVGKKFEKD | TCTGGATTAACGGCGATAAAGGCTATAA |
| 53BP1 | | TGIKVTVEHPDKLEEK | CGGTCTCGCTGAAGTCGGTAAGAAATTC |
| fragment | | FPQVAATGDGPDIIFW | GAGAAAGATACCGGAATTAAAGTCACCG |
| (a.a. | | AHDRFGGYAQSGLLA | TTGAGCATCCGGATAAACTGGAAGAGAA |
| 1484-1603) | | EITPDKAFQDKLYPFT | ATTCCCACAGGTTGCGGCAACTGGCGAT |
| [245; 891] | | WDAVRYNGKLIAYPI | GGCCCTGACATTATCTTCTGGGCACACG |
| | | AVEALSLIYNKDLLPN | ACCGCTTTGGTGGCTACGCTCAATCTGGC |
| | | PPKTWEEIPALDKELK | CTGTTGGCTGAAATCACCCCGGACAAAG |
| | | AKGKSALMFNLQEPY | CGTTCCAGGACAAGCTGTATCCGTTTACC |
| | | FTWPLIAADGGYAFK | TGGGATGCCGTACGTTACAACGGCAAGC |
| | | YENGKYDIKDVGVDN | TGATTGCTTACCCGATCGCTGTTGAAGCG |
| | | AGAKAGLTFLVDLIKN | TTATCGCTGATTTATAACAAAGATCTGCT |
| | | KHMNADTDYSIAEAA | GCCGAACCCGCCAAAAACCTGGGAAGA |
| | | FNKGETAMTINGPWA | GATCCCGGCGCTGGATAAAGAACTGAAA |
| | | WSNIDTSKVNYGVTV | GCGAAAGGTAAGAGCGCGCTGATGTTCA |
| | | LPTFKGQPSKPFVGVL | ACCTGCAAGAACCGTACTTCACCTGGCC |
| | | SAGINAASPNKELAKE | GCTGATTGCTGCTGACGGGGGTTATGCG |
| | | FLENYLLTDEGLEAVN | TTCAAGTATGAAAACGGCAAGTACGACA |
| | | KDKPLGAVALKSYEE | TTAAAGACGTGGGCGTGGATAACGCTGG |
| | | ELAKDPRIAATMENA | CGCGAAAGCGGGTCTGACCTTCCTGGTT |
| | | QKGEIMPNIPQMSAFW | GACCTGATTAAAAACAAACACATGAATG |
| | | YAVRTAVINAASGRQ | CAGACACCGATTACTCCATCGCAGAAGC |
| | | TVDEALKDAQTNSSSN | TGCCTTTAATAAAGGCGAAACAGCGATG |
| | | NNNNNNNNNLGIEEN | ACCATCAACGGCCCGTGGGCATGGTCCA |
| | | LYFQGHMNSFVGLRV | ACATCGACACCAGCAAAGTGAATTATGG |
| | | VAKWSSNGYFYSGKIT | TGTAACGGTACTGCCGACCTTCAAGGGT |
| | | RDVGAGKYKLLFDDG | CAACCATCCAAACCGTTCGTTGGCGTGC |
| | | YECDVLGKDILLCDPIP | TGAGCGCAGGTATTAACGCCGCCAGTCC |
| | | LDTEVTALSEDEYFSA | GAACAAAGAGCTGGCAAAAGAGTTCCTC |
| | | GVVKGHRKESGELYY | GAAAACTATCTGCTGACTGATGAAGGTC |
| | | SIEKEGQRKWYKRMA | TGGAAGCGGTTAATAAAGACAAACCGCT |
| | | VILSLEQGNRLREQYG | GGGTGCCGTAGCGCTGAAGTCTTACGAG |
| | | LG | GAAGAGTTGGCGAAAGATCCACGTATTG |
| | | | CCGCCACTATGGAAAACGCCCAGAAAGG |
| | | | TGAAATCATGCCGAACATCCCGCAGATG |
| | | | TCCGCTTTCTGGTATGCCGTGCGTACTGC |
| | | | GGTGATCAACGCCGCCAGCGGTCGTCAG |
| | | | ACTGTCGATGAAGCCCTGAAAGACGCGC |
| | | | AGACTAATTCGAGCTCGAACAACAACAA |
| | | | CAATAACAATAACAACAACCTCGGGATC |
| | | | GAGGAAAATCTGTATTTTCAGGGCCACA |
| | | | TGAATAGCTTTGTTGGTCTGCGTGTTGTT |
| | | | GCAAAATGGTCAAGCAATGGTTATTTCT |
| | | | ACAGCGGCAAAATCACCCGTGATGTTGG |
| | | | TGCAGGTAAATACAAACTGCTGTTTGAT |
| | | | GATGGTTATGAATGTGATGTGCTGGGCA |
| | | | AAGATATTCTGCTGTGTGATCCGATTCCG |
| | | | CTGGATACCGAAGTTACCGCACTGAGCG |
| | | | AAGATGAATATTTCAGTGCCGGTGTTGTT |
| | | | AAAGGCCATCGTAAAGAAAGCGGTGAA |
| | | | CTGTATTACAGCATTGAAAAAGAAGGTC |
| | | | AGCGCAAATGGTATAAACGTATGGCAGT |
| | | | TATTCTGAGCCTGGAACAGGGTAATCGT |
| | | | CTGCGTGAACAGTATGGTCTGGGT |

^{a}The SEQ ID NOS shown in brackets correspond to the protein amino acid SEQ ID NO, followed by the DNA nucleic acid SEQ ID NO.

TABLE 4
BLI Data

| | Concentration | | | | | |
|---|---|---|---|---|---|---|
| | of 53BP1 (a.a. | | | | | |
| | 1484-1603) | | | | | |
| Protein | (μM) | | | | | Full |
| (Ligand) | (Analyte) | Response | KD (M) | kon(1/Ms) | kdis(1/s) | R\hat{ }2 |
| i53 | 20 | 0.5736 | 5.92 ± 0.37E−6 | 1.50 ± 0.09E4 | 8.87 ± 0.20E−2 | 0.9867 |
| | 5 | 0.3399 | 5.92 ± 0.37E−6 | 1.50 ± 0.09E4 | 8.87 ± 0.20E−2 | 0.9867 |
| | 2 | 0.2205 | 5.92 ± 0.37E−6 | l,50 ± 0.09E4 | 8.87 ± 0.20E−2 | 0.9867 |
| | 1 | 0.1258 | 5.92 ± 0.37E−6 | 1.50 ± 0.09E4 | 8.87 ± 0.20E−2 | 0.9867 |
| | 0.5 | 0.0627 | 5.92 ± 0.37E−6 | 1.50 ± 0.09E4 | 8.87 ± 0.20E−2 | 0.9867 |
| | 0.25 | 0.0221 | 5.92 ± 0.37E−6 | 1.50 ± 0.09E4 | 8.87 ± 0.20E−2 | 0.9867 |
| | 0.125 | 0.0006 | 5.92 ± 0.37E−6 | 1.50 ± 0.09E4 | 8.87 ± 0.20E−2 | 0.9867 |
| i53 DM | 20 | 0.068 | Response was too low to get a good fit to the data | | | |
| | 5 | 0.0231 | | | | |
| | 2 | −0.0028 | | | | |
| | 1 | −0.0087 | | | | |
| | 0.5 | −0.0147 | | | | |
| | 0.25 | −0.0151 | | | | |
| | 0.125 | −0.0083 | | | | |
| i53 K6R | 20 | 0.6539 | 3.93 ± 0.23E−6 | 1.64 ± 0.09E4 | 6.44 ± 0.16E−2 | 0.9856 |
| | 5 | 0.4106 | 3.93 ± 0.23E−6 | 1.64 ± 0.09E4 | 6.44 ± 0.16E−2 | 0.9856 |
| | 2 | 0.2749 | 3.93 ± 0.23E−6 | 1.64 ± 0.09E4 | 6.44 ± 0.16E−2 | 0.9856 |
| | 1 | 0.1711 | 3.93 ± 0.23E−6 | 1.64 ± 0.09E4 | 6.44 ± 0.16E−2 | 0.9856 |
| | 0.5 | 0.0908 | 3.93 ± 0.23E−6 | 1.64 ± 0.09E4 | 6.44 ± 0.16E−2 | 0.9856 |
| | 0.25 | 0.038 | 3.93 ± 0.23E−6 | 1.64 ± 0.09E4 | 6.44 ± 0.16E−2 | 0.9856 |
| | 0.125 | 0.014 | 3.93 ± 0.23E−6 | 1.64 ± 0.09E4 | 6.44 ± 0.16E−2 | 0.9856 |
| i53 T14E | 20 | 0.6662 | 2.11 ± 0.13E−6 | 3.33 ± 0.19E4 | 7.02 ± 0.18E−2 | 0.9837 |
| | 5 | 0.4617 | 2.11 ± 0.13E−6 | 3.33 ± 0.19E4 | 7.02 ± 0.18E−2 | 0.9837 |
| | 2 | 0.333 | 2.11 ± 0.13E−6 | 3.33 ± 0.19E4 | 7.02 ± 0.18E−2 | 0.9837 |
| | 1 | 0.2242 | 2.11 ± 0.13E−6 | 3.33 ± 0.19E4 | 7.02 ± 0.18E−2 | 0.9837 |
| | 0.5 | 0.1227 | 2.11 ± 0.13E−6 | 3.33 ± 0.19E4 | 7.02 ± 0.18E−2 | 0.9837 |
| | 0.25 | 0.0571 | 2.11 ± 0.13E−6 | 3.33 ± 0.19E4 | 7.02 ± 0.18E−2 | 0.9837 |
| | 0.125 | 0.0223 | 2.11 ± 0.13E−6 | 3.33 ± 0.19E4 | 7.02 ± 0.18E−2 | 0.9837 |
| i53 K33A | 20 | 0.9597 | 2.10 ± 0.12E−6 | 2.95 ± 0.16E4 | 6.20 ± 0.16E−2 | 0.9848 |
| | 5 | 0.657 | 2.10 ± 0.12E−6 | 2.95 ± 0.16E4 | 6.20 ± 0.16E−2 | 0.9848 |
| | 2 | 0.4805 | 2.10 ± 0.12E−6 | 2.95 ± 0.16E4 | 6.20 ± 0.16E−2 | 0.9848 |
| | 1 | 0.3249 | 2.10 ± 0.12E−6 | 2.95 ± 0.16E4 | 6.20 ± 0.16E−2 | 0.9848 |
| | 0.5 | 0.1851 | 2.10 ± 0.12E−6 | 2.95 ± 0.16E4 | 6.20 ± 0.16E−2 | 0.9848 |
| | 0.25 | 0.0935 | 2.10 ± 0.12E−6 | 2.95 ± 0.16E4 | 6.20 ± 0.16E−2 | 0.9848 |
| | 0.125 | 0.0409 | 2.10 ± 0.12E−6 | 2.95 ± 0.16E4 | 6.20 ± 0.16E−2 | 0.9848 |
| i53 A46Q | 20 | 1.0136 | 2.20 ± 0.13E−6 | 2.26 ± 0.11E4 | 4.96 ± 0.14E−2 | 0.9845 |
| | 5 | 0.6996 | 2.20 ± 0.13E−6 | 2.26 ± 0.11E4 | 4.96 ± 0.14E−2 | 0.9845 |
| | 2 | 0.5003 | 2.20 ± 0.13E−6 | 2.26 ± 0.11E4 | 4.96 ± 0.14E−2 | 0.9845 |
| | 1 | 0.3476 | 2.20 ± 0.13E−6 | 2.26 ± 0.11E4 | 4.96 ± 0.14E−2 | 0.9845 |
| | 0.5 | 0.1936 | 2.20 ± 0.13E−6 | 2.26 ± 0.11E4 | 4.96 ± 0.14E−2 | 0.9845 |
| | 0.25 | 0.1021 | 2.20 ± 0.13E−6 | 2.26 ± 0.11E4 | 4.96 ± 0.14E−2 | 0.9845 |
| | 0.125 | 0.0512 | 2.20 ± 0.13E−6 | 2.26 ± 0.11E4 | 4.96 ± 0.14E−2 | 0.9845 |
| i53 K63I | 20 | 0.7969 | 2.87 ± 0.17E−6 | 1.90 ± 0.10E4 | 5.46 ± 0.15E−2 | 0.9854 |
| | 5 | 0.5263 | 2.87 ± 0.17E−6 | 1.90 ± 0.10E4 | 5.46 ± 0.15E−2 | 0.9854 |
| | 2 | 0.3744 | 2.87 ± 0.17E−6 | 1.90 ± 0.10E4 | 5.46 ± 0.15E−2 | 0.9854 |
| | 1 | 0.2422 | 2.87 ± 0.17E−6 | 1.90 ± 0.10E4 | 5.46 ± 0.15E−2 | 0.9854 |
| | 0.5 | 0.1404 | 2.87 ± 0.17E−6 | 1.90 ± 0.10E4 | 5.46 ± 0.15E−2 | 0.9854 |
| | 0.25 | 0.0623 | 2.87 ± 0.17E−6 | 1.90 ± 0.10E4 | 5.46 ± 0.15E−2 | 0.9854 |
| | 0.125 | 0.0324 | 2.87 ± 0.17E−6 | 1.90 ± 0.10E4 | 5.46 ± 0.15E−2 | 0.9854 |
| i53 S65P | 20 | 0.7157 | 2.09 ± 0.13E−6 | 2.46 ± 0.14E4 | 5.14 ± 0.16E−2 | 0.9819 |
| | 5 | 0.5076 | 2.09 ± 0.13E−6 | 2.46 ± 0.14E4 | 5.14 ± 0.16E−2 | 0.9819 |
| | 2 | 0.3612 | 2.09 ± 0.13E−6 | 2.46 ± 0.14E4 | 5.14 ± 0.16E−2 | 0.9819 |
| | 1 | 0.2516 | 2.09 ± 0.13E−6 | 2.46 ± 0.14E4 | 5.14 ± 0.16E−2 | 0.9819 |
| | 0.5 | 0.143 | 2.09 ± 0.13E−6 | 2.46 ± 0.14E4 | 5.14 ± 0.16E−2 | 0.9819 |
| | 0.25 | 0.069 | 2.09 ± 0.13E−6 | 2.46 ± 0.14E4 | 5.14 ± 0.16E−2 | 0.9819 |
| | 0.125 | 0.0384 | 2.09 ± 0.13E−6 | 2.46 ± 0.14E4 | 5.14 ± 0.16E−2 | 0.9819 |
| CM1 | 5.13 | 1.3836 | 2.10 ± 0.03E−8 | 1.47 ± 0.02E5 | 3.09 ± 0.02E−3 | 0.9826 |
| | 2.05 | 1.3075 | 2.10 ± 0.03E−8 | 1.47 ± 0.02E5 | 3.09 ± 0.02E−3 | 0.9826 |
| | 1.03 | 1.248 | 2.10 ± 0.03E−8 | 1.47 ± 0.02E5 | 3.09 ± 0.02E−3 | 0.9826 |
| | 0.5125 | 1.0736 | 2.10 ± 0.03E−8 | 1.47 ± 0.02E5 | 3.09 ± 0.02E−3 | 0.9826 |
| | 0.2562 | 0.8876 | 2.10 ± 0.03E−8 | l,47 ± 0.02E5 | 3.09 ± 0.02E−3 | 0.9826 |
| | 0.128 | 0.7242 | 2.10 ± 0.03E−8 | 1.47 ± 0.02E5 | 3.09 ± 0.02E−3 | 0.9826 |
| CM7 | 5.13 | 1.1444 | 2.14 ± 0.04E−7 | 3.33 ± 0.06E4 | 7.14 ± 0.04E−3 | 0.984 |
| | 2.05 | 0.9886 | 2.14 ± 0.04E−7 | 3.33 ± 0.06E4 | 7.14 ± 0.04E−3 | 0.984 |
| | 1.03 | 0.8003 | 2.14 ± 0.04E−7 | 3.33 ± 0.06E4 | 7.14 ± 0.04E−3 | 0.984 |
| | 0.5125 | 0.5888 | 2.14 ± 0.04E−7 | 3.33 ± 0.06E4 | 7.14 ± 0.04E−3 | 0.984 |
| | 0.2562 | 0.4015 | 2.14 ± 0.04E−7 | 3.33 ± 0.06E4 | 7.14 ± 0.04E−3 | 0.984 |
| | 0.128 | 0.2514 | 2.14 ± 0.04E−7 | 3.33 ± 0.06E4 | 7.14 ± 0.04E−3 | 0.984 |
| CM13 | 5.13 | 1.3261 | 2.22 ± 0.04E−7 | 4.07 ± 0.07E4 | 9.02 ± 0.05E−3 | 0.9863 |
| | 2.05 | 1.1469 | 2.22 ± 0.04E−7 | 4.07 ± 0.07E4 | 9.02 ± 0.05E−3 | 0.9863 |
| | 1.03 | 0.9475 | 2.22 ± 0.04E−7 | 4.07 ± 0.07E4 | 9.02 ± 0.05E−3 | 0.9863 |
| | 0.5125 | 0.6938 | 2.22 ± 0.04E−7 | 4.07 ± 0.07E4 | 9.02 ± 0.05E−3 | 0.9863 |
| | 0.2562 | 0.4733 | 2.22 ± 0.04E−7 | 4.07 ± 0.07E4 | 9.02 ± 0.05E−3 | 0.9863 |
| | 0.128 | 0.3065 | 2.22 ± 0.04E−7 | 4.07 ± 0.07E4 | 9.02 ± 0.05E−3 | 0.9863 |
| CM26 | 5.13 | 1.0663 | 1.23 ± 0.05E−7 | 1.36 ± 0.05E5 | 1.67 ± 0.02E−2 | 0.9642 |
| | 2.05 | 0.9555 | 1.23 ± 0.05E−7 | 1.36 ± 0.05E5 | 1.67 ± 0.02E−2 | 0.9642 |
| | 1.03 | 0.821 | 1.23 ± 0.05E−7 | 1.36 ± 0.05E5 | 1.67 ± 0.02E−2 | 0.9642 |
| | 0.5125 | 0.6303 | 1.23 ± 0.05E−7 | 1.36 ± 0.05E5 | 1.67 ± 0.02E−2 | 0.9642 |
| | 0.2562 | 0.4422 | 1.23 ± 0.05E−7 | 1.36 ± 0.05E5 | 1.67 ± 0.02E−2 | 0.9642 |
| | 0.128 | 0.298 | 1.23 ± 0.05E−7 | 1.36 ± 0.05E5 | 1.67 ± 0.02E−2 | 0.9642 |
| CM1 - longer | 20.5 | 2.9739 | 2.35 ± 0.02E−8 | 1.08 ± 0.01E5 | 2.54 ± 0.01E−3 | 0.9939 |
| association | 5.11 | 2.738 | 2.35 ± 0.02E−8 | 1.08 ± 0.01E5 | 2.54 ± 0.01E−3 | 0.9939 |
| | 1.02 | 2.5002 | 2.35 ± 0.02E−8 | 1.08 ± 0.01E5 | 2.54 ± 0.01E−3 | 0.9939 |
| | 0.2045 | 2.0092 | 2.35 ± 0.02E−8 | 1.08 ± 0.01E5 | 2.54 ± 0.01E−3 | 0.9939 |
| | 0.1022 | 1.6825 | 2.35 ± 0.02E−8 | 1.08 ± 0.01E5 | 2.54 ± 0.01E−3 | 0.9939 |
| | 0.0511 | 1.3298 | 2.35 ± 0.02E−8 | 1.08 ± 0.01E5 | 2.54 ± 0.01E−3 | 0.9939 |
| | 0.0102 | 0.4913 | 2.35 ± 0.02E−8 | 1.08 ± 0.01E5 | 2.54 ± 0.01E−3 | 0.9939 |
| CM7 - longer | 20.5 | 2.4923 | 2.97 ± 0.05E−7 | 2.15 ± 0.04E4 | 6.38 ± 0.04E−3 | 0.99 |
| association | 5.11 | 2.0067 | 2.97 ± 0.05E−7 | 2.15 ± 0.04E4 | 6.38 ± 0.04E−3 | 0.99 |
| | 1.02 | 1.5108 | 2.97 ± 0.05E−7 | 2.15 ± 0.04E4 | 6.38 ± 0.04E−3 | 0.99 |
| | 0.2045 | 0.8611 | 2.97 ± 0.05E−7 | 2.15 ± 0.04E4 | 6.38 ± 0.04E−3 | 0.99 |
| | 0.1022 | 0.5715 | 2.97 ± 0.05E−7 | 2.15 ± 0.04E4 | 6.38 ± 0.04E−3 | 0.99 |
| | 0.0511 | 0.3578 | 2.97 ± 0.05E−7 | 2.15 ± 0.04E4 | 6.38 ± 0.04E−3 | 0.99 |
| | 0.0102 | 0.099 | 2.97 ± 0.05E−7 | 2.15 ± 0.04E4 | 6.38 ± 0.04E−3 | 0.99 |
| i53 - matched | 20.5 | 1.954 | 3.87 ± 0.14E−6 | 2.30 ± 0.08E4 | 8.92 ± 0.13E−2 | 0.9956 |
| dosage range | 5.11 | 1.2658 | 3.87 ± 0.14E−6 | 2.30 ± 0.08E4 | 8.92 ± 0.13E−2 | 0.9956 |
| for longer | 1.02 | 0.6247 | 3.87 ± 0.14E−6 | 2.30 ± 0.08E4 | 8.92 ± 0.13E−2 | 0.9956 |
| association | 0.2045 | 0.1877 | 3.87 ± 0.14E−6 | 2.30 ± 0.08E4 | 8.92 ± 0.13E−2 | 0.9956 |
| experiment | 0.1022 | 0.104 | 3.87 ± 0.14E−6 | 2.30 ± 0.08E4 | 8.92 ± 0.13E−2 | 0.9956 |
| | 0.0511 | 0.0537 | 3.87 ± 0.14E−6 | 2.30 ± 0.08E4 | 8.92 ± 0.13E−2 | 0.9956 |
| | 0.0102 | 0.0176 | 3.87 ± 0.14E−6 | 2.30 ± 0.08E4 | 8.92 ± 0.13E−2 | 0.9956 |

### Example 3. Ubvs with Higher Affinity for 53BP1 than i53 are More Effective at Improving Rates of HDR

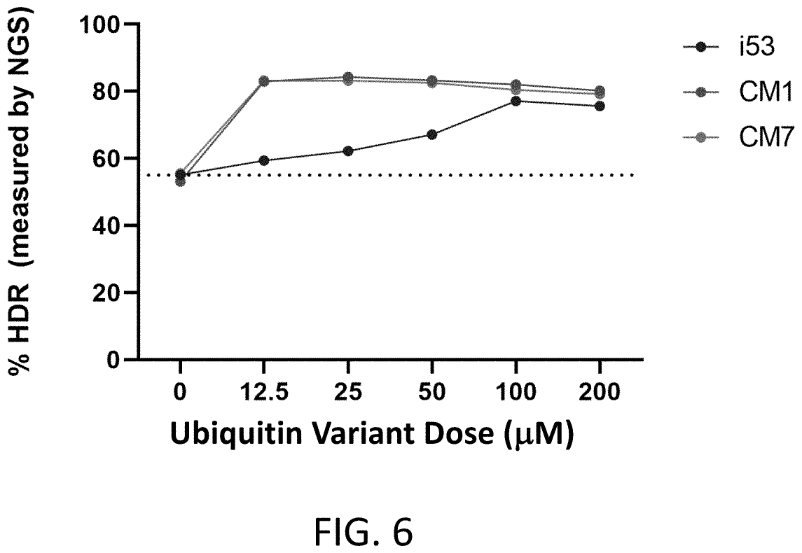

In order to test the effects of the improved affinity of the combination mutant Ubvs for 53BP1 on HIDR, i53, CM1, and CM7 Ubvs were purified and used for testing in human cells (Table 3). The Ubvs were delivered alongside Cas9 V3 (JDT) RNP targeting a site in SERPINC1 with single stranded Alt-R HIDR Donor Oligoes (JDR) to introduce an EcoR1 cut site sequence (GAATTC) at the Cas9 cut site upon successful HIDR (Table 5, see methods described below). A range of Ubvs doses was tested from 12.5 to 200 μM. The improved affinity ubiquitin variants required ˜10 fold lower dose for maximum effectiveness and the HDR rates were improved beyond what could be achieved with the i53 peptide (FIG. 6).

TABLE 5
Guide and donor information

| | | Proto- | | | |
|---|---|---|---|---|---|
| | | spacer | | | ssODN |
| | coor- | [SEQ | | Guide | sequence |
| | di- | ID | coor- | sequence | [SEQ ID |
| | nates | NO: | di- | [SEQ ID | NO: |
| Gene | (hg38) | 1101] | nates | NO: 1102] | 1103] |
| SERPINCI | chr1: | ACCTCTG | chr1: | mA*mC*mC*r | /Alt-R- |
| | 173, | GAAAAAG | 173, | UrCrUrGrGr | HDR1/A* |
| | 903, | GTAAGA | 917, | ArArArArAr | T*TCCAA |
| | 800- | | 213- | GrGrUrArAr | TGTGATA |
| | 173, | | 173, | GrArGrUrUr | GGAACTG |
| | 917, | | 917, | UrUrArGrAr | TAACCTC |
| | 327 | | 232 | GrCrUrArGr | TGGAAAA |
| | | | | ArArArUrAr | AGGTAGA |
| | | | | GrCrArArGr | ATTCAGA |
| | | | | UrUrArArAr | GGGGTGA |
| | | | | ArUrArArGr | GCTTTCC |
| | | | | GrCrUrArGr | CCTTGCC |
| | | | | UrCrCrGrUr | TGCCCCT |
| | | | | UrArUrCrAr | ACTGGGT |
| | | | | ArCrUrUrGr | *T*T/ |
| | | | | ArArArArAr | Alt-R- |
| | | | | GrUrGrGrCr | HDR2/ |
| | | | | ArCrCrGrAr | |
| | | | | GrUrCrGrGr | |
| | | | | UrGrCmU*mU | |
| | | | | *mU*rU | |

Genome editing was mediated via IDT Alt-R Cas9 ribonucleoprotein (RNP) complexes delivered by Lonza nucleofection in concert with single-stranded oligodeoxynucleotide (ssODN) HDR repair templates. The specific repair event was the insertion of the 6-nt EcoR1 sequence (5′-GAATTC-3′) directly at the canonical Sp Cas9 cut site (between bases 3 and 4 in the 5′-direction from the PAM sequence). HDR complexes were formed with a nuclease-specific guide for the SERPINC1 gene (Table 5). HDR template consisted of a chemically modified ssODN synthesized as IDT Alt-R HDR Donor Oligos with the Alt-R modification. The sequence contains 40-nt homology arms (HA) on the 5′-end, the 6-nt EcoR1 sequence in the center of the oligo and 40-nt HA on the 3′-end (Table 5). The 86-nt repair template was homologous to the non-targeting strand of dsDNA, where targeting/non-targeting is defined with respect to the guide RNA sequence and the presence of the PAM sequence identifying the targeting strand. The RNPs were generated by complexing IDT Alt-R Cas9 to IDT Alt-R sgRNA at a 1:1.2 ratio of protein to guide to give a final concentration of 2 uM Cas9 with 2.4 uM guide RNA where final concentration refers to the concentration in the final cells, protein, RNA, and DNA mix. The Ubv protein was added to the Cas9 RNP at varying amounts (200 μM down to 12.5 μM final concentration) along with donor DNA at a final concentration of 2 uM. Cas9 RNP, donor, and Ubv protein was delivered into HEK293 cells using the Lonza 96-well Shuttle and nucleofection protocol 96-DS-150. The cells were allowed to grow for 48 hours, after which genomic DNA was isolated using QuickExtract (Epicentre). HDR was measured by NGS.

### Example 4. Additional Stacking of Screen-Identified Mutations Resulted in the Generation of Ubiquitin Variants with Improved In Vitro Affinity for 53BP1 Relative to i53 that do not Contain any of the Original i53 Mutations

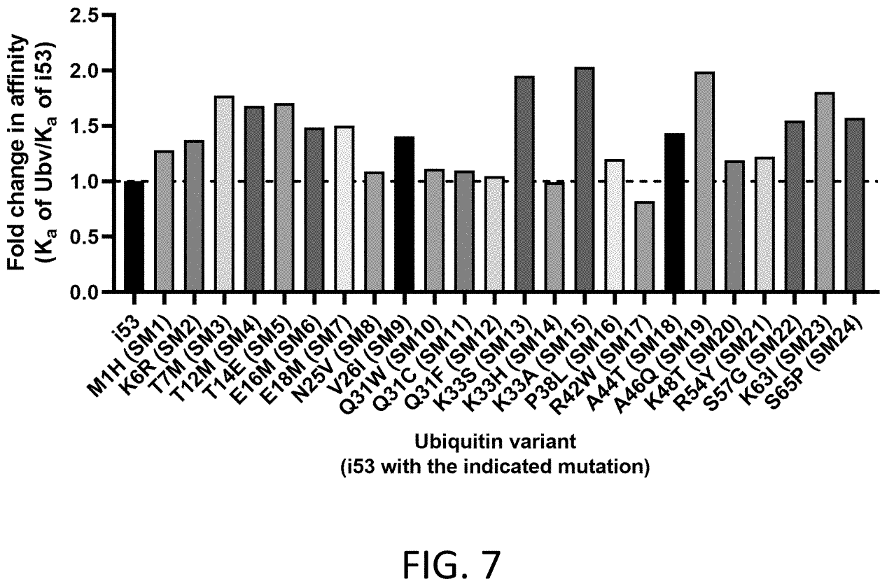

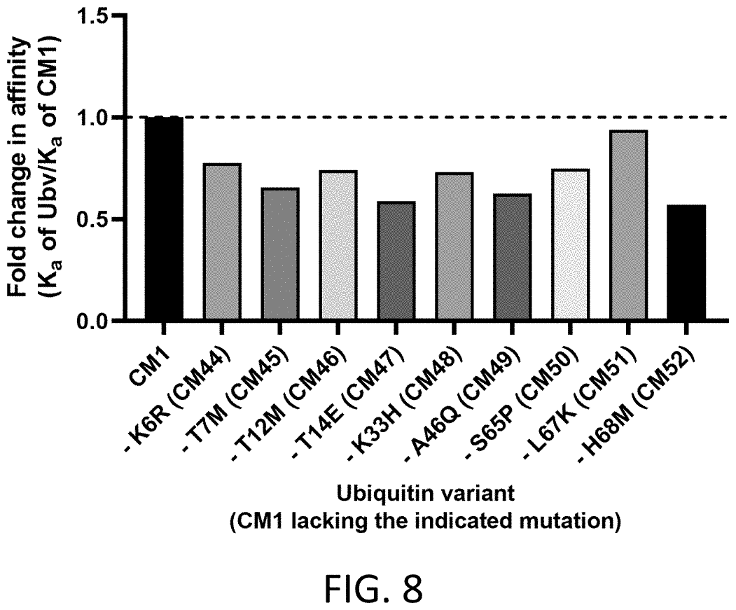

Testing of additional combinations of mutations identified variants with improved affinity over the previous best variant, CM1. In order to further validate the amino acids changes identified in the two-hybrid screen as candidates for improving the affinity of our Ubvs for 53BP1, a subset of the top hits from the screen were individually added to i53, the results of this screen are shown in FIG. 7. For graphs in this invention disclosure labeled as “Fold change in affinity”, affinity is graphed as the association constant (KA) of the ubiquitin variant being tested divided by the KA of the reference ubiquitin variant, typically the base construct upon which further mutations are stacked as determined by calculating each affinity for binding a fragment of 53BP1 (Table 6) using biolayer interferometry (BLI). The BLI steady-state response versus 53BP1 fragment concentration was plotted in prism to calculate the Kd using a one site-specific binding nonlinear fit model. If the affinity of a ubiquitin variant being tested is higher (binding is tighter) than for the reference ubiquitin variant, then the fold change in affinity will be >1. Of the mutations tested, the majority were shown to result in improved affinity (fold change >1) relative to i53, indicating that positive hits from two-hybrid screen reliably identified mutations that improved affinity. In order to validate if CM1 was the best starting combination of mutations for additional stacking, the contribution of each of the 9 mutations present in CM1 relative to i53 was analyzed and is shown in FIG. 8. Loss of any of the mutations resulted in reduced affinity indicating that each mutation contributes to the overall affinity of CM1 for binding 53BP1. Additional mutations were then added to CM1 either alone or in combination to determine if the affinity could be further improved.

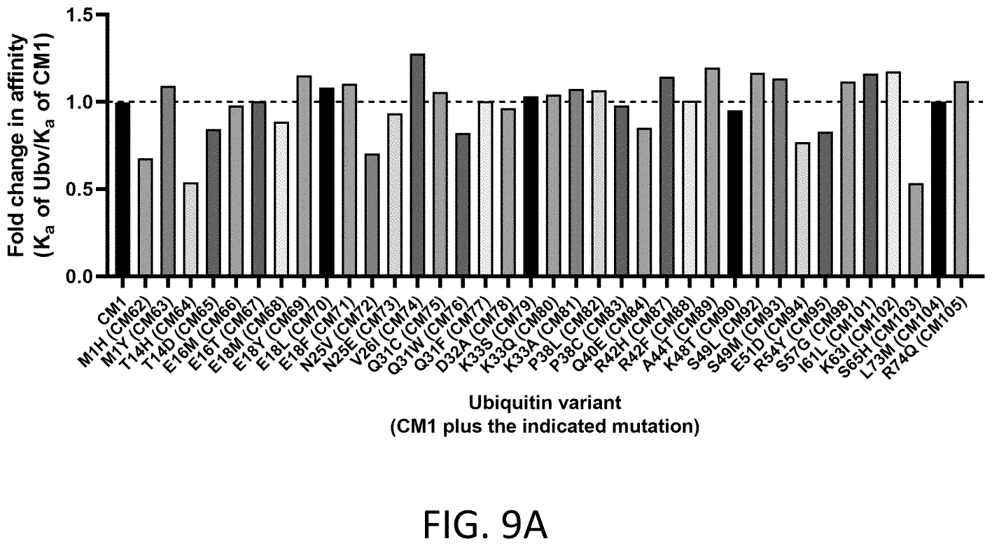

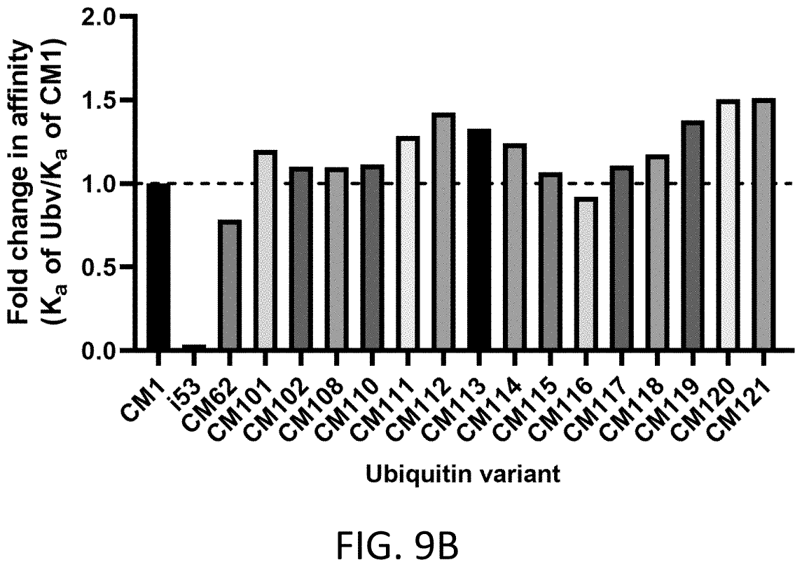

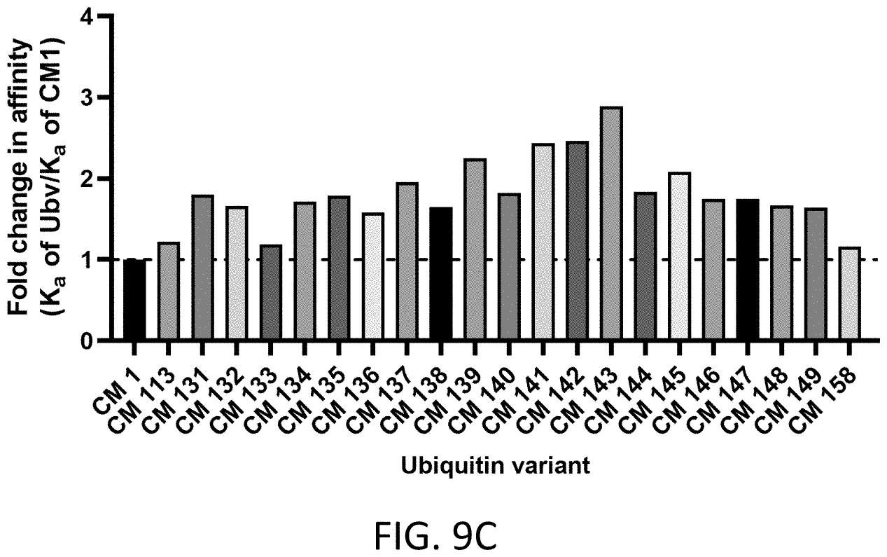

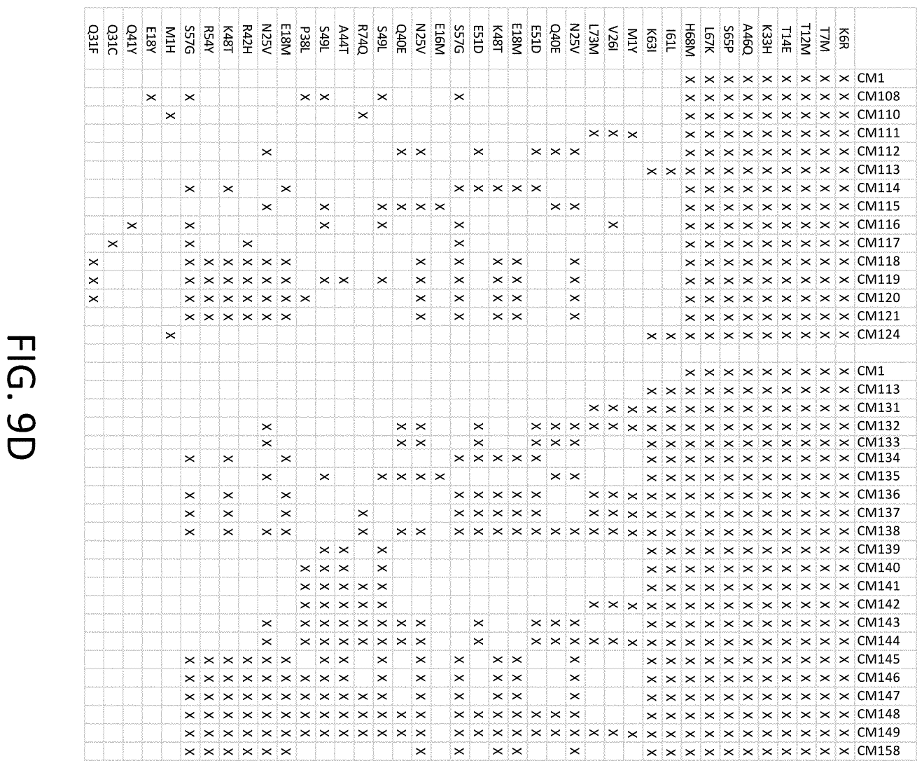

The results of that experiment are shown in FIG. 9. Many individual and combinations of mutations were identified that improve the affinity of CM1 for 53BP1 (FIGS. 9A and 9B) with the best individual mutations improving affinity by approximately 25%. Subsequent combining of the groups of mutations or parts of the groups of mutations identified as beneficial resulted in ubiquitin variants with a further benefit to affinity (FIG. 9B), with maximal benefit being an approximately 50% improvement in affinity over CM1. Subsequent additional stacking identified combinations of mutations that provided a 2-3 fold benefit to affinity over CM1 (FIG. 9C). Notably, the combinations of (M1Y, V26I, L73M-CM131), (E18M, K48T, E51D, S57G-CM134), (E16M, N25V, Q40E, S49L-CM135), (R74Q-comparison of CM136 to CM137 and CM140 to CM141), and (A44T, S49L-CM139) were notably beneficial when added to a base of CM113. All of the combinations tested had improved affinity over CM1.

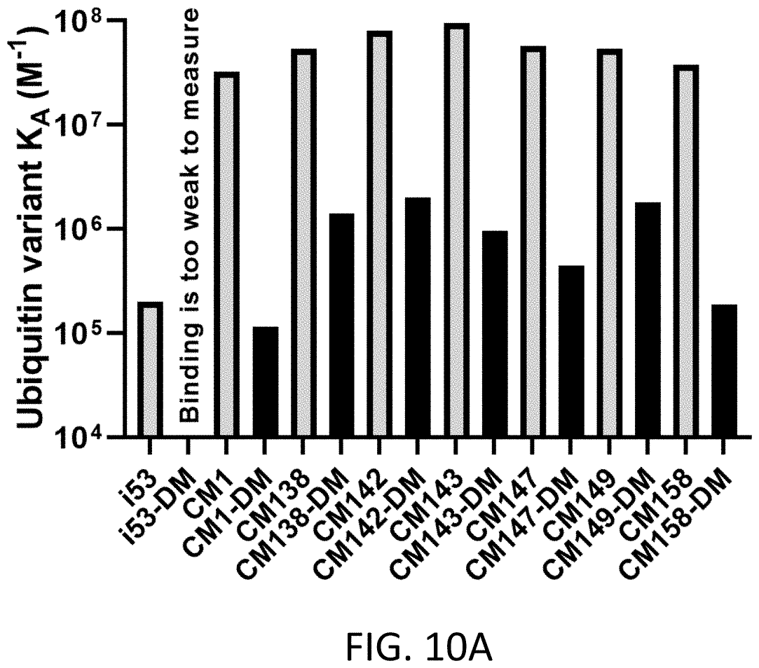

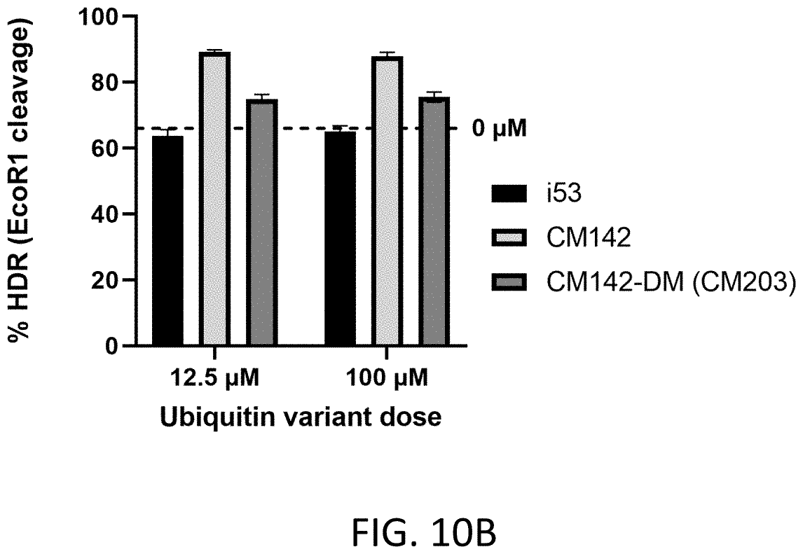

To narrow down which variant may have the best activity in cells CM138, CM142, CM143, CM147, CM149, CM158 were selected for additional testing. The 53BP1-binding deficiency mutant amino acid substitutions (P69L and L70V) were added to CM142, CM143, CM147, CM149, and CM158 and the effect on affinity was measured using BLI¹¹. The results are shown in FIG. 10, with CM142 having the best tolerance for the DM mutations. CM142 and CM142-DM (CM203) were also tested for their ability to improve the rate of HDR in cells (FIG. 10B). CM142 was found to provide a significantly increased benefit to HDR over i53. Further, CM142-DM, despite having the mutations that eliminate i53 binding to 53BP1, also showed an improved benefit to HDR over i53.

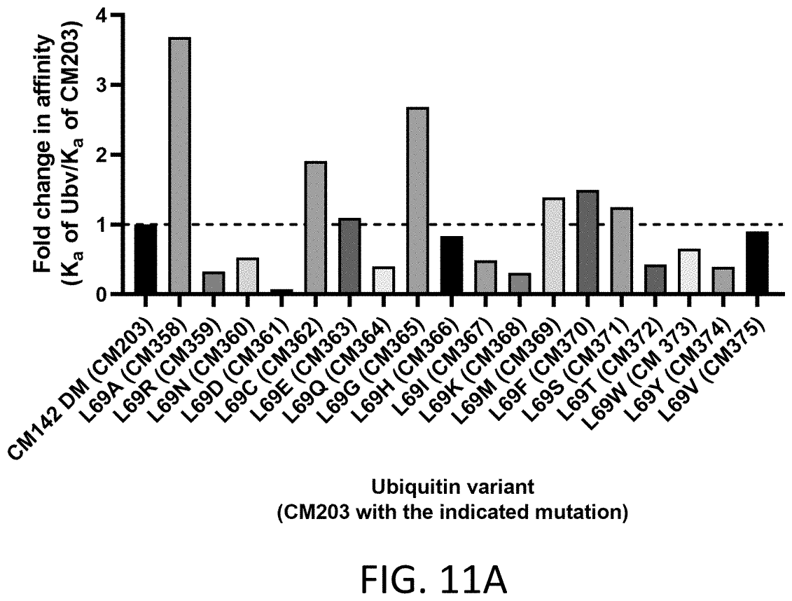

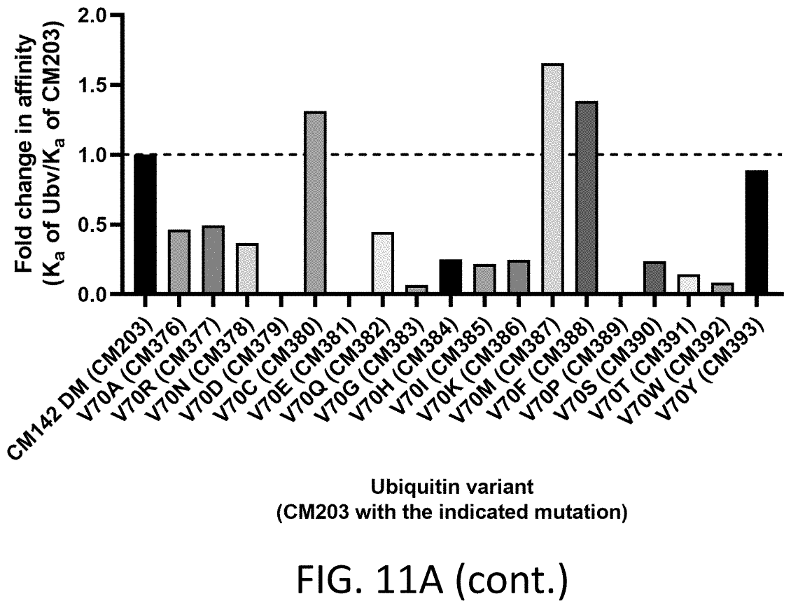

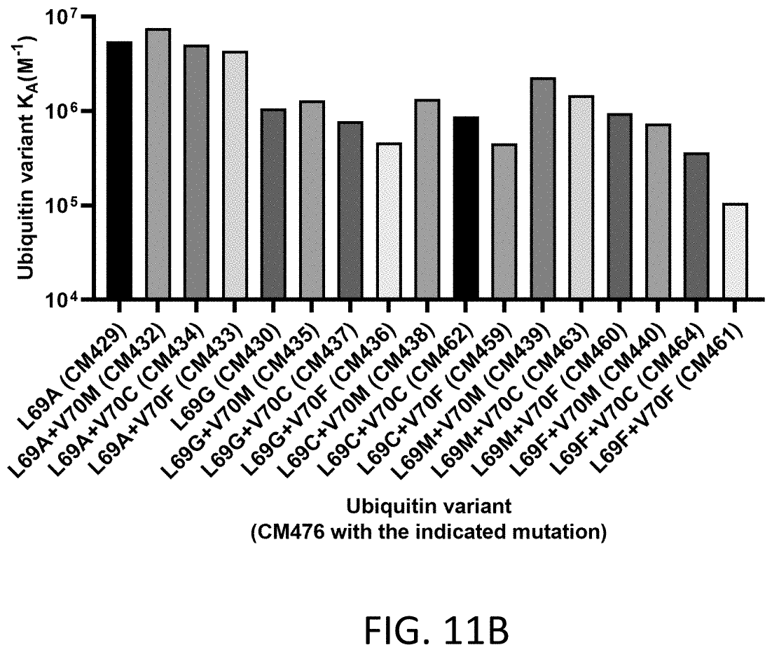

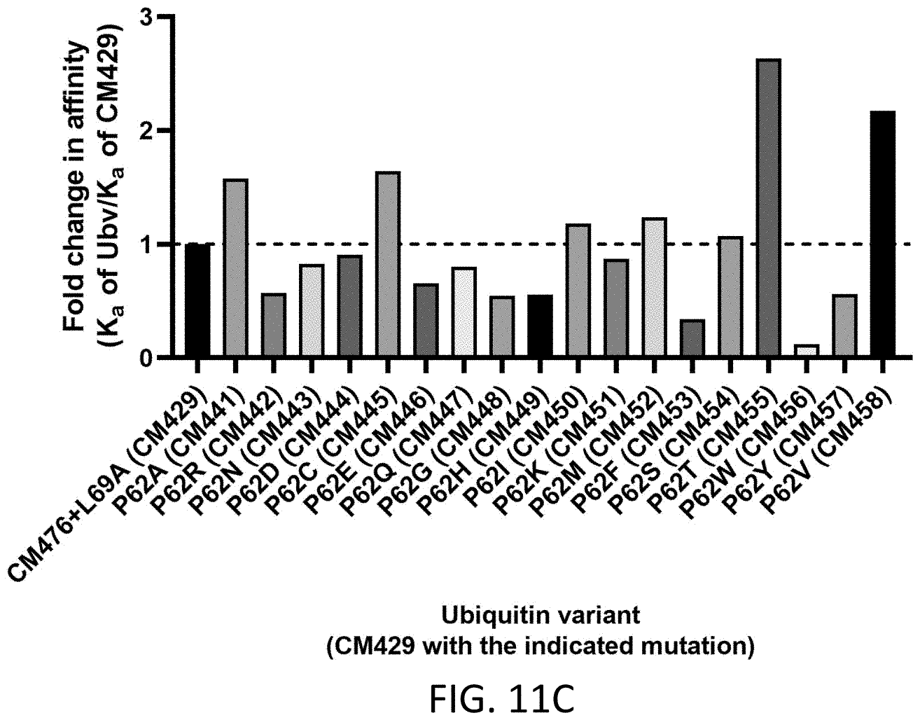

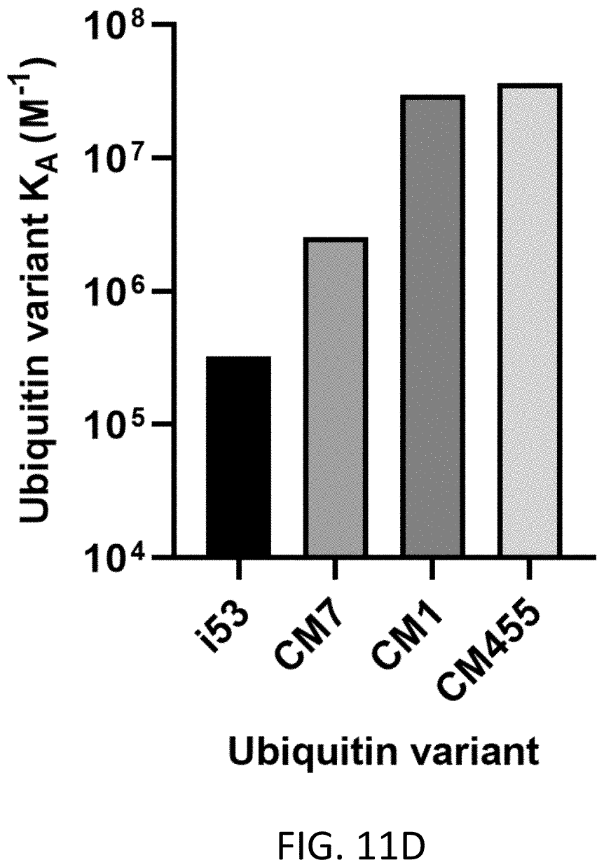

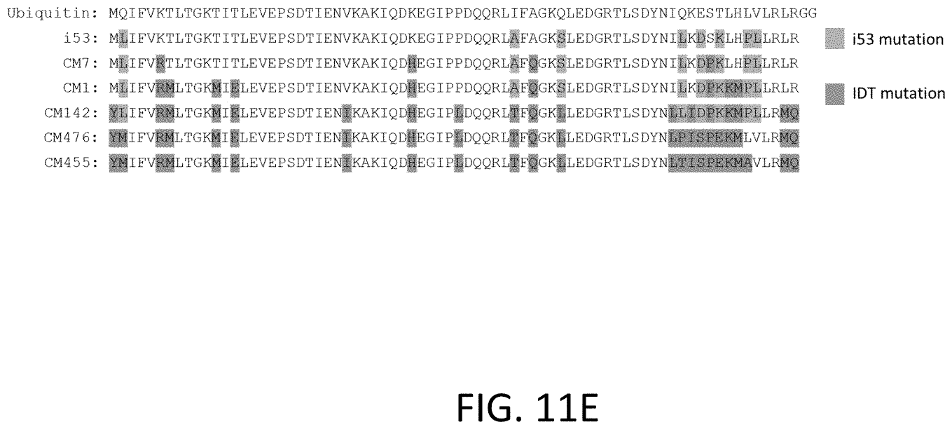

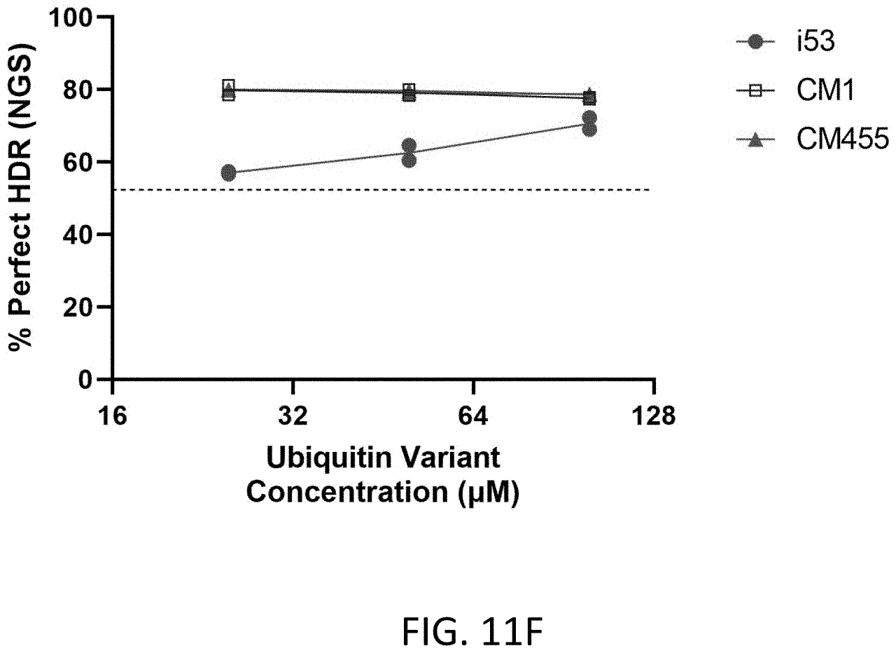

Screening of possible alternative mutations at positions mutated in i53 resulted in the identification of high affinity ubiquitin variants that do not include any of the mutations present in i53. Given the tolerance of CM142 for the DM mutations (FIG. 10A), additional screening was performed at positions 62, 69, and 70 to identify alternative beneficial amino acids at those positions. A screen was conducted using CM142-DM (CM203) as the base construct and positions 69 or 70 were individually mutated to the 18 amino acids not present in i53 or wildtype ubiquitin. The results are shown in FIG. 11A. For position 69, 69A and 69G were most beneficial. For position 70, 70M, 70F and 70C were most beneficial. The only i53 mutations remaining in CM142 DM are Q2L, Q62L, E64D, and T66K relative to wild-type ubiquitin (FIG. 11E). From our two-hybrid screen L2M, L62P, D64S, and K66E were identified as providing the second-best benefit to affinity relative to the published mutations in i53 at those positions (data not shown). L2M, L62P, D64S, and K66E mutations were added to CM142 DM and this variant (CM476-FIG. 11E) was used as a baseline construct for testing combinations of DM position mutations. Further, CM476+L69A (CM429) was used to screen all possible alternatives at position 62 since Q62P was a poor alternative to Q62L (relative to wildtype ubiquitin) based on the two-hybrid screen. The result of this screening is shown in FIGS. 11B and 11C. Relative to CM142 DM, L69A+V70M was identified as the most beneficial combination of mutations at positions 69 and 70, and A, C, T, and V were identified as the most beneficial amino acids at position 62. Together, these data indicate that some combination of CM142 DM plus L69A+V70M and either P62A, P62C, P62T, or P62V (CM465, CM467, CM468, and CM469 in Table 6) relative to CM476 will result in a variant containing no i53 mutations with the best affinity for 53BP1. The V70M mutation was found to affect purification (data not shown), so CM455 (containing the P62T and L69A mutations relative to CM476, FIG. 11E) was selected for further testing. The affinity CM455, CM1, and i53 for binding a fragment of 53BP1 as measured by BLI is shown in FIG. 11D. The affinity of CM455 for binding 53BP1 is on par with or slightly better than that of CM1, despite having none of the amino acid changes present in i53 relative to wildtype ubiquitin other than removal of the terminal glycine residues.

To determine if CM455 is able to enhance rates of HDR, we tested its ability to improve rates of HDR measured by introduction of an EcoR1 cut site sequence at SERPINC1 as described in Example 3 with the exception that editing was measured using next generation sequencing. The results are shown in FIG. 11F. CM455 was able to boost HDR rates to higher levels and at lower concentrations than i53.

TABLE 6
Amino acid and DNA sequences described in Example 4

| | Amino acid | | |
|---|---|---|---|
| Name | changes | | |
| [SEQ ID | relative | Protein | |
| NOS]^{a} | to i53 | Sequence | DNA sequence |
| i53 | None | MHHHHHHGGS | ATGCACCATCACCACCACCACGGTGGATCTG |
| [3; 883] | | GMLIFVKTLTGKT | GCATGTTGATTTTCGTAAAGACGTTGACTGGA |
| | | ITLEVEPSDTIENV | AAGACTATCACTTTGGAAGTGGAGCCTTCCG |
| | | KAKIQDKEGIPPD | ATACTATCGAGAATGTTAAGGCCAAAATCCA |
| | | QQRLAFAGKSLE | AGATAAGGAAGGGATTCCTCCAGATCAACAA |
| | | DGRTLSDYNILKD | CGCCTTGCTTTTGCCGGGAAGAGCCTGGAGG |
| | | SKLHPLLRLR | ACGGTCGCACACTGTCTGACTATAACATTCTT |
| | | | AAAGATTCTAAATTGCATCCACTGCTGCGCTT |
| | | | GCGT |
| i53 DM | P69L, L70V | MHHHHHHGGS | ATGCACCATCACCACCACCACGGGGGGTCGG |
| [234; 884] | | GMLIFVKTLTGKT | GCATGTTGATTTTCGTAAAGACGTTGACTGGA |
| | | ITLEVEPSDTIENV | AAGACTATCACTTTGGAAGTGGAGCCTTCCG |
| | | KAKIQDKEGIPPD | ATACTATCGAGAATGTTAAGGCCAAAATCCA |
| | | QQRLAFAGKSLE | AGATAAGGAAGGGATTCCTCCAGATCAACAA |
| | | DGRTLSDYNILKD | CGCCTTGCTTTTGCCGGGAAGAGCCTGGAGG |
| | | SKLHLVLRLR | ACGGTCGCACACTGTCTGACTATAACATTCTT |
| | | | AAAGATTCTAAATTGCATCTGGTTCTGCGCTT |
| | | | GCGT |
| i53 K6R | K6R | MHHHHHHGGS | ATGCACCATCACCACCACCACGGGGGGTCGG |
| [235; 885] | | GMLIFVRTLTGKT | GCATGTTGATTTTCGTACGCACGTTGACTGGA |
| | | ITLEVEPSDTIENV | AAGACTATCACTTTGGAAGTGGAGCCTTCCG |
| | | KAKIQDKEGIPPD | ATACTATCGAGAATGTTAAGGCCAAAATCCA |
| | | QQRLAFAGKSLE | AGATAAGGAAGGGATTCCTCCAGATCAACAA |
| | | DGRTLSDYNILKD | CGCCTTGCTTTTGCCGGGAAGAGCCTGGAGG |
| | | SKLHPLLRLR | ACGGTCGCACACTGTCTGACTATAACATTCTT |
| | | | AAAGATTCTAAATTGCATCCACTGCTGCGCTT |
| | | | GCGT |
| i53 T14E | T14E | MHHHHHHGGS | ATGCACCATCACCACCACCACGGGGGGTCGG |
| [236; 886] | | GMLIFVKTLTGKT | GCATGTTGATTTTCGTAAAGACGTTGACTGGA |
| | | IELEVEPSDTIENV | AAGACTATCGAGTTGGAAGTGGAGCCTTCCG |
| | | KAKIQDKEGIPPD | ATACTATCGAGAATGTTAAGGCCAAAATCCA |
| | | QQRLAFAGKSLE | AGATAAGGAAGGGATTCCTCCAGATCAACAA |
| | | DGRTLSDYNILKD | CGCCTTGCTTTTGCCGGGAAGAGCCTGGAGG |
| | | SKLHPLLRLR | ACGGTCGCACACTGTCTGACTATAACATTCTT |
| | | | AAAGATTCTAAATTGCATCCACTGCTGCGCTT |
| | | | GCGT |
| i53 K33A | K33A | MHHHHHHGGS | ATGCACCATCACCACCACCACGGGGGGTCGG |
| [237; 887] | | GMLIFVKTLTGKT | GCATGTTGATTTTCGTAAAGACGTTGACTGGA |
| | | ITLEVEPSDTIENV | AAGACTATCACTTTGGAAGTGGAGCCTTCCG |
| | | KAKIQDAEGIPPD | ATACTATCGAGAATGTTAAGGCCAAAATCCA |
| | | QQRLAFAGKSLE | AGATGCCGAAGGGATTCCTCCAGATCAACAA |
| | | DGRTLSDYNILKD | CGCCTTGCTTTTGCCGGGAAGAGCCTGGAGG |
| | | SKLHPLLRLR | ACGGTCGCACACTGTCTGACTATAACATTCTT |
| | | | AAAGATTCTAAATTGCATCCACTGCTGCGCTT |
| | | | GCGT |
| i53 A46Q | A46Q | MHHHHHHGGS | ATGCACCATCACCACCACCACGGGGGGTCGG |
| [238; 888] | | GMLIFVKTLTGKT | GCATGTTGATTTTCGTAAAGACGTTGACTGGA |
| | | ITLEVEPSDTIENV | AAGACTATCACTTTGGAAGTGGAGCCTTCCG |
| | | KAKIQDKEGIPPD | ATACTATCGAGAATGTTAAGGCCAAAATCCA |
| | | QQRLAFQGKSLE | AGATAAGGAAGGGATTCCTCCAGATCAACAA |
| | | DGRTLSDYNILKD | CGCCTTGCTTTTCAAGGGAAGAGCCTGGAGG |
| | | SKLHPLLRLR | ACGGTCGCACACTGTCTGACTATAACATTCTT |
| | | | AAAGATTCTAAATTGCATCCACTGCTGCGCTT |
| | | | GCGT |
| i53 K63I | K63I | MHHHHHHGGS | ATGCACCATCACCACCACCACGGGGGGTCGG |
| [239; 889] | | GMLIFVKTLTGKT | GCATGTTGATTTTCGTAAAGACGTTGACTGGA |
| | | ITLEVEPSDTIENV | AAGACTATCACTTTGGAAGTGGAGCCTTCCG |
| | | KAKIQDKEGIPPD | ATACTATCGAGAATGTTAAGGCCAAAATCCA |
| | | QQRLAFAGKSLE | AGATAAGGAAGGGATTCCTCCAGATCAACAA |
| | | DGRTLSDYNILID | CGCCTTGCTTTTGCCGGGAAGAGCCTGGAGG |
| | | SKLHPLLRLR | ACGGTCGCACACTGTCTGACTATAACATTCTT |
| | | | ATTGATTCTAAATTGCATCCACTGCTGCGCTT |
| | | | GCGT |
| i53 S65P | S65P | MHHHHHHGGS | ATGCACCATCACCACCACCACGGGGGGTCGG |
| [240; 890] | | GMLIFVKTLTGKT | GCATGTTGATTTTCGTAAAGACGTTGACTGGA |
| | | ITLEVEPSDTIENV | AAGACTATCACTTTGGAAGTGGAGCCTTCCG |
| | | KAKIQDKEGIPPD | ATACTATCGAGAATGTTAAGGCCAAAATCCA |
| | | QQRLAFAGKSLE | AGATAAGGAAGGGATTCCTCCAGATCAACAA |
| | | DGRTLSDYNILKD | CGCCTTGCTTTTGCCGGGAAGAGCCTGGAGG |
| | | PKLHPLLRLR | ACGGTCGCACACTGTCTGACTATAACATTCTT |
| | | | AAAGATCCTAAATTGCATCCACTGCTGCGCTT |
| | | | GCGT |
| MBP | N/A | MKIEEGKLVIWIN | ATGAAAATCGAAGAAGGTAAACTGGTAATCT |
| tagged | | GDKGYNGLAEV | GGATTAACGGCGATAAAGGCTATAACGGTCT |
| 53BP1 | | GKKFEKDTGIKVT | CGCTGAAGTCGGTAAGAAATTCGAGAAAGAT |
| fragment | | VEHPDKLEEKFP | ACCGGAATTAAAGTCACCGTTGAGCATCCGG |
| [245; 891] | | QVAATGDGPDII | ATAAACTGGAAGAGAAATTCCCACAGGTTGC |
| | | FWAHDRFGGYA | GGCAACTGGCGATGGCCCTGACATTATCTTCT |
| | | QSGLLAEITPDKA | GGGCACACGACCGCTTTGGTGGCTACGCTCA |
| | | FQDKLYPFTWDA | ATCTGGCCTGTTGGCTGAAATCACCCCGGAC |
| | | VRYNGKLIAYPIA | AAAGCGTTCCAGGACAAGCTGTATCCGTTTAC |
| | | VEALSLIYNKDLL | CTGGGATGCCGTACGTTACAACGGCAAGCTG |
| | | PNPPKTWEEIPA | ATTGCTTACCCGATCGCTGTTGAAGCGTTATC |
| | | LDKELKAKGKSAL | GCTGATTTATAACAAAGATCTGCTGCCGAACC |
| | | MFNLQEPYFTW | CGCCAAAAACCTGGGAAGAGATCCCGGCGCT |
| | | PLIAADGGYAFKY | GGATAAAGAACTGAAAGCGAAAGGTAAGAG |
| | | ENGKYDIKDVGV | CGCGCTGATGTTCAACCTGCAAGAACCGTACT |
| | | DNAGAKAGLTFL | TCACCTGGCCGCTGATTGCTGCTGACGGGGG |
| | | VDLIKNKHMNA | TTATGCGTTCAAGTATGAAAACGGCAAGTAC |
| | | DTDYSIAEAAFNK | GACATTAAAGACGTGGGCGTGGATAACGCTG |
| | | GETAMTINGPW | GCGCGAAAGCGGGTCTGACCTTCCTGGTTGA |
| | | AWSNIDTSKVNY | CCTGATTAAAAACAAACACATGAATGCAGAC |
| | | GVTVLPTFKGQP | ACCGATTACTCCATCGCAGAAGCTGCCTTTAA |
| | | SKPFVGVLSAGIN | TAAAGGCGAAACAGCGATGACCATCAACGGC |
| | | AASPNKELAKEFL | CCGTGGGCATGGTCCAACATCGACACCAGCA |
| | | ENYLLTDEGLEAV | AAGTGAATTATGGTGTAACGGTACTGCCGAC |
| | | NKDKPLGAVALK | CTTCAAGGGTCAACCATCCAAACCGTTCGTTG |
| | | SYEEELAKDPRIA | GCGTGCTGAGCGCAGGTATTAACGCCGCCAG |
| | | ATMENAQKGEI | TCCGAACAAAGAGCTGGCAAAAGAGTTCCTC |
| | | MPNIPQMSAFW | GAAAACTATCTGCTGACTGATGAAGGTCTGG |
| | | YAVRTAVINAAS | AAGCGGTTAATAAAGACAAACCGCTGGGTGC |
| | | GRQTVDEALKDA | CGTAGCGCTGAAGTCTTACGAGGAAGAGTTG |
| | | QTNSSSNNNNN | GCGAAAGATCCACGTATTGCCGCCACTATGG |
| | | NNNNNLGIEENL | AAAACGCCCAGAAAGGTGAAATCATGCCGAA |
| | | YFQGHMNSFVG | CATCCCGCAGATGTCCGCTTTCTGGTATGCCG |
| | | LRVVAKWSSNGY | TGCGTACTGCGGTGATCAACGCCGCCAGCGG |
| | | FYSGKITRDVGA | TCGTCAGACTGTCGATGAAGCCCTGAAAGAC |
| | | GKYKLLFDDGYE | GCGCAGACTAATTCGAGCTCGAACAACAACA |
| | | CDVLGKDILLCDP | ACAATAACAATAACAACAACCTCGGGATCGA |
| | | IPLDTEVTALSED | GGAAAATCTGTATTTTCAGGGCCACATGAAT |
| | | EYFSAGVVKGHR | AGCTTTGTTGGTCTGCGTGTTGTTGCAAAATG |
| | | KESGELYYSIEKE | GTCAAGCAATGGTTATTTCTACAGCGGCAAA |
| | | GQRKWYKRMA | ATCACCCGTGATGTTGGTGCAGGTAAATACA |
| | | VILSLEQGNRLRE | AACTGCTGTTTGATGATGGTTATGAATGTGAT |
| | | QYGLG | GTGCTGGGCAAAGATATTCTGCTGTGTGATC |
| | | | CGATTCCGCTGGATACCGAAGTTACCGCACT |
| | | | GAGCGAAGATGAATATTTCAGTGCCGGTGTT |
| | | | GTTAAAGGCCATCGTAAAGAAAGCGGTGAAC |
| | | | TGTATTACAGCATTGAAAAAGAAGGTCAGCG |
| | | | CAAATGGTATAAACGTATGGCAGTTATTCTGA |
| | | | GCCTGGAACAGGGTAATCGTCTGCGTGAACA |
| | | | GTATGGTCTGGGT |
| SM1 | M1H | MHHHHHHGGS | ATGCACCACCACCACCACCACGGTGGATCTG |
| [246; 892] | | GHLIFVKTLTGKTI | GCCATTTGATTTTCGTAAAGACGTTGACTGGA |
| | | TLEVEPSDTIENV | AAGACTATCACTTTGGAAGTGGAGCCTTCCG |
| | | KAKIQDKEGIPPD | ATACTATCGAGAATGTTAAGGCCAAAATCCA |
| | | QQRLAFAGKSLE | AGATAAGGAAGGGATTCCTCCAGATCAACAA |
| | | DGRTLSDYNILKD | CGCCTTGCTTTTGCCGGGAAGAGCCTGGAGG |
| | | SKLHPLLRLR | ACGGTCGCACACTGTCTGACTATAACATTCTT |
| | | | AAAGATTCTAAATTGCATCCACTGCTGCGCTT |
| | | | GCGT |
| SM2 | K6R | MHHHHHHGGS | ATGCACCACCACCACCACCACGGTGGATCTG |
| [247; 893] | | GMLIFVRTLTGKT | GCATGTTGATTTTCGTACGCACGTTGACTGGA |
| | | ITLEVEPSDTIENV | AAGACTATCACTTTGGAAGTGGAGCCTTCCG |
| | | KAKIQDKEGIPPD | ATACTATCGAGAATGTTAAGGCCAAAATCCA |
| | | QQRLAFAGKSLE | AGATAAGGAAGGGATTCCTCCAGATCAACAA |
| | | DGRTLSDYNILKD | CGCCTTGCTTTTGCCGGGAAGAGCCTGGAGG |
| | | SKLHPLLRLR | ACGGTCGCACACTGTCTGACTATAACATTCTT |
| | | | AAAGATTCTAAATTGCATCCACTGCTGCGCTT |
| | | | GCGT |
| SM3 | T7M | MHHHHHHGGS | ATGCACCACCACCACCACCACGGTGGATCTG |
| [248; 894] | | GMLIFVKMLTGK | GCATGTTGATTTTCGTAAAGATGTTGACTGGA |
| | | TITLEVEPSDTIEN | AAGACTATCACTTTGGAAGTGGAGCCTTCCG |
| | | VKAKIQDKEGIPP | ATACTATCGAGAATGTTAAGGCCAAAATCCA |
| | | DQQRLAFAGKSL | AGATAAGGAAGGGATTCCTCCAGATCAACAA |
| | | EDGRTLSDYNILK | CGCCTTGCTTTTGCCGGGAAGAGCCTGGAGG |
| | | DSKLHPLLRLR | ACGGTCGCACACTGTCTGACTATAACATTCTT |
| | | | AAAGATTCTAAATTGCATCCACTGCTGCGCTT |
| | | | GCGT |
| SM4 | T12M | MHHHHHHGGS | ATGCACCACCACCACCACCACGGTGGATCTG |
| [249; 895] | | GMLIFVKTLTGK | GCATGTTGATTTTCGTAAAGACGTTGACTGGA |
| | | MITLEVEPSDTIE | AAGATGATCACTTTGGAAGTGGAGCCTTCCG |
| | | NVKAKIQDKEGIP | ATACTATCGAGAATGTTAAGGCCAAAATCCA |
| | | PDQQRLAFAGKS | AGATAAGGAAGGGATTCCTCCAGATCAACAA |
| | | LEDGRTLSDYNIL | CGCCTTGCTTTTGCCGGGAAGAGCCTGGAGG |
| | | KDSKLHPLLRLR | ACGGTCGCACACTGTCTGACTATAACATTCTT |
| | | | AAAGATTCTAAATTGCATCCACTGCTGCGCTT |
| | | | GCGT |
| SM5 | T14E | MHHHHHHGGS | ATGCACCACCACCACCACCACGGTGGATCTG |
| [250; 896] | | GMLIFVKTLTGKT | GCATGTTGATTTTCGTAAAGACGTTGACTGGA |
| | | IELEVEPSDTIENV | AAGACTATCGAGTTGGAAGTGGAGCCTTCCG |
| | | KAKIQDKEGIPPD | ATACTATCGAGAATGTTAAGGCCAAAATCCA |
| | | QQRLAFAGKSLE | AGATAAGGAAGGGATTCCTCCAGATCAACAA |
| | | DGRTLSDYNILKD | CGCCTTGCTTTTGCCGGGAAGAGCCTGGAGG |
| | | SKLHPLLRLR | ACGGTCGCACACTGTCTGACTATAACATTCTT |
| | | | AAAGATTCTAAATTGCATCCACTGCTGCGCTT |
| | | | GCGT |
| SM6 | E16M | MHHHHHHGGS | ATGCACCACCACCACCACCACGGTGGATCTG |
| [251; 897] | | GMLIFVKTLTGKT | GCATGTTGATTTTCGTAAAGACGTTGACTGGA |
| | | ITLMVEPSDTIEN | AAGACTATCACTTTGATGGTGGAGCCTTCCGA |
| | | VKAKIQDKEGIPP | TACTATCGAGAATGTTAAGGCCAAAATCCAA |
| | | DQQRLAFAGKSL | GATAAGGAAGGGATTCCTCCAGATCAACAAC |
| | | EDGRTLSDYNILK | GCCTTGCTTTTGCCGGGAAGAGCCTGGAGGA |
| | | DSKLHPLLRLR | CGGTCGCACACTGTCTGACTATAACATTCTTA |
| | | | AAGATTCTAAATTGCATCCACTGCTGCGCTTG |
| | | | CGT |
| SM7 | E18M | MHHHHHHGGS | ATGCACCACCACCACCACCACGGTGGATCTG |
| [252; 898] | | GMLIFVKTLTGKT | GCATGTTGATTTTCGTAAAGACGTTGACTGGA |
| | | ITLEVMPSDTIEN | AAGACTATCACTTTGGAAGTGATGCCTTCCGA |
| | | VKAKIQDKEGIPP | TACTATCGAGAATGTTAAGGCCAAAATCCAA |
| | | DQQRLAFAGKSL | GATAAGGAAGGGATTCCTCCAGATCAACAAC |
| | | EDGRTLSDYNILK | GCCTTGCTTTTGCCGGGAAGAGCCTGGAGGA |
| | | DSKLHPLLRLR | CGGTCGCACACTGTCTGACTATAACATTCTTA |
| | | | AAGATTCTAAATTGCATCCACTGCTGCGCTTG |
| | | | CGT |
| SM8 | N25V | MHHHHHHGGS | ATGCACCACCACCACCACCACGGTGGATCTG |
| [253; 899] | | GMLIFVKTLTGKT | GCATGTTGATTTTCGTAAAGACGTTGACTGGA |
| | | ITLEVEPSDTIEVV | AAGACTATCACTTTGGAAGTGGAGCCTTCCG |
| | | KAKIQDKEGIPPD | ATACTATCGAGGTAGTTAAGGCCAAAATCCA |
| | | QQRLAFAGKSLE | AGATAAGGAAGGGATTCCTCCAGATCAACAA |
| | | DGRTLSDYNILKD | CGCCTTGCTTTTGCCGGGAAGAGCCTGGAGG |
| | | SKLHPLLRLR | ACGGTCGCACACTGTCTGACTATAACATTCTT |
| | | | AAAGATTCTAAATTGCATCCACTGCTGCGCTT |
| | | | GCGT |
| SM9 | V26I | MHHHHHHGGS | ATGCACCACCACCACCACCACGGTGGATCTG |
| [254; 900] | | GMLIFVKTLTGKT | GCATGTTGATTTTCGTAAAGACGTTGACTGGA |
| | | ITLEVEPSDTIENI | AAGACTATCACTTTGGAAGTGGAGCCTTCCG |
| | | KAKIQDKEGIPPD | ATACTATCGAGAATATTAAGGCCAAAATCCAA |
| | | QQRLAFAGKSLE | GATAAGGAAGGGATTCCTCCAGATCAACAAC |
| | | DGRTLSDYNILKD | GCCTTGCTTTTGCCGGGAAGAGCCTGGAGGA |
| | | SKLHPLLRLR | CGGTCGCACACTGTCTGACTATAACATTCTTA |
| | | | AAGATTCTAAATTGCATCCACTGCTGCGCTTG |
| | | | CGT |
| SM10 | Q31W | MHHHHHHGGS | ATGCACCACCACCACCACCACGGTGGATCTG |
| [255; 901] | | GMLIFVKTLTGKT | GCATGTTGATTTTCGTAAAGACGTTGACTGGA |
| | | ITLEVEPSDTIENV | AAGACTATCACTTTGGAAGTGGAGCCTTCCG |
| | | KAKIWDKEGIPP | ATACTATCGAGAATGTTAAGGCCAAAATCTG |
| | | DQQRLAFAGKSL | GGATAAGGAAGGGATTCCTCCAGATCAACAA |
| | | EDGRTLSDYNILK | CGCCTTGCTTTTGCCGGGAAGAGCCTGGAGG |
| | | DSKLHPLLRLR | ACGGTCGCACACTGTCTGACTATAACATTCTT |
| | | | AAAGATTCTAAATTGCATCCACTGCTGCGCTT |
| | | | GCGT |
| SM11 | Q31C | MHHHHHHGGS | ATGCACCACCACCACCACCACGGTGGATCTG |
| [256; 902] | | GMLIFVKTLTGKT | GCATGTTGATTTTCGTAAAGACGTTGACTGGA |
| | | ITLEVEPSDTIENV | AAGACTATCACTTTGGAAGTGGAGCCTTCCG |
| | | KAKICDKEGIPPD | ATACTATCGAGAATGTTAAGGCCAAAATCTG |
| | | QQRLAFAGKSLE | CGATAAGGAAGGGATTCCTCCAGATCAACAA |
| | | DGRTLSDYNILKD | CGCCTTGCTTTTGCCGGGAAGAGCCTGGAGG |
| | | SKLHPLLRLR | ACGGTCGCACACTGTCTGACTATAACATTCTT |
| | | | AAAGATTCTAAATTGCATCCACTGCTGCGCTT |
| | | | GCGT |
| SM12 | Q31F | MHHHHHHGGS | ATGCACCACCACCACCACCACGGTGGATCTG |
| [257; 903] | | GMLIFVKTLTGKT | GCATGTTGATTTTCGTAAAGACGTTGACTGGA |
| | | ITLEVEPSDTIENV | AAGACTATCACTTTGGAAGTGGAGCCTTCCG |
| | | KAKIFDKEGIPPD | ATACTATCGAGAATGTTAAGGCCAAAATCTTC |
| | | QQRLAFAGKSLE | GATAAGGAAGGGATTCCTCCAGATCAACAAC |
| | | DGRTLSDYNILKD | GCCTTGCTTTTGCCGGGAAGAGCCTGGAGGA |
| | | SKLHPLLRLR | CGGTCGCACACTGTCTGACTATAACATTCTTA |
| | | | AAGATTCTAAATTGCATCCACTGCTGCGCTTG |
| | | | CGT |
| SM13 | K33S | MHHHHHHGGS | ATGCACCACCACCACCACCACGGTGGATCTG |
| [258; 904] | | GMLIFVKTLTGKT | GCATGTTGATTTTCGTAAAGACGTTGACTGGA |
| | | ITLEVEPSDTIENV | AAGACTATCACTTTGGAAGTGGAGCCTTCCG |
| | | KAKIQDSEGIPPD | ATACTATCGAGAATGTTAAGGCCAAAATCCA |
| | | QQRLAFAGKSLE | AGATTCTGAAGGGATTCCTCCAGATCAACAA |
| | | DGRTLSDYNILKD | CGCCTTGCTTTTGCCGGGAAGAGCCTGGAGG |
| | | SKLHPLLRLR | ACGGTCGCACACTGTCTGACTATAACATTCTT |
| | | | AAAGATTCTAAATTGCATCCACTGCTGCGCTT |
| | | | GCGT |
| SM14 | K33H | MHHHHHHGGS | ATGCACCACCACCACCACCACGGTGGATCTG |
| [259; 905] | | GMLIFVKTLTGKT | GCATGTTGATTTTCGTAAAGACGTTGACTGGA |
| | | ITLEVEPSDTIENV | AAGACTATCACTTTGGAAGTGGAGCCTTCCG |
| | | KAKIQDHEGIPP | ATACTATCGAGAATGTTAAGGCCAAAATCCA |
| | | DQQRLAFAGKSL | AGATCATGAAGGGATTCCTCCAGATCAACAA |
| | | EDGRTLSDYNILK | CGCCTTGCTTTTGCCGGGAAGAGCCTGGAGG |
| | | DSKLHPLLRLR | ACGGTCGCACACTGTCTGACTATAACATTCTT |
| | | | AAAGATTCTAAATTGCATCCACTGCTGCGCTT |
| | | | GCGT |
| SM15 | K33A | MHHHHHHGGS | ATGCACCACCACCACCACCACGGTGGATCTG |
| [260; 906] | | GMLIFVKTLTGKT | GCATGTTGATTTTCGTAAAGACGTTGACTGGA |
| | | ITLEVEPSDTIENV | AAGACTATCACTTTGGAAGTGGAGCCTTCCG |
| | | KAKIQDAEGIPPD | ATACTATCGAGAATGTTAAGGCCAAAATCCA |
| | | QQRLAFAGKSLE | AGATGCCGAAGGGATTCCTCCAGATCAACAA |
| | | DGRTLSDYNILKD | CGCCTTGCTTTTGCCGGGAAGAGCCTGGAGG |
| | | SKLHPLLRLR | ACGGTCGCACACTGTCTGACTATAACATTCTT |
| | | | AAAGATTCTAAATTGCATCCACTGCTGCGCTT |
| | | | GCGT |
| SM16 | P38L | MHHHHHHGGS | ATGCACCACCACCACCACCACGGTGGATCTG |
| [261; 907] | | GMLIFVKTLTGKT | GCATGTTGATTTTCGTAAAGACGTTGACTGGA |
| | | ITLEVEPSDTIENV | AAGACTATCACTTTGGAAGTGGAGCCTTCCG |
| | | KAKIQDKEGIPLD | ATACTATCGAGAATGTTAAGGCCAAAATCCA |
| | | QQRLAFAGKSLE | AGATAAGGAAGGGATTCCTTTGGATCAACAA |
| | | DGRTLSDYNILKD | CGCCTTGCTTTTGCCGGGAAGAGCCTGGAGG |
| | | SKLHPLLRLR | ACGGTCGCACACTGTCTGACTATAACATTCTT |
| | | | AAAGATTCTAAATTGCATCCACTGCTGCGCTT |
| | | | GCGT |
| SM17 | R42W | MHHHHHHGGS | ATGCACCACCACCACCACCACGGTGGATCTG |
| [262; 908] | | GMLIFVKTLTGKT | GCATGTTGATTTTCGTAAAGACGTTGACTGGA |
| | | ITLEVEPSDTIENV | AAGACTATCACTTTGGAAGTGGAGCCTTCCG |
| | | KAKIQDKEGIPPD | ATACTATCGAGAATGTTAAGGCCAAAATCCA |
| | | QQWLAFAGKSL | AGATAAGGAAGGGATTCCTCCAGATCAACAA |
| | | EDGRTLSDYNILK | TGGCTTGCTTTTGCCGGGAAGAGCCTGGAGG |
| | | DSKLHPLLRLR | ACGGTCGCACACTGTCTGACTATAACATTCTT |
| | | | AAAGATTCTAAATTGCATCCACTGCTGCGCTT |
| | | | GCGT |
| SM18 | A44T | MHHHHHHGGS | ATGCACCACCACCACCACCACGGTGGATCTG |
| [263; 909] | | GMLIFVKTLTGKT | GCATGTTGATTTTCGTAAAGACGTTGACTGGA |
| | | ITLEVEPSDTIENV | AAGACTATCACTTTGGAAGTGGAGCCTTCCG |
| | | KAKIQDKEGIPPD | ATACTATCGAGAATGTTAAGGCCAAAATCCA |
| | | QQRLTFAGKSLE | AGATAAGGAAGGGATTCCTCCAGATCAACAA |
| | | DGRTLSDYNILKD | CGCCTTACTTTTGCCGGGAAGAGCCTGGAGG |
| | | SKLHPLLRLR | ACGGTCGCACACTGTCTGACTATAACATTCTT |
| | | | AAAGATTCTAAATTGCATCCACTGCTGCGCTT |
| | | | GCGT |
| SM19 | A46Q | MHHHHHHGGS | ATGCACCACCACCACCACCACGGTGGATCTG |
| [264; 910] | | GMLIFVKTLTGKT | GCATGTTGATTTTCGTAAAGACGTTGACTGGA |
| | | ITLEVEPSDTIENV | AAGACTATCACTTTGGAAGTGGAGCCTTCCG |
| | | KAKIQDKEGIPPD | ATACTATCGAGAATGTTAAGGCCAAAATCCA |
| | | QQRLAFQGKSLE | AGATAAGGAAGGGATTCCTCCAGATCAACAA |
| | | DGRTLSDYNILKD | CGCCTTGCTTTTCAAGGGAAGAGCCTGGAGG |
| | | SKLHPLLRLR | ACGGTCGCACACTGTCTGACTATAACATTCTT |
| | | | AAAGATTCTAAATTGCATCCACTGCTGCGCTT |
| | | | GCGT |
| SM20 | K48T | MHHHHHHGGS | ATGCACCACCACCACCACCACGGTGGATCTG |
| [265; 911] | | GMLIFVKTLTGKT | GCATGTTGATTTTCGTAAAGACGTTGACTGGA |
| | | ITLEVEPSDTIENV | AAGACTATCACTTTGGAAGTGGAGCCTTCCG |
| | | KAKIQDKEGIPPD | ATACTATCGAGAATGTTAAGGCCAAAATCCA |
| | | QQRLAFAGTSLE | AGATAAGGAAGGGATTCCTCCAGATCAACAA |
| | | DGRTLSDYNILKD | CGCCTTGCTTTTGCCGGGACTAGCCTGGAGG |
| | | SKLHPLLRLR | ACGGTCGCACACTGTCTGACTATAACATTCTT |
| | | | AAAGATTCTAAATTGCATCCACTGCTGCGCTT |
| | | | GCGT |
| SM21 | R54Y | MHHHHHHGGS | ATGCACCACCACCACCACCACGGTGGATCTG |
| [266; 912] | | GMLIFVKTLTGKT | GCATGTTGATTTTCGTAAAGACGTTGACTGGA |
| | | ITLEVEPSDTIENV | AAGACTATCACTTTGGAAGTGGAGCCTTCCG |
| | | KAKIQDKEGIPPD | ATACTATCGAGAATGTTAAGGCCAAAATCCA |
| | | QQRLAFAGKSLE | AGATAAGGAAGGGATTCCTCCAGATCAACAA |
| | | DGYTLSDYNILKD | CGCCTTGCTTTTGCCGGGAAGAGCCTGGAGG |
| | | SKLHPLLRLR | ACGGTTATACACTGTCTGACTATAACATTCTT |
| | | | AAAGATTCTAAATTGCATCCACTGCTGCGCTT |
| | | | GCGT |
| SM22 | S57G | MHHHHHHGGS | ATGCACCACCACCACCACCACGGTGGATCTG |
| [267; 913] | | GMLIFVKTLTGKT | GCATGTTGATTTTCGTAAAGACGTTGACTGGA |
| | | ITLEVEPSDTIENV | AAGACTATCACTTTGGAAGTGGAGCCTTCCG |
| | | KAKIQDKEGIPPD | ATACTATCGAGAATGTTAAGGCCAAAATCCA |
| | | QQRLAFAGKSLE | AGATAAGGAAGGGATTCCTCCAGATCAACAA |
| | | DGRTLGDYNILK | CGCCTTGCTTTTGCCGGGAAGAGCCTGGAGG |
| | | DSKLHPLLRLR | ACGGTCGCACACTGGGGGACTATAACATTCT |
| | | | TAAAGATTCTAAATTGCATCCACTGCTGCGCT |
| | | | TGCGT |
| SM23 | K63I | MHHHHHHGGS | ATGCACCACCACCACCACCACGGTGGATCTG |
| [268; 914] | | GMLIFVKTLTGKT | GCATGTTGATTTTCGTAAAGACGTTGACTGGA |
| | | ITLEVEPSDTIENV | AAGACTATCACTTTGGAAGTGGAGCCTTCCG |
| | | KAKIQDKEGIPPD | ATACTATCGAGAATGTTAAGGCCAAAATCCA |
| | | QQRLAFAGKSLE | AGATAAGGAAGGGATTCCTCCAGATCAACAA |
| | | DGRTLSDYNILID | CGCCTTGCTTTTGCCGGGAAGAGCCTGGAGG |
| | | SKLHPLLRLR | ACGGTCGCACACTGTCTGACTATAACATTCTT |
| | | | ATTGATTCTAAATTGCATCCACTGCTGCGCTT |
| | | | GCGT |
| SM24 | S65P | MHHHHHHGGS | ATGCACCACCACCACCACCACGGTGGATCTG |
| [269; 915] | | GMLIFVKTLTGKT | GCATGTTGATTTTCGTAAAGACGTTGACTGGA |
| | | ITLEVEPSDTIENV | AAGACTATCACTTTGGAAGTGGAGCCTTCCG |
| | | KAKIQDKEGIPPD | ATACTATCGAGAATGTTAAGGCCAAAATCCA |
| | | QQRLAFAGKSLE | AGATAAGGAAGGGATTCCTCCAGATCAACAA |
| | | DGRTLSDYNILKD | CGCCTTGCTTTTGCCGGGAAGAGCCTGGAGG |
| | | PKLHPLLRLR | ACGGTCGCACACTGTCTGACTATAACATTCTT |
| | | | AAAGATCCTAAATTGCATCCACTGCTGCGCTT |
| | | | GCGT |
| CM1 | K6R, T7M, | MHHHHHHGGS | ATGCACCACCACCACCACCACGGTGGATCTG |
| [241; 916] | T12M, T14E, | GMLIFVRMLTGK | GCATGTTGATTTTCGTACGCATGTTGACTGGA |
| | K33H, A46Q, | MIELEVEPSDTIE | AAGATGATCGAGTTGGAAGTGGAGCCTTCCG |
| | S65P, L67K, | NVKAKIQDHEGI | ATACTATCGAGAATGTTAAGGCCAAAATCCA |
| | H68M | PPDQQRLAFQG | AGATCATGAAGGGATTCCTCCAGATCAACAA |
| | | KSLEDGRTLSDY | CGCCTTGCTTTTCAAGGGAAGAGCCTGGAGG |
| | | NILKDPKKMPLLR | ACGGTCGCACACTGTCTGACTATAACATTCTT |
| | | LR | AAAGATCCTAAAAAGATGCCACTGCTGCGCT |
| | | | TGCGT |
| CM7 | K6R, K33H, | MHHHHHHGGS | ATGCACCACCACCACCACCACGGTGGATCTG |
| [242; 917] | A46Q, S65P | GMLIFVRTLTGKT | GCATGTTGATTTTCGTACGCACGTTGACTGGA |
| | | ITLEVEPSDTIENV | AAGACTATCACTTTGGAAGTGGAGCCTTCCG |
| | | KAKIQDHEGIPP | ATACTATCGAGAATGTTAAGGCCAAAATCCA |
| | | DQQRLAFQGKSL | AGATCATGAAGGGATTCCTCCAGATCAACAA |
| | | EDGRTLSDYNILK | CGCCTTGCTTTTCAAGGGAAGAGCCTGGAGG |
| | | DPKLHPLLRLR | ACGGTCGCACACTGTCTGACTATAACATTCTT |
| | | | AAAGATCCTAAATTGCATCCACTGCTGCGCTT |
| | | | GCGT |
| CM13 | T7M, T14E, | MHHHHHHGGS | ATGCACCATCACCACCACCACGGTGGATCTG |
| [243; 918] | A46Q, L67K | GMLIFVKMLTGK | GCATGTTGATTTTCGTAAAGATGTTGACTGGA |
| | | TIELEVEPSDTIEN | AAGACTATCGAGTTGGAAGTGGAGCCTTCCG |
| | | VKAKIQDKEGIPP | ATACTATCGAGAATGTTAAGGCCAAAATCCA |
| | | DQQRLAFQGKSL | AGATAAGGAAGGGATTCCTCCAGATCAACAA |
| | | EDGRTLSDYNILK | CGCCTTGCTTTTCAAGGGAAGAGCCTGGAGG |
| | | DSKKHPLLRLR | ACGGTCGCACACTGTCTGACTATAACATTCTT |
| | | | AAAGATTCTAAAAAGCATCCACTGCTGCGCTT |
| | | | GCGT |
| CM26 | T12M, K33H, | MHHHHHHGGS | ATGCACCATCACCACCACCACGGTGGATCTG |
| [244; 919] | A46Q, H68M | GMLIFVKTLTGK | GCATGTTGATTTTCGTAAAGACGTTGACTGGA |
| | | MITLEVEPSDTIE | AAGATGATCACTTTGGAAGTGGAGCCTTCCG |
| | | NVKAKIQDHEGI | ATACTATCGAGAATGTTAAGGCCAAAATCCA |
| | | PPDQQRLAFQG | AGATCATGAAGGGATTCCTCCAGATCAACAA |
| | | KSLEDGRTLSDY | CGCCTTGCTTTTCAAGGGAAGAGCCTGGAGG |
| | | NILKDSKLMPLLR | ACGGTCGCACACTGTCTGACTATAACATTCTT |
| | | LR | AAAGATTCTAAATTGATGCCACTGCTGCGCTT |
| | | | GCGT |
| CM44 | T7M, T12M, | MHHHHHHGGS | ATGCACCACCACCACCACCACGGTGGATCTG |
| [270; 920] | T14E, K33H, | GMLIFVKMLTGK | GCATGTTGATTTTCGTAAAGATGTTGACTGGA |
| | A46Q, S65P, | MIELEVEPSDTIE | AAGATGATCGAGTTGGAAGTGGAGCCTTCCG |
| | L67K, H68M | NVKAKIQDHEGI | ATACTATCGAGAATGTTAAGGCCAAAATCCA |
| | | PPDQQRLAFQG | AGATCATGAAGGGATTCCTCCAGATCAACAA |
| | | KSLEDGRTLSDY | CGCCTTGCTTTTCAAGGGAAGAGCCTGGAGG |
| | | NILKDPKKMPLLR | ACGGTCGCACACTGTCTGACTATAACATTCTT |
| | | LR | AAAGATCCTAAAAAGATGCCACTGCTGCGCT |
| | | | TGCGT |
| CM45 | K6R, T12M, | MHHHHHHGGS | ATGCACCACCACCACCACCACGGTGGATCTG |
| [271; 921] | T14E, K33H, | GMLIFVRTLTGK | GCATGTTGATTTTCGTACGCACGTTGACTGGA |
| | A46Q, S65P, | MIELEVEPSDTIE | AAGATGATCGAGTTGGAAGTGGAGCCTTCCG |
| | L67K, H68M | NVKAKIQDHEGI | ATACTATCGAGAATGTTAAGGCCAAAATCCA |
| | | PPDQQRLAFQG | AGATCATGAAGGGATTCCTCCAGATCAACAA |
| | | KSLEDGRTLSDY | CGCCTTGCTTTTCAAGGGAAGAGCCTGGAGG |
| | | NILKDPKKMPLLR | ACGGTCGCACACTGTCTGACTATAACATTCTT |
| | | LR | AAAGATCCTAAAAAGATGCCACTGCTGCGCT |
| | | | TGCGT |
| CM46 | K6R, T7M, | MHHHHHHGGS | ATGCACCACCACCACCACCACGGTGGATCTG |
| [272; 922] | T14E, K33H, | GMLIFVRMLTGK | GCATGTTGATTTTCGTACGCATGTTGACTGGA |
| | A46Q, S65P, | TIELEVEPSDTIEN | AAGACTATCGAGTTGGAAGTGGAGCCTTCCG |
| | L67K, H68M | VKAKIQDHEGIPP | ATACTATCGAGAATGTTAAGGCCAAAATCCA |
| | | DQQRLAFQGKSL | AGATCATGAAGGGATTCCTCCAGATCAACAA |
| | | EDGRTLSDYNILK | CGCCTTGCTTTTCAAGGGAAGAGCCTGGAGG |
| | | DPKKMPLLRLR | ACGGTCGCACACTGTCTGACTATAACATTCTT |
| | | | AAAGATCCTAAAAAGATGCCACTGCTGCGCT |
| | | | TGCGT |
| CM47 | K6R, T7M, | MHHHHHHGGS | ATGCACCACCACCACCACCACGGTGGATCTG |
| [273; 923] | T12M, K33H, | GMLIFVRMLTGK | GCATGTTGATTTTCGTACGCATGTTGACTGGA |
| | A46Q, S65P, | MITLEVEPSDTIE | AAGATGATCACTTTGGAAGTGGAGCCTTCCG |
| | L67K, H68M | NVKAKIQDHEGI | ATACTATCGAGAATGTTAAGGCCAAAATCCA |
| | | PPDQQRLAFQG | AGATCATGAAGGGATTCCTCCAGATCAACAA |
| | | KSLEDGRTLSDY | CGCCTTGCTTTTCAAGGGAAGAGCCTGGAGG |
| | | NILKDPKKMPLLR | ACGGTCGCACACTGTCTGACTATAACATTCTT |
| | | LR | AAAGATCCTAAAAAGATGCCACTGCTGCGCT |
| | | | TGCGT |
| CM48 | K6R, T7M, | MHHHHHHGGS | ATGCACCACCACCACCACCACGGTGGATCTG |
| [274; 924] | T12M, T14E, | GMLIFVRMLTGK | GCATGTTGATTTTCGTACGCATGTTGACTGGA |
| | A46Q, S65P, | MIELEVEPSDTIE | AAGATGATCGAGTTGGAAGTGGAGCCTTCCG |
| | L67K, H68M | NVKAKIQDKEGIP | ATACTATCGAGAATGTTAAGGCCAAAATCCA |
| | | PDQQRLAFQGKS | AGATAAGGAAGGGATTCCTCCAGATCAACAA |
| | | LEDGRTLSDYNIL | CGCCTTGCTTTTCAAGGGAAGAGCCTGGAGG |
| | | KDPKKMPLLRLR | ACGGTCGCACACTGTCTGACTATAACATTCTT |
| | | | AAAGATCCTAAAAAGATGCCACTGCTGCGCT |
| | | | TGCGT |
| CM49 | K6R, T7M, | MHHHHHHGGS | ATGCACCACCACCACCACCACGGTGGATCTG |
| [275; 925] | T12M, T14E, | GMLIFVRMLTGK | GCATGTTGATTTTCGTACGCATGTTGACTGGA |
| | K33H, S65P, | MIELEVEPSDTIE | AAGATGATCGAGTTGGAAGTGGAGCCTTCCG |
| | L67K, H68M | NVKAKIQDHEGI | ATACTATCGAGAATGTTAAGGCCAAAATCCA |
| | | PPDQQRLAFAGK | AGATCATGAAGGGATTCCTCCAGATCAACAA |
| | | SLEDGRTLSDYNI | CGCCTTGCTTTTGCCGGGAAGAGCCTGGAGG |
| | | LKDPKKMPLLRL | ACGGTCGCACACTGTCTGACTATAACATTCTT |
| | | R | AAAGATCCTAAAAAGATGCCACTGCTGCGCT |
| | | | TGCGT |
| CM50 | K6R, T7M, | MHHHHHHGGS | ATGCACCACCACCACCACCACGGTGGATCTG |
| [276; 926] | T12M, T14E, | GMLIFVRMLTGK | GCATGTTGATTTTCGTACGCATGTTGACTGGA |
| | K33H, A46Q, | MIELEVEPSDTIE | AAGATGATCGAGTTGGAAGTGGAGCCTTCCG |
| | L67K, H68M | NVKAKIQDHEGI | ATACTATCGAGAATGTTAAGGCCAAAATCCA |
| | | PPDQQRLAFQG | AGATCATGAAGGGATTCCTCCAGATCAACAA |
| | | KSLEDGRTLSDY | CGCCTTGCTTTTCAAGGGAAGAGCCTGGAGG |
| | | NILKDSKKMPLLR | ACGGTCGCACACTGTCTGACTATAACATTCTT |
| | | LR | AAAGATTCTAAAAAGATGCCACTGCTGCGCTT |
| | | | GCGT |
| CM51 | K6R, T7M, | MHHHHHHGGS | ATGCACCACCACCACCACCACGGTGGATCTG |
| [277; 927] | T12M, T14E, | GMLIFVRMLTGK | GCATGTTGATTTTCGTACGCATGTTGACTGGA |
| | K33H, A46Q, | MIELEVEPSDTIE | AAGATGATCGAGTTGGAAGTGGAGCCTTCCG |
| | S65P, H68M | NVKAKIQDHEGI | ATACTATCGAGAATGTTAAGGCCAAAATCCA |
| | | PPDQQRLAFQG | AGATCATGAAGGGATTCCTCCAGATCAACAA |
| | | KSLEDGRTLSDY | CGCCTTGCTTTTCAAGGGAAGAGCCTGGAGG |
| | | NILKDPKLMPLLR | ACGGTCGCACACTGTCTGACTATAACATTCTT |
| | | LR | AAAGATCCTAAATTGATGCCACTGCTGCGCTT |
| | | | GCGT |
| CM52 | K6R, T7M, | MHHHHHHGGS | ATGCACCACCACCACCACCACGGTGGATCTG |
| [278; 928] | T12M, T14E, | GMLIFVRMLTGK | GCATGTTGATTTTCGTACGCATGTTGACTGGA |
| | K33H, A46Q, | MIELEVEPSDTIE | AAGATGATCGAGTTGGAAGTGGAGCCTTCCG |
| | S65P, L67K | NVKAKIQDHEGI | ATACTATCGAGAATGTTAAGGCCAAAATCCA |
| | | PPDQQRLAFQG | AGATCATGAAGGGATTCCTCCAGATCAACAA |
| | | KSLEDGRTLSDY | CGCCTTGCTTTTCAAGGGAAGAGCCTGGAGG |
| | | NILKDPKKHPLLR | ACGGTCGCACACTGTCTGACTATAACATTCTT |
| | | LR | AAAGATCCTAAAAAGCATCCACTGCTGCGCTT |
| | | | GCGT |
| CM62 | K6R, T7M, | MHHHHHHGGS | ATGCACCACCACCACCACCACGGTGGATCTG |
| [279; 929] | T12M, T14E, | GHLIFVRMLTGK | GCCATTTGATTTTCGTACGCATGTTGACTGGA |
| | K33H, A46Q, | MIELEVEPSDTIE | AAGATGATCGAGTTGGAAGTGGAGCCTTCCG |
| | S65P, L67K, | NVKAKIQDHEGI | ATACTATCGAGAATGTTAAGGCCAAAATCCA |
| | H68M, M1H | PPDQQRLAFQG | AGATCATGAAGGGATTCCTCCAGATCAACAA |
| | | KSLEDGRTLSDY | CGCCTTGCTTTTCAAGGGAAGAGCCTGGAGG |
| | | NILKDPKKMPLLR | ACGGTCGCACACTGTCTGACTATAACATTCTT |
| | | LR | AAAGATCCTAAAAAGATGCCACTGCTGCGCT |
| | | | TGCGT |
| CM63 | K6R, T7M, | MHHHHHHGGS | ATGCACCACCACCACCACCACGGTGGATCTG |
| [280; 930] | T12M, T14E, | GYLIFVRMLTGK | GCTATTTGATTTTCGTACGCATGTTGACTGGA |
| | K33H, A46Q, | MIELEVEPSDTIE | AAGATGATCGAGTTGGAAGTGGAGCCTTCCG |
| | S65P, L67K, | NVKAKIQDHEGI | ATACTATCGAGAATGTTAAGGCCAAAATCCA |
| | H68M, M1Y | PPDQQRLAFQG | AGATCATGAAGGGATTCCTCCAGATCAACAA |
| | | KSLEDGRTLSDY | CGCCTTGCTTTTCAAGGGAAGAGCCTGGAGG |
| | | NILKDPKKMPLLR | ACGGTCGCACACTGTCTGACTATAACATTCTT |
| | | LR | AAAGATCCTAAAAAGATGCCACTGCTGCGCT |
| | | | TGCGT |
| CM64 | K6R, T7M, | MHHHHHHGGS | ATGCACCACCACCACCACCACGGTGGATCTG |
| [281; 931] | T12M, T14H, | GMLIFVRMLTGK | GCATGTTGATTTTCGTACGCATGTTGACTGGA |
| | K33H, A46Q, | MIHLEVEPSDTIE | AAGATGATCCATTTGGAAGTGGAGCCTTCCG |
| | S65P, L67K, | NVKAKIQDHEGI | ATACTATCGAGAATGTTAAGGCCAAAATCCA |
| | H68M | PPDQQRLAFQG | AGATCATGAAGGGATTCCTCCAGATCAACAA |
| | | KSLEDGRTLSDY | CGCCTTGCTTTTCAAGGGAAGAGCCTGGAGG |
| | | NILKDPKKMPLLR | ACGGTCGCACACTGTCTGACTATAACATTCTT |
| | | LR | AAAGATCCTAAAAAGATGCCACTGCTGCGCT |
| | | | TGCGT |
| CM65 | K6R, T7M, | MHHHHHHGGS | ATGCACCACCACCACCACCACGGTGGATCTG |
| [282; 932] | T12M, T14D, | GMLIFVRMLTGK | GCATGTTGATTTTCGTACGCATGTTGACTGGA |
| | K33H, A46Q, | MIDLEVEPSDTIE | AAGATGATCGATTTGGAAGTGGAGCCTTCCG |
| | S65P, L67K, | NVKAKIQDHEGI | ATACTATCGAGAATGTTAAGGCCAAAATCCA |
| | H68M | PPDQQRLAFQG | AGATCATGAAGGGATTCCTCCAGATCAACAA |
| | | KSLEDGRTLSDY | CGCCTTGCTTTTCAAGGGAAGAGCCTGGAGG |
| | | NILKDPKKMPLLR | ACGGTCGCACACTGTCTGACTATAACATTCTT |
| | | LR | AAAGATCCTAAAAAGATGCCACTGCTGCGCT |
| | | | TGCGT |
| CM66 | K6R, T7M, | MHHHHHHGGS | ATGCACCACCACCACCACCACGGTGGATCTG |
| [283; 933] | T12M, T14E, | GMLIFVRMLTGK | GCATGTTGATTTTCGTACGCATGTTGACTGGA |
| | K33H, A46Q, | MIELMVEPSDTIE | AAGATGATCGAGTTGATGGTGGAGCCTTCCG |
| | S65P, L67K, | NVKAKIQDHEGI | ATACTATCGAGAATGTTAAGGCCAAAATCCA |
| | H68M, E16M | PPDQQRLAFQG | AGATCATGAAGGGATTCCTCCAGATCAACAA |
| | | KSLEDGRTLSDY | CGCCTTGCTTTTCAAGGGAAGAGCCTGGAGG |
| | | NILKDPKKMPLLR | ACGGTCGCACACTGTCTGACTATAACATTCTT |
| | | LR | AAAGATCCTAAAAAGATGCCACTGCTGCGCT |
| | | | TGCGT |
| CM67 | K6R, T7M, | MHHHHHHGGS | ATGCACCACCACCACCACCACGGTGGATCTG |
| [284; 934] | T12M, T14E, | GMLIFVRMLTGK | GCATGTTGATTTTCGTACGCATGTTGACTGGA |
| | K33H, A46Q, | MIELTVEPSDTIE | AAGATGATCGAGTTGACTGTGGAGCCTTCCG |
| | S65P, L67K, | NVKAKIQDHEGI | ATACTATCGAGAATGTTAAGGCCAAAATCCA |
| | H68M, E16T | PPDQQRLAFQG | AGATCATGAAGGGATTCCTCCAGATCAACAA |
| | | KSLEDGRTLSDY | CGCCTTGCTTTTCAAGGGAAGAGCCTGGAGG |
| | | NILKDPKKMPLLR | ACGGTCGCACACTGTCTGACTATAACATTCTT |
| | | LR | AAAGATCCTAAAAAGATGCCACTGCTGCGCT |
| | | | TGCGT |
| CM68 | K6R, T7M, | MHHHHHHGGS | ATGCACCACCACCACCACCACGGTGGATCTG |
| [285; 935] | T12M, T14E, | GMLIFVRMLTGK | GCATGTTGATTTTCGTACGCATGTTGACTGGA |
| | K33H, A46Q, | MIELEVMPSDTIE | AAGATGATCGAGTTGGAAGTGATGCCTTCCG |
| | S65P, L67K, | NVKAKIQDHEGI | ATACTATCGAGAATGTTAAGGCCAAAATCCA |
| | H68M, E18M | PPDQQRLAFQG | AGATCATGAAGGGATTCCTCCAGATCAACAA |
| | | KSLEDGRTLSDY | CGCCTTGCTTTTCAAGGGAAGAGCCTGGAGG |
| | | NILKDPKKMPLLR | ACGGTCGCACACTGTCTGACTATAACATTCTT |
| | | LR | AAAGATCCTAAAAAGATGCCACTGCTGCGCT |
| | | | TGCGT |
| CM69 | K6R, T7M, | MHHHHHHGGS | ATGCACCACCACCACCACCACGGTGGATCTG |
| [286; 936] | T12M, T14E, | GMLIFVRMLTGK | GCATGTTGATTTTCGTACGCATGTTGACTGGA |
| | K33H, A46Q, | MIELEVYPSDTIE | AAGATGATCGAGTTGGAAGTGTATCCTTCCG |
| | S65P, L67K, | NVKAKIQDHEGI | ATACTATCGAGAATGTTAAGGCCAAAATCCA |
| | H68M, E18Y | PPDQQRLAFQG | AGATCATGAAGGGATTCCTCCAGATCAACAA |
| | | KSLEDGRTLSDY | CGCCTTGCTTTTCAAGGGAAGAGCCTGGAGG |
| | | NILKDPKKMPLLR | ACGGTCGCACACTGTCTGACTATAACATTCTT |
| | | LR | AAAGATCCTAAAAAGATGCCACTGCTGCGCT |
| | | | TGCGT |
| CM70 | K6R, T7M, | MHHHHHHGGS | ATGCACCACCACCACCACCACGGTGGATCTG |
| [287; 937] | T12M, T14E, | GMLIFVRMLTGK | GCATGTTGATTTTCGTACGCATGTTGACTGGA |
| | K33H, A46Q, | MIELEVLPSDTIE | AAGATGATCGAGTTGGAAGTGTTGCCTTCCG |
| | S65P, L67K, | NVKAKIQDHEGI | ATACTATCGAGAATGTTAAGGCCAAAATCCA |
| | H68M, E18L | PPDQQRLAFQG | AGATCATGAAGGGATTCCTCCAGATCAACAA |
| | | KSLEDGRTLSDY | CGCCTTGCTTTTCAAGGGAAGAGCCTGGAGG |
| | | NILKDPKKMPLLR | ACGGTCGCACACTGTCTGACTATAACATTCTT |
| | | LR | AAAGATCCTAAAAAGATGCCACTGCTGCGCT |
| | | | TGCGT |
| CM71 | K6R, T7M, | MHHHHHHGGS | ATGCACCACCACCACCACCACGGTGGATCTG |
| [288; 938] | T12M, T14E, | GMLIFVRMLTGK | GCATGTTGATTTTCGTACGCATGTTGACTGGA |
| | K33H, A46Q, | MIELEVFPSDTIE | AAGATGATCGAGTTGGAAGTGTTCCCTTCCG |
| | S65P, L67K, | NVKAKIQDHEGI | ATACTATCGAGAATGTTAAGGCCAAAATCCA |
| | H68M, E18F | PPDQQRLAFQG | AGATCATGAAGGGATTCCTCCAGATCAACAA |
| | | KSLEDGRTLSDY | CGCCTTGCTTTTCAAGGGAAGAGCCTGGAGG |
| | | NILKDPKKMPLLR | ACGGTCGCACACTGTCTGACTATAACATTCTT |
| | | LR | AAAGATCCTAAAAAGATGCCACTGCTGCGCT |
| | | | TGCGT |
| CM72 | K6R, T7M, | MHHHHHHGGS | ATGCACCACCACCACCACCACGGTGGATCTG |
| [289; 939] | T12M, T14E, | GMLIFVRMLTGK | GCATGTTGATTTTCGTACGCATGTTGACTGGA |
| | K33H, A46Q, | MIELEVEPSDTIE | AAGATGATCGAGTTGGAAGTGGAGCCTTCCG |
| | S65P, L67K, | VVKAKIQDHEGI | ATACTATCGAGGTAGTTAAGGCCAAAATCCA |
| | H68M, N25V | PPDQQRLAFQG | AGATCATGAAGGGATTCCTCCAGATCAACAA |
| | | KSLEDGRTLSDY | CGCCTTGCTTTTCAAGGGAAGAGCCTGGAGG |
| | | NILKDPKKMPLLR | ACGGTCGCACACTGTCTGACTATAACATTCTT |
| | | LR | AAAGATCCTAAAAAGATGCCACTGCTGCGCT |
| | | | TGCGT |
| CM73 | K6R, T7M, | MHHHHHHGGS | ATGCACCACCACCACCACCACGGTGGATCTG |
| [290; 940] | T12M, T14E, | GMLIFVRMLTGK | GCATGTTGATTTTCGTACGCATGTTGACTGGA |
| | K33H, A46Q, | MIELEVEPSDTIE | AAGATGATCGAGTTGGAAGTGGAGCCTTCCG |
| | S65P, L67K, | EVKAKIQDHEGIP | ATACTATCGAGGAGGTTAAGGCCAAAATCCA |
| | H68M, N25E | PDQQRLAFQGKS | AGATCATGAAGGGATTCCTCCAGATCAACAA |
| | | LEDGRTLSDYNIL | CGCCTTGCTTTTCAAGGGAAGAGCCTGGAGG |
| | | KDPKKMPLLRLR | ACGGTCGCACACTGTCTGACTATAACATTCTT |
| | | | AAAGATCCTAAAAAGATGCCACTGCTGCGCT |
| | | | TGCGT |
| CM74 | K6R, T7M, | MHHHHHHGGS | ATGCACCACCACCACCACCACGGTGGATCTG |
| [291; 941] | T12M, T14E, | GMLIFVRMLTGK | GCATGTTGATTTTCGTACGCATGTTGACTGGA |
| | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, V26I | PDQQRLAFQGKS | GATCATGAAGGGATTCCTCCAGATCAACAAC |
| | | LEDGRTLSDYNIL | GCCTTGCTTTTCAAGGGAAGAGCCTGGAGGA |
| | | KDPKKMPLLRLR | CGGTCGCACACTGTCTGACTATAACATTCTTA |
| | | | AAGATCCTAAAAAGATGCCACTGCTGCGCTT |
| | | | GCGT |
| CM75 | K6R, T7M, | MHHHHHHGGS | ATGCACCACCACCACCACCACGGTGGATCTG |
| [292; 942] | T12M, T14E, | GMLIFVRMLTGK | GCATGTTGATTTTCGTACGCATGTTGACTGGA |
| | K33H, A46Q, | MIELEVEPSDTIE | AAGATGATCGAGTTGGAAGTGGAGCCTTCCG |
| | S65P, L67K, | NVKAKICDHEGIP | ATACTATCGAGAATGTTAAGGCCAAAATCTG |
| | H68M, Q31C | PDQQRLAFQGKS | CGATCATGAAGGGATTCCTCCAGATCAACAA |
| | | LEDGRTLSDYNIL | CGCCTTGCTTTTCAAGGGAAGAGCCTGGAGG |
| | | KDPKKMPLLRLR | ACGGTCGCACACTGTCTGACTATAACATTCTT |
| | | | AAAGATCCTAAAAAGATGCCACTGCTGCGCT |
| | | | TGCGT |
| CM76 | K6R, T7M, | MHHHHHHGGS | ATGCACCACCACCACCACCACGGTGGATCTG |
| [293; 943] | T12M, T14E, | GMLIFVRMLTGK | GCATGTTGATTTTCGTACGCATGTTGACTGGA |
| | K33H, A46Q, | MIELEVEPSDTIE | AAGATGATCGAGTTGGAAGTGGAGCCTTCCG |
| | S65P, L67K, | NVKAKIWDHEGI | ATACTATCGAGAATGTTAAGGCCAAAATCTG |
| | H68M, Q31W | PPDQQRLAFQG | GGATCATGAAGGGATTCCTCCAGATCAACAA |
| | | KSLEDGRTLSDY | CGCCTTGCTTTTCAAGGGAAGAGCCTGGAGG |
| | | NILKDPKKMPLLR | ACGGTCGCACACTGTCTGACTATAACATTCTT |
| | | LR | AAAGATCCTAAAAAGATGCCACTGCTGCGCT |
| | | | TGCGT |
| CM77 | K6R, T7M, | MHHHHHHGGS | ATGCACCACCACCACCACCACGGTGGATCTG |
| [294; 944] | T12M, T14E, | GMLIFVRMLTGK | GCATGTTGATTTTCGTACGCATGTTGACTGGA |
| | K33H, A46Q, | MIELEVEPSDTIE | AAGATGATCGAGTTGGAAGTGGAGCCTTCCG |
| | S65P, L67K, | NVKAKIFDHEGIP | ATACTATCGAGAATGTTAAGGCCAAAATCTTC |
| | H68M, Q31F | PDQQRLAFQGKS | GATCATGAAGGGATTCCTCCAGATCAACAAC |
| | | LEDGRTLSDYNIL | GCCTTGCTTTTCAAGGGAAGAGCCTGGAGGA |
| | | KDPKKMPLLRLR | CGGTCGCACACTGTCTGACTATAACATTCTTA |
| | | | AAGATCCTAAAAAGATGCCACTGCTGCGCTT |
| | | | GCGT |
| CM78 | K6R, T7M, | MHHHHHHGGS | ATGCACCACCACCACCACCACGGTGGATCTG |
| [295; 945] | T12M, T14E, | GMLIFVRMLTGK | GCATGTTGATTTTCGTACGCATGTTGACTGGA |
| | K33H, A46Q, | MIELEVEPSDTIE | AAGATGATCGAGTTGGAAGTGGAGCCTTCCG |
| | S65P, L67K, | NVKAKIQAHEGI | ATACTATCGAGAATGTTAAGGCCAAAATCCA |
| | H68M, D32A | PPDQQRLAFQG | AGCCCATGAAGGGATTCCTCCAGATCAACAA |
| | | KSLEDGRTLSDY | CGCCTTGCTTTTCAAGGGAAGAGCCTGGAGG |
| | | NILKDPKKMPLLR | ACGGTCGCACACTGTCTGACTATAACATTCTT |
| | | LR | AAAGATCCTAAAAAGATGCCACTGCTGCGCT |
| | | | TGCGT |
| CM79 | K6R, T7M, | MHHHHHHGGS | ATGCACCACCACCACCACCACGGTGGATCTG |
| [296; 946] | T12M, T14E, | GMLIFVRMLTGK | GCATGTTGATTTTCGTACGCATGTTGACTGGA |
| | K33S, A46Q, | MIELEVEPSDTIE | AAGATGATCGAGTTGGAAGTGGAGCCTTCCG |
| | S65P, L67K, | NVKAKIQDSEGIP | ATACTATCGAGAATGTTAAGGCCAAAATCCA |
| | H68M | PDQQRLAFQGKS | AGATTCTGAAGGGATTCCTCCAGATCAACAA |
| | | LEDGRTLSDYNIL | CGCCTTGCTTTTCAAGGGAAGAGCCTGGAGG |
| | | KDPKKMPLLRLR | ACGGTCGCACACTGTCTGACTATAACATTCTT |
| | | | AAAGATCCTAAAAAGATGCCACTGCTGCGCT |
| | | | TGCGT |
| CM80 | K6R, T7M, | MHHHHHHGGS | ATGCACCACCACCACCACCACGGTGGATCTG |
| [297; 947] | T12M, T14E, | GMLIFVRMLTGK | GCATGTTGATTTTCGTACGCATGTTGACTGGA |
| | K33Q, A46Q, | MIELEVEPSDTIE | AAGATGATCGAGTTGGAAGTGGAGCCTTCCG |
| | S65P, L67K, | NVKAKIQDQEGI | ATACTATCGAGAATGTTAAGGCCAAAATCCA |
| | H68M | PPDQQRLAFQG | AGATCAAGAAGGGATTCCTCCAGATCAACAA |
| | | KSLEDGRTLSDY | CGCCTTGCTTTTCAAGGGAAGAGCCTGGAGG |
| | | NILKDPKKMPLLR | ACGGTCGCACACTGTCTGACTATAACATTCTT |
| | | LR | AAAGATCCTAAAAAGATGCCACTGCTGCGCT |
| | | | TGCGT |
| CM81 | K6R, T7M, | MHHHHHHGGS | ATGCACCACCACCACCACCACGGTGGATCTG |
| [298; 948] | T12M, T14E, | GMLIFVRMLTGK | GCATGTTGATTTTCGTACGCATGTTGACTGGA |
| | K33A, A46Q, | MIELEVEPSDTIE | AAGATGATCGAGTTGGAAGTGGAGCCTTCCG |
| | S65P, L67K, | NVKAKIQDAEGI | ATACTATCGAGAATGTTAAGGCCAAAATCCA |
| | H68M | PPDQQRLAFQG | AGATGCCGAAGGGATTCCTCCAGATCAACAA |
| | | KSLEDGRTLSDY | CGCCTTGCTTTTCAAGGGAAGAGCCTGGAGG |
| | | NILKDPKKMPLLR | ACGGTCGCACACTGTCTGACTATAACATTCTT |
| | | LR | AAAGATCCTAAAAAGATGCCACTGCTGCGCT |
| | | | TGCGT |
| CM82 | K6R, T7M, | MHHHHHHGGS | ATGCACCACCACCACCACCACGGTGGATCTG |
| [299; 949] | T12M, T14E, | GMLIFVRMLTGK | GCATGTTGATTTTCGTACGCATGTTGACTGGA |
| | K33H, A46Q, | MIELEVEPSDTIE | AAGATGATCGAGTTGGAAGTGGAGCCTTCCG |
| | S65P, L67K, | NVKAKIQDHEGI | ATACTATCGAGAATGTTAAGGCCAAAATCCA |
| | H68M, P38L | PLDQQRLAFQGK | AGATCATGAAGGGATTCCTTTGGATCAACAA |
| | | SLEDGRTLSDYNI | CGCCTTGCTTTTCAAGGGAAGAGCCTGGAGG |
| | | LKDPKKMPLLRL | ACGGTCGCACACTGTCTGACTATAACATTCTT |
| | | R | AAAGATCCTAAAAAGATGCCACTGCTGCGCT |
| | | | TGCGT |
| CM83 | K6R, T7M, | MHHHHHHGGS | ATGCACCACCACCACCACCACGGTGGATCTG |
| [300; 950] | T12M, T14E, | GMLIFVRMLTGK | GCATGTTGATTTTCGTACGCATGTTGACTGGA |
| | K33H, A46Q, | MIELEVEPSDTIE | AAGATGATCGAGTTGGAAGTGGAGCCTTCCG |
| | S65P, L67K, | NVKAKIQDHEGI | ATACTATCGAGAATGTTAAGGCCAAAATCCA |
| | H68M, P38C | PCDQQRLAFQG | AGATCATGAAGGGATTCCTTGCGATCAACAA |
| | | KSLEDGRTLSDY | CGCCTTGCTTTTCAAGGGAAGAGCCTGGAGG |
| | | NILKDPKKMPLLR | ACGGTCGCACACTGTCTGACTATAACATTCTT |
| | | LR | AAAGATCCTAAAAAGATGCCACTGCTGCGCT |
| | | | TGCGT |
| CM84 | K6R, T7M, | MHHHHHHGGS | ATGCACCACCACCACCACCACGGTGGATCTG |
| [301; 951] | T12M, T14E, | GMLIFVRMLTGK | GCATGTTGATTTTCGTACGCATGTTGACTGGA |
| | K33H, A46Q, | MIELEVEPSDTIE | AAGATGATCGAGTTGGAAGTGGAGCCTTCCG |
| | S65P, L67K, | NVKAKIQDHEGI | ATACTATCGAGAATGTTAAGGCCAAAATCCA |
| | H68M, Q40E | PPDEQRLAFQGK | AGATCATGAAGGGATTCCTCCAGATGAGCAA |
| | | SLEDGRTLSDYNI | CGCCTTGCTTTTCAAGGGAAGAGCCTGGAGG |
| | | LKDPKKMPLLRL | ACGGTCGCACACTGTCTGACTATAACATTCTT |
| | | R | AAAGATCCTAAAAAGATGCCACTGCTGCGCT |
| | | | TGCGT |
| CM87 | K6R, T7M, | MHHHHHHGGS | ATGCACCACCACCACCACCACGGTGGATCTG |
| [302; 952] | T12M, T14E, | GMLIFVRMLTGK | GCATGTTGATTTTCGTACGCATGTTGACTGGA |
| | K33H, A46Q, | MIELEVEPSDTIE | AAGATGATCGAGTTGGAAGTGGAGCCTTCCG |
| | S65P, L67K, | NVKAKIQDHEGI | ATACTATCGAGAATGTTAAGGCCAAAATCCA |
| | H68M, R42H | PPDQQHLAFQG | AGATCATGAAGGGATTCCTCCAGATCAACAA |
| | | KSLEDGRTLSDY | CATCTTGCTTTTCAAGGGAAGAGCCTGGAGG |
| | | NILKDPKKMPLLR | ACGGTCGCACACTGTCTGACTATAACATTCTT |
| | | LR | AAAGATCCTAAAAAGATGCCACTGCTGCGCT |
| | | | TGCGT |
| CM88 | K6R, T7M, | MHHHHHHGGS | ATGCACCACCACCACCACCACGGTGGATCTG |
| [303; 953] | T12M, T14E, | GMLIFVRMLTGK | GCATGTTGATTTTCGTACGCATGTTGACTGGA |
| | K33H, A46Q, | MIELEVEPSDTIE | AAGATGATCGAGTTGGAAGTGGAGCCTTCCG |
| | S65P, L67K, | NVKAKIQDHEGI | ATACTATCGAGAATGTTAAGGCCAAAATCCA |
| | H68M, R42F | PPDQQFLAFQGK | AGATCATGAAGGGATTCCTCCAGATCAACAA |
| | | SLEDGRTLSDYNI | TTCCTTGCTTTTCAAGGGAAGAGCCTGGAGG |
| | | LKDPKKMPLLRL | ACGGTCGCACACTGTCTGACTATAACATTCTT |
| | | R | AAAGATCCTAAAAAGATGCCACTGCTGCGCT |
| | | | TGCGT |
| CM89 | K6R, T7M, | MHHHHHHGGS | ATGCACCACCACCACCACCACGGTGGATCTG |
| [304; 954] | T12M, T14E, | GMLIFVRMLTGK | GCATGTTGATTTTCGTACGCATGTTGACTGGA |
| | K33H, A46Q, | MIELEVEPSDTIE | AAGATGATCGAGTTGGAAGTGGAGCCTTCCG |
| | S65P, L67K, | NVKAKIQDHEGI | ATACTATCGAGAATGTTAAGGCCAAAATCCA |
| | H68M, A44T | PPDQQRLTFQGK | AGATCATGAAGGGATTCCTCCAGATCAACAA |
| | | SLEDGRTLSDYNI | CGCCTTACTTTTCAAGGGAAGAGCCTGGAGG |
| | | LKDPKKMPLLRL | ACGGTCGCACACTGTCTGACTATAACATTCTT |
| | | R | AAAGATCCTAAAAAGATGCCACTGCTGCGCT |
| | | | TGCGT |
| CM90 | K6R, T7M, | MHHHHHHGGS | ATGCACCACCACCACCACCACGGTGGATCTG |
| [305; 955] | T12M, T14E, | GMLIFVRMLTGK | GCATGTTGATTTTCGTACGCATGTTGACTGGA |
| | K33H, A46Q, | MIELEVEPSDTIE | AAGATGATCGAGTTGGAAGTGGAGCCTTCCG |
| | S65P, L67K, | NVKAKIQDHEGI | ATACTATCGAGAATGTTAAGGCCAAAATCCA |
| | H68M, K48T | PPDQQRLAFQGT | AGATCATGAAGGGATTCCTCCAGATCAACAA |
| | | SLEDGRTLSDYNI | CGCCTTGCTTTTCAAGGGACTAGCCTGGAGG |
| | | LKDPKKMPLLRL | ACGGTCGCACACTGTCTGACTATAACATTCTT |
| | | R | AAAGATCCTAAAAAGATGCCACTGCTGCGCT |
| | | | TGCGT |
| CM92 | K6R, T7M, | MHHHHHHGGS | ATGCACCACCACCACCACCACGGTGGATCTG |
| [306; 956] | T12M, T14E, | GMLIFVRMLTGK | GCATGTTGATTTTCGTACGCATGTTGACTGGA |
| | K33H, A46Q, | MIELEVEPSDTIE | AAGATGATCGAGTTGGAAGTGGAGCCTTCCG |
| | S65P, L67K, | NVKAKIQDHEGI | ATACTATCGAGAATGTTAAGGCCAAAATCCA |
| | H68M, S49L | PPDQQRLAFQG | AGATCATGAAGGGATTCCTCCAGATCAACAA |
| | | KLLEDGRTLSDYN | CGCCTTGCTTTTCAAGGGAAGTTGCTGGAGG |
| | | ILKDPKKMPLLRL | ACGGTCGCACACTGTCTGACTATAACATTCTT |
| | | R | AAAGATCCTAAAAAGATGCCACTGCTGCGCT |
| | | | TGCGT |
| CM93 | K6R, T7M, | MHHHHHHGGS | ATGCACCACCACCACCACCACGGTGGATCTG |
| [307; 957] | T12M, T14E, | GMLIFVRMLTGK | GCATGTTGATTTTCGTACGCATGTTGACTGGA |
| | K33H, A46Q, | MIELEVEPSDTIE | AAGATGATCGAGTTGGAAGTGGAGCCTTCCG |
| | S65P, L67K, | NVKAKIQDHEGI | ATACTATCGAGAATGTTAAGGCCAAAATCCA |
| | H68M, S49M | PPDQQRLAFQG | AGATCATGAAGGGATTCCTCCAGATCAACAA |
| | | KMLEDGRTLSDY | CGCCTTGCTTTTCAAGGGAAGATGCTGGAGG |
| | | NILKDPKKMPLLR | ACGGTCGCACACTGTCTGACTATAACATTCTT |
| | | LR | AAAGATCCTAAAAAGATGCCACTGCTGCGCT |
| | | | TGCGT |
| CM94 | K6R, T7M, | MHHHHHHGGS | ATGCACCACCACCACCACCACGGTGGATCTG |
| [308; 958] | T12M, T14E, | GMLIFVRMLTGK | GCATGTTGATTTTCGTACGCATGTTGACTGGA |
| | K33H, A46Q, | MIELEVEPSDTIE | AAGATGATCGAGTTGGAAGTGGAGCCTTCCG |
| | S65P, L67K, | NVKAKIQDHEGI | ATACTATCGAGAATGTTAAGGCCAAAATCCA |
| | H68M, E51D | PPDQQRLAFQG | AGATCATGAAGGGATTCCTCCAGATCAACAA |
| | | KSLDDGRTLSDY | CGCCTTGCTTTTCAAGGGAAGAGCCTGGATG |
| | | NILKDPKKMPLLR | ACGGTCGCACACTGTCTGACTATAACATTCTT |
| | | LR | AAAGATCCTAAAAAGATGCCACTGCTGCGCT |
| | | | TGCGT |
| CM95 | K6R, T7M, | MHHHHHHGGS | ATGCACCACCACCACCACCACGGTGGATCTG |
| [309; 959] | T12M, T14E, | GMLIFVRMLTGK | GCATGTTGATTTTCGTACGCATGTTGACTGGA |
| | K33H, A46Q, | MIELEVEPSDTIE | AAGATGATCGAGTTGGAAGTGGAGCCTTCCG |
| | S65P, L67K, | NVKAKIQDHEGI | ATACTATCGAGAATGTTAAGGCCAAAATCCA |
| | H68M, R54Y | PPDQQRLAFQG | AGATCATGAAGGGATTCCTCCAGATCAACAA |
| | | KSLEDGYTLSDYN | CGCCTTGCTTTTCAAGGGAAGAGCCTGGAGG |
| | | ILKDPKKMPLLRL | ACGGTTATACACTGTCTGACTATAACATTCTT |
| | | R | AAAGATCCTAAAAAGATGCCACTGCTGCGCT |
| | | | TGCGT |
| CM98 | K6R, T7M, | MHHHHHHGGS | ATGCACCACCACCACCACCACGGTGGATCTG |
| [310; 960] | T12M, T14E, | GMLIFVRMLTGK | GCATGTTGATTTTCGTACGCATGTTGACTGGA |
| | K33H, A46Q, | MIELEVEPSDTIE | AAGATGATCGAGTTGGAAGTGGAGCCTTCCG |
| | S65P, L67K, | NVKAKIQDHEGI | ATACTATCGAGAATGTTAAGGCCAAAATCCA |
| | H68M, S57G | PPDQQRLAFQG | AGATCATGAAGGGATTCCTCCAGATCAACAA |
| | | KSLEDGRTLGDY | CGCCTTGCTTTTCAAGGGAAGAGCCTGGAGG |
| | | NILKDPKKMPLLR | ACGGTCGCACACTGGGGGACTATAACATTCT |
| | | LR | TAAAGATCCTAAAAAGATGCCACTGCTGCGC |
| | | | TTGCGT |
| CM101 | K6R, T7M, | MHHHHHHGGS | ATGCACCACCACCACCACCACGGTGGATCTG |
| [311; 961] | T12M, T14E, | GMLIFVRMLTGK | GCATGTTGATTTTCGTACGCATGTTGACTGGA |
| | K33H, A46Q, | MIELEVEPSDTIE | AAGATGATCGAGTTGGAAGTGGAGCCTTCCG |
| | S65P, L67K, | NVKAKIQDHEGI | ATACTATCGAGAATGTTAAGGCCAAAATCCA |
| | H68M, I61L | PPDQQRLAFQG | AGATCATGAAGGGATTCCTCCAGATCAACAA |
| | | KSLEDGRTLSDY | CGCCTTGCTTTTCAAGGGAAGAGCCTGGAGG |
| | | NLLKDPKKMPLL | ACGGTCGCACACTGTCTGACTATAACTTGCTT |
| | | RLR | AAAGATCCTAAAAAGATGCCACTGCTGCGCT |
| | | | TGCGT |
| CM102 | K6R, T7M, | MHHHHHHGGS | ATGCACCACCACCACCACCACGGTGGATCTG |
| [312; 962] | T12M, T14E, | GMLIFVRMLTGK | GCATGTTGATTTTCGTACGCATGTTGACTGGA |
| | K33H, A46Q, | MIELEVEPSDTIE | AAGATGATCGAGTTGGAAGTGGAGCCTTCCG |
| | S65P, L67K, | NVKAKIQDHEGI | ATACTATCGAGAATGTTAAGGCCAAAATCCA |
| | H68M, K63I | PPDQQRLAFQG | AGATCATGAAGGGATTCCTCCAGATCAACAA |
| | | KSLEDGRTLSDY | CGCCTTGCTTTTCAAGGGAAGAGCCTGGAGG |
| | | NILIDPKKMPLLR | ACGGTCGCACACTGTCTGACTATAACATTCTT |
| | | LR | ATTGATCCTAAAAAGATGCCACTGCTGCGCTT |
| | | | GCGT |
| CM103 | K6R, T7M, | MHHHHHHGGS | ATGCACCACCACCACCACCACGGTGGATCTG |
| [313; 963] | T12M, T14E, | GMLIFVRMLTGK | GCATGTTGATTTTCGTACGCATGTTGACTGGA |
| | K33H, A46Q, | MIELEVEPSDTIE | AAGATGATCGAGTTGGAAGTGGAGCCTTCCG |
| | S65H, L67K, | NVKAKIQDHEGI | ATACTATCGAGAATGTTAAGGCCAAAATCCA |
| | H68M | PPDQQRLAFQG | AGATCATGAAGGGATTCCTCCAGATCAACAA |
| | | KSLEDGRTLSDY | CGCCTTGCTTTTCAAGGGAAGAGCCTGGAGG |
| | | NILKDHKKMPLL | ACGGTCGCACACTGTCTGACTATAACATTCTT |
| | | RLR | AAAGATCATAAAAAGATGCCACTGCTGCGCT |
| | | | TGCGT |
| CM104 | K6R, T7M, | MHHHHHHGGS | ATGCACCACCACCACCACCACGGTGGATCTG |
| [314; 964] | T12M, T14E, | GMLIFVRMLTGK | GCATGTTGATTTTCGTACGCATGTTGACTGGA |
| | K33H, A46Q, | MIELEVEPSDTIE | AAGATGATCGAGTTGGAAGTGGAGCCTTCCG |
| | S65P, L67K, | NVKAKIQDHEGI | ATACTATCGAGAATGTTAAGGCCAAAATCCA |
| | H68M, L73M | PPDQQRLAFQG | AGATCATGAAGGGATTCCTCCAGATCAACAA |
| | | KSLEDGRTLSDY | CGCCTTGCTTTTCAAGGGAAGAGCCTGGAGG |
| | | NILKDPKKMPLLR | ACGGTCGCACACTGTCTGACTATAACATTCTT |
| | | MR | AAAGATCCTAAAAAGATGCCACTGCTGCGCA |
| | | | TGCGT |
| CM105 | K6R, T7M, | MHHHHHHGGS | ATGCACCACCACCACCACCACGGTGGATCTG |
| [315; 965] | T12M, T14E, | GMLIFVRMLTGK | GCATGTTGATTTTCGTACGCATGTTGACTGGA |
| | K33H, A46Q, | MIELEVEPSDTIE | AAGATGATCGAGTTGGAAGTGGAGCCTTCCG |
| | S65P, L67K, | NVKAKIQDHEGI | ATACTATCGAGAATGTTAAGGCCAAAATCCA |
| | H68M, R74Q | PPDQQRLAFQG | AGATCATGAAGGGATTCCTCCAGATCAACAA |
| | | KSLEDGRTLSDY | CGCCTTGCTTTTCAAGGGAAGAGCCTGGAGG |
| | | NILKDPKKMPLLR | ACGGTCGCACACTGTCTGACTATAACATTCTT |
| | | LQ | AAAGATCCTAAAAAGATGCCACTGCTGCGCT |
| | | | TGCAA |
| CM107 | T7M, T12M, | MHHHHHHGGS | ATGCACCACCACCACCACCACGGTGGATCTG |
| [316; 966] | T14E, K33H, | GMLIFVKMLTGK | GCATGTTGATTTTCGTACGCATGTTGACTGGA |
| | A46Q, S65P, | MIELEVEPSDTIE | AAGATGATCGAGTTGGAAGTGGAGCCTTCCG |
| | L67K, H68M, | NVKAKIQDHEGI | ATACTATCGAGAATGTTAAGGCCAAAATCCA |
| | P69L, L70V | PPDQQRLAFQG | AGATCATGAAGGGATTCCTCCAGATCAACAA |
| | | KSLEDGRTLSDY | CGCCTTGCTTTTCAAGGGAAGAGCCTGGAGG |
| | | NILKDPKKMLVL | ACGGTCGCACACTGTCTGACTATAACATTCTT |
| | | RLR | AAAGATCCTAAAAAGATGTTGGTACTGCGCT |
| | | | TGCGT |
| CM108 | K6R, T7M, | MHHHHHHGGS | ATGCACCACCACCACCACCACGGTGGATCTG |
| [317; 967] | T12M, T14E, | GMLIFVRMLTGK | GCATGTTGATTTTCGTACGCATGTTGACTGGA |
| | K33H, A46Q, | MIELEVYPSDTIE | AAGATGATCGAGTTGGAAGTGTATCCTTCCG |
| | S65P, L67K, | NVKAKIQDHEGI | ATACTATCGAGAATGTTAAGGCCAAAATCCA |
| | H68M, E18Y, | PLDQQRLAFQGK | AGATCATGAAGGGATTCCTTTGGATCAACAA |
| | P38L, S49L, | LLEDGRTLGDYNI | CGCCTTGCTTTTCAAGGGAAGTTGCTGGAGG |
| | S57G | LKDPKKMPLLRL | ACGGTCGCACACTGGGGGACTATAACATTCT |
| | | R | TAAAGATCCTAAAAAGATGCCACTGCTGCGC |
| | | | TTGCGT |
| CM110 | K6R, T7M, | MHHHHHHGGS | ATGCACCACCACCACCACCACGGTGGATCTG |
| [318; 968] | T12M, T14E, | GHLIFVRMLTGK | GCCATTTGATTTTCGTACGCATGTTGACTGGA |
| | K33H, A46Q, | MIELEVEPSDTIE | AAGATGATCGAGTTGGAAGTGGAGCCTTCCG |
| | S65P, L67K, | NVKAKIQDHEGI | ATACTATCGAGAATGTTAAGGCCAAAATCCA |
| | H68M, M1H, | PPDQQRLAFQG | AGATCATGAAGGGATTCCTCCAGATCAACAA |
| | R74Q | KSLEDGRTLSDY | CGCCTTGCTTTTCAAGGGAAGAGCCTGGAGG |
| | | NILKDPKKMPLLR | ACGGTCGCACACTGTCTGACTATAACATTCTT |
| | | LQ | AAAGATCCTAAAAAGATGCCACTGCTGCGCT |
| | | | TGCAA |
| CM111 | K6R, T7M, | MHHHHHHGGS | ATGCACCACCACCACCACCACGGTGGATCTG |
| [319; 969] | T12M, T14E, | GYLIFVRMLTGK | GCTATTTGATTTTCGTACGCATGTTGACTGGA |
| | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, M1Y, | PDQQRLAFQGKS | GATCATGAAGGGATTCCTCCAGATCAACAAC |
| | V26I, L73M | LEDGRTLSDYNIL | GCCTTGCTTTTCAAGGGAAGAGCCTGGAGGA |
| | | KDPKKMPLLRM | CGGTCGCACACTGTCTGACTATAACATTCTTA |
| | | R | AAGATCCTAAAAAGATGCCACTGCTGCGCAT |
| | | | GCGT |
| CM112 | K6R, T7M, | MHHHHHHGGS | ATGCACCACCACCACCACCACGGTGGATCTG |
| [320; 970] | T12M, T14E, | GMLIFVRMLTGK | GCATGTTGATTTTCGTACGCATGTTGACTGGA |
| | K33H, A46Q, | MIELEVEPSDTIE | AAGATGATCGAGTTGGAAGTGGAGCCTTCCG |
| | S65P, L67K, | VVKAKIQDHEGI | ATACTATCGAGGTAGTTAAGGCCAAAATCCA |
| | H68M, N25V, | PPDEQRLAFQGK | AGATCATGAAGGGATTCCTCCAGATGAGCAA |
| | Q40E, E51D | SLDDGRTLSDYNI | CGCCTTGCTTTTCAAGGGAAGAGCCTGGATG |
| | | LKDPKKMPLLRL | ACGGTCGCACACTGTCTGACTATAACATTCTT |
| | | R | AAAGATCCTAAAAAGATGCCACTGCTGCGCT |
| | | | TGCGT |
| CM113 | K6R, T7M, | MHHHHHHGGS | ATGCACCACCACCACCACCACGGTGGATCTG |
| [321; 971] | T12M, T14E, | GMLIFVRMLTGK | GCATGTTGATTTTCGTACGCATGTTGACTGGA |
| | K33H, A46Q, | MIELEVEPSDTIE | AAGATGATCGAGTTGGAAGTGGAGCCTTCCG |
| | S65P, L67K, | NVKAKIQDHEGI | ATACTATCGAGAATGTTAAGGCCAAAATCCA |
| | H68M, I61L, | PPDQQRLAFQG | AGATCATGAAGGGATTCCTCCAGATCAACAA |
| | K63I | KSLEDGRTLSDY | CGCCTTGCTTTTCAAGGGAAGAGCCTGGAGG |
| | | NLLIDPKKMPLLR | ACGGTCGCACACTGTCTGACTATAACTTGCTT |
| | | LR | ATTGATCCTAAAAAGATGCCACTGCTGCGCTT |
| | | | GCGT |
| CM114 | K6R, T7M, | MHHHHHHGGS | ATGCACCACCACCACCACCACGGTGGATCTG |
| [322; 972] | T12M, T14E, | GMLIFVRMLTGK | GCATGTTGATTTTCGTACGCATGTTGACTGGA |
| | K33H, A46Q, | MIELEVMPSDTIE | AAGATGATCGAGTTGGAAGTGATGCCTTCCG |
| | S65P, L67K, | NVKAKIQDHEGI | ATACTATCGAGAATGTTAAGGCCAAAATCCA |
| | H68M, E18M, | PPDQQRLAFQGT | AGATCATGAAGGGATTCCTCCAGATCAACAA |
| | K48T, E51D, | SLDDGRTLGDYN | CGCCTTGCTTTTCAAGGGACTAGCCTGGATGA |
| | S57G | ILKDPKKMPLLRL | CGGTCGCACACTGGGGGACTATAACATTCTT |
| | | R | AAAGATCCTAAAAAGATGCCACTGCTGCGCT |
| | | | TGCGT |
| CM115 | K6R, T7M, | MHHHHHHGGS | ATGCACCACCACCACCACCACGGTGGATCTG |
| [323; 973] | T12M, T14E, | GMLIFVRMLTGK | GCATGTTGATTTTCGTACGCATGTTGACTGGA |
| | K33H, A46Q, | MIELMVEPSDTIE | AAGATGATCGAGTTGATGGTGGAGCCTTCCG |
| | S65P, L67K, | VVKAKIQDHEGI | ATACTATCGAGGTAGTTAAGGCCAAAATCCA |
| | H68M, E16M, | PPDEQRLAFQGK | AGATCATGAAGGGATTCCTCCAGATGAGCAA |
| | N25V, Q40E, | LLEDGRTLSDYNI | CGCCTTGCTTTTCAAGGGAAGTTGCTGGAGG |
| | S49L | LKDPKKMPLLRL | ACGGTCGCACACTGTCTGACTATAACATTCTT |
| | | R | AAAGATCCTAAAAAGATGCCACTGCTGCGCT |
| | | | TGCGT |
| CM116 | K6R, T7M, | MHHHHHHGGS | ATGCACCACCACCACCACCACGGTGGATCTG |
| [324; 974] | T12M, T14E, | GMLIFVRMLTGK | GCATGTTGATTTTCGTACGCATGTTGACTGGA |
| | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, V26I, | PDQYRLAFQGKL | GATCATGAAGGGATTCCTCCAGATCAATATC |
| | Q41Y, S49L, | LEDGRTLGDYNIL | GCCTTGCTTTTCAAGGGAAGTTGCTGGAGGA |
| | S57G | KDPKKMPLLRLR | CGGTCGCACACTGGGGGACTATAACATTCTT |
| | | | AAAGATCCTAAAAAGATGCCACTGCTGCGCT |
| | | | TGCGT |
| CM117 | K6R, T7M, | MHHHHHHGGS | ATGCACCACCACCACCACCACGGTGGATCTG |
| [325; 975] | T12M, T14E, | GMLIFVRMLTGK | GCATGTTGATTTTCGTACGCATGTTGACTGGA |
| | K33H, A46Q, | MIELEVEPSDTIE | AAGATGATCGAGTTGGAAGTGGAGCCTTCCG |
| | S65P, L67K, | NVKAKICDHEGIP | ATACTATCGAGAATGTTAAGGCCAAAATCTG |
| | H68M, Q31C, | PDQQHLAFQGK | CGATCATGAAGGGATTCCTCCAGATCAACAA |
| | R42H, S57G | SLEDGRTLGDYNI | CATCTTGCTTTTCAAGGGAAGAGCCTGGAGG |
| | | LKDPKKMPLLRL | ACGGTCGCACACTGGGGGACTATAACATTCT |
| | | R | TAAAGATCCTAAAAAGATGCCACTGCTGCGC |
| | | | TTGCGT |
| CM118 | K6R, T7M, | MHHHHHHGGS | ATGCACCACCACCACCACCACGGTGGATCTG |
| [326; 976] | T12M, T14E, | GMLIFVRMLTGK | GCATGTTGATTTTCGTACGCATGTTGACTGGA |
| | K33H, A46Q, | MIELEVMPSDTIE | AAGATGATCGAGTTGGAAGTGATGCCTTCCG |
| | S65P, L67K, | WVVAKIFDHEGIP | ATACTATCGAGGTAGTTAAGGCCAAAATCTTC |
| | H68M, E18M, | PDQQHLAFQGT | GATCATGAAGGGATTCCTCCAGATCAACAAC |
| | N25V, Q31F, | SLEDGYTLGDYNI | ATCTTGCTTTTCAAGGGACTAGCCTGGAGGA |
| | R42H, K48T, | LKDPKKMPLLRL | CGGTTATACACTGGGGGACTATAACATTCTTA |
| | R54Y, S57G | R | AAGATCCTAAAAAGATGCCACTGCTGCGCTT |
| | | | GCGT |
| CM119 | K6R, T7M, | MHHHHHHGGS | ATGCACCACCACCACCACCACGGTGGATCTG |
| [327; 977] | T12M, T14E, | GMLIFVRMLTGK | GCATGTTGATTTTCGTACGCATGTTGACTGGA |
| | K33H, A46Q, | MIELEVMPSDTIE | AAGATGATCGAGTTGGAAGTGATGCCTTCCG |
| | S65P, L67K, | VVKAKIFDHEGIP | ATACTATCGAGGTAGTTAAGGCCAAAATCTTC |
| | H68M, E18M, | PDQQHLTFQGTL | GATCATGAAGGGATTCCTCCAGATCAACAAC |
| | N25V, Q31F, | LEDGYTLGDYNIL | ATCTTACTTTTCAAGGGACTTTGCTGGAGGAC |
| | R42H, A44T, | KDPKKMPLLRLR | GGTTATACACTGGGGGACTATAACATTCTTAA |
| | K48T, S49L, | | AGATCCTAAAAAGATGCCACTGCTGCGCTTG |
| | R54Y, S57G | | CGT |
| CM120 | K6R, T7M, | MHHHHHHGGS | ATGCACCACCACCACCACCACGGTGGATCTG |
| [328; 978] | T12M, T14E, | GMLIFVRMLTGK | GCATGTTGATTTTCGTACGCATGTTGACTGGA |
| | K33H, A46Q, | MIELEVMPSDTIE | AAGATGATCGAGTTGGAAGTGATGCCTTCCG |
| | S65P, L67K, | VVKAKIFDHEGIP | ATACTATCGAGGTAGTTAAGGCCAAAATCTTC |
| | H68M, E18M, | LDQQHLAFQGTS | GATCATGAAGGGATTCCTTTGGATCAACAAC |
| | N25V, Q31F, | LEDGYTLGDYNIL | ATCTTGCTTTTCAAGGGACTAGCCTGGAGGA |
| | P38L, R42H, | KDPKKMPLLRLR | CGGTTATACACTGGGGGACTATAACATTCTTA |
| | K48T, R54Y, | | AAGATCCTAAAAAGATGCCACTGCTGCGCTT |
| | S57G | | GCGT |
| CM121 | K6R, T7M, | MHHHHHHGGS | ATGCACCACCACCACCACCACGGTGGATCTG |
| [329; 979] | T12M, T14E, | GMLIFVRMLTGK | GCATGTTGATTTTCGTACGCATGTTGACTGGA |
| | K33H, A46Q, | MIELEVMPSDTIE | AAGATGATCGAGTTGGAAGTGATGCCTTCCG |
| | S65P, L67K, | VVKAKIQDHEGI | ATACTATCGAGGTAGTTAAGGCCAAAATCCA |
| | H68M, E18M, | PPDQQHLAFQG | AGATCATGAAGGGATTCCTCCAGATCAACAA |
| | N25V, R42H, | TSLEDGYTLGDY | CATCTTGCTTTTCAAGGGACTAGCCTGGAGG |
| | K48T, R54Y, | NILKDPKKMPLLR | ACGGTTATACACTGGGGGACTATAACATTCTT |
| | S57G | LR | AAAGATCCTAAAAAGATGCCACTGCTGCGCT |
| | | | TGCGT |
| CM131 | K6R, T7M, | MHHHHHHGGS | ATGCACCACCACCACCACCACGGTGGATCTG |
| [330; 980] | T12M, T14E, | GYLIFVRMLTGK | GCTATTTGATTTTCGTACGCATGTTGACTGGA |
| | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | PDQQRLAFQGKS | GATCATGAAGGGATTCCTCCAGATCAACAAC |
| | K63I, M1Y, | LEDGRTLSDYNLL | GCCTTGCTTTTCAAGGGAAGAGCCTGGAGGA |
| | V26I, L73M | IDPKKMPLLRMR | CGGTCGCACACTGTCTGACTATAACTTGCTTA |
| | | | TTGATCCTAAAAAGATGCCACTGCTGCGCATG |
| | | | CGT |
| CM132 | K6R, T7M, | MHHHHHHGGS | ATGCACCACCACCACCACCACGGTGGATCTG |
| [331; 981] | T12M, T14E, | GYLIFVRMLTGK | GCTATTTGATTTTCGTACGCATGTTGACTGGA |
| | K33H, A46Q, | MIELEVEPSDTIE | AAGATGATCGAGTTGGAAGTGGAGCCTTCCG |
| | S65P, L67K, | VIKAKIQDHEGIP | ATACTATCGAGGTAATTAAGGCCAAAATCCA |
| | H68M, I61L, | PDEQRLAFQGKS | AGATCATGAAGGGATTCCTCCAGATGAGCAA |
| | K63I, M1Y, | LDDGRTLSDYNLL | CGCCTTGCTTTTCAAGGGAAGAGCCTGGATG |
| | V26I, L73M, | IDPKKMPLLRMR | ACGGTCGCACACTGTCTGACTATAACTTGCTT |
| | N25V, Q40E, | | ATTGATCCTAAAAAGATGCCACTGCTGCGCAT |
| | E51D | | GCGT |
| CM133 | K6R, T7M, | MHHHHHHGGS | ATGCACCACCACCACCACCACGGTGGATCTG |
| [332; 982] | T12M, T14E, | GMLIFVRMLTGK | GCATGTTGATTTTCGTACGCATGTTGACTGGA |
| | K33H, A46Q, | MIELEVEPSDTIE | AAGATGATCGAGTTGGAAGTGGAGCCTTCCG |
| | S65P, L67K, | VVKAKIQDHEGI | ATACTATCGAGGTAGTTAAGGCCAAAATCCA |
| | H68M, I61L, | PPDEQRLAFQGK | AGATCATGAAGGGATTCCTCCAGATGAGCAA |
| | K63I, N25V, | SLDDGRTLSDYN | CGCCTTGCTTTTCAAGGGAAGAGCCTGGATG |
| | Q40E, E51D | LLIDPKKMPLLRL | ACGGTCGCACACTGTCTGACTATAACTTGCTT |
| | | R | ATTGATCCTAAAAAGATGCCACTGCTGCGCTT |
| | | | GCGT |
| CM134 | K6R, T7M, | MHHHHHHGGS | ATGCACCACCACCACCACCACGGTGGATCTG |
| [333; 983] | T12M, T14E, | GMLIFVRMLTGK | GCATGTTGATTTTCGTACGCATGTTGACTGGA |
| | K33H, A46Q, | MIELEVMPSDTIE | AAGATGATCGAGTTGGAAGTGATGCCTTCCG |
| | S65P, L67K, | NVKAKIQDHEGI | ATACTATCGAGAATGTTAAGGCCAAAATCCA |
| | H68M, I61L, | PPDQQRLAFQGT | AGATCATGAAGGGATTCCTCCAGATCAACAA |
| | K63I, E18M, | SLDDGRTLGDYN | CGCCTTGCTTTTCAAGGGACTAGCCTGGATGA |
| | K48T, E51D, | LLIDPKKMPLLRL | CGGTCGCACACTGGGGGACTATAACTTGCTT |
| | S57G | R | ATTGATCCTAAAAAGATGCCACTGCTGCGCTT |
| | | | GCGT |
| CM135 | K6R, T7M, | MHHHHHHGGS | ATGCACCACCACCACCACCACGGTGGATCTG |
| [334; 984] | T12M, T14E, | GMLIFVRMLTGK | GCATGTTGATTTTCGTACGCATGTTGACTGGA |
| | K33H, A46Q, | MIELMVEPSDTIE | AAGATGATCGAGTTGATGGTGGAGCCTTCCG |
| | S65P, L67K, | VVKAKIQDHEGI | ATACTATCGAGGTAGTTAAGGCCAAAATCCA |
| | H68M, I61L, | PPDEQRLAFQGK | AGATCATGAAGGGATTCCTCCAGATGAGCAA |
| | K63I, E16M, | LLEDGRTLSDYNL | CGCCTTGCTTTTCAAGGGAAGTTGCTGGAGG |
| | N25V, Q40E, | LIDPKKMPLLRLR | ACGGTCGCACACTGTCTGACTATAACTTGCTT |
| | S49L | | ATTGATCCTAAAAAGATGCCACTGCTGCGCTT |
| | | | GCGT |
| CM136 | K6R, T7M, | MHHHHHHGGS | ATGCACCACCACCACCACCACGGTGGATCTG |
| [335; 985] | T12M, T14E, | GYLIFVRMLTGK | GCTATTTGATTTTCGTACGCATGTTGACTGGA |
| | K33H, A46Q, | MIELEVMPSDTIE | AAGATGATCGAGTTGGAAGTGATGCCTTCCG |
| | S65P, L67K, | NIKAKIQDHEGIP | ATACTATCGAGAATATTAAGGCCAAAATCCAA |
| | H68M, I61L, | PDQQRLAFQGTS | GATCATGAAGGGATTCCTCCAGATCAACAAC |
| | K63I, E18M, | LDDGRTLGDYNL | GCCTTGCTTTTCAAGGGACTAGCCTGGATGAC |
| | K48T, E51D, | LIDPKKMPLLRM | GGTCGCACACTGGGGGACTATAACTTGCTTA |
| | S57G, M1Y, | R | TTGATCCTAAAAAGATGCCACTGCTGCGCATG |
| | V26I, L73M | | CGT |
| CM137 | K6R, T7M, | MHHHHHHGGS | ATGCACCACCACCACCACCACGGTGGATCTG |
| [336; 986] | T12M, T14E, | GYLIFVRMLTGK | GCTATTTGATTTTCGTACGCATGTTGACTGGA |
| | K33H, A46Q, | MIELEVMPSDTIE | AAGATGATCGAGTTGGAAGTGATGCCTTCCG |
| | S65P, L67K, | NIKAKIQDHEGIP | ATACTATCGAGAATATTAAGGCCAAAATCCAA |
| | H68M, I61L, | PDQQRLAFQGTS | GATCATGAAGGGATTCCTCCAGATCAACAAC |
| | K63I, E18M, | LDDGRTLGDYNL | GCCTTGCTTTTCAAGGGACTAGCCTGGATGAC |
| | K48T, E51D, | LIDPKKMPLLRM | GGTCGCACACTGGGGGACTATAACTTGCTTA |
| | S57G, M1Y, | Q | TTGATCCTAAAAAGATGCCACTGCTGCGCATG |
| | V26I, L73M, | | CAA |
| | R74Q | | |
| CM138 | K6R, T7M, | MHHHHHHGGS | ATGCACCACCACCACCACCACGGTGGATCTG |
| [337; 987] | T12M, T14E, | GYLIFVRMLTGK | GCTATTTGATTTTCGTACGCATGTTGACTGGA |
| | K33H, A46Q, | MIELEVMPSDTIE | AAGATGATCGAGTTGGAAGTGATGCCTTCCG |
| | S65P, L67K, | VIKAKIQDHEGIP | ATACTATCGAGGTAATTAAGGCCAAAATCCA |
| | H68M, I61L, | PDEQRLAFQGTS | AGATCATGAAGGGATTCCTCCAGATGAGCAA |
| | K63I, E18M, | LDDGRTLGDYNL | CGCCTTGCTTTTCAAGGGACTAGCCTGGATGA |
| | K48T, E51D, | LIDPKKMPLLRM | CGGTCGCACACTGGGGGACTATAACTTGCTT |
| | S57G, M1Y, | Q | ATTGATCCTAAAAAGATGCCACTGCTGCGCAT |
| | V26I, L73M, | | GCAA |
| | R74Q, N25V, | | |
| | Q40E, E51D | | |
| CM139 | K6R, T7M, | MHHHHHHGGS | ATGCACCACCACCACCACCACGGTGGATCTG |
| [338; 988] | T12M, T14E, | GMLIFVRMLTGK | GCATGTTGATTTTCGTACGCATGTTGACTGGA |
| | K33H, A46Q, | MIELEVEPSDTIE | AAGATGATCGAGTTGGAAGTGGAGCCTTCCG |
| | S65P, L67K, | NVKAKIQDHEGI | ATACTATCGAGAATGTTAAGGCCAAAATCCA |
| | H68M, I61L, | PPDQQRLTFQGK | AGATCATGAAGGGATTCCTCCAGATCAACAA |
| | K63I, A44T, | LLEDGRTLSDYNL | CGCCTTACTTTTCAAGGGAAGTTGCTGGAGG |
| | S49L | LIDPKKMPLLRLR | ACGGTCGCACACTGTCTGACTATAACTTGCTT |
| | | | ATTGATCCTAAAAAGATGCCACTGCTGCGCTT |
| | | | GCGT |
| CM140 | K6R, T7M, | MHHHHHHGGS | ATGCACCACCACCACCACCACGGTGGATCTG |
| [339; 989] | T12M, T14E, | GMLIFVRMLTGK | GCATGTTGATTTTCGTACGCATGTTGACTGGA |
| | K33H, A46Q, | MIELEVEPSDTIE | AAGATGATCGAGTTGGAAGTGGAGCCTTCCG |
| | S65P, L67K, | NVKAKIQDHEGI | ATACTATCGAGAATGTTAAGGCCAAAATCCA |
| | H68M, I61L, | PLDQQRLTFQGK | AGATCATGAAGGGATTCCTTTGGATCAACAA |
| | K63I, A44T, | LLEDGRTLSDYNL | CGCCTTACTTTTCAAGGGAAGTTGCTGGAGG |
| | S49L, P38L | LIDPKKMPLLRLR | ACGGTCGCACACTGTCTGACTATAACTTGCTT |
| | | | ATTGATCCTAAAAAGATGCCACTGCTGCGCTT |
| | | | GCGT |
| CM141 | K6R, T7M, | MHHHHHHGGS | ATGCACCACCACCACCACCACGGTGGATCTG |
| [340; 990] | T12M, T14E, | GMLIFVRMLTGK | GCATGTTGATTTTCGTACGCATGTTGACTGGA |
| | K33H, A46Q, | MIELEVEPSDTIE | AAGATGATCGAGTTGGAAGTGGAGCCTTCCG |
| | S65P, L67K, | NVKAKIQDHEGI | ATACTATCGAGAATGTTAAGGCCAAAATCCA |
| | H68M, I61L, | PLDQQRLTFQGK | AGATCATGAAGGGATTCCTTTGGATCAACAA |
| | K63I, A44T, | LLEDGRTLSDYNL | CGCCTTACTTTTCAAGGGAAGTTGCTGGAGG |
| | S49L, P38L, | LIDPKKMPLLRLQ | ACGGTCGCACACTGTCTGACTATAACTTGCTT |
| | R74Q | | ATTGATCCTAAAAAGATGCCACTGCTGCGCTT |
| | | | GCAA |
| CM142 | K6R, T7M, | MHHHHHHGGS | ATGCACCACCACCACCACCACGGTGGATCTG |
| [341; 991] | T12M, T14E, | GYLIFVRMLTGK | GCTATTTGATTTTCGTACGCATGTTGACTGGA |
| | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLL | GCCTTACTTTTCAAGGGAAGTTGCTGGAGGA |
| | S49L, P38L, | IDPKKMPLLRMQ | CGGTCGCACACTGTCTGACTATAACTTGCTTA |
| | R74Q, M1Y, | | TTGATCCTAAAAAGATGCCACTGCTGCGCATG |
| | V26I, L73M | | CAA |
| CM143 | K6R, T7M, | MHHHHHHGGS | ATGCACCACCACCACCACCACGGTGGATCTG |
| [342; 992] | T12M, T14E, | GMLIFVRMLTGK | GCATGTTGATTTTCGTACGCATGTTGACTGGA |
| | K33H, A46Q, | MIELEVEPSDTIE | AAGATGATCGAGTTGGAAGTGGAGCCTTCCG |
| | S65P, L67K, | VVKAKIQDHEGI | ATACTATCGAGGTAGTTAAGGCCAAAATCCA |
| | H68M, I61L, | PLDEQRLTFQGK | AGATCATGAAGGGATTCCTTTGGATGAGCAA |
| | K63I, A44T, | LLDDGRTLSDYNL | CGCCTTACTTTTCAAGGGAAGTTGCTGGATGA |
| | S49L, P38L, | LIDPKKMPLLRLQ | CGGTCGCACACTGTCTGACTATAACTTGCTTA |
| | R74Q, N25V, | | TTGATCCTAAAAAGATGCCACTGCTGCGCTTG |
| | Q40E, E51D | | CAA |
| CM144 | K6R, T7M, | MHHHHHHGGS | ATGCACCACCACCACCACCACGGTGGATCTG |
| [343; 993] | T12M, T14E, | GYLIFVRMLTGK | GCTATTTGATTTTCGTACGCATGTTGACTGGA |
| | K33H, A46Q, | MIELEVEPSDTIE | AAGATGATCGAGTTGGAAGTGGAGCCTTCCG |
| | S65P, L67K, | VIKAKIQDHEGIP | ATACTATCGAGGTAATTAAGGCCAAAATCCA |
| | H68M, I61L, | LDEQRLTFQGKLL | AGATCATGAAGGGATTCCTTTGGATGAGCAA |
| | K63I, A44T, | DDGRTLSDYNLLI | CGCCTTACTTTTCAAGGGAAGTTGCTGGATGA |
| | S49L, P38L, | DPKKMPLLRMQ | CGGTCGCACACTGTCTGACTATAACTTGCTTA |
| | R74Q, N25V, | | TTGATCCTAAAAAGATGCCACTGCTGCGCATG |
| | Q40E, E51D, | | CAA |
| | M1Y, V26I, | | |
| | L73M | | |
| CM145 | K6R, T7M, | MHHHHHHGGS | ATGCACCACCACCACCACCACGGTGGATCTG |
| [344; 994] | T12M, T14E, | GMLIFVRMLTGK | GCATGTTGATTTTCGTACGCATGTTGACTGGA |
| | K33H, A46Q, | MIELEVMPSDTIE | AAGATGATCGAGTTGGAAGTGATGCCTTCCG |
| | S65P, L67K, | VVKAKIQDHEGI | ATACTATCGAGGTAGTTAAGGCCAAAATCCA |
| | H68M, I61L, | PPDQQHLTFQGT | AGATCATGAAGGGATTCCTCCAGATCAACAA |
| | K63I, E18M, | LLEDGYTLGDYNL | CATCTTACTTTTCAAGGGACTTTGCTGGAGGA |
| | N25V, R42H, | LIDPKKMPLLRLR | CGGTTATACACTGGGGGACTATAACTTGCTTA |
| | K48T, R54Y, | | TTGATCCTAAAAAGATGCCACTGCTGCGCTTG |
| | S57G, A44T, | | CGT |
| | S49L | | |
| CM146 | K6R, T7M, | MHHHHHHGGS | ATGCACCACCACCACCACCACGGTGGATCTG |
| [345; 995] | T12M, T14E, | GMLIFVRMLTGK | GCATGTTGATTTTCGTACGCATGTTGACTGGA |
| | K33H, A46Q, | MIELEVMPSDTIE | AAGATGATCGAGTTGGAAGTGATGCCTTCCG |
| | S65P, L67K, | VVKAKIQDHEGI | ATACTATCGAGGTAGTTAAGGCCAAAATCCA |
| | H68M, I61L, | PLDQQHLTFQGT | AGATCATGAAGGGATTCCTTTGGATCAACAA |
| | K63I, E18M, | LLEDGYTLGDYNL | CATCTTACTTTTCAAGGGACTTTGCTGGAGGA |
| | N25V, R42H, | LIDPKKMPLLRLR | CGGTTATACACTGGGGGACTATAACTTGCTTA |
| | K48T, R54Y, | | TTGATCCTAAAAAGATGCCACTGCTGCGCTTG |
| | S57G, A44T, | | CGT |
| | S49L, P38L | | |
| CM147 | K6R, T7M, | MHHHHHHGGS | ATGCACCACCACCACCACCACGGTGGATCTG |
| [346; 996] | T12M, T14E, | GMLIFVRMLTGK | GCATGTTGATTTTCGTACGCATGTTGACTGGA |
| | K33H, A46Q, | MIELEVMPSDTIE | AAGATGATCGAGTTGGAAGTGATGCCTTCCG |
| | S65P, L67K, | VVKAKIQDHEGI | ATACTATCGAGGTAGTTAAGGCCAAAATCCA |
| | H68M, I61L, | PLDQQHLTFQGT | AGATCATGAAGGGATTCCTTTGGATCAACAA |
| | K63I, E18M, | LLEDGYTLGDYNL | CATCTTACTTTTCAAGGGACTTTGCTGGAGGA |
| | N25V, R42H, | LIDPKKMPLLRLQ | CGGTTATACACTGGGGGACTATAACTTGCTTA |
| | K48T, R54Y, | | TTGATCCTAAAAAGATGCCACTGCTGCGCTTG |
| | S57G, A44T, | | CAA |
| | S49L, P38L, | | |
| | R74Q | | |
| CM148 | K6R, T7M, | MHHHHHHGGS | ATGCACCACCACCACCACCACGGTGGATCTG |
| [347; 997] | T12M, T14E, | GMLIFVRMLTGK | GCATGTTGATTTTCGTACGCATGTTGACTGGA |
| | K33H, A46Q, | MIELEVMPSDTIE | AAGATGATCGAGTTGGAAGTGATGCCTTCCG |
| | S65P, L67K, | VVKAKIQDHEGI | ATACTATCGAGGTAGTTAAGGCCAAAATCCA |
| | H68M, I61L, | PLDEQHLTFQGT | AGATCATGAAGGGATTCCTTTGGATGAGCAA |
| | K63I, E18M, | LLDDGYTLGDYN | CATCTTACTTTTCAAGGGACTTTGCTGGATGA |
| | N25V, R42H, | LLIDPKKMPLLRL | CGGTTATACACTGGGGGACTATAACTTGCTTA |
| | K48T, R54Y, | Q | TTGATCCTAAAAAGATGCCACTGCTGCGCTTG |
| | S57G, A44T, | | CAA |
| | S49L, P38L, | | |
| | R74Q, N25V, | | |
| | Q40E, E51D | | |
| CM149 | K6R, T7M, | MHHHHHHGGS | ATGCACCACCACCACCACCACGGTGGATCTG |
| [348; 998] | T12M, T14E, | GYLIFVRMLTGK | GCTATTTGATTTTCGTACGCATGTTGACTGGA |
| | K33H, A46Q, | MIELEVMPSDTIE | AAGATGATCGAGTTGGAAGTGATGCCTTCCG |
| | S65P, L67K, | VIKAKIQDHEGIP | ATACTATCGAGGTAATTAAGGCCAAAATCCA |
| | H68M, I61L, | LDEQHLTFQGTL | AGATCATGAAGGGATTCCTTTGGATGAGCAA |
| | K63I, E18M, | LDDGYTLGDYNL | CATCTTACTTTTCAAGGGACTTTGCTGGATGA |
| | N25V, R42H, | LIDPKKMPLLRM | CGGTTATACACTGGGGGACTATAACTTGCTTA |
| | K48T, R54Y, | Q | TTGATCCTAAAAAGATGCCACTGCTGCGCATG |
| | S57G, A44T, | | CAA |
| | S49L, P38L, | | |
| | R74Q, N25V, | | |
| | Q40E, E51D, | | |
| | M1Y, V26I, | | |
| | L73M | | |
| CM199 | K6R, T7M, | MHHHHHHGGS | ATGCACCACCACCACCACCACGGTGGATCTG |
| [349; 999] | T12M, T14E, | GYLIFVRMLTGK | GCTATTTGATTTTCGTACGCATGTTGACTGGA |
| | K33H, A46Q, | MIELEVMPSDTIE | AAGATGATCGAGTTGGAAGTGATGCCTTCCG |
| | S65P, L67K, | VIKAKIQDHEGIP | ATACTATCGAGGTAATTAAGGCCAAAATCCA |
| | H68M, I61L, | PDEQRLAFQGTS | AGATCATGAAGGGATTCCTCCAGATGAGCAA |
| | K63I, E18M, | LDDGRTLGDYNL | CGCCTTGCTTTTCAAGGGACTAGCCTGGATGA |
| | K48T, E51D, | LIDPKKMLVLRM | CGGTCGCACACTGGGGGACTATAACTTGCTT |
| | S57G, M1Y, | Q | ATTGATCCTAAAAAGATGTTGGTACTGCGCAT |
| | V26I, L73M, | | GCAA |
| | R74Q, N25V, | | |
| | Q40E, E51D, | | |
| | P69L, L70V | | |
| CM203 | K6R, T7M, | MHHHHHHGGS | ATGCACCACCACCACCACCACGGTGGATCTG |
| [350; | T12M, T14E, | GYLIFVRMLTGK | GCTATTTGATTTTCGTACGCATGTTGACTGGA |
| 1000] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLL | GCCTTACTTTTCAAGGGAAGTTGCTGGAGGA |
| | S49L, P38L, | IDPKKMLVLRMQ | CGGTCGCACACTGTCTGACTATAACTTGCTTA |
| | R74Q, M1Y, | | TTGATCCTAAAAAGATGTTGGTACTGCGCATG |
| | V26I, L73M, | | CAA |
| | P69L, L70V | | |
| CM204 | K6R, T7M, | MHHHHHHGGS | ATGCACCACCACCACCACCACGGTGGATCTG |
| [351; | T12M, T14E, | GMLIFVRMLTGK | GCATGTTGATTTTCGTACGCATGTTGACTGGA |
| 1001] | K33H, A46Q, | MIELEVEPSDTIE | AAGATGATCGAGTTGGAAGTGGAGCCTTCCG |
| | S65P, L67K, | VVKAKIQDHEGI | ATACTATCGAGGTAGTTAAGGCCAAAATCCA |
| | H68M, I61L, | PLDEQRLTFQGK | AGATCATGAAGGGATTCCTTTGGATGAGCAA |
| | K63I, A44T, | LLDDGRTLSDYNL | CGCCTTACTTTTCAAGGGAAGTTGCTGGATGA |
| | S49L, P38L, | LIDPKKMLVLRLQ | CGGTCGCACACTGTCTGACTATAACTTGCTTA |
| | R74Q, N25V, | | TTGATCCTAAAAAGATGTTGGTACTGCGCTTG |
| | Q40E, E51D, | | CAA |
| | P69L, L70V | | |
| CM208 | K6R, T7M, | MHHHHHHGGS | ATGCACCACCACCACCACCACGGTGGATCTG |
| [352; | T12M, T14E, | GMLIFVRMLTGK | GCATGTTGATTTTCGTACGCATGTTGACTGGA |
| 1002] | K33H, A46Q, | MIELEVMPSDTIE | AAGATGATCGAGTTGGAAGTGATGCCTTCCG |
| | S65P, L67K, | VVKAKIQDHEGI | ATACTATCGAGGTAGTTAAGGCCAAAATCCA |
| | H68M, I61L, | PLDQQHLTFQGT | AGATCATGAAGGGATTCCTTTGGATCAACAA |
| | K63I, E18M, | LLEDGYTLGDYNL | CATCTTACTTTTCAAGGGACTTTGCTGGAGGA |
| | N25V, R42H, | LIDPKKMLVLRLQ | CGGTTATACACTGGGGGACTATAACTTGCTTA |
| | K48T, R54Y, | | TTGATCCTAAAAAGATGTTGGTACTGCGCTTG |
| | S57G, A44T, | | CAA |
| | S49L, P38L, | | |
| | R74Q, P69L, | | |
| | L70V | | |
| CM210 | K6R, T7M, | MHHHHHHGGS | ATGCACCACCACCACCACCACGGTGGATCTG |
| [353; | T12M, T14E, | GYLIFVRMLTGK | GCTATTTGATTTTCGTACGCATGTTGACTGGA |
| 1003] | K33H, A46Q, | MIELEVMPSDTIE | AAGATGATCGAGTTGGAAGTGATGCCTTCCG |
| | S65P, L67K, | VIKAKIQDHEGIP | ATACTATCGAGGTAATTAAGGCCAAAATCCA |
| | H68M, I61L, | LDEQHLTFQGTL | AGATCATGAAGGGATTCCTTTGGATGAGCAA |
| | K63I, E18M, | LDDGYTLGDYNL | CATCTTACTTTTCAAGGGACTTTGCTGGATGA |
| | N25V, R42H, | LIDPKKMLVLRM | CGGTTATACACTGGGGGACTATAACTTGCTTA |
| | K48T, R54Y, | Q | TTGATCCTAAAAAGATGTTGGTACTGCGCATG |
| | S57G, A44T, | | CAA |
| | S49L, P38L, | | |
| | R74Q, N25V, | | |
| | Q40E, E51D, | | |
| | M1Y, V26I, | | |
| | L73M, P69L, | | |
| | L70V | | |
| CM211 | K6R, T7M, | MHHHHHHGGS | ATGCACCACCACCACCACCACGGTGGATCTG |
| [354; | T12M, T14E, | GMLIFVRMLTGK | GCATGTTGATTTTCGTACGCATGTTGACTGGA |
| 1004] | K33H, A46Q, | MIELEVMPSDTIE | AAGATGATCGAGTTGGAAGTGATGCCTTCCG |
| | S65P, L67K, | VVKAKIQDHEGI | ATACTATCGAGGTAGTTAAGGCCAAAATCCA |
| | H68M, I61L, | PPDQQHLAFQG | AGATCATGAAGGGATTCCTCCAGATCAACAA |
| | K63I, E18M, | TSLEDGYTLGDY | CATCTTGCTTTTCAAGGGACTAGCCTGGAGG |
| | N25V, R42H, | NLLIDPKKMLVLR | ACGGTTATACACTGGGGGACTATAACTTGCTT |
| | K48T, R54Y, | LR | ATTGATCCTAAAAAGATGTTGGTACTGCGCTT |
| | S57G, P69L, | | GCGT |
| | L70V | | |
| CM358 | K6R, T7M, | MHHHHHHGGS | ATGCACCACCACCACCACCACGGTGGATCTG |
| [355; | T12M, T14E, | GYLIFVRMLTGK | GCTATTTGATTTTCGTACGCATGTTGACTGGA |
| 1005] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLL | GCCTTACTTTTCAAGGGAAGTTGCTGGAGGA |
| | S49L, P38L, | IDPKKMAVLRM | CGGTCGCACACTGTCTGACTATAACTTGCTTA |
| | R74Q, M1Y, | Q | TTGATCCTAAAAAGATGGCCGTACTGCGCAT |
| | V26I, L73M, | | GCAA |
| | P69A, L70V | | |
| CM359 | K6R, T7M, | MHHHHHHGGS | ATGCACCACCACCACCACCACGGTGGATCTG |
| [356; | T12M, T14E, | GYLIFVRMLTGK | GCTATTTGATTTTCGTACGCATGTTGACTGGA |
| 1006] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLL | GCCTTACTTTTCAAGGGAAGTTGCTGGAGGA |
| | S49L, P38L, | IDPKKMRVLRM | CGGTCGCACACTGTCTGACTATAACTTGCTTA |
| | R74Q, M1Y, | Q | TTGATCCTAAAAAGATGCGCGTACTGCGCAT |
| | V26I, L73M, | | GCAA |
| | P69R, L70V | | |
| CM360 | K6R, T7M, | MHHHHHHGGS | ATGCACCACCACCACCACCACGGTGGATCTG |
| [357; | T12M, T14E, | GYLIFVRMLTGK | GCTATTTGATTTTCGTACGCATGTTGACTGGA |
| 1007] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLL | GCCTTACTTTTCAAGGGAAGTTGCTGGAGGA |
| | S49L, P38L, | IDPKKMNVLRM | CGGTCGCACACTGTCTGACTATAACTTGCTTA |
| | R74Q, M1Y, | Q | TTGATCCTAAAAAGATGAATGTACTGCGCAT |
| | V26I, L73M, | | GCAA |
| | P69N, L70V | | |
| CM361 | K6R, T7M, | MHHHHHHGGS | ATGCACCACCACCACCACCACGGTGGATCTG |
| [358; | T12M, T14E, | GYLIFVRMLTGK | GCTATTTGATTTTCGTACGCATGTTGACTGGA |
| 1008] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLL | GCCTTACTTTTCAAGGGAAGTTGCTGGAGGA |
| | S49L, P38L, | IDPKKMDVLRM | CGGTCGCACACTGTCTGACTATAACTTGCTTA |
| | R74Q, M1Y, | Q | TTGATCCTAAAAAGATGGATGTACTGCGCAT |
| | V26I, L73M, | | GCAA |
| | P69D, L70V | | |
| CM362 | K6R, T7M, | MHHHHHHGGS | ATGCACCACCACCACCACCACGGTGGATCTG |
| [359; | T12M, T14E, | GYLIFVRMLTGK | GCTATTTGATTTTCGTACGCATGTTGACTGGA |
| 1009] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLL | GCCTTACTTTTCAAGGGAAGTTGCTGGAGGA |
| | S49L, P38L, | IDPKKMCVLRM | CGGTCGCACACTGTCTGACTATAACTTGCTTA |
| | R74Q, M1Y, | Q | TTGATCCTAAAAAGATGTGCGTACTGCGCAT |
| | V26I, L73M, | | GCAA |
| | P69C, L70V | | |
| CM363 | K6R, T7M, | MHHHHHHGGS | ATGCACCACCACCACCACCACGGTGGATCTG |
| [360; | T12M, T14E, | GYLIFVRMLTGK | GCTATTTGATTTTCGTACGCATGTTGACTGGA |
| 1010] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLL | GCCTTACTTTTCAAGGGAAGTTGCTGGAGGA |
| | S49L, P38L, | IDPKKMEVLRM | CGGTCGCACACTGTCTGACTATAACTTGCTTA |
| | R74Q, M1Y, | Q | TTGATCCTAAAAAGATGGAGGTACTGCGCAT |
| | V26I, L73M, | | GCAA |
| | P69E, L70V | | |
| CM364 | K6R, T7M, | MHHHHHHGGS | ATGCACCACCACCACCACCACGGTGGATCTG |
| [361; | T12M, T14E, | GYLIFVRMLTGK | GCTATTTGATTTTCGTACGCATGTTGACTGGA |
| 1011] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLL | GCCTTACTTTTCAAGGGAAGTTGCTGGAGGA |
| | S49L, P38L, | IDPKKMQVLRM | CGGTCGCACACTGTCTGACTATAACTTGCTTA |
| | R74Q, M1Y, | Q | TTGATCCTAAAAAGATGCAAGTACTGCGCAT |
| | V26I, L73M, | | GCAA |
| | P69Q, L70V | | |
| CM365 | K6R, T7M, | MHHHHHHGGS | ATGCACCACCACCACCACCACGGTGGATCTG |
| [362; | T12M, T14E, | GYLIFVRMLTGK | GCTATTTGATTTTCGTACGCATGTTGACTGGA |
| 1012] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLL | GCCTTACTTTTCAAGGGAAGTTGCTGGAGGA |
| | S49L, P38L, | IDPKKMGVLRM | CGGTCGCACACTGTCTGACTATAACTTGCTTA |
| | R74Q, M1Y, | Q | TTGATCCTAAAAAGATGGGGGTACTGCGCAT |
| | V26I, L73M, | | GCAA |
| | P69G, L70V | | |
| CM366 | K6R, T7M, | MHHHHHHGGS | ATGCACCACCACCACCACCACGGTGGATCTG |
| [363; | T12M, T14E, | GYLIFVRMLTGK | GCTATTTGATTTTCGTACGCATGTTGACTGGA |
| 1013] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLL | GCCTTACTTTTCAAGGGAAGTTGCTGGAGGA |
| | S49L, P38L, | IDPKKMHVLRM | CGGTCGCACACTGTCTGACTATAACTTGCTTA |
| | R74Q, M1Y, | Q | TTGATCCTAAAAAGATGCATGTACTGCGCATG |
| | V26I, L73M, | | CAA |
| | P69H, L70V | | |
| CM367 | K6R, T7M, | MHHHHHHGGS | ATGCACCACCACCACCACCACGGTGGATCTG |
| [364; | T12M, T14E, | GYLIFVRMLTGK | GCTATTTGATTTTCGTACGCATGTTGACTGGA |
| 1014] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLL | GCCTTACTTTTCAAGGGAAGTTGCTGGAGGA |
| | S49L, P38L, | IDPKKMIVLRMQ | CGGTCGCACACTGTCTGACTATAACTTGCTTA |
| | R74Q, M1Y, | | TTGATCCTAAAAAGATGATTGTACTGCGCATG |
| | V26I, L73M, | | CAA |
| | P69I, L70V | | |
| CM368 | K6R, T7M, | MHHHHHHGGS | ATGCACCACCACCACCACCACGGTGGATCTG |
| [365; | T12M, T14E, | GYLIFVRMLTGK | GCTATTTGATTTTCGTACGCATGTTGACTGGA |
| 1015] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLL | GCCTTACTTTTCAAGGGAAGTTGCTGGAGGA |
| | S49L, P38L, | IDPKKMKVLRM | CGGTCGCACACTGTCTGACTATAACTTGCTTA |
| | R74Q, M1Y, | Q | TTGATCCTAAAAAGATGAAGGTACTGCGCAT |
| | V26I, L73M, | | GCAA |
| | P69K, L70V | | |
| CM369 | K6R, T7M, | MHHHHHHGGS | ATGCACCACCACCACCACCACGGTGGATCTG |
| [366; | T12M, T14E, | GYLIFVRMLTGK | GCTATTTGATTTTCGTACGCATGTTGACTGGA |
| 1016] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLL | GCCTTACTTTTCAAGGGAAGTTGCTGGAGGA |
| | S49L, P38L, | IDPKKMMVLRM | CGGTCGCACACTGTCTGACTATAACTTGCTTA |
| | R74Q, M1Y, | Q | TTGATCCTAAAAAGATGATGGTACTGCGCAT |
| | V26I, L73M, | | GCAA |
| | P69M, L70V | | |
| CM370 | K6R, T7M, | MHHHHHHGGS | ATGCACCACCACCACCACCACGGTGGATCTG |
| [367; | T12M,T14E, | GYLIFVRMLTGK | GCTATTTGATTTTCGTACGCATGTTGACTGGA |
| 1017] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLL | GCCTTACTTTTCAAGGGAAGTTGCTGGAGGA |
| | S49L, P38L, | IDPKKMFVLRM | CGGTCGCACACTGTCTGACTATAACTTGCTTA |
| | R74Q, M1Y, | Q | TTGATCCTAAAAAGATGTTCGTACTGCGCATG |
| | V26I, L73M, | | CAA |
| | P69F, L70V | | |
| CM371 | K6R, T7M, | MHHHHHHGGS | ATGCACCACCACCACCACCACGGTGGATCTG |
| [368; | T12M, T14E, | GYLIFVRMLTGK | GCTATTTGATTTTCGTACGCATGTTGACTGGA |
| 1018] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLL | GCCTTACTTTTCAAGGGAAGTTGCTGGAGGA |
| | S49L, P38L, | IDPKKMSVLRM | CGGTCGCACACTGTCTGACTATAACTTGCTTA |
| | R74Q, M1Y, | Q | TTGATCCTAAAAAGATGTCTGTACTGCGCATG |
| | V26I, L73M, | | CAA |
| | P69S, L70V | | |
| CM372 | K6R, T7M, | MHHHHHHGGS | ATGCACCACCACCACCACCACGGTGGATCTG |
| [369; | T12M, T14E, | GYLIFVRMLTGK | GCTATTTGATTTTCGTACGCATGTTGACTGGA |
| 1019] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLL | GCCTTACTTTTCAAGGGAAGTTGCTGGAGGA |
| | S49L, P38L, | IDPKKMTVLRM | CGGTCGCACACTGTCTGACTATAACTTGCTTA |
| | R74Q, M1Y, | Q | TTGATCCTAAAAAGATGACTGTACTGCGCATG |
| | V26I, L73M, | | CAA |
| | P69T, L70V | | |
| CM373 | K6R, T7M, | MHHHHHHGGS | ATGCACCACCACCACCACCACGGTGGATCTG |
| [370; | T12M, T14E, | GYLIFVRMLTGK | GCTATTTGATTTTCGTACGCATGTTGACTGGA |
| 1020] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLL | GCCTTACTTTTCAAGGGAAGTTGCTGGAGGA |
| | S49L, P38L, | IDPKKMWVLRM | CGGTCGCACACTGTCTGACTATAACTTGCTTA |
| | R74Q, M1Y, | Q | TTGATCCTAAAAAGATGTGGGTACTGCGCAT |
| | V26I, L73M, | | GCAA |
| | P69W, L70V | | |
| CM374 | K6R, T7M, | MHHHHHHGGS | ATGCACCACCACCACCACCACGGTGGATCTG |
| [371; | T12M, T14E, | GYLIFVRMLTGK | GCTATTTGATTTTCGTACGCATGTTGACTGGA |
| 1021] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLL | GCCTTACTTTTCAAGGGAAGTTGCTGGAGGA |
| | S49L, P38L, | IDPKKMYVLRM | CGGTCGCACACTGTCTGACTATAACTTGCTTA |
| | R74Q, M1Y, | Q | TTGATCCTAAAAAGATGTATGTACTGCGCATG |
| | V26I, L73M, | | CAA |
| | P69Y, L70V | | |
| CM375 | K6R, T7M, | MHHHHHHGGS | ATGCACCACCACCACCACCACGGTGGATCTG |
| [372; | T12M, T14E, | GYLIFVRMLTGK | GCTATTTGATTTTCGTACGCATGTTGACTGGA |
| 1022] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLL | GCCTTACTTTTCAAGGGAAGTTGCTGGAGGA |
| | S49L, P38L, | IDPKKMVVLRM | CGGTCGCACACTGTCTGACTATAACTTGCTTA |
| | R74Q, M1Y, | Q | TTGATCCTAAAAAGATGGTAGTACTGCGCAT |
| | V26I, L73M, | | GCAA |
| | P69V, L70V | | |
| CM376 | K6R, T7M, | MHHHHHHGGS | ATGCACCACCACCACCACCACGGTGGATCTG |
| [373; | T12M, T14E, | GYLIFVRMLTGK | GCTATTTGATTTTCGTACGCATGTTGACTGGA |
| 1023] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLL | GCCTTACTTTTCAAGGGAAGTTGCTGGAGGA |
| | S49L, P38L, | IDPKKMLALRMQ | CGGTCGCACACTGTCTGACTATAACTTGCTTA |
| | R74Q, M1Y, | | TTGATCCTAAAAAGATGTTGGCCCTGCGCATG |
| | V26I, L73M, | | CAA |
| | P69L, L70A | | |
| CM377 | K6R, T7M, | MHHHHHHGGS | ATGCACCACCACCACCACCACGGTGGATCTG |
| [374; | T12M, T14E, | GYLIFVRMLTGK | GCTATTTGATTTTCGTACGCATGTTGACTGGA |
| 1024] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLL | GCCTTACTTTTCAAGGGAAGTTGCTGGAGGA |
| | S49L, P38L, | IDPKKMLRLRMQ | CGGTCGCACACTGTCTGACTATAACTTGCTTA |
| | R74Q, M1Y, | | TTGATCCTAAAAAGATGTTGCGCCTGCGCATG |
| | V26I, L73M, | | CAA |
| | P69L, L70R | | |
| CM378 | K6R, T7M, | MHHHHHHGGS | ATGCACCACCACCACCACCACGGTGGATCTG |
| [375; | T12M, T14E, | GYLIFVRMLTGK | GCTATTTGATTTTCGTACGCATGTTGACTGGA |
| 1025] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLL | GCCTTACTTTTCAAGGGAAGTTGCTGGAGGA |
| | S49L, P38L, | IDPKKMLNLRM | CGGTCGCACACTGTCTGACTATAACTTGCTTA |
| | R74Q, M1Y, | Q | TTGATCCTAAAAAGATGTTGAATCTGCGCATG |
| | V26I, L73M, | | CAA |
| | P69L, L70N | | |
| CM379 | K6R, T7M, | MHHHHHHGGS | ATGCACCACCACCACCACCACGGTGGATCTG |
| [376; | T12M, T14E, | GYLIFVRMLTGK | GCTATTTGATTTTCGTACGCATGTTGACTGGA |
| 1026] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLL | GCCTTACTTTTCAAGGGAAGTTGCTGGAGGA |
| | S49L, P38L, | IDPKKMLDLRM | CGGTCGCACACTGTCTGACTATAACTTGCTTA |
| | R74Q, M1Y, | Q | TTGATCCTAAAAAGATGTTGGATCTGCGCATG |
| | V26I, L73M, | | CAA |
| | P69L, L70D | | |
| CM380 | K6R, T7M, | MHHHHHHGGS | ATGCACCACCACCACCACCACGGTGGATCTG |
| [377; | T12M, T14E, | GYLIFVRMLTGK | GCTATTTGATTTTCGTACGCATGTTGACTGGA |
| 1027] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLL | GCCTTACTTTTCAAGGGAAGTTGCTGGAGGA |
| | S49L, P38L, | IDPKKMLCLRMQ | CGGTCGCACACTGTCTGACTATAACTTGCTTA |
| | R74Q, M1Y, | | TTGATCCTAAAAAGATGTTGTGCCTGCGCATG |
| | V26I, L73M, | | CAA |
| | P69L, L70C | | |
| CM381 | K6R, T7M, | MHHHHHHGGS | ATGCACCACCACCACCACCACGGTGGATCTG |
| [378; | T12M, T14E, | GYLIFVRMLTGK | GCTATTTGATTTTCGTACGCATGTTGACTGGA |
| 1028] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLL | GCCTTACTTTTCAAGGGAAGTTGCTGGAGGA |
| | S49L, P38L, | IDPKKMLELRMQ | CGGTCGCACACTGTCTGACTATAACTTGCTTA |
| | R74Q, M1Y, | | TTGATCCTAAAAAGATGTTGGAGCTGCGCAT |
| | V26I, L73M, | | GCAA |
| | P69L, L70E | | |
| CM382 | K6R, T7M, | MHHHHHHGGS | ATGCACCACCACCACCACCACGGTGGATCTG |
| [379; | T12M, T14E, | GYLIFVRMLTGK | GCTATTTGATTTTCGTACGCATGTTGACTGGA |
| 1029] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLL | GCCTTACTTTTCAAGGGAAGTTGCTGGAGGA |
| | S49L, P38L, | IDPKKMLQLRM | CGGTCGCACACTGTCTGACTATAACTTGCTTA |
| | R74Q, M1Y, | Q | TTGATCCTAAAAAGATGTTGCAACTGCGCATG |
| | V26I, L73M, | | CAA |
| | P69L, L700 | | |
| CM383 | K6R, T7M, | MHHHHHHGGS | ATGCACCACCACCACCACCACGGTGGATCTG |
| [380; | T12M, T14E, | GYLIFVRMLTGK | GCTATTTGATTTTCGTACGCATGTTGACTGGA |
| 1030] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLL | GCCTTACTTTTCAAGGGAAGTTGCTGGAGGA |
| | S49L, P38L, | IDPKKMLGLRM | CGGTCGCACACTGTCTGACTATAACTTGCTTA |
| | R74Q, M1Y, | Q | TTGATCCTAAAAAGATGTTGGGGCTGCGCAT |
| | V26I, L73M, | | GCAA |
| | P69L, L70G | | |
| CM384 | K6R, T7M, | MHHHHHHGGS | ATGCACCACCACCACCACCACGGTGGATCTG |
| [381; | T12M, T14E, | GYLIFVRMLTGK | GCTATTTGATTTTCGTACGCATGTTGACTGGA |
| 1031] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLL | GCCTTACTTTTCAAGGGAAGTTGCTGGAGGA |
| | S49L, P38L, | IDPKKMLHLRM | CGGTCGCACACTGTCTGACTATAACTTGCTTA |
| | R74Q, M1Y, | Q | TTGATCCTAAAAAGATGTTGCATCTGCGCATG |
| | V26I, L73M, | | CAA |
| | P69L, L70H | | |
| CM385 | K6R, T7M, | MHHHHHHGGS | ATGCACCACCACCACCACCACGGTGGATCTG |
| [382; | T12M, T14E, | GYLIFVRMLTGK | GCTATTTGATTTTCGTACGCATGTTGACTGGA |
| 1032] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLL | GCCTTACTTTTCAAGGGAAGTTGCTGGAGGA |
| | S49L, P38L, | IDPKKMLILRMQ | CGGTCGCACACTGTCTGACTATAACTTGCTTA |
| | R74Q, M1Y, | | TTGATCCTAAAAAGATGTTGATTCTGCGCATG |
| | V26I, L73M, | | CAA |
| | P69L, L70I | | |
| CM386 | K6R, T7M, | MHHHHHHGGS | ATGCACCACCACCACCACCACGGTGGATCTG |
| [383; | T12M, T14E, | GYLIFVRMLTGK | GCTATTTGATTTTCGTACGCATGTTGACTGGA |
| 1033] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLL | GCCTTACTTTTCAAGGGAAGTTGCTGGAGGA |
| | S49L, P38L, | IDPKKMLKLRMQ | CGGTCGCACACTGTCTGACTATAACTTGCTTA |
| | R74Q, M1Y, | | TTGATCCTAAAAAGATGTTGAAGCTGCGCAT |
| | V26I, L73M, | | GCAA |
| | P69L, L70K | | |
| CM387 | K6R, T7M, | MHHHHHHGGS | ATGCACCACCACCACCACCACGGTGGATCTG |
| [384; | T12M, T14E, | GYLIFVRMLTGK | GCTATTTGATTTTCGTACGCATGTTGACTGGA |
| 1034] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLL | GCCTTACTTTTCAAGGGAAGTTGCTGGAGGA |
| | S49L, P38L, | IDPKKMLMLRM | CGGTCGCACACTGTCTGACTATAACTTGCTTA |
| | R74Q, M1Y, | Q | TTGATCCTAAAAAGATGTTGATGCTGCGCATG |
| | V26I, L73M, | | CAA |
| | P69L, L70M | | |
| CM388 | K6R, T7M, | MHHHHHHGGS | ATGCACCACCACCACCACCACGGTGGATCTG |
| [385; | T12M, T14E, | GYLIFVRMLTGK | GCTATTTGATTTTCGTACGCATGTTGACTGGA |
| 1035] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLL | GCCTTACTTTTCAAGGGAAGTTGCTGGAGGA |
| | S49L, P38L, | IDPKKMLFLRMQ | CGGTCGCACACTGTCTGACTATAACTTGCTTA |
| | R74Q, M1Y, | | TTGATCCTAAAAAGATGTTGTTCCTGCGCATG |
| | V26I, L73M, | | CAA |
| | P69L, L70F | | |
| CM389 | K6R, T7M, | MHHHHHHGGS | ATGCACCACCACCACCACCACGGTGGATCTG |
| [386; | T12M, T14E, | GYLIFVRMLTGK | GCTATTTGATTTTCGTACGCATGTTGACTGGA |
| 1036] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLL | GCCTTACTTTTCAAGGGAAGTTGCTGGAGGA |
| | S49L, P38L, | IDPKKMLPLRMQ | CGGTCGCACACTGTCTGACTATAACTTGCTTA |
| | R74Q, M1Y, | | TTGATCCTAAAAAGATGTTGCCTCTGCGCATG |
| | V26I, L73M, | | CAA |
| | P69L, L70P | | |
| CM390 | K6R, T7M, | MHHHHHHGGS | ATGCACCACCACCACCACCACGGTGGATCTG |
| [387; | T12M, T14E, | GYLIFVRMLTGK | GCTATTTGATTTTCGTACGCATGTTGACTGGA |
| 1037] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLL | GCCTTACTTTTCAAGGGAAGTTGCTGGAGGA |
| | S49L, P38L, | IDPKKMLSLRMQ | CGGTCGCACACTGTCTGACTATAACTTGCTTA |
| | R74Q, M1Y, | | TTGATCCTAAAAAGATGTTGTCTCTGCGCATG |
| | V26I, L73M, | | CAA |
| | P69L, L70S | | |
| CM391 | K6R, T7M, | MHHHHHHGGS | ATGCACCACCACCACCACCACGGTGGATCTG |
| [388; | T12M, T14E, | GYLIFVRMLTGK | GCTATTTGATTTTCGTACGCATGTTGACTGGA |
| 1038] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLL | GCCTTACTTTTCAAGGGAAGTTGCTGGAGGA |
| | S49L, P38L, | IDPKKMLTLRMQ | CGGTCGCACACTGTCTGACTATAACTTGCTTA |
| | R74Q, M1Y, | | TTGATCCTAAAAAGATGTTGACTCTGCGCATG |
| | V26I, L73M, | | CAA |
| | P69L, L70T | | |
| CM392 | K6R, T7M, | MHHHHHHGGS | ATGCACCACCACCACCACCACGGTGGATCTG |
| [389; | T12M, T14E, | GYLIFVRMLTGK | GCTATTTGATTTTCGTACGCATGTTGACTGGA |
| 1039] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLL | GCCTTACTTTTCAAGGGAAGTTGCTGGAGGA |
| | S49L, P38L, | IDPKKMLWLRM | CGGTCGCACACTGTCTGACTATAACTTGCTTA |
| | R74Q, M1Y, | Q | TTGATCCTAAAAAGATGTTGTGGCTGCGCAT |
| | V26I, L73M, | | GCAA |
| | P69L, L70W | | |
| CM393 | K6R, T7M, | MHHHHHHGGS | ATGCACCACCACCACCACCACGGTGGATCTG |
| [390; | T12M, T14E, | GYLIFVRMLTGK | GCTATTTGATTTTCGTACGCATGTTGACTGGA |
| 1040] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLL | GCCTTACTTTTCAAGGGAAGTTGCTGGAGGA |
| | S49L, P38L, | IDPKKMLYLRMQ | CGGTCGCACACTGTCTGACTATAACTTGCTTA |
| | R74Q, M1Y, | | TTGATCCTAAAAAGATGTTGTATCTGCGCATG |
| | V26I, L73M, | | CAA |
| | P69L, L70Y | | |
| CM429 | K6R, T7M, | MHHHHHHGGS | ATGCACCACCACCACCACCACGGTGGATCTG |
| [391; | T12M, T14E, | GYMIFVRMLTGK | GCTATATGATTTTCGTACGCATGTTGACTGGA |
| 1041] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLP | GCCTTACTTTTCAAGGGAAGTTGCTGGAGGA |
| | S49L, P38L, | ISPEKMAVLRMQ | CGGTCGCACACTGTCTGACTATAACTTGCCTA |
| | R74Q, M1Y, | | TTTCTCCTGAGAAGATGGCCGTACTGCGCATG |
| | V26I, L73M, | | CAA |
| | P69A, L70V, | | |
| | L2M, L62P, | | |
| | D64S, K66E | | |
| CM430 | K6R, T7M, | MHHHHHHGGS | ATGCACCACCACCACCACCACGGTGGATCTG |
| [392; | T12M, T14E, | GYMIFVRMLTGK | GCTATATGATTTTCGTACGCATGTTGACTGGA |
| 1042] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLP | GCTTACTTTTCAAGGGAAGTTGCTGGAGGA |
| | S49L, P38L, | ISPEKMGVLRMQ | CGGTCGCACACTGTCTGACTATAACTTGCCTA |
| | R74Q, M1Y, | | TTTCTCCTGAGAAGATGGGGGTACTGCGCAT |
| | V26I, L73M, | | GCAA |
| | P69G, L70V, | | |
| | L2M, L62P, | | |
| | D64S, K66E | | |
| CM431 | K6R, T7M, | MHHHHHHGGS | ATGCACCACCACCACCACCACGGTGGATCTG |
| [393; | T12M, T14E, | GYMIFVRMLTGK | GCTATATGATTTTCGTACGCATGTTGACTGGA |
| 1043] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLP | GCCTTACTTTTCAAGGGAAGTTGCTGGAGGA |
| | S49L, P38L, | ISPEKMLMLRM | CGGTCGCACACTGTCTGACTATAACTTGCCTA |
| | R74Q, M1Y, | Q | TTTCTCCTGAGAAGATGTTGATGCTGCGCATG |
| | V26I, L73M, | | CAA |
| | P69L, L70M, | | |
| | L2M, L62P, | | |
| | D64S, K66E | | |
| CM432 | K6R, T7M, | MHHHHHHGGS | ATGCACCACCACCACCACCACGGTGGATCTG |
| [394; | T12M, T14E, | GYMIFVRMLTGK | GCTATATGATTTTCGTACGCATGTTGACTGGA |
| 1044] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLP | GCCTTACTTTTCAAGGGAAGTTGCTGGAGGA |
| | S49L, P38L, | ISPEKMAMLRM | CGGTCGCACACTGTCTGACTATAACTTGCCTA |
| | R74Q, M1Y, | Q | TTTCTCCTGAGAAGATGGCCATGCTGCGCATG |
| | V26I, L73M, | | CAA |
| | P69A, L70M, | | |
| | L2M, L62P, | | |
| | D64S, K66E | | |
| CM433 | K6R, T7M, | MHHHHHHGGS | ATGCACCACCACCACCACCACGGTGGATCTG |
| [395; | T12M, T14E, | GYMIFVRMLTGK | GCTATATGATTTTCGTACGCATGTTGACTGGA |
| 1045] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLP | GCCTTACTTTTCAAGGGAAGTTGCTGGAGGA |
| | S49L, P38L, | ISPEKMAFLRMQ | CGGTCGCACACTGTCTGACTATAACTTGCCTA |
| | R74Q, M1Y, | | TTTCTCCTGAGAAGATGGCCTTCCTGCGCATG |
| | V26I, L73M, | | CAA |
| | P69A, L70F, | | |
| | L2M, L62P, | | |
| | D64S, K66E | | |
| CM434 | K6R, T7M, | MHHHHHHGGS | ATGCACCACCACCACCACCACGGTGGATCTG |
| [396; | T12M, T14E, | GYMIFVRMLTGK | GCTATATGATTTTCGTACGCATGTTGACTGGA |
| 1046] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLP | GCCTTACTTTTCAAGGGAAGTTGCTGGAGGA |
| | S49L, P38L, | ISPEKMACLRMQ | CGGTCGCACACTGTCTGACTATAACTTGCCTA |
| | R74Q, M1Y, | | TTTCTCCTGAGAAGATGGCCTGCCTGCGCATG |
| | V26I, L73M, | | CAA |
| | P69A, L70C, | | |
| | L2M, L62P, | | |
| | D64S, K66E | | |
| CM435 | K6R, T7M, | MHHHHHHGGS | ATGCACCACCACCACCACCACGGTGGATCTG |
| [397; | T12M, T14E, | GYMIFVRMLTGK | GCTATATGATTTTCGTACGCATGTTGACTGGA |
| 1047] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLP | GCCTTACTTTTCAAGGGAAGTTGCTGGAGGA |
| | S49L, P38L, | ISPEKMGMLRM | CGGTCGCACACTGTCTGACTATAACTTGCCTA |
| | R74Q, M1Y, | Q | TTTCTCCTGAGAAGATGGGGATGCTGCGCAT |
| | V26I, L73M, | | GCAA |
| | P69G, L70M, | | |
| | L2M, L62P, | | |
| | D64S, K66E | | |
| CM436 | K6R, T7M, | MHHHHHHGGS | ATGCACCACCACCACCACCACGGTGGATCTG |
| [398; | T12M, T14E, | GYMIFVRMLTGK | GCTATATGATTTTCGTACGCATGTTGACTGGA |
| 1048] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLP | GCCTTACTTTTCAAGGGAAGTTGCTGGAGGA |
| | S49L, P38L, | ISPEKMGFLRMQ | CGGTCGCACACTGTCTGACTATAACTTGCCTA |
| | R74Q, M1Y, | | TTTCTCCTGAGAAGATGGGGTTCCTGCGCATG |
| | V26I, L73M, | | CAA |
| | P69G, L70F, | | |
| | L2M, L62P, | | |
| | D64S, K66E | | |
| CM437 | K6R, T7M, | MHHHHHHGGS | ATGCACCACCACCACCACCACGGTGGATCTG |
| [399; | T12M, T14E, | GYMIFVRMLTGK | GCTATATGATTTTCGTACGCATGTTGACTGGA |
| 1049] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLP | GCCTTACTTTTCAAGGGAAGTTGCTGGAGGA |
| | S49L, P38L, | ISPEKMGCLRMQ | CGGTCGCACACTGTCTGACTATAACTTGCCTA |
| | R74Q, M1Y, | | TTTCTCCTGAGAAGATGGGGTGCCTGCGCAT |
| | V26I, L73M, | | GCAA |
| | P69G, L70C, | | |
| | L2M, L62P, | | |
| | D64S, K66E | | |
| CM438 | K6R, T7M, | MHHHHHHGGS | ATGCACCACCACCACCACCACGGTGGATCTG |
| [400; | T12M, T14E, | GYMIFVRMLTGK | GCTATATGATTTTCGTACGCATGTTGACTGGA |
| 1050] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLP | GCCTTACTTTTCAAGGGAAGTTGCTGGAGGA |
| | S49L, P38L, | ISPEKMCMLRM | CGGTCGCACACTGTCTGACTATAACTTGCCTA |
| | R74Q, M1Y, | Q | TTTCTCCTGAGAAGATGTGCATGCTGCGCATG |
| | V26I, L73M, | | CAA |
| | P69C, L70M, | | |
| | L2M, L62P, | | |
| | D64S, K66E | | |
| CM439 | K6R, T7M, | MHHHHHHGGS | ATGCACCACCACCACCACCACGGTGGATCTG |
| [401; | T12M, T14E, | GYMIFVRMLTGK | GCTATATGATTTTCGTACGCATGTTGACTGGA |
| 1051] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLP | GCCTTACTTTTCAAGGGAAGTTGCTGGAGGA |
| | S49L, P38L, | ISPEKMMMLRM | CGGTCGCACACTGTCTGACTATAACTTGCCTA |
| | R74Q, M1Y, | Q | TTTCTCCTGAGAAGATGATGATGCTGCGCATG |
| | V26I, L73M, | | CAA |
| | P69M, L70M, | | |
| | L2M, L62P, | | |
| | D64S, K66E | | |
| CM440 | K6R, T7M, | MHHHHHHGGS | ATGCACCACCACCACCACCACGGTGGATCTG |
| [402; | T12M, T14E, | GYMIFVRMLTGK | GCTATATGATTTTCGTACGCATGTTGACTGGA |
| 1052] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLP | GCCTTACTTTTCAAGGGAAGTTGCTGGAGGA |
| | S49L, P38L, | ISPEKMFMLRM | CGGTCGCACACTGTCTGACTATAACTTGCCTA |
| | R74Q, M1Y, | Q | TTTCTCCTGAGAAGATGTTCATGCTGCGCATG |
| | V26I, L73M, | | CAA |
| | P69F, L70M, | | |
| | L2M, L62P, | | |
| | D64S, K66E | | |
| CM441 | K6R, T7M, | MHHHHHHGGS | ATGCACCACCACCACCACCACGGTGGATCTG |
| [403; | T12M, T14E, | GYMIFVRMLTGK | GCTATATGATTTTCGTACGCATGTTGACTGGA |
| 1053] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNL | GCCTTACTTTTCAAGGGAAGTTGCTGGAGGA |
| | S49L, P38L, | AISPEKMAVLRM | CGGTCGCACACTGTCTGACTATAACTTGGCCA |
| | R74Q, M1Y, | Q | TTTCTCCTGAGAAGATGGCCGTACTGCGCATG |
| | V26I, L73M, | | CAA |
| | P69A, L70V, | | |
| | L2M, L62A, | | |
| | D64S, K66E | | |
| CM442 | K6R, T7M, | MHHHHHHGGS | ATGCACCACCACCACCACCACGGTGGATCTG |
| [404; | T12M, T14E, | GYMIFVRMLTGK | GCTATATGATTTTCGTACGCATGTTGACTGGA |
| 1054] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLR | GCCTTACTTTTCAAGGGAAGTTGCTGGAGGA |
| | S49L, P38L, | ISPEKMAVLRMQ | CGGTCGCACACTGTCTGACTATAACTTGCGCA |
| | R74Q, M1Y, | | TTTCTCCTGAGAAGATGGCCGTACTGCGCATG |
| | V26I, L73M, | | CAA |
| | P69A, L70V, | | |
| | L2M, L62R, | | |
| | D64S, K66E | | |
| CM443 | K6R, T7M, | MHHHHHHGGS | ATGCACCACCACCACCACCACGGTGGATCTG |
| [405; | T12M, T14E, | GYMIFVRMLTGK | GCTATATGATTTTCGTACGCATGTTGACTGGA |
| 1055] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNL | GCCTTACTTTTCAAGGGAAGTTGCTGGAGGA |
| | S49L, P38L, | NISPEKMAVLRM | CGGTCGCACACTGTCTGACTATAACTTGAATA |
| | R74Q, M1Y, | Q | TTTCTCCTGAGAAGATGGCCGTACTGCGCATG |
| | V26I, L73M, | | CAA |
| | P69A, L70V, | | |
| | L2M, L62N, | | |
| | D64S, K66E | | |
| CM444 | K6R, T7M, | MHHHHHHGGS | ATGCACCACCACCACCACCACGGTGGATCTG |
| [406; | T12M, T14E, | GYMIFVRMLTGK | GCTATATGATTTTCGTACGCATGTTGACTGGA |
| 1056] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNL | GCCTTACTTTTCAAGGGAAGTTGCTGGAGGA |
| | S49L, P38L, | DISPEKMAVLRM | CGGTCGCACACTGTCTGACTATAACTTGGATA |
| | R74Q, M1Y, | Q | TTTCTCCTGAGAAGATGGCCGTACTGCGCATG |
| | V26I, L73M, | | CAA |
| | P69A, L70V, | | |
| | L2M, L62D, | | |
| | D64S, K66E | | |
| CM445 | K6R, T7M, | MHHHHHHGGS | ATGCACCACCACCACCACCACGGTGGATCTG |
| [407; | T12M, T14E, | GYMIFVRMLTGK | GCTATATGATTTTCGTACGCATGTTGACTGGA |
| 1057] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLC | GCCTTACTTTTCAAGGGAAGTTGCTGGAGGA |
| | S49L, P38L, | ISPEKMAVLRMQ | CGGTCGCACACTGTCTGACTATAACTTGTGCA |
| | R74Q, M1Y, | | TTTCTCCTGAGAAGATGGCCGTACTGCGCATG |
| | V26I, L73M, | | CAA |
| | P69A, L70V, | | |
| | L2M, L62C, | | |
| | D64S, K66E | | |
| CM446 | K6R, T7M, | MHHHHHHGGS | ATGCACCACCACCACCACCACGGTGGATCTG |
| [408; | T12M, T14E, | GYMIFVRMLTGK | GCTATATGATTTTCGTACGCATGTTGACTGGA |
| 1058] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLE | GCCTTACTTTTCAAGGGAAGTTGCTGGAGGA |
| | S49L, P38L, | ISPEKMAVLRMQ | CGGTCGCACACTGTCTGACTATAACTTGGAG |
| | R74Q, M1Y, | | ATTTCTCCTGAGAAGATGGCCGTACTGCGCAT |
| | V26I, L73M, | | GCAA |
| | P69A, L70V, | | |
| | L2M, L62E, | | |
| | D64S, K66E | | |
| CM447 | K6R, T7M, | MHHHHHHGGS | ATGCACCACCACCACCACCACGGTGGATCTG |
| [409; | T12M, T14E, | GYMIFVRMLTGK | GCTATATGATTTTCGTACGCATGTTGACTGGA |
| 1059] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNL | GCCTTACTTTTCAAGGGAAGTTGCTGGAGGA |
| | S49L, P38L, | QISPEKMAVLRM | CGGTCGCACACTGTCTGACTATAACTTGCAAA |
| | R74Q, M1Y, | Q | TTTCTCCTGAGAAGATGGCCGTACTGCGCATG |
| | V26I, L73M, | | CAA |
| | P69A, L70V, | | |
| | L2M, L620, | | |
| | D64S, K66E | | |
| CM448 | K6R, T7M, | MHHHHHHGGS | ATGCACCACCACCACCACCACGGTGGATCTG |
| [410; | T12M, T14E, | GYMIFVRMLTGK | GCTATATGATTTTCGTACGCATGTTGACTGGA |
| 1060] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNL | GCCTTACTTTTCAAGGGAAGTTGCTGGAGGA |
| | S49L, P38L, | GISPEKMAVLRM | CGGTCGCACACTGTCTGACTATAACTTGGGG |
| | R74Q, M1Y, | Q | ATTTCTCCTGAGAAGATGGCCGTACTGCGCAT |
| | V26I, L73M, | | GCAA |
| | P69A, L70V, | | |
| | L2M, L62G, | | |
| | D64S, K66E | | |
| CM449 | K6R, T7M, | MHHHHHHGGS | ATGCACCACCACCACCACCACGGTGGATCTG |
| [411; | T12M, T14E, | GYMIFVRMLTGK | GCTATATGATTTTCGTACGCATGTTGACTGGA |
| 1061] | K33H, A46Q., | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNL | GCCTTACTTTTCAAGGGAAGTTGCTGGAGGA |
| | S49L, P38L, | HISPEKMAVLRM | CGGTCGCACACTGTCTGACTATAACTTGCATA |
| | R74Q, M1Y, | Q | TTTCTCCTGAGAAGATGGCCGTACTGCGCATG |
| | V26I, L73M, | | CAA |
| | P69A, L70V, | | |
| | L2M, L62H, | | |
| | D64S, K66E | | |
| CM450 | K6R, T7M, | MHHHHHHGGS | ATGCACCACCACCACCACCACGGTGGATCTG |
| [412; | T12M, T14E, | GYMIFVRMLTGK | GCTATATGATTTTCGTACGCATGTTGACTGGA |
| 1062] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLII | GCCTTACTTTTCAAGGGAAGTTGCTGGAGGA |
| | S49L, P38L, | SPEKMAVLRMQ | CGGTCGCACACTGTCTGACTATAACTTGATTA |
| | R74Q, M1Y, | | TTTCTCCTGAGAAGATGGCCGTACTGCGCATG |
| | V26I, L73M, | | CAA |
| | P69A, L70V, | | |
| | L2M, L62I, | | |
| | D64S, K66E | | |
| CM451 | K6R, T7M, | MHHHHHHGGS | ATGCACCACCACCACCACCACGGTGGATCTG |
| [413; | T12M, T14E, | GYMIFVRMLTGK | GCTATATGATTTTCGTACGCATGTTGACTGGA |
| 1063] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLK | GCCTTACTTTTCAAGGGAAGTTGCTGGAGGA |
| | S49L, P38L, | ISPEKMAVLRMQ | CGGTCGCACACTGTCTGACTATAACTTGAAGA |
| | R74Q, M1Y, | | TTTCTCCTGAGAAGATGGCCGTACTGCGCATG |
| | V26I, L73M, | | CAA |
| | P69A, L70V, | | |
| | L2M, L62K, | | |
| | D64S, K66E | | |
| CM452 | K6R, T7M, | MHHHHHHGGS | ATGCACCACCACCACCACCACGGTGGATCTG |
| [414; | T12M, T14E, | GYMIFVRMLTGK | GCTATATGATTTTCGTACGCATGTTGACTGGA |
| 1064] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNL | GCCTTACTTTTCAAGGGAAGTTGCTGGAGGA |
| | S49L, P38L, | MISPEKMAVLR | CGGTCGCACACTGTCTGACTATAACTTGATGA |
| | R74Q, M1Y, | MQ | TTTCTCCTGAGAAGATGGCCGTACTGCGCATG |
| | V26I, L73M, | | CAA |
| | P69A, L70V, | | |
| | L2M, L62M, | | |
| | D64S, K66E | | |
| CM453 | K6R, T7M, | MHHHHHHGGS | ATGCACCACCACCACCACCACGGTGGATCTG |
| [415; | T12M, T14E, | GYMIFVRMLTGK | GCTATATGATTTTCGTACGCATGTTGACTGGA |
| 1065] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLF | GCCTTACTTTTCAAGGGAAGTTGCTGGAGGA |
| | S49L, P38L, | ISPEKMAVLRMQ | CGGTCGCACACTGTCTGACTATAACTTGTTCA |
| | R74Q, M1Y, | | TTTCTCCTGAGAAGATGGCCGTACTGCGCATG |
| | V26I, L73M, | | CAA |
| | P69A, L70V, | | |
| | L2M, L62F, | | |
| | D64S, K66E | | |
| CM454 | K6R, T7M, | MHHHHHHGGS | ATGCACCACCACCACCACCACGGTGGATCTG |
| [416; | T12M, T14E, | GYMIFVRMLTGK | GCTATATGATTTTCGTACGCATGTTGACTGGA |
| 1066] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLS | GCCTTACTTTTCAAGGGAAGTTGCTGGAGGA |
| | S49L, P38L, | ISPEKMAVLRMQ | CGGTCGCACACTGTCTGACTATAACTTGTCTA |
| | R74Q, M1Y, | | TTTCTCCTGAGAAGATGGCCGTACTGCGCATG |
| | V26I, L73M, | | CAA |
| | P69A, L70V, | | |
| | L2M, L62S, | | |
| | D64S, K66E | | |
| CM455 | K6R, T7M, | MHHHHHHGGS | ATGCACCACCACCACCACCACGGTGGATCTG |
| [417; | T12M, T14E, | GYMIFVRMLTGK | GCTATATGATTTTCGTACGCATGTTGACTGGA |
| 1067] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLT | GCCTTACTTTTCAAGGGAAGTTGCTGGAGGA |
| | S49L, P38L, | ISPEKMAVLRMQ | CGGTCGCACACTGTCTGACTATAACTTGACTA |
| | R74Q, M1Y, | | TTTCTCCTGAGAAGATGGCCGTACTGCGCATG |
| | V26I, L73M, | | CAA |
| | P69A, L70V, | | |
| | L2M, L62T, | | |
| | D64S, K66E | | |
| CM456 | K6R, T7M, | MHHHHHHGGS | ATGCACCACCACCACCACCACGGTGGATCTG |
| [418; | T12M, T14E, | GYMIFVRMLTGK | GCTATATGATTTTCGTACGCATGTTGACTGGA |
| 1068] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNL | GCCTTACTTTTCAAGGGAAGTTGCTGGAGGA |
| | S49L, P38L, | WISPEKMAVLR | CGGTCGCACACTGTCTGACTATAACTTGTGGA |
| | R74Q, M1Y, | MQ | TTTCTCCTGAGAAGATGGCCGTACTGCGCATG |
| | V26I, L73M, | | CAA |
| | P69A, L70V, | | |
| | L2M, L62W, | | |
| | D64S, K66E | | |
| CM457 | K6R, T7M, | MHHHHHHGGS | ATGCACCACCACCACCACCACGGTGGATCTG |
| [419; | T12M, T14E, | GYMIFVRMLTGK | GCTATATGATTTTCGTACGCATGTTGACTGGA |
| 1069] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLY | GCCTTACTTTTCAAGGGAAGTTGCTGGAGGA |
| | S49L, P38L, | ISPEKMAVLRMQ | CGGTCGCACACTGTCTGACTATAACTTGTATA |
| | R74Q, M1Y, | | TTTCTCCTGAGAAGATGGCCGTACTGCGCATG |
| | V26I, L73M, | | CAA |
| | P69A, L70V, | | |
| | L2M, L62Y, | | |
| | D64S, K66E | | |
| CM458 | K6R, T7M, | MHHHHHHGGS | ATGCACCACCACCACCACCACGGTGGATCTG |
| [420; | T12M, T14E, | GYMIFVRMLTGK | GCTATATGATTTTCGTACGCATGTTGACTGGA |
| 1070] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNL | GCCTTACTTTTCAAGGGAAGTTGCTGGAGGA |
| | S49L, P38L, | VISPEKMAVLRM | CGGTCGCACACTGTCTGACTATAACTTGGTAA |
| | R74Q, M1Y, | Q | TTTCTCCTGAGAAGATGGCCGTACTGCGCATG |
| | V26I, L73M, | | CAA |
| | P69A, L70V, | | |
| | L2M, L62V, | | |
| | D64S, K66E | | |
| CM459 | K6R, T7M, | MHHHHHHGGS | ATGCACCACCACCACCACCACGGTGGATCTG |
| [421; | T12M, T14E, | GYMIFVRMLTGK | GCTATATGATTTTCGTACGCATGTTGACTGGA |
| 1071] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLP | GCCTTACTTTTCAAGGGAAGTTGCTGGAGGA |
| | S49L, P38L, | ISPEKMCFLRMQ | CGGTCGCACACTGTCTGACTATAACTTGCCTA |
| | R74Q, M1Y, | | TTTCTCCTGAGAAGATGTGCTTCCTGCGCATG |
| | V26I, L73M, | | CAA |
| | P69C, L70F, | | |
| | L2M, L62P, | | |
| | D64S, K66E | | |
| CM460 | K6R, T7M, | MHHHHHHGGS | ATGCACCACCACCACCACCACGGTGGATCTG |
| [422; | T12M, T14E, | GYMIFVRMLTGK | GCTATATGATTTTCGTACGCATGTTGACTGGA |
| 1072] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLP | GCCTTACTTTTCAAGGGAAGTTGCTGGAGGA |
| | S49L, P38L, | ISPEKMMFLRM | CGGTCGCACACTGTCTGACTATAACTTGCCTA |
| | R74Q, M1Y, | Q | TTTCTCCTGAGAAGATGATGTTCCTGCGCATG |
| | V26I, L73M, | | CAA |
| | P69M, L70F, | | |
| | L2M, L62P, | | |
| | D64S, K66E | | |
| CM461 | K6R, T7M, | MHHHHHHGGS | ATGCACCACCACCACCACCACGGTGGATCTG |
| [423; | T12M, T14E, | GYMIFVRMLTGK | GCTATATGATTTTCGTACGCATGTTGACTGGA |
| 1073] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLP | GCCTTACTTTTCAAGGGAAGTTGCTGGAGGA |
| | S49L, P38L, | ISPEKMFFLRMQ | CGGTCGCACACTGTCTGACTATAACTTGCCTA |
| | R74Q, M1Y, | | TTTCTCCTGAGAAGATGTTCTTCCTGCGCATG |
| | V26I, L73M, | | CAA |
| | P69F, L70F, | | |
| | L2M, L62P, | | |
| | D64S, K66E | | |
| CM462 | K6R, T7M, | MHHHHHHGGS | ATGCACCACCACCACCACCACGGTGGATCTG |
| [424; | T12M, T14E, | GYMIFVRMLTGK | GCTATATGATTTTCGTACGCATGTTGACTGGA |
| 1074] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLP | GCCTTACTTTTCAAGGGAAGTTGCTGGAGGA |
| | S49L, P38L, | ISPEKMCCLRMQ | CGGTCGCACACTGTCTGACTATAACTTGCCTA |
| | R74Q, M1Y, | | TTTCTCCTGAGAAGATGTGCTGCCTGCGCATG |
| | V26I, L73M, | | CAA |
| | P69C, L70C, | | |
| | L2M, L62P, | | |
| | D64S, K66E | | |
| CM463 | K6R, T7M, | MHHHHHHGGS | ATGCACCACCACCACCACCACGGTGGATCTG |
| [425; | T12M, T14E, | GYMIFVRMLTGK | GCTATATGATTTTCGTACGCATGTTGACTGGA |
| 1075] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLP | GCCTTACTTTTCAAGGGAAGTTGCTGGAGGA |
| | S49L, P38L, | ISPEKMMCLRM | CGGTCGCACACTGTCTGACTATAACTTGCCTA |
| | R74Q, M1Y, | Q | TTTCTCCTGAGAAGATGATGTGCCTGCGCATG |
| | V26I, L73M, | | CAA |
| | P69M, L70C, | | |
| | L2M, L62P, | | |
| | D64S, K66E | | |
| CM464 | K6R, T7M, | MHHHHHHGGS | ATGCACCACCACCACCACCACGGTGGATCTG |
| [426; | T12M, T14E, | GYMIFVRMLTGK | GCTATATGATTTTCGTACGCATGTTGACTGGA |
| 1076] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLP | GCCTTACTTTTCAAGGGAAGTTGCTGGAGGA |
| | S49L, P38L, | ISPEKMFCLRMQ | CGGTCGCACACTGTCTGACTATAACTTGCCTA |
| | R74Q, M1Y, | | TTTCTCCTGAGAAGATGATGTGCCTGCGCATG |
| | V26I, L73M, | | CAA |
| | P69F, L70C, | | |
| | L2M, L62P, | | |
| | D64S, K66E | | |
| CM465 | K6R, T7M, | MHHHHHHGGS | ATGCACCACCACCACCACCACGGTGGATCTG |
| [427; | T12M, T14E, | GYMIFVRMLTGK | GCTATATGATTTTCGTACGCATGTTGACTGGA |
| 1077] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNL | GCCTTACTTTTCAAGGGAAGTTGCTGGAGGA |
| | S49L, P38L, | AISPEKMAMLR | CGGTCGCACACTGTCTGACTATAACTTGGCCA |
| | R74Q, M1Y, | MQ | TTTCTCCTGAGAAGATGATGTGCCTGCGCATG |
| | V26I, L73M, | | CAA |
| | P69A, L70M, | | |
| | L2M, L62A, | | |
| | D64S, K66E | | |
| CM467 | K6R, T7M, | MHHHHHHGGS | ATGCACCACCACCACCACCACGGTGGATCTG |
| [428; | T12M, T14E, | GYMIFVRMLTGK | GCTATATGATTTTCGTACGCATGTTGACTGGA |
| 1078] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLC | GCCTTACTTTTCAAGGGAAGTTGCTGGAGGA |
| | S49L, P38L, | ISPEKMAMLRM | CGGTCGCACACTGTCTGACTATAACTTGTGCA |
| | R74Q, M1Y, | Q | TTTCTCCTGAGAAGATGGCCATGCTGCGCATG |
| | V26I, L73M, | | CAA |
| | P69A, L70M, | | |
| | L2M, L62C, | | |
| | D64S, K66E | | |
| CM468 | K6R, T7M, | MHHHHHHGGS | ATGCACCACCACCACCACCACGGTGGATCTG |
| [429; | T12M, T14E, | GYMIFVRMLTGK | GCTATATGATTTTCGTACGCATGTTGACTGGA |
| 1079] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLT | GCCTTACTTTTCAAGGGAAGTTGCTGGAGGA |
| | S49L, P38L, | ISPEKMAMLRM | CGGTCGCACACTGTCTGACTATAACTTGACTA |
| | R74Q, M1Y, | Q | TTTCTCCTGAGAAGATGGCCATGCTTGCGCATG |
| | V26I, L73M, | | CAA |
| | P69A, L70M, | | |
| | L2M, L62T, | | |
| | D64S, K66E | | |
| CM469 | K6R, T7M, | MHHHHHHGGS | ATGCACCACCACCACCACCACGGTGGATCTG |
| [430; | T12M, T14E, | GYMIFVRMLTGK | GCTATATGATTTTCGTACGCATGTTGACTGGA |
| 1080] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNL | GCCTTACTTTTCAAGGGAAGTTGCTGGAGGA |
| | S49L, P38L, | VISPEKMAMLR | CGGTCGCACACTGTCTGACTATAACTTGGTAA |
| | R74Q, M1Y, | MQ | TTTCTCCTGAGAAGATGGCCATGCTGCGCATG |
| | V26I, L73M, | | CAA |
| | P69A, L70M, | | |
| | L2M, L62V, | | |
| | D64S, K66E | | |
| CM478 | K6R, T7M, | MHHHHHHGGS | ATGCACCATCACCACCACCACGGTGGATCTG |
| [431; | T12M, T14E, | GYAIFVRMLTGK | GCTATGCCATTTTCGTACGCATGTTGACTGGA |
| 1081] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLT | GCCTTACTTTTCAAGGGAAGTTGCTGGAGGA |
| | S49L, P38L, | ISPEKMAVLRMQ | CGGTCGCACACTGTCTGACTATAACTTGACTA |
| | R74Q, M1Y, | | TTTCTCCTGAGAAGATGGCCGTACTGCGCATG |
| | V26I, L73M, | | CAA |
| | P69A, L70V, | | |
| | L2A, L62T, | | |
| | D64S, K66E | | |
| CM479 | K6R, T7M, | MHHHHHHGGS | ATGCACCATCACCACCACCACGGTGGATCTG |
| [432; | T12M, T14E, | GYRIFVRMLTGK | GCTATCGCATTTTCGTACGCATGTTGACTGGA |
| 1082] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLT | GCCTTACTTTTCAAGGGAAGTTGCTGGAGGA |
| | S49L, P38L, | ISPEKMAVLRMQ | CGGTCGCACACTGTCTGACTATAACTTGACTA |
| | R74Q, M1Y, | | TTTCTCCTGAGAAGATGGCCGTACTGCGCATG |
| | V26I, L73M, | | CAA |
| | P69A, L70V, | | |
| | L2R, L62T, | | |
| | D64S, K66E | | |
| CM480 | K6R, T7M, | MHHHHHHGGS | ATGCACCATCACCACCACCACGGTGGATCTG |
| [433; | T12M, T14E, | GYNIFVRMLTGK | GCTATAATATTTTCGTACGCATGTTGACTGGA |
| 1083] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLT | GCCTTACTTTTCAAGGGAAGTTGCTGGAGGA |
| | S49L, P38L, | ISPEKMAVLRMQ | CGGTCGCACACTGTCTGACTATAACTTGACTA |
| | R74Q, M1Y, | | TTTCTCCTGAGAAGATGGCCGTACTGCGCATG |
| | V26I, L73M, | | CAA |
| | P69A, L70V, | | |
| | L2N, L62T, | | |
| | D64S, K66E | | |
| CM481 | K6R, T7M, | MHHHHHHGGS | ATGCACCATCACCACCACCACGGTGGATCTG |
| [434; | T12M, T14E, | GYDIFVRMLTGK | GCTATGAIATTTTCGTACGCATGTTGACTGGA |
| 1084] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLT | GCCTTACTTTTCAAGGGAAGTTGCTGGAGGA |
| | S49L, P38L, | ISPEKMAVLRMQ | CGGTCGCACACTGTCTGACTATAACTTGACTA |
| | R74Q, M1Y, | | TTTCTCCTGAGAAGATGGCCGTACTGCGCATG |
| | V26I, L73M, | | CAA |
| | P69A, L70V, | | |
| | L2D, L62T, | | |
| | D64S, K66E | | |
| CM482 | K6R, T7M, | MHHHHHHGGS | ATGCACCATCACCACCACCACGGTGGATCTG |
| [435; | T12M, T14E, | GYCTFVRMLTGK | GCTATTGCATTTTCGTACGCATGTTGACTGGA |
| 1085] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLT | GCCTTACTTTTCAAGGGAAGTTGCTGGAGGA |
| | S49L, P38L, | ISPEKMAVLRMQ | CGGTCGCACACTGTCTGACTATAACTTGACTA |
| | R74Q, M1Y, | | TTTCTCCTGAGAAGATGGCCGTACTGCGCATG |
| | V26I, L73M, | | CAA |
| | P69A, L70V, | | |
| | L2C, L62T, | | |
| | D64S, K66E | | |
| CM483 | K6R, T7M, | MHHHHHHGGS | ATGCACCATCACCACCACCACGGTGGATCTG |
| [436; | T12M, T14E, | GYEIFVRMLTGK | GCTATGAGATTTTCGTACGCATGTTGACTGGA |
| 1086] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLT | GCCTTACTTTTCAAGGGAAGTTGCTGGAGGA |
| | S49L, P38L, | ISPEKMAVLRMQ | CGGTCGCACACTGTCTGACTATAACTTGACTA |
| | R74Q, M1Y, | | TTTCTCCTGAGAAGATGGCCGTACTGCGCATG |
| | V26I, L73M, | | CAA |
| | P69A, L70V, | | |
| | L2E, L62T, | | |
| | D64S, K66E | | |
| CM484 | K6R, T7M, | MHHHHHHGGS | ATGCACCATCACCACCACCACGGTGGATCTG |
| [437; | T12M, T14E, | GYQIFVRMLTGK | GCTATCAAATTTTCGTACGCATGTTGACTGGA |
| 1087] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLT | GCCTTACTTTTCAAGGGAAGTTGCTGGAGGA |
| | S49L, P38L, | ISPEKMAVLRMQ | CGGTCGCACACTGTCTGACTATAACTTGACTA |
| | R74Q, M1Y, | | TTTCTCCTGAGAAGATGGCCGTACTGCGCATG |
| | V26I, L73M, | | CAA |
| | P69A, L70V, | | |
| | L20, L62T, | | |
| | D64S, K66E | | |
| CM485 | K6R, T7M, | MHHHHHHGGS | ATGCACCATCACCACCACCACGGTGGATCTG |
| [438; | T12M, T14E, | GYGIFVRMLTGK | GCTATGGGATTTTCGTACGCATGTTGACTGGA |
| 1088] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLT | GCCTTACTTTTCAAGGGAAGTTGCTGGAGGA |
| | S49L, P38L, | ISPEKMAVLRMQ | CGGTCGCACACTGTCTGACTATAACTTGACTA |
| | R74Q, M1Y, | | TTTCTCCTGAGAAGATGGCCGTACTGCGCATG |
| | V26I, L73M, | | CAA |
| | P69A, L70V, | | |
| | L2G, L62T, | | |
| | D64S, K66E | | |
| CM486 | K6R, T7M, | MHHHHHHGGS | ATGCACCATCACCACCACCACGGTGGATCTG |
| [439; | T12M, T14E, | GYHIFVRMLTGK | GCTATCATATTTTCGTACGCATGTTGACTGGA |
| 1089] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K631, A44T, | LEDGRTLSDYNLT | GCCTTACTTTTCAAGGGAAGTTGCTGGAGGA |
| | S49L, P38L, | ISPEKMAVLRMQ | CGGTCGCACACTGTCTGACTATAACTTGACTA |
| | R74Q, M1Y, | | TTTCTCCTGAGAAGATGGCCGTACTGCGCATG |
| | V26I, L73M, | | CAA |
| | P69A, L70V, | | |
| | L2H, L62T, | | |
| | D64S, K66E | | |
| CM487 | K6R, T7M, | MHHHHHHGGS | ATGCACCATCACCACCACCACGGTGGATCTG |
| [440; | T12M, T14E, | GYIIFVRMLTGK | GCTATATTATTTTCGTACGCATGTTGACTGGA |
| 1090] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLT | GCCTTACTTTTCAAGGGAAGTTGCTGGAGGA |
| | S49L, P38L, | ISPEKMAVLRMQ | CGGTCGCACACTGTCTGACTATAACTTGACTA |
| | R74Q, M1Y, | | TTTCTCCTGAGAAGATGGCCGTACTGCGCATG |
| | V26I, L73M, | | CAA |
| | P69A, L70V, | | |
| | L2I, L62T, | | |
| | D64S, K66E | | |
| CM488 | K6R, T7M, | MHHHHHHGGS | ATGCACCATCACCACCACCACGGTGGATCTG |
| [441; | T12M, T14E, | GYKIFVRMLTGK | GCTATAAGATTTTCGTACGCATGTTGACTGGA |
| 1091] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLT | GCCTTACTTTTCAAGGGAAGTTGCTGGAGGA |
| | S49L, P38L, | ISPEKMAVLRMQ | CGGTCGCACACTGTCTGACTATAACTTGACTA |
| | R74Q, M1Y, | | TTTCTCCTGAGAAGATGGCCGTACTGCGCATG |
| | V26I, L73M, | | CAA |
| | P69A, L70V, | | |
| | L2K, L62T, | | |
| | D64S, K66E | | |
| CM489 | K6R, T7M, | MHHHHHHGGS | ATGCACCATCACCACCACCACGGTGGATCTG |
| [442; | T12M, T14E, | GYLIFVRMLTGK | GCTATTTGATTTTCGTACGCATGTTGACTGGA |
| 1092] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLT | GCCTTACTTTTCAAGGGAAGTTGCTGGAGGA |
| | S49L, P38L, | ISPEKMAVLRMQ | CGGTCGCACACTGTCTGACTATAACTTGACTA |
| | R74Q, M1Y, | | TTTCTCCTGAGAAGATGGCCGTACTGCGCATG |
| | V26I, L73M, | | CAA |
| | P69A, L70V, | | |
| | L62T, D64S, | | |
| | K66E | | |
| CM490 | K6R, T7M, | MHHHHHHGGS | ATGCACCATCACCACCACCACGGTGGATCTG |
| [443; | T12M, T14E, | GYFIFVRMLTGK | GCTATTTCATTTTCGTACGCATGTTGACTGGA |
| 1093] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLT | GCCTTACTTTTCAAGGGAAGTTGCTGGAGGA |
| | S49L, P38L, | ISPEKMAVLRMQ | CGGTCGCACACTGTCTGACTATAACTTGACTA |
| | R74Q, M1Y, | | TTTCTCCTGAGAAGATGGCCGTACTGCGCATG |
| | V26I, L73M, | | CAA |
| | P69A, L70V, | | |
| | L2F, L62T, | | |
| | D64S, K66E | | |
| CM491 | K6R, T7M, | MHHHHHHGGS | ATGCACCATCACCACCACCACGGTGGATCTG |
| [444; | T12M, T14E, | GYSIFVRMLTGK | GCTATTCTATTTTCGTACGCATGTTGACTGGA |
| 1094] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLT | GCCTTACTTTTCAAGGGAAGTTGCTGGAGGA |
| | S49L, P38L, | ISPEKMAVLRMQ | CGGTCGCACACTGTCTGACTATAACTTGACTA |
| | R74Q, M1Y, | | TTTCTCCTGAGAAGATGGCCGTACTGCGCATG |
| | V26I, L73M, | | CAA |
| | P69A, L70V, | | |
| | L2S, L62T, | | |
| | D64S, K66E | | |
| CM492 | K6R, T7M, | MHHHHHHGGS | ATGCACCATCACCACCACCACGGTGGATCTG |
| [445; | T12M, T14E, | GYTIFVRMLTGK | GCTATACTATTTTCGTACGCATGTTGACTGGA |
| 1095] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLT | GCCTTACTTTTCAAGGGAAGTTGCTGGAGGA |
| | S49L, P38L, | ISPEKMAVLRMQ | CGGTCGCACACTGTCTGACTATAACTTGACTA |
| | R74Q, M1Y, | | TTTCTCCTGAGAAGATGGCCGTACTGCGCATG |
| | V26I, L73M, | | CAA |
| | P69A, L70V, | | |
| | L2T, L62T, | | |
| | D64S, K66E | | |
| CM493 | K6R, T7M, | MHHHHHHGGS | ATGCACCATCACCACCACCACGGTGGATCTG |
| [446; | T12M, T14E, | GYWIFVRMLTGK | GCTATTGGATTTTCGTACGCATGTTGACTGGA |
| 1096] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLT | GCCTTACTTTTCAAGGGAAGTTGCTGGAGGA |
| | S49L, P38L, | ISPEKMAVLRMQ | CGGTCGCACACTGTCTGACTATAACTTGACTA |
| | R74Q, M1Y, | | TTTCTCCTGAGAAGATGGCCGTACTGCGCATG |
| | V26I, L73M, | | CAA |
| | P69A, L70V, | | |
| | L2W, L62T, | | |
| | D64S, K66E | | |
| CM494 | K6R, T7M, | MHHHHHHGGS | ATGCACCATCACCACCACCACGGTGGATCTG |
| [447; | T12M, T14E, | GYYIFVRMLTGK | GCTATTATATTTTCGTACGCATGTTGACTGGA |
| 1097] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLT | GCCTTACTTTTCAAGGGAAGTTGCTGGAGGA |
| | S49L, P38L, | ISPEKMAVLRMQ | CGGTCGCACACTGTCTGACTATAACTTGACTA |
| | R74Q, M1Y, | | TTTCTCCTGAGAAGATGGCCGTACTGCGCATG |
| | V26I, L73M, | | CAA |
| | P69A, L70V, | | |
| | L2Y, L62T, | | |
| | D64S, K66E | | |
| CM495 | K6R, T7M, | MHHHHHHGGS | ATGCACCATCACCACCACCACGGTGGATCTG |
| [448; | T12M, T14E, | GYVIFVRMLTGK | GCTATGTAATTTTCGTACGCATGTTGACTGGA |
| 1098] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLT | GCCTTACTTTTCAAGGGAAGTTGCTGGAGGA |
| | S49L, P38L, | ISPEKMAVLRMQ | CGGTCGCACACTGTCTGACTATAACTTGACTA |
| | R74Q, M1Y, | | TTTCTCCTGAGAAGATGGCCGTACTGCGCATG |
| | V26I, L73M, | | CAA |
| | P69A, L70V, | | |
| | L2V, L62T, | | |
| | D64S, K66E | | |
| CM496 | K6R, T7M, | MHHHHHHGGS | ATGCACCATCACCACCACCACGGTGGATCTG |
| [449; | T12M, T14E, | GYPIFVRMLTGK | GCTATCCTATTTTCGTACGCATGTTGACTGGA |
| 1099] | K33H, A46Q, | MIELEVEPSDTIE | AAGATGATCGAGTTGGAAGTGGAGCCTTCCG |
| | S65P, L67K, | NIKAKIQDHEGIP | ATACTATCGAGAATATTAAGGCCAAAATCCAA |
| | H68M, I61L, | LDQQRLTFQGKL | GATCATGAAGGGATTCCTTTGGATCAACAAC |
| | K63I, A44T, | LEDGRTLSDYNLT | GCCTTACTTTTCAAGGGAAGTTGCTGGAGGA |
| | S49L, P38L, | ISPEKMAVLRMQ | CGGTCGCACACTGTCTGACTATAACTTGACTA |
| | R74Q, M1Y, | | TTTCTCCTGAGAAGATGGCCGTACTGCGCATG |
| | V26I, L73M, | | CAA |
| | P69A, L70V, | | |
| | L2P, L62T, | | |
| | D64S, K66E | | |

^{a}The SEQ ID NOS shown in brackets correspond to the protein amino acid SEQ ID NO, followed by the DNA nucleic acid SEQ ID No.

### Example 5. Ubiquitin Variants Targeting 53BP1 Provide an Additional Benefit to HDR when Used in Conjunction with NHEJ Inhibitors

To test if ubiquitin variants targeting 53BP1 provide a benefit when used in conjunction with small molecule inhibitors reported to boost HDR we tested if the rate of HDR using a DNA-dependent protein kinase (DNA-PK) inhibitor, IDT Enhancer (IDT-E or Alt-R HDR Enhancer), was further increased by using it in combination with CM1. DNA-PK is a critical protein complex in the NHEJ pathway, by inhibiting DNA-PK these small molecules bias the cell towards use of homologous recombination instead of NHEJ to repair double strand breaks induced by CRISPR/Cas9 and other nucleases thereby facilitating gene editing. Notably, 53BP1 recruitment is not dependent on the kinase activity of DNA-PK and is instead recruited through an ATM dependent pathway [29, 30]. Further, 53BP1 recruitment and formation of 53BP1 foci is often used to visualize the presence of double strand breaks, including in the presence of DNA-PK inhibitors which can cause 53BP1 foci to persist for a greater period due to inhibition of the normally rapid repair through the NHEJ pathway [27, 31]. We hypothesized that inhibition of 53BP1 may provide an additional benefit when used in conjunction with inhibitors of common NHEJ pathway targets such as DNA-PK and DNA-ligase IV due to the ability of inhibitors of 53BP1 to enhance HDR not just through a negative effect on NHEJ but also promoting HDR by facilitating end resection.

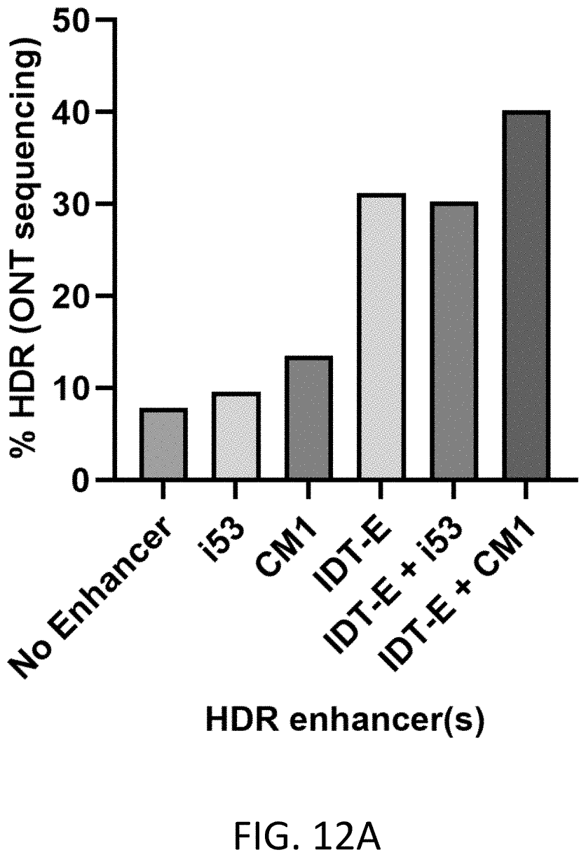

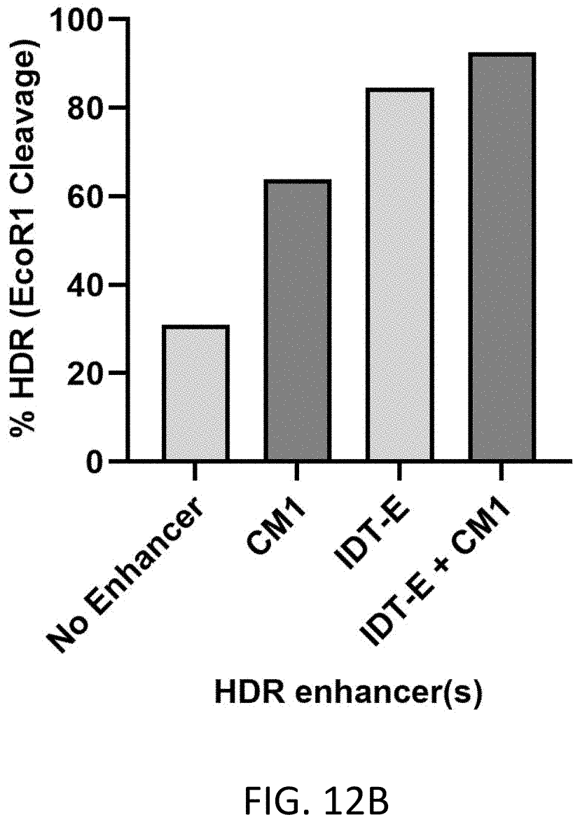

We tested if our ubiquitin variants provided a further benefit over inhibition of common NHEJ pathway targets alone by using the DNA-PK inhibitor IDT enhancer (IDT-E) in combination with CM1 in the context of both large and small inserts (Table 7). The results are shown in FIG. 12. Both IDT-E and CM1 were able to individual increase rates of HDR using both donors types, however higher HDR rates were achieved when both were used together than either inhibitor alone. Without limiting the claimed subject matter to a particular mode or mechanism of action, we hypothesize that our ubiquitin variants targeting 53BP1 will be a useful in facilitating increased HIDR when used in combination with other inhibitors of NHEJ pathway components.

TABLE 7
Gene, protospacer, targets, and donor sequences.

| Gene | | Coor- | |
|---|---|---|---|
| [SEQ ID | | dinates | |
| NO:]^{a} | protospacer | (hg38) | Donor Sequence |
| SERPINC1 | ACCTCTG | chr1: | /Alt-R-HDR1/A*T*TCCAATGTGATAGGAACTGTAACCTCTGGA |
| [1101; | GAAAAAG | 173,917,213- | AAAAGGTAGAATTCAGAGGGGTGAGCTTTCCCCTTGCCTGC |
| 1103] | GTAAGA | 173, 917,232 | CCCTACTGGGT*T*T/Alt-R-HDR2/ |
| MET | CAAAGTCC | chr7: | /Alt-R-HDR1/T*G*TGTGGTGAGCGCCCTGGGAGCCAAAGTCC |
| [1104; | TTTCATCTG | 116,699,630- | TTTCATCTGGAATTCTAAAGGACCGGTTCATCAACTTCTTTG |
| 1105] | TAA | 116,699,649 | TAGGCAATACC*A*T/Alt-R-HDR2/ |
| HPRT1 | AATTATGG | chrX: | /Alt-R-HDR1/A*A*AGACTATGAAATGGAGAGCTAAATTATGGGGA |
| [1106; | GGATTACT | 134,498,212- | TTACTAGAATTCGGAAGGGGCAGCAATGAGTTGACACTACAGACA |
| 1107] | AGGA | 134,498,231 | AGGCA*C*T/Alt-R-HDR2/ |
| CLTA | GAACGGA | chr9: | GTCGTACCGACTGGTAGATGACAGCAAACCTGTTCCCTTTTCGGCTC |
| [1108; | TCCAGCT | 36,191,058- | TGCAACACCGCCTAGACCGACCGGATACACGGGTAGGGCTTCCGCT |
| 1109] | CAGCCA | 36,191,077 | TTACCCGTCTCCCTCCTGGCGCTTGTCCTCCTCTCCCAGTCGGCACCA |
| | | | CAGCGGTGGCTGCCGGGCGTGGTGTCGGTGGGTCGGTTGGTTTTT |
| | | | GTCTCACCGTTGGTGTCCGTGCCGTTCAGTTGCCCGCCATGGCTGG |
| | | | ATCTGGTGGTACTAGTGGAAGCAAGGGTGAGGAGCTGTTCACCGG |
| | | | AGTGGTGCCTATCCTGGTCGAGCTGGACGGCGACGTAAACGGTCA |
| | | | CAAGTTCAGCGTGCGTGGTGAGGGCGAGGGCGATGCCACCAACGG |
| | | | CAAGCTGACCCTGAAGTTCATCTGCACCACTGGCAAGCTGCCTGTTC |
| | | | CATGGCCAACCCTCGTGACTACACTGACCTACGGCGTTCAGTGCTTC |
| | | | AGCCGTTACCCTGACCATATGAAGCGTCACGACTTCTTCAAGTCTGC |
| | | | CATGCCTGAAGGCTACGTCCAGGAGCGTACCATCAGCTTCAAGGAC |
| | | | GATGGCACCTACAAGACTCGTGCCGAGGTGAAGTTCGAGGGTGAC |
| | | | ACCCTGGTGAACCGCATCGAGCTGAAGGGTATCGACTTCAAGGAG |
| | | | GACGGCAACATCCTGGGTCACAAGCTGGAGTACAACTTCAACAGCC |
| | | | ACAACGTCTATATCACCGCCGACAAGCAGAAGAACGGCATCAAGG |
| | | | CCAACTTCAAGATTCGTCACAACGTGGAGGACGGTAGCGTGCAGCT |
| | | | CGCAGACCACTACCAGCAGAACACGCCTATCGGCGACGGTCCAGTG |
| | | | TTGCTGCCAGACAACCACTACCTGAGCACCCAGTCCGTGCTGAGCA |
| | | | AAGACCCGAACGAGAAGCGCGATCACATGGTCCTGCTGGAGTTCG |
| | | | TGACCGCAGCCGGTATCACTGGAACCGGTGCTGGAAGTGGTGAGC |
| | | | TGGATCCGTTCGGCGCCCCTGCCGGCGCCCCTGGCGGTCCCGCGCT |
| | | | GGGGAACGGAGTGGCCGGCGCCGGCGAAGAAGACCCGGCTGCGG |
| | | | CCTTCTTGGCGCAGCAAGAGAGCGAGATTGCGGGCATCGAGAACG |
| | | | ACGAGGCCTTCGCCATCCTGGACGGCGGCGCCCCCGGGCCCCAGC |
| | | | CGCACGGCGAGCCGCCGATCCGAAAACGGGCGTATAGTCGAGACC |

^{a}The SEQ D NOS shown in brackets correspond to the protospacer SEQ ID NO, followed by the Donor Seq uence SEQ ID NO.

### Example 6. Screening of Amino Acid Substitutions at Position 2 Reveals an Additional Beneficial Mutation at Position 2

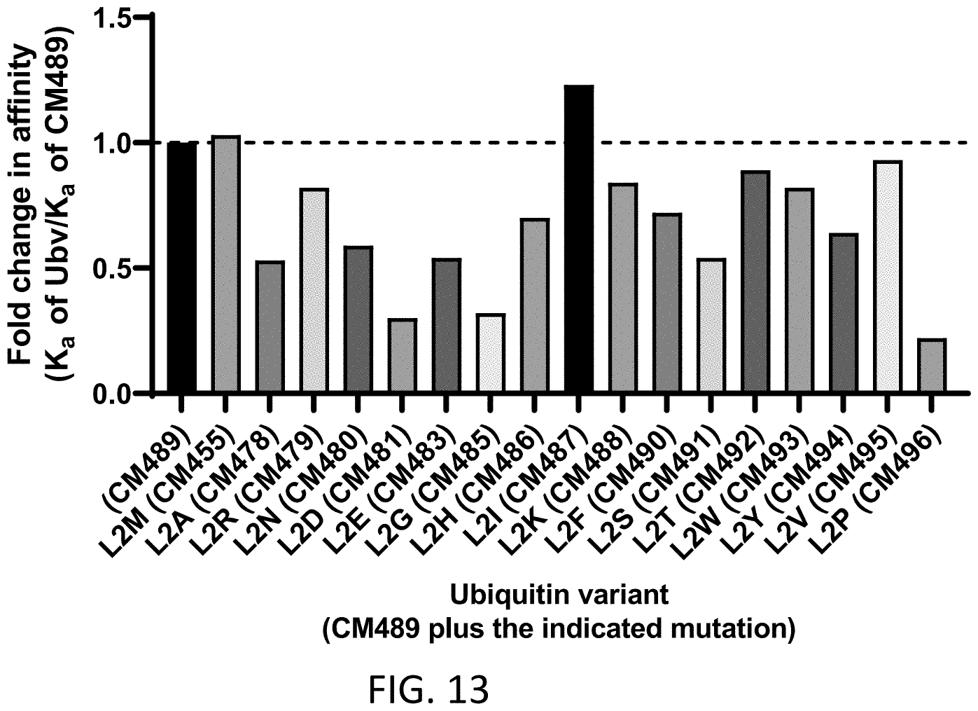

Testing of additional mutations identified a variant with improved affinity over that of the previously described CM455. In order to determine if the amino acid change made at position 2 (L2M) in CM455 relative to i53 was the optimal amino acid change at that position, we screened additional amino acid changes for their effect on the affinity for binding 53BP1. The results are shown in FIG. 13. The fold change in affinity is measured as the association constant (KA) of the ubiquitin variant being tested, divided by the KA of the reference ubiquitin variant (CM489), as determined by calculating each affinity for binding a fragment of 53BP1 using biolayer interferometry (BLI). The BLI steady-state response versus 53BP1 fragment concentration was plotted in prism to calculate the Kd using a one site-specific binding nonlinear fit model. If the affinity of a ubiquitin variant being tested is higher (binding is tighter) than for the reference ubiquitin variant, then the fold change in affinity will be >1. Of the mutations tested, the majority were shown to be detrimental, resulting in worse affinity for 53BP1 than CM455. Compared to CM489 which has the original Q2L mutation at position 2 (relative to WT ubiquitin), the L2M mutation (Q2M relative to wild-type ubiquitin) identified from our previously described screen as the least detrimental mutation at position 2 provides a similar level of affinity as the Q2L mutation, however our L2I mutation (Q2I relative to WT ubiquitin) results in higher affinity than the L2M of CM455. Therefore, switching from L2M to L2I in CM455 may result in a ubiquitin variant (CM487) with improved ability to enhance rates of HDR.

### Example 7. Tag-Free CM1 (CM1tf) Boosts HDR to the Same Degree as 6×His-Tagged CM1

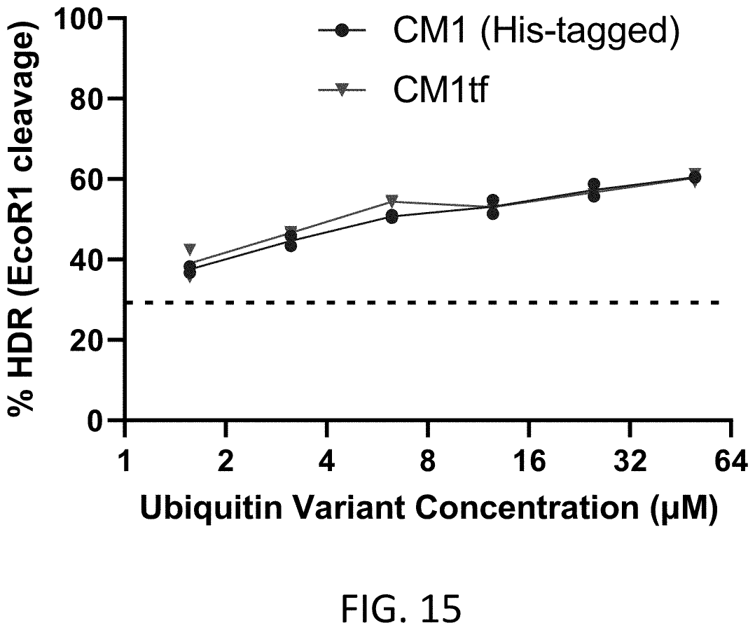

A tag-free version of CM1 (CM1tf, SEQ ID NO:482) was compared with the His6-tagged version of CM1 (SEQ ID NO:241) for their ability to enhance HDR in HEK293 cells as has been described in previous examples. Briefly, 2 uM Cas9 RNP targeting a site in HPRT1 and 2 uM ssDNA donor containing 40 bp homology arms flanking a 6 bp EcoR1 cut site insert sequence were delivered into HEK293 cells with varying amounts of CM1tf (CM1tf, SEQ ID NO:482) or His-tagged CM1 (CM1; SEQ ID NO:241) using Lonza nucleofection. Genomic DNA was isolated after 48 hours, and editing was measured using an EcoR1 cleavage assay. The results are shown in FIG. 15. We found that the ability of the CM1 variant lacking a His-tag (CM1tf, SEQ ID NO:482) to enhance HDR is equivalent to that of His-tagged CM1 (CM1; SEQ ID NO:241).

### Example 8. Mode of Delivery of an Ubv Via mRNA or Vector-Mediated Expression is Effective at Enhancing HDR Rates

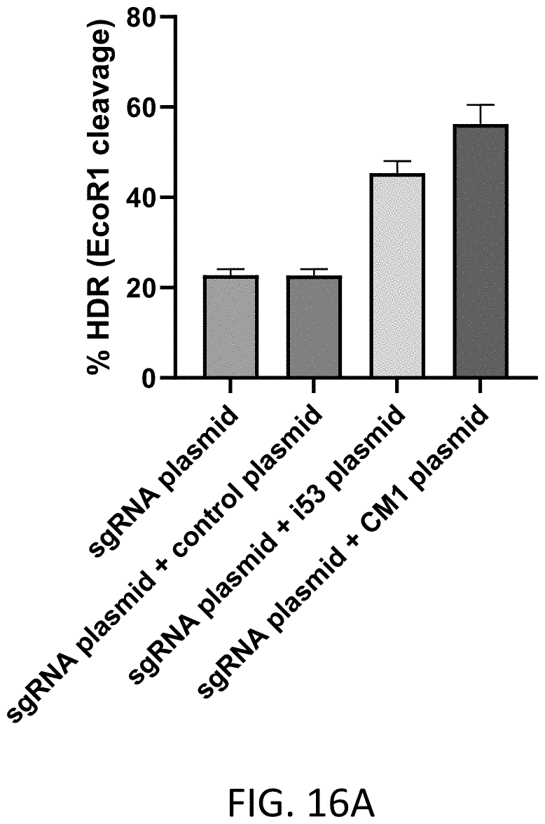

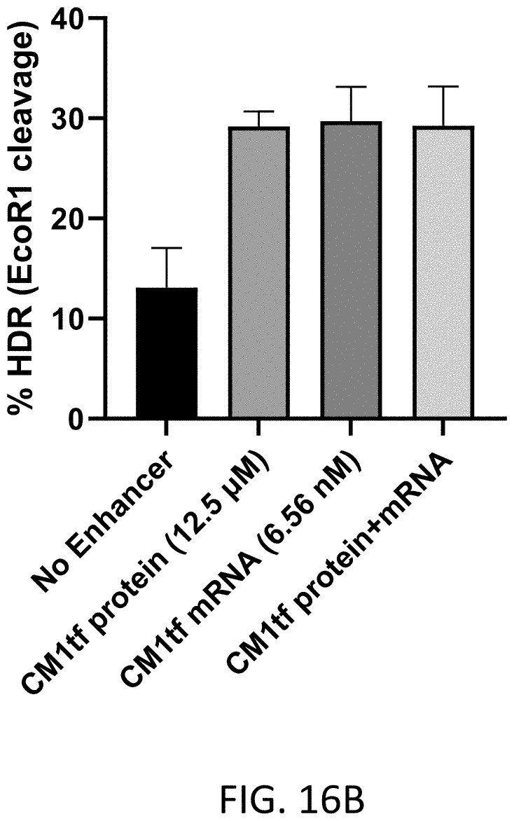

In order to test if CM1 is effective at increasing HDR rates when delivered in other forms, plasmid or mRNA encoding CM1 was introduced into cells and the effects on HDR rates were analyzed. To test the effectiveness of CM1 delivered as plasmid, 154 ng of plasmid encoding His-tagged i53, His-tagged CM1, or a crRNA for LbCas12a was co-delivered with 154 ng of plasmid encoding sgRNA targeting HPRT1 into Jurkat cells by Lonza nucleofection using SF buffer and program DS-150. After 72 hours, genomic DNA was extracted using QuickExtract (Lucigen) and editing was analyzed by PCR amplification of the HPRT1 target site followed by EcoR1 restriction enzyme digestion. Digested product was run on a Fragment Analyzer (AATI). The results are shown in FIG. 16A.

Use of plasmid encoding i53 or CM1 resulted in an increase in HDR rates, with CM1 causing a larger increase in HDR rate. In order to test if CM1 is effective when delivered as mRNA, mRNA encoding CM1tf or CM1tf protein (12.5 μM) was delivered with 2 μM Cas9 RNP targeting HPRT1 and 2 μM HPRT1 EcoR1 cut site ssDNA donor by Lonza nucleofection (SE solution, pulse code CL-120). The indicated mRNA concentration (6.56 nM) was calculated using the commonly used 40 ug/ml for an OD260 of 1 absorbance estimate for ssRNA. Using a sequence specific extinction coefficient, the concentration was calculated as 4.61 nM. After 48 hours genomic DNA was extracted and the rate of HDR was analyzed as described previously. The results are shown in FIG. 16B.

Introduction of CM1tf as either protein or mRNA provided a similar level of boost in HDR rates over the no enhancer control. No additional benefit was observed when CM1tf mRNA and protein were added together, however there may be some benefit to adding them in combination in other cell types or with other types of donor DNA. The CM1tf mRNA was generated from PCR product from a human codon optimized CM1tf expression vector (made by IDT) using the HiScribe T7 ARCA kit (NEB) and Monarch RNA cleanup columns (NEB). The poly-A tail was encoded in the PCR product by addition of a poly-T sequence to the reverse primer (Table 8).

TABLE 8
Sequences associated with CMltf mRNA production:

| | |
|---|---|
| reverse primer to | TTTTTTTTTTTTTTTTTTTTTTTTTTTTTTTTTTTTTTTTTTTTT |
| generate DNA | TTTTTTTTTTTTTTTTTTTTTTTTTTTTTTTTTTTTTTTTTTTTT |
| template for mRNA | TTTTTTTTTTTTTTTTTTTTTTTTTTTTTTTAGAAGGCACAGT |
| production | CGAGGCT |
| [SEQ ID NO: 1110] | |
| Forward primer to | CCACTGCTTACTGGCTTATCGAAAT |
| generate DNA | |
| template for mRNA | |
| production | |
| [SEQ ID NO: 1111] | |
| PCR amplified | CCACTGCTTACTGGCTTATCGAAATTAATACGACTCACTATAGGGAGAC |
| sequence | CCAAGCTGGCTAGCGTTTAAACGGGCCCTCTAGACTCGAGCGGCCGCC |
| (double underline | ACCATGCTGATCTTCGTGAGAATGCTGACCGGCAAGATGATCGAACTG |
| indicates transcription | GAAGTGGAACCCAGCGACACCATCGAGAACGTGAAGGCCAAAATCCAG |
| start site) | GACCACGAGGGCATCCCTCCTGACCAGCAGAGACTGGCCTTTCAGGGA |
| (underlined region is | AAGTCCCTGGAAGATGGAAGAACCCTGAGCGACTACAACATCCTGAAG |
| the open reading | GACCCTAAGAAGATGCCACTGCTGAGACTGAGATGATCAGCCTCGACT |
| frame for CM1tf) | GTGCCTTCTAAAAAAAAAAAAAAAAAAAAAAAAAAAAAAAAAAAAAAA |
| [SEQ ID NO: 1112] | aaaaaaaaaaaaaaaaaaaaaaaaaaaaaaaaaaaaaaaaaaaaaaa |
| | aaaaaaaaaaaaaaaaaaaaaaaaaaaaaaaaaaa |

### Example 9. Sequences

A summary of amino acid and DNA sequences is presented in Table 9.

TABLE 9
Summary of Ubiquitin, i53 and Tag-free versions of Ubvs Sequences

| Name | Amino acid | | |
|---|---|---|---|
| [SEQ ID | changes | Protein | |
| NOS]^{a} | relative to i53 | Sequence | DNA sequence |
| Ubiquitin | C-terminal GG | MQIFVKTLTG | ATGCAGATTTTCGTGAAAACCCTTACGGGGA |
| [1; 666] | Q2L, I44A, | KTITLEVEPS | AGACCATCACCCTCGAGGTTGAACCCTCGGA |
| | Q49S, Q62L, | DTIENVKAKI | TACGATAGAAAATGTAAAGGCCAAGATCCAG |
| | E64D, T66K, | QDKEGIPPDQ | GATAAGGAAGGAATTCCTCCTGATCAGCAGA |
| | L69P, and V70L | QRLIFAGKQL | GACTGATCTTTGCTGGCAAGCAGCTGGAAGA |
| | | EDGRTLSDYN | TGGACGTACTTTGTCTGACTACAATATTCAAA |
| | | IQKESTLHLV | AGGAGTCTACTCTTCATCTTGTGTTGAGACTT |
| | | LRLRGG | CGTGGTGGT |
| i53 | None | MLIFVKTLTGKTI | ATGTTGATTTTCGTAAAGACGTTGACTGGAAA |
| [2; 667] | | TLEVEPSDTIENV | GACTATCACTTTGGAAGTGGAGCCTTCCGATA |
| | | KAKIQDKEGIPPD | CTATCGAGAATGTTAAGGCCAAAATCCAAGA |
| | | QQRLAFAGKSLE | TAAGGAAGGGATTCCTCCAGATCAACAACGC |
| | | DGRTLSDYNILKD | CTTGCTTTTGCCGGGAAGAGCCTGGAGGACG |
| | | SKLHPLLRLR | GTCGCACACTGTCTGACTATAACATTCTTAAA |
| | | | GATTCTAAATTGCATCCACTGCTGCGCTTGCG |
| | | | T |
| i53 DM | P69L, L70V | MLIFVKTLTGKTI | ATGTTGATTTTCGTAAAGACGTTGACTGGAAA |
| [451; 668] | | TLEVEPSDTIENV | GACTATCACTTTGGAAGTGGAGCCTTCCGATA |
| | | KAKIQDKEGIPPD | CTATCGAGAATGTTAAGGCCAAAATCCAAGA |
| | | QQRLAFAGKSLE | TAAGGAAGGGATTCCTCCAGATCAACAACGC |
| | | DGRTLSDYNILKD | CTTGCTTTTGCCGGGAAGAGCCTGGAGGACG |
| | | SKLHLVLRLR | GTCGCACACTGTCTGACTATAACATTCTTAAA |
| | | | GATTCTAAATTGCATCTGGTTCTGCGCTTGCG |
| | | | T |
| i53 K6R | K6R | MLIFVRTLTGKTI | ATGTTGATTTTCGTACGCACGTTGACTGGAAA |
| [452; 669] | | TLEVEPSDTIENV | GACTATCACTTTGGAAGTGGAGCCTTCCGATA |
| | | KAKIQDKEGIPPD | CTATCGAGAATGTTAAGGCCAAAATCCAAGA |
| | | QQRLAFAGKSLE | TAAGGAAGGGATTCCTCCAGATCAACAACGC |
| | | DGRTLSDYNILKD | CTTGCTTTTGCCGGGAAGAGCCTGGAGGACG |
| | | SKLHPLLRLR | GTCGCACACTGTCTGACTATAACATTCTTAAA |
| | | | GATTCTAAATTGCATCCACTGCTGCGCTTGCG |
| | | | T |
| i53 T14E | T14E | MLIFVKTLTGKTI | ATGTTGATTTTCGTAAAGACGTTGACTGGAAA |
| [453; 670] | | ELEVEPSDTIENV | GACTATCGAGTTGGAAGTGGAGCCTTCCGAT |
| | | KAKIQDKEGIPPD | ACTATCGAGAATGTTAAGGCCAAAATCCAAG |
| | | QQRLAFAGKSLE | ATAAGGAAGGGATTCCTCCAGATCAACAACG |
| | | DGRTLSDYNILKD | CCTTGCTTTTGCCGGGAAGAGCCTGGAGGAC |
| | | SKLHPLLRLR | GGTCGCACACTGTCTGACTATAACATTCTTAA |
| | | | AGATTCTAAATTGCATCCACTGCTGCGCTTGC |
| | | | GT |
| i53 K33A | K33A | MLIFVKTLTGKTI | ATGTTGATTTTCGTAAAGACGTTGACTGGAAA |
| [454; 671] | | TLEVEPSDTIENV | GACTATCACTTTGGAAGTGGAGCCTTCCGATA |
| | | KAKIQDAEGIPPD | CTATCGAGAATGTTAAGGCCAAAATCCAAGA |
| | | QQRLAFAGKSLE | TGCCGAAGGGATTCCTCCAGATCAACAACGC |
| | | DGRTLSDYNILKD | CTTGCTTTTGCCGGGAAGAGCCTGGAGGACG |
| | | SKLHPLLRLR | GTCGCACACTGTCTGACTATAACATTCTTAAA |
| | | | GATTCTAAATTGCATCCACTGCTGCGCTTGCG |
| | | | T |
| i53 A46Q | A46Q | MLIFVKTLTGKTI | ATGTTGATTTTCGTAAAGACGTTGACTGGAAA |
| [455; 672] | | TLEVEPSDTIENV | GACTATCACTTTGGAAGTGGAGCCTTCCGATA |
| | | KAKIQDKEGIPPD | CTATCGAGAATGTTAAGGCCAAAATCCAAGA |
| | | QQRLAFQGKSLE | TAAGGAAGGGATTCCTCCAGATCAACAACGC |
| | | DGRTLSDYNILKD | CTTGCTTTTCAAGGGAAGAGCCTGGAGGACG |
| | | SKLHPLLRLR | GTCGCACACTGTCTGACTATAACATTCTTAAA |
| | | | GATTCTAAATTGCATCCACTGCTGCGCTTGCG |
| | | | T |
| i53 K63I | K63I | MLIFVKTLTGKTI | ATGTTGATTTTCGTAAAGACGTTGACTGGAAA |
| [456; 673] | | TLEVEPSDTIENV | GACTATCACTTTGGAAGTGGAGCCTTCCGATA |
| | | KAKIQDKEGIPPD | CTATCGAGAATGTTAAGGCCAAAATCCAAGA |
| | | QQRLAFAGKSLE | TAAGGAAGGGATTCCTCCAGATCAACAACGC |
| | | DGRTLSDYNILID | CTTGCTTTTGCCGGGAAGAGCCTGGAGGACG |
| | | SKLHPLLRLR | GTCGCACACTGTCTGACTATAACATTCTTATT |
| | | | GATTCTAAATTGCATCCACTGCTGCGCTTGCG |
| | | | T |
| i53 S65P | S65P | MLIFVKTLTGKTI | ATGTTGATTTTCGTAAAGACGTTGACTGGAAA |
| [457; 674] | | TLEVEPSDTIENV | GACTATCACTTTGGAAGTGGAGCCTTCCGATA |
| | | KAKIQDKEGIPPD | CTATCGAGAATGTTAAGGCCAAAATCCAAGA |
| | | QQRLAFAGKSLE | TAAGGAAGGGATTCCTCCAGATCAACAACGC |
| | | DGRTLSDYNILKD | CTTGCTTTTGCCGGGAAGAGCCTGGAGGACG |
| | | PKLHPLLRLR | GTCGCACACTGTCTGACTATAACATTCTTAAA |
| | | | GATCCTAAATTGCATCCACTGCTGCGCTTGCG |
| | | | T |
| SM1 | M1H | HLIFVKTLTGKTIT | CATTTGATTTTCGTAAAGACGTTGACTGGAAA |
| [458; 675] | | LEVEPSDTIENVK | GACTATCACTTTGGAAGTGGAGCCTTCCGATA |
| | | AKIQDKEGIPPD | CTATCGAGAATGTTAAGGCCAAAATCCAAGA |
| | | QQRLAFAGKSLE | TAAGGAAGGGATTCCTCCAGATCAACAACGC |
| | | DGRTLSDYNILKD | CTTGCTTTTGCCGGGAAGAGCCTGGAGGACG |
| | | SKLHPLLRLR | GTCGCACACTGTCTGACTATAACATTCTTAAA |
| | | | GATTCTAAATTGCATCCACTGCTGCGCTTGCG |
| | | | T |
| SM2 | K6R | MLIFVRTLTGKTI | ATGTTGATTTTCGTACGCACGTTGACTGGAAA |
| [459; 676] | | TLEVEPSDTIENV | GACTATCACTTTGGAAGTGGAGCCTTCCGATA |
| | | KAKIQDKEGIPPD | CTATCGAGAATGTTAAGGCCAAAATCCAAGA |
| | | QQRLAFAGKSLE | TAAGGAAGGGATTCCTCCAGATCAACAACGC |
| | | DGRTLSDYNILKD | CTTGCTTTTGCCGGGAAGAGCCTGGAGGACG |
| | | SKLHPLLRLR | GTCGCACACTGTCTGACTATAACATTCTTAAA |
| | | | GATTCTAAATTGCATCCACTGCTGCGCTTGCG |
| | | | T |
| SM3 | T7M | MLIFVKMLTGKTI | ATGTTGATTTTCGTAAAGATGTTGACTGGAAA |
| [460; 677] | | TLEVEPSDTIENV | GACTATCACTTTGGAAGTGGAGCCTTCCGATA |
| | | KAKIQDKEGIPPD | CTATCGAGAATGTTAAGGCCAAAATCCAAGA |
| | | QQRLAFAGKSLE | TAAGGAAGGGATTCCTCCAGATCAACAACGC |
| | | DGRTLSDYNILKD | CTTGCTTTTGCCGGGAAGAGCCTGGAGGACG |
| | | SKLHPLLRLR | GTCGCACACTGTCTGACTATAACATTCTTAAA |
| | | | GATTCTAAATTGCATCCACTGCTGCGCTTGCG |
| | | | T |
| SM4 | T12M | MLIFVKTLTGKMI | ATGTTGATTTTCGTAAAGACGTTGACTGGAAA |
| [461; 678] | | TLEVEPSDTIENV | GATGATCACTTTGGAAGTGGAGCCTTCCGAT |
| | | KAKIQDKEGIPPD | ACTATCGAGAATGTTAAGGCCAAAATCCAAG |
| | | QQRLAFAGKSLE | ATAAGGAAGGGATTCCTCCAGATCAACAACG |
| | | DGRTLSDYNILKD | CCTTGCTTTTGCCGGGAAGAGCCTGGAGGAC |
| | | SKLHPLLRLR | GGTCGCACACTGTCTGACTATAACATTCTTAA |
| | | | AGATTCTAAATTGCATCCACTGCTGCGCTTGC |
| | | | GT |
| SM5 | T14E | MLIFVKTLTGKTI | ATGTTGATTTTCGTAAAGACGTTGACTGGAAA |
| [462; 679] | | ELEVEPSDTIENV | GACTATCGAGTTGGAAGTGGAGCCTTCCGAT |
| | | KAKIQDKEGIPPD | ACTATCGAGAATGTTAAGGCCAAAATCCAAG |
| | | QQRLAFAGKSLE | ATAAGGAAGGGATTCCTCCAGATCAACAACG |
| | | DGRTLSDYNILKD | CCTTGCTTTTGCCGGGAAGAGCCTGGAGGAC |
| | | SKLHPLLRLR | GGTCGCACACTGTCTGACTATAACATTCTTAA |
| | | | AGATTCTAAATTGCATCCACTGCTGCGCTTGC |
| | | | GT |
| SM6 | E16M | MLIFVKTLTGKTI | ATGTTGATTTTCGTAAAGACGTTGACTGGAAA |
| [463; 680] | | TLMVEPSDTIEN | GACTATCACTTTGATGGTGGAGCCTTCCGATA |
| | | VKAKIQDKEGIPP | CTATCGAGAATGTTAAGGCCAAAATCCAAGA |
| | | DQQRLAFAGKSL | TAAGGAAGGGATTCCTCCAGATCAACAACGC |
| | | EDGRTLSDYNILK | CTTGCTTTTGCCGGGAAGAGCCTGGAGGACG |
| | | DSKLHPLLRLR | GTCGCACACTGTCTGACTATAACATTCTTAAA |
| | | | GATTCTAAATTGCATCCACTGCTGCGCTTGCG |
| | | | T |
| SM7 | E18M | MLIFVKTLTGKTI | ATGTTGATTTTCGTAAAGACGTTGACTGGAAA |
| [464; 681] | | TLEVMPSDTIEN | GACTATCACTTTGGAAGTGATGCCTTCCGATA |
| | | VKAKIQDKEGIPP | CTATCGAGAATGTTAAGGCCAAAATCCAAGA |
| | | DQQRLAFAGKSL | TAAGGAAGGGATTCCTCCAGATCAACAACGC |
| | | EDGRTLSDYNILK | CTTGCTTTTGCCGGGAAGAGCCTGGAGGACG |
| | | DSKLHPLLRLR | GTCGCACACTGTCTGACTATAACATTCTTAAA |
| | | | GATTCTAAATTGCATCCACTGCTGCGCTTGCG |
| | | | T |
| SM8 | N25V | MLIFVKTLTGKTI | ATGTTGATTTTCGTAAAGACGTTGACTGGAAA |
| [465; 682] | | TLEVEPSDTIEVV | GACTATCACTTTGGAAGTGGAGCCTTCCGATA |
| | | KAKIQDKEGIPPD | CTATCGAGGTAGTTAAGGCCAAAATCCAAGA |
| | | QQRLAFAGKSLE | TAAGGAAGGGATTCCTCCAGATCAACAACGC |
| | | DGRTLSDYNILKD | CTTGCTTTTGCCGGGAAGAGCCTGGAGGACG |
| | | SKLHPLLRLR | GTCGCACACTGTCTGACTATAACATTCTTAAA |
| | | | GATTCTAAATTGCATCCACTGCTGCGCTTGCG |
| | | | T |
| SM9 | V26I | MLIFVKTLTGKTI | ATGTTGATTTTCGTAAAGACGTTGACTGGAAA |
| [466; 683] | | TLEVEPSDTIENIK | GACTATCACTTTGGAAGTGGAGCCTTCCGATA |
| | | AKIQDKEGIPPD | CTATCGAGAATATTAAGGCCAAAATCCAAGA |
| | | QQRLAFAGKSLE | TAAGGAAGGGATTCCTCCAGATCAACAACGC |
| | | DGRTLSDYNILKD | CTTGCTTTTGCCGGGAAGAGCCTGGAGGACG |
| | | SKLHPLLRLR | GTCGCACACTGTCTGACTATAACATTCTTAAA |
| | | | GATTCTAAATTGCATCCACTGCTGCGCTTGCG |
| | | | T |
| SM10 | Q31W | MLIFVKTLTGKTI | ATGTTGATTTTCGTAAAGACGTTGACTGGAAA |
| [467; 684] | | TLEVEPSDTIENV | GACTATCACTTTGGAAGTGGAGCCTTCCGATA |
| | | KAKIWDKEGIPP | CTATCGAGAATGTTAAGGCCAAAATCTGGGA |
| | | DQQRLAFAGKSL | TAAGGAAGGGATTCCTCCAGATCAACAACGC |
| | | EDGRTLSDYNILK | CTTGCTTTTGCCGGGAAGAGCCTGGAGGACG |
| | | DSKLHPLLRLR | GTCGCACACTGTCTGACTATAACATTCTTAAA |
| | | | GATTCTAAATTGCATCCACTGCTGCGCTTGCG |
| | | | T |
| SM11 | Q310 | MLIFVKTLTGKTI | ATGTTGATTTTCGTAAAGACGTTGACTGGAAA |
| [468; 685] | | TLEVEPSDTIENV | GACTATCACTTTGGAAGTGGAGCCTTCCGATA |
| | | KAKICDKEGIPPD | CTATCGAGAATGTTAAGGCCAAAATCTGCGA |
| | | QQRLAFAGKSLE | TAAGGAAGGGATTCCTCCAGATCAACAACGC |
| | | DGRTLSDYNILKD | CTTGCTTTTGCCGGGAAGAGCCTGGAGGACG |
| | | SKLHPLLRLR | GTCGCACACTGTCTGACTATAACATTCTTAAA |
| | | | GATTCTAAATTGCATCCACTGCTGCGCTTGCG |
| | | | T |
| SM12 | Q31F | MLIFVKTLTGKTI | ATGTTGATTTTCGTAAAGACGTTGACTGGAAA |
| [469; 686] | | TLEVEPSDTIENV | GACTATCACTTTGGAAGTGGAGCCTTCCGATA |
| | | KAKIFDKEGIPPD | CTATCGAGAATGTTAAGGCCAAAATCTTCGAT |
| | | QQRLAFAGKSLE | AAGGAAGGGATTCCTCCAGATCAACAACGCC |
| | | DGRTLSDYNILKD | TTGCTTTTGCCGGGAAGAGCCTGGAGGACGG |
| | | SKLHPLLRLR | TCGCACACTGTCTGACTATAACATTCTTAAAG |
| | | | ATTCTAAATTGCATCCACTGCTGCGCTTGCGT |
| SM13 | K33S | MLIFVKTLTGKTI | ATGTTGATTTTCGTAAAGACGTTGACTGGAAA |
| [470; 687] | | TLEVEPSDTIENV | GACTATCACTTTGGAAGTGGAGCCTTCCGATA |
| | | KAKIQDSEGIPPD | CTATCGAGAATGTTAAGGCCAAAATCCAAGA |
| | | QQRLAFAGKSLE | TTCTGAAGGGATTCCTCCAGATCAACAACGCC |
| | | DGRTLSDYNILKD | TTGCTTTTGCCGGGAAGAGCCTGGAGGACGG |
| | | SKLHPLLRLR | TCGCACACTGTCTGACTATAACATTCTTAAAG |
| | | | ATTCTAAATTGCATCCACTGCTGCGCTTGCGT |
| SM14 | K33H | MLIFVKTLTGKTI | ATGTTGATTTTCGTAAAGACGTTGACTGGAAA |
| [471; 688] | | TLEVEPSDTIENV | GACTATCACTTTGGAAGTGGAGCCTTCCGATA |
| | | KAKIQDHEGIPP | CTATCGAGAATGTTAAGGCCAAAATCCAAGA |
| | | DQQRLAFAGKSL | TCATGAAGGGATTCCTCCAGATCAACAACGC |
| | | EDGRTLSDYNILK | CTTGCTTTTGCCGGGAAGAGCCTGGAGGACG |
| | | DSKLHPLLRLR | GTCGCACACTGTCTGACTATAACATTCTTAAA |
| | | | GATTCTAAATTGCATCCACTGCTGCGCTTGCG |
| | | | T |
| SM15 | K33A | MLIFVKTLTGKTI | ATGTTGATTTTCGTAAAGACGTTGACTGGAAA |
| [472; 689] | | TLEVEPSDTIENV | GACTATCACTTTGGAAGTGGAGCCTTCCGATA |
| | | KAKIQDAEGIPPD | CTATCGAGAATGTTAAGGCCAAAATCCAAGA |
| | | QQRLAFAGKSLE | TGCCGAAGGGATTCCTCCAGATCAACAACGC |
| | | DGRTLSDYNILKD | CTTGCTTTTGCCGGGAAGAGCCTGGAGGACG |
| | | SKLHPLLRLR | GTCGCACACTGTCTGACTATAACATTCTTAAA |
| | | | GATTCTAAATTGCATCCACTGCTGCGCTTGCG |
| | | | T |
| SM16 | P38L | MLIFVKTLTGKTI | ATGTTGATTTTCGTAAAGACGTTGACTGGAAA |
| [473; 690] | | TLEVEPSDTIENV | GACTATCACTTTGGAAGTGGAGCCTTCCGATA |
| | | KAKIQDKEGIPLD | CTATCGAGAATGTTAAGGCCAAAATCCAAGA |
| | | QQRLAFAGKSLE | TAAGGAAGGGATTCCTTTGGATCAACAACGC |
| | | DGRTLSDYNILKD | CTTGCTTTTGCCGGGAAGAGCCTGGAGGACG |
| | | SKLHPLLRLR | GTCGCACACTGTCTGACTATAACATTCTTAAA |
| | | | GATTCTAAATTGCATCCACTGCTGCGCTTGCG |
| | | | T |
| SM17 | R42W | MLIFVKTLTGKTI | ATGTTGATTTTCGTAAAGACGTTGACTGGAAA |
| [474; 691] | | TLEVEPSDTIENV | GACTATCACTTTGGAAGTGGAGCCTTCCGATA |
| | | KAKIQDKEGIPPD | CTATCGAGAATGTTAAGGCCAAAATCCAAGA |
| | | QQWLAFAGKSL | TAAGGAAGGGATTCCTCCAGATCAACAATGG |
| | | EDGRTLSDYNILK | CTTGCTTTTGCCGGGAAGAGCCTGGAGGACG |
| | | DSKLHPLLRLR | GTCGCACACTGTCTGACTATAACATTCTTAAA |
| | | | GATTCTAAATTGCATCCACTGCTGCGCTTGCG |
| | | | T |
| SM18 | A44T | MLIFVKTLTGKTI | ATGTTGATTTTCGTAAAGACGTTGACTGGAAA |
| [475; 692] | | TLEVEPSDTIENV | GACTATCACTTTGGAAGTGGAGCCTTCCGATA |
| | | KAKIQDKEGIPPD | CTATCGAGAATGTTAAGGCCAAAATCCAAGA |
| | | QQRLTFAGKSLE | TAAGGAAGGGATTCCTCCAGATCAACAACGC |
| | | DGRTLSDYNILKD | CTTACTTTTGCCGGGAAGAGCCTGGAGGACG |
| | | SKLHPLLRLR | GTCGCACACTGTCTGACTATAACATTCTTAAA |
| | | | GATTCTAAATTGCATCCACTGCTGCGCTTGCG |
| | | | T |
| SM19 | A46Q | MLIFVKTLTGKTI | ATGTTGATTTTCGTAAAGACGTTGACTGGAAA |
| [476; 693] | | TLEVEPSDTIENV | GACTATCACTTTGGAAGTGGAGCCTTCCGATA |
| | | KAKIQDKEGIPPD | CTATCGAGAATGTTAAGGCCAAAATCCAAGA |
| | | QQRLAFQGKSLE | TAAGGAAGGGATTCCTCCAGATCAACAACGC |
| | | DGRTLSDYNILKD | CTTGCTTTTCAAGGGAAGAGCCTGGAGGACG |
| | | SKLHPLLRLR | GTCGCACACTGTCTGACTATAACATTCTTAAA |
| | | | GATTCTAAATTGCATCCACTGCTGCGCTTGCG |
| | | | T |
| SM20 | K48T | MLIFVKTLTGKTI | ATGTTGATTTTCGTAAAGACGTTGACTGGAAA |
| [477; 694] | | TLEVEPSDTIENV | GACTATCACTTTGGAAGTGGAGCCTTCCGATA |
| | | KAKIQDKEGIPPD | CTATCGAGAATGTTAAGGCCAAAATCCAAGA |
| | | QQRLAFAGTSLE | TAAGGAAGGGATTCCTCCAGATCAACAACGC |
| | | DGRTLSDYNILKD | CTTGCTTTTGCCGGGACTAGCCTGGAGGACG |
| | | SKLHPLLRLR | GTCGCACACTGTCTGACTATAACATTCTTAAA |
| | | | GATTCTAAATTGCATCCACTGCTGCGCTTGCG |
| | | | T |
| SM21 | R54Y | MLIFVKTLTGKTI | ATGTTGATTTTCGTAAAGACGTTGACTGGAAA |
| [478; 695] | | TLEVEPSDTIENV | GACTATCACTTTGGAAGTGGAGCCTTCCGATA |
| | | KAKIQDKEGIPPD | CTATCGAGAATGTTAAGGCCAAAATCCAAGA |
| | | QQRLAFAGKSLE | TAAGGAAGGGATTCCTCCAGATCAACAACGC |
| | | DGYTLSDYNILKD | CTTGCTTTTGCCGGGAAGAGCCTGGAGGACG |
| | | SKLHPLLRLR | GTTATACACTGTCTGACTATAACATTCTTAAA |
| | | | GATTCTAAATTGCATCCACTGCTGCGCTTGCG |
| | | | T |
| SM22 | S57G | MLIFVKTLTGKTI | ATGTTGATTTTCGTAAAGACGTTGACTGGAAA |
| [479; 696] | | TLEVEPSDTIENV | GACTATCACTTTGGAAGTGGAGCCTTCCGATA |
| | | KAKIQDKEGIPPD | CTATCGAGAATGTTAAGGCCAAAATCCAAGA |
| | | QQRLAFAGKSLE | TAAGGAAGGGATTCCTCCAGATCAACAACGC |
| | | DGRTLGDYNILK | CTTGCTTTTGCCGGGAAGAGCCTGGAGGACG |
| | | DSKLHPLLRLR | GTCGCACACTGGGGGACTATAACATTCTTAA |
| | | | AGATTCTAAATTGCATCCACTGCTGCGCTTGC |
| | | | GT |
| SM23 | K63I | MLIFVKTLTGKTI | ATGTTGATTTTCGTAAAGACGTTGACTGGAAA |
| [480; 697] | | TLEVEPSDTIENV | GACTATCACTTTGGAAGTGGAGCCTTCCGATA |
| | | KAKIQDKEGIPPD | CTATCGAGAATGTTAAGGCCAAAATCCAAGA |
| | | QQRLAFAGKSLE | TAAGGAAGGGATTCCTCCAGATCAACAACGC |
| | | DGRTLSDYNILID | CTTGCTTTTGCCGGGAAGAGCCTGGAGGACG |
| | | SKLHPLLRLR | GTCGCACACTGTCTGACTATAACATTCTTATT |
| | | | GATTCTAAATTGCATCCACTGCTGCGCTTGCG |
| | | | T |
| SM24 | S65P | MLIFVKTLTGKTI | ATGTTGATTTTCGTAAAGACGTTGACTGGAAA |
| [481; 698] | | TLEVEPSDTIENV | GACTATCACTTTGGAAGTGGAGCCTTCCGATA |
| | | KAKIQDKEGIPPD | CTATCGAGAATGTTAAGGCCAAAATCCAAGA |
| | | QQRLAFAGKSLE | TAAGGAAGGGATTCCTCCAGATCAACAACGC |
| | | DGRTLSDYNILKD | CTTGCTTTTGCCGGGAAGAGCCTGGAGGACG |
| | | PKLHPLLRLR | GTCGCACACTGTCTGACTATAACATTCTTAAA |
| | | | GATCCTAAATTGCATCCACTGCTGCGCTTGCG |
| | | | T |
| CM1tf | K6R, T7M, | MLIFVRMLTGK | ATGTTGATTTTCGTACGCATGTTGACTGGAAA |
| [482; 699] | T12M, T14E, | MIELEVEPSDTIE | GATGATCGAGTTGGAAGTGGAGCCTTCCGAT |
| | K33H, A46Q, | NVKAKIQDHEGI | ACTATCGAGAATGTTAAGGCCAAAATCCAAG |
| | S65P, L67K, | PPDQQRLAFQG | ATCATGAAGGGATTCCTCCAGATCAACAACG |
| | H68M | KSLEDGRTLSDY | CCTTGCTTTTCAAGGGAAGAGCCTGGAGGAC |
| | | NILKDPKKMPLLR | GGTCGCACACTGTCTGACTATAACATTCTTAA |
| | | LR | AGATCCTAAAAAGATGCCACTGCTGCGCTTG |
| | | | CGT |
| CM7 | K6R, K33H, | MLIFVRTLTGKTI | ATGTTGATTTTCGTACGCACGTTGACTGGAAA |
| [483; 700] | A46Q, S65P | TLEVEPSDTIENV | GACTATCACTTTGGAAGTGGAGCCTTCCGATA |
| | | KAKIQDHEGIPP | CTATCGAGAATGTTAAGGCCAAAATCCAAGA |
| | | DQQRLAFQGKSL | TCATGAAGGGATTCCTCCAGATCAACAACGC |
| | | EDGRTLSDYNILK | CTTGCTTTTCAAGGGAAGAGCCTGGAGGACG |
| | | DPKLHPLLRLR | GTCGCACACTGTCTGACTATAACATTCTTAAA |
| | | | GATCCTAAATTGCATCCACTGCTGCGCTTGCG |
| | | | T |
| CM13 | T7M, T14E, | MLIFVKMLTGKTI | ATGTTGATTTTCGTAAAGATGTTGACTGGAAA |
| [484; 701] | A46Q, L67K | ELEVEPSDTIENV | GACTATCGAGTTGGAAGTGGAGCCTTCCGAT |
| | | KAKIQDKEGIPPD | ACTATCGAGAATGTTAAGGCCAAAATCCAAG |
| | | QQRLAFQGKSLE | ATAAGGAAGGGATTCCTCCAGATCAACAACG |
| | | DGRTLSDYNILKD | CCTTGCTTTTCAAGGGAAGAGCCTGGAGGAC |
| | | SKKHPLLRLR | GGTCGCACACTGTCTGACTATAACATTCTTAA |
| | | | AGATTCTAAAAAGCATCCACTGCTGCGCTTGC |
| | | | GT |
| CM26 | T12M, K33H, | MLIFVKTLTGKMI | ATGTTGATTTTCGTAAAGACGTTGACTGGAAA |
| [485; 702] | A46q, H68M | TLEVEPSDTIENV | GATGATCACTTTGGAAGTGGAGCCTTCCGAT |
| | | KAKIQDHEGIPP | ACTATCGAGAATGTTAAGGCCAAAATCCAAG |
| | | DQQRLAFQGKSL | ATCATGAAGGGATTCCTCCAGATCAACAACG |
| | | EDGRTLSDYNILK | CCTTGCTTTTCAAGGGAAGAGCCTGGAGGAC |
| | | DSKLMPLLRLR | GGTCGCACACTGTCTGACTATAACATTCTTAA |
| | | | AGATTCTAAATTGATGCCACTGCTGCGCTTGC |
| | | | GT |
| CM44 | T7M, T12M, | MLIFVKMLTGK | ATGTTGATTTTCGTAAAGATGTTGACTGGAAA |
| [486; 703] | T14E, K33H, | MIELEVEPSDTIE | GATGATCGAGTTGGAAGTGGAGCCTTCCGAT |
| | A46Q, S65P, | NVKAKIQDHEGI | ACTATCGAGAATGTTAAGGCCAAAATCCAAG |
| | L67K, H68M | PPDQQRLAFQG | ATCATGAAGGGATTCCTCCAGATCAACAACG |
| | | KSLEDGRTLSDY | CCTTGCTTTTCAAGGGAAGAGCCTGGAGGAC |
| | | NILKDPKKMPLLR | GGTCGCACACTGTCTGACTATAACATTCTTAA |
| | | LR | AGATCCTAAAAAGATGCCACTGCTGCGCTTG |
| | | | CGT |
| CM45 | K6R, T12M, | MLIFVRTLTGKMI | ATGTTGATTTTCGTACGCACGTTGACTGGAAA |
| [487; 704] | T14E, K33H, | ELEVEPSDTIENV | GATGATCGAGTTGGAAGTGGAGCCTTCCGAT |
| | A46Q, S65P, | KAKIQDHEGIPP | ACTATCGAGAATGTTAAGGCCAAAATCCAAG |
| | L67K, H68M | DQQRLAFQGKSL | ATCATGAAGGGATTCCTCCAGATCAACAACG |
| | | EDGRTLSDYNILK | CCTTGCTTTTCAAGGGAAGAGCCTGGAGGAC |
| | | DPKKMPLLRLR | GGTCGCACACTGTCTGACTATAACATTCTTAA |
| | | | AGATCCTAAAAAGATGCCACTGCTGCGCTTG |
| | | | CGT |
| CM46 | K6R, T7M, | MLIFVRMLTGKTI | ATGTTGATTTTCGTACGCATGTTGACTGGAAA |
| [488; 705] | T14E, K33H, | ELEVEPSDTIENV | GACTATCGAGTTGGAAGTGGAGCCTTCCGAT |
| | A46Q, S65P, | KAKIQDHEGIPP | ACTATCGAGAATGTTAAGGCCAAAATCCAAG |
| | L67K, H68M | DQQRLAFQGKSL | ATCATGAAGGGATTCCTCCAGATCAACAACG |
| | | EDGRTLSDYNILK | CCTTGCTTTTCAAGGGAAGAGCCTGGAGGAC |
| | | DPKKMPLLRLR | GGTCGCACACTGTCTGACTATAACATTCTTAA |
| | | | AGATCCTAAAAAGATGCCACTGCTGCGCTTG |
| | | | CGT |
| CM47 | K6R, T7M, | MLIFVRMLTGK | ATGTTGATTTTCGTACGCATGTTGACTGGAAA |
| [489; 706] | T12M, K33H, | MITLEVEPSDTIE | GATGATCACTTTGGAAGTGGAGCCTTCCGAT |
| | A46Q, S65P, | NVKAKIQDHEGI | ACTATCGAGAATGTTAAGGCCAAAATCCAAG |
| | L67K, H68M | PPDQQRLAFQG | ATCATGAAGGGATTCCTCCAGATCAACAACG |
| | | KSLEDGRTLSDY | CCTTGCTTTTCAAGGGAAGAGCCTGGAGGAC |
| | | NILKDPKKMPLLR | GGTCGCACACTGTCTGACTATAACATTCTTAA |
| | | LR | AGATCCTAAAAAGATGCCACTGCTGCGCTTG |
| | | | CGT |
| CM48 | K6R, T7M, | MLIFVRMLTGK | ATGTTGATTTTCGTACGCATGTTGACTGGAAA |
| [490; 707] | T12M, T14E, | MIELEVEPSDTIE | GATGATCGAGTTGGAAGTGGAGCCTTCCGAT |
| | A46Q, S65P, | NVKAKIQDKEGIP | ACTATCGAGAATGTTAAGGCCAAAATCCAAG |
| | L67K, H68M | PDQQRLAFQGKS | ATAAGGAAGGGATTCCTCCAGATCAACAACG |
| | | LEDGRTLSDYNIL | CCTTGCTTTTCAAGGGAAGAGCCTGGAGGAC |
| | | KDPKKMPLLRLR | GGTCGCACACTGTCTGACTATAACATTCTTAA |
| | | | AGATCCTAAAAAGATGCCACTGCTGCGCTTG |
| | | | CGT |
| CM49 | K6R, T7M, | MLIFVRMLTGK | ATGTTGATTTTCGTACGCATGTTGACTGGAAA |
| [491; 708] | T12M, T14E, | MIELEVEPSDTIE | GATGATCGAGTTGGAAGTGGAGCCTTCCGAT |
| | K33H, S65P, | NVKAKIQDHEGI | ACTATCGAGAATGTTAAGGCCAAAATCCAAG |
| | L67K, H68M | PPDQQRLAFAGK | ATCATGAAGGGATTCCTCCAGATCAACAACG |
| | | SLEDGRTLSDYNI | CCTTGCTTTTGCCGGGAAGAGCCTGGAGGAC |
| | | LKDPKKMPLLRL | GGTCGCACACTGTCTGACTATAACATTCTTAA |
| | | R | AGATCCTAAAAAGATGCCACTGCTGCGCTTG |
| | | | CGT |
| CM50 | K6R, T7M, | MLIFVRMLTGK | ATGTTGATTTTCGTACGCATGTTGACTGGAAA |
| [492; 709] | T12M, T14E, | MIELEVEPSDTIE | GATGATCGAGTTGGAAGTGGAGCCTTCCGAT |
| | K33H, A46Q, | NVKAKIQDHEGI | ACTATCGAGAATGTTAAGGCCAAAATCCAAG |
| | L67K, H68M | PPDQQRLAFQG | ATCATGAAGGGATTCCTCCAGATCAACAACG |
| | | KSLEDGRTLSDY | CCTTGCTTTTCAAGGGAAGAGCCTGGAGGAC |
| | | NILKDSKKMPLLR | GGTCGCACACTGTCTGACTATAACATTCTTAA |
| | | LR | AGATTCTAAAAAGATGCCACTGCTGCGCTTGC |
| | | | GT |
| CM51 | K6R, T7M, | MLIFVRMLTGK | ATGTTGATTTTCGTACGCATGTTGACTGGAAA |
| [493; 710] | T12M, T14E, | MIELEVEPSDTIE | GATGATCGAGTTGGAAGTGGAGCCTTCCGAT |
| | K33H, A46Q, | NVKAKIQDHEGI | ACTATCGAGAATGTTAAGGCCAAAATCCAAG |
| | S65P, H68M | PPDQQRLAFQG | ATCATGAAGGGATTCCTCCAGATCAACAACG |
| | | KSLEDGRTLSDY | CCTTGCTTTTCAAGGGAAGAGCCTGGAGGAC |
| | | NILKDPKLMPLLR | GGTCGCACACTGTCTGACTATAACATTCTTAA |
| | | LR | AGATCCTAAATTGATGCCACTGCTGCGCTTGC |
| | | | GT |
| CM52 | K6R, T7M, | MLIFVRMLTGK | ATGTTGATTTTCGTACGCATGTTGACTGGAAA |
| [494; 711] | T12M, T14E, | MIELEVEPSDTIE | GATGATCGAGTTGGAAGTGGAGCCTTCCGAT |
| | K33H, A46Q, | NVKAKIQDHEGI | ACTATCGAGAATGTTAAGGCCAAAATCCAAG |
| | S65P, L67K | PPDQQRLAFQG | ATCATGAAGGGATTCCTCCAGATCAACAACG |
| | | KSLEDGRTLSDY | CCTTGCTTTTCAAGGGAAGAGCCTGGAGGAC |
| | | NILKDPKKHPLLR | GGTCGCACACTGTCTGACTATAACATTCTTAA |
| | | LR | AGATCCTAAAAAGCATCCACTGCTGCGCTTGC |
| | | | GT |
| CM62 | K6R, T7M, | HLIFVRMLTGKM | CATTTGATTTTCGTACGCATGTTGACTGGAAA |
| [495; 712] | T12M, T14E, | IELEVEPSDTIENV | GATGATCGAGTTGGAAGTGGAGCCTTCCGAT |
| | K33H, A46Q, | KAKIQDHEGIPP | ACTATCGAGAATGTTAAGGCCAAAATCCAAG |
| | S65P, L67K, | DQQRLAFQGKSL | ATCATGAAGGGATTCCTCCAGATCAACAACG |
| | H68M, MIH | EDGRTLSDYNILK | CCTTGCTTTTCAAGGGAAGAGCCTGGAGGAC |
| | | DPKKMPLLRLR | GGTCGCACACTGTCTGACTATAACATTCTTAA |
| | | | AGATCCTAAAAAGATGCCACTGCTGCGCTTG |
| | | | CGT |
| CM63 | K6R, T7M, | YLIFVRMLTGKMI | TATTTGATTTTCGTACGCATGTTGACTGGAAA |
| [496; 713] | T12M, T14E, | ELEVEPSDTIENV | GATGATCGAGTTGGAAGTGGAGCCTTCCGAT |
| | K33H, A46Q, | KAKIQDHEGIPP | ACTATCGAGAATGTTAAGGCCAAAATCCAAG |
| | S65P, L67K, | DQQRLAFQGKSL | ATCATGAAGGGATTCCTCCAGATCAACAACG |
| | H68M, M1Y | EDGRTLSDYNILK | CCTTGCTTTTCAAGGGAAGAGCCTGGAGGAC |
| | | DPKKMPLLRLR | GGTCGCACACTGTCTGACTATAACATTCTTAA |
| | | | AGATCCTAAAAAGATGCCACTGCTGCGCTTG |
| | | | CGT |
| CM64 | K6R, T7M, | MLIFVRMLTGK | ATGTTGATTTTCGTACGCATGTTGACTGGAAA |
| [497; 714] | T12M, T14H, | MIHLEVEPSDTIE | GATGATCCATTTGGAAGTGGAGCCTTCCGAT |
| | K33H, A46Q, | NVKAKIQDHEGI | ACTATCGAGAATGTTAAGGCCAAAATCCAAG |
| | S65P, L67K, | PPDQQRLAFQG | ATCATGAAGGGATTCCTCCAGATCAACAACG |
| | H68M | KSLEDGRTLSDY | CCTTGCTTTTCAAGGGAAGAGCCTGGAGGAC |
| | | NILKDPKKMPLLR | GGTCGCACACTGTCTGACTATAACATTCTTAA |
| | | LR | AGATCCTAAAAAGATGCCACTGCTGCGCTTG |
| | | | CGT |
| CM65 | K6R, T7M, | MLIFVRMLTGK | ATGTTGATTTTCGTACGCATGTTGACTGGAAA |
| [498; 715] | T12M, T14D, | MIDLEVEPSDTIE | GATGATCGATTTGGAAGTGGAGCCTTCCGAT |
| | K33H, A46Q, | NVKAKIQDHEGI | ACTATCGAGAATGTTAAGGCCAAAATCCAAG |
| | S65P, L67K, | PPDQQRLAFQG | ATCATGAAGGGATTCCTCCAGATCAACAACG |
| | H68M | KSLEDGRTLSDY | CCTTGCTTTTCAAGGGAAGAGCCTGGAGGAC |
| | | NILKDPKKMPLLR | GGTCGCACACTGTCTGACTATAACATTCTTAA |
| | | LR | AGATCCTAAAAAGATGCCACTGCTGCGCTTG |
| | | | CGT |
| CM66 | K6R, T7M, | MLIFVRMLTGK | ATGTTGATTTTCGTACGCATGTTGACTGGAAA |
| [499; 716] | T12M, T14E, | MIELMVEPSDTIE | GATGATCGAGTTGATGGTGGAGCCTTCCGAT |
| | K33H, A46Q, | NVKAKIQDHEGI | ACTATCGAGAATGTTAAGGCCAAAATCCAAG |
| | S65P, L67K, | PPDQQRLAFQG | ATCATGAAGGGATTCCTCCAGATCAACAACG |
| | H68M, E16M | KSLEDGRTLSDY | CCTTGCTTTTCAAGGGAAGAGCCTGGAGGAC |
| | | NILKDPKKMPLLR | GGTCGCACACTGTCTGACTATAACATTCTTAA |
| | | LR | AGATCCTAAAAAGATGCCACTGCTGCGCTTG |
| | | | CGT |
| CM67 | K6R, T7M, | MLIFVRMLTGK | ATGTTGATTTTCGTACGCATGTTGACTGGAAA |
| [500; 717] | T12M, T14E, | MIELTVEPSDTIE | GATGATCGAGTTGACTGTGGAGCCTTCCGAT |
| | K33H, A46Q, | NVKAKIQDHEGI | ACTATCGAGAATGTTAAGGCCAAAATCCAAG |
| | S65P, L67K, | PPDQQRLAFQG | ATCATGAAGGGATTCCTCCAGATCAACAACG |
| | H68M, E16T | KSLEDGRTLSDY | CCTTGCTTTTCAAGGGAAGAGCCTGGAGGAC |
| | | NILKDPKKMPLLR | GGTCGCACACTGTCTGACTATAACATTCTTAA |
| | | LR | AGATCCTAAAAAGATGCCACTGCTGCGCTTG |
| | | | CGT |
| CM68 | K6R, T7M, | MLIFVRMLTGK | ATGTTGATTTTCGTACGCATGTTGACTGGAAA |
| [501; 718] | T12M, T14E, | MIELEVMPSDTIE | GATGATCGAGTTGGAAGTGATGCCTTCCGAT |
| | K33H, A46Q, | NVKAKIQDHEGI | ACTATCGAGAATGTTAAGGCCAAAATCCAAG |
| | S65P, L67K, | PPDQQRLAFQG | ATCATGAAGGGATTCCTCCAGATCAACAACG |
| | H68M, E18M | KSLEDGRTLSDY | CCTTGCTTTTCAAGGGAAGAGCCTGGAGGAC |
| | | NILKDPKKMPLLR | GGTCGCACACTGTCTGACTATAACATTCTTAA |
| | | LR | AGATCCTAAAAAGATGCCACTGCTGCGCTTG |
| | | | CGT |
| CM69 | K6R, T7M, | MLIFVRMLTGK | ATGTTGATTTTCGTACGCATGTTGACTGGAAA |
| [502; 719] | T12M, T14E, | MIELEVYPSDTIE | GATGATCGAGTTGGAAGTGTATCCTTCCGAT |
| | K33H, A46Q, | NVKAKIQDHEGI | ACTATCGAGAATGTTAAGGCCAAAATCCAAG |
| | S65P, L67K, | PPDQQRLAFQG | ATCATGAAGGGATTCCTCCAGATCAACAACG |
| | H68M, E18Y | KSLEDGRTLSDY | CCTTGCTTTTCAAGGGAAGAGCCTGGAGGAC |
| | | NILKDPKKMPLLR | GGTCGCACACTGTCTGACTATAACATTCTTAA |
| | | LR | AGATCCTAAAAAGATGCCACTGCTGCGCTTG |
| | | | CGT |
| CM70 | K6R, T7M, | MLIFVRMLTGK | ATGTTGATTTTCGTACGCATGTTGACTGGAAA |
| [503; 720] | T12M, T14E, | MIELEVLPSDTIE | GATGATCGAGTTGGAAGTGTTGCCTTCCGAT |
| | K33H, A46Q, | NVKAKIQDHEGI | ACTATCGAGAATGTTAAGGCCAAAATCCAAG |
| | S65P, L67K, | PPDQQRLAFQG | ATCATGAAGGGATTCCTCCAGATCAACAACG |
| | H68M, E18L | KSLEDGRTLSDY | CCTTGCTTTTCAAGGGAAGAGCCTGGAGGAC |
| | | NILKDPKKMPLLR | GGTCGCACACTGTCTGACTATAACATTCTTAA |
| | | LR | AGATCCTAAAAAGATGCCACTGCTGCGCTTG |
| | | | CGT |
| CM71 | K6R, T7M, | MLIFVRMLTGK | ATGTTGATTTTCGTACGCATGTTGACTGGAAA |
| [504; 721] | T12M, T14E, | MIELEVFPSDTIE | GATGATCGAGTTGGAAGTGTTCCCTTCCGATA |
| | K33H, A46Q, | NVKAKIQDHEGI | CTATCGAGAATGTTAAGGCCAAAATCCAAGA |
| | S65P, L67K, | PPDQQRLAFQG | TCATGAAGGGATTCCTCCAGATCAACAACGC |
| | H68M, E18F | KSLEDGRTLSDY | CTTGCTTTTCAAGGGAAGAGCCTGGAGGACG |
| | | NILKDPKKMPLLR | GTCGCACACTGTCTGACTATAACATTCTTAAA |
| | | LR | GATCCTAAAAAGATGCCACTGCTGCGCTTGC |
| | | | GT |
| CM72 | K6R, T7M, | MLIFVRMLTGK | ATGTTGATTTTCGTACGCATGTTGACTGGAAA |
| [505; 722] | T12M, T14E, | MIELEVEPSDTIE | GATGATCGAGTTGGAAGTGGAGCCTTCCGAT |
| | K33H, A46Q, | VVKAKIQDHEGI | ACTATCGAGGTAGTTAAGGCCAAAATCCAAG |
| | S65P, L67K, | PPDQQRLAFQG | ATCATGAAGGGATTCCTCCAGATCAACAACG |
| | H68M, N25V | KSLEDGRTLSDY | CCTTGCTTTTCAAGGGAAGAGCCTGGAGGAC |
| | | NILKDPKKMPLLR | GGTCGCACACTGTCTGACTATAACATTCTTAA |
| | | LR | AGATCCTAAAAAGATGCCACTGCTGCGCTTG |
| | | | CGT |
| CM73 | K6R, T7M, | MLIFVRMLTGK | ATGTTGATTTTCGTACGCATGTTGACTGGAAA |
| [506; 723] | T12M, T14E, | MIELEVEPSDTIE | GATGATCGAGTTGGAAGTGGAGCCTTCCGAT |
| | K33H, A46Q, | EVKAKIQDHEGIP | ACTATCGAGGAGGTTAAGGCCAAAATCCAAG |
| | S65P, L67K, | PDQQRLAFQGKS | ATCATGAAGGGATTCCTCCAGATCAACAACG |
| | H68M, N25E | LEDGRTLSDYNIL | CCTTGCTTTTCAAGGGAAGAGCCTGGAGGAC |
| | | KDPKKMPLLRLR | GGTCGCACACTGTCTGACTATAACATTCTTAA |
| | | | AGATCCTAAAAAGATGCCACTGCTGCGCTTG |
| | | | CGT |
| CM74 | K6R, T7M, | MLIFVRMLTGK | ATGTTGATTTTCGTACGCATGTTGACTGGAAA |
| [507; 724] | T12M, T14E, | MIELEVEPSDTIE | GATGATCGAGTTGGAAGTGGAGCCTTCCGAT |
| | K33H, A46Q, | NIKAKIQDHEGIP | ACTATCGAGAATATTAAGGCCAAAATCCAAG |
| | S65P, L67K, | PDQQRLAFQGKS | ATCATGAAGGGATTCCTCCAGATCAACAACG |
| | H68M, V26I | LEDGRTLSDYNIL | CCTTGCTTTTCAAGGGAAGAGCCTGGAGGAC |
| | | KDPKKMPLLRLR | GGTCGCACACTGTCTGACTATAACATTCTTAA |
| | | | AGATCCTAAAAAGATGCCACTGCTGCGCTTG |
| | | | CGT |
| CM75 | K6R, T7M, | MLIFVRMLTGK | ATGTTGATTTTCGTACGCATGTTGACTGGAAA |
| [508; 725] | T12M, T14E, | MIELEVEPSDTIE | GATGATCGAGTTGGAAGTGGAGCCTTCCGAT |
| | K33H, A46Q, | NVKAKICDHEGIP | ACTATCGAGAATGTTAAGGCCAAAATCTGCG |
| | S65P, L67K, | PDQQRLAFQGKS | ATCATGAAGGGATTCCTCCAGATCAACAACG |
| | H68M, Q31C | LEDGRTLSDYNIL | CCTTGCTTTTCAAGGGAAGAGCCTGGAGGAC |
| | | KDPKKMPLLRLR | GGTCGCACACTGTCTGACTATAACATTCTTAA |
| | | | AGATCCTAAAAAGATGCCACTGCTGCGCTTG |
| | | | CGT |
| CM76 | K6R, T7M, | MLIFVRMLTGK | ATGTTGATTTTCGTACGCATGTTGACTGGAAA |
| [509; 726] | T12M, T14E, | MIELEVEPSDTIE | GATGATCGAGTTGGAAGTGGAGCCTTCCGAT |
| | K33H, A46Q, | NVKAKIWDHEGI | ACTATCGAGAATGTTAAGGCCAAAATCTGGG |
| | S65P, L67K, | PPDQQRLAFQG | ATCATGAAGGGATTCCTCCAGATCAACAACG |
| | H68M, Q31W | KSLEDGRTLSDY | CCTTGCTTTTCAAGGGAAGAGCCTGGAGGAC |
| | | NILKDPKKMPLLR | GGTCGCACACTGTCTGACTATAACATTCTTAA |
| | | LR | AGATCCTAAAAAGATGCCACTGCTGCGCTTG |
| | | | CGT |
| CM77 | K6R, T7M, | MLIFVRMLTGK | ATGTTGATTTTCGTACGCATGTTGACTGGAAA |
| [510; 727] | T12M, T14E, | MIELEVEPSDTIE | GATGATCGAGTTGGAAGTGGAGCCTTCCGAT |
| | K33H, A46Q, | NVKAKIFDHEGIP | ACTATCGAGAATGTTAAGGCCAAAATCTTCG |
| | S65P, L67K, | PDQQRLAFQGKS | ATCATGAAGGGATTCCTCCAGATCAACAACG |
| | H68M, Q31F | LEDGRTLSDYNIL | CCTTGCTTTTCAAGGGAAGAGCCTGGAGGAC |
| | | KDPKKMPLLRLR | GGTCGCACACTGTCTGACTATAACATTCTTAA |
| | | | AGATCCTAAAAAGATGCCACTGCTGCGCTTG |
| | | | CGT |
| CM78 | K6R, T7M, | MLIFVRMLTGK | ATGTTGATTTTCGTACGCATGTTGACTGGAAA |
| [511; 728] | T12M, T14E, | MIELEVEPSDTIE | GATGATCGAGTTGGAAGTGGAGCCTTCCGAT |
| | K33H, A46Q, | NVKAKIQAHEGI | ACTATCGAGAATGTTAAGGCCAAAATCCAAG |
| | S65P, L67K, | PPDQQRLAFQG | CCCATGAAGGGATTCCTCCAGATCAACAACG |
| | H68M, D32A | KSLEDGRTLSDY | CCTTGCTTTTCAAGGGAAGAGCCTGGAGGAC |
| | | NILKDPKKMPLLR | GGTCGCACACTGTCTGACTATAACATTCTTAA |
| | | LR | AGATCCTAAAAAGATGCCACTGCTGCGCTTG |
| | | | CGT |
| CM79 | K6R, T7M, | MLIFVRMLTGK | ATGTTGATTTTCGTACGCATGTTGACTGGAAA |
| [512; 729] | T12M, T14E, | MIELEVEPSDTIE | GATGATCGAGTTGGAAGTGGAGCCTTCCGAT |
| | K33S, A46Q, | NVKAKIQDSEGIP | ACTATCGAGAATGTTAAGGCCAAAATCCAAG |
| | S65P, L67K, | PDQQRLAFQGKS | ATTCTGAAGGGATTCCTCCAGATCAACAACGC |
| | H68M | LEDGRTLSDYNIL | CTTGCTTTTCAAGGGAAGAGCCTGGAGGACG |
| | | KDPKKMPLLRLR | GTCGCACACTGTCTGACTATAACATTCTTAAA |
| | | | GATCCTAAAAAGATGCCACTGCTGCGCTTGC |
| | | | GT |
| CM80 | K6R, T7M, | MLIFVRMLTGK | ATGTTGATTTTCGTACGCATGTTGACTGGAAA |
| [513; 730] | T12M, T14E, | MIELEVEPSDTIE | GATGATCGAGTTGGAAGTGGAGCCTTCCGAT |
| | K330, A46Q, | NVKAKIQDQEGI | ACTATCGAGAATGTTAAGGCCAAAATCCAAG |
| | S65P, L67K, | PPDQQRLAFQG | ATCAAGAAGGGATTCCTCCAGATCAACAACG |
| | H68M | KSLEDGRTLSDY | CCTTGCTTTTCAAGGGAAGAGCCTGGAGGAC |
| | | NILKDPKKMPLLR | GGTCGCACACTGTCTGACTATAACATTCTTAA |
| | | LR | AGATCCTAAAAAGATGCCACTGCTGCGCTTG |
| | | | CGT |
| CM81 | K6R, T7M, | MLIFVRMLTGK | ATGTTGATTTTCGTACGCATGTTGACTGGAAA |
| [514; 731] | T12M, T14E, | MIELEVEPSDTIE | GATGATCGAGTTGGAAGTGGAGCCTTCCGAT |
| | K33A, A46Q, | NVKAKIQDAEGI | ACTATCGAGAATGTTAAGGCCAAAATCCAAG |
| | S65P, L67K, | PPDQQRLAFQG | ATGCCGAAGGGATTCCTCCAGATCAACAACG |
| | H68M | KSLEDGRTLSDY | CCTTGCTTTTCAAGGGAAGAGCCTGGAGGAC |
| | | NILKDPKKMPLLR | GGTCGCACACTGTCTGACTATAACATTCTTAA |
| | | LR | AGATCCTAAAAAGATGCCACTGCTGCGCTTG |
| | | | CGT |
| CM82 | K6R, T7M, | MLIFVRMLTGK | ATGTTGATTTTCGTACGCATGTTGACTGGAAA |
| [515; 732] | T12M, T14E, | MIELEVEPSDTIE | GATGATCGAGTTGGAAGTGGAGCCTTCCGAT |
| | K33H, A46Q, | NVKAKIQDHEGI | ACTATCGAGAATGTTAAGGCCAAAATCCAAG |
| | S65P, L67K, | PLDQQRLAFQGK | ATCATGAAGGGATTCCTTTGGATCAACAACG |
| | H68M, P38L | SLEDGRTLSDYNI | CCTTGCTTTTCAAGGGAAGAGCCTGGAGGAC |
| | | LKDPKKMPLLRL | GGTCGCACACTGTCTGACTATAACATTCTTAA |
| | | R | AGATCCTAAAAAGATGCCACTGCTGCGCTTG |
| | | | CGT |
| CM83 | K6R, T7M, | MLIFVRMLTGK | ATGTTGATTTTCGTACGCATGTTGACTGGAAA |
| [516; 733] | T12M, T14E, | MIELEVEPSDTIE | GATGATCGAGTTGGAAGTGGAGCCTTCCGAT |
| | K33H, A46Q, | NVKAKIQDHEGI | ACTATCGAGAATGTTAAGGCCAAAATCCAAG |
| | S65P, L67K, | PCDQQRLAFQG | ATCATGAAGGGATTCCTTGCGATCAACAACG |
| | H68M, P38C | KSLEDGRTLSDY | CCTTGCTTTTCAAGGGAAGAGCCTGGAGGAC |
| | | NILKDPKKMPLLR | GGTCGCACACTGTCTGACTATAACATTCTTAA |
| | | LR | AGATCCTAAAAAGATGCCACTGCTGCGCTTG |
| | | | CGT |
| CM84 | K6R, T7M, | MLIFVRMLTGK | ATGTTGATTTTCGTACGCATGTTGACTGGAAA |
| [517; 734] | T12M, T14E, | MIELEVEPSDTIE | GATGATCGAGTTGGAAGTGGAGCCTTCCGAT |
| | K33H, A46Q, | NVKAKIQDHEGI | ACTATCGAGAATGTTAAGGCCAAAATCCAAG |
| | S65P, L67K, | PPDEQRLAFQGK | ATCATGAAGGGATTCCTCCAGATGAGCAACG |
| | H68M, Q40E | SLEDGRTLSDYNI | CCTTGCTTTTCAAGGGAAGAGCCTGGAGGAC |
| | | LKDPKKMPLLRL | GGTCGCACACTGTCTGACTATAACATTCTTAA |
| | | R | AGATCCTAAAAAGATGCCACTGCTGCGCTTG |
| | | | CGT |
| CM87 | K6R, T7M, | MLIFVRMLTGK | ATGTTGATTTTCGTACGCATGTTGACTGGAAA |
| [518; 735] | T12M, T14E, | MIELEVEPSDTIE | GATGATCGAGTTGGAAGTGGAGCCTTCCGAT |
| | K33H, A46Q, | NVKAKIQDHEGI | ACTATCGAGAATGTTAAGGCCAAAATCCAAG |
| | S65P, L67K, | PPDQQHLAFQG | ATCATGAAGGGATTCCTCCAGATCAACAACAT |
| | H68M, R42H | KSLEDGRTLSDY | CTTGCTTTTCAAGGGAAGAGCCTGGAGGACG |
| | | NILKDPKKMPLLR | GTCGCACACTGTCTGACTATAACATTCTTAAA |
| | | LR | GATCCTAAAAAGATGCCACTGCTGCGCTTGC |
| | | | GT |
| CM88 | K6R, T7M, | MLIFVRMLTGK | ATGTTGATTTTCGTACGCATGTTGACTGGAAA |
| [519; 736] | T12M, T14E, | MIELEVEPSDTIE | GATGATCGAGTTGGAAGTGGAGCCTTCCGAT |
| | K33H, A46Q, | NVKAKIQDHEGI | ACTATCGAGAATGTTAAGGCCAAAATCCAAG |
| | S65P, L67K, | PPDQQFLAFQGK | ATCATGAAGGGATTCCTCCAGATCAACAATTC |
| | H68M, R42F | SLEDGRTLSDYNI | CTTGCTTTTCAAGGGAAGAGCCTGGAGGACG |
| | | LKDPKKMPLLRL | GTCGCACACTGTCTGACTATAACATTCTTAAA |
| | | R | GATCCTAAAAAGATGCCACTGCTGCGCTTGC |
| | | | GT |
| CM89 | K6R, T7M, | MLIFVRMLTGK | ATGTTGATTTTCGTACGCATGTTGACTGGAAA |
| [520; 737] | T12M, T14E, | MIELEVEPSDTIE | GATGATCGAGTTGGAAGTGGAGCCTTCCGAT |
| | K33H, A46Q, | NVKAKIQDHEGI | ACTATCGAGAATGTTAAGGCCAAAATCCAAG |
| | S65P, L67K, | PPDQQRLTFQGK | ATCATGAAGGGATTCCTCCAGATCAACAACG |
| | H68M, A44T | SLEDGRTLSDYNI | CCTTACTTTTCAAGGGAAGAGCCTGGAGGAC |
| | | LKDPKKMPLLRL | GGTCGCACACTGTCTGACTATAACATTCTTAA |
| | | R | AGATCCTAAAAAGATGCCACTGCTGCGCTTG |
| | | | CGT |
| CM90 | K6R, T7M, | MLIFVRMLTGK | ATGTTGATTTTCGTACGCATGTTGACTGGAAA |
| [521; 738] | T12M, T14E, | MIELEVEPSDTIE | GATGATCGAGTTGGAAGTGGAGCCTTCCGAT |
| | K33H, A46Q, | NVKAKIQDHEGI | ACTATCGAGAATGTTAAGGCCAAAATCCAAG |
| | S65P, L67K, | PPDQQRLAFQGT | ATCATGAAGGGATTCCTCCAGATCAACAACG |
| | H68M, K48T | SLEDGRTLSDYNI | CCTTGCTTTTCAAGGGACTAGCCTGGAGGAC |
| | | LKDPKKMPLLRL | GGTCGCACACTGTCTGACTATAACATTCTTAA |
| | | R | AGATCCTAAAAAGATGCCACTGCTGCGCTTG |
| | | | CGT |
| CM92 | K6R, T7M, | MLIFVRMLTGK | ATGTTGATTTTCGTACGCATGTTGACTGGAAA |
| [522; 739] | T12M,T14E, | MIELEVEPSDTIE | GATGATCGAGTTGGAAGTGGAGCCTTCCGAT |
| | K33H, A46Q, | NVKAKIQDHEGI | ACTATCGAGAATGTTAAGGCCAAAATCCAAG |
| | S65P, L67K, | PPDQQRLAFQG | ATCATGAAGGGATTCCTCCAGATCAACAACG |
| | H68M, S49L | KLLEDGRTLSDYN | CCTTGCTTTTCAAGGGAAGTTGCTGGAGGAC |
| | | ILKDPKKMPLLRL | GGTCGCACACTGTCTGACTATAACATTCTTAA |
| | | R | AGATCCTAAAAAGATGCCACTGCTGCGCTTG |
| | | | CGT |
| CM93 | K6R, T7M, | MLIFVRMLTGK | ATGTTGATTTTCGTACGCATGTTGACTGGAAA |
| [523; 740] | T12M, T14E, | MIELEVEPSDTIE | GATGATCGAGTTGGAAGTGGAGCCTTCCGAT |
| | K33H, A46Q, | NVKAKIQDHEGI | ACTATCGAGAATGTTAAGGCCAAAATCCAAG |
| | S65P, L67K, | PPDQQRLAFQG | ATCATGAAGGGATTCCTCCAGATCAACAACG |
| | H68M, S49M | KMLEDGRTLSDY | CCTTGCTTTTCAAGGGAAGATGCTGGAGGAC |
| | | NILKDPKKMPLLR | GGTCGCACACTGTCTGACTATAACATTCTTAA |
| | | LR | AGATCCTAAAAAGATGCCACTGCTGCGCTTG |
| | | | CGT |
| CM94 | K6R, T7M, | MLIFVRMLTGK | ATGTTGATTTTCGTACGCATGTTGACTGGAAA |
| [524; 741] | T12M, T14E, | MIELEVEPSDTIE | GATGATCGAGTTGGAAGTGGAGCCTTCCGAT |
| | K33H, A46Q, | NVKAKIQDHEGI | ACTATCGAGAATGTTAAGGCCAAAATCCAAG |
| | S65P, L67K, | PPDQQRLAFQG | ATCATGAAGGGATTCCTCCAGATCAACAACG |
| | H68M, E51D | KSLDDGRTLSDY | CCTTGCTTTTCAAGGGAAGAGCCTGGATGAC |
| | | NILKDPKKMPLLR | GGTCGCACACTGTCTGACTATAACATTCTTAA |
| | | LR | AGATCCTAAAAAGATGCCACTGCTGCGCTTG |
| | | | CGT |
| CM95 | K6R, T7M, | MLIFVRMLTGK | ATGTTGATTTTCGTACGCATGTTGACTGGAAA |
| [525; 742] | T12M, T14E, | MIELEVEPSDTIE | GATGATCGAGTTGGAAGTGGAGCCTTCCGAT |
| | K33H, A46Q, | NVKAKIQDHEGI | ACTATCGAGAATGTTAAGGCCAAAATCCAAG |
| | S65P, L67K, | PPDQQRLAFQG | ATCATGAAGGGATTCCTCCAGATCAACAACG |
| | H68M, R54Y | KSLEDGYTLSDYN | CCTTGCTTTTCAAGGGAAGAGCCTGGAGGAC |
| | | ILKDPKKMPLLRL | GGTTATACACTGTCTGACTATAACATTClTAA |
| | | R | AGATCCTAAAAAGATGCCACTGCTGCGCTTG |
| | | | CGT |
| CM98 | K6R, T7M, | MLIFVRMLTGK | ATGTTGATTTTCGTACGCATGTTGACTGGAAA |
| [526; 743] | T12M, T14E, | MIELEVEPSDTIE | GATGATCGAGTTGGAAGTGGAGCCTTCCGAT |
| | K33H, A46Q, | NVKAKIQDHEGI | ACTATCGAGAATGTTAAGGCCAAAATCCAAG |
| | S65P, L67K, | PPDQQRLAFQG | ATCATGAAGGGATTCCTCCAGATCAACAACG |
| | H68M, S57G | KSLEDGRTLGDY | CCTTGCTTTTCAAGGGAAGAGCCTGGAGGAC |
| | | NILKDPKKMPLLR | GGTCGCACACTGGGGGACTATAACATTCTTA |
| | | LR | AAGATCCTAAAAAGATGCCACTGCTGCGCTT |
| | | | GCGT |
| CM101 | K6R, T7M, | MLIFVRMLTGK | ATGTTGATTTTCGTACGCATGTTGACTGGAAA |
| [527; 744] | T12M, T14E, | MIELEVEPSDTIE | GATGATCGAGTTGGAAGTGGAGCCTTCCGAT |
| | K33H, A46Q, | NVKAKIQDHEGI | ACTATCGAGAATGTTAAGGCCAAAATCCAAG |
| | S65P, L67K, | PPDQQRLAFQG | ATCATGAAGGGATTCCTCCAGATCAACAACG |
| | H68M, I61L | KSLEDGRTLSDY | CCTTGCTTTTCAAGGGAAGAGCCTGGAGGAC |
| | | NLLKDPKKMPLL | GGTCGCACACTGTCTGACTATAACTTGCTTAA |
| | | RLR | AGATCCTAAAAAGATGCCACTGCTGCGCTTG |
| | | | CGT |
| CM102 | K6R, T7M, | MLIFVRMLTGK | ATGTTGATTTTCGTACGCATGTTGACTGGAAA |
| [528; 745] | T12M, T14E, | MIELEVEPSDTIE | GATGATCGAGTTGGAAGTGGAGCCTTCCGAT |
| | K33H, A46Q, | NVKAKIQDHEGI | ACTATCGAGAATGTTAAGGCCAAAATCCAAG |
| | S65P, L67K, | PPDQQRLAFQG | ATCATGAAGGGATTCCTCCAGATCAACAACG |
| | H68M, K63I | KSLEDGRTLSDY | CCTTGCTTTTCAAGGGAAGAGCCTGGAGGAC |
| | | NILIDPKKMPLLR | GGTCGCACACTGTCTGACTATAACATTCTTAT |
| | | LR | TGATCCTAAAAAGATGCCACTGCTGCGCTTGC |
| | | | GT |
| CM103 | K6R, T7M, | MLIFVRMLTGK | ATGTTGATTTTCGTACGCATGTTGACTGGAAA |
| [529; 746] | T12M, T14E, | MIELEVEPSDTIE | GATGATCGAGTTGGAAGTGGAGCCTTCCGAT |
| | K33H, A46Q, | NVKAKIQDHEGI | ACTATCGAGAATGTTAAGGCCAAAATCCAAG |
| | S65H, L67K, | PPDQQRLAFQG | ATCATGAAGGGATTCCTCCAGATCAACAACG |
| | H68M | KSLEDGRTLSDY | CCTTGCTTTTCAAGGGAAGAGCCTGGAGGAC |
| | | NILKDHKKMPLL | GGTCGCACACTGTCTGACTATAACATTCTTAA |
| | | RLR | AGATCATAAAAAGATGCCACTGCTGCGCTTG |
| | | | CGT |
| CM104 | K6R, T7M, | MLIFVRMLTGK | ATGTTGATTTTCGTACGCATGTTGACTGGAAA |
| [530; 747] | T12M, T14E, | MIELEVEPSDTIE | GATGATCGAGTTGGAAGTGGAGCCTTCCGAT |
| | K33H, A46Q, | NVKAKIQDHEGI | ACTATCGAGAATGTTAAGGCCAAAATCCAAG |
| | S65P, L67K, | PPDQQRLAFQG | ATCATGAAGGGATTCCTCCAGATCAACAACG |
| | H68M, L73M | KSLEDGRTLSDY | CCTTGCTTTTCAAGGGAAGAGCCTGGAGGAC |
| | | NILKDPKKMPLLR | GGTCGCACACTGTCTGACTATAACATTCTTAA |
| | | MR | AGATCCTAAAAAGATGCCACTGCTGCGCATG |
| | | | CGT |
| CM105 | K6R, T7M, | MLIFVRMLTGK | ATGTTGATTTTCGTACGCATGTTGACTGGAAA |
| [531; 748] | T12M, T14E, | MIELEVEPSDTIE | GATGATCGAGTTGGAAGTGGAGCCTTCCGAT |
| | K33H, A46Q, | NVKAKIQDHEGI | ACTATCGAGAATGTTAAGGCCAAAATCCAAG |
| | S65P, L67K, | PPDQQRLAFQG | ATCATGAAGGGATTCCTCCAGATCAACAACG |
| | H68M, R74Q | KSLEDGRTLSDY | CCTTGCTTTTCAAGGGAAGAGCCTGGAGGAC |
| | | NILKDPKKMPLLR | GGTCGCACACTGTCTGACTATAACATTCTTAA |
| | | LQ | AGATCCTAAAAAGATGCCACTGCTGCGCTTG |
| | | | CAA |
| CM107 | T7M, T12M, | MLIFVKMLTGK | ATGTTGATTTTCGTACGCATGTTGACTGGAAA |
| [532; 749] | T14E, K33H, | MIELEVEPSDTIE | GATGATCGAGTTGGAAGTGGAGCCTTCCGAT |
| | A46Q, S65P, | NVKAKIQDHEGI | ACTATCGAGAATGTTAAGGCCAAAATCCAAG |
| | L67K, H68M, | PPDQQRLAFQG | ATCATGAAGGGATTCCTCCAGATCAACAACG |
| | P69L, L70V | KSLEDGRTLSDY | CCTTGCTTTTCAAGGGAAGAGCCTGGAGGAC |
| | | NILKDPKKMLVL | GGTCGCACACTGTCTGACTATAACATTCTTAA |
| | | RLR | AGATCCTAAAAAGATGTTGGTACTGCGCTTG |
| | | | CGT |
| CM108 | K6R, T7M, | MLIFVRMLTGK | ATGTTGATTTTCGTACGCATGTTGACTGGAAA |
| [533; 750] | T12M, T14E, | MIELEVYPSDTIE | GATGATCGAGTTGGAAGTGTATCCTTCCGAT |
| | K33H, A46Q, | NVKAKIQDHEGI | ACTATCGAGAATGTTAAGGCCAAAATCCAAG |
| | S65P, L67K, | PLDQQRLAFQGK | ATCATGAAGGGATTCCTTTGGATCAACAACG |
| | H68M, E18Y, | LLEDGRTLGDYNI | CCTTGCTTTTCAAGGGAAGTTGCTGGAGGAC |
| | P38L, S49L, | LKDPKKMPLLRL | GGTCGCACACTGGGGGACTATAACATTCTTA |
| | S57G | R | AAGATCCTAAAAAGATGCCACTGCTGCGCTT |
| | | | GCGT |
| CM110 | K6R, T7M, | HLIFVRMLTGKM | CATTTGATTTTCGTACGCATGTTGACTGGAAA |
| [534; 751] | T12M, T14E, | IELEVEPSDTIENV | GATGATCGAGTTGGAAGTGGAGCCTTCCGAT |
| | K33H, A46Q, | KAKIQDHEGIPP | ACTATCGAGAATGTTAAGGCCAAAATCCAAG |
| | S65P, L67K, | DQQRLAFQGKSL | ATCATGAAGGGATTCCTCCAGATCAACAACG |
| | H68M, M1H, | EDGRTLSDYNILK | CCTTGCTTTTCAAGGGAAGAGCCTGGAGGAC |
| | R74Q | DPKKMPLLRLQ | GGTCGCACACTGTCTGACTATAACATTCTTAA |
| | | | AGATCCTAAAAAGATGCCACTGCTGCGCTTG |
| | | | CAA |
| CM111 | K6R, T7M, | YLIFVRMLTGKMI | TATTTGATTTTCGTACGCATGTTGACTGGAAA |
| [535; 752] | T12M, T14E, | ELEVEPSDTIENIK | GATGATCGAGTTGGAAGTGGAGCCTTCCGAT |
| | K33H, A46Q, | AKIQDHEGIPPD | ACTATCGAGAATATTAAGGCCAAAATCCAAG |
| | S65P, L67K, | QQRLAFQGKSLE | ATCATGAAGGGATTCCTCCAGATCAACAACG |
| | H68M, M1Y, | DGRTLSDYNILKD | CCTTGCTTTTCAAGGGAAGAGCCTGGAGGAC |
| | V26I, L73M | PKKMPLLRMR | GGTCGCACACTGTCTGACTATAACATTCTTAA |
| | | | AGATCCTAAAAAGATGCCACTGCTGCGCATG |
| | | | CGT |
| CM112 | K6R, T7M, | MLIFVRMLTGK | ATGTTGATTTTCGTACGCATGTTGACTGGAAA |
| [536; 753] | T12M, T14E, | MIELEVEPSDTIE | GATGATCGAGTTGGAAGTGGAGCCTTCCGAT |
| | K33H, A46Q, | VVKAKIQDHEGI | ACTATCGAGGTAGTTAAGGCCAAAATCCAAG |
| | S65P, L67K, | PPDEQRLAFQGK | ATCATGAAGGGATTCCTCCAGATGAGCAACG |
| | H68M, N25V, | SLDDGRTLSDYNI | CCTTGCTTTTCAAGGGAAGAGCCTGGATGAC |
| | Q40E, E51D | LKDPKKMPLLRL | GGTCGCACACTGTCTGACTATAACATTCTTAA |
| | | R | AGATCCTAAAAAGATGCCACTGCTGCGCTTG |
| | | | CGT |
| CM113 | K6R, T7M, | MLIFVRMLTGK | ATGTTGATTTTCGTACGCATGTTGACTGGAAA |
| [537; 754] | T12M, T14E, | MIELEVEPSDTIE | GATGATCGAGTTGGAAGTGGAGCCTTCCGAT |
| | K33H, A46Q, | NVKAKIQDHEGI | ACTATCGAGAATGTTAAGGCCAAAATCCAAG |
| | S65P, L67K, | PPDQQRLAFQG | ATCATGAAGGGATTCCTCCAGATCAACAACG |
| | H68M, I61L, | KSLEDGRTLSDY | CCTTGCTTTTCAAGGGAAGAGCCTGGAGGAC |
| | K63I | NLLIDPKKMPLLR | GGTCGCACACTGTCTGACTATAACTTGCTTAT |
| | | LR | TGATCCTAAAAAGATGCCACTGCTGCGCTTGC |
| | | | GT |
| CM114 | K6R, T7M, | MLIFVRMLTGK | ATGTTGATTTTCGTACGCATGTTGACTGGAAA |
| [538; 755] | T12M, T14E, | MIELEVMPSDTIE | GATGATCGAGTTGGAAGTGATGCCTTCCGAT |
| | K33H, A46Q, | NVKAKIQDHEGI | ACTATCGAGAATGTTAAGGCCAAAATCCAAG |
| | S65P, L67K, | PPDQQRLAFQGT | ATCATGAAGGGATTCCTCCAGATCAACAACG |
| | H68M, E18M, | SLDDGRTLGDYN | CCTTGCTTTTCAAGGGACTAGCCTGGATGACG |
| | K48T, E51D, | ILKDPKKMPLLRL | GTCGCACACTGGGGGACTATAACATTCTTAA |
| | S57G | R | AGATCCTAAAAAGATGCCACTGCTGCGCTTG |
| | | | CGT |
| CM115 | K6R, T7M, | MLIFVRMLTGK | ATGTTGATTTTCGTACGCATGTTGACTGGAAA |
| [539; 756] | T12M, T14E, | MIELMVEPSDTIE | GATGATCGAGTTGATGGTGGAGCCTTCCGAT |
| | K33H, A46Q, | VVKAKIQDHEGI | ACTATCGAGGTAGTTAAGGCCAAAATCCAAG |
| | S65P, L67K, | PPDEQRLAFQGK | ATCATGAAGGGATTCCTCCAGATGAGCAACG |
| | H68M, E16M, | LLEDGRTLSDYNI | CCTTGCTTTTCAAGGGAAGTTGCTGGAGGAC |
| | N25V, Q40E, | LKDPKKMPLLRL | GGTCGCACACTGTCTGACTATAACATTCTTAA |
| | S49L | R | AGATCCTAAAAAGATGCCACTGCTGCGCTTG |
| | | | CGT |
| CM116 | K6R, T7M, | MLIFVRMLTGK | ATGTTGATTTTCGTACGCATGTTGACTGGAAA |
| [540; 757] | T12M, T14E, | MIELEVEPSDTIE | GATGATCGAGTTGGAAGTGGAGCCTTCCGAT |
| | K33H, A46Q, | NIKAKIQDHEGIP | ACTATCGAGAATATTAAGGCCAAAATCCAAG |
| | S65P, L67K, | PDQYRLAFQGKL | ATCATGAAGGGATTCCTCCAGATCAATATCGC |
| | H68M, V26I, | LEDGRTLGDYNIL | CTTGCTTTTCAAGGGAAGTTGCTGGAGGACG |
| | Q41Y, S49L, | KDPKKMPLLRLR | GTCGCACACTGGGGGACTATAACATTCTTAA |
| | S57G | | AGATCCTAAAAAGATGCCACTGCTGCGCTTG |
| | | | CGT |
| CM117 | K6R, T7M, | MLIFVRMLTGK | ATGTTGATTTTCGTACGCATGTTGACTGGAAA |
| [541; 758] | T12M, T14E, | MIELEVEPSDTIE | GATGATCGAGTTGGAAGTGGAGCCTTCCGAT |
| | K33H, A46Q, | NVKAKICDHEGIP | ACTATCGAGAATGTTAAGGCCAAAATCTGCG |
| | S65P, L67K, | PDQQHLAFQGK | ATCATGAAGGGATTCCTCCAGATCAACAACAT |
| | H68M, Q31C, | SLEDGRTLGDYNI | CTTGCTTTTCAAGGGAAGAGCCTGGAGGACG |
| | R42H,S57G | LKDPKKMPLLRL | GTCGCACACTGGGGGACTATAACATTCTTAA |
| | | R | AGATCCTAAAAAGATGCCACTGCTGCGCTTG |
| | | | CGT |
| CM118 | K6R, T7M, | MLIFVRMLTGK | ATGTTGATTTTCGTACGCATGTTGACTGGAAA |
| [542; 759] | T12M, T14E, | MIELEVMPSDTIE | GATGATCGAGTTGGAAGTGATGCCTTCCGAT |
| | K33H, A46Q, | WKAKIFDHEGIP | ACTATCGAGGTAGTTAAGGCCAAAATCTTCG |
| | S65P, L67K, | PDQQHLAFQGT | ATCATGAAGGGATTCCTCCAGATCAACAACAT |
| | H68M, E18M, | SLEDGYTLGDYNI | CTTGCTTTTCAAGGGACTAGCCTGGAGGACG |
| | N25V, Q31F, | LKDPKKMPLLRL | GTTATACACTGGGGGACTATAACATTCTTAAA |
| | R42H, K48T, | R | GATCCTAAAAAGATGCCACTGCTGCGCTTGC |
| | R54Y, S57G | | GT |
| CM119 | K6R, T7M, | MLIFVRMLTGK | ATGTTGATTTTCGTACGCATGTTGACTGGAAA |
| [543; 760] | T12M, T14E, | MIELEVMPSDTIE | GATGATCGAGTTGGAAGTGATGCCTTCCGAT |
| | K33H, A46Q, | WKAKIFDHEGIP | ACTATCGAGGTAGTTAAGGCCAAAATCTTCG |
| | S65P, L67K, | PDQQHLTFQGTL | ATCATGAAGGGATTCCTCCAGATCAACAACAT |
| | H68M, E18M, | LEDGYTLGDYNIL | CTTACTTTTCAAGGGACTTTGCTGGAGGACG |
| | N25V, Q31F, | KDPKKMPLLRLR | GTTATACACTGGGGGACTATAACATTCTTAAA |
| | R42H, A44T, | | GATCCTAAAAAGATGCCACTGCTGCGCTTGC |
| | K48T, S49L, | | GT |
| | R54Y, S57G | | |
| CM120 | K6R, T7M, | MLIFVRMLTGK | ATGTTGATTTTCGTACGCATGTTGACTGGAAA |
| [544; 761] | T12M, T14E, | MIELEVMPSDTIE | GATGATCGAGTTGGAAGTGATGCCTTCCGAT |
| | K33H, A46Q, | WKAKIFDHEGIP | ACTATCGAGGTAGTTAAGGCCAAAATCTTCG |
| | S65P, L67K, | LDQQHLAFQGTS | ATCATGAAGGGATTCCTTTGGATCAACAACAT |
| | H68M, E18M, | LEDGYTLGDYNIL | CTTGCTTTTCAAGGGACTAGCCTGGAGGACG |
| | N25V, Q31F, | KDPKKMPLLRLR | GTTATACACTGGGGGACTATAACATTCTTAAA |
| | P38L, R42H, | | GATCCTAAAAAGATGCCACTGCTGCGCTTGC |
| | K48T, R54Y, | | GT |
| | S57G | | |
| CM121 | K6R, T7M, | MLIFVRMLTGK | ATGTTGATTTTCGTACGCATGTTGACTGGAAA |
| [545; 762] | T12M, T14E, | MIELEVMPSDTIE | GATGATCGAGTTGGAAGTGATGCCTTCCGAT |
| | K33H, A46Q, | VVKAKIQDHEGI | ACTATCGAGGTAGTTAAGGCCAAAATCCAAG |
| | S65P, L67K, | PPDQQHLAFQG | ATCATGAAGGGATTCCTCCAGATCAACAACAT |
| | H68M, E18M, | TSLEDGYTLGDY | CTTGCTTTTCAAGGGACTAGCCTGGAGGACG |
| | N25V, R42H, | NILKDPKKMPLLR | GTTATACACTGGGGGACTATAACATTCTTAAA |
| | K48T, R54Y, | LR | GATCCTAAAAAGATGCCACTGCTGCGCTTGC |
| | S57G | | GT |
| CM131 | K6R, T7M, | YLIFVRMLTGKMI | TATTTGATTTTCGTACGCATGTTGACTGGAAA |
| [546; 763] | T12M, T14E, | ELEVEPSDTIENIK | GATGATCGAGTTGGAAGTGGAGCCTTCCGAT |
| | K33H, A46Q, | AKIQDHEGIPPD | ACTATCGAGAATATTAAGGCCAAAATCCAAG |
| | S65P, L67K, | QQRLAFQGKSLE | ATCATGAAGGGATTCCTCCAGATCAACAACG |
| | H68M, I61L, | DGRTLSDYNLLID | CCTTGCTTTTCAAGGGAAGAGCCTGGAGGAC |
| | K63I, M1Y, | PKKMPLLRMR | GGTCGCACACTGTCTGACTATAACTTGCTTAT |
| | V26I, L73M | | TGATCCTAAAAAGATGCCACTGCTGCGCATG |
| | | | CGT |
| CM132 | K6R, T7M, | YLIFVRMLTGKMI | TATTTGATTTTCGTACGCATGTTGACTGGAAA |
| [547; 764] | T12M, T14E, | ELEVEPSDTIEVIK | GATGATCGAGTTGGAAGTGGAGCCTTCCGAT |
| | K33H, A46Q, | AKIQDHEGIPPDE | ACTATCGAGGTAATTAAGGCCAAAATCCAAG |
| | S65P, L67K, | QRLAFQGKSLDD | ATCATGAAGGGATTCCTCCAGATGAGCAACG |
| | H68M, I61L, | GRTLSDYNLLIDP | CCTTGCTTTTCAAGGGAAGAGCCTGGATGAC |
| | K63I, M1Y, | KKMPLLRMR | GGTCGCACACTGTCTGACTATAACTTGCTTAT |
| | V26I, L73M, | | TGATCCTAAAAAGATGCCACTGCTGCGCATG |
| | N25V, Q40E, | | CGT |
| | E51D | | |
| CM133 | K6R, T7M, | MLIFVRMLTGK | ATGTTGATTTTCGTACGCATGTTGACTGGAAA |
| [548; 765] | T12M, T14E, | MIELEVEPSDTIE | GATGATCGAGTTGGAAGTGGAGCCTTCCGAT |
| | K33H, A46Q, | VVKAKIQDHEGI | ACTATCGAGGTAGTTAAGGCCAAAATCCAAG |
| | S65P, L67K, | PPDEQRLAFQGK | ATCATGAAGGGATTCCTCCAGATGAGCAACG |
| | H68M, I61L, | SLDDGRTLSDYN | CCTTGCTTTTCAAGGGAAGAGCCTGGATGAC |
| | K63I, N25V, | LLIDPKKMPLLRL | GGTCGCACACTGTCTGACTATAACTTGCTTAT |
| | Q40E, E51D | R | TGATCCTAAAAAGATGCCACTGCTGCGCTTGC |
| | | | GT |
| CM134 | K6R, T7M, | MLIFVRMLTGK | ATGTTGATTTTCGTACGCATGTTGACTGGAAA |
| [549; 766] | T12M, T14E, | MIELEVMPSDTIE | GATGATCGAGTTGGAAGTGATGCCTTCCGAT |
| | K33H, A46Q, | NVKAKIQDHEGI | ACTATCGAGAATGTTAAGGCCAAAATCCAAG |
| | S65P, L67K, | PPDQQRLAFQGT | ATCATGAAGGGATTCCTCCAGATCAACAACG |
| | H68M, I61L, | SLDDGRTLGDYN | CCTTGCTTTTCAAGGGACTAGCCTGGATGACG |
| | K63I, E18M, | LLIDPKKMPLLRL | GTCGCACACTGGGGGACTATAACTTGCTTATT |
| | K48T, E51D, | R | GATCCTAAAAAGATGCCACTGCTGCGCTTGC |
| | S57G | | GT |
| CM135 | K6R, T7M, | MLIFVRMLTGK | ATGTTGATTTTCGTACGCATGTTGACTGGAAA |
| [550; 767] | T12M, T14E, | MIELMVEPSDTIE | GATGATCGAGTTGATGGTGGAGCCTTCCGAT |
| | K33H, A46Q, | VVKAKIQDHEGI | ACTATCGAGGTAGTTAAGGCCAAAATCCAAG |
| | S65P, L67K, | PPDEQRLAFQGK | ATCATGAAGGGATTCCTCCAGATGAGCAACG |
| | H68M, I61L, | LLEDGRTLSDYNL | CCTTGCTTTTCAAGGGAAGTTGCTGGAGGAC |
| | K63I, E16M, | LIDPKKMPLLRLR | GGTCGCACACTGTCTGACTATAACTTGCTTAT |
| | N25V, Q40E, | | TGATCCTAAAAAGATGCCACTGCTGCGCTTGC |
| | S49L | | GT |
| CM136 | K6R, T7M, | YLIFVRMLTGKMI | TATTTGATTTTCGTACGCATGTTGACTGGAAA |
| [551; 768] | T12M, T14E, | ELEVMPSDTIENI | GATGATCGAGTTGGAAGTGATGCCTTCCGAT |
| | K33H, A46Q, | KAKIQDHEGIPP | ACTATCGAGAATATTAAGGCCAAAATCCAAG |
| | S65P, L67K, | DQQRLAFQGTSL | ATCATGAAGGGATTCCTCCAGATCAACAACG |
| | H68M, I61L, | DDGRTLGDYNLLI | CCTTGCTTTTCAAGGGACTAGCCTGGATGACG |
| | K63I, E18M, | DPKKMPLLRMR | GTCGCACACTGGGGGACTATAACTTGCTTATT |
| | K48T, E51D, | | GATCCTAAAAAGATGCCACTGCTGCGCATGC |
| | S57G, M1Y, | | GT |
| | V26I, L73M | | |
| CM137 | K6R, T7M, | YLIFVRMLTGKMI | TATTTGATTTTCGTACGCATGTTGACTGGAAA |
| [552; 769] | T12M, T14E, | ELEVMPSDTIENI | GATGATCGAGTTGGAAGTGATGCCTTCCGAT |
| | K33H, A46Q, | KAKIQDHEGIPP | ACTATCGAGAATATTAAGGCCAAAATCCAAG |
| | S65P, L67K, | DQQRLAFQGTSL | ATCATGAAGGGATTCCTCCAGATCAACAACG |
| | H68M, I61L, | DDGRTLGDYNLLI | CCTTGCTTTTCAAGGGACTAGCCTGGATGACG |
| | K63I, E18M, | DPKKMPLLRMQ | GTCGCACACTGGGGGACTATAACTTGCTTATT |
| | K48T, E51D, | | GATCCTAAAAAGATGCCACTGCTGCGCATGC |
| | S57G, M1Y, | | AA |
| | V26I, L73M, | | |
| | R74Q | | |
| CM138 | K6R, T7M, | YLIFVRMLTGKMI | TATTTGATTTTCGTACGCATGTTGACTGGAAA |
| [553; 770] | T12M, T14E, | ELEVMPSDTIEVI | GATGATCGAGTTGGAAGTGATGCCTTCCGAT |
| | K33H, A46Q, | KAKIQDHEGIPP | ACTATCGAGGTAATTAAGGCCAAAATCCAAG |
| | S65P, L67K, | DEQRLAFQGTSL | ATCATGAAGGGATTCCTCCAGATGAGCAACG |
| | H68M, I61L, | DDGRTLGDYNLLI | CCTTGCTTTTCAAGGGACTAGCCTGGATGACG |
| | K63I, E18M, | DPKKMPLLRMQ | GTCGCACACTGGGGGACTATAACTTGCTTATT |
| | K48T, E51D, | | GATCCTAAAAAGATGCCACTGCTGCGCATGC |
| | S57G, M1Y, | | AA |
| | V26I, L73M, | | |
| | R74Q, N25V, | | |
| | Q40E, E51D | | |
| CM139 | K6R, T7M, | MLIFVRMLTGK | ATGTTGATTTTCGTACGCATGTTGACTGGAAA |
| [554; 771] | T12M, T14E, | MIELEVEPSDTIE | GATGATCGAGTTGGAAGTGGAGCCTTCCGAT |
| | K33H, A46Q, | NVKAKIQDHEGI | ACTATCGAGAATGTTAAGGCCAAAATCCAAG |
| | S65P, L67K, | PPDQQRLTFQGK | ATCATGAAGGGATTCCTCCAGATCAACAACG |
| | H68M, I61L, | LLEDGRTLSDYNL | CCTTACTTTTCAAGGGAAGTTGCTGGAGGAC |
| | K63I, A44T, | LIDPKKMPLLRLR | GGTCGCACACTGTCTGACTATAACTTGCTTAT |
| | S49L | | TGATCCTAAAAAGATGCCACTGCTGCGCTTGC |
| | | | GT |
| CM 140 | K6R, T7M, | MLIFVRMLTGK | ATGTTGATTTTCGTACGCATGTTGACTGGAAA |
| [555; 772] | T12M, T14E, | MIELEVEPSDTIE | GATGATCGAGTTGGAAGTGGAGCCTTCCGAT |
| | K33H, A46Q, | NVKAKIQDHEGI | ACTATCGAGAATGTTAAGGCCAAAATCCAAG |
| | S65P, L67K, | PLDQQRLTFQGK | ATCATGAAGGGATTCCTTTGGATCAACAACG |
| | H68M, I61L, | LLEDGRTLSDYNL | CCTTACTTTTLAAGGGAAGTTGCTGGAGGAC |
| | K63I, A44T, | LIDPKKMPLLRLR | GGTCGCACACTGTCTGACTATAACTTGCTTAT |
| | S49L, P38L | | TGATCCTAAAAAGATGCCACTGCTGCGCTTGC |
| | | | GT |
| CM141 | K6R, T7M, | MLIFVRMLTGK | ATGTTGATTTTCGTACGCATGTTGACTGGAAA |
| [556; 773] | T12M, T14E, | MIELEVEPSDTIE | GATGATCGAGTTGGAAGTGGAGCCTTCCGAT |
| | K33H, A46Q, | NVKAKIQDHEGI | ACTATCGAGAATGTTAAGGCCAAAATCCAAG |
| | S65P, L67K, | PLDQQRLTFQGK | ATCATGAAGGGATTCCTTTGGATCAACAACG |
| | H68M, I61L, | LLEDGRTLSDYNL | CCTTACTTTTCAAGGGAAGTTGCTGGAGGAC |
| | K63I, A44T, | LIDPKKMPLLRLQ | GGTCGCACACTGTCTGACTATAACTTGCTTAT |
| | S49L, P38L, | | TGATCCTAAAAAGATGCCACTGCTGCGCTTGC |
| | R74Q | | AA |
| CM142 | K6R, T7M, | YLIFVRMLTGKMI | TATTTGATTTTCGTACGCATGTTGACTGGAAA |
| [557; 774] | T12M, T14E, | ELEVEPSDTIENIK | GATGATCGAGTTGGAAGTGGAGCCTTCCGAT |
| | K33H, A46Q, | AKIQDHEGIPLD | ACTATCGAGAATATTAAGGCCAAAATCCAAG |
| | S65P, L67K, | QQRLTFQGKLLE | ATCATGAAGGGATTCCTTTGGATCAACAACG |
| | H68M, I61L, | DGRTLSDYNLLID | CCTTACTTTTLAAGGGAAGTTGCTGGAGGAC |
| | K63I, A44T, | PKKMPLLRMQ | GGTCGCACACTGTCTGACTATAACTTGCTTAT |
| | S49L, P38L, | | TGATCCTAAAAAGATGCCACTGCTGCGCATG |
| | R74Q, M1Y, | | CAA |
| | V26I, L73M | | |
| CM143 | K6R, T7M, | MLIFVRMLTGK | ATGTTGATTTTCGTACGCATGTTGACTGGAAA |
| [558; 775] | T12M, T14E, | MIELEVEPSDTIE | GATGATCGAGTTGGAAGTGGAGCCTTCCGAT |
| | K33H, A46Q, | VVKAKIQDHEGI | ACTATCGAGGTAGTTAAGGCCAAAATCCAAG |
| | S65P, L67K, | PLDEQRLTFQGK | ATCATGAAGGGATTCCTTTGGATGAGCAACG |
| | H68M, I61L, | LLDDGRTLSDYNL | CCTTACTTTTCAAGGGAAGTTGCTGGATGACG |
| | K63I, A44T, | LIDPKKMPLLRLQ | GTCGCACACTGTCTGACTATAACTTGCTTATT |
| | S49L, P38L, | | GATCCTAAAAAGATGCCACTGCTGCGCTTGC |
| | R74Q, N25V, | | AA |
| | Q40E, E51D | | |
| CM144 | K6R, T7M, | YLIFVRMLTGKMI | TATTTGATTTTCGTACGCATGTTGACTGGAAA |
| [559; 776] | T12M, T14E, | ELEVEPSDTIEVIK | GATGATCGAGTTGGAAGTGGAGCCTTCCGAT |
| | K33H, A46Q, | AKIQDHEGIPLDE | ACTATCGAGGTAATTAAGGCCAAAATCCAAG |
| | S65P, L67K, | QRLTFQGKLLDD | ATCATGAAGGGATTCCTTTGGATGAGCAACG |
| | H68M, I61L, | GRTLSDYNLLIDP | CCTTACTTTTCAAGGGAAGTTGCTGGATGACG |
| | K63I, A44T, | KKMPLLRMQ | GTCGCACACTGTCTGACTATAACTTGCTTATT |
| | S49L, P38L, | | GATCCTAAAAAGATGCCACTGCTGCGCATGC |
| | R74Q, N25V, | | AA |
| | Q40E, E51D, | | |
| | M1Y, V26I, | | |
| | L73M | | |
| CM145 | K6R, T7M, | MLIFVRMLTGK | ATGTTGATTTTCGTACGCATGTTGACTGGAAA |
| [560; 777] | T12M, T14E, | MIELEVMPSDTIE | GATGATCGAGTTGGAAGTGATGCCTTCCGAT |
| | K33H, A46Q, | VVKAKIQDHEGI | ACTATCGAGGTAGTTAAGGCCAAAATCCAAG |
| | S65P, L67K, | PPDQQHLTFQGT | ATCATGAAGGGATTCCTCCAGATCAACAACAT |
| | H68M, I61L, | LLEDGYTLGDYNL | CTTACTTTTCAAGGGACTTTGCTGGAGGACG |
| | K63I, E18M, | LIDPKKMPLLRLR | GTTATACACTGGGGGACTATAACTTGCTTATT |
| | N25V, R42H, | | GATCCTAAAAAGATGCCACTGCTGCGCTTGC |
| | K48T, R54Y, | | GT |
| | S57G, A44T, | | |
| | S49L | | |
| CM146 | K6R, T7M, | MLIFVRMLTGK | ATGTTGATTTTCGTACGCATGTTGACTGGAAA |
| [561; 778] | T12M, T14E, | MIELEVMPSDTIE | GATGATCGAGTTGGAAGTGATGCCTTCCGAT |
| | K33H, A46Q, | VVKAKIQDHEGI | ACTATCGAGGTAGTTAAGGCCAAAATCCAAG |
| | S65P, L67K, | PLDQQHLTFQGT | ATCATGAAGGGATTCCTTTGGATCAACAACAT |
| | H68M, I61L, | LLEDGYTLGDYNL | CTTACTTTTCAAGGGACTTTGCTGGAGGACG |
| | K63I, E18M, | LIDPKKMPLLRLR | GTTATACACTGGGGGACTATAACTTGCTTATT |
| | N25V, R42H, | | GATCCTAAAAAGATGCCACTGCTGCGCTTGC |
| | K48T, R54Y, | | GT |
| | S57G, A44T, | | |
| | S49L, P38L | | |
| CM147 | K6R, T7M, | MLIFVRMLTGK | ATGTTGATTTTCGTACGCATGTTGACTGGAAA |
| [562; 779] | T12M, T14E, | MIELEVMPSDTIE | GATGATCGAGTTGGAAGTGATGCCTTCCGAT |
| | K33H, A46Q, | VVKAKIQDHEGI | ACTATCGAGGTAGTTAAGGCCAAAATCCAAG |
| | S65P, L67K, | PLDQQHLTFQGT | ATCATGAAGGGATTCCTTTGGATCAACAACAT |
| | H68M, I61L, | LLEDGYTLGDYNL | CTTACTTTTCAAGGGACTTTGCTGGAGGACG |
| | K63I, E18M, | LIDPKKMPLLRLQ | GTTATACACTGGGGGACTATAACTTGCTTATT |
| | N25V, R42H, | | GATCCTAAAAAGATGCCACTGCTGCGCTTGC |
| | K48T, R54Y, | | AA |
| | S57G, A44T, | | |
| | S49L, P38L, | | |
| | R74Q | | |
| CM148 | K6R, T7M, | MLIFVRMLTGK | ATGTTGATTTTCGTACGCATGTTGACTGGAAA |
| [563; 780] | T12M, T14E, | MIELEVMPSDTIE | GATGATCGAGTTGGAAGTGATGCCTTCCGAT |
| | K33H, A46Q, | VVKAKIQDHEGI | ACTATCGAGGTAGTTAAGGCCAAAATCCAAG |
| | S65P, L67K, | PLDEQHLTFQGT | ATCATGAAGGGATTCCTTTGGATGAGCAACA |
| | H68M, I61L, | LLDDGYTLGDYN | TCTTACTTTTCAAGGGACTTTGCTGGATGACG |
| | K63I, E18M, | LLIDPKKMPLLRL | GTTATACACTGGGGGACTATAACTTGCTTATT |
| | N25V, R42H, | Q | GATCCTAAAAAGATGCCACTGCTGCGCTTGC |
| | K48T, R54Y, | | AA |
| | S57G, A44T, | | |
| | S49L, P38L, | | |
| | R74Q, N25V, | | |
| | Q40E, E51D | | |
| CM149 | K6R, T7M, | YLIFVRMLTGKMI | TATTTGATTTTCGTACGCATGTTGACTGGAAA |
| [564; 781] | T12M, T14E, | ELEVMPSDTIEVI | GATGATCGAGTTGGAAGTGATGCCTTCCGAT |
| | K33H, A46Q, | KAKIQDHEGIPLD | ACTATCGAGGTAATTAAGGCCAAAATCCAAG |
| | S65P, L67K, | EQHLTFQGTLLD | ATCATGAAGGGATTCCTTTGGATGAGCAACA |
| | H68M, I61L, | DGYTLGDYNLLID | TCTTACTTTTLAAGGGACTTTGCTGGATGALG |
| | K63I, E18M, | PKKMPLLRMQ | GTTATACACTGGGGGACTATAACTTGCTTATT |
| | N25V, R42H, | | GATCCTAAAAAGATGCCACTGCTGCGCATGC |
| | K48T, R54Y, | | AA |
| | S57G, A44T, | | |
| | S49L, P38L, | | |
| | R74Q, N25V, | | |
| | Q40E, E51D, | | |
| | M1Y, V26I, | | |
| | L73M | | |
| CM199 | K6R, T7M, | YLIFVRMLTGKMI | TATTTGATTTTCGTACGCATGTTGACTGGAAA |
| [565; 782] | T12M, T14E, | ELEVMPSDTIEVI | GATGATCGAGTTGGAAGTGATGCCTTCCGAT |
| | K33H, A46Q, | KAKIQDHEGIPP | ACTATCGAGGTAATTAAGGCCAAAATCCAAG |
| | S65P, L67K, | DEQRLAFQGTSL | ATCATGAAGGGATTCCTCCAGATGAGCAACG |
| | H68M, I61L, | DDGRTLGDYNLLI | CCTTGCTTTTCAAGGGACTAGCCTGGATGACG |
| | K63I, E18M, | DPKKMLVLRMQ | GTCGCACACTGGGGGACTATAACTTGCTTATT |
| | K48T, E51D, | | GATCCTAAAAAGATGTTGGTACTGCGCATGC |
| | S57G, M1Y, | | AA |
| | V26I, L73M, | | |
| | R74Q, N25V, | | |
| | Q40E, E51D, | | |
| | P69L, L70V | | |
| CM203 | K6R, T7M, | YLIFVRMLTGKMI | TATTTGATTTTCGTACGCATGTTGACTGGAAA |
| [566; 783] | T12M, T14E, | ELEVEPSDTIENIK | GATGATCGAGTTGGAAGTGGAGCCTTCCGAT |
| | K33H, A46Q, | AKIQDHEGIPLD | ACTATCGAGAATATTAAGGCCAAAATCCAAG |
| | S65P, L67K, | QQRLTFQGKLLE | ATCATGAAGGGATTCCTTTGGATCAACAACG |
| | H68M, I61L, | DGRTLSDYNLLID | CCTTACTTTTCAAGGGAAGTTGCTGGAGGAC |
| | K63I, A44T, | PKKMLVLRMQ | GGTCGCACACTGTCTGACTATAACTTGCTTAT |
| | S49L, P38L, | | TGATCCTAAAAAGATGTTGGTACTGCGCATG |
| | R74Q, M1Y, | | CAA |
| | V26I, L73M, | | |
| | P69L, L70V | | |
| CM204 | K6R, T7M, | MLIFVRMLTGK | ATGTTGATTTTCGTACGCATGTTGACTGGAAA |
| [567; 784] | T12M, T14E, | MIELEVEPSDTIE | GATGATCGAGTTGGAAGTGGAGCCTTCCGAT |
| | K33H, A46Q, | VVKAKIQDHEGI | ACTATCGAGGTAGTTAAGGCCAAAATCCAAG |
| | S65P, L67K, | PLDEQRLTFQGK | ATCATGAAGGGATTCCTTTGGATGAGCAACG |
| | H68M, I61L, | LLDDGRTLSDYNL | CCTTACTTTTCAAGGGAAGTTGCTGGATGACG |
| | K63I, A44T, | LIDPKKMLVLRLQ | GTCGCACACTGTCTGACTATAACTTGCTTATT |
| | S49L, P38L, | | GATCCTAAAAAGATGTTGGTACTGCGCTTGC |
| | R74Q, N25V, | | AA |
| | Q40E, E51D, | | |
| | P69L, L70V | | |
| CM208 | K6R, T7M, | MLIFVRMLTGK | ATGTTGATTTTCGTACGCATGTTGACTGGAAA |
| [568; 785] | T12M, T14E, | MIELEVMPSDTIE | GATGATCGAGTTGGAAGTGATGCCTTCCGAT |
| | K33H, A46Q, | VVKAKIQDHEGI | ACTATCGAGGTAGTTAAGGCCAAAATCCAAG |
| | S65P, L67K, | PLDQQHLTFQGT | ATCATGAAGGGATTCCTTTGGATCAACAACAT |
| | H68M, I61L, | LLEDGYTLGDYNL | CTTACTTTTCAAGGGACTTTGCTGGAGGACG |
| | K63I, E18M, | LIDPKKMLVLRLQ | GTTATACACTGGGGGACTATAACTTGCTTATT |
| | N25V, R42H, | | GATCCTAAAAAGATGTTGGTACTGCGCTTGC |
| | K48T, R54Y, | | AA |
| | S57G, A44T, | | |
| | S49L, P38L, | | |
| | R74Q, P69L, | | |
| | L70V | | |
| CM210 | K6R, T7M, | YLIFVRMLTGKMI | TATTTGATTTTCGTACGCATGTTGACTGGAAA |
| [569; 786] | T12M, T14E, | ELEVMPSDTIEVI | GATGATCGAGTTGGAAGTGATGCCTTCCGAT |
| | K33H, A46Q, | KAKIQDHEGIPLD | ACTATCGAGGTAATTAAGGCCAAAATCCAAG |
| | S65P, L67K, | EQHLTFQGTLLD | ATCATGAAGGGATTCCTTTGGATGAGCAACA |
| | H68M, I61L, | DGYTLGDYNLLID | TCTTACTTTTLAAGGGACTTTGCTGGATGALG |
| | N25V, R42H, | PKKMLVLRMQ | GTTATACACTGGGGGACTATAACTTGCTTATT |
| | K48T, R54Y, | | GATCCTAAAAAGATGTTGGTACTGCGCATGC |
| | S57G, A44T, | | AA |
| | S49L, P38L, | | |
| | R74Q, N25V, | | |
| | Q40E, E51D, | | |
| | M1Y, V26I, | | |
| | L73M, P69L, | | |
| | L70V | | |
| CM211 | K6R, T7M, | MLIFVRMLTGK | ATGTTGATTTTCGTACGCATGTTGACTGGAAA |
| [570; 787] | T12M, T14E, | MIELEVMPSDTIE | GATGATCGAGTTGGAAGTGATGCCTTCCGAT |
| | K33H, A46Q, | VVKAKIQDHEGI | ACTATCGAGGTAGTTAAGGCCAAAATCCAAG |
| | S65P, L67K, | PPDQQHLAFQG | ATCATGAAGGGATTCCTCCAGATCAACAACAT |
| | H68M, I61L, | TSLEDGYTLGDY | CTTGCTTTTCAAGGGACTAGCCTGGAGGACG |
| | K63I, E18M, | NLLIDPKKMLVLR | GTTATACACTGGGGGACTATAACTTGCTTATT |
| | N25V, R42H, | LR | GATCCTAAAAAGATGTTGGTACTGCGCTTGC |
| | K48T, R54Y, | | GT |
| | S57G, P69L, | | |
| | L70V | | |
| CM358 | K6R, T7M, | YLIFVRMLTGKMI | TATTTGATTTTCGTACGCATGTTGACTGGAAA |
| [571; 788] | T12M, T14E, | ELEVEPSDTIENIK | GATGATCGAGTTGGAAGTGGAGCCTTCCGAT |
| | K33H, A46Q, | AKIQDHEGIPLD | ACTATCGAGAATATTAAGGCCAAAATCCAAG |
| | S65P, L67K, | QQRLTFQGKLLE | ATCATGAAGGGATTCCTTTGGATCAACAACG |
| | H68M, I61L, | DGRTLSDYNLLID | CCTTACTTTTCAAGGGAAGTTGCTGGAGGAC |
| | K63I, A44T, | PKKMAVLRMQ | GGTCGCACACTGTCTGACTATAACTTGCTTAT |
| | S49L, P38L, | | TGATCCTAAAAAGATGGCCGTACTGCGCATG |
| | R74Q, M1Y, | | CAA |
| | V26I, L73M, | | |
| | P69A, L70V | | |
| CM359 | K6R, T7M, | YLIFVRMLTGKMI | TATTTGATTTTCGTACGCATGTTGACTGGAAA |
| [572; 789] | T12M, T14E, | ELEVEPSDTIENIK | GATGATCGAGTTGGAAGTGGAGCCTTCCGAT |
| | K33H, A46Q, | AKIQDHEGIPLD | ACTATCGAGAATATTAAGGCCAAAATCCAAG |
| | S65P, L67K, | QQRLTFQGKLLE | ATCATGAAGGGATTCCTTTGGATCAACAACG |
| | H68M, I61L, | DGRTLSDYNLLID | CCTTACTTTTCAAGGGAAGTTGCTGGAGGAC |
| | K63I, A44T, | PKKMRVLRMQ | GGTCGCACACTGTCTGACTATAACTTGCTTAT |
| | S49L, P38L, | | TGATCCTAAAAAGATGCGCGTACTGCGCATG |
| | R74Q, M1Y, | | CAA |
| | V26I, L73M, | | |
| | P69R, L70V | | |
| CM360 | K6R, T7M, | YLIFVRMLTGKMI | TATTTGATTTTCGTACGCATGTTGACTGGAAA |
| [573; 790] | T12M, T14E, | ELEVEPSDTIENIK | GATGATCGAGTTGGAAGTGGAGCCTTCCGAT |
| | K33H, A46Q, | AKIQDHEGIPLD | ACTATCGAGAATATTAAGGCCAAAATCCAAG |
| | S65P, L67K, | QQRLTFQGKLLE | ATCATGAAGGGATTCCTTTGGATCAACAACG |
| | H68M, I61L, | DGRTLSDYNLLID | CCTTACTTTTCAAGGGAAGTTGCTGGAGGAC |
| | K63I, A44T, | PKKMNVLRMQ | GGTCGCACACTGTCTGACTATAACTTGCTTAT |
| | S49L, P38L, | | TGATCCTAAAAAGATGAATGTACTGCGCATG |
| | R74Q, M1Y, | | CAA |
| | V26I, L73M, | | |
| | P69N, L70V | | |
| CM361 | K6R, T7M, | YLIFVRMLTGKMI | TATTTGATTTTCGTACGCATGTTGACTGGAAA |
| [574; 791] | T12M, T14E, | ELEVEPSDTIENIK | GATGATCGAGTTGGAAGTGGAGCCTTCCGAT |
| | K33H, A46Q, | AKIQDHEGIPLD | ACTATCGAGAATATTAAGGCCAAAATCCAAG |
| | S65P, L67K, | QQRLTFQGKLLE | ATCATGAAGGGATTCCTTTGGATCAACAACG |
| | H68M, I61L, | DGRTLSDYNLLID | CCTTACTTTTCAAGGGAAGTTGCTGGAGGAC |
| | K63I, A44T, | PKKMDVLRMQ | GGTCGCACACTGTCTGACTATAACTTGCTTAT |
| | S49L, P38L, | | TGATCCTAAAAAGATGGATGTACTGCGCATG |
| | R74Q, M1Y, | | CAA |
| | V26I, L73M, | | |
| | P69D, L70V | | |
| CM362 | K6R, T7M, | YLIFVRMLTGKMI | TATTTGATTTTCGTACGCATGTTGACTGGAAA |
| [575; 792] | T12M, T14E, | ELEVEPSDTIENIK | GATGATCGAGTTGGAAGTGGAGCCTTCCGAT |
| | K33H, A46Q, | AKIQDHEGIPLD | ACTATCGAGAATATTAAGGCCAAAATCCAAG |
| | S65P, L67K, | QQRLTFQGKLLE | ATCATGAAGGGATTCCTTTGGATCAACAACG |
| | H68M, I61L, | DGRTLSDYNLLID | CCTTACTTTTCAAGGGAAGTTGCTGGAGGAC |
| | K63I, A44T, | PKKMCVLRMQ | GGTCGCACACTGTCTGACTATAACTTGCTTAT |
| | S49L, P38L, | | TGATCCTAAAAAGATGTGCGTACTGCGCATG |
| | R74Q, M1Y, | | CAA |
| | V26I, L73M, | | |
| | P69C, L70V | | |
| CM363 | K6R, T7M, | YLIFVRMLTGKMI | TATTTGATTTTCGTACGCATGTTGACTGGAAA |
| [576; 793] | T12M, T14E, | ELEVEPSDTIENIK | GATGATCGAGTTGGAAGTGGAGCCTTCCGAT |
| | K33H, A46Q, | AKIQDHEGIPLD | ACTATCGAGAATATTAAGGCCAAAATCCAAG |
| | S65P, L67K, | QQRLTFQGKLLE | ATCATGAAGGGATTCCTTTGGATCAACAACG |
| | H68M, I61L, | DGRTLSDYNLLID | CLTTACTTTTCAAGGGAAGTTGCTGGAGGAC |
| | K63I, A44T, | PKKMEVLRMQ | GGTCGCACACTGTCTGACTATAACTTGCTTAT |
| | S49L, P38L, | | TGATCCTAAAAAGATGGAGGTACTGCGCATG |
| | R74Q, M1Y, | | CAA |
| | V26I, L73M, | | |
| | P69E, L70V | | |
| CM364 | K6R, T7M, | YLIFVRMLTGKMI | TATTTGATTTTCGTACGCATGTTGACTGGAAA |
| [577; 794] | T12M, T14E, | ELEVEPSDTIENIK | GATGATCGAGTTGGAAGTGGAGCCTTCCGAT |
| | K33H, A46Q, | AKIQDHEGIPLD | ACTATCGAGAATATTAAGGCCAAAATCCAAG |
| | S65P, L67K, | QQRLTFQGKLLE | ATCATGAAGGGATTCCTTTGGATCAACAACG |
| | H68M, I61L, | DGRTLSDYNLLID | CCTTACTTTTCAAGGGAAGTTGCTGGAGGAC |
| | K63I, A44T, | PKKMQVLRMQ | GGTCGCACACTGTCTGACTATAACTTGCTTAT |
| | S49L, P38L, | | TGATCCTAAAAAGATGCAAGTACTGCGCATG |
| | R74Q, M1Y, | | CAA |
| | V26I, L73M, | | |
| | P690, L70V | | |
| CM365 | K6R, T7M, | YLIFVRMLTGKMI | TATTTGATTTTCGTACGCATGTTGACTGGAAA |
| [578; 795] | T12M, T14E, | ELEVEPSDTIENIK | GATGATCGAGTTGGAAGTGGAGCCTTCCGAT |
| | K33H, A46Q, | AKIQDHEGIPLD | ACTATCGAGAATATTAAGGCCAAAATCCAAG |
| | S65P, L67K, | QQRLTFQGKLLE | ATCATGAAGGGATTCCTTTGGATCAACAACG |
| | H68M, I61L, | DGRTLSDYNLLID | CCTTACTTTTCAAGGGAAGTTGCTGGAGGAC |
| | K63I, A44T, | PKKMGVLRMQ | GGTCGCACACTGTCTGACTATAACTTGCTTAT |
| | S49L, P38L, | | TGATCCTAAAAAGATGGGGGTACTGCGCATG |
| | R74Q, M1Y, | | CAA |
| | V26I, L73M, | | |
| | P69G, L70V | | |
| CM366 | K6R, T7M, | YLIFVRMLTGKMI | TATTTGATTTTCGTACGCATGTTGACTGGAAA |
| [579; 796] | T12M, T14E, | ELEVEPSDTIENIK | GATGATCGAGTTGGAAGTGGAGCCTTCCGAT |
| | K33H, A46Q, | AKIQDHEGIPLD | ACTATCGAGAATATTAAGGCCAAAATCCAAG |
| | S65P, L67K, | QQRLTFQGKLLE | ATCATGAAGGGATTCCTTTGGATCAACAACG |
| | H68M, I61L, | DGRTLSDYNLLID | CLTTACTTTTCAAGGGAAGTTGCTGGAGGAC |
| | K63I, A44T, | PKKMHVLRMQ | GGTCGCACACTGTCTGACTATAACTTGCTTAT |
| | S49L, P38L, | | TGATCCTAAAAAGATGCATGTACTGCGCATG |
| | R74Q, M1Y, | | CAA |
| | V26I, L73M, | | |
| | P69H, L70V | | |
| CM367 | K6R, T7M, | YLIFVRMLTGKMI | TATTTGATTTTCGTACGCATGTTGACTGGAAA |
| [580; 797] | T12M, T14E, | ELEVEPSDTIENIK | GATGATCGAGTTGGAAGTGGAGCCTTCCGAT |
| | K33H, A46Q, | AKIQDHEGIPLD | ACTATCGAGAATATTAAGGCCAAAATCCAAG |
| | S65P, L67K, | QQRLTFQGKLLE | ATCATGAAGGGATTCCTTTGGATCAACAACG |
| | H68M, I61L, | DGRTLSDYNLLID | CCTTACTTTTCAAGGGAAGTTGCTGGAGGAC |
| | K63I, A44T, | PKKMIVLRMQ | GGTCGCACACTGTCTGACTATAACTTGCTTAT |
| | S49L, P38L, | | TGATCCTAAAAAGATGATTGTACTGCGCATGC |
| | R74Q, M1Y, | | AA |
| | V26I, L73M, | | |
| | P69I, L70V | | |
| CM368 | K6R, T7M, | YLIFVRMLTGKMI | TATTTGATTTTCGTACGCATGTTGACTGGAAA |
| [581; 798] | T12M, T14E, | ELEVEPSDTIENIK | GATGATCGAGTTGGAAGTGGAGCCTTCCGAT |
| | K33H, A46Q, | AKIQDHEGIPLD | ACTATCGAGAATATTAAGGCCAAAATCCAAG |
| | S65P, L67K, | QQRLTFQGKLLE | ATCATGAAGGGATTCCTTTGGATCAACAACG |
| | H68M, I61L, | DGRTLSDYNLLID | CCTTACTTTTCAAGGGAAGTTGCTGGAGGAC |
| | K63I, A44T, | PKKMKVLRMQ | GGTCGCACACTGTCTGACTATAACTTGCTTAT |
| | S49L, P38L, | | TGATCCTAAAAAGATGAAGGTACTGCGCATG |
| | R74Q, M1Y, | | CAA |
| | V26I, L73M, | | |
| | P69K, L70V | | |
| CM369 | K6R, T7M, | YLIFVRMLTGKMI | TATTTGATTTTCGTACGCATGTTGACTGGAAA |
| [582; 799] | T12M, T14E, | ELEVEPSDTIENIK | GATGATCGAGTTGGAAGTGGAGCCTTCCGAT |
| | K33H, A46Q, | AKIQDHEGIPLD | ACTATCGAGAATATTAAGGCCAAAATCCAAG |
| | S65P, L67K, | QQRLTFQGKLLE | ATCATGAAGGGATTCCTTTGGATCAACAACG |
| | Hb8M, I61L, | DGRTLSDYNLLID | CCTTACTTTTLAAGGGAAGTTGCTGGAGGAC |
| | K63I, A44T, | PKKMMVLRMQ | GGTCGCACACTGTCTGACTATAACTTGCTTAT |
| | S49L, P38L, | | TGATCCTAAAAAGATGATGGTACTGCGCATG |
| | R74Q, M1Y, | | CAA |
| | V26I, L73M, | | |
| | P69M, L70V | | |
| CM370 | K6R, T7M, | YLIFVRMLTGKMI | TATTTGATTTTCGTACGCATGTTGACTGGAAA |
| [583; 800] | T12M, T14E, | ELEVEPSDTIENIK | GATGATCGAGTTGGAAGTGGAGCCTTCCGAT |
| | K33H, A46Q, | AKIQDHEGIPLD | ACTATCGAGAATATTAAGGCCAAAATCCAAG |
| | S65P, L67K, | QQRLTFQGKLLE | ATCATGAAGGGATTCCTTTGGATCAACAACG |
| | Hb8M, I61L, | DGRTLSDYNLLID | CCTTACTTTTLAAGGGAAGTTGCTGGAGGAC |
| | K63I, A44T, | PKKMFVLRMQ | GGTCGCACACTGTCTGACTATAACTTGCTTAT |
| | S49L, P38L, | | TGATCCTAAAAAGATGTTCGTACTGCGCATGC |
| | R74Q, M1Y, | | AA |
| | V26I, L73M, | | |
| | P69F, L70V | | |
| CM371 | K6R, T7M, | YLIFVRMLTGKMI | TATTTGATTTTCGTACGCATGTTGACTGGAAA |
| [584; 801] | T12M, T14E, | ELEVEPSDTIENIK | GATGATCGAGTTGGAAGTGGAGCCTTCCGAT |
| | K33H, A46Q, | AKIQDHEGIPLD | ACTATCGAGAATATTAAGGCCAAAATCCAAG |
| | S65P, L67K, | QQRLTFQGKLLE | ATCATGAAGGGATTCCTTTGGATCAACAACG |
| | H68M, I61L, | DGRTLSDYNLLID | CCTTACTTTTCAAGGGAAGTTGCTGGAGGAC |
| | K63I, A44T, | PKKMSVLRMQ | GGTCGCACACTGTCTGACTATAACTTGCTTAT |
| | S49L, P38L, | | TGATCCTAAAAAGATGTCTGTACTGCGCATGC |
| | R74Q, M1Y, | | AA |
| | V26I, L73M, | | |
| | P69S, L70V | | |
| CM372 | K6R, T7M, | YLIFVRMLTGKMI | TATTTGATTTTCGTACGCATGTTGACTGGAAA |
| [585; 802] | T12M, T14E, | ELEVEPSDTIENIK | GATGATCGAGTTGGAAGTGGAGCCTTCCGAT |
| | K33H, A46Q, | AKIQDHEGIPLD | ACTATCGAGAATATTAAGGCCAAAATCCAAG |
| | S65P, L67K, | QQRLTFQGKLLE | ATCATGAAGGGATTCCTTTGGATCAACAACG |
| | H68M, I61L, | DGRTLSDYNLLID | CCTTACTTTTCAAGGGAAGTTGCTGGAGGAC |
| | K63I, A44T, | PKKMTVLRMQ | GGTCGCACACTGTCTGACTATAACTTGCTTAT |
| | S49L, P38L, | | TGATCCTAAAAAGATGACTGTACTGCGCATG |
| | R74Q, M1Y, | | CAA |
| | V26I, L73M, | | |
| | P69T, L70V | | |
| CM373 | K6R, T7M, | YLIFVRMLTGKMI | TATTTGATTTTCGTACGCATGTTGACTGGAAA |
| [586; 803] | T12M, T14E, | ELEVEPSDTIENIK | GATGATCGAGTTGGAAGTGGAGCCTTCCGAT |
| | K33H, A46Q, | AKIQDHEGIPLD | ACTATCGAGAATATTAAGGCCAAAATCCAAG |
| | S65P, L67K, | QQRLTFQGKLLE | ATCATGAAGGGATTCCTTTGGATCAACAACG |
| | H68M, I61L, | DGRTLSDYNLLID | CCTTACTTTTLAAGGGAAGTTGCTGGAGGAC |
| | K63I, A44T, | PKKMWVLRMQ | GGTCGCACACTGTCTGACTATAACTTGCTTAT |
| | S49L, P38L, | | TGATCCTAAAAAGATGTGGGTACTGCGCATG |
| | R74Q, M1Y, | | CAA |
| | V26I, L73M, | | |
| | P69W, L70V | | |
| CM374 | K6R, T7M, | YLIFVRMLTGKMI | TATTTGATTTTCGTACGCATGTTGACTGGAAA |
| [587; 804] | T12M, T14E, | ELEVEPSDTIENIK | GATGATCGAGTTGGAAGTGGAGCCTTCCGAT |
| | K33H, A46Q, | AKIQDHEGIPLD | ACTATCGAGAATATTAAGGCCAAAATCCAAG |
| | S65P, L67K, | QQRLTFQGKLLE | ATCATGAAGGGATTCCTTTGGATCAACAACG |
| | H68M, I61L, | DGRTLSDYNLLID | CCTTACTTTTCAAGGGAAGTTGCTGGAGGAC |
| | K63I, A44T, | PKKMYVLRMQ | GGTCGCACACTGTCTGACTATAACTTGCTTAT |
| | S49L, P38L, | | TGATCCTAAAAAGATGTATGTACTGCGCATGC |
| | R74Q, M1Y, | | AA |
| | V26I, L73M, | | |
| | P69Y, L70V | | |
| CM375 | K6R, T7M, | YLIFVRMLTGKMI | TATTTGATTTTCGTACGCATGTTGACTGGAAA |
| [588; 805] | T12M, T14E, | ELEVEPSDTIENIK | GATGATCGAGTTGGAAGTGGAGCCTTCCGAT |
| | K33H, A46Q, | AKIQDHEGIPLD | ACTATCGAGAATATTAAGGCCAAAATCCAAG |
| | S65P, L67K, | QQRLTFQGKLLE | ATCATGAAGGGATTCCTTTGGATCAACAACG |
| | H68M, I61L, | DGRTLSDYNLLID | CCTTACTTTTCAAGGGAAGTTGCTGGAGGAC |
| | K63I, A44T, | PKKMVVLRMQ | GGTCGCACACTGTCTGACTATAACTTGCTTAT |
| | S49L, P38L, | | TGATCCTAAAAAGATGGTAGTACTGCGCATG |
| | R74Q, M1Y, | | CAA |
| | V26I, L73M, | | |
| | P69V, L70V | | |
| CM376 | K6R, T7M, | YLIFVRMLTGKMI | TATTTGATTTTCGTACGCATGTTGACTGGAAA |
| [589; 806] | T12M, T14E, | ELEVEPSDTIENIK | GATGATCGAGTTGGAAGTGGAGCCTTCCGAT |
| | K33H, A46Q, | AKIQDHEGIPLD | ACTATCGAGAATATTAAGGCCAAAATCCAAG |
| | S65P, L67K, | QQRLTFQGKLLE | ATCATGAAGGGATTCCTTTGGATCAACAACG |
| | H68M, I61L, | DGRTLSDYNLLID | CLTTACTTTTCAAGGGAAGTTGCTGGAGGAC |
| | K63I, A44T, | PKKMLALRMQ | GGTCGCACACTGTCTGACTATAACTTGCTTAT |
| | S49L, P38L, | | TGATCCTAAAAAGATGTTGGCCCTGCGCATG |
| | R74Q, M1Y, | | CAA |
| | V26I, L73M, | | |
| | P69L, L70A | | |
| CM377 | K6R, T7M, | YLIFVRMLTGKMI | TATTTGATTTTCGTACGCATGTTGACTGGAAA |
| [590; 807] | T12M, T14E, | ELEVEPSDTIENIK | GATGATCGAGTTGGAAGTGGAGCCTTCCGAT |
| | K33H, A46Q, | AKIQDHEGIPLD | ACTATCGAGAATATTAAGGCCAAAATCCAAG |
| | S65P, L67K, | QQRLTFQGKLLE | ATCATGAAGGGATTCCTTTGGATCAACAACG |
| | H68M, I61L, | DGRTLSDYNLLID | CCTTACTTTTCAAGGGAAGTTGCTGGAGGAC |
| | K63I, A44T, | PKKMLRLRMQ | GGTCGCACACTGTCTGACTATAACTTGCTTAT |
| | S49L, P38L, | | TGATCCTAAAAAGATGTTGCGCCTGCGCATG |
| | R74Q, M1Y, | | CAA |
| | V26I, L73M, | | |
| | P69L, L70R | | |
| CM378 | K6R, T7M, | YLIFVRMLTGKMI | TATTTGATTTTCGTACGCATGTTGACTGGAAA |
| [591; 808] | T12M, T14E, | ELEVEPSDTIENIK | GATGATCGAGTTGGAAGTGGAGCCTTCCGAT |
| | K33H, A46Q, | AKIQDHEGIPLD | ACTATCGAGAATATTAAGGCCAAAATCCAAG |
| | S65P, L67K, | QQRLTFQGKLLE | ATCATGAAGGGATTCCTTTGGATCAACAACG |
| | H68M, I61L, | DGRTLSDYNLLID | CCTTACTTTTCAAGGGAAGTTGCTGGAGGAC |
| | K63I, A44T, | PKKMLNLRMQ | GGTCGCACACTGTCTGACTATAACTTGCTTAT |
| | S49L, P38L, | | TGATCCTAAAAAGATGTTGAATCTGCGCATGC |
| | R74Q, M1Y, | | AA |
| | V26I, L73M, | | |
| | P69L, L70N | | |
| CM379 | K6R, T7M, | YLIFVRMLTGKMI | TATTTGATTTTCGTACGCATGTTGACTGGAAA |
| [592; 809] | T12M, T14E, | ELEVEPSDTIENIK | GATGATCGAGTTGGAAGTGGAGCCTTCCGAT |
| | K33H, A46Q, | AKIQDHEGIPLD | ACTATCGAGAATATTAAGGCCAAAATCCAAG |
| | S65P, L67K, | QQRLTFQGKLLE | ATCATGAAGGGATTCCTTTGGATCAACAACG |
| | H68M, I61L, | DGRTLSDYNLLID | CLTTACTTTTCAAGGGAAGTTGCTGGAGGAC |
| | K63I, A44T, | PKKMLDLRMQ | GGTCGCACACTGTCTGACTATAACTTGCTTAT |
| | S49L, P38L, | | TGATCCTAAAAAGATGTTGGATCTGCGCATG |
| | R74Q, M1Y, | | CAA |
| | V26I, L73M, | | |
| | P69L, L70D | | |
| CM380 | K6R, T7M, | YLIFVRMLTGKMI | TATTTGATTTTCGTACGCATGTTGACTGGAAA |
| [593; 810] | T12M, T14E, | ELEVEPSDTIENIK | GATGATCGAGTTGGAAGTGGAGCCTTCCGAT |
| | K33H, A46Q, | AKIQDHEGIPLD | ACTATCGAGAATATTAAGGCCAAAATCCAAG |
| | S65P, L67K, | QQRLTFQGKLLE | ATCATGAAGGGATTCCTTTGGATCAACAACG |
| | H68M, I61L, | DGRTLSDYNLLID | CCTTACTTTTCAAGGGAAGTTGCTGGAGGAC |
| | K63I, A44T, | PKKMLCLRMQ | GGTCGCACACTGTCTGACTATAACTTGCTTAT |
| | S49L, P38L, | | TGATCCTAAAAAGATGTTGTGCCTGCGCATGC |
| | R74Q, M1Y, | | AA |
| | V26I, L73M, | | |
| | P69L, L70C | | |
| CM381 | K6R, T7M, | YLIFVRMLTGKMI | TATTTGATTTTCGTACGCATGTTGACTGGAAA |
| [594; 811] | T12M, T14E, | ELEVEPSDTIENIK | GATGATCGAGTTGGAAGTGGAGCCTTCCGAT |
| | K33H, A46Q, | AKIQDHEGIPLD | ACTATCGAGAATATTAAGGCCAAAATCCAAG |
| | S65P, L67K, | QQRLTFQGKLLE | ATCATGAAGGGATTCCTTTGGATCAACAACG |
| | H68M, I61L, | DGRTLSDYNLLID | CCTTACTTTTCAAGGGAAGTTGCTGGAGGAC |
| | K63I, A44T, | PKKMLELRMQ | GGTCGCACACTGTCTGACTATAACTTGCTTAT |
| | S49L, P38L, | | TGATCCTAAAAAGATGTTGGAGCTGCGCATG |
| | R74Q, M1Y, | | CAA |
| | V26I, L73M, | | |
| | P69L, L70E | | |
| CM382 | K6R, T7M, | YLIFVRMLTGKMI | TATTTGATTTTCGTACGCATGTTGACTGGAAA |
| [595; 812] | T12M, T14E, | ELEVEPSDTIENIK | GATGATCGAGTTGGAAGTGGAGCCTTCCGAT |
| | K33H, A46Q, | AKIQDHEGIPLD | ACTATCGAGAATATTAAGGCCAAAATCCAAG |
| | S65P, L67K, | QQRLTFQGKLLE | ATCATGAAGGGATTCCTTTGGATCAACAACG |
| | H68M, I61L, | DGRTLSDYNLLID | CCTTACTTTTCAAGGGAAGTTGCTGGAGGAC |
| | K63I, A44T, | PKKMLQLRMQ | GGTCGCACACTGTCTGACTATAACTTGCTTAT |
| | S49L, P38L, | | TGATCCTAAAAAGATGTTGCAACTGCGCATG |
| | R74Q, M1Y, | | CAA |
| | V26I, L73M, | | |
| | P69L, L700 | | |
| CM383 | K6R, T7M, | YLIFVRMLTGKMI | TATTTGATTTTCGTACGCATGTTGACTGGAAA |
| [596; 813] | T12M, T14E, | ELEVEPSDTIENIK | GATGATCGAGTTGGAAGTGGAGCCTTCCGAT |
| | K33H, A46Q, | AKIQDHEGIPLD | ACTATCGAGAATATTAAGGCCAAAATCCAAG |
| | S65P, L67K, | QQRLTFQGKLLE | ATCATGAAGGGATTCCTTTGGATCAACAACG |
| | H68M, I61L, | DGRTLSDYNLLID | CCTTACTTTTCAAGGGAAGTTGCTGGAGGAC |
| | K63I, A44T, | PKKMLGLRMQ | GGTCGCACACTGTCTGACTATAACTTGCTTAT |
| | S49L, P38L, | | TGATCCTAAAAAGATGTTGGGGCTGCGCATG |
| | R74Q, M1Y, | | CAA |
| | V26I, L73M, | | |
| | P69L, L70G | | |
| CM384 | K6R, T7M, | YLIFVRMLTGKMI | TATTTGATTTTCGTACGCATGTTGACTGGAAA |
| [597; 814] | T12M, T14E, | ELEVEPSDTIENIK | GATGATCGAGTTGGAAGTGGAGCCTTCCGAT |
| | K33H, A46Q, | AKIQDHEGIPLD | ACTATCGAGAATATTAAGGCCAAAATCCAAG |
| | S65P, L67K, | QQRLTFQGKLLE | ATCATGAAGGGATTCCTTTGGATCAACAACG |
| | H68M, I61L, | DGRTLSDYNLLID | CCTTACTTTTCAAGGGAAGTTGCTGGAGGAC |
| | K63I, A44T, | PKKMLHLRMQ | GGTCGCACACTGTCTGACTATAACTTGCTTAT |
| | S49L, P38L, | | TGATCCTAAAAAGATGTTGCATCTGCGCATGC |
| | R74Q, M1Y, | | AA |
| | V26I, L73M, | | |
| | P69L, L70H | | |
| CM385 | K6R, T7M, | YLIFVRMLTGKMI | TATTTGATTTTCGTACGCATGTTGACTGGAAA |
| [598; 815] | T12M, T14E, | ELEVEPSDTIENIK | GATGATCGAGTTGGAAGTGGAGCCTTCCGAT |
| | K33H, A46Q, | AKIQDHEGIPLD | ACTATCGAGAATATTAAGGCCAAAATCCAAG |
| | S65P, L67K, | QQRLTFQGKLLE | ATCATGAAGGGATTCCTTTGGATCAACAACG |
| | H68M, I61L, | DGRTLSDYNLLID | CCTTACTTTTCAAGGGAAGTTGCTGGAGGAC |
| | K63I, A44T, | PKKMLILRMQ | GGTCGCACACTGTCTGACTATAACTTGCTTAT |
| | S49L, P38L, | | TGATCCTAAAAAGATGTTGATTCTGCGCATGC |
| | R74Q, M1Y, | | AA |
| | V26I, L73M, | | |
| | P69L, L70I | | |
| CM386 | K6R, T7M, | YLIFVRMLTGKMI | TATTTGATTTTCGTACGCATGTTGACTGGAAA |
| [599; 816] | T12M, T14E, | ELEVEPSDTIENIK | GATGATCGAGTTGGAAGTGGAGCCTTCCGAT |
| | K33H, A46Q, | AKIQDHEGIPLD | ACTATCGAGAATATTAAGGCCAAAATCCAAG |
| | S65P, L67K, | QQRLTFQGKLLE | ATCATGAAGGGATTCCTTTGGATCAACAACG |
| | H68M, I61L, | DGRTLSDYNLLID | CLTTACTTTTCAAGGGAAGTTGCTGGAGGAC |
| | K63I, A44T, | PKKMLKLRMQ | GGTCGCACACTGTCTGACTATAACTTGCTTAT |
| | S49L, P38L, | | TGATCCTAAAAAGATGTTGAAGCTGCGCATG |
| | R74Q, M1Y, | | CAA |
| | V26I, L73M, | | |
| | P69L, L70K | | |
| CM387 | K6R, T7M, | YLIFVRMLTGKMI | TATTTGATTTTCGTACGCATGTTGACTGGAAA |
| [600; 817] | T12M, T14E, | ELEVEPSDTIENIK | GATGATCGAGTTGGAAGTGGAGCCTTCCGAT |
| | K33H, A46Q, | AKIQDHEGIPLD | ACTATCGAGAATATTAAGGCCAAAATCCAAG |
| | S65P, L67K, | QQRLTFQGKLLE | ATCATGAAGGGATTCCTTTGGATCAACAACG |
| | H68M, I61L, | DGRTLSDYNLLID | CCTTACTTTTCAAGGGAAGTTGCTGGAGGAC |
| | K63I, A44T, | PKKMLMLRMQ | GGTCGCACACTGTCTGACTATAACTTGCTTAT |
| | S49L, P38L, | | TGATCCTAAAAAGATGTTGATGCTGCGCATG |
| | R74Q, M1Y, | | CAA |
| | V26I, L73M, | | |
| | P69L, L70M | | |
| CM388 | K6R, T7M, | YLIFVRMLTGKMI | TATTTGATTTTCGTACGCATGTTGACTGGAAA |
| [601; 818] | T12M, T14E, | ELEVEPSDTIENIK | GATGATCGAGTTGGAAGTGGAGCCTTCCGAT |
| | K33H, A46Q, | AKIQDHEGIPLD | ACTATCGAGAATATTAAGGCCAAAATCCAAG |
| | S65P, L67K, | QQRLTFQGKLLE | ATCATGAAGGGATTCCTTTGGATCAACAACG |
| | H68M, I61L, | DGRTLSDYNLLID | CCTTACTTTTCAAGGGAAGTTGCTGGAGGAC |
| | K63I, A44T, | PKKMLFLRMQ | GGTCGCACACTGTCTGACTATAACTTGCTTAT |
| | S49L, P38L, | | TGATCCTAAAAAGATGTTGTTCCTGCGCATGC |
| | R74Q, M1Y, | | AA |
| | V26I, L73M, | | |
| | P69L, L70F | | |
| CM389 | K6R, T7M, | YLIFVRMLTGKMI | TATTTGATTTTCGTACGCATGTTGACTGGAAA |
| [602; 819] | T12M, T14E, | ELEVEPSDTIENIK | GATGATCGAGTTGGAAGTGGAGCCTTCCGAT |
| | K33H, A46Q, | AKIQDHEGIPLD | ACTATCGAGAATATTAAGGCCAAAATCCAAG |
| | S65P, L67K, | QQRLTFQGKLLE | ATCATGAAGGGATTCCTTTGGATCAACAACG |
| | H68M, I61L, | DGRTLSDYNLLID | CLTTACTTTTCAAGGGAAGTTGCTGGAGGAC |
| | K63I, A44T, | PKKMLPLRMQ | GGTCGCACACTGTCTGACTATAACTTGCTTAT |
| | S49L, P38L, | | TGATCCTAAAAAGATGTTGCCTCTGCGCATGC |
| | R74Q, M1Y, | | AA |
| | V26I, L73M, | | |
| | P69L, L70P | | |
| CM390 | K6R, T7M, | YLIFVRMLTGKMI | TATTTGATTTTCGTACGCATGTTGACTGGAAA |
| [603; 820] | T12M, T14E, | ELEVEPSDTIENIK | GATGATCGAGTTGGAAGTGGAGCCTTCCGAT |
| | K33H, A46Q, | AKIQDHEGIPLD | ACTATCGAGAATATTAAGGCCAAAATCCAAG |
| | S65P, L67K, | QQRLTFQGKLLE | ATCATGAAGGGATTCCTTTGGATCAACAACG |
| | H68M, I61L, | DGRTLSDYNLLID | CCTTACTTTTCAAGGGAAGTTGCTGGAGGAC |
| | K63I, A44T, | PKKMLSLRMQ | GGTCGCACACTGTCTGACTATAACTTGCTTAT |
| | S49L, P38L, | | TGATCCTAAAAAGATGTTGTCTCTGCGCATGC |
| | R74Q, M1Y, | | AA |
| | V26I, L73M, | | |
| | P69L, L70S | | |
| CM391 | K6R, T7M, | YLIFVRMLTGKMI | TATTTGATTTTCGTACGCATGTTGACTGGAAA |
| [604; 821] | T12M, T14E, | ELEVEPSDTIENIK | GATGATCGAGTTGGAAGTGGAGCCTTCCGAT |
| | K33H, A46Q, | AKIQDHEGIPLD | ACTATCGAGAATATTAAGGCCAAAATCCAAG |
| | S65P, L67K, | QQRLTFQGKLLE | ATCATGAAGGGATTCCTTTGGATCAACAACG |
| | H68M, I61L, | DGRTLSDYNLLID | CCTTACTTTTCAAGGGAAGTTGCTGGAGGAC |
| | K63I, A44T, | PKKMLTLRMQ | GGTCGCACACTGTCTGACTATAACTTGCTTAT |
| | S49L, P38L, | | TGATCCTAAAAAGATGTTGACTCTGCGCATGC |
| | R74Q, M1Y, | | AA |
| | V26I, L73M, | | |
| | P69L, L70T | | |
| CM392 | K6R, T7M, | YLIFVRMLTGKMI | TATTTGATTTTCGTACGCATGTTGACTGGAAA |
| [605; 822] | T12M, T14E, | ELEVEPSDTIENIK | GATGATCGAGTTGGAAGTGGAGCCTTCCGAT |
| | K33H, A46Q, | AKIQDHEGIPLD | ACTATCGAGAATATTAAGGCCAAAATCCAAG |
| | S65P, L67K, | QQRLTFQGKLLE | ATCATGAAGGGATTCCTTTGGATCAACAACG |
| | H68M, I61L, | DGRTLSDYNLLID | CCTTACTTTTLAAGGGAAGTTGCTGGAGGAC |
| | K63I, A44T, | PKKMLWLRMQ | GGTCGCACACTGTCTGACTATAACTTGCTTAT |
| | S49L, P38L, | | TGATCCTAAAAAGATGTTGTGGCTGCGCATG |
| | R74Q, M1Y, | | CAA |
| | V26I, L73M, | | |
| | P69L, L70W | | |
| CM393 | K6R, T7M, | YLIFVRMLTGKMI | TATTTGATTTTCGTACGCATGTTGACTGGAAA |
| [606; 823] | T12M, T14E, | ELEVEPSDTIENIK | GATGATCGAGTTGGAAGTGGAGCCTTCCGAT |
| | K33H, A46Q, | AKIQDHEGIPLD | ACTATCGAGAATATTAAGGCCAAAATCCAAG |
| | S65P, L67K, | QQRLTFQGKLLE | ATCATGAAGGGATTCCTTTGGATCAACAACG |
| | H68M, I61L, | DGRTLSDYNLLID | CCTTACTTTTLAAGGGAAGTTGCTGGAGGAC |
| | K63I, A44T, | PKKMLYLRMQ | GGTCGCACACTGTCTGACTATAACTTGCTTAT |
| | S49L, P38L, | | TGATCCTAAAAAGATGTTGTATCTGCGCATGC |
| | R74Q, M1Y, | | AA |
| | V26I, L73M, | | |
| | P69L, L70Y | | |
| CM429 | K6R, T7M, | YMIFVRMLTGK | TATATGATTTTCGTACGCATGTTGACTGGAAA |
| [607; 824] | T12M, T14E, | MIELEVEPSDTIE | GATGATCGAGTTGGAAGTGGAGCCTTCCGAT |
| | K33H, A46Q, | NIKAKIQDHEGIP | ACTATCGAGAATATTAAGGCCAAAATCCAAG |
| | S65P, L67K, | LDQQRLTFQGKL | ATCATGAAGGGATTCCTTTGGATCAACAACG |
| | H68M, I61L, | LEDGRTLSDYNLP | CCTTACTTTTCAAGGGAAGTTGCTGGAGGAC |
| | K63I, A44T, | ISPEKMAVLRMQ | GGTCGCACACTGTCTGACTATAACTTGCCTAT |
| | S49L, P38L, | | TTCTCCTGAGAAGATGGCCGTACTGCGCATG |
| | R74Q, M1Y, | | CAA |
| | V26I, L73M, | | |
| | P69A, L70V, | | |
| | L2M, L62P, | | |
| | D64S, K66E | | |
| CM430 | K6R, T7M, | YMIFVRMLTGK | TATATGATTTTCGTACGCATGTTGACTGGAAA |
| [608; 825] | T12M, T14E, | MIELEVEPSDTIE | GATGATCGAGTTGGAAGTGGAGCCTTCCGAT |
| | K33H, A46Q, | NIKAKIQDHEGIP | ACTATCGAGAATATTAAGGCCAAAATCCAAG |
| | S65P, L67K, | LDQQRLTFQGKL | ATCATGAAGGGATTCCTTTGGATCAACAACG |
| | H68M, I61L, | LEDGRTLSDYNLP | CCTTACTTTTCAAGGGAAGTTGCTGGAGGAC |
| | K63I, A44T, | ISPEKMGVLRMQ | GGTCGCACACTGTCTGACTATAACTTGCCTAT |
| | S49L, P38L, | | TTCTCCTGAGAAGATGGGGGTACTGCGCATG |
| | R74Q, M1Y, | | CAA |
| | V26I, L73M, | | |
| | P69G, L70V, | | |
| | L2M, L62P, | | |
| | D64S, K66E | | |
| CM431 | K6R, T7M, | YMIFVRMLTGK | TATATGATTTTCGTACGCATGTTGACTGGAAA |
| [609; 826] | T12M, T14E, | MIELEVEPSDTIE | GATGATCGAGTTGGAAGTGGAGCCTTCCGAT |
| | K33H, A46Q, | NIKAKIQDHEGIP | ACTATCGAGAATATTAAGGCCAAAATCCAAG |
| | S65P, L67K, | LDQQRLTFQGKL | ATCATGAAGGGATTCCTTTGGATCAACAACG |
| | H68M, I61L, | LEDGRTLSDYNLP | CCTTACTTTTLAAGGGAAGTTGCTGGAGGAC |
| | K63I, A44T, | ISPEKMLMLRM | GGTCGCACACTGTCTGACTATAACTTGCCTAT |
| | S49L, P38L, | Q | TTCTCCTGAGAAGATGTTGATGCTGCGCATGC |
| | R74Q, M1Y, | | AA |
| | V26I, L73M, | | |
| | P69L, L70M, | | |
| | L2M, L62P, | | |
| | D64S, K66E | | |
| CM432 | K6R, T7M, | YMIFVRMLTGK | TATATGATTTTCGTACGCATGTTGACTGGAAA |
| [610; 827] | T12M, T14E, | MIELEVEPSDTIE | GATGATCGAGTTGGAAGTGGAGCCTTCCGAT |
| | K33H, A46Q, | NIKAKIQDHEGIP | ACTATCGAGAATATTAAGGCCAAAATCCAAG |
| | S65P, L67K, | LDQQRLTFQGKL | ATCATGAAGGGATTCCTTTGGATCAACAACG |
| | H68M, I61L, | LEDGRTLSDYNLP | CCTTACTTTTCAAGGGAAGTTGCTGGAGGAC |
| | K63I, A44T, | ISPEKMAMLRM | GGTCGCACACTGTCTGACTATAACTTGCCTAT |
| | S49L, P38L, | Q | TTCTCCTGAGAAGATGGCCATGCTGCGCATG |
| | R74Q, M1Y, | | CAA |
| | V26I, L73M, | | |
| | P69A, L70M, | | |
| | L2M, L62P, | | |
| | D64S, K66E | | |
| CM433 | K6R, T7M, | YMIFVRMLTGK | TATATGATTTTCGTACGCATGTTGACTGGAAA |
| [611; 828] | T12M, T14E, | MIELEVEPSDTIE | GATGATCGAGTTGGAAGTGGAGCCTTCCGAT |
| | K33H, A46Q, | NIKAKIQDHEGIP | ACTATCGAGAATATTAAGGCCAAAATCCAAG |
| | S65P, L67K, | LDQQRLTFQGKL | ATCATGAAGGGATTCCTTTGGATCAACAACG |
| | H68M, I61L, | LEDGRTLSDYNLP | CCTTACTTTTCAAGGGAAGTTGCTGGAGGAC |
| | K63I, A44T, | ISPEKMAFLRMQ | GGTCGCACACTGTCTGACTATAACTTGCCTAT |
| | S49L, P38L, | | TTCTCCTGAGAAGATGGCCTTCCTGCGCATGC |
| | R74Q, M1Y, | | AA |
| | V26I, L73M, | | |
| | P69A, L70F, | | |
| | L2M, L62P, | | |
| | D64S, K66E | | |
| CM434 | K6R, T7M, | YMIFVRMLTGK | TATATGATTTTCGTACGCATGTTGACTGGAAA |
| [612; 829] | T12M, T14E, | MIELEVEPSDTIE | GATGATCGAGTTGGAAGTGGAGCCTTCCGAT |
| | K33H, A46Q, | NIKAKIQDHEGIP | ACTATCGAGAATATTAAGGCCAAAATCCAAG |
| | S65P, L67K, | LDQQRLTFQGKL | ATCATGAAGGGATTCCTTTGGATCAACAACG |
| | H68M, I61L, | LEDGRTLSDYNLP | CCTTACTTTTCAAGGGAAGTTGCTGGAGGAC |
| | K63I, A44T, | ISPEKMACLRMQ | GGTCGCACACTGTCTGACTATAACTTGCCTAT |
| | S49L, P38L, | | TTCTCCTGAGAAGATGGCCTGCCTGCGCATGC |
| | R74Q, M1Y, | | AA |
| | V26I, L73M, | | |
| | P69A, L70C, | | |
| | L2M, L62P, | | |
| | D64S, K66E | | |
| CM435 | K6R, T7M, | YMIFVRMLTGK | TATATGATTTTCGTACGCATGTTGACTGGAAA |
| [613; 830] | T12M, T14E, | MIELEVEPSDTIE | GATGATCGAGTTGGAAGTGGAGCCTTCCGAT |
| | K33H, A46Q, | NIKAKIQDHEGIP | ACTATCGAGAATATTAAGGCCAAAATCCAAG |
| | S65P, L67K, | LDQQRLTFQGKL | ATCATGAAGGGATTCCTTTGGATCAACAACG |
| | H68M, I61L, | LEDGRTLSDYNLP | CCTTACTTTTLAAGGGAAGTTGCTGGAGGAC |
| | K63I, A44T, | ISPEKMGMLRM | GGTCGCACACTGTCTGACTATAACTTGCCTAT |
| | S49L, P38L, | Q | TTCTCCTGAGAAGATGGGGATGCTGCGCATG |
| | R74Q, M1Y, | | CAA |
| | V26I, L73M, | | |
| | P69G, L70M, | | |
| | L2M, L62P, | | |
| | D64S, K66E | | |
| CM436 | K6R, T7M, | YMIFVRMLTGK | TATATGATTTTCGTACGCATGTTGACTGGAAA |
| [614; 831] | T12M, T14E, | MIELEVEPSDTIE | GATGATCGAGTTGGAAGTGGAGCCTTCCGAT |
| | K33H, A46Q, | NIKAKIQDHEGIP | ACTATCGAGAATATTAAGGCCAAAATCCAAG |
| | S65P, L67K, | LDQQRLTFQGKL | ATCATGAAGGGATTCCTTTGGATCAACAACG |
| | H68M, I61L, | LEDGRTLSDYNLP | CCTTACTTTTCAAGGGAAGTTGCTGGAGGAC |
| | K63I, A44T, | ISPEKMGFLRMQ | GGTCGCACACTGTCTGACTATAACTTGCCTAT |
| | S49L, P38L, | | TTCTCCTGAGAAGATGGGGTTCCTGCGCATG |
| | R74Q, M1Y, | | CAA |
| | V26I, L73M, | | |
| | P69G, L70F, | | |
| | L2M, L62P, | | |
| | D64S, K66E | | |
| CM437 | K6R, T7M, | YMIFVRMLTGK | TATATGATTTTCGTACGCATGTTGACTGGAAA |
| [615; 832] | T12M, T14E, | MIELEVEPSDTIE | GATGATCGAGTTGGAAGTGGAGCCTTCCGAT |
| | K33H, A46Q, | NIKAKIQDHEGIP | ACTATCGAGAATATTAAGGCCAAAATCCAAG |
| | S65P, L67K, | LDQQRLTFQGKL | ATCATGAAGGGATTCCTTTGGATCAACAACG |
| | H68M, I61L, | LEDGRTLSDYNLP | CCTTACTTTTCAAGGGAAGTTGCTGGAGGAC |
| | K63I, A44T, | ISPEKMGCLRMQ | GGTCGCACACTGTCTGACTATAACTTGCCTAT |
| | S49L, P38L, | | TTCTCCTGAGAAGATGGGGTGCCTGCGCATG |
| | R74Q, M1Y, | | CAA |
| | V26I, L73M, | | |
| | P69G, L70C, | | |
| | L2M, L62P, | | |
| | D64S, K66E | | |
| CM438 | K6R, T7M, | YMIFVRMLTGK | TATATGATTTTCGTACGCATGTTGACTGGAAA |
| [616; 833] | T12M, T14E, | MIELEVEPSDTIE | GATGATCGAGTTGGAAGTGGAGCCTTCCGAT |
| | K33H, A46Q, | NIKAKIQDHEGIP | ACTATCGAGAATATTAAGGCCAAAATCCAAG |
| | S65P, L67K, | LDQQRLTFQGKL | ATCATGAAGGGATTCCTTTGGATCAACAACG |
| | H68M, I61L, | LEDGRTLSDYNLP | CCTTACTTTTCAAGGGAAGTTGCTGGAGGAC |
| | K63I, A44T, | ISPEKMCMLRM | GGTCGCACACTGTCTGACTATAACTTGCCTAT |
| | S49L, P38L, | Q | TTCTCCTGAGAAGATGTGCATGCTGCGCATGC |
| | R74Q, M1Y, | | AA |
| | V26I, L73M, | | |
| | P69C, L70M, | | |
| | L2M, L62P, | | |
| | D64S, K66E | | |
| CM439 | K6R, T7M, | YMIFVRMLTGK | TATATGATTTTCGTACGCATGTTGACTGGAAA |
| [617; 834] | T12M, T14E, | MIELEVEPSDTIE | GATGATCGAGTTGGAAGTGGAGCCTTCCGAT |
| | K33H, A46Q, | NIKAKIQDHEGIP | ACTATCGAGAATATTAAGGCCAAAATCCAAG |
| | S65P, L67K, | LDQQRLTFQGKL | ATCATGAAGGGATTCCTTTGGATCAACAACG |
| | H68M, I61L, | LEDGRTLSDYNLP | CCTTACTTTTCAAGGGAAGTTGCTGGAGGAC |
| | K63I, A44T, | ISPEKMMMLRM | GGTCGCACACTGTCTGACTATAACTTGCCTAT |
| | S49L, P38L, | Q | TTCTCCTGAGAAGATGATGATGCTGCGCATG |
| | R74Q, M1Y, | | CAA |
| | V26I, L73M, | | |
| | P69M, L70M, | | |
| | L2M, L62P, | | |
| | D64S, K66E | | |
| CM440 | K6R, T7M, | YMIFVRMLTGK | TATATGATTTTCGTACGCATGTTGACTGGAAA |
| [618; 835] | T12M, T14E, | MIELEVEPSDTIE | GATGATCGAGTTGGAAGTGGAGCCTTCCGAT |
| | K33H, A46Q, | NIKAKIQDHEGIP | ACTATCGAGAATATTAAGGCCAAAATCCAAG |
| | S65P, L67K, | LDQQRLTFQGKL | ATCATGAAGGGATTCCTTTGGATCAACAACG |
| | H68M, I61L, | LEDGRTLSDYNLP | CCTTACTTTTCAAGGGAAGTTGCTGGAGGAC |
| | K63I, A44T, | ISPEKMFMLRM | GGTCGCACACTGTCTGACTATAACTTGCCTAT |
| | S49L, P38L, | Q | TTCTCCTGAGAAGATGTTCATGCTGCGCATGC |
| | R74Q, M1Y, | | AA |
| | V26I, L73M, | | |
| | P69F, L70M, | | |
| | L2M, L62P, | | |
| | D64S, K66E | | |
| CM441 | K6R, T7M, | YMIFVRMLTGK | TATATGATTTTCGTACGCATGTTGACTGGAAA |
| [619; 836] | T12M, T14E, | MIELEVEPSDTIE | GATGATCGAGTTGGAAGTGGAGCCTTCCGAT |
| | K33H, A46Q, | NIKAKIQDHEGIP | ACTATCGAGAATATTAAGGCCAAAATCCAAG |
| | S65P, L67K, | LDQQRLTFQGKL | ATCATGAAGGGATTCCTTTGGATCAACAACG |
| | H68M, I61L, | LEDGRTLSDYNL | CCTTACTTTTCAAGGGAAGTTGCTGGAGGAC |
| | K63I, A44T, | AISPEKMAVLRM | GGTCGCACACTGTCTGACTATAACTTGGCCAT |
| | S49L, P38L, | Q | TTCTCCTGAGAAGATGGCCGTACTGCGCATG |
| | R74Q, M1Y, | | CAA |
| | V26I, L73M, | | |
| | P69A, L70V, | | |
| | L2M, L62A, | | |
| | D64S, K66E | | |
| CM442 | K6R, T7M, | YMIFVRMLTGK | TATATGATTTTCGTACGCATGTTGACTGGAAA |
| [620; 837] | T12M, T14E, | MIELEVEPSDTIE | GATGATCGAGTTGGAAGTGGAGCCTTCCGAT |
| | K33H, A46Q, | NIKAKIQDHEGIP | ACTATCGAGAATATTAAGGCCAAAATCCAAG |
| | S65P, L67K, | LDQQRLTFQGKL | ATCATGAAGGGATTCCTTTGGATCAACAACG |
| | H68M, I61L, | LEDGRTLSDYNLR | CCTTACTTTTLAAGGGAAGTTGCTGGAGGAC |
| | K63I, A44T, | ISPEKMAVLRMQ | GGTCGCACACTGTCTGACTATAACTTGCGCAT |
| | S49L, P38L, | | TTCTCCTGAGAAGATGGCCGTACTGCGCATG |
| | R74Q, M1Y, | | CAA |
| | V26I, L73M, | | |
| | P69A, L70V, | | |
| | L2M, L62R, | | |
| | D64S, K66E | | |
| CM443 | K6R, T7M, | YMIFVRMLTGK | TATATGATTTTCGTACGCATGTTGACTGGAAA |
| [621; 838] | T12M, T14E, | MIELEVEPSDTIE | GATGATCGAGTTGGAAGTGGAGCCTTCCGAT |
| | K33H, A46Q, | NIKAKIQDHEGIP | ACTATCGAGAATATTAAGGCCAAAATCCAAG |
| | S65P, L67K, | LDQQRLTFQGKL | ATCATGAAGGGATTCCTTTGGATCAACAACG |
| | H68M, I61L, | LEDGRTLSDYNL | CCTTACTTTTLAAGGGAAGTTGCTGGAGGAC |
| | K63I, A44T, | NISPEKMAVLRM | GGTCGCACACTGTCTGACTATAACTTGAATAT |
| | S49L, P38L, | Q | TTCTCCTGAGAAGATGGCCGTACTGCGCATG |
| | R74Q, M1Y, | | CAA |
| | V26I, L73M, | | |
| | P69A, L70V, | | |
| | L2M, L62N, | | |
| | D64S, K66E | | |
| CM444 | K6R, T7M, | YMIFVRMLTGK | TATATGATTTTCGTACGCATGTTGACTGGAAA |
| [622; 839] | T12M, T14E, | MIELEVEPSDTIE | GATGATCGAGTTGGAAGTGGAGCCTTCCGAT |
| | K33H, A46Q, | NIKAKIQDHEGIP | ACTATCGAGAATATTAAGGCCAAAATCCAAG |
| | S65P, L67K, | LDQQRLTFQGKL | ATCATGAAGGGATTCCTTTGGATCAACAACG |
| | H68M, I61L, | LEDGRTLSDYNL | CCTTACTTTTLAAGGGAAGTTGCTGGAGGAC |
| | K63I, A44T, | DISPEKMAVLRM | GGTCGCACACTGTCTGACTATAACTTGGATAT |
| | S49L, P38L, | Q | TTCTCCTGAGAAGATGGCCGTACTGCGCATG |
| | R74Q, M1Y, | | CAA |
| | V26I, L73M, | | |
| | P69A, L70V, | | |
| | L2M, L62D, | | |
| | D64S, K66E | | |
| CM445 | K6R, T7M, | YMIFVRMLTGK | TATATGATTTTCGTACGCATGTTGACTGGAAA |
| [623; 840] | T12M, T14E, | MIELEVEPSDTIE | GATGATCGAGTTGGAAGTGGAGCCTTCCGAT |
| | K33H, A46Q, | NIKAKIQDHEGIP | ACTATCGAGAATATTAAGGCCAAAATCCAAG |
| | S65P, L67K, | LDQQRLTFQGKL | ATCATGAAGGGATTCCTTTGGATCAACAACG |
| | H68M, I61L, | LEDGRTLSDYNLC | CCTTACTTTTLAAGGGAAGTTGCTGGAGGAC |
| | K63I, A44T, | ISPEKMAVLRMQ | GGTCGCACACTGTCTGACTATAACTTGTGCAT |
| | S49L, P38L, | | TTCTCCTGAGAAGATGGCCGTACTGCGCATG |
| | R74Q, M1Y, | | CAA |
| | V26I, L73M, | | |
| | P69A, L70V, | | |
| | L2M, L62C, | | |
| | D64S, K66E | | |
| CM446 | K6R, T7M, | YMIFVRMLTGK | TATATGATTTTCGTACGCATGTTGACTGGAAA |
| [624; 841] | T12M, T14E, | MIELEVEPSDTIE | GATGATCGAGTTGGAAGTGGAGCCTTCCGAT |
| | K33H, A46Q, | NIKAKIQDHEGIP | ACTATCGAGAATATTAAGGCCAAAATCCAAG |
| | S65P, L67K, | LDQQRLTFQGKL | ATCATGAAGGGATTCCTTTGGATCAACAACG |
| | H68M, I61L, | LEDGRTLSDYNLE | CCTTACTTTTLAAGGGAAGTTGCTGGAGGAC |
| | K63I, A44T, | ISPEKMAVLRMQ | GGTCGCACACTGTCTGACTATAACTTGGAGAT |
| | S49L, P38L, | | TTCTCCTGAGAAGATGGCCGTACTGCGCATG |
| | R74Q, M1Y, | | CAA |
| | V26I, L73M, | | |
| | P69A, L70V, | | |
| | L2M, L62E, | | |
| | D64S, K66E | | |
| CM447 | K6R, T7M, | YMIFVRMLTGK | TATATGATTTTCGTACGCATGTTGACTGGAAA |
| [625; 842] | T12M, T14E, | MIELEVEPSDTIE | GATGATCGAGTTGGAAGTGGAGCCTTCCGAT |
| | K33H, A46Q, | NIKAKIQDHEGIP | ACTATCGAGAATATTAAGGCCAAAATCCAAG |
| | S65P, L67K, | LDQQRLTFQGKL | ATCATGAAGGGATTCCTTTGGATCAACAACG |
| | H68M, I61L, | LEDGRTLSDYNL | CLTTACTTTTCAAGGGAAGTTGCTGGAGGAC |
| | K63I, A44T, | QISPEKMAVLRM | GGTCGCACACTGTCTGACTATAACTTGCAAAT |
| | S49L, P38L, | Q | TTCTCCTGAGAAGATGGCCGTACTGCGCATG |
| | R74Q, M1Y, | | CAA |
| | V26I, L73M, | | |
| | P69A, L70V, | | |
| | L2M, L620, | | |
| | D64S, K66E | | |
| CM448 | K6R, T7M, | YMIFVRMLTGK | TATATGATTTTCGTACGCATGTTGACTGGAAA |
| [626; 843] | T12M, T14E, | MIELEVEPSDTIE | GATGATCGAGTTGGAAGTGGAGCCTTCCGAT |
| | K33H, A46Q, | NIKAKIQDHEGIP | ACTATCGAGAATATTAAGGCCAAAATCCAAG |
| | S65P, L67K, | LDQQRLTFQGKL | ATCATGAAGGGATTCCTTTGGATCAACAACG |
| | H68M, I61L, | LEDGRTLSDYNL | CCTTACTTTTCAAGGGAAGTTGCTGGAGGAC |
| | K63I, A44T, | GISPEKMAVLRM | GGTCGCACACTGTCTGACTATAACTTGGGGA |
| | S49L, P38L, | Q | TTTCTCCTGAGAAGATGGCCGTACTGCGCATG |
| | R74Q, M1Y, | | CAA |
| | V26I, L73M, | | |
| | P69A, L70V, | | |
| | L2M, L62G, | | |
| | D64S, K66E | | |
| CM449 | K6R, T7M, | YMIFVRMLTGK | TATATGATTTTCGTACGCATGTTGACTGGAAA |
| [627; 844] | T12M, T14E, | MIELEVEPSDTIE | GATGATCGAGTTGGAAGTGGAGCCTTCCGAT |
| | K33H, A46Q, | NIKAKIQDHEGIP | ACTATCGAGAATATTAAGGCCAAAATCCAAG |
| | S65P, L67K, | LDQQRLTFQGKL | ATCATGAAGGGATTCCTTTGGATCAACAACG |
| | H68M, I61L, | LEDGRTLSDYNL | CCTTACTTTTCAAGGGAAGTTGCTGGAGGAC |
| | K63I, A44T, | HISPEKMAVLRM | GGTCGCACACTGTCTGACTATAACTTGCATAT |
| | S49L, P38L, | Q | TTCTCCTGAGAAGATGGCCGTACTGCGCATG |
| | R74Q, M1Y, | | CAA |
| | V26I, L73M, | | |
| | P69A, L70V, | | |
| | L2M, L62H, | | |
| | D64S, K66E | | |
| CM450 | K6R, T7M, | YMIFVRMLTGK | TATATGATTTTCGTACGCATGTTGACTGGAAA |
| [628; 845] | T12M, T14E, | MIELEVEPSDTIE | GATGATCGAGTTGGAAGTGGAGCCTTCCGAT |
| | K33H, A46Q, | NIKAKIQDHEGIP | ACTATCGAGAATATTAAGGCCAAAATCCAAG |
| | S65P, L67K, | LDQQRLTFQGKL | ATCATGAAGGGATTCCTTTGGATCAACAACG |
| | H68M, I61L, | LEDGRTLSDYNLII | CCTTACTTTTCAAGGGAAGTTGCTGGAGGAC |
| | K63I, A44T, | SPEKMAVLRMQ | GGTCGCACACTGTCTGACTATAACTTGATTAT |
| | S49L, P38L, | | TTCTCCTGAGAAGATGGCCGTACTGCGCATG |
| | R74Q, M1Y, | | CAA |
| | V26I, L73M, | | |
| | P69A, L70V, | | |
| | L2M, L62I, | | |
| | D64S, K66E | | |
| CM451 | K6R, T7M, | YMIFVRMLTGK | TATATGATTTTCGTACGCATGTTGACTGGAAA |
| [629; 846] | T12M, T14E, | MIELEVEPSDTIE | GATGATCGAGTTGGAAGTGGAGCCTTCCGAT |
| | K33H, A46Q, | NIKAKIQDHEGIP | ACTATCGAGAATATTAAGGCCAAAATCCAAG |
| | S65P, L67K, | LDQQRLTFQGKL | ATCATGAAGGGATTCCTTTGGATCAACAACG |
| | H68M, I61L, | LEDGRTLSDYNLK | CCTTACTTTTCAAGGGAAGTTGCTGGAGGAC |
| | K63I, A44T, | ISPEKMAVLRMQ | GGTCGCACACTGTCTGACTATAACTTGAAGAT |
| | S49L, P38L, | | TTCTCCTGAGAAGATGGCCGTACTGCGCATG |
| | R74Q, M1Y, | | CAA |
| | V26I, L73M, | | |
| | P69A, L70V, | | |
| | L2M, L62K, | | |
| | D64S, K66E | | |
| CM452 | K6R, T7M, | YMIFVRMLTGK | TATATGATTTTCGTACGCATGTTGACTGGAAA |
| [630; 847] | T12M, T14E, | MIELEVEPSDTIE | GATGATCGAGTTGGAAGTGGAGCCTTCCGAT |
| | K33H, A46Q, | NIKAKIQDHEGIP | ACTATCGAGAATATTAAGGCCAAAATCCAAG |
| | S65P, L67K, | LDQQRLTFQGKL | ATCATGAAGGGATTCCTTTGGATCAACAACG |
| | H68M, I61L, | LEDGRTLSDYNL | CCTTACTTTTLAAGGGAAGTTGCTGGAGGAC |
| | K63I, A44T, | MISPEKMAVLR | GGTCGCACACTGTCTGACTATAACTTGATGAT |
| | S49L, P38L, | MO | TTCTCCTGAGAAGATGGCCGTACTGCGCATG |
| | R74Q, M1Y, | | CAA |
| | V26I, L73M, | | |
| | P69A, L70V, | | |
| | L2M, L62M, | | |
| | D64S, K66E | | |
| CM453 | K6R, T7M, | YMIFVRMLTGK | TATATGATTTTCGTACGCATGTTGACTGGAAA |
| [631; 848] | T12M, T14E, | MIELEVEPSDTIE | GATGATCGAGTTGGAAGTGGAGCCTTCCGAT |
| | K33H, A46Q, | NIKAKIQDHEGIP | ACTATCGAGAATATTAAGGCCAAAATCCAAG |
| | S65P, L67K, | LDQQRLTFQGKL | ATCATGAAGGGATTCCTTTGGATCAACAACG |
| | H68M, I61L, | LEDGRTLSDYNLF | CCTTACTTTTLAAGGGAAGTTGCTGGAGGAC |
| | K63I, A44T, | ISPEKMAVLRMQ | GGTCGCACACTGTCTGACTATAACTTGTTCAT |
| | S49L, P38L, | | TTCTCCTGAGAAGATGGCCGTACTGCGCATG |
| | R74Q, M1Y, | | CAA |
| | V26I, L73M, | | |
| | P69A, L70V, | | |
| | L2M, L62F, | | |
| | D64S, K66E | | |
| CM454 | K6R, T7M, | YMIFVRMLTGK | TATATGATTTTCGTACGCATGTTGACTGGAAA |
| [632; 849] | T12M, T14E, | MIELEVEPSDTIE | GATGATCGAGTTGGAAGTGGAGCCTTCCGAT |
| | K33H, A46Q, | NIKAKIQDHEGIP | ACTATCGAGAATATTAAGGCCAAAATCCAAG |
| | S65P, L67K, | LDQQRLTFQGKL | ATCATGAAGGGATTCCTTTGGATCAACAACG |
| | H68M, I61L, | LEDGRTLSDYNLS | CCTTACTTTTLAAGGGAAGTTGCTGGAGGAC |
| | K63I, A44T, | ISPEKMAVLRMQ | GGTCGCACACTGTCTGACTATAACTTGTCTAT |
| | S49L, P38L, | | TTCTCCTGAGAAGATGGCCGTACTGCGCATG |
| | R74Q, M1Y, | | CAA |
| | V26I, L73M, | | |
| | P69A, L70V, | | |
| | L2M, L62S, | | |
| | D64S, K66E | | |
| CM455 | K6R, T7M, | YMIFVRMLTGK | TATATGATTTTCGTACGCATGTTGACTGGAAA |
| [633; 850] | T12M, T14E, | MIELEVEPSDTIE | GATGATCGAGTTGGAAGTGGAGCCTTCCGAT |
| | K33H, A46Q, | NIKAKIQDHEGIP | ACTATCGAGAATATTAAGGCCAAAATCCAAG |
| | S65P, L67K, | LDQQRLTFQGKL | ATCATGAAGGGATTCCTTTGGATCAACAACG |
| | H68M, I61L, | LEDGRTLSDYNLT | CCTTACTTTTLAAGGGAAGTTGCTGGAGGAC |
| | K63I, A44T, | ISPEKMAVLRMQ | GGTCGCACACTGTCTGACTATAACTTGACTAT |
| | S49L, P38L, | | TTCTCCTGAGAAGATGGCCGTACTGCGCATG |
| | R74Q, M1Y, | | CAA |
| | V26I, L73M, | | |
| | P69A, L70V, | | |
| | L2M, L62T, | | |
| | D64S, K66E | | |
| CM456 | K6R, T7M, | YMIFVRMLTGK | TATATGATTTTCGTACGCATGTTGACTGGAAA |
| [634; 851] | T12M, T14E, | MIELEVEPSDTIE | GATGATCGAGTTGGAAGTGGAGCCTTCCGAT |
| | K33H, A46Q, | NIKAKIQDHEGIP | ACTATCGAGAATATTAAGGCCAAAATCCAAG |
| | S65P, L67K, | LDQQRLTFQGKL | ATCATGAAGGGATTCCTTTGGATCAACAACG |
| | H68M, I61L, | LEDGRTLSDYNL | CCTTACTTTTCAAGGGAAGTTGCTGGAGGAC |
| | K63I, A44T, | WISPEKMAVLR | GGTCGCACACTGTCTGACTATAACTTGTGGAT |
| | S49L, P38L, | MQ | TTCTCCTGAGAAGATGGCCGTACTGCGCATG |
| | R74Q, M1Y, | | CAA |
| | V26I, L73M, | | |
| | P69A, L70V, | | |
| | L2M, L62W, | | |
| | D64S, K66E | | |
| CM457 | K6R, T7M, | YMIFVRMLTGK | TATATGATTTTCGTACGCATGTTGACTGGAAA |
| [635; 852] | T12M, T14E, | MIELEVEPSDTIE | GATGATCGAGTTGGAAGTGGAGCCTTCCGAT |
| | K33H, A46Q, | NIKAKIQDHEGIP | ACTATCGAGAATATTAAGGCCAAAATCCAAG |
| | S65P, L67K, | LDQQRLTFQGKL | ATCATGAAGGGATTCCTTTGGATCAACAACG |
| | H68M, I61L, | LEDGRTLSDYNLY | CCTTACTTTTLAAGGGAAGTTGCTGGAGGAC |
| | K63I, A44T, | ISPEKMAVLRMQ | GGTCGCACACTGTCTGACTATAACTTGTATAT |
| | S49L, P38L, | | TTCTCCTGAGAAGATGGCCGTACTGCGCATG |
| | R74Q, M1Y, | | CAA |
| | V26I, L73M, | | |
| | P69A, L70V, | | |
| | L2M, L62Y, | | |
| | D64S, K66E | | |
| CM458 | K6R, T7M, | YMIFVRMLTGK | TATATGATTTTCGTACGCATGTTGACTGGAAA |
| [636; 853] | T12M, T14E, | MIELEVEPSDTIE | GATGATCGAGTTGGAAGTGGAGCCTTCCGAT |
| | K33H, A46Q, | NIKAKIQDHEGIP | ACTATCGAGAATATTAAGGCCAAAATCCAAG |
| | S65P, L67K, | LDQQRLTFQGKL | ATCATGAAGGGATTCCTTTGGATCAACAACG |
| | H68M, I61L, | LEDGRTLSDYNL | CCTTACTTTTCAAGGGAAGTTGCTGGAGGAC |
| | K63I, A44T, | VISPEKMAVLRM | GGTCGCACACTGTCTGACTATAACTTGGTAAT |
| | S49L, P38L, | Q | TTCTCCTGAGAAGATGGCCGTACTGCGCATG |
| | R74Q, M1Y, | | CAA |
| | V26I, L73M, | | |
| | P69A, L70V, | | |
| | L2M, L62V, | | |
| | D64S, K66E | | |
| CM459 | K6R, T7M, | YMIFVRMLTGK | TATATGATTTTCGTACGCATGTTGACTGGAAA |
| [637; 854] | T12M, T14E, | MIELEVEPSDTIE | GATGATCGAGTTGGAAGTGGAGCCTTCCGAT |
| | K33H, A46Q, | NIKAKIQDHEGIP | ACTATCGAGAATATTAAGGCCAAAATCCAAG |
| | S65P, L67K, | LDQQRLTFQGKL | ATCATGAAGGGATTCCTTTGGATCAACAACG |
| | H68M, I61L, | LEDGRTLSDYNLP | CCTTACTTTTCAAGGGAAGTTGCTGGAGGAC |
| | K63I, A44T, | ISPEKMCFLRMQ | GGTCGCACACTGTCTGACTATAACTTGCCTAT |
| | S49L, P38L, | | TTCTCCTGAGAAGATGTGCTTCCTGCGCATGC |
| | R74Q, M1Y, | | AA |
| | V26I, L73M, | | |
| | P69C, L70F, | | |
| | L2M, L62P, | | |
| | D64S, K66E | | |
| CM460 | K6R, T7M, | YMIFVRMLTGK | TATATGATTTTCGTACGCATGTTGACTGGAAA |
| [638; 855] | T12M, T14E, | MIELEVEPSDTIE | GATGATCGAGTTGGAAGTGGAGCCTTCCGAT |
| | K33H, A46Q, | NIKAKIQDHEGIP | ACTATCGAGAATATTAAGGCCAAAATCCAAG |
| | S65P, L67K, | LDQQRLTFQGKL | ATCATGAAGGGATTCCTTTGGATCAACAACG |
| | H68M, I61L, | LEDGRTLSDYNLP | CCTTACTTTTCAAGGGAAGTTGCTGGAGGAC |
| | K63I, A44T, | ISPEKMMFLRM | GGTCGCACACTGTCTGACTATAACTTGCCTAT |
| | S49L, P38L, | Q | TTCTCCTGAGAAGATGATGTTCCTGCGCATGC |
| | R74Q, M1Y, | | AA |
| | V26I, L73M, | | |
| | P69M, L70F, | | |
| | L2M, L62P, | | |
| | D64S, K66E | | |
| CM461 | K6R, T7M, | YMIFVRMLTGK | TATATGATTTTCGTACGCATGTTGACTGGAAA |
| [639; 856] | T12M, T14E, | MIELEVEPSDTIE | GATGATCGAGTTGGAAGTGGAGCCTTCCGAT |
| | K33H, A46Q, | NIKAKIQDHEGIP | ACTATCGAGAATATTAAGGCCAAAATCCAAG |
| | S65P, L67K, | LDQQRLTFQGKL | ATCATGAAGGGATTCCTTTGGATCAACAACG |
| | H68M, I61L, | LEDGRTLSDYNLP | CCTTACTTTTLAAGGGAAGTTGCTGGAGGAC |
| | K63I, A44T, | ISPEKMFFLRMQ | GGTCGCACACTGTCTGACTATAACTTGCCTAT |
| | S49L, P38L, | | TTCTCCTGAGAAGATCilTCTTCCTGCGCATaC |
| | R74Q, M1Y, | | AA |
| | V26I, L73M, | | |
| | P69F, L70F, | | |
| | L2M, L62P, | | |
| | D64S, K66E | | |
| CM462 | K6R, T7M, | YMIFVRMLTGK | TATATGATTTTCGTACGCATGTTGACTGGAAA |
| [640; 857] | T12M, T14E, | MIELEVEPSDTIE | GATGATCGAGTTGGAAGTGGAGCCTTCCGAT |
| | K33H, A46Q, | NIKAKIQDHEGIP | ACTATCGAGAATATTAAGGCCAAAATCCAAG |
| | S65P, L67K, | LDQQRLTFQGKL | ATCATGAAGGGATTCCTTTGGATCAACAACG |
| | H68M, I61L, | LEDGRTLSDYNLP | CCTTACTTTTLAAGGGAAGTTGCTGGAGGAC |
| | K63I, A44T, | ISPEKMCCLRMQ | GGTCGCACACTGTCTGACTATAACTTGCCTAT |
| | S49L, P38L, | | TTCTCCTGAGAAGATGTGCTGCCTGCGCATGC |
| | R74Q, M1Y, | | AA |
| | V26I, L73M, | | |
| | P69C, L70C, | | |
| | L2M, L62P, | | |
| | D64S, K66E | | |
| CM463 | K6R, T7M, | YMIFVRMLTGK | TATATGATTTTCGTACGCATGTTGACTGGAAA |
| [641; 858] | T12M, T14E, | MIELEVEPSDTIE | GATGATCGAGTTGGAAGTGGAGCCTTCCGAT |
| | K33H, A46Q, | NIKAKIQDHEGIP | ACTATCGAGAATATTAAGGCCAAAATCCAAG |
| | S65P, L67K, | LDQQRLTFQGKL | ATCATGAAGGGATTCCTTTGGATCAACAACG |
| | H68M, I61L, | LEDGRTLSDYNLP | CCTTACTTTTLAAGGGAAGTTGCTGGAGGAC |
| | K63I, A44T, | ISPEKMMCLRM | GGTCGCACACTGTCTGACTATAACTTGCCTAT |
| | S49L, P38L, | Q | TTCTCCTGAGAAGATGATGTGCCTGCGCATGC |
| | R74Q, M1Y, | | AA |
| | V26I, L73M, | | |
| | P69M, L70C, | | |
| | L2M, L62P, | | |
| | D64S, K66E | | |
| CM464 | K6R, T7M, | YMIFVRMLTGK | TATATGATTTTCGTACGCATGTTGACTGGAAA |
| [642; 859] | T12M, T14E, | MIELEVEPSDTIE | GATGATCGAGTTGGAAGTGGAGCCTTCCGAT |
| | K33H, A46Q, | NIKAKIQDHEGIP | ACTATCGAGAATATTAAGGCCAAAATCCAAG |
| | S65P, L67K, | LDQQRLTFQGKL | ATCATGAAGGGATTCCTTTGGATCAACAACG |
| | H68N, I61L, | LEDGRTLSDYNLP | CCTTACTTTTLAAGGGAAGTTGCTGGAGGAC |
| | K63I, A44T, | ISPEKMFCLRMQ | GGTCGCACACTGTCTGACTATAACTTGCCTAT |
| | S49L, P38L, | | TTCTCCTGAGAAGATGTTCTGCCTGCGCATGC |
| | R74Q, M1Y, | | AA |
| | V26I, L73M, | | |
| | P69F, L70C, | | |
| | L2M, L62P, | | |
| | D64S, K66E | | |
| CM465 | K6R, T7M, | YMIFVRMLTGK | TATATGATTTTCGTACGCATGTTGACTGGAAA |
| [643; 860] | T12M, T14E, | MIELEVEPSDTIE | GATGATCGAGTTGGAAGTGGAGCCTTCCGAT |
| | K33H, A46Q, | NIKAKIQDHEGIP | ACTATCGAGAATATTAAGGCCAAAATCCAAG |
| | S65P, L67K, | LDQQRLTFQGKL | ATCATGAAGGGATTCCTTTGGATCAACAACG |
| | H68M, I61L, | LEDGRTLSDYNL | CCTTACTTTTCAAGGGAAGTTGCTGGAGGAC |
| | K63I, A44T, | AISPEKMAMLR | GGTCGCACACTGTCTGACTATAACTTGGCCAT |
| | S49L, P38L, | MQ | TTCTCCTGAGAAGATGGCCATGCTGCGCATG |
| | R74Q, M1Y, | | CAA |
| | V26I, L73M, | | |
| | P69A, L70M, | | |
| | L2M, L62A, | | |
| | D64S, K66E | | |
| CM467 | K6R, T7M, | YMIFVRMLTGK | TATATGATTTTCGTACGCATGTTGACTGGAAA |
| [644; 861] | T12M, T14E, | MIELEVEPSDTIE | GATGATCGAGTTGGAAGTGGAGCCTTCCGAT |
| | K33H, A46Q, | NIKAKIQDHEGIP | ACTATCGAGAATATTAAGGCCAAAATCCAAG |
| | S65P, L67K, | LDQQRLTFQGKL | ATCATGAAGGGATTCCTTTGGATCAACAACG |
| | H68M, I61L, | LEDGRTLSDYNLC | CLTTACTTTTCAAGGGAAGTTGCTGGAGGAC |
| | K63I, A44T, | ISPEKMAMLRM | GGTCGCACACTGTCTGACTATAACTTGTGCAT |
| | S49L, P38L, | Q | TTCTCCTGAGAAGATGGCCATGCTGCGCATG |
| | R74Q, M1Y, | | CAA |
| | V26I, L73M, | | |
| | P69A, L70M, | | |
| | L2M, L62C, | | |
| | D64S, K66E | | |
| CM468 | K6R, T7M, | YMIFVRMLTGK | TATATGATTTTCGTACGCATGTTGACTGGAAA |
| [645; 862] | T12M, T14E, | MIELEVEPSDTIE | GATGATCGAGTTGGAAGTGGAGCCTTCCGAT |
| | K33H, A46Q, | NIKAKIQDHEGIP | ACTATCGAGAATATTAAGGCCAAAATCCAAG |
| | S65P, L67K, | LDQQRLTFQGKL | ATCATGAAGGGATTCCTTTGGATCAACAACG |
| | H68M, I61L, | LEDGRTLSDYNLT | CCTTACTTTTCAAGGGAAGTTGCTGGAGGAC |
| | K63I, A44T, | ISPEKMAMLRM | GGTCGCACACTGTCTGACTATAACTTGACTAT |
| | S49L, P38L, | Q | TTCTCCTGAGAAGATGGCCATGCTGCGCATG |
| | R74Q, M1Y, | | CAA |
| | V26I, L73M, | | |
| | P69A, L70M, | | |
| | L2M, L62T, | | |
| | D64S, K66E | | |
| CM469 | K6R, T7M, | YMIFVRMLTGK | TATATGATTTTCGTACGCATGTTGACTGGAAA |
| [646; 863] | T12M, T14E, | MIELEVEPSDTIE | GATGATCGAGTTGGAAGTGGAGCCTTCCGAT |
| | K33H, A46Q, | NIKAKIQDHEGIP | ACTATCGAGAATATTAAGGCCAAAATCCAAG |
| | S65P, L67K, | LDQQRLTFQGKL | ATCATGAAGGGATTCCTTTGGATCAACAACG |
| | H68M, I61L, | LEDGRTLSDYNL | CCTTACTTTTCAAGGGAAGTTGCTGGAGGAC |
| | K63I, A44T, | VISPEKMAMLR | GGTCGCACACTGTCTGACTATAACTTGGTAAT |
| | S49L, P38L, | MQ | TTCTCCTGAGAAGATGGCCATGCTGCGCATG |
| | R74Q, M1Y, | | CAA |
| | V26I, L73M, | | |
| | P69A, L70M, | | |
| | L2M, L62V, | | |
| | D64S, K66E | | |
| CM478 | K6R, T7M, | YAIFVRMLTGKM | TATGCCATTTTCGTACGCATGTTGACTGGAAA |
| [647; 864] | T12M, T14E, | IELEVEPSDTIENI | GATGATCGAGTTGGAAGTGGAGCCTTCCGAT |
| | K33H, A46Q, | KAKIQDHEGIPLD | ACTATCGAGAATATTAAGGCCAAAATCCAAG |
| | S65P, L67K, | QQRLTFQGKLLE | ATCATGAAGGGATTCCTTTGGATCAACAACG |
| | H68M, I61L, | DGRTLSDYNLTIS | CLTTACTTTTCAAGGGAAGTTGCTGGAGGAC |
| | K63I, A44T, | PEKMAVLRMQ | GGTCGCACACTGTCTGACTATAACTTGACTAT |
| | S49L, P38L, | | TTCTCCTGAGAAGATGGCCGTACTGCGCATG |
| | R74Q, M1Y, | | CAA |
| | V26I, L73M, | | |
| | P69A, L70V, | | |
| | L2A, L62T, | | |
| | D64S, K66E | | |
| CM479 | K6R, T7M, | YRIFVRMLTGKM | TATCGCATTTTCGTACGCATGTTGACTGGAAA |
| [648; 865] | T12M, T14E, | IELEVEPSDTIENI | GATGATCGAGTTGGAAGTGGAGCCTTCCGAT |
| | K33H, A46Q, | KAKIQDHEGIPLD | ACTATCGAGAATATTAAGGCCAAAATCCAAG |
| | S65P, L67K, | QQRLTFQGKLLE | ATCATGAAGGGATTCCTTTGGATCAACAACG |
| | H68M, I61L, | DGRTLSDYNLTIS | CCTTACTTTTCAAGGGAAGTTGCTGGAGGAC |
| | K63I, A44T, | PEKMAVLRMQ | GGTCGCACACTGTCTGACTATAACTTGACTAT |
| | S49L, P38L, | | TTCTCCTGAGAAGATGGCCGTACTGCGCATG |
| | R74Q, M1Y, | | CAA |
| | V26I, L73M, | | |
| | P69A, L70V, | | |
| | L2R, L62T, | | |
| | D64S, K66E | | |
| CM480 | K6R, T7M, | YNIFVRMLTGKM | TATAATATTTTCGTACGCATGTTGACTGGAAA |
| [649; 866] | T12M, T14E, | IELEVEPSDTIENI | GATGATCGAGTTGGAAGTGGAGCCTTCCGAT |
| | K33H, A46Q, | KAKIQDHEGIPLD | ACTATCGAGAATATTAAGGCCAAAATCCAAG |
| | S65P, L67K, | QQRLTFQGKLLE | ATCATGAAGGGATTCCTTTGGATCAACAACG |
| | H68M, I61L, | DGRTLSDYNLTIS | CCTTACTTTTCAAGGGAAGTTGCTGGAGGAC |
| | K63I, A44T, | PEKMAVLRMQ | GGTCGCACACTGTCTGACTATAACTTGACTAT |
| | S49L, P38L, | | TTCTCCTGAGAAGATGGCCGTACTGCGCATG |
| | R74Q, M1Y, | | CAA |
| | V26I, L73M, | | |
| | P69A, L70V, | | |
| | L2N, L62T, | | |
| | D64S, K66E | | |
| CM481 | K6R, T7M, | YDIFVRMLTGKM | TATGATATTTTCGTACGCATGTTGACTGGAAA |
| [650; 867] | T12M, T14E, | IELEVEPSDTIENI | GATGATCGAGTTGGAAGTGGAGCCTTCCGAT |
| | K33H, A46Q, | KAKIQDHEGIPLD | ACTATCGAGAATATTAAGGCCAAAATCCAAG |
| | S65P, L67K, | QQRLTFQGKLLE | ATCATGAAGGGATTCCTTTGGATCAACAACG |
| | H68M, I61L, | DGRTLSDYNLTIS | CCTTACTTTTLAAGGGAAGTTGCTGGAGGAC |
| | K63I, A44T, | PEKMAVLRMQ | GGTCGCACACTGTCTGACTATAACTTGACTAT |
| | S49L, P38L, | | TTCTCCTGAGAAGATGGCCGTACTGCGCATG |
| | R74Q, M1Y, | | CAA |
| | V26I, L73M, | | |
| | P69A, L70V, | | |
| | L2D, L62T, | | |
| | D64S, K66E | | |
| CM482 | K6R, T7M, | YCIFVRMLTGKM | TATTGCATTTTCGTACGCATGTTGACTGGAAA |
| [651; 868] | T12M, T14E, | IELEVEPSDTIENI | GATGATCGAGTTGGAAGTGGAGCCTTCCGAT |
| | K33H, A46Q, | KAKIQDHEGIPLD | ACTATCGAGAATATTAAGGCCAAAATCCAAG |
| | S65P, L67K, | QQRLTFQGKLLE | ATCATGAAGGGATTCCTTTGGATCAACAACG |
| | H68M, I61L, | DGRTLSDYNLTIS | CCTTACTTTTLAAGGGAAGTTGCTGGAGGAC |
| | K63I, A44T, | PEKMAVLRMQ | GGTCGCACACTGTCTGACTATAACTTGACTAT |
| | S49L, P38L, | | TTCTCCTGAGAAGATGGCCGTACTGCGCATG |
| | R74Q, M1Y, | | CAA |
| | V26I, L73M, | | |
| | P69A, L70V, | | |
| | L2C, L62T, | | |
| | D64S, K66E | | |
| CM483 | K6R, T7M, | YEIFVRMLTGKM | TATGAGATTTTCGTACGCATGTTGACTGGAAA |
| [652; 869] | T12M, T14E, | IELEVEPSDTIENI | GATGATCGAGTTGGAAGTGGAGCCTTCCGAT |
| | K33H, A46Q, | KAKIQDHEGIPLD | ACTATCGAGAATATTAAGGCCAAAATCCAAG |
| | S65P, L67K, | QQRLTFQGKLLE | ATCATGAAGGGATTCCTTTGGATCAACAACG |
| | H68M, I61L, | DGRTLSDYNLTIS | CCTTACTTTTCAAGGGAAGTTGCTGGAGGAC |
| | K63I, A44T, | PEKMAVLRMQ | GGTCGCACACTGTCTGACTATAACTTGACTAT |
| | S49L, P38L, | | TTCTCCTGAGAAGATGGCCGTACTGCGCATG |
| | R74Q, M1Y, | | CAA |
| | V26I, L73M, | | |
| | P69A, L70V, | | |
| | L2E, L62T, | | |
| | D64S, K66E | | |
| CM484 | K6R, T7M, | YQIFVRMLTGKM | TATCAAATTTTCGTACGCATGTTGACTGGAAA |
| [653; 870] | T12M, T14E, | IELEVEPSDTIENI | GATGATCGAGTTGGAAGTGGAGCCTTCCGAT |
| | K33H, A46Q, | KAKIQDHEGIPLD | ACTATCGAGAATATTAAGGCCAAAATCCAAG |
| | S65P, L67K, | QQRLTFQGKLLE | ATCATGAAGGGATTCCTTTGGATCAACAACG |
| | H68M, I61L, | DGRTLSDYNLTIS | CCTTACTTTTCAAGGGAAGTTGCTGGAGGAC |
| | K63I, A44T, | PEKMAVLRMQ | GGTCGCACACTGTCTGACTATAACTTGACTAT |
| | S49L, P38L, | | TTCTCCTGAGAAGATGGCCGTACTGCGCATG |
| | R74Q, M1Y, | | CAA |
| | V26I, L73M, | | |
| | P69A, L70V, | | |
| | L2Q, L62T, | | |
| | D64S, K66E | | |
| CM485 | K6R, T7M, | YGIFVRMLTGKM | TATGGGATTTTCGTACGCATGTTGACTGGAAA |
| [654; 871] | T12M, T14E, | IELEVEPSDTIENI | GATGATCGAGTTGGAAGTGGAGCCTTCCGAT |
| | K33H, A46Q, | KAKIQDHEGIPLD | ACTATCGAGAATATTAAGGCCAAAATCCAAG |
| | S65P, L67K, | QQRLTFQGKLLE | ATCATGAAGGGATTCCTTTGGATCAACAACG |
| | H68M, I61L, | DGRTLSDYNLTIS | CCTTACTTTTLAAGGGAAGTTGCTGGAGGAC |
| | K63I, A44T, | PEKMAVLRMQ | GGTCGCACACTGTCTGACTATAACTTGACTAT |
| | S49L, P38L, | | TTCTCCTGAGAAGATGGCCGTACTGCGCATG |
| | R74Q, M1Y, | | CAA |
| | V26I, L73M, | | |
| | P69A, L70V, | | |
| | L2G, L62T, | | |
| | D64S, K66E | | |
| CM486 | K6R, T7M, | YHIFVRMLTGKM | TATCATATTTTCGTACGCATGTTGACTGGAAA |
| [655; 872] | T12M, T14E, | IELEVEPSDTIENI | GATGATCGAGTTGGAAGTGGAGCCTTCCGAT |
| | K33H, A46Q, | KAKIQDHEGIPLD | ACTATCGAGAATATTAAGGCCAAAATCCAAG |
| | S65P, L67K, | QQRLTFQGKLLE | ATCATGAAGGGATTCCTTTGGATCAACAACG |
| | H68M, I61L, | DGRTLSDYNLTIS | CCTTACTTTTCAAGGGAAGTTGCTGGAGGAC |
| | K63I, A44T, | PEKMAVLRMQ | GGTCGCACACTGTCTGACTATAACTTGACTAT |
| | S49L, P38L, | | TTCTCCTGAGAAGATGGCCGTACTGCGCATG |
| | R74Q, M1Y, | | CAA |
| | V26I, L73M, | | |
| | P69A, L70V, | | |
| | L2H, L62T, | | |
| | D64S, K66E | | |
| CM487 | K6R, T7M, | YIIFVRMLTGKMI | TATATTATTTTCGTACGCATGTTGACTGGAAA |
| [656; 873] | T12M, T14E, | ELEVEPSDTIENIK | GATGATCGAGTTGGAAGTGGAGCCTTCCGAT |
| | K33H, A46Q, | AKIQDHEGIPLD | ACTATCGAGAATATTAAGGCCAAAATCCAAG |
| | S65P, L67K, | QQRLTFQGKLLE | ATCATGAAGGGATTCCTTTGGATCAACAACG |
| | H68M, I61L, | DGRTLSDYNLTIS | CCTTACTTTTCAAGGGAAGTTGCTGGAGGAC |
| | K63I, A44T, | PEKMAVLRMQ | GGTCGCACACTGTCTGACTATAACTTGACTAT |
| | S49L, P38L, | | TTCTCCTGAGAAGATGGCCGTACTGCGCATG |
| | R74Q, M1Y, | | CAA |
| | V26I, L73M, | | |
| | P69A, L70V, | | |
| | L2I, L62T, | | |
| | D64S, K66E | | |
| CM488 | K6R, T7M, | YKIFVRMLTGKM | TATAAGATTTTCGTACGCATGTTGACTGGAAA |
| [657; 874] | T12M, T14E, | IELEVEPSDTIENI | GATGATCGAGTTGGAAGTGGAGCCTTCCGAT |
| | K33H, A46Q, | KAKIQDHEGIPLD | ACTATCGAGAATATTAAGGCCAAAATCCAAG |
| | S65P, L67K, | QQRLTFQGKLLE | ATCATGAAGGGATTCCTTTGGATCAACAACG |
| | H68M, I61L, | DGRTLSDYNLTIS | CCTTACTTTTLAAGGGAAGTTGCTGGAGGAC |
| | K63I, A44T, | PEKMAVLRMQ | GGTCGCACACTGTCTGACTATAACTTGACTAT |
| | S49L, P38L, | | TTCTCCTGAGAAGATGGCCGTACTGCGCATG |
| | R74Q, M1Y, | | CAA |
| | V26I, L73M, | | |
| | P69A, L70V, | | |
| | L2K, L62T, | | |
| | D64S, K66E | | |
| CM489 | K6R, T7M, | YLIFVRMLTGKMI | TATTTGATTTTCGTACGCATGTTGACTGGAAA |
| [658; 875] | T12M, T14E, | ELEVEPSDTIENIK | GATGATCGAGTTGGAAGTGGAGCCTTCCGAT |
| | K33H, A46Q, | AKIQDHEGIPLD | ACTATCGAGAATATTAAGGCCAAAATCCAAG |
| | S65P, L67K, | QQRLTFQGKLLE | ATCATGAAGGGATTCCTTTGGATCAACAACG |
| | H68M, I61L, | DGRTLSDYNLTIS | CLTTACTTTTCAAGGGAAGTTGCTGGAGGAC |
| | K63I, A44T, | PEKMAVLRMQ | GGTCGCACACTGTCTGACTATAACTTGACTAT |
| | S49L, P38L, | | TTCTCCTGAGAAGATGGCCGTACTGCGCATG |
| | R74Q, M1Y, | | CAA |
| | V26I, L73M, | | |
| | P69A, L70V, | | |
| | L62T, D64S, | | |
| | K66E | | |
| CM490 | K6R, T7M, | YFIFVRMLTGKMI | TATTTCATTTTCGTACGCATGTTGACTGGAAA |
| [659; 876] | T12M, T14E, | ELEVEPSDTIENIK | GATGATCGAGTTGGAAGTGGAGCCTTCCGAT |
| | K33H, A46Q, | AKIQDHEGIPLD | ACTATCGAGAATATTAAGGCCAAAATCCAAG |
| | S65P, L67K, | QQRLTFQGKLLE | ATCATGAAGGGATTCCTTTGGATCAACAACG |
| | H68M, I61L, | DGRTLSDYNLTIS | CCTTACTTTTCAAGGGAAGTTGCTGGAGGAC |
| | K63I, A44T, | PEKMAVLRMQ | GGTCGCACACTGTCTGACTATAACTTGACTAT |
| | S49L, P38L, | | TTCTCCTGAGAAGATGGCCGTACTGCGCATG |
| | R74Q, M1Y, | | CAA |
| | V26I, L73M, | | |
| | P69A, L70V, | | |
| | L2F, L62T, | | |
| | D64S, K66E | | |
| CM491 | K6R, T7M, | YSIFVRMLTGKMI | TATTCTATTTTCGTACGCATGTTGACTGGAAA |
| [660; 877] | T12M, T14E, | ELEVEPSDTIENIK | GATGATCGAGTTGGAAGTGGAGCCTTCCGAT |
| | K33H, A46Q, | AKIQDHEGIPLD | ACTATCGAGAATATTAAGGCCAAAATCCAAG |
| | S65P, L67K, | QQRLTFQGKLLE | ATCATGAAGGGATTCCTTTGGATCAACAACG |
| | H68M, I61L, | DGRTLSDYNLTIS | CCTTACTTTTCAAGGGAAGTTGCTGGAGGAC |
| | K63I, A44T, | PEKMAVLRMQ | GGTCGCACACTGTCTGACTATAACTTGACTAT |
| | S49L, P38L, | | TTCTCCTGAGAAGATGGCCGTACTGCGCATG |
| | R74Q, M1Y, | | CAA |
| | V26I, L73M, | | |
| | P69A, L70V, | | |
| | L2S, L62T, | | |
| | D64S, K66E | | |
| CM492 | K6R, T7M, | YTIFVRMLTGKM | TATACTATTTTCGTACGCATGTTGACTGGAAA |
| [661; 878] | T12M, T14E, | IELEVEPSDTIENI | GATGATCGAGTTGGAAGTGGAGCCTTCCGAT |
| | K33H, A46Q, | KAKIQDHEGIPLD | ACTATCGAGAATATTAAGGCCAAAATCCAAG |
| | S65P, L67K, | QQRLTFQGKLLE | ATCATGAAGGGATTCCTTTGGATCAACAACG |
| | H68M, I61L, | DGRTLSDYNLTIS | CCTTACTTTTCAAGGGAAGTTGCTGGAGGAC |
| | K63I, A44T, | PEKMAVLRMQ | GGTCGCACACTGTCTGACTATAACTTGACTAT |
| | S49L, P38L, | | TTCTCCTGAGAAGATGGCCGTACTGCGCATG |
| | R74Q, M1Y, | | CAA |
| | V26I, L73M, | | |
| | P69A, L70V, | | |
| | L2T, L62T, | | |
| | D64S, K66E | | |
| CM493 | K6R, T7M, | YWIFVRMLTGK | TATTGGATTTTCGTACGCATGTTGACTGGAAA |
| [662; 879] | T12M, T14E, | MIELEVEPSDTIE | GATGATCGAGTTGGAAGTGGAGCCTTCCGAT |
| | K33H, A46Q, | NIKAKIQDHEGIP | ACTATCGAGAATATTAAGGCCAAAATCCAAG |
| | S65P, L67K, | LDQQRLTFQGKL | ATCATGAAGGGATTCCTTTGGATCAACAACG |
| | H68M, I61L, | LEDGRTLSDYNLT | CCTTACTTTTLAAGGGAAGTTGCTGGAGGAC |
| | K63I, A44T, | ISPEKMAVLRMQ | GGTCGCACACTGTCTGACTATAACTTGACTAT |
| | S49L, P38L, | | TTCTCCTGAGAAGATGGCCGTACTGCGCATG |
| | R74Q, M1Y, | | CAA |
| | V26I, L73M, | | |
| | P69A, L70V, | | |
| | L2W, L62T, | | |
| | D64S, K66E | | |
| CM494 | K6R, T7M, | YYIFVRMLTGKM | TATTATATTTTCGTACGCATGTTGACTGGAAA |
| [663; 880] | T12M, T14E, | IELEVEPSDTIENI | GATGATCGAGTTGGAAGTGGAGCCTTCCGAT |
| | K33H, A46Q, | KAKIQDHEGIPLD | ACTATCGAGAATATTAAGGCCAAAATCCAAG |
| | S65P, L67K, | QRLTFQGKLLE | ATCATGAAGGGATTCCTTTGGATCAACAACG |
| | H68M, I61L, | DGRTLSDYNLTIS | CCTTACTTTTCAAGGGAAGTTGCTGGAGGAC |
| | K63I, A44T, | PEKMAVLRMQ | GGTCGCACACTGTCTGACTATAACTTGACTAT |
| | S49L, P38L, | | TTCTCCTGAGAAGATGGCCGTACTGCGCATG |
| | R74Q, M1Y, | | CAA |
| | V26I, L73M, | | |
| | P69A, L70V, | | |
| | L2Y, L62T, | | |
| | D64S, K66E | | |
| CM495 | K6R, T7M, | YVIFVRMLTGKM | TATGTAATTTTCGTACGCATGTTGACTGGAAA |
| [664; 881] | T12M, T14E, | IELEVEPSDTIENI | GATGATCGAGTTGGAAGTGGAGCCTTCCGAT |
| | K33H, A46Q, | KAKIQDHEGIPLD | ACTATCGAGAATATTAAGGCCAAAATCCAAG |
| | S65P, L67K, | QQRLTFQGKLLE | ATCATGAAGGGATTCCTTTGGATCAACAACG |
| | H68M, I61L, | DGRTLSDYNLTIS | CCTTACTTTTCAAGGGAAGTTGCTGGAGGAC |
| | K63I, A44T, | PEKMAVLRMQ | GGTCGCACACTGTCTGACTATAACTTGACTAT |
| | S49L, P38L, | | TTCTCCTGAGAAGATGGCCGTACTGCGCATG |
| | R74Q, M1Y, | | CAA |
| | V26I, L73M, | | |
| | P69A, L70V, | | |
| | L2V, L62T, | | |
| | D64S, K66E | | |
| CM496 | K6R, T7M, | YPIFVRMLTGKM | TATCCTATTTTCGTACGCATGTTGACTGGAAA |
| [665; 882] | T12M, T14E, | IELEVEPSDTIENI | GATGATCGAGTTGGAAGTGGAGCCTTCCGAT |
| | K33H, A46Q, | KAKIQDHEGIPLD | ACTATCGAGAATATTAAGGCCAAAATCCAAG |
| | S65P, L67K, | QQRLTFQGKLLE | ATCATGAAGGGATTCCTTTGGATCAACAACG |
| | H68M, I61L, | DGRTLSDYNLTIS | CCTTACTTTTCAAGGGAAGTTGCTGGAGGAC |
| | K63I, A44T, | PEKMAVLRMQ | GGTCGCACACTGTCTGACTATAACTTGACTAT |
| | S49L, P38L, | | TTCTCCTGAGAAGATGGCCGTACTGCGCATG |
| | R74Q, M1Y, | | CAA |
| | V26I, L73M, | | |
| | P69A, L70V, | | |
| | L2P, L62T, | | |
| | D64S, K66E | | |

^{a}The SEQ ID NOS shown in brackets correspond to the protein amino acid SEQ ID NO, followed by the DNA nucleic acid SEQ ID NO.

### Definitions

To aid in understanding the invention, several terms are defined below.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The term “CRISPR” refers to Clustered Regularly Interspaced Short Palindromic Repeat bacterial adaptive immune system.

The terms “Cas” and “Cas endonuclease” generally refers to a CRISPR-associated endonuclease.

The term “Cas protein” generally refers to a wild-type protein, including a variant thereof, of a CRISPR-associated endonuclease (including the interchangeable terms Cas and Cas endonuclease).

The term “Cas nucleic acid” generally refers to a nucleic acid of a CRISPR-associated endonuclease, including a guide RNA, sgRNA, crRNA, or tracrRNA.

The terms “Cas9” and “CRISPR/Cas9” refer to the CRISPR-associated bacterial adaptive immune system of Steptococcus pyogenes. Examples of this system are disclosed in U.S. patent application Ser. Nos. 15/729,491 and 15/964,041, filed Oct. 10, 2017 and Apr. 26, 2018, respectively (Attorney Docket Nos. IDT01-009-US and IDT01-009-US-CIP, respectively), the contents of which are incorporated by reference herein.

The terms “AsCas12a” and “CRISPR/AsCas12a” refer to the CRISPR-associated bacterial adaptive immune system of Acidaminococcus sp. Examples of this system are disclosed in U.S. patent application Ser. No. 16/536,256, filed Aug. 8, 2019, (Attorney Docket No. IDT01-013-US), the contents of which are incorporated by reference herein.

The terms “LbCas12a” and “CRISPR/LbCas12a” refer to the CRISPR-associated bacterial adaptive immune system of Lachnospiraceae bacterium. Examples of this system are disclosed in U.S. Patent Application Ser. No. 63/018,592, filed May 1, 2020, (Attorney Docket No. IDT01-017-PRO), the contents of which are incorporated by reference herein.

The term “variant,” as that term modifies a protein (for example, ubiquitin), refers to a protein that includes at least one amino substitution of the reference, typically wild-type, protein amino acid sequence, additional amino acids (for example, such as an affinity tag or nuclear localization signal), or a combination thereof.

The term “polypeptide” refers to any linear or branched peptide comprising more than one amino acid. Polypeptide includes protein or fragment thereof or fusion thereof, provided such protein, fragment or fusion retains a useful biochemical or biological activity. In terms or manufacturing methods, “polypeptide” refers to synthetic polypeptides that may be produced from chemical means as well as polypeptides expressed from translation in vitro or in vivo.

The terms “fusion protein” and “fusion polypeptide” are interchangeable and typically includes extra amino acid information that is not native to the protein to which the extra amino acid information is covalently attached. Such extra amino acid information may include tags that enable purification or identification of the fusion protein. Such extra amino acid information may include peptides that enable the fusion proteins to be transported into cells and/or transported to specific locations within cells. Examples of tags for these purposes include affinity tags and nuclear localization signals (NLS), such as those obtained from SV40, allow for proteins to be transported to the nucleus immediately upon entering the cell. Given that the native Cas9 protein is bacterial in origin and therefore does not naturally comprise a NLS motif, addition of one or more NLS motifs to the recombinant Cas9 protein is expected to show improved genome editing activity when used in eukaryotic cells where the target genomic DNA substrate resides in the nucleus. One skilled in the art would appreciate these various fusion tag technologies, as well as how to make and use fusion proteins that include them

The terms “Ubiquitin” or “human Ubiquitin” refers to the wild-type Ubiquitin polypeptide amino acid sequence (SEQ ID NO:1).

The terms “i53,” i53 Ubiquitin,” or “Ubiquitin i53” refers to a ubiquitin variant polypeptide amino acid sequence (SEQ ID NO:2) that lacks the carboxy terminal di-glycine of the wild-type Ubiquitin polypeptide and includes several amino acid substitutions (Q2L, I44A, Q49S, Q62L, E64D, T66K, L69P, and V70L) relative to the wild-type Ubiquitin polypeptide.

The terms “polynucleotide” and “nucleic acid” are interchangeable and refer to synthetic DNA or synthetic RNA, including synthetic mRNA, as well as RNA, including mRNA that may be expressed from DNA or from a vector in vitro or in vivo. The SEQ ID NOS of polynucleotides have been presented in DNA forms without limiting that the corresponding RNA versions, including mRNA versions of those sequences may be readily deduces by one skilled in the art. Accordingly, while the SEQ ID NOS of polynucleotides formally define DNA sequences, such SEQ ID NOS implicitly encompass the RNA sequence counterparts of those DNA sequences as well.

One of ordinary skill in the art would appreciate that an isolated polypeptide or isolated polynucleotide comprising a particular SEQ ID NO will encompass the particular amino acid or nucleotide sequence defined by the SEQ ID NO as well as include any additional amino acid or nucleotide information not included within the given SEQ ID NO.

### REFERENCES

1. Chapman, J. R., M. R. Taylor, and S. J. Boulton, Playing the endgame: DNA double-strand break repair pathway choice. Mol Cell, 2012. 47(4): p. 497-510.
2. Iwabuchi, K., et al., Two cellular proteins that bind to wild-type but not mutant p53. Proc Natl Acad Sci USA, 1994. 91(13): p. 6098-102.
3. Escribano-Diaz, C., et al., A cell cycle-dependent regulatory circuit composed of 53BP1-RIF1 and BRCA1-CtIP controls DNA repair pathway choice. Mol Cell, 2013. 49(5): p. 872-83.
4. Feng, L., et al., RIF1 counteracts BRCA1-mediated end resection during DNA repair. J Biol Chem, 2013. 288(16): p. 11135-43.
5. Xie, A., et al., Distinct roles of chromatin-associated proteins MDC1 and 53BP1 in mammalian double-strand break repair. Mol Cell, 2007. 28(6): p. 1045-57.
6. Gaj, T., et al., Genome-Editing Technologies: Principles and Applications. Cold Spring Harb Perspect Biol, 2016. 8(12).
7. Botuyan, M. V., et al., Structural basis for the methylation state-specific recognition of histone H4-K20 by 53BP1 and Crb2 in DNA repair. Cell, 2006. 127(7): p. 1361-73.
8. Charier, G., et al., The Tudor tandem of 53BP: a new structural motif involved in DNA and RG-rich peptide binding. Structure, 2004. 12(9): p. 1551-62.
9. Fradet-Turcotte, A., et al., 53BP1 is a reader of the DNA-damage-induced H2A Lys 15 ubiquitin mark. Nature, 2013. 499(7456): p. 50-4.
10. Mattiroli, F., et al., RNF168 ubiquitinates K13-15 on H2A H2AX to drive DNA damage signaling. Cell, 2012. 150(6): p. 1182-95.
11. Canny, M. D., et al., Inhibition of 53BP1 favors homology-dependent DNA repair and increases CRISPR-Cas9 genome-editing efficiency. Nat Biotechnol, 2018. 36(1): p. 95-102.
12. Dikic, I., Wakatsuki, S. & Walters, K. J. Ubiquitin-binding domains—from structures to functions. Nature reviews. Molecular cell biology 10, 659-671 (2009).
13. Davis, L. and N. Maizels, Two Distinct Pathways Support Gene Correction by Single-StrandedDonors at DNA Nicks. Cell Rep, 2016. 17(7): p. 1872-1881.
14. Verma, P. and R. A. Greenberg, Noncanonical views of homology-directed DNA repair. Genes Dev, 2016. 30(10): p. 1138-54.
15. Butala, M., D. Zgur-Bertok, and S. J. Busby, The bacterial LexA transcriptional repressor. Cell Mol Life Sci, 2009. 66(1): p. 82-93.
16. Thliveris, A. T., J. W. Little, and D. W. Mount, Repression of the E coli recA gene requires at least two LexA protein monomers. Biochimie, 1991. 73(4): p. 449-56.
17. Thliveris, A. T. and D. W. Mount, Genetic identification ofthe DNA binding domain of Escherichia coli LexA protein. Proc Natl Acad Sci USA, 1992. 89(10): p. 4500-4.
18. Clarke, P., P. O. Cuiv, and M. O'Connell, Novel mobilizable prokaryotic two-hybrid system vectors for high-throughput protein interaction mapping in Escherichia coli by bacterial conjugation. Nucleic Acids Res, 2005. 33(2): p. e18.
19. Griffith, K. L. and R. E. Wolf, Jr., Measuring beta-galactosidase activity in bacteria: cell growth, permeabilization, and enzyme assays in 96-well arrays. Biochem Biophys Res Commun, 2002. 290(1): p. 397-402.
20. Wrenbeck, E. E., et al., Plasmid-based one-pot saturation mutagenesis. Nat Methods, 2016. 13(11): p. 928-930.
21. Ladant, D., Interaction of Bordetella pertussis adenylate cyclase with calmodulin. Identification of two separated calmodulin-binding domains. J Biol Chem, 1988. 263(6): p. 2612-8.
22. Ladant, D., et al., Characterization of the calmodulin-binding and of the catalytic domains of Bordetella pertussis adenylate cyclase. J Biol Chem, 1989. 264(7): p. 4015-20.
23. Karimova, G., et al., A bacterial two-hybrid system based on a reconstituted signal transduction pathway. Proc Natl Acad Sci USA, 1998. 95(10): p. 5752-6.
24. Datsenko, K. A. & Wanner, B. L. One-step inactivation of chromosomal genes in Escherichia coli K-12 using PCR products. Proc Natl Acad Sci USA 97, 6640-6645 (2000).
25. Rubin, A. F. et al. A statistical framework for analyzing deep mutational scanning data. Genome Biol 18, 150 (2017).
26. Yang, H. et al. Methods Favoring Homology-Directed Repair Choice in Response to CRISPR/Cas9 Induced-Double Strand Breaks. Int JMol Sci 21 (2020).
27. Fok, J. H. L. et al. AZD7648 is a potent and selective DNA-PK inhibitor that enhances radiation, chemotherapy and olaparib activity. Nat Commun 10, 5065 (2019).
28. Riesenberg, S. & Maricic, T. Targeting repair pathways with small molecules increases precise genome editing in pluripotent stem cells. Nat Commun 9, 2164 (2018).
29. Panier, S. & Boulton, S. J. Double-strand break repair: 53BP1 comes into focus. Nature reviews. Molecular cell biology 15, 7-18 (2014).
30. Callen, E. et al. 53BP1 mediates productive and mutagenic DNA repair through distinct phosphoprotein interactions. Cell 153, 1266-1280 (2013).
31. Yanai, M. et al. DNA-PK Inhibition by NU7441 Enhances Chemosensitivity to Topoisomerase Inhibitor in Non-Small Cell Lung Carcinoma Cells by Blocking DNA Damage Repair. Yonago Acta Med 60, 9-15 (2017).
32. Jimeno, S. et al. Neddylation inhibits CtIP-mediated resection and regulates DNA double strand break repair pathway choice. Nucleic Acids Res 43, 987-999 (2015).
33. Bertino, E. M. & Otterson, G. A. Romidepsin: a novel histone deacetylase inhibitor for cancer. Expert Opinion on Investigational Drugs 20, 1151-1158 (2011).
34. Zhang, J. P. et al. HDAC inhibitors improve CRISPR-mediated HDR editing efficiency in iPSCs. Sci China Life Sci 64, 1449-1462 (2021).
35. Li, G. et al. Increasing CRISPR/Cas9-mediated homology-directed DNA repair by histone deacetylase inhibitors. Int J Biochem Cell Biol 125, 105790 (2020).
36. Tang, J. et al. Acetylation limits 53BP1 association with damaged chromatin to promote homologous recombination. Nat Struct Mol Biol 20, 317-325 (2013).
37. Hsiao, K. Y. & Mizzen, C. A. Histone H4 deacetylation facilitates 53BP1 DNA damage signaling and double-strand break repair. J Mol Cell Biol 5, 157-165 (2013).
38. Chapman, J. R. et al. RIF1 is essential for 53BP1-dependent nonhomologous end joining and suppression of DNA double-strand break resection. Molecular cell 49, 858-871 (2013).
39. Mallette, F. A. et al. RNF8- and RNF168-dependent degradation of KDM4A/JMJD2A triggers 53BP1 recruitment to DNA damage sites. Embo j 31, 1865-1878 (2012).
40. Ma, T. et al. RNF 111-dependent neddylation activates DNA damage-induced ubiquitination. Molecular cell 49, 897-907 (2013).
41. Brault, J. et al. CRISPR-targeted MAGT1 insertion restores XMEN patient hematopoietic stem cells and lymphocytes. Blood 138, 2768-2780 (2021).
42. De Ravin, S. S. et al. Enhanced homology-directed repair for highly efficient gene editing in hematopoietic stem/progenitor cells. Blood 137, 2598-2608 (2021).
43. Sweeney, C. L. et al. Correction of X-CGD patient HSPCs by targeted CYBB cDNA insertion using CRISPR/Cas9 with 53BP1 inhibition for enhanced homology-directed repair. Gene Ther 28, 373-390 (2021).
44. Wienert, B. et al. Timed inhibition of CDC7 increases CRISPR-Cas9 mediated templated repair. Nat Commun 11, 2109 (2020).

All references, including publications, patent applications, and patents, cited herein are hereby incorporated by reference to the same extent as if each reference were individually and specifically indicated to be incorporated by reference and were set forth in its entirety herein.

Preferred embodiments of this invention are described herein, including the best mode known to the inventors for carrying out the invention. Variations of those preferred embodiments may become apparent to those of ordinary skill in the art upon reading the foregoing description.

The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. An isolated polypeptide comprising a ubiquitin polypeptide variant selected from one of the following:
SEQ ID NO:450, wherein X₁ is selected from M, H, Y, W, Q, T, F, S, R, I, and N; X₂ is selected from Q, L, I, and M; X₆ is selected from K and R; X₇ is selected from T, M, I, C, L, and V; X₉ is selected from T, I, S, E and V; X₁₂ is selected from T, M, and Y; X₁₃ is selected from I, F, H and P; X₁₄ is selected from T, E, D, H, and N; X₁₆ is selected from E, M, T, N, Y, D, and H; X₁₇ is selected from V and C; X₁₈ is selected from E, M, Y, L, H, F, W, S, Q, T, C, N, R, and D; X₁₉ is selected from P and K; X₂₀ is selected from S, D, N, C, A, and W; X₂₁ is selected from D and E; X₂₅ is selected from N, V, I, E, G, M, Q, D, A, L, R, S, K, T, C, and F; X₂₆ is selected from I, V, and L; X₂₈ is selected from A, E, Q, W, I, M, and D; X₂₉ is selected from K, M, L, R, Q, and H; X₃1 is selected from Q, C, F, W, H, Y, L, R, and M; X₃₂ is selected from D, A, E, and R; X₃₃ is selected from K, H, A, Q, S, V, L, E, M, T, I, F, C, Y, R, N, and W; X₃₄ is selected from E and T; X₃₈ is selected from P, L, C, F, I, V, Y, T, M, H, S, Q, A, W, N, and K; X₃₉ is selected from D, W, E, G, S, L, and Q; X₄₀ is selected from Q, E, and D; X₄₁ is selected from Q, Y, I, C, and V; X₄₂ is selected from R, W, F, H, Y, N, C, and S; X₄₄ is selected from I, A and T; X₄₆ is selected from A, Q, and G; X₄₈ is selected from K, T, M, I, Q, V, R, L, and N; X₄₉ is selected from Q, S, L, M, P, E V, A, D, I, C, G, and N; X₅₁ is selected from E and D; X₅₂ is selected from D and E; X₅₄ is selected from R, Y, M, T, H, F, N, Q, K, and C; X₅₅ is selected from T and R; X₅₇ is selected from S, G, D, N, H, E, A, Q, M, R, and K; X₅₈ is selected from D and S; X₆₀ is selected from N, E, and Q; X₆₁ is selected from I and L; X₆₂ is selected from Q, L, T, V, C, A, M, I and S; X₆₃ is selected from K, I, M, F, and V; X₆₄ is selected from E, D, and S; X₆₅ is selected from S, P, E, K, H, R, A, D, N, and Q; X₆₆ is selected from T, K, R, and E; X₆₇ is selected from L, H, K, R, S, M, C, Y, and T; X₆₈ is selected from H, M, Q, and E; X₆₉ is selected from L, P, R, A, G, C, F, M, and S; X₇₀ is selected from V, L, M, F, and C; X₇₃ is selected from L and M; and X₇₄ is selected from R, Q, V, L, M, C, I, T, E, and K, and combinations thereof, provided that SEQ ID NOS:1-3 are excluded; and
at least one member selected from the group of SEQ ID NOs:452-665.

2. The isolated polypeptide according to claim 1, wherein isolated polypeptide comprises a ubiquitin polypeptide variant selected from SEQ ID NO:450,
wherein X₁ is selected from M, H, Y, W, Q, T, F, S, R, I, and N; X₂ is selected from Q, L, I, and M; X₆ is selected from K and R; X₇ is selected from T, M, I, C, L, and V; X₉ is selected from T, I, S, E and V; X₁₂ is selected from T, M, and Y; X₁₃ is selected from I, F, H and P; X₁₄ is selected from T, E, D, H, and N; X₁₆ is selected from E, M, T, N, Y, D, and H; X₁₇ is selected from V and C; X₁₈ is selected from E, M, Y, L, H, F, W, S, Q, T, C, N, R, and D; X₁₉ is selected from P and K; X₂₀ is selected from S, D, N, C, A, and W; X₂₁ is selected from D and E; X₂₅ is selected from N, V, I, E, G, M, Q, D, A, L, R, S, K, T, C, and F; X₂₆ is selected from I, V, and L; X₂₈ is selected from A, E, Q, W, I, M, and D; X₂₉ is selected from K, M, L, R, Q, and H; X₃₁ is selected from Q, C, F, W, H, Y, L, R, and M; X₃₂ is selected from D, A, E, and R; X₃₃ is selected from K, H, A, Q, S, V, L, E, M, T, I, F, C, Y, R, N, and W; X₃₄ is selected from E and T; X₃₈ is selected from P, L, C, F, I, V, Y, T, M, H, S, Q, A, W, N, and K; X₃₉ is selected from D, W, E, G, S, L, and Q; X₄₀ is selected from Q, E, and D; X₄₁ is selected from Q, Y, I, C, and V; X₄₂ is selected from R, W, F, H, Y, N, C, and S; X₄₄ is selected from I, A and T; X₄₆ is selected from A, Q, and G; X₄₈ is selected from K, T, M, I, Q, V, R, L, and N; X₄₉ is selected from Q, S, L, M, P, E V, A, D, I, C, G, and N; X₅₁ is selected from E and D; X₅₂ is selected from D and E; X₅₄ is selected from R, Y, M, T, H, F, N, Q, K, and C; X₅₅ is selected from T and R; X₅₇ is selected from S, G, D, N, H, E, A, Q, M, R, and K; X₅₈ is selected from D and S; X₆₀ is selected from N, E, and Q; X₆₁ is selected from I and L; X₆₂ is selected from Q, L, T, V, C, A, M, I and S; X₆₃ is selected from K, I, M, F, and V; X₆₄ is selected from E, D, and S; X₆₅ is selected from S, P, E, K, H, R, A, D, N, and Q; X₆₆ is selected from T, K, R, and E; X₆₇ is selected from L, H, K, R, S, M, C, Y, and T; X₆₈ is selected from H, M, Q, and E; X₆₉ is selected from L, P, R, A, G, C, F, M, and S; X₇₀ is selected from V, L, M, F, and C; X₇₃ is selected from L and M; and X₇₄ is selected from R, Q, V, L, M, C, I, T, E, and K, and combinations thereof, provided that SEQ ID NOS:1-3 are excluded.

3. The isolated polypeptide according to claim 2, wherein the isolated polypeptide shares amino acid sequence identity in the range of at least 40% to 100% identity of SEQ ID NO:1.

4. The isolated polypeptide according to claim 2, wherein the isolated polypeptide shares amino acid sequence identity in the range of at least 50% to 100% identity of SEQ ID NO:1.

5. The isolated polypeptide according to claim 2, wherein the isolated polypeptide shares amino acid sequence identity in the range of at least 60% to 100% identity of SEQ ID NO:1.

6. The isolated polypeptide according to claim 2, wherein the isolated polypeptide shares amino acid sequence identity in the range of at least 70% to 100% identity of SEQ ID NO:1.

7. The isolated polypeptide according to claim 2, wherein the isolated polypeptide shares amino acid sequence identity in the range of at least 80% to 100% identity of SEQ ID NO:1.

8. The isolated polypeptide according to claim 2, wherein the isolated polypeptide shares amino acid sequence identity in the range of at least 90% to 100% identity of SEQ ID NO:1.

9. The isolated polypeptide according to claim 2, wherein the isolated polypeptide shares amino acid sequence identity in the range of at least 95% to 100% identity of SEQ ID NO:1.

10. An isolated polypeptide comprising an isolated fusion polypeptide having an Ubv amino acid sequence with an N-terminal His₆-tag, wherein the isolated fusion polypeptide comprises at least one member selected from the following:
an isolated fusion polypeptide comprising SEQ ID NO: 1100, wherein X₁₂ is selected from M, H, Y, W, Q, T, F, S, R, I, and N; X₁₃ is selected from Q, L, I, and M; X₁₇ is selected from K and R; X₁₈ is selected from T, M, I, C, L, and V; X₂₀ is selected from T, I, S, E and V; X₂₃ is selected from T, M, and Y; X₂₄ is selected from I, F, H and P; X₂₅ is selected from T, E, D, H, and N; X₂₇ is selected from E, M, T, N, Y, D, and H; X₂₈ is selected from V and C; X₂₉ is selected from E, M, Y, L, H, F, W, S, Q, T, C, N, R, and D; X₃₀ is selected from P and K; X₃₁ is selected from S, D, N, C, A, and W; X₃₂ is selected from D and E; X₃₆ is selected from N, V, I, E, G, M, Q, D, A, L, R, S, K, T, C, and F; X₃₇ is selected from I, V, and L; X₃₉ is selected from A, E, Q, W, I, M, and D; X₄₀ is selected from K, M, L, R, Q, and H; X₄₂ is selected from Q, C, F, W, H, Y, L, R, and M; X₄₃ is selected from D, A, E, and R; X₄₄ is selected from K, H, A, Q, S, V, L, E, M, T, I, F, C, Y, R, N, and W; X₄₅ is selected from E and T; X₄₉ is selected from P, L, C, F, I, V, Y, T, M, H, S, Q, A, W, N, and K; X₅₀ is selected from D, W, E, G, S, L, and Q; X₅₁ is selected from Q, E, and D; X₅₂ is selected from Q, Y, I, C, and V; X₅₃ is selected from R, W, F, H, Y, N, C, and S; X₅₅ is selected from I, A and T; X₅₇ is selected from A, Q, and G; X₅₉ is selected from K, T, M, I, Q, V, R, L, and N; X₆₀ is selected from Q, S, L, M, P, E V, A, D, I, C, G, and N; X₆₂ is selected from E and D; X₆₃ is selected from D and E; X₆₅ is selected from R, Y, M, T, H, F, N, Q, K, and C; X₆₆ is selected from T and R; X₆₈ is selected from S, G, D, N, H, E, A, Q, M, R, and K; X₆₉ is selected from D and S; X₇₁ is selected from N, E, and Q; X₇₂ is selected from I and L; X₇₃ is selected from Q, L, T, V, C, A, M, I and S; X₇₄ is selected from K, I, M, F, and V; X₇₅ is selected from E, D, and S; X₇₆ is selected from S, P, E, K, H, R, A, D, N, and Q; X₇₇ is selected from T, K, R, and E; X₇₈ is selected from L, H, K, R, S, M, C, Y, and T; X₇₉ is selected from H, M, Q, and E; X₈₀ is selected from L, P, R, A, G, C, F, M, and S; X₈₁ is selected from V, L, M, F, and C; X₈₄ is selected from L and M; and X₈₅ is selected from R, Q, V, L, M, C, I, T, E, and K, and combinations thereof, provided that SEQ ID NO: 3 is excluded; and
an isolated fusion polypeptide comprising at least one member selected SEQ ID NOS:235-244 and 246-449.

11. The isolated polypeptide of claim 10, wherein the isolated fusion polypeptide comprises SEQ ID NO: 1100, wherein X₁₂ is selected from M, H, Y, W, Q, T, F, S, R, I, and N; X₁₃ is selected from Q, L, I, and M; X₁₇ is selected from K and R; X₁₈ is selected from T, M, I, C, L, and V; X₂₀ is selected from T, I, S, E and V; X₂₃ is selected from T, M, and Y; X₂₄ is selected from I, F, H and P; X₂₅ is selected from T, E, D, H, and N; X₂₇ is selected from E, M, T, N, Y, D, and H; X₂₈ is selected from V and C; X₂₉ is selected from E, M, Y, L, H, F, W, S, Q, T, C, N, R, and D; X₃₀ is selected from P and K; X₃₁ is selected from S, D, N, C, A, and W; X₃₂ is selected from D and E; X₃₆ is selected from N, V, I, E, G, M, Q, D, A, L, R, S, K, T, C, and F; X₃₇ is selected from I, V, and L; X₃₉ is selected from A, E, Q, W, I, M, and D; X₄₀ is selected from K, M, L, R, Q, and H; X₄₂ is selected from Q, C, F, W, H, Y, L, R, and M; X₄₃ is selected from D, A, E, and R; X₄₄ is selected from K, H, A, Q, S, V, L, E, M, T, I, F, C, Y, R, N, and W; X₄₅ is selected from E and T; X₄₉ is selected from P, L, C, F, I, V, Y, T, M, H, S, Q, A, W, N, and K; X₅₀ is selected from D, W, E, G, S, L, and Q; X₅₁ is selected from Q, E, and D; X₅₂ is selected from Q, Y, I, C, and V; X₅₃ is selected from R, W, F, H, Y, N, C, and S; X₅₅ is selected from I, A and T; X₅₇ is selected from A, Q, and G; X₅₉ is selected from K, T, M, I, Q, V, R, L, and N; X₆₀ is selected from Q, S, L, M, P, E V, A, D, I, C, G, and N; X₆₂ is selected from E and D; X₆₃ is selected from D and E; X₆₅ is selected from R, Y, M, T, H, F, N, Q, K, and C; X₆₆ is selected from T and R; X₆₈ is selected from S, G, D, N, H, E, A, Q, M, R, and K; X₆₉ is selected from D and S; X₇₁ is selected from N, E, and Q; X₇₂ is selected from I and L; X₇₃ is selected from Q, L, T, V, C, A, M, I and S; X₇₄ is selected from K, I, M, F, and V; X₇₅ is selected from E, D, and S; X₇₆ is selected from S, P, E, K, H, R, A, D, N, and Q; X₇₇ is selected from T, K, R, and E; X₇₈ is selected from L, H, K, R, S, M, C, Y, and T; X₇₉ is selected from H, M, Q, and E; X₈₀ is selected from L, P, R, A, G, C, F, M, and S; X₈₁ is selected from V, L, M, F, and C; X₈₄ is selected from L and M; and X₈₅ is selected from R, Q, V, L, M, C, I, T, E, and K, and combinations thereof, provided that SEQ ID NO: 3 is excluded.

12. The isolated polypeptide according to claim 11, wherein the isolated polypeptide of SEQ ID 1100 encompassing amino acid positions 12-85 shares amino acid sequence identity in the range of at least 40% to 100% identity of SEQ ID NO:1.

13. The isolated polypeptide according to claim 11, wherein the isolated polypeptide of SEQ ID 1100 encompassing amino acid positions 12-85 shares amino acid sequence identity in the range of at least 50% to 100% identity of SEQ ID NO:1.

14. The isolated polypeptide according to claim 11, wherein the isolated polypeptide of SEQ ID 1100 encompassing amino acid positions 12-85 shares amino acid sequence identity in the range of at least 60% to 100% identity of SEQ ID NO:1.

15. The isolated polypeptide according to claim 11, wherein the isolated polypeptide of SEQ ID 1100 encompassing amino acid positions 12-85 shares amino acid sequence identity in the range of at least 70% to 100% identity of SEQ ID NO:1.

16. The isolated polypeptide according to claim 11, wherein the isolated polypeptide of SEQ ID 1100 encompassing amino acid positions 12-85 shares amino acid sequence identity in the range of at least 80% to 100% identity of SEQ ID NO:1.

17. The isolated polypeptide according to claim 11, wherein the isolated polypeptide of SEQ ID 1100 encompassing amino acid positions 12-85 shares amino acid sequence identity in the range of at least 90% to 100% identity of SEQ ID NO:1.

18. The isolated polypeptide according to claim 11, wherein the isolated polypeptide of SEQ ID 1100 encompassing amino acid positions 12-85 shares amino acid sequence identity in the range of at least 95% to 100% identity of SEQ ID NO:1.

19. An isolated polypeptide with enhanced HDR activity through interactions with 53BP1 in a manner to influence repair mechanisms at DSB sites, comprising:
an isolated polypeptide comprising a Ubv having at least 40% amino acid sequence identity to amino acid positions 1-74 of SEQ ID NOS:1, 2, 482, 633, or 450, provided that SEQ ID NOS:1 and 2 are excluded, and those having at least 40% amino acid sequence identity with amino acid positions 12-85 of SEQ ID NOS: 3, 241, 417, or 1100, provided that SEQ ID NO:3 is excluded,
wherein isolated polypeptide provides enhanced HDR activity through interactions with 53BP1 in a manner to influence repair mechanisms at DSB sites relative to SEQ ID NO:1 under identical conditions.

20. The isolated polypeptide of claim 19, wherein the isolated polypeptide comprising a Ubv having at least 50% amino acid sequence identity to amino acid positions 1-74 of SEQ ID NOS:1, 2, 482, 633, or 450, provided that SEQ ID NOS:1 and 2 are excluded, and those having at least 50% amino acid sequence identity with amino acid positions 12-85 of SEQ ID NOS: 3, 241, 417, or 1100, provided that SEQ ID NO:3 is excluded.

21. The isolated polypeptide of claim 19, wherein the isolated polypeptide comprising a Ubv having at least 60% amino acid sequence identity to amino acid positions 1-74 of SEQ ID NOS:1, 2, 482, 633, or 450, provided that SEQ ID NOS:1 and 2 are excluded, and those having at least 60% amino acid sequence identity with amino acid positions 12-85 of SEQ ID NOS: 3, 241, 417, or 1100, provided that SEQ ID NO:3 is excluded.

22. The isolated polypeptide of claim 19, wherein the isolated polypeptide comprising a Ubv having at least 70% amino acid sequence identity to amino acid positions 1-74 of SEQ ID NOS:1, 2, 482, 633, or 450, provided that SEQ ID NOS:1 and 2 are excluded, and those having at least 70% amino acid sequence identity with amino acid positions 12-85 of SEQ ID NOS: 3, 241, 417, or 1100, provided that SEQ ID NO:3 is excluded.

23. The isolated polypeptide of claim 19, wherein the isolated polypeptide comprising a Ubv having at least 80% amino acid sequence identity to amino acid positions 1-74 of SEQ ID NOS:1, 2, 482, 633, or 450, provided that SEQ ID NOS:1 and 2 are excluded, and those having at least 80% amino acid sequence identity with amino acid positions 12-85 of SEQ ID NOS: 3, 241, 417, or 1100, provided that SEQ ID NO:3 is excluded.

24. The isolated polypeptide of claim 19, wherein the isolated polypeptide comprising a Ubv having at least 90% amino acid sequence identity to amino acid positions 1-74 of SEQ ID NOS:1, 2, 482, 633, or 450, provided that SEQ ID NOS:1 and 2 are excluded, and those having at least 90% amino acid sequence identity with amino acid positions 12-85 of SEQ ID NOS: 3, 241, 417, or 1100, provided that SEQ ID NO:3 is excluded.

25. The isolated polypeptide of claim 19, wherein the isolated polypeptide comprising a Ubv having at least 95% amino acid sequence identity to amino acid positions 1-74 of SEQ ID NOS:1, 2, 482, 633, or 450, provided that SEQ ID NOS:1 and 2 are excluded, and those having at least 95% amino acid sequence identity with amino acid positions 12-85 of SEQ ID NOS: 3, 241, 417, or 1100, provided that SEQ ID NO:3 is excluded.

26. An isolated polynucleotide that encodes the isolated polypeptide of any of claims 19-26.

27. An isolated polynucleotide encoding a ubiquitin polypeptide variant, wherein the isolated polynucleotide comprises at least one member selected from SEQ ID NOS:669-682, 885-890, and 892-1099, and the corresponding RNA counterparts thereof.

28. A vector comprising an isolated polynucleotide encoding a ubiquitin polypeptide variant, wherein the isolated polynucleotide comprises at least one member selected from SEQ ID NOS:669-682, 885-890, and 892-1099, and the corresponding RNA counterparts thereof.

29. A cell or cell line comprising the isolated polypeptide from any of claims 1-26, the isolated polynucleotide of claim 27 or 28, or the vector of claim 29.

30. A method of suppressing 53BP1 recruitment to DNA double-strand break sites in a cell, comprising:
administering to the cell the isolated polypeptide from any of claims 1-26, the isolated polynucleotide of claim 27 or 28, or the vector of claim 29.

31. A method of increasing homologous recombination in a cell comprising:
administering to the cell the isolated polypeptide from any of claims 1-26, the isolated polynucleotide of claim 27 or 28, or the vector of claim 29.

32. A method of editing a gene in a cell using a CRISPR system, comprising:
administering to the cell the isolated polypeptide from any of claims 1-26, the isolated polynucleotide of claim 27 or 28, or the vector of claim 29.

33. A method of gene targeting in a cell, comprising:
administering to the cell isolated polypeptide from any of claims 1-26, the isolated polynucleotide of claim 27 or 28, or the vector of claim 29.

34. A composition comprising the isolated polypeptide of any of claims 1-26 in admixture with a carrier, excipient or diluent.

35. A composition comprising the isolated polypeptide of any of claims 1-26 and one or more components of a gene editing system.

36. A kit comprising the isolated polypeptide from any of claims 1-26, the isolated polynucleotide of claim 27 or 28, or the vector of claim 29.

37. The kit of claim 36, further comprising one or more components of a gene editing system.

38. The kit of claim 37, wherein the gene editing system is a CRISPR system.

39. A method of performing a medically therapeutic procedure, wherein the method includes the step of performing genome editing according to claim 33 or 34.

40. A method of screening for amino acid changes in a first polypeptide that improve affinity of the first polypeptide for a second polypeptide, comprising:
using the BACTH system with a reporter gene tinder control of cAMP regulated promoter to allow fluorescence activated cell sorting based on protein-protein interaction affinity between the first polypeptide and the second polypeptide to screen for improved affinity variants of the first polypeptide.


SEQUENCE LISTING (1112 sequences)

SEQ ID NO: 1 - protein, 76 aa, Homo sapiens [sequence omitted]

SEQ ID NO: 2 - protein, 74 aa, synthetic construct
MLIFVKTLTGKTITLEVEPSDTIENVKAKIQDKEGIPPDQQRLAFAGKSLEDGRTLSDYNILKDSKLHPLLRLR

SEQ ID NO: 3 - protein, 85 aa, synthetic construct
MHHHHHHGGSGMLIFVKTLTGKTITLEVEPSDTIENVKAKIQDKEGIPPDQQRLAFAGKSLEDGRTLSDYNILKDSKLHPLLRLR

SEQ ID NO: 4 - protein, 74 aa, synthetic construct
HLIFVKTLTGKTITLEVEPSDTIENVKAKIQDKEGIPPDQQRLAFAGKSLEDGRTLSDYNILKDSKLHPLLRLR

SEQ ID NO: 5 - protein, 74 aa, synthetic construct
YLIFVKTLTGKTITLEVEPSDTIENVKAKIQDKEGIPPDQQRLAFAGKSLEDGRTLSDYNILKDSKLHPLLRLR

SEQ ID NO: 6 - protein, 74 aa, synthetic construct
WLIFVKTLTGKTITLEVEPSDTIENVKAKIQDKEGIPPDQQRLAFAGKSLEDGRTLSDYNILKDSKLHPLLRLR

SEQ ID NO: 7 - protein, 74 aa, synthetic construct
QLIFVKTLTGKTITLEVEPSDTIENVKAKIQDKEGIPPDQQRLAFAGKSLEDGRTLSDYNILKDSKLHPLLRLR

SEQ ID NO: 8 - protein, 74 aa, synthetic construct
TLIFVKTLTGKTITLEVEPSDTIENVKAKIQDKEGIPPDQQRLAFAGKSLEDGRTLSDYNILKDSKLHPLLRLR

SEQ ID NO: 9 - protein, 74 aa, synthetic construct
FLIFVKTLTGKTITLEVEPSDTIENVKAKIQDKEGIPPDQQRLAFAGKSLEDGRTLSDYNILKDSKLHPLLRLR

SEQ ID NO: 10 - protein, 74 aa, synthetic construct
SLIFVKTLTGKTITLEVEPSDTIENVKAKIQDKEGIPPDQQRLAFAGKSLEDGRTLSDYNILKDSKLHPLLRLR

SEQ ID NO: 11 - protein, 74 aa, synthetic construct
ILIFVKTLTGKTITLEVEPSDTIENVKAKIQDKEGIPPDQQRLAFAGKSLEDGRTLSDYNILKDSKLHPLLRLR

SEQ ID NO: 12 - protein, 74 aa, synthetic construct
RLIFVKTLTGKTITLEVEPSDTIENVKAKIQDKEGIPPDQQRLAFAGKSLEDGRTLSDYNILKDSKLHPLLRLR

SEQ ID NO: 13 - protein, 74 aa, synthetic construct
NLIFVKTLTGKTITLEVEPSDTIENVKAKIQDKEGIPPDQQRLAFAGKSLEDGRTLSDYNILKDSKLHPLLRLR

SEQ ID NO: 14 - protein, 74 aa, synthetic construct
MLIFVRTLTGKTITLEVEPSDTIENVKAKIQDKEGIPPDQQRLAFAGKSLEDGRTLSDYNILKDSKLHPLLRLR

SEQ ID NO: 15 - protein, 74 aa, synthetic construct
MLIFVKMLTGKTITLEVEPSDTIENVKAKIQDKEGIPPDQQRLAFAGKSLEDGRTLSDYNILKDSKLHPLLRLR

SEQ ID NO: 16 - protein, 74 aa, synthetic construct
MLIFVKILTGKTITLEVEPSDTIENVKAKIQDKEGIPPDQQRLAFAGKSLEDGRTLSDYNILKDSKLHPLLRLR

SEQ ID NO: 17 - protein, 74 aa, synthetic construct
MLIFVKCLTGKTITLEVEPSDTIENVKAKIQDKEGIPPDQQRLAFAGKSLEDGRTLSDYNILKDSKLHPLLRLR

SEQ ID NO: 18 - protein, 74 aa, synthetic construct
MLIFVKLLTGKTITLEVEPSDTIENVKAKIQDKEGIPPDQQRLAFAGKSLEDGRTLSDYNILKDSKLHPLLRLR

SEQ ID NO: 19 - protein, 74 aa, synthetic construct
MLIFVKVLTGKTITLEVEPSDTIENVKAKIQDKEGIPPDQQRLAFAGKSLEDGRTLSDYNILKDSKLHPLLRLR

SEQ ID NO: 20 - protein, 74 aa, synthetic construct
MLIFVKTLSGKTITLEVEPSDTIENVKAKIQDKEGIPPDQQRLAFAGKSLEDGRTLSDYNILKDSKLHPLLRLR

SEQ ID NO: 21 - protein, 74 aa, synthetic construct
MLIFVKTLIGKTITLEVEPSDTIENVKAKIQDKEGIPPDQQRLAFAGKSLEDGRTLSDYNILKDSKLHPLLRLR

SEQ ID NO: 22 - protein, 74 aa, synthetic construct
MLIFVKTLEGKTITLEVEPSDTIENVKAKIQDKEGIPPDQQRLAFAGKSLEDGRTLSDYNILKDSKLHPLLRLR

SEQ ID NO: 23 - protein, 74 aa, synthetic construct
MLIFVKTLVGKTITLEVEPSDTIENVKAKIQDKEGIPPDQQRLAFAGKSLEDGRTLSDYNILKDSKLHPLLRLR

SEQ ID NO: 24 - protein, 74 aa, synthetic construct
MLIFVKTLTGKMITLEVEPSDTIENVKAKIQDKEGIPPDQQRLAFAGKSLEDGRTLSDYNILKDSKLHPLLRLR

SEQ ID NO: 25 - protein, 74 aa, synthetic construct
MLIFVKTLTGKYITLEVEPSDTIENVKAKIQDKEGIPPDQQRLAFAGKSLEDGRTLSDYNILKDSKLHPLLRLR

SEQ ID NO: 26 - protein, 74 aa, synthetic construct
MLIFVKTLTGKTFTLEVEPSDTIENVKAKIQDKEGIPPDQQRLAFAGKSLEDGRTLSDYNILKDSKLHPLLRLR

SEQ ID NO: 27 - protein, 74 aa, synthetic construct
MLIFVKTLTGKTHTLEVEPSDTIENVKAKIQDKEGIPPDQQRLAFAGKSLEDGRTLSDYNILKDSKLHPLLRLR

SEQ ID NO: 28 - protein, 74 aa, synthetic construct
MLIFVKTLTGKTPTLEVEPSDTIENVKAKIQDKEGIPPDQQRLAFAGKSLEDGRTLSDYNILKDSKLHPLLRLR

SEQ ID NO: 29 - protein, 74 aa, synthetic construct
MLIFVKTLTGKTIDLEVEPSDTIENVKAKIQDKEGIPPDQQRLAFAGKSLEDGRTLSDYNILKDSKLHPLLRLR

SEQ ID NO: 30 - protein, 74 aa, synthetic construct
MLIFVKTLTGKTIELEVEPSDTIENVKAKIQDKEGIPPDQQRLAFAGKSLEDGRTLSDYNILKDSKLHPLLRLR

SEQ ID NO: 31 - protein, 74 aa, synthetic construct
MLIFVKTLTGKTINLEVEPSDTIENVKAKIQDKEGIPPDQQRLAFAGKSLEDGRTLSDYNILKDSKLHPLLRLR

SEQ ID NO: 32 - protein, 74 aa, synthetic construct
MLIFVKTLTGKTIHLEVEPSDTIENVKAKIQDKEGIPPDQQRLAFAGKSLEDGRTLSDYNILKDSKLHPLLRLR

SEQ ID NO: 33 - protein, 74 aa, synthetic construct
MLIFVKTLTGKTITLTVEPSDTIENVKAKIQDKEGIPPDQQRLAFAGKSLEDGRTLSDYNILKDSKLHPLLRLR

SEQ ID NO: 34 - protein, 74 aa, synthetic construct
MLIFVKTLTGKTITLMVEPSDTIENVKAKIQDKEGIPPDQQRLAFAGKSLEDGRTLSDYNILKDSKLHPLLRLR

SEQ ID NO: 35 - protein, 74 aa, synthetic construct
MLIFVKTLTGKTITLYVEPSDTIENVKAKIQDKEGIPPDQQRLAFAGKSLEDGRTLSDYNILKDSKLHPLLRLR

SEQ ID NO: 36 - protein, 74 aa, synthetic construct
MLIFVKTLTGKTITLHVEPSDTIENVKAKIQDKEGIPPDQQRLAFAGKSLEDGRTLSDYNILKDSKLHPLLRLR

SEQ ID NO: 37 - protein, 74 aa, synthetic construct
MLIFVKTLTGKTITLNVEPSDTIENVKAKIQDKEGIPPDQQRLAFAGKSLEDGRTLSDYNILKDSKLHPLLRLR

SEQ ID NO: 38 - protein, 74 aa, synthetic construct
MLIFVKTLTGKTITLDVEPSDTIENVKAKIQDKEGIPPDQQRLAFAGKSLEDGRTLSDYNILKDSKLHPLLRLR

SEQ ID NO: 39 - protein, 74 aa, synthetic construct
MLIFVKTLTGKTITLECEPSDTIENVKAKIQDKEGIPPDQQRLAFAGKSLEDGRTLSDYNILKDSKLHPLLRLR

SEQ ID NO: 40 - protein, 74 aa, synthetic construct
MLIFVKTLTGKTITLEVYPSDTIENVKAKIQDKEGIPPDQQRLAFAGKSLEDGRTLSDYNILKDSKLHPLLRLR

SEQ ID NO: 41 - protein, 74 aa, synthetic construct
MLIFVKTLTGKTITLEVMPSDTIENVKAKIQDKEGIPPDQQRLAFAGKSLEDGRTLSDYNILKDSKLHPLLRLR

SEQ ID NO: 42 - protein, 74 aa, synthetic construct
MLIFVKTLTGKTITLEVQPSDTIENVKAKIQDKEGIPPDQQRLAFAGKSLEDGRTLSDYNILKDSKLHPLLRLR

SEQ ID NO: 43 - protein, 74 aa, synthetic construct
MLIFVKTLTGKTITLEVHPSDTIENVKAKIQDKEGIPPDQQRLAFAGKSLEDGRTLSDYNILKDSKLHPLLRLR

SEQ ID NO: 44 - protein, 74 aa, synthetic construct
MLIFVKTLTGKTITLEVFPSDTIENVKAKIQDKEGIPPDQQRLAFAGKSLEDGRTLSDYNILKDSKLHPLLRLR

SEQ ID NO: 45 - protein, 74 aa, synthetic construct
MLIFVKTLTGKTITLEVWPSDTIENVKAKIQDKEGIPPDQQRLAFAGKSLEDGRTLSDYNILKDSKLHPLLRLR

SEQ ID NO: 46 - protein, 74 aa, synthetic construct
MLIFVKTLTGKTITLEVLPSDTIENVKAKIQDKEGIPPDQQRLAFAGKSLEDGRTLSDYNILKDSKLHPLLRLR

SEQ ID NO: 47 - protein, 74 aa, synthetic construct
MLIFVKTLTGKTITLEVSPSDTIENVKAKIQDKEGIPPDQQRLAFAGKSLEDGRTLSDYNILKDSKLHPLLRLR

SEQ ID NO: 48 - protein, 74 aa, synthetic construct
MLIFVKTLTGKTITLEVRPSDTIENVKAKIQDKEGIPPDQQRLAFAGKSLEDGRTLSDYNILKDSKLHPLLRLR

SEQ ID NO: 49 - protein, 74 aa, synthetic construct
MLIFVKTLTGKTITLEVTPSDTIENVKAKIQDKEGIPPDQQRLAFAGKSLEDGRTLSDYNILKDSKLHPLLRLR

SEQ ID NO: 50 - protein, 74 aa, synthetic construct
MLIFVKTLTGKTITLEVNPSDTIENVKAKIQDKEGIPPDQQRLAFAGKSLEDGRTLSDYNILKDSKLHPLLRLR

SEQ ID NO: 51 - protein, 74 aa, synthetic construct
MLIFVKTLTGKTITLEVDPSDTIENVKAKIQDKEGIPPDQQRLAFAGKSLEDGRTLSDYNILKDSKLHPLLRLR

SEQ ID NO: 52 - protein, 74 aa, synthetic construct
MLIFVKTLTGKTITLEVCPSDTIENVKAKIQDKEGIPPDQQRLAFAGKSLEDGRTLSDYNILKDSKLHPLLRLR

SEQ ID NO: 53 - protein, 74 aa, synthetic construct
MLIFVKTLTGKTITLEVEKSDTIENVKAKIQDKEGIPPDQQRLAFAGKSLEDGRTLSDYNILKDSKLHPLLRLR

SEQ ID NO: 54 - protein, 74 aa, synthetic construct
MLIFVKTLTGKTITLEVEPADTIENVKAKIQDKEGIPPDQQRLAFAGKSLEDGRTLSDYNILKDSKLHPLLRLR

SEQ ID NO: 55 - protein, 74 aa, synthetic construct
MLIFVKTLTGKTITLEVEPNDTIENVKAKIQDKEGIPPDQQRLAFAGKSLEDGRTLSDYNILKDSKLHPLLRLR

SEQ ID NO: 56 - protein, 74 aa, synthetic construct
MLIFVKTLTGKTITLEVEPDDTIENVKAKIQDKEGIPPDQQRLAFAGKSLEDGRTLSDYNILKDSKLHPLLRLR

SEQ ID NO: 57 - protein, 74 aa, synthetic construct
MLIFVKTLTGKTITLEVEPCDTIENVKAKIQDKEGIPPDQQRLAFAGKSLEDGRTLSDYNILKDSKLHPLLRLR

SEQ ID NO: 58 - protein, 74 aa, synthetic construct
MLIFVKTLTGKTITLEVEPWDTIENVKAKIQDKEGIPPDQQRLAFAGKSLEDGRTLSDYNILKDSKLHPLLRLR

SEQ ID NO: 59 - protein, 74 aa, synthetic construct
MLIFVKTLTGKTITLEVEPSETIENVKAKIQDKEGIPPDQQRLAFAGKSLEDGRTLSDYNILKDSKLHPLLRLR

SEQ ID NO: 60 - protein, 74 aa, synthetic construct
MLIFVKTLTGKTITLEVEPSDTIECVKAKIQDKEGIPPDQQRLAFAGKSLEDGRTLSDYNILKDSKLHPLLRLR

SEQ ID NO: 61 - protein, 74 aa, synthetic construct
MLIFVKTLTGKTITLEVEPSDTIEGVKAKIQDKEGIPPDQQRLAFAGKSLEDGRTLSDYNILKDSKLHPLLRLR

SEQ ID NO: 62 - protein, 74 aa, synthetic construct
MLIFVKTLTGKTITLEVEPSDTIEIVKAKIQDKEGIPPDQQRLAFAGKSLEDGRTLSDYNILKDSKLHPLLRLR

SEQ ID NO: 63 - protein, 74 aa, synthetic construct
MLIFVKTLTGKTITLEVEPSDTIETVKAKIQDKEGIPPDQQRLAFAGKSLEDGRTLSDYNILKDSKLHPLLRLR

SEQ ID NO: 64 - protein, 74 aa, synthetic construct
MLIFVKTLTGKTITLEVEPSDTIEVVKAKIQDKEGIPPDQQRLAFAGKSLEDGRTLSDYNILKDSKLHPLLRLR

SEQ ID NO: 65 - protein, 74 aa, synthetic construct
MLIFVKTLTGKTITLEVEPSDTIEMVKAKIQDKEGIPPDQQRLAFAGKSLEDGRTLSDYNILKDSKLHPLLRLR

SEQ ID NO: 66 - protein, 74 aa, synthetic construct
MLIFVKTLTGKTITLEVEPSDTIELVKAKIQDKEGIPPDQQRLAFAGKSLEDGRTLSDYNILKDSKLHPLLRLR

SEQ ID NO: 67 - protein, 74 aa, synthetic construct
MLIFVKTLTGKTITLEVEPSDTIEFVKAKIQDKEGIPPDQQRLAFAGKSLEDGRTLSDYNILKDSKLHPLLRLR

SEQ ID NO: 68 - protein, 74 aa, synthetic construct
MLIFVKTLTGKTITLEVEPSDTIEEVKAKIQDKEGIPPDQQRLAFAGKSLEDGRTLSDYNILKDSKLHPLLRLR

SEQ ID NO: 69 - protein, 74 aa, synthetic construct
MLIFVKTLTGKTITLEVEPSDTIERVKAKIQDKEGIPPDQQRLAFAGKSLEDGRTLSDYNILKDSKLHPLLRLR

SEQ ID NO: 70 - protein, 74 aa, synthetic construct
MLIFVKTLTGKTITLEVEPSDTIEQVKAKIQDKEGIPPDQQRLAFAGKSLEDGRTLSDYNILKDSKLHPLLRLR

SEQ ID NO: 71 - protein, 74 aa, synthetic construct
MLIFVKTLTGKTITLEVEPSDTIESVKAKIQDKEGIPPDQQRLAFAGKSLEDGRTLSDYNILKDSKLHPLLRLR

SEQ ID NO: 72 - protein, 74 aa, synthetic construct
MLIFVKTLTGKTITLEVEPSDTIEAVKAKIQDKEGIPPDQQRLAFAGKSLEDGRTLSDYNILKDSKLHPLLRLR

SEQ ID NO: 73 - protein, 74 aa, synthetic construct
MLIFVKTLTGKTITLEVEPSDTIEDVKAKIQDKEGIPPDQQRLAFAGKSLEDGRTLSDYNILKDSKLHPLLRLR

SEQ ID NO: 74 - protein, 74 aa, synthetic construct
MLIFVKTLTGKTITLEVEPSDTIEKVKAKIQDKEGIPPDQQRLAFAGKSLEDGRTLSDYNILKDSKLHPLLRLR

SEQ ID NO: 75 - protein, 74 aa, synthetic construct
MLIFVKTLTGKTITLEVEPSDTIENIKAKIQDKEGIPPDQQRLAFAGKSLEDGRTLSDYNILKDSKLHPLLRLR

SEQ ID NO: 76 - protein, 74 aa, synthetic construct
MLIFVKTLTGKTITLEVEPSDTIENLKAKIQDKEGIPPDQQRLAFAGKSLEDGRTLSDYNILKDSKLHPLLRLR

SEQ ID NO: 77 - protein, 74 aa, synthetic construct
MLIFVKTLTGKTITLEVEPSDTIENVKDKIQDKEGIPPDQQRLAFAGKSLEDGRTLSDYNILKDSKLHPLLRLR

SEQ ID NO: 78 - protein, 74 aa, synthetic construct
MLIFVKTLTGKTITLEVEPSDTIENVKIKIQDKEGIPPDQQRLAFAGKSLEDGRTLSDYNILKDSKLHPLLRLR

SEQ ID NO: 79 - protein, 74 aa, synthetic construct
MLIFVKTLTGKTITLEVEPSDTIENVKMKIQDKEGIPPDQQRLAFAGKSLEDGRTLSDYNILKDSKLHPLLRLR

SEQ ID NO: 80 - protein, 74 aa, synthetic construct
MLIFVKTLTGKTITLEVEPSDTIENVKWKIQDKEGIPPDQQRLAFAGKSLEDGRTLSDYNILKDSKLHPLLRLR

SEQ ID NO: 81 - protein, 74 aa, synthetic construct
MLIFVKTLTGKTITLEVEPSDTIENVKQKIQDKEGIPPDQQRLAFAGKSLEDGRTLSDYNILKDSKLHPLLRLR

SEQ ID NO: 82 - protein, 74 aa, synthetic construct
MLIFVKTLTGKTITLEVEPSDTIENVKEKIQDKEGIPPDQQRLAFAGKSLEDGRTLSDYNILKDSKLHPLLRLR

SEQ ID NO: 83 - protein, 74 aa, synthetic construct
MLIFVKTLTGKTITLEVEPSDTIENVKAMIQDKEGIPPDQQRLAFAGKSLEDGRTLSDYNILKDSKLHPLLRLR

SEQ ID NO: 84 - protein, 74 aa, synthetic construct
MLIFVKTLTGKTITLEVEPSDTIENVKAHIQDKEGIPPDQQRLAFAGKSLEDGRTLSDYNILKDSKLHPLLRLR

SEQ ID NO: 85 - protein, 74 aa, synthetic construct
MLIFVKTLTGKTITLEVEPSDTIENVKALIQDKEGIPPDQQRLAFAGKSLEDGRTLSDYNILKDSKLHPLLRLR

SEQ ID NO: 86 - protein, 74 aa, synthetic construct
MLIFVKTLTGKTITLEVEPSDTIENVKARIQDKEGIPPDQQRLAFAGKSLEDGRTLSDYNILKDSKLHPLLRLR

SEQ ID NO: 87 - protein, 74 aa, synthetic construct
MLIFVKTLTGKTITLEVEPSDTIENVKAQIQDKEGIPPDQQRLAFAGKSLEDGRTLSDYNILKDSKLHPLLRLR

SEQ ID NO: 88 - protein, 74 aa, synthetic construct
MLIFVKTLTGKTITLEVEPSDTIENVKAKICDKEGIPPDQQRLAFAGKSLEDGRTLSDYNILKDSKLHPLLRLR

SEQ ID NO: 89 - protein, 74 aa, synthetic construct
MLIFVKTLTGKTITLEVEPSDTIENVKAKIWDKEGIPPDQQRLAFAGKSLEDGRTLSDYNILKDSKLHPLLRLR

SEQ ID NO: 90 - protein, 74 aa, synthetic construct
MLIFVKTLTGKTITLEVEPSDTIENVKAKIRDKEGIPPDQQRLAFAGKSLEDGRTLSDYNILKDSKLHPLLRLR

SEQ ID NO: 91 - protein, 74 aa, synthetic construct
MLIFVKTLTGKTITLEVEPSDTIENVKAKIHDKEGIPPDQQRLAFAGKSLEDGRTLSDYNILKDSKLHPLLRLR

SEQ ID NO: 92 - protein, 74 aa, synthetic construct
MLIFVKTLTGKTITLEVEPSDTIENVKAKIMDKEGIPPDQQRLAFAGKSLEDGRTLSDYNILKDSKLHPLLRLR

SEQ ID NO: 93 - protein, 74 aa, synthetic construct
MLIFVKTLTGKTITLEVEPSDTIENVKAKIFDKEGIPPDQQRLAFAGKSLEDGRTLSDYNILKDSKLHPLLRLR

SEQ ID NO: 94 - protein, 74 aa, synthetic construct
MLIFVKTLTGKTITLEVEPSDTIENVKAKILDKEGIPPDQQRLAFAGKSLEDGRTLSDYNILKDSKLHPLLRLR

SEQ ID NO: 95 - protein, 74 aa, synthetic construct
MLIFVKTLTGKTITLEVEPSDTIENVKAKIYDKEGIPPDQQRLAFAGKSLEDGRTLSDYNILKDSKLHPLLRLR

SEQ ID NO: 96 - protein, 74 aa, synthetic construct
MLIFVKTLTGKTITLEVEPSDTIENVKAKIQRKEGIPPDQQRLAFAGKSLEDGRTLSDYNILKDSKLHPLLRLR

SEQ ID NO: 97 - protein, 74 aa, synthetic construct
MLIFVKTLTGKTITLEVEPSDTIENVKAKIQEKEGIPPDQQRLAFAGKSLEDGRTLSDYNILKDSKLHPLLRLR

SEQ ID NO: 98 - protein, 74 aa, synthetic construct
MLIFVKTLTGKTITLEVEPSDTIENVKAKIQAKEGIPPDQQRLAFAGKSLEDGRTLSDYNILKDSKLHPLLRLR

SEQ ID NO: 99 - protein, 74 aa, synthetic construct
MLIFVKTLTGKTITLEVEPSDTIENVKAKIQDHEGIPPDQQRLAFAGKSLEDGRTLSDYNILKDSKLHPLLRLR

SEQ ID NO: 100 - protein, 74 aa, synthetic construct
MLIFVKTLTGKTITLEVEPSDTIENVKAKIQDAEGIPPDQQRLAFAGKSLEDGRTLSDYNILKDSKLHPLLRLR

SEQ ID NO: 101 - protein, 74 aa, synthetic construct
MLIFVKTLTGKTITLEVEPSDTIENVKAKIQDCEGIPPDQQRLAFAGKSLEDGRTLSDYNILKDSKLHPLLRLR

SEQ ID NO: 102 - protein, 74 aa, synthetic construct
MLIFVKTLTGKTITLEVEPSDTIENVKAKIQDEEGIPPDQQRLAFAGKSLEDGRTLSDYNILKDSKLHPLLRLR

SEQ ID NO: 103 - protein, 74 aa, synthetic construct
MLIFVKTLTGKTITLEVEPSDTIENVKAKIQDIEGIPPDQQRLAFAGKSLEDGRTLSDYNILKDSKLHPLLRLR

SEQ ID NO: 104 - protein, 74 aa, synthetic construct
MLIFVKTLTGKTITLEVEPSDTIENVKAKIQDQEGIPPDQQRLAFAGKSLEDGRTLSDYNILKDSKLHPLLRLR

SEQ ID NO: 105 - protein, 74 aa, synthetic construct
MLIFVKTLTGKTITLEVEPSDTIENVKAKIQDSEGIPPDQQRLAFAGKSLEDGRTLSDYNILKDSKLHPLLRLR

SEQ ID NO: 106 - protein, 74 aa, synthetic construct
MLIFVKTLTGKTITLEVEPSDTIENVKAKIQDVEGIPPDQQRLAFAGKSLEDGRTLSDYNILKDSKLHPLLRLR

SEQ ID NO: 107 - protein, 74 aa, synthetic construct
MLIFVKTLTGKTITLEVEPSDTIENVKAKIQDLEGIPPDQQRLAFAGKSLEDGRTLSDYNILKDSKLHPLLRLR

SEQ ID NO: 108 - protein, 74 aa, synthetic construct
MLIFVKTLTGKTITLEVEPSDTIENVKAKIQDMEGIPPDQQRLAFAGKSLEDGRTLSDYNILKDSKLHPLLRLR

SEQ ID NO: 109 - protein, 74 aa, synthetic construct
MLIFVKTLTGKTITLEVEPSDTIENVKAKIQDTEGIPPDQQRLAFAGKSLEDGRTLSDYNILKDSKLHPLLRLR

SEQ ID NO: 110 - protein, 74 aa, synthetic construct
MLIFVKTLTGKTITLEVEPSDTIENVKAKIQDREGIPPDQQRLAFAGKSLEDGRTLSDYNILKDSKLHPLLRLR

SEQ ID NO: 111 - protein, 74 aa, synthetic construct
MLIFVKTLTGKTITLEVEPSDTIENVKAKIQDFEGIPPDQQRLAFAGKSLEDGRTLSDYNILKDSKLHPLLRLR

SEQ ID NO: 112 - protein, 74 aa, synthetic construct
MLIFVKTLTGKTITLEVEPSDTIENVKAKIQDYEGIPPDQQRLAFAGKSLEDGRTLSDYNILKDSKLHPLLRLR

SEQ ID NO: 113 - protein, 74 aa, synthetic construct
MLIFVKTLTGKTITLEVEPSDTIENVKAKIQDNEGIPPDQQRLAFAGKSLEDGRTLSDYNILKDSKLHPLLRLR

SEQ ID NO: 114 - protein, 74 aa, synthetic construct
MLIFVKTLTGKTITLEVEPSDTIENVKAKIQDWEGIPPDQQRLAFAGKSLEDGRTLSDYNILKDSKLHPLLRLR

SEQ ID NO: 115 - protein, 74 aa, synthetic construct
MLIFVKTLTGKTITLEVEPSDTIENVKAKIQDKTGIPPDQQRLAFAGKSLEDGRTLSDYNILKDSKLHPLLRLR

SEQ ID NO: 116 - protein, 74 aa, synthetic construct
MLIFVKTLTGKTITLEVEPSDTIENVKAKIQDKEGIPLDQQRLAFAGKSLEDGRTLSDYNILKDSKLHPLLRLR

SEQ ID NO: 117 - protein, 74 aa, synthetic construct
MLIFVKTLTGKTITLEVEPSDTIENVKAKIQDKEGIPVDQQRLAFAGKSLEDGRTLSDYNILKDSKLHPLLRLR

SEQ ID NO: 118 - protein, 74 aa, synthetic construct
MLIFVKTLTGKTITLEVEPSDTIENVKAKIQDKEGIPSDQQRLAFAGKSLEDGRTLSDYNILKDSKLHPLLRLR

SEQ ID NO: 119 - protein, 74 aa, synthetic construct
MLIFVKTLTGKTITLEVEPSDTIENVKAKIQDKEGIPTDQQRLAFAGKSLEDGRTLSDYNILKDSKLHPLLRLR

SEQ ID NO: 120 - protein, 74 aa, synthetic construct
MLIFVKTLTGKTITLEVEPSDTIENVKAKIQDKEGIPCDQQRLAFAGKSLEDGRTLSDYNILKDSKLHPLLRLR

SEQ ID NO: 121 - protein, 74 aa, synthetic construct
MLIFVKTLTGKTITLEVEPSDTIENVKAKIQDKEGIPFDQQRLAFAGKSLEDGRTLSDYNILKDSKLHPLLRLR

SEQ ID NO: 122 - protein, 74 aa, synthetic construct
MLIFVKTLTGKTITLEVEPSDTIENVKAKIQDKEGIPWDQQRLAFAGKSLEDGRTLSDYNILKDSKLHPLLRLR

SEQ ID NO: 123 - protein, 74 aa, synthetic construct
MLIFVKTLTGKTITLEVEPSDTIENVKAKIQDKEGIPIDQQRLAFAGKSLEDGRTLSDYNILKDSKLHPLLRLR

SEQ ID NO: 124 - protein, 74 aa, synthetic construct
MLIFVKTLTGKTITLEVEPSDTIENVKAKIQDKEGIPADQQRLAFAGKSLEDGRTLSDYNILKDSKLHPLLRLR

SEQ ID NO: 125 - protein, 74 aa, synthetic construct
MLIFVKTLTGKTITLEVEPSDTIENVKAKIQDKEGIPNDQQRLAFAGKSLEDGRTLSDYNILKDSKLHPLLRLR

SEQ ID NO: 126 - protein, 74 aa, synthetic construct
MLIFVKTLTGKTITLEVEPSDTIENVKAKIQDKEGIPQDQQRLAFAGKSLEDGRTLSDYNILKDSKLHPLLRLR

SEQ ID NO: 127 - protein, 74 aa, synthetic construct
MLIFVKTLTGKTITLEVEPSDTIENVKAKIQDKEGIPHDQQRLAFAGKSLEDGRTLSDYNILKDSKLHPLLRLR

SEQ ID NO: 128 - protein, 74 aa, synthetic construct
MLIFVKTLTGKTITLEVEPSDTIENVKAKIQDKEGIPKDQQRLAFAGKSLEDGRTLSDYNILKDSKLHPLLRLR

SEQ ID NO: 129 - protein, 74 aa, synthetic construct
MLIFVKTLTGKTITLEVEPSDTIENVKAKIQDKEGIPMDQQRLAFAGKSLEDGRTLSDYNILKDSKLHPLLRLR

SEQ ID NO: 130 - protein, 74 aa, synthetic construct
MLIFVKTLTGKTITLEVEPSDTIENVKAKIQDKEGIPYDQQRLAFAGKSLEDGRTLSDYNILKDSKLHPLLRLR

SEQ ID NO: 131 - protein, 74 aa, synthetic construct
MLIFVKTLTGKTITLEVEPSDTIENVKAKIQDKEGIPPQQQRLAFAGKSLEDGRTLSDYNILKDSKLHPLLRLR

SEQ ID NO: 132 - protein, 74 aa, synthetic construct
MLIFVKTLTGKTITLEVEPSDTIENVKAKIQDKEGIPPGQQRLAFAGKSLEDGRTLSDYNILKDSKLHPLLRLR

SEQ ID NO: 133 - protein, 74 aa, synthetic construct
MLIFVKTLTGKTITLEVEPSDTIENVKAKIQDKEGIPPLQQRLAFAGKSLEDGRTLSDYNILKDSKLHPLLRLR

SEQ ID NO: 134 - protein, 74 aa, synthetic construct
MLIFVKTLTGKTITLEVEPSDTIENVKAKIQDKEGIPPSQQRLAFAGKSLEDGRTLSDYNILKDSKLHPLLRLR

SEQ ID NO: 135 - protein, 74 aa, synthetic construct
MLIFVKTLTGKTITLEVEPSDTIENVKAKIQDKEGIPPWQQRLAFAGKSLEDGRTLSDYNILKDSKLHPLLRLR

SEQ ID NO: 136 - protein, 74 aa, synthetic construct
MLIFVKTLTGKTITLEVEPSDTIENVKAKIQDKEGIPPEQQRLAFAGKSLEDGRTLSDYNILKDSKLHPLLRLR

SEQ ID NO: 137 - protein, 74 aa, synthetic construct
MLIFVKTLTGKTITLEVEPSDTIENVKAKIQDKEGIPPDDQRLAFAGKSLEDGRTLSDYNILKDSKLHPLLRLR

SEQ ID NO: 138 - protein, 74 aa, synthetic construct
MLIFVKTLTGKTITLEVEPSDTIENVKAKIQDKEGIPPDEQRLAFAGKSLEDGRTLSDYNILKDSKLHPLLRLR

SEQ ID NO: 139 - protein, 74 aa, synthetic construct
MLIFVKTLTGKTITLEVEPSDTIENVKAKIQDKEGIPPDQVRLAFAGKSLEDGRTLSDYNILKDSKLHPLLRLR

SEQ ID NO: 140 - protein, 74 aa, synthetic construct
MLIFVKTLTGKTITLEVEPSDTIENVKAKIQDKEGIPPDQYRLAFAGKSLEDGRTLSDYNILKDSKLHPLLRLR

SEQ ID NO: 141 - protein, 74 aa, synthetic construct
MLIFVKTLTGKTITLEVEPSDTIENVKAKIQDKEGIPPDQIRLAFAGKSLEDGRTLSDYNILKDSKLHPLLRLR

SEQ ID NO: 142 - protein, 74 aa, synthetic construct
MLIFVKTLTGKTITLEVEPSDTIENVKAKIQDKEGIPPDQCRLAFAGKSLEDGRTLSDYNILKDSKLHPLLRLR

SEQ ID NO: 143 - protein, 74 aa, synthetic construct
MLIFVKTLTGKTITLEVEPSDTIENVKAKIQDKEGIPPDQQSLAFAGKSLEDGRTLSDYNILKDSKLHPLLRLR

SEQ ID NO: 144 - protein, 74 aa, synthetic construct
MLIFVKTLTGKTITLEVEPSDTIENVKAKIQDKEGIPPDQQHLAFAGKSLEDGRTLSDYNILKDSKLHPLLRLR

SEQ ID NO: 145 - protein, 74 aa, synthetic construct
MLIFVKTLTGKTITLEVEPSDTIENVKAKIQDKEGIPPDQQFLAFAGKSLEDGRTLSDYNILKDSKLHPLLRLR

SEQ ID NO: 146 - protein, 74 aa, synthetic construct
MLIFVKTLTGKTITLEVEPSDTIENVKAKIQDKEGIPPDQQWLAFAGKSLEDGRTLSDYNILKDSKLHPLLRLR

SEQ ID NO: 147 - protein, 74 aa, synthetic construct
MLIFVKTLTGKTITLEVEPSDTIENVKAKIQDKEGIPPDQQYLAFAGKSLEDGRTLSDYNILKDSKLHPLLRLR

SEQ ID NO: 148 - protein, 74 aa, synthetic construct
MLIFVKTLTGKTITLEVEPSDTIENVKAKIQDKEGIPPDQQNLAFAGKSLEDGRTLSDYNILKDSKLHPLLRLR

SEQ ID NO: 149 - protein, 74 aa, synthetic construct
MLIFVKTLTGKTITLEVEPSDTIENVKAKIQDKEGIPPDQQCLAFAGKSLEDGRTLSDYNILKDSKLHPLLRLR

SEQ ID NO: 150 - protein, 74 aa, synthetic construct
MLIFVKTLTGKTITLEVEPSDTIENVKAKIQDKEGIPPDQQRLTFAGKSLEDGRTLSDYNILKDSKLHPLLRLR

SEQ ID NO: 151 - protein, 74 aa, synthetic construct
MLIFVKTLTGKTITLEVEPSDTIENVKAKIQDKEGIPPDQQRLAFQGKSLEDGRTLSDYNILKDSKLHPLLRLR

SEQ ID NO: 152 - protein, 74 aa, synthetic construct
MLIFVKTLTGKTITLEVEPSDTIENVKAKIQDKEGIPPDQQRLAFGGKSLEDGRTLSDYNILKDSKLHPLLRLR

SEQ ID NO: 153 - protein, 74 aa, synthetic construct
MLIFVKTLTGKTITLEVEPSDTIENVKAKIQDKEGIPPDQQRLAFAGNSLEDGRTLSDYNILKDSKLHPLLRLR

SEQ ID NO: 154 - protein, 74 aa, synthetic construct
MLIFVKTLTGKTITLEVEPSDTIENVKAKIQDKEGIPPDQQRLAFAGTSLEDGRTLSDYNILKDSKLHPLLRLR

SEQ ID NO: 155 - protein, 74 aa, synthetic construct
MLIFVKTLTGKTITLEVEPSDTIENVKAKIQDKEGIPPDQQRLAFAGMSLEDGRTLSDYNILKDSKLHPLLRLR

SEQ ID NO: 156 - protein, 74 aa, synthetic construct
MLIFVKTLTGKTITLEVEPSDTIENVKAKIQDKEGIPPDQQRLAFAGVSLEDGRTLSDYNILKDSKLHPLLRLR

SEQ ID NO: 157 - protein, 74 aa, synthetic construct
MLIFVKTLTGKTITLEVEPSDTIENVKAKIQDKEGIPPDQQRLAFAGQSLEDGRTLSDYNILKDSKLHPLLRLR

SEQ ID NO: 158 - protein, 74 aa, synthetic construct
MLIFVKTLTGKTITLEVEPSDTIENVKAKIQDKEGIPPDQQRLAFAGISLEDGRTLSDYNILKDSKLHPLLRLR

SEQ ID NO: 159 - protein, 74 aa, synthetic construct
MLIFVKTLTGKTITLEVEPSDTIENVKAKIQDKEGIPPDQQRLAFAGRSLEDGRTLSDYNILKDSKLHPLLRLR

SEQ ID NO: 160 - protein, 74 aa, synthetic construct
MLIFVKTLTGKTITLEVEPSDTIENVKAKIQDKEGIPPDQQRLAFAGLSLEDGRTLSDYNILKDSKLHPLLRLR

SEQ ID NO: 161 - protein, 74 aa, synthetic construct
MLIFVKTLTGKTITLEVEPSDTIENVKAKIQDKEGIPPDQQRLAFAGKMLEDGRTLSDYNILKDSKLHPLLRLR

SEQ ID NO: 162 - protein, 74 aa, synthetic construct
MLIFVKTLTGKTITLEVEPSDTIENVKAKIQDKEGIPPDQQRLAFAGKCLEDGRTLSDYNILKDSKLHPLLRLR

SEQ ID NO: 163 - protein, 74 aa, synthetic construct
MLIFVKTLTGKTITLEVEPSDTIENVKAKIQDKEGIPPDQQRLAFAGKLLEDGRTLSDYNILKDSKLHPLLRLR

SEQ ID NO: 164 - protein, 74 aa, synthetic construct
MLIFVKTLTGKTITLEVEPSDTIENVKAKIQDKEGIPPDQQRLAFAGKVLEDGRTLSDYNILKDSKLHPLLRLR

SEQ ID NO: 165 - protein, 74 aa, synthetic construct
MLIFVKTLTGKTITLEVEPSDTIENVKAKIQDKEGIPPDQQRLAFAGKPLEDGRTLSDYNILKDSKLHPLLRLR

SEQ ID NO: 166 - protein, 74 aa, synthetic construct
MLIFVKTLTGKTITLEVEPSDTIENVKAKIQDKEGIPPDQQRLAFAGKALEDGRTLSDYNILKDSKLHPLLRLR

SEQ ID NO: 167 - protein, 74 aa, synthetic construct
MLIFVKTLTGKTITLEVEPSDTIENVKAKIQDKEGIPPDQQRLAFAGKILEDGRTLSDYNILKDSKLHPLLRLR

SEQ ID NO: 168 - protein, 74 aa, synthetic construct
MLIFVKTLTGKTITLEVEPSDTIENVKAKIQDKEGIPPDQQRLAFAGKNLEDGRTLSDYNILKDSKLHPLLRLR

SEQ ID NO: 169 - protein, 74 aa, synthetic construct
MLIFVKTLTGKTITLEVEPSDTIENVKAKIQDKEGIPPDQQRLAFAGKGLEDGRTLSDYNILKDSKLHPLLRLR

SEQ ID NO: 170 - protein, 74 aa, synthetic construct
MLIFVKTLTGKTITLEVEPSDTIENVKAKIQDKEGIPPDQQRLAFAGKELEDGRTLSDYNILKDSKLHPLLRLR

SEQ ID NO: 171 - protein, 74 aa, synthetic construct
MLIFVKTLTGKTITLEVEPSDTIENVKAKIQDKEGIPPDQQRLAFAGKDLEDGRTLSDYNILKDSKLHPLLRLR

SEQ ID NO: 172 - protein, 74 aa, synthetic construct
MLIFVKTLTGKTITLEVEPSDTIENVKAKIQDKEGIPPDQQRLAFAGKSLDDGRTLSDYNILKDSKLHPLLRLR

SEQ ID NO: 173 - protein, 74 aa, synthetic construct
MLIFVKTLTGKTITLEVEPSDTIENVKAKIQDKEGIPPDQQRLAFAGKSLEEGRTLSDYNILKDSKLHPLLRLR

SEQ ID NO: 174 - protein, 74 aa, synthetic construct
MLIFVKTLTGKTITLEVEPSDTIENVKAKIQDKEGIPPDQQRLAFAGKSLEDGNTLSDYNILKDSKLHPLLRLR

SEQ ID NO: 175 - protein, 74 aa, synthetic construct
MLIFVKTLTGKTITLEVEPSDTIENVKAKIQDKEGIPPDQQRLAFAGKSLEDGCTLSDYNILKDSKLHPLLRLR

SEQ ID NO: 176 - protein, 74 aa, synthetic construct
MLIFVKTLTGKTITLEVEPSDTIENVKAKIQDKEGIPPDQQRLAFAGKSLEDGQTLSDYNILKDSKLHPLLRLR

SEQ ID NO: 177 - protein, 74 aa, synthetic construct
MLIFVKTLTGKTITLEVEPSDTIENVKAKIQDKEGIPPDQQRLAFAGKSLEDGFTLSDYNILKDSKLHPLLRLR

SEQ ID NO: 178 - protein, 74 aa, synthetic construct
MLIFVKTLTGKTITLEVEPSDTIENVKAKIQDKEGIPPDQQRLAFAGKSLEDGYTLSDYNILKDSKLHPLLRLR

SEQ ID NO: 179 - protein, 74 aa, synthetic construct
MLIFVKTLTGKTITLEVEPSDTIENVKAKIQDKEGIPPDQQRLAFAGKSLEDGMTLSDYNILKDSKLHPLLRLR

SEQ ID NO: 180 - protein, 74 aa, synthetic construct
MLIFVKTLTGKTITLEVEPSDTIENVKAKIQDKEGIPPDQQRLAFAGKSLEDGHTLSDYNILKDSKLHPLLRLR

SEQ ID NO: 181 - protein, 74 aa, synthetic construct
MLIFVKTLTGKTITLEVEPSDTIENVKAKIQDKEGIPPDQQRLAFAGKSLEDGTTLSDYNILKDSKLHPLLRLR

SEQ ID NO: 182 - protein, 74 aa, synthetic construct
MLIFVKTLTGKTITLEVEPSDTIENVKAKIQDKEGIPPDQQRLAFAGKSLEDGKTLSDYNILKDSKLHPLLRLR

SEQ ID NO: 183 - protein, 74 aa, synthetic construct
MLIFVKTLTGKTITLEVEPSDTIENVKAKIQDKEGIPPDQQRLAFAGKSLEDGRRLSDYNILKDSKLHPLLRLR

SEQ ID NO: 184 - protein, 74 aa, synthetic construct
MLIFVKTLTGKTITLEVEPSDTIENVKAKIQDKEGIPPDQQRLAFAGKSLEDGRTLNDYNILKDSKLHPLLRLR

SEQ ID NO: 185 - protein, 74 aa, synthetic construct
MLIFVKTLTGKTITLEVEPSDTIENVKAKIQDKEGIPPDQQRLAFAGKSLEDGRTLGDYNILKDSKLHPLLRLR

SEQ ID NO: 186 - protein, 74 aa, synthetic construct
MLIFVKTLTGKTITLEVEPSDTIENVKAKIQDKEGIPPDQQRLAFAGKSLEDGRTLDDYNILKDSKLHPLLRLR

SEQ ID NO: 187 - protein, 74 aa, synthetic construct
MLIFVKTLTGKTITLEVEPSDTIENVKAKIQDKEGIPPDQQRLAFAGKSLEDGRTLHDYNILKDSKLHPLLRLR

SEQ ID NO: 188 - protein, 74 aa, synthetic construct
MLIFVKTLTGKTITLEVEPSDTIENVKAKIQDKEGIPPDQQRLAFAGKSLEDGRTLADYNILKDSKLHPLLRLR

SEQ ID NO: 189 - protein, 74 aa, synthetic construct
MLIFVKTLTGKTITLEVEPSDTIENVKAKIQDKEGIPPDQQRLAFAGKSLEDGRTLEDYNILKDSKLHPLLRLR

SEQ ID NO: 190 - protein, 74 aa, synthetic construct
MLIFVKTLTGKTITLEVEPSDTIENVKAKIQDKEGIPPDQQRLAFAGKSLEDGRTLQDYNILKDSKLHPLLRLR

SEQ ID NO: 191 - protein, 74 aa, synthetic construct
MLIFVKTLTGKTITLEVEPSDTIENVKAKIQDKEGIPPDQQRLAFAGKSLEDGRTLRDYNILKDSKLHPLLRLR

SEQ ID NO: 192 - protein, 74 aa, synthetic construct
MLIFVKTLTGKTITLEVEPSDTIENVKAKIQDKEGIPPDQQRLAFAGKSLEDGRTLKDYNILKDSKLHPLLRLR

SEQ ID NO: 193 - protein, 74 aa, synthetic construct
MLIFVKTLTGKTITLEVEPSDTIENVKAKIQDKEGIPPDQQRLAFAGKSLEDGRTLMDYNILKDSKLHPLLRLR

SEQ ID NO: 194 - protein, 74 aa, synthetic construct
MLIFVKTLTGKTITLEVEPSDTIENVKAKIQDKEGIPPDQQRLAFAGKSLEDGRTLSSYNILKDSKLHPLLRLR

SEQ ID NO: 195 - protein, 74 aa, synthetic construct
MLIFVKTLTGKTITLEVEPSDTIENVKAKIQDKEGIPPDQQRLAFAGKSLEDGRTLSDYEILKDSKLHPLLRLR

SEQ ID NO: 196 - protein, 74 aa, synthetic construct
MLIFVKTLTGKTITLEVEPSDTIENVKAKIQDKEGIPPDQQRLAFAGKSLEDGRTLSDYQILKDSKLHPLLRLR

SEQ ID NO: 197 - protein, 74 aa, synthetic construct
MLIFVKTLTGKTITLEVEPSDTIENVKAKIQDKEGIPPDQQRLAFAGKSLEDGRTLSDYNLLKDSKLHPLLRLR

SEQ ID NO: 198 - protein, 74 aa, synthetic construct
MLIFVKTLTGKTITLEVEPSDTIENVKAKIQDKEGIPPDQQRLAFAGKSLEDGRTLSDYNILMDSKLHPLLRLR

SEQ ID NO: 199 - protein, 74 aa, synthetic construct
MLIFVKTLTGKTITLEVEPSDTIENVKAKIQDKEGIPPDQQRLAFAGKSLEDGRTLSDYNILFDSKLHPLLRLR

SEQ ID NO: 200 - protein, 74 aa, synthetic construct
MLIFVKTLTGKTITLEVEPSDTIENVKAKIQDKEGIPPDQQRLAFAGKSLEDGRTLSDYNILVDSKLHPLLRLR

SEQ ID NO: 201 - protein, 74 aa, synthetic construct
MLIFVKTLTGKTITLEVEPSDTIENVKAKIQDKEGIPPDQQRLAFAGKSLEDGRTLSDYNILIDSKLHPLLRLR

SEQ ID NO: 202 - protein, 74 aa, synthetic construct
MLIFVKTLTGKTITLEVEPSDTIENVKAKIQDKEGIPPDQQRLAFAGKSLEDGRTLSDYNILKDPKLHPLLRLR

SEQ ID NO: 203 - protein, 74 aa, synthetic construct
MLIFVKTLTGKTITLEVEPSDTIENVKAKIQDKEGIPPDQQRLAFAGKSLEDGRTLSDYNILKDKKLHPLLRLR

SEQ ID NO: 204 - protein, 74 aa, synthetic construct
MLIFVKTLTGKTITLEVEPSDTIENVKAKIQDKEGIPPDQQRLAFAGKSLEDGRTLSDYNILKDAKLHPLLRLR

SEQ ID NO: 205 - protein, 74 aa, synthetic construct
MLIFVKTLTGKTITLEVEPSDTIENVKAKIQDKEGIPPDQQRLAFAGKSLEDGRTLSDYNILKDEKLHPLLRLR

SEQ ID NO: 206 - protein, 74 aa, synthetic construct
MLIFVKTLTGKTITLEVEPSDTIENVKAKIQDKEGIPPDQQRLAFAGKSLEDGRTLSDYNILKDRKLHPLLRLR

SEQ ID NO: 207 - protein, 74 aa, synthetic construct
MLIFVKTLTGKTITLEVEPSDTIENVKAKIQDKEGIPPDQQRLAFAGKSLEDGRTLSDYNILKDQKLHPLLRLR

SEQ ID NO: 208 - protein, 74 aa, synthetic construct
MLIFVKTLTGKTITLEVEPSDTIENVKAKIQDKEGIPPDQQRLAFAGKSLEDGRTLSDYNILKDHKLHPLLRLR

SEQ ID NO: 209 - protein, 74 aa, synthetic construct
MLIFVKTLTGKTITLEVEPSDTIENVKAKIQDKEGIPPDQQRLAFAGKSLEDGRTLSDYNILKDNKLHPLLRLR

SEQ ID NO: 210 - protein, 74 aa, synthetic construct
MLIFVKTLTGKTITLEVEPSDTIENVKAKIQDKEGIPPDQQRLAFAGKSLEDGRTLSDYNILKDDKLHPLLRLR

SEQ ID NO: 211 - protein, 74 aa, synthetic construct
MLIFVKTLTGKTITLEVEPSDTIENVKAKIQDKEGIPPDQQRLAFAGKSLEDGRTLSDYNILKDSRLHPLLRLR

SEQ ID NO: 212 - protein, 74 aa, synthetic construct
MLIFVKTLTGKTITLEVEPSDTIENVKAKIQDKEGIPPDQQRLAFAGKSLEDGRTLSDYNILKDSKCHPLLRLR

SEQ ID NO: 213 - protein, 74 aa, synthetic construct
MLIFVKTLTGKTITLEVEPSDTIENVKAKIQDKEGIPPDQQRLAFAGKSLEDGRTLSDYNILKDSKYHPLLRLR

SEQ ID NO: 214 - protein, 74 aa, synthetic construct
MLIFVKTLTGKTITLEVEPSDTIENVKAKIQDKEGIPPDQQRLAFAGKSLEDGRTLSDYNILKDSKHHPLLRLR

SEQ ID NO: 215 - protein, 74 aa, synthetic construct
MLIFVKTLTGKTITLEVEPSDTIENVKAKIQDKEGIPPDQQRLAFAGKSLEDGRTLSDYNILKDSKTHPLLRLR

SEQ ID NO: 216 - protein, 74 aa, synthetic construct
MLIFVKTLTGKTITLEVEPSDTIENVKAKIQDKEGIPPDQQRLAFAGKSLEDGRTLSDYNILKDSKKHPLLRLR

SEQ ID NO: 217 - protein, 74 aa, synthetic construct
MLIFVKTLTGKTITLEVEPSDTIENVKAKIQDKEGIPPDQQRLAFAGKSLEDGRTLSDYNILKDSKRHPLLRLR

SEQ ID NO: 218 - protein, 74 aa, synthetic construct
MLIFVKTLTGKTITLEVEPSDTIENVKAKIQDKEGIPPDQQRLAFAGKSLEDGRTLSDYNILKDSKSHPLLRLR

SEQ ID NO: 219 - protein, 74 aa, synthetic construct
MLIFVKTLTGKTITLEVEPSDTIENVKAKIQDKEGIPPDQQRLAFAGKSLEDGRTLSDYNILKDSKMHPLLRLR

SEQ ID NO: 220 - protein, 74 aa, synthetic construct
MLIFVKTLTGKTITLEVEPSDTIENVKAKIQDKEGIPPDQQRLAFAGKSLEDGRTLSDYNILKDSKLEPLLRLR

SEQ ID NO: 221 - protein, 74 aa, synthetic construct
MLIFVKTLTGKTITLEVEPSDTIENVKAKIQDKEGIPPDQQRLAFAGKSLEDGRTLSDYNILKDSKLMPLLRLR

SEQ ID NO: 222 - protein, 74 aa, synthetic construct
MLIFVKTLTGKTITLEVEPSDTIENVKAKIQDKEGIPPDQQRLAFAGKSLEDGRTLSDYNILKDSKLQPLLRLR

SEQ ID NO: 223 - protein, 74 aa, synthetic construct
MLIFVKTLTGKTITLEVEPSDTIENVKAKIQDKEGIPPDQQRLAFAGKSLEDGRTLSDYNILKDSKLHRLLRLR

SEQ ID NO: 224 - protein, 74 aa, synthetic construct
MLIFVKTLTGKTITLEVEPSDTIENVKAKIQDKEGIPPDQQRLAFAGKSLEDGRTLSDYNILKDSKLHPLLRMR

SEQ ID NO: 225 - protein, 74 aa, synthetic construct
MLIFVKTLTGKTITLEVEPSDTIENVKAKIQDKEGIPPDQQRLAFAGKSLEDGRTLSDYNILKDSKLHPLLRLQ

SEQ ID NO: 226 - protein, 74 aa, synthetic construct
MLIFVKTLTGKTITLEVEPSDTIENVKAKIQDKEGIPPDQQRLAFAGKSLEDGRTLSDYNILKDSKLHPLLRLV

SEQ ID NO: 227 - protein, 74 aa, synthetic construct
MLIFVKTLTGKTITLEVEPSDTIENVKAKIQDKEGIPPDQQRLAFAGKSLEDGRTLSDYNILKDSKLHPLLRLL

SEQ ID NO: 228 - protein, 74 aa, synthetic construct
MLIFVKTLTGKTITLEVEPSDTIENVKAKIQDKEGIPPDQQRLAFAGKSLEDGRTLSDYNILKDSKLHPLLRLM

SEQ ID NO: 229 - protein, 74 aa, synthetic construct
MLIFVKTLTGKTITLEVEPSDTIENVKAKIQDKEGIPPDQQRLAFAGKSLEDGRTLSDYNILKDSKLHPLLRLI

SEQ ID NO: 230 - protein, 74 aa, synthetic construct
MLIFVKTLTGKTITLEVEPSDTIENVKAKIQDKEGIPPDQQRLAFAGKSLEDGRTLSDYNILKDSKLHPLLRLC

SEQ ID NO: 231 - protein, 74 aa, synthetic construct
MLIFVKTLTGKTITLEVEPSDTIENVKAKIQDKEGIPPDQQRLAFAGKSLEDGRTLSDYNILKDSKLHPLLRLE

SEQ ID NO: 232 - protein, 74 aa, synthetic construct
MLIFVKTLTGKTITLEVEPSDTIENVKAKIQDKEGIPPDQQRLAFAGKSLEDGRTLSDYNILKDSKLHPLLRLT

SEQ ID NO: 233 - protein, 74 aa, synthetic construct
MLIFVKTLTGKTITLEVEPSDTIENVKAKIQDKEGIPPDQQRLAFAGKSLEDGRTLSDYNILKDSKLHPLLRLK

SEQ ID NO: 234 - protein, 85 aa, synthetic construct
MHHHHHHGGSGMLIFVKTLTGKTITLEVEPSDTIENVKAKIQDKEGIPPDQQRLAFAGKSLEDGRTLSDYNILKDSKLHLVLRLR

SEQ ID NO: 235 - protein, 85 aa, synthetic construct
MHHHHHHGGSGMLIFVRTLTGKTITLEVEPSDTIENVKAKIQDKEGIPPDQQRLAFAGKSLEDGRTLSDYNILKDSKLHPLLRLR

SEQ ID NO: 236 - protein, 85 aa, synthetic construct
MHHHHHHGGSGMLIFVKTLTGKTIELEVEPSDTIENVKAKIQDKEGIPPDQQRLAFAGKSLEDGRTLSDYNILKDSKLHPLLRLR

SEQ ID NO: 237 - protein, 85 aa, synthetic construct
MHHHHHHGGSGMLIFVKTLTGKTITLEVEPSDTIENVKAKIQDAEGIPPDQQRLAFAGKSLEDGRTLSDYNILKDSKLHPLLRLR

SEQ ID NO: 238 - protein, 85 aa, synthetic construct
MHHHHHHGGSGMLIFVKTLTGKTITLEVEPSDTIENVKAKIQDKEGIPPDQQRLAFQGKSLEDGRTLSDYNILKDSKLHPLLRLR

SEQ ID NO: 239 - protein, 85 aa, synthetic construct
MHHHHHHGGSGMLIFVKTLTGKTITLEVEPSDTIENVKAKIQDKEGIPPDQQRLAFAGKSLEDGRTLSDYNILIDSKLHPLLRLR

SEQ ID NO: 240 - protein, 85 aa, synthetic construct
MHHHHHHGGSGMLIFVKTLTGKTITLEVEPSDTIENVKAKIQDKEGIPPDQQRLAFAGKSLEDGRTLSDYNILKDPKLHPLLRLR

SEQ ID NO: 241 - protein, 85 aa, synthetic construct
MHHHHHHGGSGMLIFVRMLTGKMIELEVEPSDTIENVKAKIQDHEGIPPDQQRLAFQGKSLEDGRTLSDYNILKDPKKMPLLRLR

SEQ ID NO: 242 - protein, 85 aa, synthetic construct
MHHHHHHGGSGMLIFVRTLTGKTITLEVEPSDTIENVKAKIQDHEGIPPDQQRLAFQGKSLEDGRTLSDYNILKDPKLHPLLRLR

SEQ ID NO: 243 - protein, 85 aa, synthetic construct
MHHHHHHGGSGMLIFVKMLTGKTIELEVEPSDTIENVKAKIQDKEGIPPDQQRLAFQGKSLEDGRTLSDYNILKDSKKHPLLRLR

SEQ ID NO: 244 - protein, 85 aa, synthetic construct
MHHHHHHGGSGMLIFVKTLTGKMITLEVEPSDTIENVKAKIQDHEGIPPDQQRLAFQGKSLEDGRTLSDYNILKDSKLMPLLRLR

SEQ ID NO: 245 - protein, 514 aa, synthetic construct
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEENLYFQGHMNSFVGLRVVAKWSSNGYFYSGKITRDVGAGKYKLLFDDGYECDVLGKDILLCDPIPLDTEVTALSEDEYFSAGVVKGHRKESGELYYSIEKEGQRKWYKRMAVILSLEQGNRLREQYGLG

SEQ ID NO: 246 - protein, 85 aa, synthetic construct
MHHHHHHGGSGHLIFVKTLTGKTITLEVEPSDTIENVKAKIQDKEGIPPDQQRLAFAGKSLEDGRTLSDYNILKDSKLHPLLRLR

SEQ ID NO: 247 - protein, 85 aa, synthetic construct; identical to SEQ ID NO: 235

SEQ ID NO: 248 - protein, 85 aa, unidentified
MHHHHHHGGSGMLIFVKMLTGKTITLEVEPSDTIENVKAKIQDKEGIPPDQQRLAFAGKSLEDGRTLSDYNILKDSKLHPLLRLR

SEQ ID NO: 249 - protein, 85 aa, synthetic construct
MHHHHHHGGSGMLIFVKTLTGKMITLEVEPSDTIENVKAKIQDKEGIPPDQQRLAFAGKSLEDGRTLSDYNILKDSKLHPLLRLR

SEQ ID NO: 250 - protein, 85 aa, synthetic construct; identical to SEQ ID NO: 236

SEQ ID NO: 251 - protein, 85 aa, synthetic construct
MHHHHHHGGSGMLIFVKTLTGKTITLMVEPSDTIENVKAKIQDKEGIPPDQQRLAFAGKSLEDGRTLSDYNILKDSKLHPLLRLR

SEQ ID NO: 252 - protein, 85 aa, synthetic construct
MHHHHHHGGSGMLIFVKTLTGKTITLEVMPSDTIENVKAKIQDKEGIPPDQQRLAFAGKSLEDGRTLSDYNILKDSKLHPLLRLR

SEQ ID NO: 253 - protein, 85 aa, synthetic construct
MHHHHHHGGSGMLIFVKTLTGKTITLEVEPSDTIEVVKAKIQDKEGIPPDQQRLAFAGKSLEDGRTLSDYNILKDSKLHPLLRLR

SEQ ID NO: 254 - protein, 85 aa, synthetic construct
MHHHHHHGGSGMLIFVKTLTGKTITLEVEPSDTIENIKAKIQDKEGIPPDQQRLAFAGKSLEDGRTLSDYNILKDSKLHPLLRLR

SEQ ID NO: 255 - protein, 85 aa, synthetic construct
MHHHHHHGGSGMLIFVKTLTGKTITLEVEPSDTIENVKAKIWDKEGIPPDQQRLAFAGKSLEDGRTLSDYNILKDSKLHPLLRLR

SEQ ID NO: 256 - protein, 85 aa, synthetic construct
MHHHHHHGGSGMLIFVKTLTGKTITLEVEPSDTIENVKAKICDKEGIPPDQQRLAFAGKSLEDGRTLSDYNILKDSKLHPLLRLR

SEQ ID NO: 257 - protein, 85 aa, synthetic construct
MHHHHHHGGSGMLIFVKTLTGKTITLEVEPSDTIENVKAKIFDKEGIPPDQQRLAFAGKSLEDGRTLSDYNILKDSKLHPLLRLR

SEQ ID NO: 258 - protein, 85 aa, synthetic construct
MHHHHHHGGSGMLIFVKTLTGKTITLEVEPSDTIENVKAKIQDSEGIPPDQQRLAFAGKSLEDGRTLSDYNILKDSKLHPLLRLR

SEQ ID NO: 259 - protein, 85 aa, synthetic construct
MHHHHHHGGSGMLIFVKTLTGKTITLEVEPSDTIENVKAKIQDHEGIPPDQQRLAFAGKSLEDGRTLSDYNILKDSKLHPLLRLR

SEQ ID NO: 260 - protein, 85 aa, synthetic construct; identical to SEQ ID NO: 237

SEQ ID NO: 261 - protein, 85 aa, synthetic construct
MHHHHHHGGSGMLIFVKTLTGKTITLEVEPSDTIENVKAKIQDKEGIPLDQQRLAFAGKSLEDGRTLSDYNILKDSKLHPLLRLR

SEQ ID NO: 262 - protein, 85 aa, synthetic construct
MHHHHHHGGSGMLIFVKTLTGKTITLEVEPSDTIENVKAKIQDKEGIPPDQQWLAFAGKSLEDGRTLSDYNILKDSKLHPLLRLR

SEQ ID NO: 263 - protein, 85 aa, synthetic construct
MHHHHHHGGSGMLIFVKTLTGKTITLEVEPSDTIENVKAKIQDKEGIPPDQQRLTFAGKSLEDGRTLSDYNILKDSKLHPLLRLR

SEQ ID NO: 264 - protein, 85 aa, synthetic construct; identical to SEQ ID NO: 238

SEQ ID NO: 265 - protein, 85 aa, synthetic construct
MHHHHHHGGSGMLIFVKTLTGKTITLEVEPSDTIENVKAKIQDKEGIPPDQQRLAFAGTSLEDGRTLSDYNILKDSKLHPLLRLR

SEQ ID NO: 266 - protein, 85 aa, synthetic construct
MHHHHHHGGSGMLIFVKTLTGKTITLEVEPSDTIENVKAKIQDKEGIPPDQQRLAFAGKSLEDGYTLSDYNILKDSKLHPLLRLR

SEQ ID NO: 267 - protein, 85 aa, synthetic construct
MHHHHHHGGSGMLIFVKTLTGKTITLEVEPSDTIENVKAKIQDKEGIPPDQQRLAFAGKSLEDGRTLGDYNILKDSKLHPLLRLR

SEQ ID NO: 268 - protein, 85 aa, synthetic construct; identical to SEQ ID NO: 239

SEQ ID NO: 269 - protein, 85 aa, synthetic construct; identical to SEQ ID NO: 240

SEQ ID NO: 270 - protein, 85 aa, synthetic construct
MHHHHHHGGSGMLIFVKMLTGKMIELEVEPSDTIENVKAKIQDHEGIPPDQQRLAFQGKSLEDGRTLSDYNILKDPKKMPLLRLR

SEQ ID NO: 271 - protein, 85 aa, synthetic construct
MHHHHHHGGSGMLIFVRTLTGKMIELEVEPSDTIENVKAKIQDHEGIPPDQQRLAFQGKSLEDGRTLSDYNILKDPKKMPLLRLR

SEQ ID NO: 272 - protein, 85 aa, synthetic construct
MHHHHHHGGSGMLIFVRMLTGKTIELEVEPSDTIENVKAKIQDHEGIPPDQQRLAFQGKSLEDGRTLSDYNILKDPKKMPLLRLR

SEQ ID NO: 273 - protein, 85 aa, synthetic construct
MHHHHHHGGSGMLIFVRMLTGKMITLEVEPSDTIENVKAKIQDHEGIPPDQQRLAFQGKSLEDGRTLSDYNILKDPKKMPLLRLR

SEQ ID NO: 274 - protein, 85 aa, synthetic construct
MHHHHHHGGSGMLIFVRMLTGKMIELEVEPSDTIENVKAKIQDKEGIPPDQQRLAFQGKSLEDGRTLSDYNILKDPKKMPLLRLR

SEQ ID NO: 275 - protein, 85 aa, synthetic construct
MHHHHHHGGSGMLIFVRMLTGKMIELEVEPSDTIENVKAKIQDHEGIPPDQQRLAFAGKSLEDGRTLSDYNILKDPKKMPLLRLR

SEQ ID NO: 276 - protein, 85 aa, synthetic construct
MHHHHHHGGSGMLIFVRMLTGKMIELEVEPSDTIENVKAKIQDHEGIPPDQQRLAFQGKSLEDGRTLSDYNILKDSKKMPLLRLR

SEQ ID NO: 277 - protein, 85 aa, synthetic construct
MHHHHHHGGSGMLIFVRMLTGKMIELEVEPSDTIENVKAKIQDHEGIPPDQQRLAFQGKSLEDGRTLSDYNILKDPKLMPLLRLR

SEQ ID NO: 278 - protein, 85 aa, synthetic construct
MHHHHHHGGSGMLIFVRMLTGKMIELEVEPSDTIENVKAKIQDHEGIPPDQQRLAFQGKSLEDGRTLSDYNILKDPKKHPLLRLR

SEQ ID NO: 279 - protein, 85 aa, synthetic construct
MHHHHHHGGSGHLIFVRMLTGKMIELEVEPSDTIENVKAKIQDHEGIPPDQQRLAFQGKSLEDGRTLSDYNILKDPKKMPLLRLR

SEQ ID NO: 280 - protein, 85 aa, synthetic construct
MHHHHHHGGSGYLIFVRMLTGKMIELEVEPSDTIENVKAKIQDHEGIPPDQQRLAFQGKSLEDGRTLSDYNILKDPKKMPLLRLR

SEQ ID NO: 281 - protein, 85 aa, synthetic construct
MHHHHHHGGSGMLIFVRMLTGKMIHLEVEPSDTIENVKAKIQDHEGIPPDQQRLAFQGKSLEDGRTLSDYNILKDPKKMPLLRLR

SEQ ID NO: 282 - protein, 85 aa, synthetic construct
MHHHHHHGGSGMLIFVRMLTGKMIDLEVEPSDTIENVKAKIQDHEGIPPDQQRLAFQGKSLEDGRTLSDYNILKDPKKMPLLRLR

SEQ ID NO: 283 - protein, 85 aa, synthetic construct
MHHHHHHGGSGMLIFVRMLTGKMIELMVEPSDTIENVKAKIQDHEGIPPDQQRLAFQGKSLEDGRTLSDYNILKDPKKMPLLRLR

SEQ ID NO: 284 - protein, 85 aa, synthetic construct
MHHHHHHGGSGMLIFVRMLTGKMIELTVEPSDTIENVKAKIQDHEGIPPDQQRLAFQGKSLEDGRTLSDYNILKDPKKMPLLRLR

SEQ ID NO: 285 - protein, 85 aa, synthetic construct
MHHHHHHGGSGMLIFVRMLTGKMIELEVMPSDTIENVKAKIQDHEGIPPDQQRLAFQGKSLEDGRTLSDYNILKDPKKMPLLRLR

SEQ ID NO: 286 - protein, 85 aa, synthetic construct
MHHHHHHGGSGMLIFVRMLTGKMIELEVYPSDTIENVKAKIQDHEGIPPDQQRLAFQGKSLEDGRTLSDYNILKDPKKMPLLRLR

SEQ ID NO: 287 - protein, 85 aa, synthetic construct
MHHHHHHGGSGMLIFVRMLTGKMIELEVLPSDTIENVKAKIQDHEGIPPDQQRLAFQGKSLEDGRTLSDYNILKDPKKMPLLRLR

SEQ ID NO: 288 - protein, 85 aa, synthetic construct
MHHHHHHGGSGMLIFVRMLTGKMIELEVFPSDTIENVKAKIQDHEGIPPDQQRLAFQGKSLEDGRTLSDYNILKDPKKMPLLRLR

SEQ ID NO: 289 - protein, 85 aa, synthetic construct
MHHHHHHGGSGMLIFVRMLTGKMIELEVEPSDTIEVVKAKIQDHEGIPPDQQRLAFQGKSLEDGRTLSDYNILKDPKKMPLLRLR

SEQ ID NO: 290 - protein, 85 aa, synthetic construct
MHHHHHHGGSGMLIFVRMLTGKMIELEVEPSDTIEEVKAKIQDHEGIPPDQQRLAFQGKSLEDGRTLSDYNILKDPKKMPLLRLR

SEQ ID NO: 291 - protein, 85 aa, synthetic construct
MHHHHHHGGSGMLIFVRMLTGKMIELEVEPSDTIENIKAKIQDHEGIPPDQQRLAFQGKSLEDGRTLSDYNILKDPKKMPLLRLR

SEQ ID NO: 292 - protein, 85 aa, synthetic construct
MHHHHHHGGSGMLIFVRMLTGKMIELEVEPSDTIENVKAKICDHEGIPPDQQRLAFQGKSLEDGRTLSDYNILKDPKKMPLLRLR

SEQ ID NO: 293 - protein, 85 aa, synthetic construct
MHHHHHHGGSGMLIFVRMLTGKMIELEVEPSDTIENVKAKIWDHEGIPPDQQRLAFQGKSLEDGRTLSDYNILKDPKKMPLLRLR

SEQ ID NO: 294 - protein, 85 aa, synthetic construct
MHHHHHHGGSGMLIFVRMLTGKMIELEVEPSDTIENVKAKIFDHEGIPPDQQRLAFQGKSLEDGRTLSDYNILKDPKKMPLLRLR

SEQ ID NO: 295 - protein, 85 aa, synthetic construct
MHHHHHHGGSGMLIFVRMLTGKMIELEVEPSDTIENVKAKIQAHEGIPPDQQRLAFQGKSLEDGRTLSDYNILKDPKKMPLLRLR

SEQ ID NO: 296 - protein, 85 aa, synthetic construct
MHHHHHHGGSGMLIFVRMLTGKMIELEVEPSDTIENVKAKIQDSEGIPPDQQRLAFQGKSLEDGRTLSDYNILKDPKKMPLLRLR

SEQ ID NO: 297 - protein, 85 aa, synthetic construct
MHHHHHHGGSGMLIFVRMLTGKMIELEVEPSDTIENVKAKIQDQEGIPPDQQRLAFQGKSLEDGRTLSDYNILKDPKKMPLLRLR

SEQ ID NO: 298 - protein, 85 aa, synthetic construct
MHHHHHHGGSGMLIFVRMLTGKMIELEVEPSDTIENVKAKIQDAEGIPPDQQRLAFQGKSLEDGRTLSDYNILKDPKKMPLLRLR

SEQ ID NO: 299 - protein, 85 aa, synthetic construct
MHHHHHHGGSGMLIFVRMLTGKMIELEVEPSDTIENVKAKIQDHEGIPLDQQRLAFQGKSLEDGRTLSDYNILKDPKKMPLLRLR

SEQ ID NO: 300 - protein, 85 aa, synthetic construct
MHHHHHHGGSGMLIFVRMLTGKMIELEVEPSDTIENVKAKIQDHEGIPCDQQRLAFQGKSLEDGRTLSDYNILKDPKKMPLLRLR

SEQ ID NO: 301 - protein, 85 aa, synthetic construct
MHHHHHHGGSGMLIFVRMLTGKMIELEVEPSDTIENVKAKIQDHEGIPPDEQRLAFQGKSLEDGRTLSDYNILKDPKKMPLLRLR

SEQ ID NO: 302 - protein, 85 aa, synthetic construct
MHHHHHHGGSGMLIFVRMLTGKMIELEVEPSDTIENVKAKIQDHEGIPPDQQHLAFQGKSLEDGRTLSDYNILKDPKKMPLLRLR

SEQ ID NO: 303 - protein, 85 aa, synthetic construct
MHHHHHHGGSGMLIFVRMLTGKMIELEVEPSDTIENVKAKIQDHEGIPPDQQFLAFQGKSLEDGRTLSDYNILKDPKKMPLLRLR

SEQ ID NO: 304 - protein, 85 aa, synthetic construct
MHHHHHHGGSGMLIFVRMLTGKMIELEVEPSDTIENVKAKIQDHEGIPPDQQRLTFQGKSLEDGRTLSDYNILKDPKKMPLLRLR

SEQ ID NO: 305 - protein, 85 aa, synthetic construct
MHHHHHHGGSGMLIFVRMLTGKMIELEVEPSDTIENVKAKIQDHEGIPPDQQRLAFQGTSLEDGRTLSDYNILKDPKKMPLLRLR

SEQ ID NO: 306 - protein, 85 aa, synthetic construct
MHHHHHHGGSGMLIFVRMLTGKMIELEVEPSDTIENVKAKIQDHEGIPPDQQRLAFQGKLLEDGRTLSDYNILKDPKKMPLLRLR

SEQ ID NO: 307 - protein, 85 aa, synthetic construct
MHHHHHHGGSGMLIFVRMLTGKMIELEVEPSDTIENVKAKIQDHEGIPPDQQRLAFQGKMLEDGRTLSDYNILKDPKKMPLLRLR

SEQ ID NO: 308 - protein, 85 aa, synthetic construct
MHHHHHHGGSGMLIFVRMLTGKMIELEVEPSDTIENVKAKIQDHEGIPPDQQRLAFQGKSLDDGRTLSDYNILKDPKKMPLLRLR

SEQ ID NO: 309 - protein, 85 aa, synthetic construct
MHHHHHHGGSGMLIFVRMLTGKMIELEVEPSDTIENVKAKIQDHEGIPPDQQRLAFQGKSLEDGYTLSDYNILKDPKKMPLLRLR

SEQ ID NO: 310 - protein, 85 aa, synthetic construct
MHHHHHHGGSGMLIFVRMLTGKMIELEVEPSDTIENVKAKIQDHEGIPPDQQRLAFQGKSLEDGRTLGDYNILKDPKKMPLLRLR

SEQ ID NO: 311 - protein, 85 aa, synthetic construct
MHHHHHHGGSGMLIFVRMLTGKMIELEVEPSDTIENVKAKIQDHEGIPPDQQRLAFQGKSLEDGRTLSDYNLLKDPKKMPLLRLR

SEQ ID NO: 312 - protein, 85 aa, synthetic construct
MHHHHHHGGSGMLIFVRMLTGKMIELEVEPSDTIENVKAKIQDHEGIPPDQQRLAFQGKSLEDGRTLSDYNILIDPKKMPLLRLR

SEQ ID NO: 313 - protein, 85 aa, synthetic construct
MHHHHHHGGSGMLIFVRMLTGKMIELEVEPSDTIENVKAKIQDHEGIPPDQQRLAFQGKSLEDGRTLSDYNILKDHKKMPLLRLR

SEQ ID NO: 314 - protein, 85 aa, synthetic construct
MHHHHHHGGSGMLIFVRMLTGKMIELEVEPSDTIENVKAKIQDHEGIPPDQQRLAFQGKSLEDGRTLSDYNILKDPKKMPLLRMR

SEQ ID NO: 315 - protein, 85 aa, synthetic construct
MHHHHHHGGSGMLIFVRMLTGKMIELEVEPSDTIENVKAKIQDHEGIPPDQQRLAFQGKSLEDGRTLSDYNILKDPKKMPLLRLQ

SEQ ID NO: 316 - protein, 85 aa, synthetic construct
MHHHHHHGGSGMLIFVKMLTGKMIELEVEPSDTIENVKAKIQDHEGIPPDQQRLAFQGKSLEDGRTLSDYNILKDPKKMLVLRLR

SEQ ID NO: 317 - protein, 85 aa, synthetic construct
MHHHHHHGGSGMLIFVRMLTGKMIELEVYPSDTIENVKAKIQDHEGIPLDQQRLAFQGKLLEDGRTLGDYNILKDPKKMPLLRLR

SEQ ID NO: 318 - protein, 85 aa, synthetic construct
MHHHHHHGGSGHLIFVRMLTGKMIELEVEPSDTIENVKAKIQDHEGIPPDQQRLAFQGKSLEDGRTLSDYNILKDPKKMPLLRLQ

SEQ ID NO: 319 - protein, 85 aa, synthetic construct
MHHHHHHGGSGYLIFVRMLTGKMIELEVEPSDTIENIKAKIQDHEGIPPDQQRLAFQGKSLEDGRTLSDYNILKDPKKMPLLRMR

SEQ ID NO: 320 - protein, 85 aa, synthetic construct
MHHHHHHGGSGMLIFVRMLTGKMIELEVEPSDTIEVVKAKIQDHEGIPPDEQRLAFQGKSLDDGRTLSDYNILKDPKKMPLLRLR

SEQ ID NO: 321 - protein, 85 aa, synthetic construct
MHHHHHHGGSGMLIFVRMLTGKMIELEVEPSDTIENVKAKIQDHEGIPPDQQRLAFQGKSLEDGRTLSDYNLLIDPKKMPLLRLR

SEQ ID NO: 322 - protein, 85 aa, synthetic construct
MHHHHHHGGSGMLIFVRMLTGKMIELEVMPSDTIENVKAKIQDHEGIPPDQQRLAFQGTSLDDGRTLGDYNILKDPKKMPLLRLR

SEQ ID NO: 323 - protein, 85 aa, synthetic construct
MHHHHHHGGSGMLIFVRMLTGKMIELMVEPSDTIEVVKAKIQDHEGIPPDEQRLAFQGKLLEDGRTLSDYNILKDPKKMPLLRLR

SEQ ID NO: 324 - protein, 85 aa, synthetic construct
MHHHHHHGGSGMLIFVRMLTGKMIELEVEPSDTIENIKAKIQDHEGIPPDQYRLAFQGKLLEDGRTLGDYNILKDPKKMPLLRLR

SEQ ID NO: 325 - protein, 85 aa, synthetic construct
MHHHHHHGGSGMLIFVRMLTGKMIELEVEPSDTIENVKAKICDHEGIPPDQQHLAFQGKSLEDGRTLGDYNILKDPKKMPLLRLR

SEQ ID NO: 326 - protein, 85 aa, synthetic construct
MHHHHHHGGSGMLIFVRMLTGKMIELEVMPSDTIEVVKAKIFDHEGIPPDQQHLAFQGTSLEDGYTLGDYNILKDPKKMPLLRLR

SEQ ID NO: 327 - protein, 85 aa, synthetic construct
MHHHHHHGGSGMLIFVRMLTGKMIELEVMPSDTIEVVKAKIFDHEGIPPDQQHLTFQGTLLEDGYTLGDYNILKDPKKMPLLRLR

SEQ ID NO: 328 - protein, 85 aa, synthetic construct
MHHHHHHGGSGMLIFVRMLTGKMIELEVMPSDTIEVVKAKIFDHEGIPLDQQHLAFQGTSLEDGYTLGDYNILKDPKKMPLLRLR

SEQ ID NO: 329 - protein, 85 aa, synthetic construct
MHHHHHHGGSGMLIFVRMLTGKMIELEVMPSDTIEVVKAKIQDHEGIPPDQQHLAFQGTSLEDGYTLGDYNILKDPKKMPLLRLR

SEQ ID NO: 330 - protein, 85 aa, synthetic construct
MHHHHHHGGSGYLIFVRMLTGKMIELEVEPSDTIENIKAKIQDHEGIPPDQQRLAFQGKSLEDGRTLSDYNLLIDPKKMPLLRMR

SEQ ID NO: 331 - protein, 85 aa, synthetic construct
MHHHHHHGGSGYLIFVRMLTGKMIELEVEPSDTIEVIKAKIQDHEGIPPDEQRLAFQGKSLDDGRTLSDYNLLIDPKKMPLLRMR

SEQ ID NO: 332 - protein, 85 aa, synthetic construct
MHHHHHHGGSGMLIFVRMLTGKMIELEVEPSDTIEVVKAKIQDHEGIPPDEQRLAFQGKSLDDGRTLSDYNLLIDPKKMPLLRLR

SEQ ID NO: 333 - protein, 85 aa, synthetic construct
MHHHHHHGGSGMLIFVRMLTGKMIELEVMPSDTIENVKAKIQDHEGIPPDQQRLAFQGTSLDDGRTLGDYNLLIDPKKMPLLRLR

SEQ ID NO: 334 - protein, 85 aa, synthetic construct
MHHHHHHGGSGMLIFVRMLTGKMIELMVEPSDTIEVVKAKIQDHEGIPPDEQRLAFQGKLLEDGRTLSDYNLLIDPKKMPLLRLR

SEQ ID NO: 335 - protein, 85 aa, synthetic construct
MHHHHHHGGSGYLIFVRMLTGKMIELEVMPSDTIENIKAKIQDHEGIPPDQQRLAFQGTSLDDGRTLGDYNLLIDPKKMPLLRMR

SEQ ID NO: 336 - protein, 85 aa, synthetic construct
MHHHHHHGGSGYLIFVRMLTGKMIELEVMPSDTIENIKAKIQDHEGIPPDQQRLAFQGTSLDDGRTLGDYNLLIDPKKMPLLRMQ

SEQ ID NO: 337 - protein, 85 aa, synthetic construct
MHHHHHHGGSGYLIFVRMLTGKMIELEVMPSDTIEVIKAKIQDHEGIPPDEQRLAFQGTSLDDGRTLGDYNLLIDPKKMPLLRMQ

SEQ ID NO: 338 - protein, 85 aa, synthetic construct
MHHHHHHGGSGMLIFVRMLTGKMIELEVEPSDTIENVKAKIQDHEGIPPDQQRLTFQGKLLEDGRTLSDYNLLIDPKKMPLLRLR

SEQ ID NO: 339 - protein, 85 aa, synthetic construct
MHHHHHHGGSGMLIFVRMLTGKMIELEVEPSDTIENVKAKIQDHEGIPLDQQRLTFQGKLLEDGRTLSDYNLLIDPKKMPLLRLR

SEQ ID NO: 340 - protein, 85 aa, synthetic construct
MHHHHHHGGSGMLIFVRMLTGKMIELEVEPSDTIENVKAKIQDHEGIPLDQQRLTFQGKLLEDGRTLSDYNLLIDPKKMPLLRLQ

SEQ ID NO: 341 - protein, 85 aa, synthetic construct
MHHHHHHGGSGYLIFVRMLTGKMIELEVEPSDTIENIKAKIQDHEGIPLDQQRLTFQGKLLEDGRTLSDYNLLIDPKKMPLLRMQ

SEQ ID NO: 342 - protein, 85 aa, synthetic construct
MHHHHHHGGSGMLIFVRMLTGKMIELEVEPSDTIEVVKAKIQDHEGIPLDEQRLTFQGKLLDDGRTLSDYNLLIDPKKMPLLRLQ

SEQ ID NO: 343 - protein, 85 aa, synthetic construct
MHHHHHHGGSGYLIFVRMLTGKMIELEVEPSDTIEVIKAKIQDHEGIPLDEQRLTFQGKLLDDGRTLSDYNLLIDPKKMPLLRMQ

SEQ ID NO: 344 - protein, 85 aa, synthetic construct
MHHHHHHGGSGMLIFVRMLTGKMIELEVMPSDTIEVVKAKIQDHEGIPPDQQHLTFQGTLLEDGYTLGDYNLLIDPKKMPLLRLR

SEQ ID NO: 345 - protein, 85 aa, synthetic construct
MHHHHHHGGSGMLIFVRMLTGKMIELEVMPSDTIEVVKAKIQDHEGIPLDQQHLTFQGTLLEDGYTLGDYNLLIDPKKMPLLRLR

SEQ ID NO: 346 - protein, 85 aa, synthetic construct
MHHHHHHGGSGMLIFVRMLTGKMIELEVMPSDTIEVVKAKIQDHEGIPLDQQHLTFQGTLLEDGYTLGDYNLLIDPKKMPLLRLQ

SEQ ID NO: 347 - protein, 85 aa, synthetic construct
MHHHHHHGGSGMLIFVRMLTGKMIELEVMPSDTIEVVKAKIQDHEGIPLDEQHLTFQGTLLDDGYTLGDYNLLIDPKKMPLLRLQ

SEQ ID NO: 348 - protein, 85 aa, synthetic construct
MHHHHHHGGSGYLIFVRMLTGKMIELEVMPSDTIEVIKAKIQDHEGIPLDEQHLTFQGTLLDDGYTLGDYNLLIDPKKMPLLRMQ

SEQ ID NO: 349 - protein, 85 aa, synthetic construct
MHHHHHHGGSGYLIFVRMLTGKMIELEVMPSDTIEVIKAKIQDHEGIPPDEQRLAFQGTSLDDGRTLGDYNLLIDPKKMLVLRMQ

SEQ ID NO: 350 - protein, 85 aa, synthetic construct
MHHHHHHGGSGYLIFVRMLTGKMIELEVEPSDTIENIKAKIQDHEGIPLDQQRLTFQGKLLEDGRTLSDYNLLIDPKKMLVLRMQ

SEQ ID NO: 351 - protein, 85 aa, synthetic construct
MHHHHHHGGSGMLIFVRMLTGKMIELEVEPSDTIEVVKAKIQDHEGIPLDEQRLTFQGKLLDDGRTLSDYNLLIDPKKMLVLRLQ

SEQ ID NO: 352 - protein, 85 aa, synthetic construct
MHHHHHHGGSGMLIFVRMLTGKMIELEVMPSDTIEVVKAKIQDHEGIPLDQQHLTFQGTLLEDGYTLGDYNLLIDPKKMLVLRLQ

SEQ ID NO: 353 - protein, 85 aa, synthetic construct
MHHHHHHGGSGYLIFVRMLTGKMIELEVMPSDTIEVIKAKIQDHEGIPLDEQHLTFQGTLLDDGYTLGDYNLLIDPKKMLVLRMQ

SEQ ID NO: 354 - protein, 85 aa, synthetic construct
MHHHHHHGGSGMLIFVRMLTGKMIELEVMPSDTIEVVKAKIQDHEGIPPDQQHLAFQGTSLEDGYTLGDYNLLIDPKKMLVLRLR

SEQ ID NO: 355 - protein, 85 aa, synthetic construct
MHHHHHHGGSGYLIFVRMLTGKMIELEVEPSDTIENIKAKIQDHEGIPLDQQRLTFQGKLLEDGRTLSDYNLLIDPKKMAVLRMQ

SEQ ID NO: 356 - protein, 85 aa, synthetic construct
MHHHHHHGGSGYLIFVRMLTGKMIELEVEPSDTIENIKAKIQDHEGIPLDQQRLTFQGKLLEDGRTLSDYNLLIDPKKMRVLRMQ

SEQ ID NO: 357 - protein, 85 aa, synthetic construct
MHHHHHHGGSGYLIFVRMLTGKMIELEVEPSDTIENIKAKIQDHEGIPLDQQRLTFQGKLLEDGRTLSDYNLLIDPKKMNVLRMQ

SEQ ID NO: 358 - protein, 85 aa, synthetic construct
MHHHHHHGGSGYLIFVRMLTGKMIELEVEPSDTIENIKAKIQDHEGIPLDQQRLTFQGKLLEDGRTLSDYNLLIDPKKMDVLRMQ

SEQ ID NO: 359 - protein, 85 aa, synthetic construct
MHHHHHHGGSGYLIFVRMLTGKMIELEVEPSDTIENIKAKIQDHEGIPLDQQRLTFQGKLLEDGRTLSDYNLLIDPKKMCVLRMQ

SEQ ID NO: 360 - protein, 85 aa, synthetic construct
MHHHHHHGGSGYLIFVRMLTGKMIELEVEPSDTIENIKAKIQDHEGIPLDQQRLTFQGKLLEDGRTLSDYNLLIDPKKMEVLRMQ

SEQ ID NO: 361 - protein, 85 aa, synthetic construct
MHHHHHHGGSGYLIFVRMLTGKMIELEVEPSDTIENIKAKIQDHEGIPLDQQRLTFQGKLLEDGRTLSDYNLLIDPKKMQVLRMQ

SEQ ID NO: 362 - protein, 85 aa, synthetic construct
MHHHHHHGGSGYLIFVRMLTGKMIELEVEPSDTIENIKAKIQDHEGIPLDQQRLTFQGKLLEDGRTLSDYNLLIDPKKMGVLRMQ

SEQ ID NO: 363 - protein, 85 aa, synthetic construct
MHHHHHHGGSGYLIFVRMLTGKMIELEVEPSDTIENIKAKIQDHEGIPLDQQRLTFQGKLLEDGRTLSDYNLLIDPKKMHVLRMQ

SEQ ID NO: 364 - protein, 85 aa, synthetic construct
MHHHHHHGGSGYLIFVRMLTGKMIELEVEPSDTIENIKAKIQDHEGIPLDQQRLTFQGKLLEDGRTLSDYNLLIDPKKMIVLRMQ

SEQ ID NO: 365 - protein, 85 aa, synthetic construct
MHHHHHHGGSGYLIFVRMLTGKMIELEVEPSDTIENIKAKIQDHEGIPLDQQRLTFQGKLLEDGRTLSDYNLLIDPKKMKVLRMQ

SEQ ID NO: 366 - protein, 85 aa, synthetic construct
MHHHHHHGGSGYLIFVRMLTGKMIELEVEPSDTIENIKAKIQDHEGIPLDQQRLTFQGKLLEDGRTLSDYNLLIDPKKMMVLRMQ

SEQ ID NO: 367 - protein, 85 aa, synthetic construct
MHHHHHHGGSGYLIFVRMLTGKMIELEVEPSDTIENIKAKIQDHEGIPLDQQRLTFQGKLLEDGRTLSDYNLLIDPKKMFVLRMQ

SEQ ID NO: 368 - protein, 85 aa, synthetic construct
MHHHHHHGGSGYLIFVRMLTGKMIELEVEPSDTIENIKAKIQDHEGIPLDQQRLTFQGKLLEDGRTLSDYNLLIDPKKMSVLRMQ

SEQ ID NO: 369 - protein, 85 aa, synthetic construct
MHHHHHHGGSGYLIFVRMLTGKMIELEVEPSDTIENIKAKIQDHEGIPLDQQRLTFQGKLLEDGRTLSDYNLLIDPKKMTVLRMQ

SEQ ID NO: 370 - protein, 85 aa, synthetic construct
MHHHHHHGGSGYLIFVRMLTGKMIELEVEPSDTIENIKAKIQDHEGIPLDQQRLTFQGKLLEDGRTLSDYNLLIDPKKMWVLRMQ

SEQ ID NO: 371 - protein, 85 aa, synthetic construct
MHHHHHHGGSGYLIFVRMLTGKMIELEVEPSDTIENIKAKIQDHEGIPLDQQRLTFQGKLLEDGRTLSDYNLLIDPKKMYVLRMQ

SEQ ID NO: 372 - protein, 85 aa, synthetic construct
MHHHHHHGGSGYLIFVRMLTGKMIELEVEPSDTIENIKAKIQDHEGIPLDQQRLTFQGKLLEDGRTLSDYNLLIDPKKMVVLRMQ

SEQ ID NO: 373 - protein, 85 aa, synthetic construct
MHHHHHHGGSGYLIFVRMLTGKMIELEVEPSDTIENIKAKIQDHEGIPLDQQRLTFQGKLLEDGRTLSDYNLLIDPKKMLALRMQ

SEQ ID NO: 374 - protein, 85 aa, synthetic construct
MHHHHHHGGSGYLIFVRMLTGKMIELEVEPSDTIENIKAKIQDHEGIPLDQQRLTFQGKLLEDGRTLSDYNLLIDPKKMLRLRMQ

SEQ ID NO: 375 - protein, 85 aa, synthetic construct
MHHHHHHGGSGYLIFVRMLTGKMIELEVEPSDTIENIKAKIQDHEGIPLDQQRLTFQGKLLEDGRTLSDYNLLIDPKKMLNLRMQ

SEQ ID NO: 376 - protein, 85 aa, synthetic construct
MHHHHHHGGSGYLIFVRMLTGKMIELEVEPSDTIENIKAKIQDHEGIPLDQQRLTFQGKLLEDGRTLSDYNLLIDPKKMLDLRMQ

SEQ ID NO: 377 - protein, 85 aa, synthetic construct
MHHHHHHGGSGYLIFVRMLTGKMIELEVEPSDTIENIKAKIQDHEGIPLDQQRLTFQGKLLEDGRTLSDYNLLIDPKKMLCLRMQ

SEQ ID NO: 378 - protein, 85 aa, synthetic construct
MHHHHHHGGSGYLIFVRMLTGKMIELEVEPSDTIENIKAKIQDHEGIPLDQQRLTFQGKLLEDGRTLSDYNLLIDPKKMLELRMQ

SEQ ID NO: 379 - protein, 85 aa, synthetic construct
MHHHHHHGGSGYLIFVRMLTGKMIELEVEPSDTIENIKAKIQDHEGIPLDQQRLTFQGKLLEDGRTLSDYNLLIDPKKMLQLRMQ

SEQ ID NO: 380 - protein, 85 aa, synthetic construct
MHHHHHHGGSGYLIFVRMLTGKMIELEVEPSDTIENIKAKIQDHEGIPLDQQRLTFQGKLLEDGRTLSDYNLLIDPKKMLGLRMQ

SEQ ID NO: 381 - protein, 85 aa, synthetic construct
MHHHHHHGGSGYLIFVRMLTGKMIELEVEPSDTIENIKAKIQDHEGIPLDQQRLTFQGKLLEDGRTLSDYNLLIDPKKMLHLRMQ

SEQ ID NO: 382 - protein, 85 aa, synthetic construct
MHHHHHHGGSGYLIFVRMLTGKMIELEVEPSDTIENIKAKIQDHEGIPLDQQRLTFQGKLLEDGRTLSDYNLLIDPKKMLILRMQ

SEQ ID NO: 383 - protein, 85 aa, synthetic construct
MHHHHHHGGSGYLIFVRMLTGKMIELEVEPSDTIENIKAKIQDHEGIPLDQQRLTFQGKLLEDGRTLSDYNLLIDPKKMLKLRMQ

SEQ ID NO: 384 - protein, 85 aa, synthetic construct
MHHHHHHGGSGYLIFVRMLTGKMIELEVEPSDTIENIKAKIQDHEGIPLDQQRLTFQGKLLEDGRTLSDYNLLIDPKKMLMLRMQ

SEQ ID NO: 385 - protein, 85 aa, synthetic construct
MHHHHHHGGSGYLIFVRMLTGKMIELEVEPSDTIENIKAKIQDHEGIPLDQQRLTFQGKLLEDGRTLSDYNLLIDPKKMLFLRMQ

SEQ ID NO: 386 - protein, 85 aa, synthetic construct
MHHHHHHGGSGYLIFVRMLTGKMIELEVEPSDTIENIKAKIQDHEGIPLDQQRLTFQGKLLEDGRTLSDYNLLIDPKKMLPLRMQ

SEQ ID NO: 387 - protein, 85 aa, synthetic construct
MHHHHHHGGSGYLIFVRMLTGKMIELEVEPSDTIENIKAKIQDHEGIPLDQQRLTFQGKLLEDGRTLSDYNLLIDPKKMLSLRMQ

SEQ ID NO: 388 - protein, 85 aa, synthetic construct
MHHHHHHGGSGYLIFVRMLTGKMIELEVEPSDTIENIKAKIQDHEGIPLDQQRLTFQGKLLEDGRTLSDYNLLIDPKKMLTLRMQ

SEQ ID NO: 389 - protein, 85 aa, synthetic construct
MHHHHHHGGSGYLIFVRMLTGKMIELEVEPSDTIENIKAKIQDHEGIPLDQQRLTFQGKLLEDGRTLSDYNLLIDPKKMLWLRMQ

SEQ ID NO: 390 - protein, 85 aa, synthetic construct
MHHHHHHGGSGYLIFVRMLTGKMIELEVEPSDTIENIKAKIQDHEGIPLDQQRLTFQGKLLEDGRTLSDYNLLIDPKKMLYLRMQ

SEQ ID NO: 391 - protein, 85 aa, synthetic construct
MHHHHHHGGSGYMIFVRMLTGKMIELEVEPSDTIENIKAKIQDHEGIPLDQQRLTFQGKLLEDGRTLSDYNLPISPEKMAVLRMQ

SEQ ID NO: 392 - protein, 85 aa, synthetic construct
MHHHHHHGGSGYMIFVRMLTGKMIELEVEPSDTIENIKAKIQDHEGIPLDQQRLTFQGKLLEDGRTLSDYNLPISPEKMGVLRMQ

SEQ ID NO: 393 - protein, 85 aa, synthetic construct
MHHHHHHGGSGYMIFVRMLTGKMIELEVEPSDTIENIKAKIQDHEGIPLDQQRLTFQGKLLEDGRTLSDYNLPISPEKMLMLRMQ

SEQ ID NO: 394 - protein, 85 aa, synthetic construct
MHHHHHHGGSGYMIFVRMLTGKMIELEVEPSDTIENIKAKIQDHEGIPLDQQRLTFQGKLLEDGRTLSDYNLPISPEKMAMLRMQ

SEQ ID NO: 395 - protein, 85 aa, synthetic construct
MHHHHHHGGSGYMIFVRMLTGKMIELEVEPSDTIENIKAKIQDHEGIPLDQQRLTFQGKLLEDGRTLSDYNLPISPEKMAFLRMQ

SEQ ID NO: 396 - protein, 85 aa, synthetic construct
MHHHHHHGGSGYMIFVRMLTGKMIELEVEPSDTIENIKAKIQDHEGIPLDQQRLTFQGKLLEDGRTLSDYNLPISPEKMACLRMQ

SEQ ID NO: 397 - protein, 85 aa, synthetic construct
MHHHHHHGGSGYMIFVRMLTGKMIELEVEPSDTIENIKAKIQDHEGIPLDQQRLTFQGKLLEDGRTLSDYNLPISPEKMGMLRMQ

SEQ ID NO: 398 - protein, 85 aa, synthetic construct
MHHHHHHGGSGYMIFVRMLTGKMIELEVEPSDTIENIKAKIQDHEGIPLDQQRLTFQGKLLEDGRTLSDYNLPISPEKMGFLRMQ

SEQ ID NO: 399 - protein, 85 aa, synthetic construct
MHHHHHHGGSGYMIFVRMLTGKMIELEVEPSDTIENIKAKIQDHEGIPLDQQRLTFQGKLLEDGRTLSDYNLPISPEKMGCLRMQ

SEQ ID NO: 400 - protein, 85 aa, synthetic construct
MHHHHHHGGSGYMIFVRMLTGKMIELEVEPSDTIENIKAKIQDHEGIPLDQQRLTFQGKLLEDGRTLSDYNLPISPEKMCMLRMQ

SEQ ID NO: 401 - protein, 85 aa, synthetic construct
MHHHHHHGGSGYMIFVRMLTGKMIELEVEPSDTIENIKAKIQDHEGIPLDQQRLTFQGKLLEDGRTLSDYNLPISPEKMMMLRMQ

SEQ ID NO: 402 - protein, 85 aa, synthetic construct
MHHHHHHGGSGYMIFVRMLTGKMIELEVEPSDTIENIKAKIQDHEGIPLDQQRLTFQGKLLEDGRTLSDYNLPISPEKMFMLRMQ

SEQ ID NO: 403 - protein, 85 aa, synthetic construct
MHHHHHHGGSGYMIFVRMLTGKMIELEVEPSDTIENIKAKIQDHEGIPLDQQRLTFQGKLLEDGRTLSDYNLAISPEKMAVLRMQ

SEQ ID NO: 404 - protein, 85 aa, synthetic construct
MHHHHHHGGSGYMIFVRMLTGKMIELEVEPSDTIENIKAKIQDHEGIPLDQQRLTFQGKLLEDGRTLSDYNLRISPEKMAVLRMQ

SEQ ID NO: 405 - protein, 85 aa, synthetic construct
MHHHHHHGGSGYMIFVRMLTGKMIELEVEPSDTIENIKAKIQDHEGIPLDQQRLTFQGKLLEDGRTLSDYNLNISPEKMAVLRMQ

SEQ ID NO: 406 - protein, 85 aa, synthetic construct
MHHHHHHGGSGYMIFVRMLTGKMIELEVEPSDTIENIKAKIQDHEGIPLDQQRLTFQGKLLEDGRTLSDYNLDISPEKMAVLRMQ

SEQ ID NO: 407 - protein, 85 aa, synthetic construct
MHHHHHHGGSGYMIFVRMLTGKMIELEVEPSDTIENIKAKIQDHEGIPLDQQRLTFQGKLLEDGRTLSDYNLCISPEKMAVLRMQ

SEQ ID NO: 408 - protein, 85 aa, synthetic construct
MHHHHHHGGSGYMIFVRMLTGKMIELEVEPSDTIENIKAKIQDHEGIPLDQQRLTFQGKLLEDGRTLSDYNLEISPEKMAVLRMQ

SEQ ID NO: 409 - protein, 85 aa, synthetic construct
MHHHHHHGGSGYMIFVRMLTGKMIELEVEPSDTIENIKAKIQDHEGIPLDQQRLTFQGKLLEDGRTLSDYNLQISPEKMAVLRMQ

SEQ ID NO: 410 - protein, 85 aa, synthetic construct
MHHHHHHGGSGYMIFVRMLTGKMIELEVEPSDTIENIKAKIQDHEGIPLDQQRLTFQGKLLEDGRTLSDYNLGISPEKMAVLRMQ

SEQ ID NO: 411 - protein, 85 aa, synthetic construct
MHHHHHHGGSGYMIFVRMLTGKMIELEVEPSDTIENIKAKIQDHEGIPLDQQRLTFQGKLLEDGRTLSDYNLHISPEKMAVLRMQ

SEQ ID NO: 412 - protein, 85 aa, synthetic construct
MHHHHHHGGSGYMIFVRMLTGKMIELEVEPSDTIENIKAKIQDHEGIPLDQQRLTFQGKLLEDGRTLSDYNLIISPEKMAVLRMQ

SEQ ID NO: 413 - protein, 85 aa, synthetic construct
MHHHHHHGGSGYMIFVRMLTGKMIELEVEPSDTIENIKAKIQDHEGIPLDQQRLTFQGKLLEDGRTLSDYNLKISPEKMAVLRMQ

SEQ ID NO: 414 - protein, 85 aa, synthetic construct
MHHHHHHGGSGYMIFVRMLTGKMIELEVEPSDTIENIKAKIQDHEGIPLDQQRLTFQGKLLEDGRTLSDYNLMISPEKMAVLRMQ

SEQ ID NO: 415 - protein, 85 aa, synthetic construct
MHHHHHHGGSGYMIFVRMLTGKMIELEVEPSDTIENIKAKIQDHEGIPLDQQRLTFQGKLLEDGRTLSDYNLFISPEKMAVLRMQ

SEQ ID NO: 416 - protein, 85 aa, synthetic construct
MHHHHHHGGSGYMIFVRMLTGKMIELEVEPSDTIENIKAKIQDHEGIPLDQQRLTFQGKLLEDGRTLSDYNLSISPEKMAVLRMQ

SEQ ID NO: 417 - protein, 85 aa, synthetic construct
MHHHHHHGGSGYMIFVRMLTGKMIELEVEPSDTIENIKAKIQDHEGIPLDQQRLTFQGKLLEDGRTLSDYNLTISPEKMAVLRMQ

SEQ ID NO: 418 - protein, 85 aa, synthetic construct
MHHHHHHGGSGYMIFVRMLTGKMIELEVEPSDTIENIKAKIQDHEGIPLDQQRLTFQGKLLEDGRTLSDYNLWISPEKMAVLRMQ

SEQ ID NO: 419 - protein, 85 aa, synthetic construct
MHHHHHHGGSGYMIFVRMLTGKMIELEVEPSDTIENIKAKIQDHEGIPLDQQRLTFQGKLLEDGRTLSDYNLYISPEKMAVLRMQ

SEQ ID NO: 420 - protein, 85 aa, synthetic construct
MHHHHHHGGSGYMIFVRMLTGKMIELEVEPSDTIENIKAKIQDHEGIPLDQQRLTFQGKLLEDGRTLSDYNLVISPEKMAVLRMQ

SEQ ID NO: 421 - protein, 85 aa, synthetic construct
MHHHHHHGGSGYMIFVRMLTGKMIELEVEPSDTIENIKAKIQDHEGIPLDQQRLTFQGKLLEDGRTLSDYNLPISPEKMCFLRMQ

SEQ ID NO: 422 - protein, 85 aa, synthetic construct
MHHHHHHGGSGYMIFVRMLTGKMIELEVEPSDTIENIKAKIQDHEGIPLDQQRLTFQGKLLEDGRTLSDYNLPISPEKMMFLRMQ

SEQ ID NO: 423 - protein, 85 aa, synthetic construct
MHHHHHHGGSGYMIFVRMLTGKMIELEVEPSDTIENIKAKIQDHEGIPLDQQRLTFQGKLLEDGRTLSDYNLPISPEKMFFLRMQ

SEQ ID NO: 424 - protein, 85 aa, synthetic construct
MHHHHHHGGSGYMIFVRMLTGKMIELEVEPSDTIENIKAKIQDHEGIPLDQQRLTFQGKLLEDGRTLSDYNLPISPEKMCCLRMQ

SEQ ID NO: 425 - protein, 85 aa, synthetic construct
MHHHHHHGGSGYMIFVRMLTGKMIELEVEPSDTIENIKAKIQDHEGIPLDQQRLTFQGKLLEDGRTLSDYNLPISPEKMMCLRMQ

SEQ ID NO: 426 - protein, 85 aa, synthetic construct
MHHHHHHGGSGYMIFVRMLTGKMIELEVEPSDTIENIKAKIQDHEGIPLDQQRLTFQGKLLEDGRTLSDYNLPISPEKMFCLRMQ

SEQ ID NO: 427 - protein, 85 aa, synthetic construct
MHHHHHHGGSGYMIFVRMLTGKMIELEVEPSDTIENIKAKIQDHEGIPLDQQRLTFQGKLLEDGRTLSDYNLAISPEKMAMLRMQ

SEQ ID NO: 428 - protein, 85 aa, synthetic construct
MHHHHHHGGSGYMIFVRMLTGKMIELEVEPSDTIENIKAKIQDHEGIPLDQQRLTFQGKLLEDGRTLSDYNLCISPEKMAMLRMQ

SEQ ID NO: 429 - protein, 85 aa, synthetic construct
MHHHHHHGGSGYMIFVRMLTGKMIELEVEPSDTIENIKAKIQDHEGIPLDQQRLTFQGKLLEDGRTLSDYNLTISPEKMAMLRMQ

SEQ ID NO: 430 - protein, 85 aa, synthetic construct
MHHHHHHGGSGYMIFVRMLTGKMIELEVEPSDTIENIKAKIQDHEGIPLDQQRLTFQGKLLEDGRTLSDYNLVISPEKMAMLRMQ

SEQ ID NO: 431 - protein, 85 aa, synthetic construct
MHHHHHHGGSGYAIFVRMLTGKMIELEVEPSDTIENIKAKIQDHEGIPLDQQRLTFQGKLLEDGRTLSDYNLTISPEKMAVLRMQ

SEQ ID NO: 432 - protein, 85 aa, synthetic construct
MHHHHHHGGSGYRIFVRMLTGKMIELEVEPSDTIENIKAKIQDHEGIPLDQQRLTFQGKLLEDGRTLSDYNLTISPEKMAVLRMQ

SEQ ID NO: 433 - protein, 85 aa, synthetic construct
MHHHHHHGGSGYNIFVRMLTGKMIELEVEPSDTIENIKAKIQDHEGIPLDQQRLTFQGKLLEDGRTLSDYNLTISPEKMAVLRMQ

SEQ ID NO: 434 - protein, 85 aa, synthetic construct
MHHHHHHGGSGYDIFVRMLTGKMIELEVEPSDTIENIKAKIQDHEGIPLDQQRLTFQGKLLEDGRTLSDYNLTISPEKMAVLRMQ

SEQ ID NO: 435 - protein, 85 aa, synthetic construct
MHHHHHHGGSGYCIFVRMLTGKMIELEVEPSDTIENIKAKIQDHEGIPLDQQRLTFQGKLLEDGRTLSDYNLTISPEKMAVLRMQ

SEQ ID NO: 436 - protein, 85 aa, synthetic construct
MHHHHHHGGSGYEIFVRMLTGKMIELEVEPSDTIENIKAKIQDHEGIPLDQQRLTFQGKLLEDGRTLSDYNLTISPEKMAVLRMQ

SEQ ID NO: 437 - protein, 85 aa, synthetic construct
MHHHHHHGGSGYQIFVRMLTGKMIELEVEPSDTIENIKAKIQDHEGIPLDQQRLTFQGKLLEDGRTLSDYNLTISPEKMAVLRMQ

SEQ ID NO: 438 - protein, 85 aa, synthetic construct
MHHHHHHGGSGYGIFVRMLTGKMIELEVEPSDTIENIKAKIQDHEGIPLDQQRLTFQGKLLEDGRTLSDYNLTISPEKMAVLRMQ

SEQ ID NO: 439 - protein, 85 aa, synthetic construct
MHHHHHHGGSGYHIFVRMLTGKMIELEVEPSDTIENIKAKIQDHEGIPLDQQRLTFQGKLLEDGRTLSDYNLTISPEKMAVLRMQ

SEQ ID NO: 440 - protein, 85 aa, synthetic construct
MHHHHHHGGSGYIIFVRMLTGKMIELEVEPSDTIENIKAKIQDHEGIPLDQQRLTFQGKLLEDGRTLSDYNLTISPEKMAVLRMQ

SEQ ID NO: 441 - protein, 85 aa, synthetic construct
MHHHHHHGGSGYKIFVRMLTGKMIELEVEPSDTIENIKAKIQDHEGIPLDQQRLTFQGKLLEDGRTLSDYNLTISPEKMAVLRMQ

SEQ ID NO: 442 - protein, 85 aa, synthetic construct
MHHHHHHGGSGYLIFVRMLTGKMIELEVEPSDTIENIKAKIQDHEGIPLDQQRLTFQGKLLEDGRTLSDYNLTISPEKMAVLRMQ

SEQ ID NO: 443 - protein, 85 aa, synthetic construct
MHHHHHHGGSGYFIFVRMLTGKMIELEVEPSDTIENIKAKIQDHEGIPLDQQRLTFQGKLLEDGRTLSDYNLTISPEKMAVLRMQ

SEQ ID NO: 444 - protein, 85 aa, synthetic construct
MHHHHHHGGSGYSIFVRMLTGKMIELEVEPSDTIENIKAKIQDHEGIPLDQQRLTFQGKLLEDGRTLSDYNLTISPEKMAVLRMQ

SEQ ID NO: 445 - protein, 85 aa, synthetic construct
MHHHHHHGGSGYTIFVRMLTGKMIELEVEPSDTIENIKAKIQDHEGIPLDQQRLTFQGKLLEDGRTLSDYNLTISPEKMAVLRMQ

SEQ ID NO: 446 - protein, 85 aa, synthetic construct
MHHHHHHGGSGYWIFVRMLTGKMIELEVEPSDTIENIKAKIQDHEGIPLDQQRLTFQGKLLEDGRTLSDYNLTISPEKMAVLRMQ

SEQ ID NO: 447 - protein, 85 aa, synthetic construct
MHHHHHHGGSGYYIFVRMLTGKMIELEVEPSDTIENIKAKIQDHEGIPLDQQRLTFQGKLLEDGRTLSDYNLTISPEKMAVLRMQ

SEQ ID NO: 448 - protein, 85 aa, synthetic construct
MHHHHHHGGSGYVIFVRMLTGKMIELEVEPSDTIENIKAKIQDHEGIPLDQQRLTFQGKLLEDGRTLSDYNLTISPEKMAVLRMQ

SEQ ID NO: 449 - protein, 85 aa, synthetic construct
MHHHHHHGGSGYPIFVRMLTGKMIELEVEPSDTIENIKAKIQDHEGIPLDQQRLTFQGKLLEDGRTLSDYNLTISPEKMAVLRMQ

SEQ ID NO: 450 - protein, 74 aa, synthetic construct
  VAR_SEQ 1: note = X is selected from M, H, Y, W, Q, T, F, S, R, I, and N
  VAR_SEQ 2: note = X is selected from Q, L, I, and M
  VAR_SEQ 6: note = X is selected from K and R
  VAR_SEQ 7: note = X is selected from T, M, I, C, L, and V
  VAR_SEQ 9: note = X is selected from T, I, S, E and V
  VAR_SEQ 12: note = X is selected from T, M, and Y
  VAR_SEQ 13: note = X is selected from I, F, H and P
  VAR_SEQ 14: note = X is selected from T, E, D, H, and N
  VAR_SEQ 16: note = X is selected from E, M, T, N, Y, D, and H
  VAR_SEQ 17: note = X is selected from V and C
  VAR_SEQ 18: note = X is selected from E, M, Y, L, H, F, W, S, Q, T, C, N, R, and D
  VAR_SEQ 19: note = X is selected from P and K
  VAR_SEQ 20: note = X is selected from S, D, N, C, A, and W
  VAR_SEQ 21: note = X is selected from D and E
  VAR_SEQ 25: note = X is selected from N, V, I, E, G, M, Q, D, A, L, R, S, K, T, C, and F
  VAR_SEQ 26: note = X is selected from I, V, and L
  VAR_SEQ 28: note = X is selected from A, E, Q, W, I, M, and D
  VAR_SEQ 29: note = X is selected from K, M, L, R, Q, and H
  VAR_SEQ 31: note = X is selected from Q, C, F, W, H, Y, L, R, and M
  VAR_SEQ 32: note = X is selected from D, A, E, and R
  VAR_SEQ 33: note = X is selected from K, H, A, Q, S, V, L, E, M, T, I, F, C, Y, R, N, and W
  VAR_SEQ 34: note = X is selected from E and T
  VAR_SEQ 38: note = X is selected from P, L, C, F, I, V, Y, T, M, H, S, Q, A, W, N, and K
  VAR_SEQ 39: note = X is selected from D, W, E, G, S, L, and Q
  VAR_SEQ 40: note = X is selected from Q, E, and D
  VAR_SEQ 41: note = X is selected from Q, Y, I, C, and V
  VAR_SEQ 42: note = X is selected from R, W, F, H, Y, N, C, and S
  VAR_SEQ 44: note = X is selected from I, A and T
  VAR_SEQ 46: note = X is selected from A, Q, and G
  VAR_SEQ 48: note = X is selected from K, T, M, I, Q, V, R, L, and N
  VAR_SEQ 49: note = X is selected from Q, S, L, M, P, E V, A, D, I, C, G, and N
  VAR_SEQ 51: note = X is selected from E and D
  VAR_SEQ 52: note = X is selected from D and E
  VAR_SEQ 54: note = X is selected from R, Y, M, T, H, F, N, Q, K, and C
  VAR_SEQ 55: note = X is selected from T and R
  VAR_SEQ 57: note = X is selected from S, G, D, N, H, E, A, Q, M, R, and K
  VAR_SEQ 58: note = X is selected from D and S
  VAR_SEQ 60: note = X is selected from N, E, and Q
  VAR_SEQ 61: note = X is selected from I and L
  VAR_SEQ 62: note = X is selected from Q, L, T, V, C, A, M, I and S
  VAR_SEQ 63: note = X is selected from K, I, M, F, and V
  VAR_SEQ 64: note = X is selected from E, D, and S
  VAR_SEQ 65: note = X is selected from S, P, E, K, H, R, A, D, N, and Q
  VAR_SEQ 66: note = X is selected from T, K, R, and E
  VAR_SEQ 67: note = X is selected from L, H, K, R, S, M, C, Y, and T
  VAR_SEQ 68: note = X is selected from H, M, Q, and E
  VAR_SEQ 69: note = X is selected from L, P, R, A, G, C, F, M, and S
  VAR_SEQ 70: note = X is selected from V, L, M, F, and C
  VAR_SEQ 73: note = X is selected from L and M
  VAR_SEQ 74: note = X is selected from R, Q, V, L, M, C, I, T, E, and K
XXIFVXXLXGKXXXLXXXXXXTIEXXKXXIXXXXGIPXXXXXLXFXGXXLXXGXXLXXYXXXXXXXXXXXLRXX

SEQ ID NO: 451 - protein, 74 aa, synthetic construct
MLIFVKTLTGKTITLEVEPSDTIENVKAKIQDKEGIPPDQQRLAFAGKSLEDGRTLSDYNILKDSKLHLVLRLR

SEQ ID NO: 452 - protein, 74 aa, synthetic construct; identical to SEQ ID NO: 14

SEQ ID NO: 453 - protein, 74 aa, synthetic construct; identical to SEQ ID NO: 30

SEQ ID NO: 454 - protein, 74 aa, synthetic construct; identical to SEQ ID NO: 100

SEQ ID NO: 455 - protein, 74 aa, synthetic construct; identical to SEQ ID NO: 151

SEQ ID NO: 456 - protein, 74 aa, synthetic construct; identical to SEQ ID NO: 201

SEQ ID NO: 457 - protein, 74 aa, synthetic construct; identical to SEQ ID NO: 202

SEQ ID NO: 458 - protein, 74 aa, synthetic construct; identical to SEQ ID NO: 4

SEQ ID NO: 459 - protein, 74 aa, synthetic construct; identical to SEQ ID NO: 14

SEQ ID NO: 460 - protein, 74 aa, synthetic construct; identical to SEQ ID NO: 15

SEQ ID NO: 461 - protein, 74 aa, synthetic construct; identical to SEQ ID NO: 24

SEQ ID NO: 462 - protein, 74 aa, synthetic construct; identical to SEQ ID NO: 30

SEQ ID NO: 463 - protein, 74 aa, synthetic construct; identical to SEQ ID NO: 34

SEQ ID NO: 464 - protein, 74 aa, synthetic construct; identical to SEQ ID NO: 41

SEQ ID NO: 465 - protein, 74 aa, synthetic construct; identical to SEQ ID NO: 64

SEQ ID NO: 466 - protein, 74 aa, synthetic construct; identical to SEQ ID NO: 75

SEQ ID NO: 467 - protein, 74 aa, synthetic construct; identical to SEQ ID NO: 89

SEQ ID NO: 468 - protein, 74 aa, synthetic construct; identical to SEQ ID NO: 88

SEQ ID NO: 469 - protein, 74 aa, synthetic construct; identical to SEQ ID NO: 93

SEQ ID NO: 470 - protein, 74 aa, synthetic construct; identical to SEQ ID NO: 105

SEQ ID NO: 471 - protein, 74 aa, synthetic construct; identical to SEQ ID NO: 99

SEQ ID NO: 472 - protein, 74 aa, synthetic construct; identical to SEQ ID NO: 100

SEQ ID NO: 473 - protein, 74 aa, synthetic construct; identical to SEQ ID NO: 116

SEQ ID NO: 474 - protein, 74 aa, synthetic construct; identical to SEQ ID NO: 146

SEQ ID NO: 475 - protein, 74 aa, synthetic construct; identical to SEQ ID NO: 150

SEQ ID NO: 476 - protein, 74 aa, synthetic construct; identical to SEQ ID NO: 151

SEQ ID NO: 477 - protein, 74 aa, synthetic construct; identical to SEQ ID NO: 154

SEQ ID NO: 478 - protein, 74 aa, synthetic construct; identical to SEQ ID NO: 178

SEQ ID NO: 479 - protein, 74 aa, synthetic construct; identical to SEQ ID NO: 185

SEQ ID NO: 480 - protein, 74 aa, synthetic construct; identical to SEQ ID NO: 201

SEQ ID NO: 481 - protein, 74 aa, synthetic construct; identical to SEQ ID NO: 202

SEQ ID NO: 482 - protein, 74 aa, synthetic construct
MLIFVRMLTGKMIELEVEPSDTIENVKAKIQDHEGIPPDQQRLAFQGKSLEDGRTLSDYNILKDPKKMPLLRLR

SEQ ID NO: 483 - protein, 74 aa, synthetic construct
MLIFVRTLTGKTITLEVEPSDTIENVKAKIQDHEGIPPDQQRLAFQGKSLEDGRTLSDYNILKDPKLHPLLRLR

SEQ ID NO: 484 - protein, 74 aa, synthetic construct
MLIFVKMLTGKTIELEVEPSDTIENVKAKIQDKEGIPPDQQRLAFQGKSLEDGRTLSDYNILKDSKKHPLLRLR

SEQ ID NO: 485 - protein, 74 aa, synthetic construct
MLIFVKTLTGKMITLEVEPSDTIENVKAKIQDHEGIPPDQQRLAFQGKSLEDGRTLSDYNILKDSKLMPLLRLR

SEQ ID NO: 486 - protein, 74 aa, synthetic construct
MLIFVKMLTGKMIELEVEPSDTIENVKAKIQDHEGIPPDQQRLAFQGKSLEDGRTLSDYNILKDPKKMPLLRLR

SEQ ID NO: 487 - protein, 74 aa, synthetic construct
MLIFVRTLTGKMIELEVEPSDTIENVKAKIQDHEGIPPDQQRLAFQGKSLEDGRTLSDYNILKDPKKMPLLRLR

SEQ ID NO: 488 - protein, 74 aa, synthetic construct
MLIFVRMLTGKTIELEVEPSDTIENVKAKIQDHEGIPPDQQRLAFQGKSLEDGRTLSDYNILKDPKKMPLLRLR

SEQ ID NO: 489 - protein, 74 aa, synthetic construct
MLIFVRMLTGKMITLEVEPSDTIENVKAKIQDHEGIPPDQQRLAFQGKSLEDGRTLSDYNILKDPKKMPLLRLR

SEQ ID NO: 490 - protein, 74 aa, synthetic construct
MLIFVRMLTGKMIELEVEPSDTIENVKAKIQDKEGIPPDQQRLAFQGKSLEDGRTLSDYNILKDPKKMPLLRLR

SEQ ID NO: 491 - protein, 74 aa, synthetic construct
MLIFVRMLTGKMIELEVEPSDTIENVKAKIQDHEGIPPDQQRLAFAGKSLEDGRTLSDYNILKDPKKMPLLRLR

SEQ ID NO: 492 - protein, 74 aa, synthetic construct
MLIFVRMLTGKMIELEVEPSDTIENVKAKIQDHEGIPPDQQRLAFQGKSLEDGRTLSDYNILKDSKKMPLLRLR

SEQ ID NO: 493 - protein, 74 aa, synthetic construct
MLIFVRMLTGKMIELEVEPSDTIENVKAKIQDHEGIPPDQQRLAFQGKSLEDGRTLSDYNILKDPKLMPLLRLR

SEQ ID NO: 494 - protein, 74 aa, synthetic construct
MLIFVRMLTGKMIELEVEPSDTIENVKAKIQDHEGIPPDQQRLAFQGKSLEDGRTLSDYNILKDPKKHPLLRLR

SEQ ID NO: 495 - protein, 74 aa, synthetic construct
HLIFVRMLTGKMIELEVEPSDTIENVKAKIQDHEGIPPDQQRLAFQGKSLEDGRTLSDYNILKDPKKMPLLRLR

SEQ ID NO: 496 - protein, 74 aa, synthetic construct
YLIFVRMLTGKMIELEVEPSDTIENVKAKIQDHEGIPPDQQRLAFQGKSLEDGRTLSDYNILKDPKKMPLLRLR

SEQ ID NO: 497 - protein, 74 aa, synthetic construct
MLIFVRMLTGKMIHLEVEPSDTIENVKAKIQDHEGIPPDQQRLAFQGKSLEDGRTLSDYNILKDPKKMPLLRLR

SEQ ID NO: 498 - protein, 74 aa, synthetic construct
MLIFVRMLTGKMIDLEVEPSDTIENVKAKIQDHEGIPPDQQRLAFQGKSLEDGRTLSDYNILKDPKKMPLLRLR

SEQ ID NO: 499 - protein, 74 aa, synthetic construct
MLIFVRMLTGKMIELMVEPSDTIENVKAKIQDHEGIPPDQQRLAFQGKSLEDGRTLSDYNILKDPKKMPLLRLR

SEQ ID NO: 500 - protein, 74 aa, synthetic construct
MLIFVRMLTGKMIELTVEPSDTIENVKAKIQDHEGIPPDQQRLAFQGKSLEDGRTLSDYNILKDPKKMPLLRLR

SEQ ID NO: 501 - protein, 74 aa, synthetic construct
MLIFVRMLTGKMIELEVMPSDTIENVKAKIQDHEGIPPDQQRLAFQGKSLEDGRTLSDYNILKDPKKMPLLRLR

SEQ ID NO: 502 - protein, 74 aa, synthetic construct
MLIFVRMLTGKMIELEVYPSDTIENVKAKIQDHEGIPPDQQRLAFQGKSLEDGRTLSDYNILKDPKKMPLLRLR

SEQ ID NO: 503 - protein, 74 aa, synthetic construct
MLIFVRMLTGKMIELEVLPSDTIENVKAKIQDHEGIPPDQQRLAFQGKSLEDGRTLSDYNILKDPKKMPLLRLR

SEQ ID NO: 504 - protein, 74 aa, synthetic construct
MLIFVRMLTGKMIELEVFPSDTIENVKAKIQDHEGIPPDQQRLAFQGKSLEDGRTLSDYNILKDPKKMPLLRLR

SEQ ID NO: 505 - protein, 74 aa, synthetic construct
MLIFVRMLTGKMIELEVEPSDTIEVVKAKIQDHEGIPPDQQRLAFQGKSLEDGRTLSDYNILKDPKKMPLLRLR

SEQ ID NO: 506 - protein, 74 aa, synthetic construct
MLIFVRMLTGKMIELEVEPSDTIEEVKAKIQDHEGIPPDQQRLAFQGKSLEDGRTLSDYNILKDPKKMPLLRLR

SEQ ID NO: 507 - protein, 74 aa, synthetic construct
MLIFVRMLTGKMIELEVEPSDTIENIKAKIQDHEGIPPDQQRLAFQGKSLEDGRTLSDYNILKDPKKMPLLRLR

SEQ ID NO: 508 - protein, 74 aa, synthetic construct
MLIFVRMLTGKMIELEVEPSDTIENVKAKICDHEGIPPDQQRLAFQGKSLEDGRTLSDYNILKDPKKMPLLRLR

SEQ ID NO: 509 - protein, 74 aa, synthetic construct
MLIFVRMLTGKMIELEVEPSDTIENVKAKIWDHEGIPPDQQRLAFQGKSLEDGRTLSDYNILKDPKKMPLLRLR

SEQ ID NO: 510 - protein, 74 aa, synthetic construct
MLIFVRMLTGKMIELEVEPSDTIENVKAKIFDHEGIPPDQQRLAFQGKSLEDGRTLSDYNILKDPKKMPLLRLR

SEQ ID NO: 511 - protein, 74 aa, synthetic construct
MLIFVRMLTGKMIELEVEPSDTIENVKAKIQAHEGIPPDQQRLAFQGKSLEDGRTLSDYNILKDPKKMPLLRLR

SEQ ID NO: 512 - protein, 74 aa, synthetic construct
MLIFVRMLTGKMIELEVEPSDTIENVKAKIQDSEGIPPDQQRLAFQGKSLEDGRTLSDYNILKDPKKMPLLRLR

SEQ ID NO: 513 - protein, 74 aa, synthetic construct
MLIFVRMLTGKMIELEVEPSDTIENVKAKIQDQEGIPPDQQRLAFQGKSLEDGRTLSDYNILKDPKKMPLLRLR

SEQ ID NO: 514 - protein, 74 aa, synthetic construct
MLIFVRMLTGKMIELEVEPSDTIENVKAKIQDAEGIPPDQQRLAFQGKSLEDGRTLSDYNILKDPKKMPLLRLR

SEQ ID NO: 515 - protein, 74 aa, synthetic construct
MLIFVRMLTGKMIELEVEPSDTIENVKAKIQDHEGIPLDQQRLAFQGKSLEDGRTLSDYNILKDPKKMPLLRLR

SEQ ID NO: 516 - protein, 74 aa, synthetic construct
MLIFVRMLTGKMIELEVEPSDTIENVKAKIQDHEGIPCDQQRLAFQGKSLEDGRTLSDYNILKDPKKMPLLRLR

SEQ ID NO: 517 - protein, 74 aa, synthetic construct
MLIFVRMLTGKMIELEVEPSDTIENVKAKIQDHEGIPPDEQRLAFQGKSLEDGRTLSDYNILKDPKKMPLLRLR

SEQ ID NO: 518 - protein, 74 aa, synthetic construct
MLIFVRMLTGKMIELEVEPSDTIENVKAKIQDHEGIPPDQQHLAFQGKSLEDGRTLSDYNILKDPKKMPLLRLR

SEQ ID NO: 519 - protein, 74 aa, synthetic construct
MLIFVRMLTGKMIELEVEPSDTIENVKAKIQDHEGIPPDQQFLAFQGKSLEDGRTLSDYNILKDPKKMPLLRLR

SEQ ID NO: 520 - protein, 74 aa, synthetic construct
MLIFVRMLTGKMIELEVEPSDTIENVKAKIQDHEGIPPDQQRLTFQGKSLEDGRTLSDYNILKDPKKMPLLRLR

SEQ ID NO: 521 - protein, 74 aa, synthetic construct
MLIFVRMLTGKMIELEVEPSDTIENVKAKIQDHEGIPPDQQRLAFQGTSLEDGRTLSDYNILKDPKKMPLLRLR

SEQ ID NO: 522 - protein, 74 aa, synthetic construct
MLIFVRMLTGKMIELEVEPSDTIENVKAKIQDHEGIPPDQQRLAFQGKLLEDGRTLSDYNILKDPKKMPLLRLR

SEQ ID NO: 523 - protein, 74 aa, synthetic construct
MLIFVRMLTGKMIELEVEPSDTIENVKAKIQDHEGIPPDQQRLAFQGKMLEDGRTLSDYNILKDPKKMPLLRLR

SEQ ID NO: 524 - protein, 74 aa, synthetic construct
MLIFVRMLTGKMIELEVEPSDTIENVKAKIQDHEGIPPDQQRLAFQGKSLDDGRTLSDYNILKDPKKMPLLRLR

SEQ ID NO: 525 - protein, 73 aa, synthetic construct
MLIFVRMLTGKMIELEVEPSDTIENVKAKIQDHEGIPPDQQRLAFQGKSLEDGYTLSDYNILKDPKKMPLLRL

SEQ ID NO: 526 - protein, 74 aa, synthetic construct
MLIFVRMLTGKMIELEVEPSDTIENVKAKIQDHEGIPPDQQRLAFQGKSLEDGRTLGDYNILKDPKKMPLLRLR

SEQ ID NO: 527 - protein, 74 aa, synthetic construct
MLIFVRMLTGKMIELEVEPSDTIENVKAKIQDHEGIPPDQQRLAFQGKSLEDGRTLSDYNLLKDPKKMPLLRLR

SEQ ID NO: 528 - protein, 74 aa, synthetic construct
MLIFVRMLTGKMIELEVEPSDTIENVKAKIQDHEGIPPDQQRLAFQGKSLEDGRTLSDYNILIDPKKMPLLRLR

SEQ ID NO: 529 - protein, 74 aa, synthetic construct
MLIFVRMLTGKMIELEVEPSDTIENVKAKIQDHEGIPPDQQRLAFQGKSLEDGRTLSDYNILKDHKKMPLLRLR

SEQ ID NO: 530 - protein, 74 aa, synthetic construct
MLIFVRMLTGKMIELEVEPSDTIENVKAKIQDHEGIPPDQQRLAFQGKSLEDGRTLSDYNILKDPKKMPLLRMR

SEQ ID NO: 531 - protein, 74 aa, synthetic construct
MLIFVRMLTGKMIELEVEPSDTIENVKAKIQDHEGIPPDQQRLAFQGKSLEDGRTLSDYNILKDPKKMPLLRLQ

SEQ ID NO: 532 - protein, 74 aa, synthetic construct
MLIFVKMLTGKMIELEVEPSDTIENVKAKIQDHEGIPPDQQRLAFQGKSLEDGRTLSDYNILKDPKKMLVLRLR

SEQ ID NO: 533 - protein, 74 aa, synthetic construct
MLIFVRMLTGKMIELEVYPSDTIENVKAKIQDHEGIPLDQQRLAFQGKLLEDGRTLGDYNILKDPKKMPLLRLR

SEQ ID NO: 534 - protein, 74 aa, synthetic construct
HLIFVRMLTGKMIELEVEPSDTIENVKAKIQDHEGIPPDQQRLAFQGKSLEDGRTLSDYNILKDPKKMPLLRLQ

SEQ ID NO: 535 - protein, 74 aa, synthetic construct
YLIFVRMLTGKMIELEVEPSDTIENIKAKIQDHEGIPPDQQRLAFQGKSLEDGRTLSDYNILKDPKKMPLLRMR

SEQ ID NO: 536 - protein, 74 aa, synthetic construct
MLIFVRMLTGKMIELEVEPSDTIEVVKAKIQDHEGIPPDEQRLAFQGKSLDDGRTLSDYNILKDPKKMPLLRLR

SEQ ID NO: 537 - protein, 74 aa, synthetic construct
MLIFVRMLTGKMIELEVEPSDTIENVKAKIQDHEGIPPDQQRLAFQGKSLEDGRTLSDYNLLIDPKKMPLLRLR

SEQ ID NO: 538 - protein, 74 aa, synthetic construct
MLIFVRMLTGKMIELEVMPSDTIENVKAKIQDHEGIPPDQQRLAFQGTSLDDGRTLGDYNILKDPKKMPLLRLR

SEQ ID NO: 539 - protein, 74 aa, synthetic construct
MLIFVRMLTGKMIELMVEPSDTIEVVKAKIQDHEGIPPDEQRLAFQGKLLEDGRTLSDYNILKDPKKMPLLRLR

SEQ ID NO: 540 - protein, 74 aa, synthetic construct
MLIFVRMLTGKMIELEVEPSDTIENIKAKIQDHEGIPPDQYRLAFQGKLLEDGRTLGDYNILKDPKKMPLLRLR

SEQ ID NO: 541 - protein, 74 aa, synthetic construct
MLIFVRMLTGKMIELEVEPSDTIENVKAKICDHEGIPPDQQHLAFQGKSLEDGRTLGDYNILKDPKKMPLLRLR

SEQ ID NO: 542 - protein, 74 aa, synthetic construct
MLIFVRMLTGKMIELEVMPSDTIEVVKAKIFDHEGIPPDQQHLAFQGTSLEDGYTLGDYNILKDPKKMPLLRLR

SEQ ID NO: 543 - protein, 74 aa, synthetic construct
MLIFVRMLTGKMIELEVMPSDTIEVVKAKIFDHEGIPPDQQHLTFQGTLLEDGYTLGDYNILKDPKKMPLLRLR

SEQ ID NO: 544 - protein, 74 aa, synthetic construct
MLIFVRMLTGKMIELEVMPSDTIEVVKAKIFDHEGIPLDQQHLAFQGTSLEDGYTLGDYNILKDPKKMPLLRLR

SEQ ID NO: 545 - protein, 74 aa, synthetic construct
MLIFVRMLTGKMIELEVMPSDTIEVVKAKIQDHEGIPPDQQHLAFQGTSLEDGYTLGDYNILKDPKKMPLLRLR

SEQ ID NO: 546 - protein, 74 aa, synthetic construct
YLIFVRMLTGKMIELEVEPSDTIENIKAKIQDHEGIPPDQQRLAFQGKSLEDGRTLSDYNLLIDPKKMPLLRMR

SEQ ID NO: 547 - protein, 74 aa, synthetic construct
YLIFVRMLTGKMIELEVEPSDTIEVIKAKIQDHEGIPPDEQRLAFQGKSLDDGRTLSDYNLLIDPKKMPLLRMR

SEQ ID NO: 548 - protein, 74 aa, synthetic construct
MLIFVRMLTGKMIELEVEPSDTIEVVKAKIQDHEGIPPDEQRLAFQGKSLDDGRTLSDYNLLIDPKKMPLLRLR

SEQ ID NO: 549 - protein, 74 aa, synthetic construct
MLIFVRMLTGKMIELEVMPSDTIENVKAKIQDHEGIPPDQQRLAFQGTSLDDGRTLGDYNLLIDPKKMPLLRLR

SEQ ID NO: 550 - protein, 74 aa, synthetic construct
MLIFVRMLTGKMIELMVEPSDTIEVVKAKIQDHEGIPPDEQRLAFQGKLLEDGRTLSDYNLLIDPKKMPLLRLR

SEQ ID NO: 551 - protein, 74 aa, synthetic construct
YLIFVRMLTGKMIELEVMPSDTIENIKAKIQDHEGIPPDQQRLAFQGTSLDDGRTLGDYNLLIDPKKMPLLRMR

SEQ ID NO: 552 - protein, 74 aa, synthetic construct
YLIFVRMLTGKMIELEVMPSDTIENIKAKIQDHEGIPPDQQRLAFQGTSLDDGRTLGDYNLLIDPKKMPLLRMQ

SEQ ID NO: 553 - protein, 74 aa, synthetic construct
YLIFVRMLTGKMIELEVMPSDTIEVIKAKIQDHEGIPPDEQRLAFQGTSLDDGRTLGDYNLLIDPKKMPLLRMQ

SEQ ID NO: 554 - protein, 74 aa, synthetic construct
MLIFVRMLTGKMIELEVEPSDTIENVKAKIQDHEGIPPDQQRLTFQGKLLEDGRTLSDYNLLIDPKKMPLLRLR

SEQ ID NO: 555 - protein, 74 aa, synthetic construct
MLIFVRMLTGKMIELEVEPSDTIENVKAKIQDHEGIPLDQQRLTFQGKLLEDGRTLSDYNLLIDPKKMPLLRLR

SEQ ID NO: 556 - protein, 74 aa, synthetic construct
MLIFVRMLTGKMIELEVEPSDTIENVKAKIQDHEGIPLDQQRLTFQGKLLEDGRTLSDYNLLIDPKKMPLLRLQ

SEQ ID NO: 557 - protein, 74 aa, synthetic construct
YLIFVRMLTGKMIELEVEPSDTIENIKAKIQDHEGIPLDQQRLTFQGKLLEDGRTLSDYNLLIDPKKMPLLRMQ

SEQ ID NO: 558 - protein, 74 aa, synthetic construct
MLIFVRMLTGKMIELEVEPSDTIEVVKAKIQDHEGIPLDEQRLTFQGKLLDDGRTLSDYNLLIDPKKMPLLRLQ

SEQ ID NO: 559 - protein, 74 aa, synthetic construct
YLIFVRMLTGKMIELEVEPSDTIEVIKAKIQDHEGIPLDEQRLTFQGKLLDDGRTLSDYNLLIDPKKMPLLRMQ

SEQ ID NO: 560 - protein, 74 aa, synthetic construct
MLIFVRMLTGKMIELEVMPSDTIEVVKAKIQDHEGIPPDQQHLTFQGTLLEDGYTLGDYNLLIDPKKMPLLRLR

SEQ ID NO: 561 - protein, 74 aa, synthetic construct
MLIFVRMLTGKMIELEVMPSDTIEVVKAKIQDHEGIPLDQQHLTFQGTLLEDGYTLGDYNLLIDPKKMPLLRLR

SEQ ID NO: 562 - protein, 74 aa, synthetic construct
MLIFVRMLTGKMIELEVMPSDTIEVVKAKIQDHEGIPLDQQHLTFQGTLLEDGYTLGDYNLLIDPKKMPLLRLQ

SEQ ID NO: 563 - protein, 74 aa, synthetic construct
MLIFVRMLTGKMIELEVMPSDTIEVVKAKIQDHEGIPLDEQHLTFQGTLLDDGYTLGDYNLLIDPKKMPLLRLQ

SEQ ID NO: 564 - protein, 74 aa, synthetic construct
YLIFVRMLTGKMIELEVMPSDTIEVIKAKIQDHEGIPLDEQHLTFQGTLLDDGYTLGDYNLLIDPKKMPLLRMQ

SEQ ID NO: 565 - protein, 74 aa, synthetic construct
YLIFVRMLTGKMIELEVMPSDTIEVIKAKIQDHEGIPPDEQRLAFQGTSLDDGRTLGDYNLLIDPKKMLVLRMQ

SEQ ID NO: 566 - protein, 74 aa, synthetic construct
YLIFVRMLTGKMIELEVEPSDTIENIKAKIQDHEGIPLDQQRLTFQGKLLEDGRTLSDYNLLIDPKKMLVLRMQ

SEQ ID NO: 567 - protein, 74 aa, synthetic construct
MLIFVRMLTGKMIELEVEPSDTIEVVKAKIQDHEGIPLDEQRLTFQGKLLDDGRTLSDYNLLIDPKKMLVLRLQ

SEQ ID NO: 568 - protein, 74 aa, synthetic construct
MLIFVRMLTGKMIELEVMPSDTIEVVKAKIQDHEGIPLDQQHLTFQGTLLEDGYTLGDYNLLIDPKKMLVLRLQ

SEQ ID NO: 569 - protein, 74 aa, synthetic construct
YLIFVRMLTGKMIELEVMPSDTIEVIKAKIQDHEGIPLDEQHLTFQGTLLDDGYTLGDYNLLIDPKKMLVLRMQ

SEQ ID NO: 570 - protein, 74 aa, synthetic construct
MLIFVRMLTGKMIELEVMPSDTIEVVKAKIQDHEGIPPDQQHLAFQGTSLEDGYTLGDYNLLIDPKKMLVLRLR

SEQ ID NO: 571 - protein, 74 aa, synthetic construct
YLIFVRMLTGKMIELEVEPSDTIENIKAKIQDHEGIPLDQQRLTFQGKLLEDGRTLSDYNLLIDPKKMAVLRMQ

SEQ ID NO: 572 - protein, 74 aa, synthetic construct
YLIFVRMLTGKMIELEVEPSDTIENIKAKIQDHEGIPLDQQRLTFQGKLLEDGRTLSDYNLLIDPKKMRVLRMQ

SEQ ID NO: 573 - protein, 74 aa, synthetic construct
YLIFVRMLTGKMIELEVEPSDTIENIKAKIQDHEGIPLDQQRLTFQGKLLEDGRTLSDYNLLIDPKKMNVLRMQ

SEQ ID NO: 574 - protein, 74 aa, synthetic construct
YLIFVRMLTGKMIELEVEPSDTIENIKAKIQDHEGIPLDQQRLTFQGKLLEDGRTLSDYNLLIDPKKMDVLRMQ

SEQ ID NO: 575 - protein, 74 aa, synthetic construct
YLIFVRMLTGKMIELEVEPSDTIENIKAKIQDHEGIPLDQQRLTFQGKLLEDGRTLSDYNLLIDPKKMCVLRMQ

SEQ ID NO: 576 - protein, 74 aa, synthetic construct
YLIFVRMLTGKMIELEVEPSDTIENIKAKIQDHEGIPLDQQRLTFQGKLLEDGRTLSDYNLLIDPKKMEVLRMQ

SEQ ID NO: 577 - protein, 74 aa, synthetic construct
YLIFVRMLTGKMIELEVEPSDTIENIKAKIQDHEGIPLDQQRLTFQGKLLEDGRTLSDYNLLIDPKKMQVLRMQ

SEQ ID NO: 578 - protein, 74 aa, synthetic construct
YLIFVRMLTGKMIELEVEPSDTIENIKAKIQDHEGIPLDQQRLTFQGKLLEDGRTLSDYNLLIDPKKMGVLRMQ

SEQ ID NO: 579 - protein, 74 aa, synthetic construct
YLIFVRMLTGKMIELEVEPSDTIENIKAKIQDHEGIPLDQQRLTFQGKLLEDGRTLSDYNLLIDPKKMHVLRMQ

SEQ ID NO: 580 - protein, 74 aa, synthetic construct
YLIFVRMLTGKMIELEVEPSDTIENIKAKIQDHEGIPLDQQRLTFQGKLLEDGRTLSDYNLLIDPKKMIVLRMQ

SEQ ID NO: 581 - protein, 74 aa, synthetic construct
YLIFVRMLTGKMIELEVEPSDTIENIKAKIQDHEGIPLDQQRLTFQGKLLEDGRTLSDYNLLIDPKKMKVLRMQ

SEQ ID NO: 582 - protein, 74 aa, synthetic construct
YLIFVRMLTGKMIELEVEPSDTIENIKAKIQDHEGIPLDQQRLTFQGKLLEDGRTLSDYNLLIDPKKMMVLRMQ

SEQ ID NO: 583 - protein, 74 aa, synthetic construct
YLIFVRMLTGKMIELEVEPSDTIENIKAKIQDHEGIPLDQQRLTFQGKLLEDGRTLSDYNLLIDPKKMFVLRMQ

SEQ ID NO: 584 - protein, 74 aa, synthetic construct
YLIFVRMLTGKMIELEVEPSDTIENIKAKIQDHEGIPLDQQRLTFQGKLLEDGRTLSDYNLLIDPKKMSVLRMQ

SEQ ID NO: 585 - protein, 74 aa, synthetic construct
YLIFVRMLTGKMIELEVEPSDTIENIKAKIQDHEGIPLDQQRLTFQGKLLEDGRTLSDYNLLIDPKKMTVLRMQ

SEQ ID NO: 586 - protein, 74 aa, synthetic construct
YLIFVRMLTGKMIELEVEPSDTIENIKAKIQDHEGIPLDQQRLTFQGKLLEDGRTLSDYNLLIDPKKMWVLRMQ

SEQ ID NO: 587 - protein, 74 aa, synthetic construct
YLIFVRMLTGKMIELEVEPSDTIENIKAKIQDHEGIPLDQQRLTFQGKLLEDGRTLSDYNLLIDPKKMYVLRMQ

SEQ ID NO: 588 - protein, 74 aa, synthetic construct
YLIFVRMLTGKMIELEVEPSDTIENIKAKIQDHEGIPLDQQRLTFQGKLLEDGRTLSDYNLLIDPKKMVVLRMQ

SEQ ID NO: 589 - protein, 74 aa, synthetic construct
YLIFVRMLTGKMIELEVEPSDTIENIKAKIQDHEGIPLDQQRLTFQGKLLEDGRTLSDYNLLIDPKKMLALRMQ

SEQ ID NO: 590 - protein, 74 aa, synthetic construct
YLIFVRMLTGKMIELEVEPSDTIENIKAKIQDHEGIPLDQQRLTFQGKLLEDGRTLSDYNLLIDPKKMLRLRMQ

SEQ ID NO: 591 - protein, 74 aa, synthetic construct
YLIFVRMLTGKMIELEVEPSDTIENIKAKIQDHEGIPLDQQRLTFQGKLLEDGRTLSDYNLLIDPKKMLNLRMQ

SEQ ID NO: 592 - protein, 74 aa, synthetic construct
YLIFVRMLTGKMIELEVEPSDTIENIKAKIQDHEGIPLDQQRLTFQGKLLEDGRTLSDYNLLIDPKKMLDLRMQ

SEQ ID NO: 593 - protein, 74 aa, synthetic construct
YLIFVRMLTGKMIELEVEPSDTIENIKAKIQDHEGIPLDQQRLTFQGKLLEDGRTLSDYNLLIDPKKMLCLRMQ

SEQ ID NO: 594 - protein, 74 aa, synthetic construct
YLIFVRMLTGKMIELEVEPSDTIENIKAKIQDHEGIPLDQQRLTFQGKLLEDGRTLSDYNLLIDPKKMLELRMQ

SEQ ID NO: 595 - protein, 74 aa, synthetic construct
YLIFVRMLTGKMIELEVEPSDTIENIKAKIQDHEGIPLDQQRLTFQGKLLEDGRTLSDYNLLIDPKKMLQLRMQ

SEQ ID NO: 596 - protein, 74 aa, synthetic construct
YLIFVRMLTGKMIELEVEPSDTIENIKAKIQDHEGIPLDQQRLTFQGKLLEDGRTLSDYNLLIDPKKMLGLRMQ

SEQ ID NO: 597 - protein, 74 aa, synthetic construct
YLIFVRMLTGKMIELEVEPSDTIENIKAKIQDHEGIPLDQQRLTFQGKLLEDGRTLSDYNLLIDPKKMLHLRMQ

SEQ ID NO: 598 - protein, 74 aa, synthetic construct
YLIFVRMLTGKMIELEVEPSDTIENIKAKIQDHEGIPLDQQRLTFQGKLLEDGRTLSDYNLLIDPKKMLILRMQ

SEQ ID NO: 599 - protein, 74 aa, synthetic construct
YLIFVRMLTGKMIELEVEPSDTIENIKAKIQDHEGIPLDQQRLTFQGKLLEDGRTLSDYNLLIDPKKMLKLRMQ

SEQ ID NO: 600 - protein, 74 aa, synthetic construct
YLIFVRMLTGKMIELEVEPSDTIENIKAKIQDHEGIPLDQQRLTFQGKLLEDGRTLSDYNLLIDPKKMLMLRMQ

SEQ ID NO: 601 - protein, 74 aa, synthetic construct
YLIFVRMLTGKMIELEVEPSDTIENIKAKIQDHEGIPLDQQRLTFQGKLLEDGRTLSDYNLLIDPKKMLFLRMQ

SEQ ID NO: 602 - protein, 74 aa, synthetic construct
YLIFVRMLTGKMIELEVEPSDTIENIKAKIQDHEGIPLDQQRLTFQGKLLEDGRTLSDYNLLIDPKKMLPLRMQ

SEQ ID NO: 603 - protein, 74 aa, synthetic construct
YLIFVRMLTGKMIELEVEPSDTIENIKAKIQDHEGIPLDQQRLTFQGKLLEDGRTLSDYNLLIDPKKMLSLRMQ

SEQ ID NO: 604 - protein, 74 aa, synthetic construct
YLIFVRMLTGKMIELEVEPSDTIENIKAKIQDHEGIPLDQQRLTFQGKLLEDGRTLSDYNLLIDPKKMLTLRMQ

SEQ ID NO: 605 - protein, 74 aa, synthetic construct
YLIFVRMLTGKMIELEVEPSDTIENIKAKIQDHEGIPLDQQRLTFQGKLLEDGRTLSDYNLLIDPKKMLWLRMQ

SEQ ID NO: 606 - protein, 74 aa, synthetic construct
YLIFVRMLTGKMIELEVEPSDTIENIKAKIQDHEGIPLDQQRLTFQGKLLEDGRTLSDYNLLIDPKKMLYLRMQ

SEQ ID NO: 607 - protein, 74 aa, synthetic construct
YMIFVRMLTGKMIELEVEPSDTIENIKAKIQDHEGIPLDQQRLTFQGKLLEDGRTLSDYNLPISPEKMAVLRMQ

SEQ ID NO: 608 - protein, 74 aa, synthetic construct
YMIFVRMLTGKMIELEVEPSDTIENIKAKIQDHEGIPLDQQRLTFQGKLLEDGRTLSDYNLPISPEKMGVLRMQ

SEQ ID NO: 609 - protein, 74 aa, synthetic construct
YMIFVRMLTGKMIELEVEPSDTIENIKAKIQDHEGIPLDQQRLTFQGKLLEDGRTLSDYNLPISPEKMLMLRMQ

SEQ ID NO: 610 - protein, 74 aa, synthetic construct
YMIFVRMLTGKMIELEVEPSDTIENIKAKIQDHEGIPLDQQRLTFQGKLLEDGRTLSDYNLPISPEKMAMLRMQ

SEQ ID NO: 611 - protein, 74 aa, synthetic construct
YMIFVRMLTGKMIELEVEPSDTIENIKAKIQDHEGIPLDQQRLTFQGKLLEDGRTLSDYNLPISPEKMAFLRMQ

SEQ ID NO: 612 - protein, 74 aa, synthetic construct
YMIFVRMLTGKMIELEVEPSDTIENIKAKIQDHEGIPLDQQRLTFQGKLLEDGRTLSDYNLPISPEKMACLRMQ

SEQ ID NO: 613 - protein, 74 aa, synthetic construct
YMIFVRMLTGKMIELEVEPSDTIENIKAKIQDHEGIPLDQQRLTFQGKLLEDGRTLSDYNLPISPEKMGMLRMQ

SEQ ID NO: 614 - protein, 74 aa, synthetic construct
YMIFVRMLTGKMIELEVEPSDTIENIKAKIQDHEGIPLDQQRLTFQGKLLEDGRTLSDYNLPISPEKMGFLRMQ

SEQ ID NO: 615 - protein, 74 aa, synthetic construct
YMIFVRMLTGKMIELEVEPSDTIENIKAKIQDHEGIPLDQQRLTFQGKLLEDGRTLSDYNLPISPEKMGCLRMQ

SEQ ID NO: 616 - protein, 74 aa, synthetic construct
YMIFVRMLTGKMIELEVEPSDTIENIKAKIQDHEGIPLDQQRLTFQGKLLEDGRTLSDYNLPISPEKMCMLRMQ

SEQ ID NO: 617 - protein, 74 aa, synthetic construct
YMIFVRMLTGKMIELEVEPSDTIENIKAKIQDHEGIPLDQQRLTFQGKLLEDGRTLSDYNLPISPEKMMMLRMQ

SEQ ID NO: 618 - protein, 74 aa, synthetic construct
YMIFVRMLTGKMIELEVEPSDTIENIKAKIQDHEGIPLDQQRLTFQGKLLEDGRTLSDYNLPISPEKMFMLRMQ

SEQ ID NO: 619 - protein, 74 aa, synthetic construct
YMIFVRMLTGKMIELEVEPSDTIENIKAKIQDHEGIPLDQQRLTFQGKLLEDGRTLSDYNLAISPEKMAVLRMQ

SEQ ID NO: 620 - protein, 74 aa, synthetic construct
YMIFVRMLTGKMIELEVEPSDTIENIKAKIQDHEGIPLDQQRLTFQGKLLEDGRTLSDYNLRISPEKMAVLRMQ

SEQ ID NO: 621 - protein, 74 aa, synthetic construct
YMIFVRMLTGKMIELEVEPSDTIENIKAKIQDHEGIPLDQQRLTFQGKLLEDGRTLSDYNLNISPEKMAVLRMQ

SEQ ID NO: 622 - protein, 74 aa, synthetic construct
YMIFVRMLTGKMIELEVEPSDTIENIKAKIQDHEGIPLDQQRLTFQGKLLEDGRTLSDYNLDISPEKMAVLRMQ

SEQ ID NO: 623 - protein, 74 aa, synthetic construct
YMIFVRMLTGKMIELEVEPSDTIENIKAKIQDHEGIPLDQQRLTFQGKLLEDGRTLSDYNLCISPEKMAVLRMQ

SEQ ID NO: 624 - protein, 74 aa, synthetic construct
YMIFVRMLTGKMIELEVEPSDTIENIKAKIQDHEGIPLDQQRLTFQGKLLEDGRTLSDYNLEISPEKMAVLRMQ

SEQ ID NO: 625 - protein, 74 aa, synthetic construct
YMIFVRMLTGKMIELEVEPSDTIENIKAKIQDHEGIPLDQQRLTFQGKLLEDGRTLSDYNLQISPEKMAVLRMQ

SEQ ID NO: 626 - protein, 74 aa, synthetic construct
YMIFVRMLTGKMIELEVEPSDTIENIKAKIQDHEGIPLDQQRLTFQGKLLEDGRTLSDYNLGISPEKMAVLRMQ

SEQ ID NO: 627 - protein, 74 aa, synthetic construct
YMIFVRMLTGKMIELEVEPSDTIENIKAKIQDHEGIPLDQQRLTFQGKLLEDGRTLSDYNLHISPEKMAVLRMQ

SEQ ID NO: 628 - protein, 74 aa, synthetic construct
YMIFVRMLTGKMIELEVEPSDTIENIKAKIQDHEGIPLDQQRLTFQGKLLEDGRTLSDYNLIISPEKMAVLRMQ

SEQ ID NO: 629 - protein, 74 aa, synthetic construct
YMIFVRMLTGKMIELEVEPSDTIENIKAKIQDHEGIPLDQQRLTFQGKLLEDGRTLSDYNLKISPEKMAVLRMQ

SEQ ID NO: 630 - protein, 74 aa, synthetic construct
YMIFVRMLTGKMIELEVEPSDTIENIKAKIQDHEGIPLDQQRLTFQGKLLEDGRTLSDYNLMISPEKMAVLRMQ

SEQ ID NO: 631 - protein, 74 aa, synthetic construct
YMIFVRMLTGKMIELEVEPSDTIENIKAKIQDHEGIPLDQQRLTFQGKLLEDGRTLSDYNLFISPEKMAVLRMQ

SEQ ID NO: 632 - protein, 74 aa, synthetic construct
YMIFVRMLTGKMIELEVEPSDTIENIKAKIQDHEGIPLDQQRLTFQGKLLEDGRTLSDYNLSISPEKMAVLRMQ

SEQ ID NO: 633 - protein, 74 aa, synthetic construct
YMIFVRMLTGKMIELEVEPSDTIENIKAKIQDHEGIPLDQQRLTFQGKLLEDGRTLSDYNLTISPEKMAVLRMQ

SEQ ID NO: 634 - protein, 74 aa, synthetic construct
YMIFVRMLTGKMIELEVEPSDTIENIKAKIQDHEGIPLDQQRLTFQGKLLEDGRTLSDYNLWISPEKMAVLRMQ

SEQ ID NO: 635 - protein, 74 aa, synthetic construct
YMIFVRMLTGKMIELEVEPSDTIENIKAKIQDHEGIPLDQQRLTFQGKLLEDGRTLSDYNLYISPEKMAVLRMQ

SEQ ID NO: 636 - protein, 74 aa, synthetic construct
YMIFVRMLTGKMIELEVEPSDTIENIKAKIQDHEGIPLDQQRLTFQGKLLEDGRTLSDYNLVISPEKMAVLRMQ

SEQ ID NO: 637 - protein, 74 aa, synthetic construct
YMIFVRMLTGKMIELEVEPSDTIENIKAKIQDHEGIPLDQQRLTFQGKLLEDGRTLSDYNLPISPEKMCFLRMQ

SEQ ID NO: 638 - protein, 74 aa, synthetic construct
YMIFVRMLTGKMIELEVEPSDTIENIKAKIQDHEGIPLDQQRLTFQGKLLEDGRTLSDYNLPISPEKMMFLRMQ

SEQ ID NO: 639 - protein, 74 aa, synthetic construct
YMIFVRMLTGKMIELEVEPSDTIENIKAKIQDHEGIPLDQQRLTFQGKLLEDGRTLSDYNLPISPEKMFFLRMQ

SEQ ID NO: 640 - protein, 74 aa, synthetic construct
YMIFVRMLTGKMIELEVEPSDTIENIKAKIQDHEGIPLDQQRLTFQGKLLEDGRTLSDYNLPISPEKMCCLRMQ

SEQ ID NO: 641 - protein, 74 aa, synthetic construct
YMIFVRMLTGKMIELEVEPSDTIENIKAKIQDHEGIPLDQQRLTFQGKLLEDGRTLSDYNLPISPEKMMCLRMQ

SEQ ID NO: 642 - protein, 74 aa, synthetic construct
YMIFVRMLTGKMIELEVEPSDTIENIKAKIQDHEGIPLDQQRLTFQGKLLEDGRTLSDYNLPISPEKMFCLRMQ

SEQ ID NO: 643 - protein, 74 aa, synthetic construct
YMIFVRMLTGKMIELEVEPSDTIENIKAKIQDHEGIPLDQQRLTFQGKLLEDGRTLSDYNLAISPEKMAMLRMQ

SEQ ID NO: 644 - protein, 74 aa, synthetic construct
YMIFVRMLTGKMIELEVEPSDTIENIKAKIQDHEGIPLDQQRLTFQGKLLEDGRTLSDYNLCISPEKMAMLRMQ

SEQ ID NO: 645 - protein, 74 aa, synthetic construct
YMIFVRMLTGKMIELEVEPSDTIENIKAKIQDHEGIPLDQQRLTFQGKLLEDGRTLSDYNLTISPEKMAMLRMQ

SEQ ID NO: 646 - protein, 74 aa, synthetic construct
YMIFVRMLTGKMIELEVEPSDTIENIKAKIQDHEGIPLDQQRLTFQGKLLEDGRTLSDYNLVISPEKMAMLRMQ

SEQ ID NO: 647 - protein, 74 aa, synthetic construct
YAIFVRMLTGKMIELEVEPSDTIENIKAKIQDHEGIPLDQQRLTFQGKLLEDGRTLSDYNLTISPEKMAVLRMQ

SEQ ID NO: 648 - protein, 74 aa, synthetic construct
YRIFVRMLTGKMIELEVEPSDTIENIKAKIQDHEGIPLDQQRLTFQGKLLEDGRTLSDYNLTISPEKMAVLRMQ

SEQ ID NO: 649 - protein, 74 aa, synthetic construct
YNIFVRMLTGKMIELEVEPSDTIENIKAKIQDHEGIPLDQQRLTFQGKLLEDGRTLSDYNLTISPEKMAVLRMQ

SEQ ID NO: 650 - protein, 74 aa, synthetic construct
YDIFVRMLTGKMIELEVEPSDTIENIKAKIQDHEGIPLDQQRLTFQGKLLEDGRTLSDYNLTISPEKMAVLRMQ

SEQ ID NO: 651 - protein, 74 aa, synthetic construct
YCIFVRMLTGKMIELEVEPSDTIENIKAKIQDHEGIPLDQQRLTFQGKLLEDGRTLSDYNLTISPEKMAVLRMQ

SEQ ID NO: 652 - protein, 74 aa, synthetic construct
YEIFVRMLTGKMIELEVEPSDTIENIKAKIQDHEGIPLDQQRLTFQGKLLEDGRTLSDYNLTISPEKMAVLRMQ

SEQ ID NO: 653 - protein, 74 aa, synthetic construct
YQIFVRMLTGKMIELEVEPSDTIENIKAKIQDHEGIPLDQQRLTFQGKLLEDGRTLSDYNLTISPEKMAVLRMQ

SEQ ID NO: 654 - protein, 74 aa, synthetic construct
YGIFVRMLTGKMIELEVEPSDTIENIKAKIQDHEGIPLDQQRLTFQGKLLEDGRTLSDYNLTISPEKMAVLRMQ

SEQ ID NO: 655 - protein, 74 aa, synthetic construct
YHIFVRMLTGKMIELEVEPSDTIENIKAKIQDHEGIPLDQQRLTFQGKLLEDGRTLSDYNLTISPEKMAVLRMQ

SEQ ID NO: 656 - protein, 74 aa, synthetic construct
YIIFVRMLTGKMIELEVEPSDTIENIKAKIQDHEGIPLDQQRLTFQGKLLEDGRTLSDYNLTISPEKMAVLRMQ

SEQ ID NO: 657 - protein, 74 aa, synthetic construct
YKIFVRMLTGKMIELEVEPSDTIENIKAKIQDHEGIPLDQQRLTFQGKLLEDGRTLSDYNLTISPEKMAVLRMQ

SEQ ID NO: 658 - protein, 74 aa, synthetic construct
YLIFVRMLTGKMIELEVEPSDTIENIKAKIQDHEGIPLDQQRLTFQGKLLEDGRTLSDYNLTISPEKMAVLRMQ

SEQ ID NO: 659 - protein, 74 aa, synthetic construct
YFIFVRMLTGKMIELEVEPSDTIENIKAKIQDHEGIPLDQQRLTFQGKLLEDGRTLSDYNLTISPEKMAVLRMQ

SEQ ID NO: 660 - protein, 74 aa, synthetic construct
YSIFVRMLTGKMIELEVEPSDTIENIKAKIQDHEGIPLDQQRLTFQGKLLEDGRTLSDYNLTISPEKMAVLRMQ

SEQ ID NO: 661 - protein, 74 aa, synthetic construct
YTIFVRMLTGKMIELEVEPSDTIENIKAKIQDHEGIPLDQQRLTFQGKLLEDGRTLSDYNLTISPEKMAVLRMQ

SEQ ID NO: 662 - protein, 74 aa, synthetic construct
YWIFVRMLTGKMIELEVEPSDTIENIKAKIQDHEGIPLDQQRLTFQGKLLEDGRTLSDYNLTISPEKMAVLRMQ

SEQ ID NO: 663 - protein, 74 aa, synthetic construct
YYIFVRMLTGKMIELEVEPSDTIENIKAKIQDHEGIPLDQQRLTFQGKLLEDGRTLSDYNLTISPEKMAVLRMQ

SEQ ID NO: 664 - protein, 74 aa, synthetic construct
YVIFVRMLTGKMIELEVEPSDTIENIKAKIQDHEGIPLDQQRLTFQGKLLEDGRTLSDYNLTISPEKMAVLRMQ

SEQ ID NO: 665 - protein, 74 aa, synthetic construct
YPIFVRMLTGKMIELEVEPSDTIENIKAKIQDHEGIPLDQQRLTFQGKLLEDGRTLSDYNLTISPEKMAVLRMQ

SEQ ID NO: 666 - DNA, 228 bp, synthetic construct
  source 1..228: mol_type = other DNA
atgcagattttcgtgaaaacccttacggggaagaccatcaccctcgaggttgaaccctcggatacgatagaaaatgtaaaggccaagatccaggataaggaaggaattcctcctgatcagcagagactgatctttgctggcaagcagctggaagatggacgtactttgtctgactacaatattcaaaaggagtctactcttcatcttgtgttgagacttcgtggtggt

SEQ ID NO: 667 - DNA, 222 bp, synthetic construct
  source 1..222: mol_type = other DNA
atgttgattttcgtaaagacgttgactggaaagactatcactttggaagtggagccttccgatactatcgagaatgttaaggccaaaatccaagataaggaagggattcctccagatcaacaacgccttgcttttgccgggaagagcctggaggacggtcgcacactgtctgactataacattcttaaagattctaaattgcatccactgctgcgcttgcgt

SEQ ID NO: 668 - DNA, 222 bp, synthetic construct
  source 1..222: mol_type = other DNA
atgttgattttcgtaaagacgttgactggaaagactatcactttggaagtggagccttccgatactatcgagaatgttaaggccaaaatccaagataaggaagggattcctccagatcaacaacgccttgcttttgccgggaagagcctggaggacggtcgcacactgtctgactataacattcttaaagattctaaattgcatctggttctgcgcttgcgt

SEQ ID NO: 669 - DNA, 222 bp, synthetic construct
  source 1..222: mol_type = other DNA
atgttgattttcgtacgcacgttgactggaaagactatcactttggaagtggagccttccgatactatcgagaatgttaaggccaaaatccaagataaggaagggattcctccagatcaacaacgccttgcttttgccgggaagagcctggaggacggtcgcacactgtctgactataacattcttaaagattctaaattgcatccactgctgcgcttgcgt

SEQ ID NO: 670 - DNA, 222 bp, synthetic construct
  source 1..222: mol_type = other DNA
atgttgattttcgtaaagacgttgactggaaagactatcgagttggaagtggagccttccgatactatcgagaatgttaaggccaaaatccaagataaggaagggattcctccagatcaacaacgccttgcttttgccgggaagagcctggaggacggtcgcacactgtctgactataacattcttaaagattctaaattgcatccactgctgcgcttgcgt

SEQ ID NO: 671 - DNA, 222 bp, synthetic construct
  source 1..222: mol_type = other DNA
atgttgattttcgtaaagacgttgactggaaagactatcactttggaagtggagccttccgatactatcgagaatgttaaggccaaaatccaagatgccgaagggattcctccagatcaacaacgccttgcttttgccgggaagagcctggaggacggtcgcacactgtctgactataacattcttaaagattctaaattgcatccactgctgcgcttgcgt

SEQ ID NO: 672 - DNA, 222 bp, synthetic construct
  source 1..222: mol_type = other DNA
atgttgattttcgtaaagacgttgactggaaagactatcactttggaagtggagccttccgatactatcgagaatgttaaggccaaaatccaagataaggaagggattcctccagatcaacaacgccttgcttttcaagggaagagcctggaggacggtcgcacactgtctgactataacattcttaaagattctaaattgcatccactgctgcgcttgcgt

SEQ ID NO: 673 - DNA, 222 bp, synthetic construct
  source 1..222: mol_type = other DNA
atgttgattttcgtaaagacgttgactggaaagactatcactttggaagtggagccttccgatactatcgagaatgttaaggccaaaatccaagataaggaagggattcctccagatcaacaacgccttgcttttgccgggaagagcctggaggacggtcgcacactgtctgactataacattcttattgattctaaattgcatccactgctgcgcttgcgt

SEQ ID NO: 674 - DNA, 222 bp, synthetic construct
  source 1..222: mol_type = other DNA
atgttgattttcgtaaagacgttgactggaaagactatcactttggaagtggagccttccgatactatcgagaatgttaaggccaaaatccaagataaggaagggattcctccagatcaacaacgccttgcttttgccgggaagagcctggaggacggtcgcacactgtctgactataacattcttaaagatcctaaattgcatccactgctgcgcttgcgt

SEQ ID NO: 675 - DNA, 222 bp, synthetic construct
  source 1..222: mol_type = other DNA
catttgattttcgtaaagacgttgactggaaagactatcactttggaagtggagccttccgatactatcgagaatgttaaggccaaaatccaagataaggaagggattcctccagatcaacaacgccttgcttttgccgggaagagcctggaggacggtcgcacactgtctgactataacattcttaaagattctaaattgcatccactgctgcgcttgcgt

SEQ ID NO: 676 - DNA, 222 bp, synthetic construct; identical to SEQ ID NO: 669
  source 1..222: mol_type = other DNA

SEQ ID NO: 677 - DNA, 222 bp, synthetic construct
  source 1..222: mol_type = other DNA
atgttgattttcgtaaagatgttgactggaaagactatcactttggaagtggagccttccgatactatcgagaatgttaaggccaaaatccaagataaggaagggattcctccagatcaacaacgccttgcttttgccgggaagagcctggaggacggtcgcacactgtctgactataacattcttaaagattctaaattgcatccactgctgcgcttgcgt

SEQ ID NO: 678 - DNA, 222 bp, synthetic construct
  source 1..222: mol_type = other DNA
atgttgattttcgtaaagacgttgactggaaagatgatcactttggaagtggagccttccgatactatcgagaatgttaaggccaaaatccaagataaggaagggattcctccagatcaacaacgccttgcttttgccgggaagagcctggaggacggtcgcacactgtctgactataacattcttaaagattctaaattgcatccactgctgcgcttgcgt

SEQ ID NO: 679 - DNA, 222 bp, synthetic construct; identical to SEQ ID NO: 670
  source 1..222: mol_type = other DNA

SEQ ID NO: 680 - DNA, 222 bp, synthetic construct
  source 1..222: mol_type = other DNA
atgttgattttcgtaaagacgttgactggaaagactatcactttgatggtggagccttccgatactatcgagaatgttaaggccaaaatccaagataaggaagggattcctccagatcaacaacgccttgcttttgccgggaagagcctggaggacggtcgcacactgtctgactataacattcttaaagattctaaattgcatccactgctgcgcttgcgt

SEQ ID NO: 681 - DNA, 222 bp, synthetic construct
  source 1..222: mol_type = other DNA
atgttgattttcgtaaagacgttgactggaaagactatcactttggaagtgatgccttccgatactatcgagaatgttaaggccaaaatccaagataaggaagggattcctccagatcaacaacgccttgcttttgccgggaagagcctggaggacggtcgcacactgtctgactataacattcttaaagattctaaattgcatccactgctgcgcttgcgt

SEQ ID NO: 682 - DNA, 222 bp, synthetic construct
  source 1..222: mol_type = other DNA
atgttgattttcgtaaagacgttgactggaaagactatcactttggaagtggagccttccgatactatcgaggtagttaaggccaaaatccaagataaggaagggattcctccagatcaacaacgccttgcttttgccgggaagagcctggaggacggtcgcacactgtctgactataacattcttaaagattctaaattgcatccactgctgcgcttgcgt

SEQ ID NO: 683 - DNA, 222 bp, synthetic construct
  source 1..222: mol_type = other DNA
atgttgattttcgtaaagacgttgactggaaagactatcactttggaagtggagccttccgatactatcgagaatattaaggccaaaatccaagataaggaagggattcctccagatcaacaacgccttgcttttgccgggaagagcctggaggacggtcgcacactgtctgactataacattcttaaagattctaaattgcatccactgctgcgcttgcgt

SEQ ID NO: 684 - DNA, 222 bp, synthetic construct
  source 1..222: mol_type = other DNA
atgttgattttcgtaaagacgttgactggaaagactatcactttggaagtggagccttccgatactatcgagaatgttaaggccaaaatctgggataaggaagggattcctccagatcaacaacgccttgcttttgccgggaagagcctggaggacggtcgcacactgtctgactataacattcttaaagattctaaattgcatccactgctgcgcttgcgt

SEQ ID NO: 685 - DNA, 222 bp, synthetic construct
  source 1..222: mol_type = other DNA
atgttgattttcgtaaagacgttgactggaaagactatcactttggaagtggagccttccgatactatcgagaatgttaaggccaaaatctgcgataaggaagggattcctccagatcaacaacgccttgcttttgccgggaagagcctggaggacggtcgcacactgtctgactataacattcttaaagattctaaattgcatccactgctgcgcttgcgt

SEQ ID NO: 686 - DNA, 222 bp, synthetic construct
  source 1..222: mol_type = other DNA
atgttgattttcgtaaagacgttgactggaaagactatcactttggaagtggagccttccgatactatcgagaatgttaaggccaaaatcttcgataaggaagggattcctccagatcaacaacgccttgcttttgccgggaagagcctggaggacggtcgcacactgtctgactataacattcttaaagattctaaattgcatccactgctgcgcttgcgt

SEQ ID NO: 687 - DNA, 222 bp, synthetic construct
  source 1..222: mol_type = other DNA
atgttgattttcgtaaagacgttgactggaaagactatcactttggaagtggagccttccgatactatcgagaatgttaaggccaaaatccaagattctgaagggattcctccagatcaacaacgccttgcttttgccgggaagagcctggaggacggtcgcacactgtctgactataacattcttaaagattctaaattgcatccactgctgcgcttgcgt

SEQ ID NO: 688 - DNA, 222 bp, synthetic construct
  source 1..222: mol_type = other DNA
atgttgattttcgtaaagacgttgactggaaagactatcactttggaagtggagccttccgatactatcgagaatgttaaggccaaaatccaagatcatgaagggattcctccagatcaacaacgccttgcttttgccgggaagagcctggaggacggtcgcacactgtctgactataacattcttaaagattctaaattgcatccactgctgcgcttgcgt

SEQ ID NO: 689 - DNA, 222 bp, synthetic construct; identical to SEQ ID NO: 671
  source 1..222: mol_type = other DNA

SEQ ID NO: 690 - DNA, 222 bp, synthetic construct
  source 1..222: mol_type = other DNA
atgttgattttcgtaaagacgttgactggaaagactatcactttggaagtggagccttccgatactatcgagaatgttaaggccaaaatccaagataaggaagggattcctttggatcaacaacgccttgcttttgccgggaagagcctggaggacggtcgcacactgtctgactataacattcttaaagattctaaattgcatccactgctgcgcttgcgt

SEQ ID NO: 691 - DNA, 222 bp, synthetic construct
  source 1..222: mol_type = other DNA
atgttgattttcgtaaagacgttgactggaaagactatcactttggaagtggagccttccgatactatcgagaatgttaaggccaaaatccaagataaggaagggattcctccagatcaacaatggcttgcttttgccgggaagagcctggaggacggtcgcacactgtctgactataacattcttaaagattctaaattgcatccactgctgcgcttgcgt

SEQ ID NO: 692 - DNA, 222 bp, synthetic construct
  source 1..222: mol_type = other DNA
atgttgattttcgtaaagacgttgactggaaagactatcactttggaagtggagccttccgatactatcgagaatgttaaggccaaaatccaagataaggaagggattcctccagatcaacaacgccttacttttgccgggaagagcctggaggacggtcgcacactgtctgactataacattcttaaagattctaaattgcatccactgctgcgcttgcgt

SEQ ID NO: 693 - DNA, 222 bp, synthetic construct; identical to SEQ ID NO: 672
  source 1..222: mol_type = other DNA

SEQ ID NO: 694 - DNA, 222 bp, synthetic construct
  source 1..222: mol_type = other DNA
atgttgattttcgtaaagacgttgactggaaagactatcactttggaagtggagccttccgatactatcgagaatgttaaggccaaaatccaagataaggaagggattcctccagatcaacaacgccttgcttttgccgggactagcctggaggacggtcgcacactgtctgactataacattcttaaagattctaaattgcatccactgctgcgcttgcgt

SEQ ID NO: 695 - DNA, 222 bp, synthetic construct
  source 1..222: mol_type = other DNA
atgttgattttcgtaaagacgttgactggaaagactatcactttggaagtggagccttccgatactatcgagaatgttaaggccaaaatccaagataaggaagggattcctccagatcaacaacgccttgcttttgccgggaagagcctggaggacggttatacactgtctgactataacattcttaaagattctaaattgcatccactgctgcgcttgcgt

SEQ ID NO: 696 - DNA, 222 bp, synthetic construct
  source 1..222: mol_type = other DNA
atgttgattttcgtaaagacgttgactggaaagactatcactttggaagtggagccttccgatactatcgagaatgttaaggccaaaatccaagataaggaagggattcctccagatcaacaacgccttgcttttgccgggaagagcctggaggacggtcgcacactgggggactataacattcttaaagattctaaattgcatccactgctgcgcttgcgt

SEQ ID NO: 697 - DNA, 222 bp, synthetic construct; identical to SEQ ID NO: 673
  source 1..222: mol_type = other DNA

SEQ ID NO: 698 - DNA, 222 bp, synthetic construct; identical to SEQ ID NO: 674
  source 1..222: mol_type = other DNA

SEQ ID NO: 699 - DNA, 222 bp, synthetic construct
  source 1..222: mol_type = other DNA
atgttgattttcgtacgcatgttgactggaaagatgatcgagttggaagtggagccttccgatactatcgagaatgttaaggccaaaatccaagatcatgaagggattcctccagatcaacaacgccttgcttttcaagggaagagcctggaggacggtcgcacactgtctgactataacattcttaaagatcctaaaaagatgccactgctgcgcttgcgt

SEQ ID NO: 700 - DNA, 222 bp, synthetic construct
  source 1..222: mol_type = other DNA
atgttgattttcgtacgcacgttgactggaaagactatcactttggaagtggagccttccgatactatcgagaatgttaaggccaaaatccaagatcatgaagggattcctccagatcaacaacgccttgcttttcaagggaagagcctggaggacggtcgcacactgtctgactataacattcttaaagatcctaaattgcatccactgctgcgcttgcgt

SEQ ID NO: 701 - DNA, 222 bp, synthetic construct
  source 1..222: mol_type = other DNA
atgttgattttcgtaaagatgttgactggaaagactatcgagttggaagtggagccttccgatactatcgagaatgttaaggccaaaatccaagataaggaagggattcctccagatcaacaacgccttgcttttcaagggaagagcctggaggacggtcgcacactgtctgactataacattcttaaagattctaaaaagcatccactgctgcgcttgcgt

SEQ ID NO: 702 - DNA, 222 bp, synthetic construct
  source 1..222: mol_type = other DNA
atgttgattttcgtaaagacgttgactggaaagatgatcactttggaagtggagccttccgatactatcgagaatgttaaggccaaaatccaagatcatgaagggattcctccagatcaacaacgccttgcttttcaagggaagagcctggaggacggtcgcacactgtctgactataacattcttaaagattctaaattgatgccactgctgcgcttgcgt

SEQ ID NO: 703 - DNA, 222 bp, synthetic construct
  source 1..222: mol_type = other DNA
atgttgattttcgtaaagatgttgactggaaagatgatcgagttggaagtggagccttccgatactatcgagaatgttaaggccaaaatccaagatcatgaagggattcctccagatcaacaacgccttgcttttcaagggaagagcctggaggacggtcgcacactgtctgactataacattcttaaagatcctaaaaagatgccactgctgcgcttgcgt

SEQ ID NO: 704 - DNA, 222 bp, synthetic construct
  source 1..222: mol_type = other DNA
atgttgattttcgtacgcacgttgactggaaagatgatcgagttggaagtggagccttccgatactatcgagaatgttaaggccaaaatccaagatcatgaagggattcctccagatcaacaacgccttgcttttcaagggaagagcctggaggacggtcgcacactgtctgactataacattcttaaagatcctaaaaagatgccactgctgcgcttgcgt

SEQ ID NO: 705 - DNA, 222 bp, synthetic construct
  source 1..222: mol_type = other DNA
atgttgattttcgtacgcatgttgactggaaagactatcgagttggaagtggagccttccgatactatcgagaatgttaaggccaaaatccaagatcatgaagggattcctccagatcaacaacgccttgcttttcaagggaagagcctggaggacggtcgcacactgtctgactataacattcttaaagatcctaaaaagatgccactgctgcgcttgcgt

SEQ ID NO: 706 - DNA, 222 bp, synthetic construct
  source 1..222: mol_type = other DNA
atgttgattttcgtacgcatgttgactggaaagatgatcactttggaagtggagccttccgatactatcgagaatgttaaggccaaaatccaagatcatgaagggattcctccagatcaacaacgccttgcttttcaagggaagagcctggaggacggtcgcacactgtctgactataacattcttaaagatcctaaaaagatgccactgctgcgcttgcgt

SEQ ID NO: 707 - DNA, 222 bp, synthetic construct
  source 1..222: mol_type = other DNA
atgttgattttcgtacgcatgttgactggaaagatgatcgagttggaagtggagccttccgatactatcgagaatgttaaggccaaaatccaagataaggaagggattcctccagatcaacaacgccttgcttttcaagggaagagcctggaggacggtcgcacactgtctgactataacattcttaaagatcctaaaaagatgccactgctgcgcttgcgt

SEQ ID NO: 708 - DNA, 222 bp, synthetic construct
  source 1..222: mol_type = other DNA
atgttgattttcgtacgcatgttgactggaaagatgatcgagttggaagtggagccttccgatactatcgagaatgttaaggccaaaatccaagatcatgaagggattcctccagatcaacaacgccttgcttttgccgggaagagcctggaggacggtcgcacactgtctgactataacattcttaaagatcctaaaaagatgccactgctgcgcttgcgt

SEQ ID NO: 709 - DNA, 222 bp, synthetic construct
  source 1..222: mol_type = other DNA
atgttgattttcgtacgcatgttgactggaaagatgatcgagttggaagtggagccttccgatactatcgagaatgttaaggccaaaatccaagatcatgaagggattcctccagatcaacaacgccttgcttttcaagggaagagcctggaggacggtcgcacactgtctgactataacattcttaaagattctaaaaagatgccactgctgcgcttgcgt

SEQ ID NO: 710 - DNA, 222 bp, synthetic construct
  source 1..222: mol_type = other DNA
atgttgattttcgtacgcatgttgactggaaagatgatcgagttggaagtggagccttccgatactatcgagaatgttaaggccaaaatccaagatcatgaagggattcctccagatcaacaacgccttgcttttcaagggaagagcctggaggacggtcgcacactgtctgactataacattcttaaagatcctaaattgatgccactgctgcgcttgcgt

SEQ ID NO: 711 - DNA, 222 bp, synthetic construct
  source 1..222: mol_type = other DNA
atgttgattttcgtacgcatgttgactggaaagatgatcgagttggaagtggagccttccgatactatcgagaatgttaaggccaaaatccaagatcatgaagggattcctccagatcaacaacgccttgcttttcaagggaagagcctggaggacggtcgcacactgtctgactataacattcttaaagatcctaaaaagcatccactgctgcgcttgcgt

SEQ ID NO: 712 - DNA, 222 bp, synthetic construct
  source 1..222: mol_type = other DNA
catttgattttcgtacgcatgttgactggaaagatgatcgagttggaagtggagccttccgatactatcgagaatgttaaggccaaaatccaagatcatgaagggattcctccagatcaacaacgccttgcttttcaagggaagagcctggaggacggtcgcacactgtctgactataacattcttaaagatcctaaaaagatgccactgctgcgcttgcgt

SEQ ID NO: 713 - DNA, 222 bp, synthetic construct
  source 1..222: mol_type = other DNA
tatttgattttcgtacgcatgttgactggaaagatgatcgagttggaagtggagccttccgatactatcgagaatgttaaggccaaaatccaagatcatgaagggattcctccagatcaacaacgccttgcttttcaagggaagagcctggaggacggtcgcacactgtctgactataacattcttaaagatcctaaaaagatgccactgctgcgcttgcgt

SEQ ID NO: 714 - DNA, 222 bp, synthetic construct
  source 1..222: mol_type = other DNA
atgttgattttcgtacgcatgttgactggaaagatgatccatttggaagtggagccttccgatactatcgagaatgttaaggccaaaatccaagatcatgaagggattcctccagatcaacaacgccttgcttttcaagggaagagcctggaggacggtcgcacactgtctgactataacattcttaaagatcctaaaaagatgccactgctgcgcttgcgt

SEQ ID NO: 715 - DNA, 222 bp, synthetic construct
  source 1..222: mol_type = other DNA
atgttgattttcgtacgcatgttgactggaaagatgatcgatttggaagtggagccttccgatactatcgagaatgttaaggccaaaatccaagatcatgaagggattcctccagatcaacaacgccttgcttttcaagggaagagcctggaggacggtcgcacactgtctgactataacattcttaaagatcctaaaaagatgccactgctgcgcttgcgt

SEQ ID NO: 716 - DNA, 222 bp, synthetic construct
  source 1..222: mol_type = other DNA
atgttgattttcgtacgcatgttgactggaaagatgatcgagttgatggtggagccttccgatactatcgagaatgttaaggccaaaatccaagatcatgaagggattcctccagatcaacaacgccttgcttttcaagggaagagcctggaggacggtcgcacactgtctgactataacattcttaaagatcctaaaaagatgccactgctgcgcttgcgt

SEQ ID NO: 717 - DNA, 222 bp, synthetic construct
  source 1..222: mol_type = other DNA
atgttgattttcgtacgcatgttgactggaaagatgatcgagttgactgtggagccttccgatactatcgagaatgttaaggccaaaatccaagatcatgaagggattcctccagatcaacaacgccttgcttttcaagggaagagcctggaggacggtcgcacactgtctgactataacattcttaaagatcctaaaaagatgccactgctgcgcttgcgt

SEQ ID NO: 718 - DNA, 222 bp, synthetic construct
  source 1..222: mol_type = other DNA
atgttgattttcgtacgcatgttgactggaaagatgatcgagttggaagtgatgccttccgatactatcgagaatgttaaggccaaaatccaagatcatgaagggattcctccagatcaacaacgccttgcttttcaagggaagagcctggaggacggtcgcacactgtctgactataacattcttaaagatcctaaaaagatgccactgctgcgcttgcgt

SEQ ID NO: 719 - DNA, 222 bp, synthetic construct
  source 1..222: mol_type = other DNA
atgttgattttcgtacgcatgttgactggaaagatgatcgagttggaagtgtatccttccgatactatcgagaatgttaaggccaaaatccaagatcatgaagggattcctccagatcaacaacgccttgcttttcaagggaagagcctggaggacggtcgcacactgtctgactataacattcttaaagatcctaaaaagatgccactgctgcgcttgcgt

SEQ ID NO: 720 - DNA, 222 bp, synthetic construct
  source 1..222: mol_type = other DNA
atgttgattttcgtacgcatgttgactggaaagatgatcgagttggaagtgttgccttccgatactatcgagaatgttaaggccaaaatccaagatcatgaagggattcctccagatcaacaacgccttgcttttcaagggaagagcctggaggacggtcgcacactgtctgactataacattcttaaagatcctaaaaagatgccactgctgcgcttgcgt

SEQ ID NO: 721 - DNA, 222 bp, synthetic construct
  source 1..222: mol_type = other DNA
atgttgattttcgtacgcatgttgactggaaagatgatcgagttggaagtgttcccttccgatactatcgagaatgttaaggccaaaatccaagatcatgaagggattcctccagatcaacaacgccttgcttttcaagggaagagcctggaggacggtcgcacactgtctgactataacattcttaaagatcctaaaaagatgccactgctgcgcttgcgt

SEQ ID NO: 722 - DNA, 222 bp, synthetic construct
  source 1..222: mol_type = other DNA
atgttgattttcgtacgcatgttgactggaaagatgatcgagttggaagtggagccttccgatactatcgaggtagttaaggccaaaatccaagatcatgaagggattcctccagatcaacaacgccttgcttttcaagggaagagcctggaggacggtcgcacactgtctgactataacattcttaaagatcctaaaaagatgccactgctgcgcttgcgt

SEQ ID NO: 723 - DNA, 222 bp, synthetic construct
  source 1..222: mol_type = other DNA
atgttgattttcgtacgcatgttgactggaaagatgatcgagttggaagtggagccttccgatactatcgaggaggttaaggccaaaatccaagatcatgaagggattcctccagatcaacaacgccttgcttttcaagggaagagcctggaggacggtcgcacactgtctgactataacattcttaaagatcctaaaaagatgccactgctgcgcttgcgt

SEQ ID NO: 724 - DNA, 222 bp, synthetic construct
  source 1..222: mol_type = other DNA
atgttgattttcgtacgcatgttgactggaaagatgatcgagttggaagtggagccttccgatactatcgagaatattaaggccaaaatccaagatcatgaagggattcctccagatcaacaacgccttgcttttcaagggaagagcctggaggacggtcgcacactgtctgactataacattcttaaagatcctaaaaagatgccactgctgcgcttgcgt

SEQ ID NO: 725 - DNA, 222 bp, synthetic construct
  source 1..222: mol_type = other DNA
atgttgattttcgtacgcatgttgactggaaagatgatcgagttggaagtggagccttccgatactatcgagaatgttaaggccaaaatctgcgatcatgaagggattcctccagatcaacaacgccttgcttttcaagggaagagcctggaggacggtcgcacactgtctgactataacattcttaaagatcctaaaaagatgccactgctgcgcttgcgt

SEQ ID NO: 726 - DNA, 222 bp, synthetic construct
  source 1..222: mol_type = other DNA
atgttgattttcgtacgcatgttgactggaaagatgatcgagttggaagtggagccttccgatactatcgagaatgttaaggccaaaatctgggatcatgaagggattcctccagatcaacaacgccttgcttttcaagggaagagcctggaggacggtcgcacactgtctgactataacattcttaaagatcctaaaaagatgccactgctgcgcttgcgt

SEQ ID NO: 727 - DNA, 222 bp, synthetic construct
  source 1..222: mol_type = other DNA
atgttgattttcgtacgcatgttgactggaaagatgatcgagttggaagtggagccttccgatactatcgagaatgttaaggccaaaatcttcgatcatgaagggattcctccagatcaacaacgccttgcttttcaagggaagagcctggaggacggtcgcacactgtctgactataacattcttaaagatcctaaaaagatgccactgctgcgcttgcgt

SEQ ID NO: 728 - DNA, 222 bp, synthetic construct
  source 1..222: mol_type = other DNA
atgttgattttcgtacgcatgttgactggaaagatgatcgagttggaagtggagccttccgatactatcgagaatgttaaggccaaaatccaagcccatgaagggattcctccagatcaacaacgccttgcttttcaagggaagagcctggaggacggtcgcacactgtctgactataacattcttaaagatcctaaaaagatgccactgctgcgcttgcgt

SEQ ID NO: 729 - DNA, 222 bp, synthetic construct
  source 1..222: mol_type = other DNA
atgttgattttcgtacgcatgttgactggaaagatgatcgagttggaagtggagccttccgatactatcgagaatgttaaggccaaaatccaagattctgaagggattcctccagatcaacaacgccttgcttttcaagggaagagcctggaggacggtcgcacactgtctgactataacattcttaaagatcctaaaaagatgccactgctgcgcttgcgt

SEQ ID NO: 730 - DNA, 222 bp, synthetic construct
  source 1..222: mol_type = other DNA
atgttgattttcgtacgcatgttgactggaaagatgatcgagttggaagtggagccttccgatactatcgagaatgttaaggccaaaatccaagatcaagaagggattcctccagatcaacaacgccttgcttttcaagggaagagcctggaggacggtcgcacactgtctgactataacattcttaaagatcctaaaaagatgccactgctgcgcttgcgt

SEQ ID NO: 731 - DNA, 222 bp, synthetic construct
  source 1..222: mol_type = other DNA
atgttgattttcgtacgcatgttgactggaaagatgatcgagttggaagtggagccttccgatactatcgagaatgttaaggccaaaatccaagatgccgaagggattcctccagatcaacaacgccttgcttttcaagggaagagcctggaggacggtcgcacactgtctgactataacattcttaaagatcctaaaaagatgccactgctgcgcttgcgt

SEQ ID NO: 732 - DNA, 222 bp, synthetic construct
  source 1..222: mol_type = other DNA
atgttgattttcgtacgcatgttgactggaaagatgatcgagttggaagtggagccttccgatactatcgagaatgttaaggccaaaatccaagatcatgaagggattcctttggatcaacaacgccttgcttttcaagggaagagcctggaggacggtcgcacactgtctgactataacattcttaaagatcctaaaaagatgccactgctgcgcttgcgt

SEQ ID NO: 733 - DNA, 222 bp, synthetic construct
  source 1..222: mol_type = other DNA
atgttgattttcgtacgcatgttgactggaaagatgatcgagttggaagtggagccttccgatactatcgagaatgttaaggccaaaatccaagatcatgaagggattccttgcgatcaacaacgccttgcttttcaagggaagagcctggaggacggtcgcacactgtctgactataacattcttaaagatcctaaaaagatgccactgctgcgcttgcgt

SEQ ID NO: 734 - DNA, 222 bp, synthetic construct
  source 1..222: mol_type = other DNA
atgttgattttcgtacgcatgttgactggaaagatgatcgagttggaagtggagccttccgatactatcgagaatgttaaggccaaaatccaagatcatgaagggattcctccagatgagcaacgccttgcttttcaagggaagagcctggaggacggtcgcacactgtctgactataacattcttaaagatcctaaaaagatgccactgctgcgcttgcgt

SEQ ID NO: 735 - DNA, 222 bp, synthetic construct
  source 1..222: mol_type = other DNA
atgttgattttcgtacgcatgttgactggaaagatgatcgagttggaagtggagccttccgatactatcgagaatgttaaggccaaaatccaagatcatgaagggattcctccagatcaacaacatcttgcttttcaagggaagagcctggaggacggtcgcacactgtctgactataacattcttaaagatcctaaaaagatgccactgctgcgcttgcgt

SEQ ID NO: 736 - DNA, 222 bp, synthetic construct
  source 1..222: mol_type = other DNA
atgttgattttcgtacgcatgttgactggaaagatgatcgagttggaagtggagccttccgatactatcgagaatgttaaggccaaaatccaagatcatgaagggattcctccagatcaacaattccttgcttttcaagggaagagcctggaggacggtcgcacactgtctgactataacattcttaaagatcctaaaaagatgccactgctgcgcttgcgt

SEQ ID NO: 737 - DNA, 222 bp, synthetic construct
  source 1..222: mol_type = other DNA
atgttgattttcgtacgcatgttgactggaaagatgatcgagttggaagtggagccttccgatactatcgagaatgttaaggccaaaatccaagatcatgaagggattcctccagatcaacaacgccttacttttcaagggaagagcctggaggacggtcgcacactgtctgactataacattcttaaagatcctaaaaagatgccactgctgcgcttgcgt

SEQ ID NO: 738 - DNA, 222 bp, synthetic construct
  source 1..222: mol_type = other DNA
atgttgattttcgtacgcatgttgactggaaagatgatcgagttggaagtggagccttccgatactatcgagaatgttaaggccaaaatccaagatcatgaagggattcctccagatcaacaacgccttgcttttcaagggactagcctggaggacggtcgcacactgtctgactataacattcttaaagatcctaaaaagatgccactgctgcgcttgcgt

SEQ ID NO: 739 - DNA, 222 bp, synthetic construct
  source 1..222: mol_type = other DNA
atgttgattttcgtacgcatgttgactggaaagatgatcgagttggaagtggagccttccgatactatcgagaatgttaaggccaaaatccaagatcatgaagggattcctccagatcaacaacgccttgcttttcaagggaagttgctggaggacggtcgcacactgtctgactataacattcttaaagatcctaaaaagatgccactgctgcgcttgcgt

SEQ ID NO: 740 - DNA, 222 bp, synthetic construct
  source 1..222: mol_type = other DNA
atgttgattttcgtacgcatgttgactggaaagatgatcgagttggaagtggagccttccgatactatcgagaatgttaaggccaaaatccaagatcatgaagggattcctccagatcaacaacgccttgcttttcaagggaagatgctggaggacggtcgcacactgtctgactataacattcttaaagatcctaaaaagatgccactgctgcgcttgcgt

SEQ ID NO: 741 - DNA, 222 bp, synthetic construct
  source 1..222: mol_type = other DNA
atgttgattttcgtacgcatgttgactggaaagatgatcgagttggaagtggagccttccgatactatcgagaatgttaaggccaaaatccaagatcatgaagggattcctccagatcaacaacgccttgcttttcaagggaagagcctggatgacggtcgcacactgtctgactataacattcttaaagatcctaaaaagatgccactgctgcgcttgcgt

SEQ ID NO: 742 - DNA, 222 bp, synthetic construct
  source 1..222: mol_type = other DNA
atgttgattttcgtacgcatgttgactggaaagatgatcgagttggaagtggagccttccgatactatcgagaatgttaaggccaaaatccaagatcatgaagggattcctccagatcaacaacgccttgcttttcaagggaagagcctggaggacggttatacactgtctgactataacattcttaaagatcctaaaaagatgccactgctgcgcttgcgt

SEQ ID NO: 743 - DNA, 222 bp, synthetic construct
  source 1..222: mol_type = other DNA
atgttgattttcgtacgcatgttgactggaaagatgatcgagttggaagtggagccttccgatactatcgagaatgttaaggccaaaatccaagatcatgaagggattcctccagatcaacaacgccttgcttttcaagggaagagcctggaggacggtcgcacactgggggactataacattcttaaagatcctaaaaagatgccactgctgcgcttgcgt

SEQ ID NO: 744 - DNA, 222 bp, synthetic construct
  source 1..222: mol_type = other DNA
atgttgattttcgtacgcatgttgactggaaagatgatcgagttggaagtggagccttccgatactatcgagaatgttaaggccaaaatccaagatcatgaagggattcctccagatcaacaacgccttgcttttcaagggaagagcctggaggacggtcgcacactgtctgactataacttgcttaaagatcctaaaaagatgccactgctgcgcttgcgt

SEQ ID NO: 745 - DNA, 222 bp, synthetic construct
  source 1..222: mol_type = other DNA
atgttgattttcgtacgcatgttgactggaaagatgatcgagttggaagtggagccttccgatactatcgagaatgttaaggccaaaatccaagatcatgaagggattcctccagatcaacaacgccttgcttttcaagggaagagcctggaggacggtcgcacactgtctgactataacattcttattgatcctaaaaagatgccactgctgcgcttgcgt

SEQ ID NO: 746 - DNA, 222 bp, synthetic construct
  source 1..222: mol_type = other DNA
atgttgattttcgtacgcatgttgactggaaagatgatcgagttggaagtggagccttccgatactatcgagaatgttaaggccaaaatccaagatcatgaagggattcctccagatcaacaacgccttgcttttcaagggaagagcctggaggacggtcgcacactgtctgactataacattcttaaagatcataaaaagatgccactgctgcgcttgcgt

SEQ ID NO: 747 - DNA, 222 bp, synthetic construct
  source 1..222: mol_type = other DNA
atgttgattttcgtacgcatgttgactggaaagatgatcgagttggaagtggagccttccgatactatcgagaatgttaaggccaaaatccaagatcatgaagggattcctccagatcaacaacgccttgcttttcaagggaagagcctggaggacggtcgcacactgtctgactataacattcttaaagatcctaaaaagatgccactgctgcgcatgcgt

SEQ ID NO: 748 - DNA, 222 bp, synthetic construct
  source 1..222: mol_type = other DNA
atgttgattttcgtacgcatgttgactggaaagatgatcgagttggaagtggagccttccgatactatcgagaatgttaaggccaaaatccaagatcatgaagggattcctccagatcaacaacgccttgcttttcaagggaagagcctggaggacggtcgcacactgtctgactataacattcttaaagatcctaaaaagatgccactgctgcgcttgcaa

SEQ ID NO: 749 - DNA, 222 bp, synthetic construct
  source 1..222: mol_type = other DNA
atgttgattttcgtacgcatgttgactggaaagatgatcgagttggaagtggagccttccgatactatcgagaatgttaaggccaaaatccaagatcatgaagggattcctccagatcaacaacgccttgcttttcaagggaagagcctggaggacggtcgcacactgtctgactataacattcttaaagatcctaaaaagatgttggtactgcgcttgcgt

SEQ ID NO: 750 - DNA, 222 bp, synthetic construct
  source 1..222: mol_type = other DNA
atgttgattttcgtacgcatgttgactggaaagatgatcgagttggaagtgtatccttccgatactatcgagaatgttaaggccaaaatccaagatcatgaagggattcctttggatcaacaacgccttgcttttcaagggaagttgctggaggacggtcgcacactgggggactataacattcttaaagatcctaaaaagatgccactgctgcgcttgcgt

SEQ ID NO: 751 - DNA, 222 bp, synthetic construct
  source 1..222: mol_type = other DNA
catttgattttcgtacgcatgttgactggaaagatgatcgagttggaagtggagccttccgatactatcgagaatgttaaggccaaaatccaagatcatgaagggattcctccagatcaacaacgccttgcttttcaagggaagagcctggaggacggtcgcacactgtctgactataacattcttaaagatcctaaaaagatgccactgctgcgcttgcaa

SEQ ID NO: 752 - DNA, 222 bp, synthetic construct
  source 1..222: mol_type = other DNA
tatttgattttcgtacgcatgttgactggaaagatgatcgagttggaagtggagccttccgatactatcgagaatattaaggccaaaatccaagatcatgaagggattcctccagatcaacaacgccttgcttttcaagggaagagcctggaggacggtcgcacactgtctgactataacattcttaaagatcctaaaaagatgccactgctgcgcatgcgt

SEQ ID NO: 753 - DNA, 222 bp, synthetic construct
  source 1..222: mol_type = other DNA
atgttgattttcgtacgcatgttgactggaaagatgatcgagttggaagtggagccttccgatactatcgaggtagttaaggccaaaatccaagatcatgaagggattcctccagatgagcaacgccttgcttttcaagggaagagcctggatgacggtcgcacactgtctgactataacattcttaaagatcctaaaaagatgccactgctgcgcttgcgt

SEQ ID NO: 754 - DNA, 222 bp, synthetic construct
  source 1..222: mol_type = other DNA
atgttgattttcgtacgcatgttgactggaaagatgatcgagttggaagtggagccttccgatactatcgagaatgttaaggccaaaatccaagatcatgaagggattcctccagatcaacaacgccttgcttttcaagggaagagcctggaggacggtcgcacactgtctgactataacttgcttattgatcctaaaaagatgccactgctgcgcttgcgt

SEQ ID NO: 755 - DNA, 222 bp, synthetic construct
  source 1..222: mol_type = other DNA
atgttgattttcgtacgcatgttgactggaaagatgatcgagttggaagtgatgccttccgatactatcgagaatgttaaggccaaaatccaagatcatgaagggattcctccagatcaacaacgccttgcttttcaagggactagcctggatgacggtcgcacactgggggactataacattcttaaagatcctaaaaagatgccactgctgcgcttgcgt

SEQ ID NO: 756 - DNA, 222 bp, synthetic construct
  source 1..222: mol_type = other DNA
atgttgattttcgtacgcatgttgactggaaagatgatcgagttgatggtggagccttccgatactatcgaggtagttaaggccaaaatccaagatcatgaagggattcctccagatgagcaacgccttgcttttcaagggaagttgctggaggacggtcgcacactgtctgactataacattcttaaagatcctaaaaagatgccactgctgcgcttgcgt

SEQ ID NO: 757 - DNA, 222 bp, synthetic construct
  source 1..222: mol_type = other DNA
atgttgattttcgtacgcatgttgactggaaagatgatcgagttggaagtggagccttccgatactatcgagaatattaaggccaaaatccaagatcatgaagggattcctccagatcaatatcgccttgcttttcaagggaagttgctggaggacggtcgcacactgggggactataacattcttaaagatcctaaaaagatgccactgctgcgcttgcgt

SEQ ID NO: 758 - DNA, 222 bp, synthetic construct
  source 1..222: mol_type = other DNA
atgttgattttcgtacgcatgttgactggaaagatgatcgagttggaagtggagccttccgatactatcgagaatgttaaggccaaaatctgcgatcatgaagggattcctccagatcaacaacatcttgcttttcaagggaagagcctggaggacggtcgcacactgggggactataacattcttaaagatcctaaaaagatgccactgctgcgcttgcgt

SEQ ID NO: 759 - DNA, 222 bp, synthetic construct
  source 1..222: mol_type = other DNA
atgttgattttcgtacgcatgttgactggaaagatgatcgagttggaagtgatgccttccgatactatcgaggtagttaaggccaaaatcttcgatcatgaagggattcctccagatcaacaacatcttgcttttcaagggactagcctggaggacggttatacactgggggactataacattcttaaagatcctaaaaagatgccactgctgcgcttgcgt

SEQ ID NO: 760 - DNA, 222 bp, synthetic construct
  source 1..222: mol_type = other DNA
atgttgattttcgtacgcatgttgactggaaagatgatcgagttggaagtgatgccttccgatactatcgaggtagttaaggccaaaatcttcgatcatgaagggattcctccagatcaacaacatcttacttttcaagggactttgctggaggacggttatacactgggggactataacattcttaaagatcctaaaaagatgccactgctgcgcttgcgt

SEQ ID NO: 761 - DNA, 222 bp, synthetic construct
  source 1..222: mol_type = other DNA
atgttgattttcgtacgcatgttgactggaaagatgatcgagttggaagtgatgccttccgatactatcgaggtagttaaggccaaaatcttcgatcatgaagggattcctttggatcaacaacatcttgcttttcaagggactagcctggaggacggttatacactgggggactataacattcttaaagatcctaaaaagatgccactgctgcgcttgcgt

SEQ ID NO: 762 - DNA, 222 bp, synthetic construct
  source 1..222: mol_type = other DNA
atgttgattttcgtacgcatgttgactggaaagatgatcgagttggaagtgatgccttccgatactatcgaggtagttaaggccaaaatccaagatcatgaagggattcctccagatcaacaacatcttgcttttcaagggactagcctggaggacggttatacactgggggactataacattcttaaagatcctaaaaagatgccactgctgcgcttgcgt

SEQ ID NO: 763 - DNA, 222 bp, synthetic construct
  source 1..222: mol_type = other DNA
tatttgattttcgtacgcatgttgactggaaagatgatcgagttggaagtggagccttccgatactatcgagaatattaaggccaaaatccaagatcatgaagggattcctccagatcaacaacgccttgcttttcaagggaagagcctggaggacggtcgcacactgtctgactataacttgcttattgatcctaaaaagatgccactgctgcgcatgcgt

SEQ ID NO: 764 - DNA, 222 bp, synthetic construct
  source 1..222: mol_type = other DNA
tatttgattttcgtacgcatgttgactggaaagatgatcgagttggaagtggagccttccgatactatcgaggtaattaaggccaaaatccaagatcatgaagggattcctccagatgagcaacgccttgcttttcaagggaagagcctggatgacggtcgcacactgtctgactataacttgcttattgatcctaaaaagatgccactgctgcgcatgcgt

SEQ ID NO: 765 - DNA, 222 bp, synthetic construct
  source 1..222: mol_type = other DNA
atgttgattttcgtacgcatgttgactggaaagatgatcgagttggaagtggagccttccgatactatcgaggtagttaaggccaaaatccaagatcatgaagggattcctccagatgagcaacgccttgcttttcaagggaagagcctggatgacggtcgcacactgtctgactataacttgcttattgatcctaaaaagatgccactgctgcgcttgcgt

SEQ ID NO: 766 - DNA, 222 bp, synthetic construct
  source 1..222: mol_type = other DNA
atgttgattttcgtacgcatgttgactggaaagatgatcgagttggaagtgatgccttccgatactatcgagaatgttaaggccaaaatccaagatcatgaagggattcctccagatcaacaacgccttgcttttcaagggactagcctggatgacggtcgcacactgggggactataacttgcttattgatcctaaaaagatgccactgctgcgcttgcgt

SEQ ID NO: 767 - DNA, 222 bp, synthetic construct
  source 1..222: mol_type = other DNA
atgttgattttcgtacgcatgttgactggaaagatgatcgagttgatggtggagccttccgatactatcgaggtagttaaggccaaaatccaagatcatgaagggattcctccagatgagcaacgccttgcttttcaagggaagttgctggaggacggtcgcacactgtctgactataacttgcttattgatcctaaaaagatgccactgctgcgcttgcgt

SEQ ID NO: 768 - DNA, 222 bp, synthetic construct
  source 1..222: mol_type = other DNA
tatttgattttcgtacgcatgttgactggaaagatgatcgagttggaagtgatgccttccgatactatcgagaatattaaggccaaaatccaagatcatgaagggattcctccagatcaacaacgccttgcttttcaagggactagcctggatgacggtcgcacactgggggactataacttgcttattgatcctaaaaagatgccactgctgcgcatgcgt

SEQ ID NO: 769 - DNA, 222 bp, synthetic construct
  source 1..222: mol_type = other DNA
tatttgattttcgtacgcatgttgactggaaagatgatcgagttggaagtgatgccttccgatactatcgagaatattaaggccaaaatccaagatcatgaagggattcctccagatcaacaacgccttgcttttcaagggactagcctggatgacggtcgcacactgggggactataacttgcttattgatcctaaaaagatgccactgctgcgcatgcaa

SEQ ID NO: 770 - DNA, 222 bp, synthetic construct
  source 1..222: mol_type = other DNA
tatttgattttcgtacgcatgttgactggaaagatgatcgagttggaagtgatgccttccgatactatcgaggtaattaaggccaaaatccaagatcatgaagggattcctccagatgagcaacgccttgcttttcaagggactagcctggatgacggtcgcacactgggggactataacttgcttattgatcctaaaaagatgccactgctgcgcatgcaa

SEQ ID NO: 771 - DNA, 222 bp, synthetic construct
  source 1..222: mol_type = other DNA
atgttgattttcgtacgcatgttgactggaaagatgatcgagttggaagtggagccttccgatactatcgagaatgttaaggccaaaatccaagatcatgaagggattcctccagatcaacaacgccttacttttcaagggaagttgctggaggacggtcgcacactgtctgactataacttgcttattgatcctaaaaagatgccactgctgcgcttgcgt

SEQ ID NO: 772 - DNA, 222 bp, synthetic construct
  source 1..222: mol_type = other DNA
atgttgattttcgtacgcatgttgactggaaagatgatcgagttggaagtggagccttccgatactatcgagaatgttaaggccaaaatccaagatcatgaagggattcctttggatcaacaacgccttacttttcaagggaagttgctggaggacggtcgcacactgtctgactataacttgcttattgatcctaaaaagatgccactgctgcgcttgcgt

SEQ ID NO: 773 - DNA, 222 bp, synthetic construct
  source 1..222: mol_type = other DNA
atgttgattttcgtacgcatgttgactggaaagatgatcgagttggaagtggagccttccgatactatcgagaatgttaaggccaaaatccaagatcatgaagggattcctttggatcaacaacgccttacttttcaagggaagttgctggaggacggtcgcacactgtctgactataacttgcttattgatcctaaaaagatgccactgctgcgcttgcaa

SEQ ID NO: 774 - DNA, 222 bp, synthetic construct
  source 1..222: mol_type = other DNA
tatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgccactgctgcgcatgcaa

SEQ ID NO: 775 - DNA, 222 bp, synthetic construct
  source 1..222: mol_type = other DNA
atgttgattttcgtacgcatgttgactggaaagatgatcgagttggaagtggagccttccgatactatcgaggtagttaaggccaaaatccaagatcatgaagggattcctttggatgagcaacgccttacttttcaagggaagttgctggatgacggtcgcacactgtctgactataacttgcttattgatcctaaaaagatgccactgctgcgcttgcaa

SEQ ID NO: 776 - DNA, 222 bp, synthetic construct
  source 1..222: mol_type = other DNA
tatttgattttcgtacgcatgttgactggaaagatgatcgagttggaagtggagccttccgatactatcgaggtaattaaggccaaaatccaagatcatgaagggattcctttggatgagcaacgccttacttttcaagggaagttgctggatgacggtcgcacactgtctgactataacttgcttattgatcctaaaaagatgccactgctgcgcatgcaa

SEQ ID NO: 777 - DNA, 222 bp, synthetic construct
  source 1..222: mol_type = other DNA
atgttgattttcgtacgcatgttgactggaaagatgatcgagttggaagtgatgccttccgatactatcgaggtagttaaggccaaaatccaagatcatgaagggattcctccagatcaacaacatcttacttttcaagggactttgctggaggacggttatacactgggggactataacttgcttattgatcctaaaaagatgccactgctgcgcttgcgt

SEQ ID NO: 778 - DNA, 222 bp, synthetic construct
  source 1..222: mol_type = other DNA
atgttgattttcgtacgcatgttgactggaaagatgatcgagttggaagtgatgccttccgatactatcgaggtagttaaggccaaaatccaagatcatgaagggattcctttggatcaacaacatcttacttttcaagggactttgctggaggacggttatacactgggggactataacttgcttattgatcctaaaaagatgccactgctgcgcttgcgt

SEQ ID NO: 779 - DNA, 222 bp, synthetic construct
  source 1..222: mol_type = other DNA
atgttgattttcgtacgcatgttgactggaaagatgatcgagttggaagtgatgccttccgatactatcgaggtagttaaggccaaaatccaagatcatgaagggattcctttggatcaacaacatcttacttttcaagggactttgctggaggacggttatacactgggggactataacttgcttattgatcctaaaaagatgccactgctgcgcttgcaa

SEQ ID NO: 780 - DNA, 222 bp, synthetic construct
  source 1..222: mol_type = other DNA
atgttgattttcgtacgcatgttgactggaaagatgatcgagttggaagtgatgccttccgatactatcgaggtagttaaggccaaaatccaagatcatgaagggattcctttggatgagcaacatcttacttttcaagggactttgctggatgacggttatacactgggggactataacttgcttattgatcctaaaaagatgccactgctgcgcttgcaa

SEQ ID NO: 781 - DNA, 222 bp, synthetic construct
  source 1..222: mol_type = other DNA
tatttgattttcgtacgcatgttgactggaaagatgatcgagttggaagtgatgccttccgatactatcgaggtaattaaggccaaaatccaagatcatgaagggattcctttggatgagcaacatcttacttttcaagggactttgctggatgacggttatacactgggggactataacttgcttattgatcctaaaaagatgccactgctgcgcatgcaa

SEQ ID NO: 782 - DNA, 222 bp, synthetic construct
  source 1..222: mol_type = other DNA
tatttgattttcgtacgcatgttgactggaaagatgatcgagttggaagtgatgccttccgatactatcgaggtaattaaggccaaaatccaagatcatgaagggattcctccagatgagcaacgccttgcttttcaagggactagcctggatgacggtcgcacactgggggactataacttgcttattgatcctaaaaagatgttggtactgcgcatgcaa

SEQ ID NO: 783 - DNA, 222 bp, synthetic construct
  source 1..222: mol_type = other DNA
tatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgttggtactgcgcatgcaa

SEQ ID NO: 784 - DNA, 222 bp, synthetic construct
  source 1..222: mol_type = other DNA
atgttgattttcgtacgcatgttgactggaaagatgatcgagttggaagtggagccttccgatactatcgaggtagttaaggccaaaatccaagatcatgaagggattcctttggatgagcaacgccttacttttcaagggaagttgctggatgacggtcgcacactgtctgactataacttgcttattgatcctaaaaagatgttggtactgcgcttgcaa

SEQ ID NO: 785 - DNA, 222 bp, synthetic construct
  source 1..222: mol_type = other DNA
atgttgattttcgtacgcatgttgactggaaagatgatcgagttggaagtgatgccttccgatactatcgaggtagttaaggccaaaatccaagatcatgaagggattcctttggatcaacaacatcttacttttcaagggactttgctggaggacggttatacactgggggactataacttgcttattgatcctaaaaagatgttggtactgcgcttgcaa

SEQ ID NO: 786 - DNA, 222 bp, synthetic construct
  source 1..222: mol_type = other DNA
tatttgattttcgtacgcatgttgactggaaagatgatcgagttggaagtgatgccttccgatactatcgaggtaattaaggccaaaatccaagatcatgaagggattcctttggatgagcaacatcttacttttcaagggactttgctggatgacggttatacactgggggactataacttgcttattgatcctaaaaagatgttggtactgcgcatgcaa

SEQ ID NO: 787 - DNA, 222 bp, synthetic construct
  source 1..222: mol_type = other DNA
atgttgattttcgtacgcatgttgactggaaagatgatcgagttggaagtgatgccttccgatactatcgaggtagttaaggccaaaatccaagatcatgaagggattcctccagatcaacaacatcttgcttttcaagggactagcctggaggacggttatacactgggggactataacttgcttattgatcctaaaaagatgttggtactgcgcttgcgt

SEQ ID NO: 788 - DNA, 222 bp, synthetic construct
  source 1..222: mol_type = other DNA
tatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatggccgtactgcgcatgcaa

SEQ ID NO: 789 - DNA, 222 bp, synthetic construct
  source 1..222: mol_type = other DNA
tatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgcgcgtactgcgcatgcaa

SEQ ID NO: 790 - DNA, 222 bp, synthetic construct
  source 1..222: mol_type = other DNA
tatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgaatgtactgcgcatgcaa

SEQ ID NO: 791 - DNA, 222 bp, synthetic construct
  source 1..222: mol_type = other DNA
tatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatggatgtactgcgcatgcaa

SEQ ID NO: 792 - DNA, 222 bp, synthetic construct
  source 1..222: mol_type = other DNA
tatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgtgcgtactgcgcatgcaa

SEQ ID NO: 793 - DNA, 222 bp, synthetic construct
  source 1..222: mol_type = other DNA
tatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatggaggtactgcgcatgcaa

SEQ ID NO: 794 - DNA, 222 bp, synthetic construct
  source 1..222: mol_type = other DNA
tatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgcaagtactgcgcatgcaa

SEQ ID NO: 795 - DNA, 222 bp, synthetic construct
  source 1..222: mol_type = other DNA
tatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgggggtactgcgcatgcaa

SEQ ID NO: 796 - DNA, 222 bp, synthetic construct
  source 1..222: mol_type = other DNA
tatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgcatgtactgcgcatgcaa

SEQ ID NO: 797 - DNA, 222 bp, synthetic construct
  source 1..222: mol_type = other DNA
tatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgattgtactgcgcatgcaa

SEQ ID NO: 798 - DNA, 222 bp, synthetic construct
  source 1..222: mol_type = other DNA
tatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgaaggtactgcgcatgcaa

SEQ ID NO: 799 - DNA, 222 bp, synthetic construct
  source 1..222: mol_type = other DNA
tatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgatggtactgcgcatgcaa

SEQ ID NO: 800 - DNA, 222 bp, synthetic construct
  source 1..222: mol_type = other DNA
tatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgttcgtactgcgcatgcaa

SEQ ID NO: 801 - DNA, 222 bp, synthetic construct
  source 1..222: mol_type = other DNA
tatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgtctgtactgcgcatgcaa

SEQ ID NO: 802 - DNA, 222 bp, synthetic construct
  source 1..222: mol_type = other DNA
tatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgactgtactgcgcatgcaa

SEQ ID NO: 803 - DNA, 222 bp, synthetic construct
  source 1..222: mol_type = other DNA
tatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgtgggtactgcgcatgcaa

SEQ ID NO: 804 - DNA, 222 bp, synthetic construct
  source 1..222: mol_type = other DNA
tatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgtatgtactgcgcatgcaa

SEQ ID NO: 805 - DNA, 222 bp, synthetic construct
  source 1..222: mol_type = other DNA
tatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatggtagtactgcgcatgcaa

SEQ ID NO: 806 - DNA, 222 bp, synthetic construct
  source 1..222: mol_type = other DNA
tatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgttggccctgcgcatgcaa

SEQ ID NO: 807 - DNA, 222 bp, synthetic construct
  source 1..222: mol_type = other DNA
tatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgttgcgcctgcgcatgcaa

SEQ ID NO: 808 - DNA, 222 bp, synthetic construct
  source 1..222: mol_type = other DNA
tatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgttgaatctgcgcatgcaa

SEQ ID NO: 809 - DNA, 222 bp, synthetic construct
  source 1..222: mol_type = other DNA
tatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgttggatctgcgcatgcaa

SEQ ID NO: 810 - DNA, 222 bp, synthetic construct
  source 1..222: mol_type = other DNA
tatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgttgtgcctgcgcatgcaa

SEQ ID NO: 811 - DNA, 222 bp, synthetic construct
  source 1..222: mol_type = other DNA
tatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgttggagctgcgcatgcaa

SEQ ID NO: 812 - DNA, 222 bp, synthetic construct
  source 1..222: mol_type = other DNA
tatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgttgcaactgcgcatgcaa

SEQ ID NO: 813 - DNA, 222 bp, synthetic construct
  source 1..222: mol_type = other DNA
tatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgttggggctgcgcatgcaa

SEQ ID NO: 814 - DNA, 222 bp, synthetic construct
  source 1..222: mol_type = other DNA
tatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgttgcatctgcgcatgcaa

SEQ ID NO: 815 - DNA, 222 bp, synthetic construct
  source 1..222: mol_type = other DNA
tatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgttgattctgcgcatgcaa

SEQ ID NO: 816 - DNA, 222 bp, synthetic construct
  source 1..222: mol_type = other DNA
tatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgttgaagctgcgcatgcaa

SEQ ID NO: 817 - DNA, 222 bp, synthetic construct
  source 1..222: mol_type = other DNA
tatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgttgatgctgcgcatgcaa

SEQ ID NO: 818 - DNA, 222 bp, synthetic construct
  source 1..222: mol_type = other DNA
tatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgttgttcctgcgcatgcaa

SEQ ID NO: 819 - DNA, 222 bp, synthetic construct
  source 1..222: mol_type = other DNA
tatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgttgcctctgcgcatgcaa

SEQ ID NO: 820 - DNA, 222 bp, synthetic construct
  source 1..222: mol_type = other DNA
tatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgttgtctctgcgcatgcaa

SEQ ID NO: 821 - DNA, 222 bp, synthetic construct
  source 1..222: mol_type = other DNA
tatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgttgactctgcgcatgcaa

SEQ ID NO: 822 - DNA, 222 bp, synthetic construct
  source 1..222: mol_type = other DNA
tatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgttgtggctgcgcatgcaa

SEQ ID NO: 823 - DNA, 222 bp, synthetic construct
  source 1..222: mol_type = other DNA
tatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgttgtatctgcgcatgcaa

SEQ ID NO: 824 - DNA, 222 bp, synthetic construct
  source 1..222: mol_type = other DNA
tatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcctatttctcctgagaagatggccgtactgcgcatgcaa

SEQ ID NO: 825 - DNA, 222 bp, synthetic construct
  source 1..222: mol_type = other DNA
tatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcctatttctcctgagaagatgggggtactgcgcatgcaa

SEQ ID NO: 826 - DNA, 222 bp, synthetic construct
  source 1..222: mol_type = other DNA
tatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcctatttctcctgagaagatgttgatgctgcgcatgcaa

SEQ ID NO: 827 - DNA, 222 bp, synthetic construct
  source 1..222: mol_type = other DNA
tatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcctatttctcctgagaagatggccatgctgcgcatgcaa

SEQ ID NO: 828 - DNA, 222 bp, synthetic construct
  source 1..222: mol_type = other DNA
tatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcctatttctcctgagaagatggccttcctgcgcatgcaa

SEQ ID NO: 829 - DNA, 222 bp, synthetic construct
  source 1..222: mol_type = other DNA
tatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcctatttctcctgagaagatggcctgcctgcgcatgcaa

SEQ ID NO: 830 - DNA, 222 bp, synthetic construct
  source 1..222: mol_type = other DNA
tatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcctatttctcctgagaagatggggatgctgcgcatgcaa

SEQ ID NO: 831 - DNA, 222 bp, synthetic construct
  source 1..222: mol_type = other DNA
tatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcctatttctcctgagaagatggggttcctgcgcatgcaa

SEQ ID NO: 832 - DNA, 222 bp, synthetic construct
  source 1..222: mol_type = other DNA
tatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcctatttctcctgagaagatggggtgcctgcgcatgcaa

SEQ ID NO: 833 - DNA, 222 bp, synthetic construct
  source 1..222: mol_type = other DNA
tatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcctatttctcctgagaagatgtgcatgctgcgcatgcaa

SEQ ID NO: 834 - DNA, 222 bp, synthetic construct
  source 1..222: mol_type = other DNA
tatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcctatttctcctgagaagatgatgatgctgcgcatgcaa

SEQ ID NO: 835 - DNA, 222 bp, synthetic construct
  source 1..222: mol_type = other DNA
tatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcctatttctcctgagaagatgttcatgctgcgcatgcaa

SEQ ID NO: 836 - DNA, 222 bp, synthetic construct
  source 1..222: mol_type = other DNA
tatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttggccatttctcctgagaagatggccgtactgcgcatgcaa

SEQ ID NO: 837 - DNA, 222 bp, synthetic construct
  source 1..222: mol_type = other DNA
tatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcgcatttctcctgagaagatggccgtactgcgcatgcaa

SEQ ID NO: 838 - DNA, 222 bp, synthetic construct
  source 1..222: mol_type = other DNA
tatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgaatatttctcctgagaagatggccgtactgcgcatgcaa

SEQ ID NO: 839 - DNA, 222 bp, synthetic construct
  source 1..222: mol_type = other DNA
tatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttggatatttctcctgagaagatggccgtactgcgcatgcaa

SEQ ID NO: 840 - DNA, 222 bp, synthetic construct
  source 1..222: mol_type = other DNA
tatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgtgcatttctcctgagaagatggccgtactgcgcatgcaa

SEQ ID NO: 841 - DNA, 222 bp, synthetic construct
  source 1..222: mol_type = other DNA
tatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttggagatttctcctgagaagatggccgtactgcgcatgcaa

SEQ ID NO: 842 - DNA, 222 bp, synthetic construct
  source 1..222: mol_type = other DNA
tatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcaaatttctcctgagaagatggccgtactgcgcatgcaa

SEQ ID NO: 843 - DNA, 222 bp, synthetic construct
  source 1..222: mol_type = other DNA
tatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttggggatttctcctgagaagatggccgtactgcgcatgcaa

SEQ ID NO: 844 - DNA, 222 bp, synthetic construct
  source 1..222: mol_type = other DNA
tatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcatatttctcctgagaagatggccgtactgcgcatgcaa

SEQ ID NO: 845 - DNA, 222 bp, synthetic construct
  source 1..222: mol_type = other DNA
tatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgattatttctcctgagaagatggccgtactgcgcatgcaa

SEQ ID NO: 846 - DNA, 222 bp, synthetic construct
  source 1..222: mol_type = other DNA
tatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgaagatttctcctgagaagatggccgtactgcgcatgcaa

SEQ ID NO: 847 - DNA, 222 bp, synthetic construct
  source 1..222: mol_type = other DNA
tatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgatgatttctcctgagaagatggccgtactgcgcatgcaa

SEQ ID NO: 848 - DNA, 222 bp, synthetic construct
  source 1..222: mol_type = other DNA
tatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgttcatttctcctgagaagatggccgtactgcgcatgcaa

SEQ ID NO: 849 - DNA, 222 bp, synthetic construct
  source 1..222: mol_type = other DNA
tatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgtctatttctcctgagaagatggccgtactgcgcatgcaa

SEQ ID NO: 850 - DNA, 222 bp, synthetic construct
  source 1..222: mol_type = other DNA
tatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgactatttctcctgagaagatggccgtactgcgcatgcaa

SEQ ID NO: 851 - DNA, 222 bp, synthetic construct
  source 1..222: mol_type = other DNA
tatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgtggatttctcctgagaagatggccgtactgcgcatgcaa

SEQ ID NO: 852 - DNA, 222 bp, synthetic construct
  source 1..222: mol_type = other DNA
tatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgtatatttctcctgagaagatggccgtactgcgcatgcaa

SEQ ID NO: 853 - DNA, 222 bp, synthetic construct
  source 1..222: mol_type = other DNA
tatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttggtaatttctcctgagaagatggccgtactgcgcatgcaa

SEQ ID NO: 854 - DNA, 222 bp, synthetic construct
  source 1..222: mol_type = other DNA
tatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcctatttctcctgagaagatgtgcttcctgcgcatgcaa

SEQ ID NO: 855 - DNA, 222 bp, synthetic construct
  source 1..222: mol_type = other DNA
tatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcctatttctcctgagaagatgatgttcctgcgcatgcaa

SEQ ID NO: 856 - DNA, 222 bp, synthetic construct
  source 1..222: mol_type = other DNA
tatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcctatttctcctgagaagatgttcttcctgcgcatgcaa

SEQ ID NO: 857 - DNA, 222 bp, synthetic construct
  source 1..222: mol_type = other DNA
tatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcctatttctcctgagaagatgtgctgcctgcgcatgcaa

SEQ ID NO: 858 - DNA, 222 bp, synthetic construct
  source 1..222: mol_type = other DNA
tatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcctatttctcctgagaagatgatgtgcctgcgcatgcaa

SEQ ID NO: 859 - DNA, 222 bp, synthetic construct
  source 1..222: mol_type = other DNA
tatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcctatttctcctgagaagatgttctgcctgcgcatgcaa

SEQ ID NO: 860 - DNA, 222 bp, synthetic construct
  source 1..222: mol_type = other DNA
tatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttggccatttctcctgagaagatggccatgctgcgcatgcaa

SEQ ID NO: 861 - DNA, 222 bp, synthetic construct
  source 1..222: mol_type = other DNA
tatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgtgcatttctcctgagaagatggccatgctgcgcatgcaa

SEQ ID NO: 862 - DNA, 222 bp, synthetic construct
  source 1..222: mol_type = other DNA
tatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgactatttctcctgagaagatggccatgctgcgcatgcaa

SEQ ID NO: 863 - DNA, 222 bp, synthetic construct
  source 1..222: mol_type = other DNA
tatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttggtaatttctcctgagaagatggccatgctgcgcatgcaa

SEQ ID NO: 864 - DNA, 222 bp, synthetic construct
  source 1..222: mol_type = other DNA
tatgccattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgactatttctcctgagaagatggccgtactgcgcatgcaa

SEQ ID NO: 865 - DNA, 222 bp, synthetic construct
  source 1..222: mol_type = other DNA
tatcgcattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgactatttctcctgagaagatggccgtactgcgcatgcaa

SEQ ID NO: 866 - DNA, 222 bp, synthetic construct
  source 1..222: mol_type = other DNA
tataatattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgactatttctcctgagaagatggccgtactgcgcatgcaa

SEQ ID NO: 867 - DNA, 222 bp, synthetic construct
  source 1..222: mol_type = other DNA
tatgatattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgactatttctcctgagaagatggccgtactgcgcatgcaa

SEQ ID NO: 868 - DNA, 222 bp, synthetic construct
  source 1..222: mol_type = other DNA
tattgcattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgactatttctcctgagaagatggccgtactgcgcatgcaa

SEQ ID NO: 869 - DNA, 222 bp, synthetic construct
  source 1..222: mol_type = other DNA
tatgagattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgactatttctcctgagaagatggccgtactgcgcatgcaa

SEQ ID NO: 870 - DNA, 222 bp, synthetic construct
  source 1..222: mol_type = other DNA
tatcaaattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgactatttctcctgagaagatggccgtactgcgcatgcaa

SEQ ID NO: 871 - DNA, 222 bp, synthetic construct
  source 1..222: mol_type = other DNA
tatgggattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgactatttctcctgagaagatggccgtactgcgcatgcaa

SEQ ID NO: 872 - DNA, 222 bp, synthetic construct
  source 1..222: mol_type = other DNA
tatcatattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgactatttctcctgagaagatggccgtactgcgcatgcaa

SEQ ID NO: 873 - DNA, 222 bp, synthetic construct
  source 1..222: mol_type = other DNA
tatattattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgactatttctcctgagaagatggccgtactgcgcatgcaa

SEQ ID NO: 874 - DNA, 222 bp, synthetic construct
  source 1..222: mol_type = other DNA
tataagattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgactatttctcctgagaagatggccgtactgcgcatgcaa

SEQ ID NO: 875 - DNA, 222 bp, synthetic construct
  source 1..222: mol_type = other DNA
tatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgactatttctcctgagaagatggccgtactgcgcatgcaa

SEQ ID NO: 876 - DNA, 222 bp, synthetic construct
  source 1..222: mol_type = other DNA
tatttcattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgactatttctcctgagaagatggccgtactgcgcatgcaa

SEQ ID NO: 877 - DNA, 222 bp, synthetic construct
  source 1..222: mol_type = other DNA
tattctattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgactatttctcctgagaagatggccgtactgcgcatgcaa

SEQ ID NO: 878 - DNA, 222 bp, synthetic construct
  source 1..222: mol_type = other DNA
tatactattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgactatttctcctgagaagatggccgtactgcgcatgcaa

SEQ ID NO: 879 - DNA, 222 bp, synthetic construct
  source 1..222: mol_type = other DNA
tattggattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgactatttctcctgagaagatggccgtactgcgcatgcaa

SEQ ID NO: 880 - DNA, 222 bp, synthetic construct
  source 1..222: mol_type = other DNA
tattatattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgactatttctcctgagaagatggccgtactgcgcatgcaa

SEQ ID NO: 881 - DNA, 222 bp, synthetic construct
  source 1..222: mol_type = other DNA
tatgtaattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgactatttctcctgagaagatggccgtactgcgcatgcaa

SEQ ID NO: 882 - DNA, 222 bp, synthetic construct
  source 1..222: mol_type = other DNA
tatcctattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgactatttctcctgagaagatggccgtactgcgcatgcaa

SEQ ID NO: 883 - DNA, 255 bp, synthetic construct
  source 1..255: mol_type = other DNA
atgcaccatcaccaccaccacggtggatctggcatgttgattttcgtaaagacgttgactggaaagactatcactttggaagtggagccttccgatactatcgagaatgttaaggccaaaatccaagataaggaagggattcctccagatcaacaacgccttgcttttgccgggaagagcctggaggacggtcgcacactgtctgactataacattcttaaagattctaaattgcatccactgctgcgcttgcgt

SEQ ID NO: 884 - DNA, 255 bp, synthetic construct
  source 1..255: mol_type = other DNA
atgcaccatcaccaccaccacggggggtcgggcatgttgattttcgtaaagacgttgactggaaagactatcactttggaagtggagccttccgatactatcgagaatgttaaggccaaaatccaagataaggaagggattcctccagatcaacaacgccttgcttttgccgggaagagcctggaggacggtcgcacactgtctgactataacattcttaaagattctaaattgcatctggttctgcgcttgcgt

SEQ ID NO: 885 - DNA, 255 bp, synthetic construct
  source 1..255: mol_type = other DNA
atgcaccatcaccaccaccacggggggtcgggcatgttgattttcgtacgcacgttgactggaaagactatcactttggaagtggagccttccgatactatcgagaatgttaaggccaaaatccaagataaggaagggattcctccagatcaacaacgccttgcttttgccgggaagagcctggaggacggtcgcacactgtctgactataacattcttaaagattctaaattgcatccactgctgcgcttgcgt

SEQ ID NO: 886 - DNA, 255 bp, synthetic construct
  source 1..255: mol_type = other DNA
atgcaccatcaccaccaccacggggggtcgggcatgttgattttcgtaaagacgttgactggaaagactatcgagttggaagtggagccttccgatactatcgagaatgttaaggccaaaatccaagataaggaagggattcctccagatcaacaacgccttgcttttgccgggaagagcctggaggacggtcgcacactgtctgactataacattcttaaagattctaaattgcatccactgctgcgcttgcgt

SEQ ID NO: 887 - DNA, 255 bp, synthetic construct
  source 1..255: mol_type = other DNA
atgcaccatcaccaccaccacggggggtcgggcatgttgattttcgtaaagacgttgactggaaagactatcactttggaagtggagccttccgatactatcgagaatgttaaggccaaaatccaagatgccgaagggattcctccagatcaacaacgccttgcttttgccgggaagagcctggaggacggtcgcacactgtctgactataacattcttaaagattctaaattgcatccactgctgcgcttgcgt

SEQ ID NO: 888 - DNA, 255 bp, synthetic construct
  source 1..255: mol_type = other DNA
atgcaccatcaccaccaccacggggggtcgggcatgttgattttcgtaaagacgttgactggaaagactatcactttggaagtggagccttccgatactatcgagaatgttaaggccaaaatccaagataaggaagggattcctccagatcaacaacgccttgcttttcaagggaagagcctggaggacggtcgcacactgtctgactataacattcttaaagattctaaattgcatccactgctgcgcttgcgt

SEQ ID NO: 889 - DNA, 255 bp, synthetic construct
  source 1..255: mol_type = other DNA
atgcaccatcaccaccaccacggggggtcgggcatgttgattttcgtaaagacgttgactggaaagactatcactttggaagtggagccttccgatactatcgagaatgttaaggccaaaatccaagataaggaagggattcctccagatcaacaacgccttgcttttgccgggaagagcctggaggacggtcgcacactgtctgactataacattcttattgattctaaattgcatccactgctgcgcttgcgt

SEQ ID NO: 890 - DNA, 255 bp, synthetic construct
  source 1..255: mol_type = other DNA
atgcaccatcaccaccaccacggggggtcgggcatgttgattttcgtaaagacgttgactggaaagactatcactttggaagtggagccttccgatactatcgagaatgttaaggccaaaatccaagataaggaagggattcctccagatcaacaacgccttgcttttgccgggaagagcctggaggacggtcgcacactgtctgactataacattcttaaagatcctaaattgcatccactgctgcgcttgcgt

SEQ ID NO: 891 - DNA, 1542 bp, synthetic construct
  source 1..1542: mol_type = other DNA
atgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgaggaaaatctgtattttcagggccacatgaatagctttgttggtctgcgtgttgttgcaaaatggtcaagcaatggttatttctacagcggcaaaatcacccgtgatgttggtgcaggtaaatacaaactgctgtttgatgatggttatgaatgtgatgtgctgggcaaagatattctgctgtgtgatccgattccgctggataccgaagttaccgcactgagcgaagatgaatatttcagtgccggtgttgttaaaggccatcgtaaagaaagcggtgaactgtattacagcattgaaaaagaaggtcagcgcaaatggtataaacgtatggcagttattctgagcctggaacagggtaatcgtctgcgtgaacagtatggtctgggt

SEQ ID NO: 892 - DNA, 255 bp, synthetic construct
  source 1..255: mol_type = other DNA
atgcaccaccaccaccaccacggtggatctggccatttgattttcgtaaagacgttgactggaaagactatcactttggaagtggagccttccgatactatcgagaatgttaaggccaaaatccaagataaggaagggattcctccagatcaacaacgccttgcttttgccgggaagagcctggaggacggtcgcacactgtctgactataacattcttaaagattctaaattgcatccactgctgcgcttgcgt

SEQ ID NO: 893 - DNA, 255 bp, synthetic construct
  source 1..255: mol_type = other DNA
atgcaccaccaccaccaccacggtggatctggcatgttgattttcgtacgcacgttgactggaaagactatcactttggaagtggagccttccgatactatcgagaatgttaaggccaaaatccaagataaggaagggattcctccagatcaacaacgccttgcttttgccgggaagagcctggaggacggtcgcacactgtctgactataacattcttaaagattctaaattgcatccactgctgcgcttgcgt

SEQ ID NO: 894 - DNA, 255 bp, synthetic construct
  source 1..255: mol_type = other DNA
atgcaccaccaccaccaccacggtggatctggcatgttgattttcgtaaagatgttgactggaaagactatcactttggaagtggagccttccgatactatcgagaatgttaaggccaaaatccaagataaggaagggattcctccagatcaacaacgccttgcttttgccgggaagagcctggaggacggtcgcacactgtctgactataacattcttaaagattctaaattgcatccactgctgcgcttgcgt

SEQ ID NO: 895 - DNA, 255 bp, synthetic construct
  source 1..255: mol_type = other DNA
atgcaccaccaccaccaccacggtggatctggcatgttgattttcgtaaagacgttgactggaaagatgatcactttggaagtggagccttccgatactatcgagaatgttaaggccaaaatccaagataaggaagggattcctccagatcaacaacgccttgcttttgccgggaagagcctggaggacggtcgcacactgtctgactataacattcttaaagattctaaattgcatccactgctgcgcttgcgt

SEQ ID NO: 896 - DNA, 255 bp, synthetic construct
  source 1..255: mol_type = other DNA
atgcaccaccaccaccaccacggtggatctggcatgttgattttcgtaaagacgttgactggaaagactatcgagttggaagtggagccttccgatactatcgagaatgttaaggccaaaatccaagataaggaagggattcctccagatcaacaacgccttgcttttgccgggaagagcctggaggacggtcgcacactgtctgactataacattcttaaagattctaaattgcatccactgctgcgcttgcgt

SEQ ID NO: 897 - DNA, 255 bp, synthetic construct
  source 1..255: mol_type = other DNA
atgcaccaccaccaccaccacggtggatctggcatgttgattttcgtaaagacgttgactggaaagactatcactttgatggtggagccttccgatactatcgagaatgttaaggccaaaatccaagataaggaagggattcctccagatcaacaacgccttgcttttgccgggaagagcctggaggacggtcgcacactgtctgactataacattcttaaagattctaaattgcatccactgctgcgcttgcgt

SEQ ID NO: 898 - DNA, 255 bp, synthetic construct
  source 1..255: mol_type = other DNA
atgcaccaccaccaccaccacggtggatctggcatgttgattttcgtaaagacgttgactggaaagactatcactttggaagtgatgccttccgatactatcgagaatgttaaggccaaaatccaagataaggaagggattcctccagatcaacaacgccttgcttttgccgggaagagcctggaggacggtcgcacactgtctgactataacattcttaaagattctaaattgcatccactgctgcgcttgcgt

SEQ ID NO: 899 - DNA, 255 bp, synthetic construct
  source 1..255: mol_type = other DNA
atgcaccaccaccaccaccacggtggatctggcatgttgattttcgtaaagacgttgactggaaagactatcactttggaagtggagccttccgatactatcgaggtagttaaggccaaaatccaagataaggaagggattcctccagatcaacaacgccttgcttttgccgggaagagcctggaggacggtcgcacactgtctgactataacattcttaaagattctaaattgcatccactgctgcgcttgcgt

SEQ ID NO: 900 - DNA, 255 bp, synthetic construct
  source 1..255: mol_type = other DNA
atgcaccaccaccaccaccacggtggatctggcatgttgattttcgtaaagacgttgactggaaagactatcactttggaagtggagccttccgatactatcgagaatattaaggccaaaatccaagataaggaagggattcctccagatcaacaacgccttgcttttgccgggaagagcctggaggacggtcgcacactgtctgactataacattcttaaagattctaaattgcatccactgctgcgcttgcgt

SEQ ID NO: 901 - DNA, 255 bp, synthetic construct
  source 1..255: mol_type = other DNA
atgcaccaccaccaccaccacggtggatctggcatgttgattttcgtaaagacgttgactggaaagactatcactttggaagtggagccttccgatactatcgagaatgttaaggccaaaatctgggataaggaagggattcctccagatcaacaacgccttgcttttgccgggaagagcctggaggacggtcgcacactgtctgactataacattcttaaagattctaaattgcatccactgctgcgcttgcgt

SEQ ID NO: 902 - DNA, 255 bp, synthetic construct
  source 1..255: mol_type = other DNA
atgcaccaccaccaccaccacggtggatctggcatgttgattttcgtaaagacgttgactggaaagactatcactttggaagtggagccttccgatactatcgagaatgttaaggccaaaatctgcgataaggaagggattcctccagatcaacaacgccttgcttttgccgggaagagcctggaggacggtcgcacactgtctgactataacattcttaaagattctaaattgcatccactgctgcgcttgcgt

SEQ ID NO: 903 - DNA, 255 bp, synthetic construct
  source 1..255: mol_type = other DNA
atgcaccaccaccaccaccacggtggatctggcatgttgattttcgtaaagacgttgactggaaagactatcactttggaagtggagccttccgatactatcgagaatgttaaggccaaaatcttcgataaggaagggattcctccagatcaacaacgccttgcttttgccgggaagagcctggaggacggtcgcacactgtctgactataacattcttaaagattctaaattgcatccactgctgcgcttgcgt

SEQ ID NO: 904 - DNA, 255 bp, synthetic construct
  source 1..255: mol_type = other DNA
atgcaccaccaccaccaccacggtggatctggcatgttgattttcgtaaagacgttgactggaaagactatcactttggaagtggagccttccgatactatcgagaatgttaaggccaaaatccaagattctgaagggattcctccagatcaacaacgccttgcttttgccgggaagagcctggaggacggtcgcacactgtctgactataacattcttaaagattctaaattgcatccactgctgcgcttgcgt

SEQ ID NO: 905 - DNA, 255 bp, synthetic construct
  source 1..255: mol_type = other DNA
atgcaccaccaccaccaccacggtggatctggcatgttgattttcgtaaagacgttgactggaaagactatcactttggaagtggagccttccgatactatcgagaatgttaaggccaaaatccaagatcatgaagggattcctccagatcaacaacgccttgcttttgccgggaagagcctggaggacggtcgcacactgtctgactataacattcttaaagattctaaattgcatccactgctgcgcttgcgt

SEQ ID NO: 906 - DNA, 255 bp, synthetic construct
  source 1..255: mol_type = other DNA
atgcaccaccaccaccaccacggtggatctggcatgttgattttcgtaaagacgttgactggaaagactatcactttggaagtggagccttccgatactatcgagaatgttaaggccaaaatccaagatgccgaagggattcctccagatcaacaacgccttgcttttgccgggaagagcctggaggacggtcgcacactgtctgactataacattcttaaagattctaaattgcatccactgctgcgcttgcgt

SEQ ID NO: 907 - DNA, 255 bp, synthetic construct
  source 1..255: mol_type = other DNA
atgcaccaccaccaccaccacggtggatctggcatgttgattttcgtaaagacgttgactggaaagactatcactttggaagtggagccttccgatactatcgagaatgttaaggccaaaatccaagataaggaagggattcctttggatcaacaacgccttgcttttgccgggaagagcctggaggacggtcgcacactgtctgactataacattcttaaagattctaaattgcatccactgctgcgcttgcgt

SEQ ID NO: 908 - DNA, 255 bp, synthetic construct
  source 1..255: mol_type = other DNA
atgcaccaccaccaccaccacggtggatctggcatgttgattttcgtaaagacgttgactggaaagactatcactttggaagtggagccttccgatactatcgagaatgttaaggccaaaatccaagataaggaagggattcctccagatcaacaatggcttgcttttgccgggaagagcctggaggacggtcgcacactgtctgactataacattcttaaagattctaaattgcatccactgctgcgcttgcgt

SEQ ID NO: 909 - DNA, 255 bp, synthetic construct
  source 1..255: mol_type = other DNA
atgcaccaccaccaccaccacggtggatctggcatgttgattttcgtaaagacgttgactggaaagactatcactttggaagtggagccttccgatactatcgagaatgttaaggccaaaatccaagataaggaagggattcctccagatcaacaacgccttacttttgccgggaagagcctggaggacggtcgcacactgtctgactataacattcttaaagattctaaattgcatccactgctgcgcttgcgt

SEQ ID NO: 910 - DNA, 255 bp, synthetic construct
  source 1..255: mol_type = other DNA
atgcaccaccaccaccaccacggtggatctggcatgttgattttcgtaaagacgttgactggaaagactatcactttggaagtggagccttccgatactatcgagaatgttaaggccaaaatccaagataaggaagggattcctccagatcaacaacgccttgcttttcaagggaagagcctggaggacggtcgcacactgtctgactataacattcttaaagattctaaattgcatccactgctgcgcttgcgt

SEQ ID NO: 911 - DNA, 255 bp, synthetic construct
  source 1..255: mol_type = other DNA
atgcaccaccaccaccaccacggtggatctggcatgttgattttcgtaaagacgttgactggaaagactatcactttggaagtggagccttccgatactatcgagaatgttaaggccaaaatccaagataaggaagggattcctccagatcaacaacgccttgcttttgccgggactagcctggaggacggtcgcacactgtctgactataacattcttaaagattctaaattgcatccactgctgcgcttgcgt

SEQ ID NO: 912 - DNA, 255 bp, synthetic construct
  source 1..255: mol_type = other DNA
atgcaccaccaccaccaccacggtggatctggcatgttgattttcgtaaagacgttgactggaaagactatcactttggaagtggagccttccgatactatcgagaatgttaaggccaaaatccaagataaggaagggattcctccagatcaacaacgccttgcttttgccgggaagagcctggaggacggttatacactgtctgactataacattcttaaagattctaaattgcatccactgctgcgcttgcgt

SEQ ID NO: 913 - DNA, 255 bp, synthetic construct
  source 1..255: mol_type = other DNA
atgcaccaccaccaccaccacggtggatctggcatgttgattttcgtaaagacgttgactggaaagactatcactttggaagtggagccttccgatactatcgagaatgttaaggccaaaatccaagataaggaagggattcctccagatcaacaacgccttgcttttgccgggaagagcctggaggacggtcgcacactgggggactataacattcttaaagattctaaattgcatccactgctgcgcttgcgt

SEQ ID NO: 914 - DNA, 255 bp, synthetic construct
  source 1..255: mol_type = other DNA
atgcaccaccaccaccaccacggtggatctggcatgttgattttcgtaaagacgttgactggaaagactatcactttggaagtggagccttccgatactatcgagaatgttaaggccaaaatccaagataaggaagggattcctccagatcaacaacgccttgcttttgccgggaagagcctggaggacggtcgcacactgtctgactataacattcttattgattctaaattgcatccactgctgcgcttgcgt

SEQ ID NO: 915 - DNA, 255 bp, synthetic construct
  source 1..255: mol_type = other DNA
atgcaccaccaccaccaccacggtggatctggcatgttgattttcgtaaagacgttgactggaaagactatcactttggaagtggagccttccgatactatcgagaatgttaaggccaaaatccaagataaggaagggattcctccagatcaacaacgccttgcttttgccgggaagagcctggaggacggtcgcacactgtctgactataacattcttaaagatcctaaattgcatccactgctgcgcttgcgt

SEQ ID NO: 916 - DNA, 255 bp, synthetic construct
  source 1..255: mol_type = other DNA
atgcaccaccaccaccaccacggtggatctggcatgttgattttcgtacgcatgttgactggaaagatgatcgagttggaagtggagccttccgatactatcgagaatgttaaggccaaaatccaagatcatgaagggattcctccagatcaacaacgccttgcttttcaagggaagagcctggaggacggtcgcacactgtctgactataacattcttaaagatcctaaaaagatgccactgctgcgcttgcgt

SEQ ID NO: 917 - DNA, 255 bp, synthetic construct
  source 1..255: mol_type = other DNA
atgcaccaccaccaccaccacggtggatctggcatgttgattttcgtacgcacgttgactggaaagactatcactttggaagtggagccttccgatactatcgagaatgttaaggccaaaatccaagatcatgaagggattcctccagatcaacaacgccttgcttttcaagggaagagcctggaggacggtcgcacactgtctgactataacattcttaaagatcctaaattgcatccactgctgcgcttgcgt

SEQ ID NO: 918 - DNA, 255 bp, synthetic construct
  source 1..255: mol_type = other DNA
atgcaccatcaccaccaccacggtggatctggcatgttgattttcgtaaagatgttgactggaaagactatcgagttggaagtggagccttccgatactatcgagaatgttaaggccaaaatccaagataaggaagggattcctccagatcaacaacgccttgcttttcaagggaagagcctggaggacggtcgcacactgtctgactataacattcttaaagattctaaaaagcatccactgctgcgcttgcgt

SEQ ID NO: 919 - DNA, 255 bp, synthetic construct
  source 1..255: mol_type = other DNA
atgcaccatcaccaccaccacggtggatctggcatgttgattttcgtaaagacgttgactggaaagatgatcactttggaagtggagccttccgatactatcgagaatgttaaggccaaaatccaagatcatgaagggattcctccagatcaacaacgccttgcttttcaagggaagagcctggaggacggtcgcacactgtctgactataacattcttaaagattctaaattgatgccactgctgcgcttgcgt

SEQ ID NO: 920 - DNA, 255 bp, synthetic construct
  source 1..255: mol_type = other DNA
atgcaccaccaccaccaccacggtggatctggcatgttgattttcgtaaagatgttgactggaaagatgatcgagttggaagtggagccttccgatactatcgagaatgttaaggccaaaatccaagatcatgaagggattcctccagatcaacaacgccttgcttttcaagggaagagcctggaggacggtcgcacactgtctgactataacattcttaaagatcctaaaaagatgccactgctgcgcttgcgt

SEQ ID NO: 921 - DNA, 255 bp, synthetic construct
  source 1..255: mol_type = other DNA
atgcaccaccaccaccaccacggtggatctggcatgttgattttcgtacgcacgttgactggaaagatgatcgagttggaagtggagccttccgatactatcgagaatgttaaggccaaaatccaagatcatgaagggattcctccagatcaacaacgccttgcttttcaagggaagagcctggaggacggtcgcacactgtctgactataacattcttaaagatcctaaaaagatgccactgctgcgcttgcgt

SEQ ID NO: 922 - DNA, 255 bp, synthetic construct
  source 1..255: mol_type = other DNA
atgcaccaccaccaccaccacggtggatctggcatgttgattttcgtacgcatgttgactggaaagactatcgagttggaagtggagccttccgatactatcgagaatgttaaggccaaaatccaagatcatgaagggattcctccagatcaacaacgccttgcttttcaagggaagagcctggaggacggtcgcacactgtctgactataacattcttaaagatcctaaaaagatgccactgctgcgcttgcgt

SEQ ID NO: 923 - DNA, 255 bp, synthetic construct
  source 1..255: mol_type = other DNA
atgcaccaccaccaccaccacggtggatctggcatgttgattttcgtacgcatgttgactggaaagatgatcactttggaagtggagccttccgatactatcgagaatgttaaggccaaaatccaagatcatgaagggattcctccagatcaacaacgccttgcttttcaagggaagagcctggaggacggtcgcacactgtctgactataacattcttaaagatcctaaaaagatgccactgctgcgcttgcgt

SEQ ID NO: 924 - DNA, 255 bp, synthetic construct
  source 1..255: mol_type = other DNA
atgcaccaccaccaccaccacggtggatctggcatgttgattttcgtacgcatgttgactggaaagatgatcgagttggaagtggagccttccgatactatcgagaatgttaaggccaaaatccaagataaggaagggattcctccagatcaacaacgccttgcttttcaagggaagagcctggaggacggtcgcacactgtctgactataacattcttaaagatcctaaaaagatgccactgctgcgcttgcgt

SEQ ID NO: 925 - DNA, 255 bp, synthetic construct
  source 1..255: mol_type = other DNA
atgcaccaccaccaccaccacggtggatctggcatgttgattttcgtacgcatgttgactggaaagatgatcgagttggaagtggagccttccgatactatcgagaatgttaaggccaaaatccaagatcatgaagggattcctccagatcaacaacgccttgcttttgccgggaagagcctggaggacggtcgcacactgtctgactataacattcttaaagatcctaaaaagatgccactgctgcgcttgcgt

SEQ ID NO: 926 - DNA, 255 bp, synthetic construct
  source 1..255: mol_type = other DNA
atgcaccaccaccaccaccacggtggatctggcatgttgattttcgtacgcatgttgactggaaagatgatcgagttggaagtggagccttccgatactatcgagaatgttaaggccaaaatccaagatcatgaagggattcctccagatcaacaacgccttgcttttcaagggaagagcctggaggacggtcgcacactgtctgactataacattcttaaagattctaaaaagatgccactgctgcgcttgcgt

SEQ ID NO: 927 - DNA, 255 bp, synthetic construct
  source 1..255: mol_type = other DNA
atgcaccaccaccaccaccacggtggatctggcatgttgattttcgtacgcatgttgactggaaagatgatcgagttggaagtggagccttccgatactatcgagaatgttaaggccaaaatccaagatcatgaagggattcctccagatcaacaacgccttgcttttcaagggaagagcctggaggacggtcgcacactgtctgactataacattcttaaagatcctaaattgatgccactgctgcgcttgcgt

SEQ ID NO: 928 - DNA, 255 bp, synthetic construct
  source 1..255: mol_type = other DNA
atgcaccaccaccaccaccacggtggatctggcatgttgattttcgtacgcatgttgactggaaagatgatcgagttggaagtggagccttccgatactatcgagaatgttaaggccaaaatccaagatcatgaagggattcctccagatcaacaacgccttgcttttcaagggaagagcctggaggacggtcgcacactgtctgactataacattcttaaagatcctaaaaagcatccactgctgcgcttgcgt

SEQ ID NO: 929 - DNA, 255 bp, synthetic construct
  source 1..255: mol_type = other DNA
atgcaccaccaccaccaccacggtggatctggccatttgattttcgtacgcatgttgactggaaagatgatcgagttggaagtggagccttccgatactatcgagaatgttaaggccaaaatccaagatcatgaagggattcctccagatcaacaacgccttgcttttcaagggaagagcctggaggacggtcgcacactgtctgactataacattcttaaagatcctaaaaagatgccactgctgcgcttgcgt

SEQ ID NO: 930 - DNA, 255 bp, synthetic construct
  source 1..255: mol_type = other DNA
atgcaccaccaccaccaccacggtggatctggctatttgattttcgtacgcatgttgactggaaagatgatcgagttggaagtggagccttccgatactatcgagaatgttaaggccaaaatccaagatcatgaagggattcctccagatcaacaacgccttgcttttcaagggaagagcctggaggacggtcgcacactgtctgactataacattcttaaagatcctaaaaagatgccactgctgcgcttgcgt

SEQ ID NO: 931 - DNA, 255 bp, synthetic construct
  source 1..255: mol_type = other DNA
atgcaccaccaccaccaccacggtggatctggcatgttgattttcgtacgcatgttgactggaaagatgatccatttggaagtggagccttccgatactatcgagaatgttaaggccaaaatccaagatcatgaagggattcctccagatcaacaacgccttgcttttcaagggaagagcctggaggacggtcgcacactgtctgactataacattcttaaagatcctaaaaagatgccactgctgcgcttgcgt

SEQ ID NO: 932 - DNA, 255 bp, synthetic construct
  source 1..255: mol_type = other DNA
atgcaccaccaccaccaccacggtggatctggcatgttgattttcgtacgcatgttgactggaaagatgatcgatttggaagtggagccttccgatactatcgagaatgttaaggccaaaatccaagatcatgaagggattcctccagatcaacaacgccttgcttttcaagggaagagcctggaggacggtcgcacactgtctgactataacattcttaaagatcctaaaaagatgccactgctgcgcttgcgt

SEQ ID NO: 933 - DNA, 255 bp, synthetic construct
  source 1..255: mol_type = other DNA
atgcaccaccaccaccaccacggtggatctggcatgttgattttcgtacgcatgttgactggaaagatgatcgagttgatggtggagccttccgatactatcgagaatgttaaggccaaaatccaagatcatgaagggattcctccagatcaacaacgccttgcttttcaagggaagagcctggaggacggtcgcacactgtctgactataacattcttaaagatcctaaaaagatgccactgctgcgcttgcgt

SEQ ID NO: 934 - DNA, 255 bp, synthetic construct
  source 1..255: mol_type = other DNA
atgcaccaccaccaccaccacggtggatctggcatgttgattttcgtacgcatgttgactggaaagatgatcgagttgactgtggagccttccgatactatcgagaatgttaaggccaaaatccaagatcatgaagggattcctccagatcaacaacgccttgcttttcaagggaagagcctggaggacggtcgcacactgtctgactataacattcttaaagatcctaaaaagatgccactgctgcgcttgcgt

SEQ ID NO: 935 - DNA, 255 bp, synthetic construct
  source 1..255: mol_type = other DNA
atgcaccaccaccaccaccacggtggatctggcatgttgattttcgtacgcatgttgactggaaagatgatcgagttggaagtgatgccttccgatactatcgagaatgttaaggccaaaatccaagatcatgaagggattcctccagatcaacaacgccttgcttttcaagggaagagcctggaggacggtcgcacactgtctgactataacattcttaaagatcctaaaaagatgccactgctgcgcttgcgt

SEQ ID NO: 936 - DNA, 255 bp, synthetic construct
  source 1..255: mol_type = other DNA
atgcaccaccaccaccaccacggtggatctggcatgttgattttcgtacgcatgttgactggaaagatgatcgagttggaagtgtatccttccgatactatcgagaatgttaaggccaaaatccaagatcatgaagggattcctccagatcaacaacgccttgcttttcaagggaagagcctggaggacggtcgcacactgtctgactataacattcttaaagatcctaaaaagatgccactgctgcgcttgcgt

SEQ ID NO: 937 - DNA, 255 bp, synthetic construct
  source 1..255: mol_type = other DNA
atgcaccaccaccaccaccacggtggatctggcatgttgattttcgtacgcatgttgactggaaagatgatcgagttggaagtgttgccttccgatactatcgagaatgttaaggccaaaatccaagatcatgaagggattcctccagatcaacaacgccttgcttttcaagggaagagcctggaggacggtcgcacactgtctgactataacattcttaaagatcctaaaaagatgccactgctgcgcttgcgt

SEQ ID NO: 938 - DNA, 255 bp, synthetic construct
  source 1..255: mol_type = other DNA
atgcaccaccaccaccaccacggtggatctggcatgttgattttcgtacgcatgttgactggaaagatgatcgagttggaagtgttcccttccgatactatcgagaatgttaaggccaaaatccaagatcatgaagggattcctccagatcaacaacgccttgcttttcaagggaagagcctggaggacggtcgcacactgtctgactataacattcttaaagatcctaaaaagatgccactgctgcgcttgcgt

SEQ ID NO: 939 - DNA, 255 bp, synthetic construct
  source 1..255: mol_type = other DNA
atgcaccaccaccaccaccacggtggatctggcatgttgattttcgtacgcatgttgactggaaagatgatcgagttggaagtggagccttccgatactatcgaggtagttaaggccaaaatccaagatcatgaagggattcctccagatcaacaacgccttgcttttcaagggaagagcctggaggacggtcgcacactgtctgactataacattcttaaagatcctaaaaagatgccactgctgcgcttgcgt

SEQ ID NO: 940 - DNA, 255 bp, synthetic construct
  source 1..255: mol_type = other DNA
atgcaccaccaccaccaccacggtggatctggcatgttgattttcgtacgcatgttgactggaaagatgatcgagttggaagtggagccttccgatactatcgaggaggttaaggccaaaatccaagatcatgaagggattcctccagatcaacaacgccttgcttttcaagggaagagcctggaggacggtcgcacactgtctgactataacattcttaaagatcctaaaaagatgccactgctgcgcttgcgt

SEQ ID NO: 941 - DNA, 255 bp, synthetic construct
  source 1..255: mol_type = other DNA
atgcaccaccaccaccaccacggtggatctggcatgttgattttcgtacgcatgttgactggaaagatgatcgagttggaagtggagccttccgatactatcgagaatattaaggccaaaatccaagatcatgaagggattcctccagatcaacaacgccttgcttttcaagggaagagcctggaggacggtcgcacactgtctgactataacattcttaaagatcctaaaaagatgccactgctgcgcttgcgt

SEQ ID NO: 942 - DNA, 255 bp, synthetic construct
  source 1..255: mol_type = other DNA
atgcaccaccaccaccaccacggtggatctggcatgttgattttcgtacgcatgttgactggaaagatgatcgagttggaagtggagccttccgatactatcgagaatgttaaggccaaaatctgcgatcatgaagggattcctccagatcaacaacgccttgcttttcaagggaagagcctggaggacggtcgcacactgtctgactataacattcttaaagatcctaaaaagatgccactgctgcgcttgcgt

SEQ ID NO: 943 - DNA, 255 bp, synthetic construct
  source 1..255: mol_type = other DNA
atgcaccaccaccaccaccacggtggatctggcatgttgattttcgtacgcatgttgactggaaagatgatcgagttggaagtggagccttccgatactatcgagaatgttaaggccaaaatctgggatcatgaagggattcctccagatcaacaacgccttgcttttcaagggaagagcctggaggacggtcgcacactgtctgactataacattcttaaagatcctaaaaagatgccactgctgcgcttgcgt

SEQ ID NO: 944 - DNA, 255 bp, synthetic construct
  source 1..255: mol_type = other DNA
atgcaccaccaccaccaccacggtggatctggcatgttgattttcgtacgcatgttgactggaaagatgatcgagttggaagtggagccttccgatactatcgagaatgttaaggccaaaatcttcgatcatgaagggattcctccagatcaacaacgccttgcttttcaagggaagagcctggaggacggtcgcacactgtctgactataacattcttaaagatcctaaaaagatgccactgctgcgcttgcgt

SEQ ID NO: 945 - DNA, 255 bp, synthetic construct
  source 1..255: mol_type = other DNA
atgcaccaccaccaccaccacggtggatctggcatgttgattttcgtacgcatgttgactggaaagatgatcgagttggaagtggagccttccgatactatcgagaatgttaaggccaaaatccaagcccatgaagggattcctccagatcaacaacgccttgcttttcaagggaagagcctggaggacggtcgcacactgtctgactataacattcttaaagatcctaaaaagatgccactgctgcgcttgcgt

SEQ ID NO: 946 - DNA, 255 bp, synthetic construct
  source 1..255: mol_type = other DNA
atgcaccaccaccaccaccacggtggatctggcatgttgattttcgtacgcatgttgactggaaagatgatcgagttggaagtggagccttccgatactatcgagaatgttaaggccaaaatccaagattctgaagggattcctccagatcaacaacgccttgcttttcaagggaagagcctggaggacggtcgcacactgtctgactataacattcttaaagatcctaaaaagatgccactgctgcgcttgcgt

SEQ ID NO: 947 - DNA, 255 bp, synthetic construct
  source 1..255: mol_type = other DNA
atgcaccaccaccaccaccacggtggatctggcatgttgattttcgtacgcatgttgactggaaagatgatcgagttggaagtggagccttccgatactatcgagaatgttaaggccaaaatccaagatcaagaagggattcctccagatcaacaacgccttgcttttcaagggaagagcctggaggacggtcgcacactgtctgactataacattcttaaagatcctaaaaagatgccactgctgcgcttgcgt

SEQ ID NO: 948 - DNA, 255 bp, synthetic construct
  source 1..255: mol_type = other DNA
atgcaccaccaccaccaccacggtggatctggcatgttgattttcgtacgcatgttgactggaaagatgatcgagttggaagtggagccttccgatactatcgagaatgttaaggccaaaatccaagatgccgaagggattcctccagatcaacaacgccttgcttttcaagggaagagcctggaggacggtcgcacactgtctgactataacattcttaaagatcctaaaaagatgccactgctgcgcttgcgt

SEQ ID NO: 949 - DNA, 255 bp, synthetic construct
  source 1..255: mol_type = other DNA
atgcaccaccaccaccaccacggtggatctggcatgttgattttcgtacgcatgttgactggaaagatgatcgagttggaagtggagccttccgatactatcgagaatgttaaggccaaaatccaagatcatgaagggattcctttggatcaacaacgccttgcttttcaagggaagagcctggaggacggtcgcacactgtctgactataacattcttaaagatcctaaaaagatgccactgctgcgcttgcgt

SEQ ID NO: 950 - DNA, 255 bp, synthetic construct
  source 1..255: mol_type = other DNA
atgcaccaccaccaccaccacggtggatctggcatgttgattttcgtacgcatgttgactggaaagatgatcgagttggaagtggagccttccgatactatcgagaatgttaaggccaaaatccaagatcatgaagggattccttgcgatcaacaacgccttgcttttcaagggaagagcctggaggacggtcgcacactgtctgactataacattcttaaagatcctaaaaagatgccactgctgcgcttgcgt

SEQ ID NO: 951 - DNA, 255 bp, synthetic construct
  source 1..255: mol_type = other DNA
atgcaccaccaccaccaccacggtggatctggcatgttgattttcgtacgcatgttgactggaaagatgatcgagttggaagtggagccttccgatactatcgagaatgttaaggccaaaatccaagatcatgaagggattcctccagatgagcaacgccttgcttttcaagggaagagcctggaggacggtcgcacactgtctgactataacattcttaaagatcctaaaaagatgccactgctgcgcttgcgt

SEQ ID NO: 952 - DNA, 255 bp, synthetic construct
  source 1..255: mol_type = other DNA
atgcaccaccaccaccaccacggtggatctggcatgttgattttcgtacgcatgttgactggaaagatgatcgagttggaagtggagccttccgatactatcgagaatgttaaggccaaaatccaagatcatgaagggattcctccagatcaacaacatcttgcttttcaagggaagagcctggaggacggtcgcacactgtctgactataacattcttaaagatcctaaaaagatgccactgctgcgcttgcgt

SEQ ID NO: 953 - DNA, 255 bp, synthetic construct
  source 1..255: mol_type = other DNA
atgcaccaccaccaccaccacggtggatctggcatgttgattttcgtacgcatgttgactggaaagatgatcgagttggaagtggagccttccgatactatcgagaatgttaaggccaaaatccaagatcatgaagggattcctccagatcaacaattccttgcttttcaagggaagagcctggaggacggtcgcacactgtctgactataacattcttaaagatcctaaaaagatgccactgctgcgcttgcgt

SEQ ID NO: 954 - DNA, 255 bp, synthetic construct
  source 1..255: mol_type = other DNA
atgcaccaccaccaccaccacggtggatctggcatgttgattttcgtacgcatgttgactggaaagatgatcgagttggaagtggagccttccgatactatcgagaatgttaaggccaaaatccaagatcatgaagggattcctccagatcaacaacgccttacttttcaagggaagagcctggaggacggtcgcacactgtctgactataacattcttaaagatcctaaaaagatgccactgctgcgcttgcgt

SEQ ID NO: 955 - DNA, 255 bp, synthetic construct
  source 1..255: mol_type = other DNA
atgcaccaccaccaccaccacggtggatctggcatgttgattttcgtacgcatgttgactggaaagatgatcgagttggaagtggagccttccgatactatcgagaatgttaaggccaaaatccaagatcatgaagggattcctccagatcaacaacgccttgcttttcaagggactagcctggaggacggtcgcacactgtctgactataacattcttaaagatcctaaaaagatgccactgctgcgcttgcgt

SEQ ID NO: 956 - DNA, 255 bp, synthetic construct
  source 1..255: mol_type = other DNA
atgcaccaccaccaccaccacggtggatctggcatgttgattttcgtacgcatgttgactggaaagatgatcgagttggaagtggagccttccgatactatcgagaatgttaaggccaaaatccaagatcatgaagggattcctccagatcaacaacgccttgcttttcaagggaagttgctggaggacggtcgcacactgtctgactataacattcttaaagatcctaaaaagatgccactgctgcgcttgcgt

SEQ ID NO: 957 - DNA, 255 bp, synthetic construct
  source 1..255: mol_type = other DNA
atgcaccaccaccaccaccacggtggatctggcatgttgattttcgtacgcatgttgactggaaagatgatcgagttggaagtggagccttccgatactatcgagaatgttaaggccaaaatccaagatcatgaagggattcctccagatcaacaacgccttgcttttcaagggaagatgctggaggacggtcgcacactgtctgactataacattcttaaagatcctaaaaagatgccactgctgcgcttgcgt

SEQ ID NO: 958 - DNA, 255 bp, synthetic construct
  source 1..255: mol_type = other DNA
atgcaccaccaccaccaccacggtggatctggcatgttgattttcgtacgcatgttgactggaaagatgatcgagttggaagtggagccttccgatactatcgagaatgttaaggccaaaatccaagatcatgaagggattcctccagatcaacaacgccttgcttttcaagggaagagcctggatgacggtcgcacactgtctgactataacattcttaaagatcctaaaaagatgccactgctgcgcttgcgt

SEQ ID NO: 959 - DNA, 255 bp, synthetic construct
  source 1..255: mol_type = other DNA
atgcaccaccaccaccaccacggtggatctggcatgttgattttcgtacgcatgttgactggaaagatgatcgagttggaagtggagccttccgatactatcgagaatgttaaggccaaaatccaagatcatgaagggattcctccagatcaacaacgccttgcttttcaagggaagagcctggaggacggttatacactgtctgactataacattcttaaagatcctaaaaagatgccactgctgcgcttgcgt

SEQ ID NO: 960 - DNA, 255 bp, synthetic construct
  source 1..255: mol_type = other DNA
atgcaccaccaccaccaccacggtggatctggcatgttgattttcgtacgcatgttgactggaaagatgatcgagttggaagtggagccttccgatactatcgagaatgttaaggccaaaatccaagatcatgaagggattcctccagatcaacaacgccttgcttttcaagggaagagcctggaggacggtcgcacactgggggactataacattcttaaagatcctaaaaagatgccactgctgcgcttgcgt

SEQ ID NO: 961 - DNA, 255 bp, synthetic construct
  source 1..255: mol_type = other DNA
atgcaccaccaccaccaccacggtggatctggcatgttgattttcgtacgcatgttgactggaaagatgatcgagttggaagtggagccttccgatactatcgagaatgttaaggccaaaatccaagatcatgaagggattcctccagatcaacaacgccttgcttttcaagggaagagcctggaggacggtcgcacactgtctgactataacttgcttaaagatcctaaaaagatgccactgctgcgcttgcgt

SEQ ID NO: 962 - DNA, 255 bp, synthetic construct
  source 1..255: mol_type = other DNA
atgcaccaccaccaccaccacggtggatctggcatgttgattttcgtacgcatgttgactggaaagatgatcgagttggaagtggagccttccgatactatcgagaatgttaaggccaaaatccaagatcatgaagggattcctccagatcaacaacgccttgcttttcaagggaagagcctggaggacggtcgcacactgtctgactataacattcttattgatcctaaaaagatgccactgctgcgcttgcgt

SEQ ID NO: 963 - DNA, 255 bp, synthetic construct
  source 1..255: mol_type = other DNA
atgcaccaccaccaccaccacggtggatctggcatgttgattttcgtacgcatgttgactggaaagatgatcgagttggaagtggagccttccgatactatcgagaatgttaaggccaaaatccaagatcatgaagggattcctccagatcaacaacgccttgcttttcaagggaagagcctggaggacggtcgcacactgtctgactataacattcttaaagatcataaaaagatgccactgctgcgcttgcgt

SEQ ID NO: 964 - DNA, 255 bp, synthetic construct
  source 1..255: mol_type = other DNA
atgcaccaccaccaccaccacggtggatctggcatgttgattttcgtacgcatgttgactggaaagatgatcgagttggaagtggagccttccgatactatcgagaatgttaaggccaaaatccaagatcatgaagggattcctccagatcaacaacgccttgcttttcaagggaagagcctggaggacggtcgcacactgtctgactataacattcttaaagatcctaaaaagatgccactgctgcgcatgcgt

SEQ ID NO: 965 - DNA, 255 bp, synthetic construct
  source 1..255: mol_type = other DNA
atgcaccaccaccaccaccacggtggatctggcatgttgattttcgtacgcatgttgactggaaagatgatcgagttggaagtggagccttccgatactatcgagaatgttaaggccaaaatccaagatcatgaagggattcctccagatcaacaacgccttgcttttcaagggaagagcctggaggacggtcgcacactgtctgactataacattcttaaagatcctaaaaagatgccactgctgcgcttgcaa

SEQ ID NO: 966 - DNA, 255 bp, synthetic construct
  source 1..255: mol_type = other DNA
atgcaccaccaccaccaccacggtggatctggcatgttgattttcgtacgcatgttgactggaaagatgatcgagttggaagtggagccttccgatactatcgagaatgttaaggccaaaatccaagatcatgaagggattcctccagatcaacaacgccttgcttttcaagggaagagcctggaggacggtcgcacactgtctgactataacattcttaaagatcctaaaaagatgttggtactgcgcttgcgt

SEQ ID NO: 967 - DNA, 255 bp, synthetic construct
  source 1..255: mol_type = other DNA
atgcaccaccaccaccaccacggtggatctggcatgttgattttcgtacgcatgttgactggaaagatgatcgagttggaagtgtatccttccgatactatcgagaatgttaaggccaaaatccaagatcatgaagggattcctttggatcaacaacgccttgcttttcaagggaagttgctggaggacggtcgcacactgggggactataacattcttaaagatcctaaaaagatgccactgctgcgcttgcgt

SEQ ID NO: 968 - DNA, 255 bp, synthetic construct
  source 1..255: mol_type = other DNA
atgcaccaccaccaccaccacggtggatctggccatttgattttcgtacgcatgttgactggaaagatgatcgagttggaagtggagccttccgatactatcgagaatgttaaggccaaaatccaagatcatgaagggattcctccagatcaacaacgccttgcttttcaagggaagagcctggaggacggtcgcacactgtctgactataacattcttaaagatcctaaaaagatgccactgctgcgcttgcaa

SEQ ID NO: 969 - DNA, 255 bp, synthetic construct
  source 1..255: mol_type = other DNA
atgcaccaccaccaccaccacggtggatctggctatttgattttcgtacgcatgttgactggaaagatgatcgagttggaagtggagccttccgatactatcgagaatattaaggccaaaatccaagatcatgaagggattcctccagatcaacaacgccttgcttttcaagggaagagcctggaggacggtcgcacactgtctgactataacattcttaaagatcctaaaaagatgccactgctgcgcatgcgt

SEQ ID NO: 970 - DNA, 255 bp, synthetic construct
  source 1..255: mol_type = other DNA
atgcaccaccaccaccaccacggtggatctggcatgttgattttcgtacgcatgttgactggaaagatgatcgagttggaagtggagccttccgatactatcgaggtagttaaggccaaaatccaagatcatgaagggattcctccagatgagcaacgccttgcttttcaagggaagagcctggatgacggtcgcacactgtctgactataacattcttaaagatcctaaaaagatgccactgctgcgcttgcgt

SEQ ID NO: 971 - DNA, 255 bp, synthetic construct
  source 1..255: mol_type = other DNA
atgcaccaccaccaccaccacggtggatctggcatgttgattttcgtacgcatgttgactggaaagatgatcgagttggaagtggagccttccgatactatcgagaatgttaaggccaaaatccaagatcatgaagggattcctccagatcaacaacgccttgcttttcaagggaagagcctggaggacggtcgcacactgtctgactataacttgcttattgatcctaaaaagatgccactgctgcgcttgcgt

SEQ ID NO: 972 - DNA, 255 bp, synthetic construct
  source 1..255: mol_type = other DNA
atgcaccaccaccaccaccacggtggatctggcatgttgattttcgtacgcatgttgactggaaagatgatcgagttggaagtgatgccttccgatactatcgagaatgttaaggccaaaatccaagatcatgaagggattcctccagatcaacaacgccttgcttttcaagggactagcctggatgacggtcgcacactgggggactataacattcttaaagatcctaaaaagatgccactgctgcgcttgcgt

SEQ ID NO: 973 - DNA, 255 bp, synthetic construct
  source 1..255: mol_type = other DNA
atgcaccaccaccaccaccacggtggatctggcatgttgattttcgtacgcatgttgactggaaagatgatcgagttgatggtggagccttccgatactatcgaggtagttaaggccaaaatccaagatcatgaagggattcctccagatgagcaacgccttgcttttcaagggaagttgctggaggacggtcgcacactgtctgactataacattcttaaagatcctaaaaagatgccactgctgcgcttgcgt

SEQ ID NO: 974 - DNA, 255 bp, synthetic construct
  source 1..255: mol_type = other DNA
atgcaccaccaccaccaccacggtggatctggcatgttgattttcgtacgcatgttgactggaaagatgatcgagttggaagtggagccttccgatactatcgagaatattaaggccaaaatccaagatcatgaagggattcctccagatcaatatcgccttgcttttcaagggaagttgctggaggacggtcgcacactgggggactataacattcttaaagatcctaaaaagatgccactgctgcgcttgcgt

SEQ ID NO: 975 - DNA, 255 bp, synthetic construct
  source 1..255: mol_type = other DNA
atgcaccaccaccaccaccacggtggatctggcatgttgattttcgtacgcatgttgactggaaagatgatcgagttggaagtggagccttccgatactatcgagaatgttaaggccaaaatctgcgatcatgaagggattcctccagatcaacaacatcttgcttttcaagggaagagcctggaggacggtcgcacactgggggactataacattcttaaagatcctaaaaagatgccactgctgcgcttgcgt

SEQ ID NO: 976 - DNA, 255 bp, synthetic construct
  source 1..255: mol_type = other DNA
atgcaccaccaccaccaccacggtggatctggcatgttgattttcgtacgcatgttgactggaaagatgatcgagttggaagtgatgccttccgatactatcgaggtagttaaggccaaaatcttcgatcatgaagggattcctccagatcaacaacatcttgcttttcaagggactagcctggaggacggttatacactgggggactataacattcttaaagatcctaaaaagatgccactgctgcgcttgcgt

SEQ ID NO: 977 - DNA, 255 bp, synthetic construct
  source 1..255: mol_type = other DNA
atgcaccaccaccaccaccacggtggatctggcatgttgattttcgtacgcatgttgactggaaagatgatcgagttggaagtgatgccttccgatactatcgaggtagttaaggccaaaatcttcgatcatgaagggattcctccagatcaacaacatcttacttttcaagggactttgctggaggacggttatacactgggggactataacattcttaaagatcctaaaaagatgccactgctgcgcttgcgt

SEQ ID NO: 978 - DNA, 255 bp, synthetic construct
  source 1..255: mol_type = other DNA
atgcaccaccaccaccaccacggtggatctggcatgttgattttcgtacgcatgttgactggaaagatgatcgagttggaagtgatgccttccgatactatcgaggtagttaaggccaaaatcttcgatcatgaagggattcctttggatcaacaacatcttgcttttcaagggactagcctggaggacggttatacactgggggactataacattcttaaagatcctaaaaagatgccactgctgcgcttgcgt

SEQ ID NO: 979 - DNA, 255 bp, synthetic construct
  source 1..255: mol_type = other DNA
atgcaccaccaccaccaccacggtggatctggcatgttgattttcgtacgcatgttgactggaaagatgatcgagttggaagtgatgccttccgatactatcgaggtagttaaggccaaaatccaagatcatgaagggattcctccagatcaacaacatcttgcttttcaagggactagcctggaggacggttatacactgggggactataacattcttaaagatcctaaaaagatgccactgctgcgcttgcgt

SEQ ID NO: 980 - DNA, 255 bp, synthetic construct
  source 1..255: mol_type = other DNA
atgcaccaccaccaccaccacggtggatctggctatttgattttcgtacgcatgttgactggaaagatgatcgagttggaagtggagccttccgatactatcgagaatattaaggccaaaatccaagatcatgaagggattcctccagatcaacaacgccttgcttttcaagggaagagcctggaggacggtcgcacactgtctgactataacttgcttattgatcctaaaaagatgccactgctgcgcatgcgt

SEQ ID NO: 981 - DNA, 255 bp, synthetic construct
  source 1..255: mol_type = other DNA
atgcaccaccaccaccaccacggtggatctggctatttgattttcgtacgcatgttgactggaaagatgatcgagttggaagtggagccttccgatactatcgaggtaattaaggccaaaatccaagatcatgaagggattcctccagatgagcaacgccttgcttttcaagggaagagcctggatgacggtcgcacactgtctgactataacttgcttattgatcctaaaaagatgccactgctgcgcatgcgt

SEQ ID NO: 982 - DNA, 255 bp, synthetic construct
  source 1..255: mol_type = other DNA
atgcaccaccaccaccaccacggtggatctggcatgttgattttcgtacgcatgttgactggaaagatgatcgagttggaagtggagccttccgatactatcgaggtagttaaggccaaaatccaagatcatgaagggattcctccagatgagcaacgccttgcttttcaagggaagagcctggatgacggtcgcacactgtctgactataacttgcttattgatcctaaaaagatgccactgctgcgcttgcgt

SEQ ID NO: 983 - DNA, 255 bp, synthetic construct
  source 1..255: mol_type = other DNA
atgcaccaccaccaccaccacggtggatctggcatgttgattttcgtacgcatgttgactggaaagatgatcgagttggaagtgatgccttccgatactatcgagaatgttaaggccaaaatccaagatcatgaagggattcctccagatcaacaacgccttgcttttcaagggactagcctggatgacggtcgcacactgggggactataacttgcttattgatcctaaaaagatgccactgctgcgcttgcgt

SEQ ID NO: 984 - DNA, 255 bp, synthetic construct
  source 1..255: mol_type = other DNA
atgcaccaccaccaccaccacggtggatctggcatgttgattttcgtacgcatgttgactggaaagatgatcgagttgatggtggagccttccgatactatcgaggtagttaaggccaaaatccaagatcatgaagggattcctccagatgagcaacgccttgcttttcaagggaagttgctggaggacggtcgcacactgtctgactataacttgcttattgatcctaaaaagatgccactgctgcgcttgcgt

SEQ ID NO: 985 - DNA, 255 bp, synthetic construct
  source 1..255: mol_type = other DNA
atgcaccaccaccaccaccacggtggatctggctatttgattttcgtacgcatgttgactggaaagatgatcgagttggaagtgatgccttccgatactatcgagaatattaaggccaaaatccaagatcatgaagggattcctccagatcaacaacgccttgcttttcaagggactagcctggatgacggtcgcacactgggggactataacttgcttattgatcctaaaaagatgccactgctgcgcatgcgt

SEQ ID NO: 986 - DNA, 255 bp, synthetic construct
  source 1..255: mol_type = other DNA
atgcaccaccaccaccaccacggtggatctggctatttgattttcgtacgcatgttgactggaaagatgatcgagttggaagtgatgccttccgatactatcgagaatattaaggccaaaatccaagatcatgaagggattcctccagatcaacaacgccttgcttttcaagggactagcctggatgacggtcgcacactgggggactataacttgcttattgatcctaaaaagatgccactgctgcgcatgcaa

SEQ ID NO: 987 - DNA, 255 bp, synthetic construct
  source 1..255: mol_type = other DNA
atgcaccaccaccaccaccacggtggatctggctatttgattttcgtacgcatgttgactggaaagatgatcgagttggaagtgatgccttccgatactatcgaggtaattaaggccaaaatccaagatcatgaagggattcctccagatgagcaacgccttgcttttcaagggactagcctggatgacggtcgcacactgggggactataacttgcttattgatcctaaaaagatgccactgctgcgcatgcaa

SEQ ID NO: 988 - DNA, 255 bp, synthetic construct
  source 1..255: mol_type = other DNA
atgcaccaccaccaccaccacggtggatctggcatgttgattttcgtacgcatgttgactggaaagatgatcgagttggaagtggagccttccgatactatcgagaatgttaaggccaaaatccaagatcatgaagggattcctccagatcaacaacgccttacttttcaagggaagttgctggaggacggtcgcacactgtctgactataacttgcttattgatcctaaaaagatgccactgctgcgcttgcgt

SEQ ID NO: 989 - DNA, 255 bp, synthetic construct
  source 1..255: mol_type = other DNA
atgcaccaccaccaccaccacggtggatctggcatgttgattttcgtacgcatgttgactggaaagatgatcgagttggaagtggagccttccgatactatcgagaatgttaaggccaaaatccaagatcatgaagggattcctttggatcaacaacgccttacttttcaagggaagttgctggaggacggtcgcacactgtctgactataacttgcttattgatcctaaaaagatgccactgctgcgcttgcgt

SEQ ID NO: 990 - DNA, 255 bp, synthetic construct
  source 1..255: mol_type = other DNA
atgcaccaccaccaccaccacggtggatctggcatgttgattttcgtacgcatgttgactggaaagatgatcgagttggaagtggagccttccgatactatcgagaatgttaaggccaaaatccaagatcatgaagggattcctttggatcaacaacgccttacttttcaagggaagttgctggaggacggtcgcacactgtctgactataacttgcttattgatcctaaaaagatgccactgctgcgcttgcaa

SEQ ID NO: 991 - DNA, 255 bp, synthetic construct
  source 1..255: mol_type = other DNA
atgcaccaccaccaccaccacggtggatctggctatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgccactgctgcgcatgcaa

SEQ ID NO: 992 - DNA, 255 bp, synthetic construct
  source 1..255: mol_type = other DNA
atgcaccaccaccaccaccacggtggatctggcatgttgattttcgtacgcatgttgactggaaagatgatcgagttggaagtggagccttccgatactatcgaggtagttaaggccaaaatccaagatcatgaagggattcctttggatgagcaacgccttacttttcaagggaagttgctggatgacggtcgcacactgtctgactataacttgcttattgatcctaaaaagatgccactgctgcgcttgcaa

SEQ ID NO: 993 - DNA, 255 bp, synthetic construct
  source 1..255: mol_type = other DNA
atgcaccaccaccaccaccacggtggatctggctatttgattttcgtacgcatgttgactggaaagatgatcgagttggaagtggagccttccgatactatcgaggtaattaaggccaaaatccaagatcatgaagggattcctttggatgagcaacgccttacttttcaagggaagttgctggatgacggtcgcacactgtctgactataacttgcttattgatcctaaaaagatgccactgctgcgcatgcaa

SEQ ID NO: 994 - DNA, 255 bp, synthetic construct
  source 1..255: mol_type = other DNA
atgcaccaccaccaccaccacggtggatctggcatgttgattttcgtacgcatgttgactggaaagatgatcgagttggaagtgatgccttccgatactatcgaggtagttaaggccaaaatccaagatcatgaagggattcctccagatcaacaacatcttacttttcaagggactttgctggaggacggttatacactgggggactataacttgcttattgatcctaaaaagatgccactgctgcgcttgcgt

SEQ ID NO: 995 - DNA, 255 bp, synthetic construct
  source 1..255: mol_type = other DNA
atgcaccaccaccaccaccacggtggatctggcatgttgattttcgtacgcatgttgactggaaagatgatcgagttggaagtgatgccttccgatactatcgaggtagttaaggccaaaatccaagatcatgaagggattcctttggatcaacaacatcttacttttcaagggactttgctggaggacggttatacactgggggactataacttgcttattgatcctaaaaagatgccactgctgcgcttgcgt

SEQ ID NO: 996 - DNA, 255 bp, synthetic construct
  source 1..255: mol_type = other DNA
atgcaccaccaccaccaccacggtggatctggcatgttgattttcgtacgcatgttgactggaaagatgatcgagttggaagtgatgccttccgatactatcgaggtagttaaggccaaaatccaagatcatgaagggattcctttggatcaacaacatcttacttttcaagggactttgctggaggacggttatacactgggggactataacttgcttattgatcctaaaaagatgccactgctgcgcttgcaa

SEQ ID NO: 997 - DNA, 255 bp, synthetic construct
  source 1..255: mol_type = other DNA
atgcaccaccaccaccaccacggtggatctggcatgttgattttcgtacgcatgttgactggaaagatgatcgagttggaagtgatgccttccgatactatcgaggtagttaaggccaaaatccaagatcatgaagggattcctttggatgagcaacatcttacttttcaagggactttgctggatgacggttatacactgggggactataacttgcttattgatcctaaaaagatgccactgctgcgcttgcaa

SEQ ID NO: 998 - DNA, 255 bp, synthetic construct
  source 1..255: mol_type = other DNA
atgcaccaccaccaccaccacggtggatctggctatttgattttcgtacgcatgttgactggaaagatgatcgagttggaagtgatgccttccgatactatcgaggtaattaaggccaaaatccaagatcatgaagggattcctttggatgagcaacatcttacttttcaagggactttgctggatgacggttatacactgggggactataacttgcttattgatcctaaaaagatgccactgctgcgcatgcaa

SEQ ID NO: 999 - DNA, 255 bp, synthetic construct
  source 1..255: mol_type = other DNA
atgcaccaccaccaccaccacggtggatctggctatttgattttcgtacgcatgttgactggaaagatgatcgagttggaagtgatgccttccgatactatcgaggtaattaaggccaaaatccaagatcatgaagggattcctccagatgagcaacgccttgcttttcaagggactagcctggatgacggtcgcacactgggggactataacttgcttattgatcctaaaaagatgttggtactgcgcatgcaa

SEQ ID NO: 1000 - DNA, 255 bp, synthetic construct
  source 1..255: mol_type = other DNA
atgcaccaccaccaccaccacggtggatctggctatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgttggtactgcgcatgcaa

SEQ ID NO: 1001 - DNA, 255 bp, synthetic construct
  source 1..255: mol_type = other DNA
atgcaccaccaccaccaccacggtggatctggcatgttgattttcgtacgcatgttgactggaaagatgatcgagttggaagtggagccttccgatactatcgaggtagttaaggccaaaatccaagatcatgaagggattcctttggatgagcaacgccttacttttcaagggaagttgctggatgacggtcgcacactgtctgactataacttgcttattgatcctaaaaagatgttggtactgcgcttgcaa

SEQ ID NO: 1002 - DNA, 255 bp, synthetic construct
  source 1..255: mol_type = other DNA
atgcaccaccaccaccaccacggtggatctggcatgttgattttcgtacgcatgttgactggaaagatgatcgagttggaagtgatgccttccgatactatcgaggtagttaaggccaaaatccaagatcatgaagggattcctttggatcaacaacatcttacttttcaagggactttgctggaggacggttatacactgggggactataacttgcttattgatcctaaaaagatgttggtactgcgcttgcaa

SEQ ID NO: 1003 - DNA, 255 bp, synthetic construct
  source 1..255: mol_type = other DNA
atgcaccaccaccaccaccacggtggatctggctatttgattttcgtacgcatgttgactggaaagatgatcgagttggaagtgatgccttccgatactatcgaggtaattaaggccaaaatccaagatcatgaagggattcctttggatgagcaacatcttacttttcaagggactttgctggatgacggttatacactgggggactataacttgcttattgatcctaaaaagatgttggtactgcgcatgcaa

SEQ ID NO: 1004 - DNA, 255 bp, synthetic construct
  source 1..255: mol_type = other DNA
atgcaccaccaccaccaccacggtggatctggcatgttgattttcgtacgcatgttgactggaaagatgatcgagttggaagtgatgccttccgatactatcgaggtagttaaggccaaaatccaagatcatgaagggattcctccagatcaacaacatcttgcttttcaagggactagcctggaggacggttatacactgggggactataacttgcttattgatcctaaaaagatgttggtactgcgcttgcgt

SEQ ID NO: 1005 - DNA, 255 bp, synthetic construct
  source 1..255: mol_type = other DNA
atgcaccaccaccaccaccacggtggatctggctatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatggccgtactgcgcatgcaa

SEQ ID NO: 1006 - DNA, 255 bp, synthetic construct
  source 1..255: mol_type = other DNA
atgcaccaccaccaccaccacggtggatctggctatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgcgcgtactgcgcatgcaa

SEQ ID NO: 1007 - DNA, 255 bp, synthetic construct
  source 1..255: mol_type = other DNA
atgcaccaccaccaccaccacggtggatctggctatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgaatgtactgcgcatgcaa

SEQ ID NO: 1008 - DNA, 255 bp, synthetic construct
  source 1..255: mol_type = other DNA
atgcaccaccaccaccaccacggtggatctggctatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatggatgtactgcgcatgcaa

SEQ ID NO: 1009 - DNA, 255 bp, synthetic construct
  source 1..255: mol_type = other DNA
atgcaccaccaccaccaccacggtggatctggctatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgtgcgtactgcgcatgcaa

SEQ ID NO: 1010 - DNA, 255 bp, synthetic construct
  source 1..255: mol_type = other DNA
atgcaccaccaccaccaccacggtggatctggctatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatggaggtactgcgcatgcaa

SEQ ID NO: 1011 - DNA, 255 bp, synthetic construct
  source 1..255: mol_type = other DNA
atgcaccaccaccaccaccacggtggatctggctatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgcaagtactgcgcatgcaa

SEQ ID NO: 1012 - DNA, 255 bp, synthetic construct
  source 1..255: mol_type = other DNA
atgcaccaccaccaccaccacggtggatctggctatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgggggtactgcgcatgcaa

SEQ ID NO: 1013 - DNA, 255 bp, synthetic construct
  source 1..255: mol_type = other DNA
atgcaccaccaccaccaccacggtggatctggctatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgcatgtactgcgcatgcaa

SEQ ID NO: 1014 - DNA, 255 bp, synthetic construct
  source 1..255: mol_type = other DNA
atgcaccaccaccaccaccacggtggatctggctatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgattgtactgcgcatgcaa

SEQ ID NO: 1015 - DNA, 255 bp, synthetic construct
  source 1..255: mol_type = other DNA
atgcaccaccaccaccaccacggtggatctggctatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgaaggtactgcgcatgcaa

SEQ ID NO: 1016 - DNA, 255 bp, synthetic construct
  source 1..255: mol_type = other DNA
atgcaccaccaccaccaccacggtggatctggctatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgatggtactgcgcatgcaa

SEQ ID NO: 1017 - DNA, 255 bp, synthetic construct
  source 1..255: mol_type = other DNA
atgcaccaccaccaccaccacggtggatctggctatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgttcgtactgcgcatgcaa

SEQ ID NO: 1018 - DNA, 255 bp, synthetic construct
  source 1..255: mol_type = other DNA
atgcaccaccaccaccaccacggtggatctggctatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgtctgtactgcgcatgcaa

SEQ ID NO: 1019 - DNA, 255 bp, synthetic construct
  source 1..255: mol_type = other DNA
atgcaccaccaccaccaccacggtggatctggctatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgactgtactgcgcatgcaa

SEQ ID NO: 1020 - DNA, 255 bp, synthetic construct
  source 1..255: mol_type = other DNA
atgcaccaccaccaccaccacggtggatctggctatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgtgggtactgcgcatgcaa

SEQ ID NO: 1021 - DNA, 255 bp, synthetic construct
  source 1..255: mol_type = other DNA
atgcaccaccaccaccaccacggtggatctggctatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgtatgtactgcgcatgcaa

SEQ ID NO: 1022 - DNA, 255 bp, synthetic construct
  source 1..255: mol_type = other DNA
atgcaccaccaccaccaccacggtggatctggctatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatggtagtactgcgcatgcaa

SEQ ID NO: 1023 - DNA, 255 bp, synthetic construct
  source 1..255: mol_type = other DNA
atgcaccaccaccaccaccacggtggatctggctatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgttggccctgcgcatgcaa

SEQ ID NO: 1024 - DNA, 255 bp, synthetic construct
  source 1..255: mol_type = other DNA
atgcaccaccaccaccaccacggtggatctggctatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgttgcgcctgcgcatgcaa

SEQ ID NO: 1025 - DNA, 255 bp, synthetic construct
  source 1..255: mol_type = other DNA
atgcaccaccaccaccaccacggtggatctggctatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgttgaatctgcgcatgcaa

SEQ ID NO: 1026 - DNA, 255 bp, synthetic construct
  source 1..255: mol_type = other DNA
atgcaccaccaccaccaccacggtggatctggctatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgttggatctgcgcatgcaa

SEQ ID NO: 1027 - DNA, 255 bp, synthetic construct
  source 1..255: mol_type = other DNA
atgcaccaccaccaccaccacggtggatctggctatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgttgtgcctgcgcatgcaa

SEQ ID NO: 1028 - DNA, 255 bp, synthetic construct
  source 1..255: mol_type = other DNA
atgcaccaccaccaccaccacggtggatctggctatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgttggagctgcgcatgcaa

SEQ ID NO: 1029 - DNA, 255 bp, synthetic construct
  source 1..255: mol_type = other DNA
atgcaccaccaccaccaccacggtggatctggctatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgttgcaactgcgcatgcaa

SEQ ID NO: 1030 - DNA, 255 bp, synthetic construct
  source 1..255: mol_type = other DNA
atgcaccaccaccaccaccacggtggatctggctatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgttggggctgcgcatgcaa

SEQ ID NO: 1031 - DNA, 255 bp, synthetic construct
  source 1..255: mol_type = other DNA
atgcaccaccaccaccaccacggtggatctggctatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgttgcatctgcgcatgcaa

SEQ ID NO: 1032 - DNA, 255 bp, synthetic construct
  source 1..255: mol_type = other DNA
atgcaccaccaccaccaccacggtggatctggctatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgttgattctgcgcatgcaa

SEQ ID NO: 1033 - DNA, 255 bp, synthetic construct
  source 1..255: mol_type = other DNA
atgcaccaccaccaccaccacggtggatctggctatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgttgaagctgcgcatgcaa

SEQ ID NO: 1034 - DNA, 255 bp, synthetic construct
  source 1..255: mol_type = other DNA
atgcaccaccaccaccaccacggtggatctggctatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgttgatgctgcgcatgcaa

SEQ ID NO: 1035 - DNA, 255 bp, synthetic construct
  source 1..255: mol_type = other DNA
atgcaccaccaccaccaccacggtggatctggctatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgttgttcctgcgcatgcaa

SEQ ID NO: 1036 - DNA, 255 bp, synthetic construct
  source 1..255: mol_type = other DNA
atgcaccaccaccaccaccacggtggatctggctatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgttgcctctgcgcatgcaa

SEQ ID NO: 1037 - DNA, 255 bp, synthetic construct
  source 1..255: mol_type = other DNA
atgcaccaccaccaccaccacggtggatctggctatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgttgtctctgcgcatgcaa

SEQ ID NO: 1038 - DNA, 255 bp, synthetic construct
  source 1..255: mol_type = other DNA
atgcaccaccaccaccaccacggtggatctggctatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgttgactctgcgcatgcaa

SEQ ID NO: 1039 - DNA, 255 bp, synthetic construct
  source 1..255: mol_type = other DNA
atgcaccaccaccaccaccacggtggatctggctatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgttgtggctgcgcatgcaa

SEQ ID NO: 1040 - DNA, 255 bp, synthetic construct
  source 1..255: mol_type = other DNA
atgcaccaccaccaccaccacggtggatctggctatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcttattgatcctaaaaagatgttgtatctgcgcatgcaa

SEQ ID NO: 1041 - DNA, 255 bp, synthetic construct
  source 1..255: mol_type = other DNA
atgcaccaccaccaccaccacggtggatctggctatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcctatttctcctgagaagatggccgtactgcgcatgcaa

SEQ ID NO: 1042 - DNA, 255 bp, synthetic construct
  source 1..255: mol_type = other DNA
atgcaccaccaccaccaccacggtggatctggctatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcctatttctcctgagaagatgggggtactgcgcatgcaa

SEQ ID NO: 1043 - DNA, 255 bp, synthetic construct
  source 1..255: mol_type = other DNA
atgcaccaccaccaccaccacggtggatctggctatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcctatttctcctgagaagatgttgatgctgcgcatgcaa

SEQ ID NO: 1044 - DNA, 255 bp, synthetic construct
  source 1..255: mol_type = other DNA
atgcaccaccaccaccaccacggtggatctggctatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcctatttctcctgagaagatggccatgctgcgcatgcaa

SEQ ID NO: 1045 - DNA, 255 bp, synthetic construct
  source 1..255: mol_type = other DNA
atgcaccaccaccaccaccacggtggatctggctatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcctatttctcctgagaagatggccttcctgcgcatgcaa

SEQ ID NO: 1046 - DNA, 255 bp, synthetic construct
  source 1..255: mol_type = other DNA
atgcaccaccaccaccaccacggtggatctggctatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcctatttctcctgagaagatggcctgcctgcgcatgcaa

SEQ ID NO: 1047 - DNA, 255 bp, synthetic construct
  source 1..255: mol_type = other DNA
atgcaccaccaccaccaccacggtggatctggctatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcctatttctcctgagaagatggggatgctgcgcatgcaa

SEQ ID NO: 1048 - DNA, 255 bp, synthetic construct
  source 1..255: mol_type = other DNA
atgcaccaccaccaccaccacggtggatctggctatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcctatttctcctgagaagatggggttcctgcgcatgcaa

SEQ ID NO: 1049 - DNA, 255 bp, synthetic construct
  source 1..255: mol_type = other DNA
atgcaccaccaccaccaccacggtggatctggctatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcctatttctcctgagaagatggggtgcctgcgcatgcaa

SEQ ID NO: 1050 - DNA, 255 bp, synthetic construct
  source 1..255: mol_type = other DNA
atgcaccaccaccaccaccacggtggatctggctatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcctatttctcctgagaagatgtgcatgctgcgcatgcaa

SEQ ID NO: 1051 - DNA, 255 bp, synthetic construct
  source 1..255: mol_type = other DNA
atgcaccaccaccaccaccacggtggatctggctatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcctatttctcctgagaagatgatgatgctgcgcatgcaa

SEQ ID NO: 1052 - DNA, 255 bp, synthetic construct
  source 1..255: mol_type = other DNA
atgcaccaccaccaccaccacggtggatctggctatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcctatttctcctgagaagatgttcatgctgcgcatgcaa

SEQ ID NO: 1053 - DNA, 255 bp, synthetic construct
  source 1..255: mol_type = other DNA
atgcaccaccaccaccaccacggtggatctggctatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttggccatttctcctgagaagatggccgtactgcgcatgcaa

SEQ ID NO: 1054 - DNA, 255 bp, synthetic construct
  source 1..255: mol_type = other DNA
atgcaccaccaccaccaccacggtggatctggctatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcgcatttctcctgagaagatggccgtactgcgcatgcaa

SEQ ID NO: 1055 - DNA, 255 bp, synthetic construct
  source 1..255: mol_type = other DNA
atgcaccaccaccaccaccacggtggatctggctatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgaatatttctcctgagaagatggccgtactgcgcatgcaa

SEQ ID NO: 1056 - DNA, 255 bp, synthetic construct
  source 1..255: mol_type = other DNA
atgcaccaccaccaccaccacggtggatctggctatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttggatatttctcctgagaagatggccgtactgcgcatgcaa

SEQ ID NO: 1057 - DNA, 255 bp, synthetic construct
  source 1..255: mol_type = other DNA
atgcaccaccaccaccaccacggtggatctggctatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgtgcatttctcctgagaagatggccgtactgcgcatgcaa

SEQ ID NO: 1058 - DNA, 255 bp, synthetic construct
  source 1..255: mol_type = other DNA
atgcaccaccaccaccaccacggtggatctggctatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttggagatttctcctgagaagatggccgtactgcgcatgcaa

SEQ ID NO: 1059 - DNA, 255 bp, synthetic construct
  source 1..255: mol_type = other DNA
atgcaccaccaccaccaccacggtggatctggctatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcaaatttctcctgagaagatggccgtactgcgcatgcaa

SEQ ID NO: 1060 - DNA, 255 bp, synthetic construct
  source 1..255: mol_type = other DNA
atgcaccaccaccaccaccacggtggatctggctatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttggggatttctcctgagaagatggccgtactgcgcatgcaa

SEQ ID NO: 1061 - DNA, 255 bp, synthetic construct
  source 1..255: mol_type = other DNA
atgcaccaccaccaccaccacggtggatctggctatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcatatttctcctgagaagatggccgtactgcgcatgcaa

SEQ ID NO: 1062 - DNA, 255 bp, synthetic construct
  source 1..255: mol_type = other DNA
atgcaccaccaccaccaccacggtggatctggctatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgattatttctcctgagaagatggccgtactgcgcatgcaa

SEQ ID NO: 1063 - DNA, 255 bp, synthetic construct
  source 1..255: mol_type = other DNA
atgcaccaccaccaccaccacggtggatctggctatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgaagatttctcctgagaagatggccgtactgcgcatgcaa

SEQ ID NO: 1064 - DNA, 255 bp, synthetic construct
  source 1..255: mol_type = other DNA
atgcaccaccaccaccaccacggtggatctggctatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgatgatttctcctgagaagatggccgtactgcgcatgcaa

SEQ ID NO: 1065 - DNA, 255 bp, synthetic construct
  source 1..255: mol_type = other DNA
atgcaccaccaccaccaccacggtggatctggctatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgttcatttctcctgagaagatggccgtactgcgcatgcaa

SEQ ID NO: 1066 - DNA, 255 bp, synthetic construct
  source 1..255: mol_type = other DNA
atgcaccaccaccaccaccacggtggatctggctatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgtctatttctcctgagaagatggccgtactgcgcatgcaa

SEQ ID NO: 1067 - DNA, 255 bp, synthetic construct
  source 1..255: mol_type = other DNA
atgcaccaccaccaccaccacggtggatctggctatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgactatttctcctgagaagatggccgtactgcgcatgcaa

SEQ ID NO: 1068 - DNA, 255 bp, synthetic construct
  source 1..255: mol_type = other DNA
atgcaccaccaccaccaccacggtggatctggctatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgtggatttctcctgagaagatggccgtactgcgcatgcaa

SEQ ID NO: 1069 - DNA, 255 bp, synthetic construct
  source 1..255: mol_type = other DNA
atgcaccaccaccaccaccacggtggatctggctatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgtatatttctcctgagaagatggccgtactgcgcatgcaa

SEQ ID NO: 1070 - DNA, 255 bp, synthetic construct
  source 1..255: mol_type = other DNA
atgcaccaccaccaccaccacggtggatctggctatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttggtaatttctcctgagaagatggccgtactgcgcatgcaa

SEQ ID NO: 1071 - DNA, 255 bp, synthetic construct
  source 1..255: mol_type = other DNA
atgcaccaccaccaccaccacggtggatctggctatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcctatttctcctgagaagatgtgcttcctgcgcatgcaa

SEQ ID NO: 1072 - DNA, 255 bp, synthetic construct
  source 1..255: mol_type = other DNA
atgcaccaccaccaccaccacggtggatctggctatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcctatttctcctgagaagatgatgttcctgcgcatgcaa

SEQ ID NO: 1073 - DNA, 255 bp, synthetic construct
  source 1..255: mol_type = other DNA
atgcaccaccaccaccaccacggtggatctggctatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcctatttctcctgagaagatgttcttcctgcgcatgcaa

SEQ ID NO: 1074 - DNA, 255 bp, synthetic construct
  source 1..255: mol_type = other DNA
atgcaccaccaccaccaccacggtggatctggctatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcctatttctcctgagaagatgtgctgcctgcgcatgcaa

SEQ ID NO: 1075 - DNA, 255 bp, synthetic construct
  source 1..255: mol_type = other DNA
atgcaccaccaccaccaccacggtggatctggctatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcctatttctcctgagaagatgatgtgcctgcgcatgcaa

SEQ ID NO: 1076 - DNA, 255 bp, synthetic construct
  source 1..255: mol_type = other DNA
atgcaccaccaccaccaccacggtggatctggctatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgcctatttctcctgagaagatgttctgcctgcgcatgcaa

SEQ ID NO: 1077 - DNA, 255 bp, synthetic construct
  source 1..255: mol_type = other DNA
atgcaccaccaccaccaccacggtggatctggctatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttggccatttctcctgagaagatggccatgctgcgcatgcaa

SEQ ID NO: 1078 - DNA, 255 bp, synthetic construct
  source 1..255: mol_type = other DNA
atgcaccaccaccaccaccacggtggatctggctatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgtgcatttctcctgagaagatggccatgctgcgcatgcaa

SEQ ID NO: 1079 - DNA, 255 bp, synthetic construct
  source 1..255: mol_type = other DNA
atgcaccaccaccaccaccacggtggatctggctatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgactatttctcctgagaagatggccatgctgcgcatgcaa

SEQ ID NO: 1080 - DNA, 255 bp, synthetic construct
  source 1..255: mol_type = other DNA
atgcaccaccaccaccaccacggtggatctggctatatgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttggtaatttctcctgagaagatggccatgctgcgcatgcaa

SEQ ID NO: 1081 - DNA, 255 bp, synthetic construct
  source 1..255: mol_type = other DNA
atgcaccatcaccaccaccacggtggatctggctatgccattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgactatttctcctgagaagatggccgtactgcgcatgcaa

SEQ ID NO: 1082 - DNA, 255 bp, synthetic construct
  source 1..255: mol_type = other DNA
atgcaccatcaccaccaccacggtggatctggctatcgcattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgactatttctcctgagaagatggccgtactgcgcatgcaa

SEQ ID NO: 1083 - DNA, 255 bp, synthetic construct
  source 1..255: mol_type = other DNA
atgcaccatcaccaccaccacggtggatctggctataatattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgactatttctcctgagaagatggccgtactgcgcatgcaa

SEQ ID NO: 1084 - DNA, 255 bp, synthetic construct
  source 1..255: mol_type = other DNA
atgcaccatcaccaccaccacggtggatctggctatgatattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgactatttctcctgagaagatggccgtactgcgcatgcaa

SEQ ID NO: 1085 - DNA, 255 bp, synthetic construct
  source 1..255: mol_type = other DNA
atgcaccatcaccaccaccacggtggatctggctattgcattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgactatttctcctgagaagatggccgtactgcgcatgcaa

SEQ ID NO: 1086 - DNA, 255 bp, synthetic construct
  source 1..255: mol_type = other DNA
atgcaccatcaccaccaccacggtggatctggctatgagattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgactatttctcctgagaagatggccgtactgcgcatgcaa

SEQ ID NO: 1087 - DNA, 255 bp, synthetic construct
  source 1..255: mol_type = other DNA
atgcaccatcaccaccaccacggtggatctggctatcaaattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgactatttctcctgagaagatggccgtactgcgcatgcaa

SEQ ID NO: 1088 - DNA, 255 bp, synthetic construct
  source 1..255: mol_type = other DNA
atgcaccatcaccaccaccacggtggatctggctatgggattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgactatttctcctgagaagatggccgtactgcgcatgcaa

SEQ ID NO: 1089 - DNA, 255 bp, synthetic construct
  source 1..255: mol_type = other DNA
atgcaccatcaccaccaccacggtggatctggctatcatattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgactatttctcctgagaagatggccgtactgcgcatgcaa

SEQ ID NO: 1090 - DNA, 255 bp, synthetic construct
  source 1..255: mol_type = other DNA
atgcaccatcaccaccaccacggtggatctggctatattattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgactatttctcctgagaagatggccgtactgcgcatgcaa

SEQ ID NO: 1091 - DNA, 255 bp, synthetic construct
  source 1..255: mol_type = other DNA
atgcaccatcaccaccaccacggtggatctggctataagattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgactatttctcctgagaagatggccgtactgcgcatgcaa

SEQ ID NO: 1092 - DNA, 255 bp, synthetic construct
  source 1..255: mol_type = other DNA
atgcaccatcaccaccaccacggtggatctggctatttgattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgactatttctcctgagaagatggccgtactgcgcatgcaa

SEQ ID NO: 1093 - DNA, 255 bp, synthetic construct
  source 1..255: mol_type = other DNA
atgcaccatcaccaccaccacggtggatctggctatttcattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgactatttctcctgagaagatggccgtactgcgcatgcaa

SEQ ID NO: 1094 - DNA, 255 bp, synthetic construct
  source 1..255: mol_type = other DNA
atgcaccatcaccaccaccacggtggatctggctattctattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgactatttctcctgagaagatggccgtactgcgcatgcaa

SEQ ID NO: 1095 - DNA, 255 bp, synthetic construct
  source 1..255: mol_type = other DNA
atgcaccatcaccaccaccacggtggatctggctatactattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgactatttctcctgagaagatggccgtactgcgcatgcaa

SEQ ID NO: 1096 - DNA, 255 bp, synthetic construct
  source 1..255: mol_type = other DNA
atgcaccatcaccaccaccacggtggatctggctattggattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgactatttctcctgagaagatggccgtactgcgcatgcaa

SEQ ID NO: 1097 - DNA, 255 bp, synthetic construct
  source 1..255: mol_type = other DNA
atgcaccatcaccaccaccacggtggatctggctattatattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgactatttctcctgagaagatggccgtactgcgcatgcaa

SEQ ID NO: 1098 - DNA, 255 bp, synthetic construct
  source 1..255: mol_type = other DNA
atgcaccatcaccaccaccacggtggatctggctatgtaattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgactatttctcctgagaagatggccgtactgcgcatgcaa

SEQ ID NO: 1099 - DNA, 255 bp, synthetic construct
  source 1..255: mol_type = other DNA
atgcaccatcaccaccaccacggtggatctggctatcctattttcgtacgcatgttgactggaaagatgatcgagttggaagtggagccttccgatactatcgagaatattaaggccaaaatccaagatcatgaagggattcctttggatcaacaacgccttacttttcaagggaagttgctggaggacggtcgcacactgtctgactataacttgactatttctcctgagaagatggccgtactgcgcatgcaa

SEQ ID NO: 1100 - protein, 85 aa, synthetic construct
  VAR_SEQ 12: note = X is selected from M, H, Y, W, Q, T, F, S, R, I, and N
  VAR_SEQ 13: note = X is selected from Q, L, I, and M
  VAR_SEQ 17: note = X is selected from K and R
  VAR_SEQ 18: note = X is selected from T, M, I, C, L, and V
  VAR_SEQ 20: note = X is selected from T, I, S, E and V
  VAR_SEQ 23: note = X is selected from T, M, and Y
  VAR_SEQ 24: note = X is selected from I, F, H and P
  VAR_SEQ 25: note = X is selected from T, E, D, H, and N
  VAR_SEQ 27: note = X is selected from E, M, T, N, Y, D, and H
  VAR_SEQ 28: note = X is selected from V and C
  VAR_SEQ 29: note = X is selected from E, M, Y, L, H, F, W, S, Q, T, C, N, R, and D
  VAR_SEQ 30: note = X is selected from P and K
  VAR_SEQ 31: note = X is selected from S, D, N, C, A, and W
  VAR_SEQ 32: note = X is selected from D and E
  VAR_SEQ 36: note = X is selected from N, V, I, E, G, M, Q, D, A, L, R, S, K, T, C, and F
  VAR_SEQ 37: note = X is selected from I, V, and L
  VAR_SEQ 39: note = X is selected from A, E, Q, W, I, M, and D
  VAR_SEQ 40: note = X is selected from K, M, L, R, Q, and H
  VAR_SEQ 42: note = X is selected from Q, C, F, W, H, Y, L, R, and M
  VAR_SEQ 43: note = X is selected from D, A, E, and R
  VAR_SEQ 44: note = X is selected from K, H, A, Q, S, V, L, E, M, T, I, F, C, Y, R, N, and W
  VAR_SEQ 45: note = X is selected from E and T
  VAR_SEQ 49: note = X is selected from P, L, C, F, I, V, Y, T, M, H, S, Q, A, W, N, and K
  VAR_SEQ 50: note = X is selected from D, W, E, G, S, L, and Q
  VAR_SEQ 51: note = X is selected from Q, E, and D
  VAR_SEQ 52: note = X is selected from Q, Y, I, C, and V
  VAR_SEQ 53: note = X is selected from R, W, F, H, Y, N, C, and S
  VAR_SEQ 55: note = X is selected from I, A and T
  VAR_SEQ 57: note = X is selected from A, Q, and G
  VAR_SEQ 59: note = X is selected from K, T, M, I, Q, V, R, L, and N
  VAR_SEQ 60: note = X is selected from Q, S, L, M, P, E V, A, D, I, C, G, and N
  VAR_SEQ 62: note = X is selected from E and D
  VAR_SEQ 63: note = X is selected from D and E
  VAR_SEQ 65: note = X is selected from R, Y, M, T, H, F, N, Q, K, and C
  VAR_SEQ 66: note = X is selected from T and R
  VAR_SEQ 68: note = X is selected from S, G, D, N, H, E, A, Q, M, R, and K
  VAR_SEQ 69: note = X is selected from D and S
  VAR_SEQ 71: note = X is selected from N, E, and Q
  VAR_SEQ 72: note = X is selected from I and L
  VAR_SEQ 73: note = X is selected from Q, L, T, V, C, A, M, I and S
  VAR_SEQ 74: note = X is selected from K, I, M, F, and V
  VAR_SEQ 75: note = X is selected from E, D, and S
  VAR_SEQ 76: note = X is selected from S, P, E, K, H, R, A, D, N, and Q
  VAR_SEQ 77: note = X is selected from T, K, R, and E
  VAR_SEQ 78: note = X is selected from L, H, K, R, S, M, C, Y, and T
  VAR_SEQ 79: note = X is selected from H, M, Q, and E
  VAR_SEQ 80: note = X is selected from L, P, R, A, G, C, F, M, and S
  VAR_SEQ 81: note = X is selected from V, L, M, F, and C
  VAR_SEQ 84: note = X is selected from L and M
  VAR_SEQ 85: note = X is selected from R, Q, V, L, M, C, I, T, E, and K
MHHHHHHGGSGXXIFVXXLXGKXXXLXXXXXXTIEXXKXXIXXXXGIPXXXXXLXFXGXXLXXGXXLXXYXXXXXXXXXXXLRXX

SEQ ID NO: 1101 - DNA, 20 bp, synthetic construct
  source 1..20: mol_type = other DNA
acctctggaaaaaggtaaga

SEQ ID NO: 1102 - RNA, 100 nt, synthetic construct
  source 1..100: mol_type = other RNA
acctctggaaaaaggtaagagttttagagctagaaatagcaagttaaaataaggctagtccgttatcaacttgaaaaagtggcaccgagtcggtgctttt

SEQ ID NO: 1103 - DNA, 86 bp, synthetic construct
  source 1..86: mol_type = other DNA
attccaatgtgataggaactgtaacctctggaaaaaggtagaattcagaggggtgagctttccccttgcctgcccctactgggttt

SEQ ID NO: 1104 - DNA, 20 bp, synthetic construct
  source 1..20: mol_type = other DNA
caaagtcctttcatctgtaa

SEQ ID NO: 1105 - DNA, 86 bp, synthetic construct
  source 1..86: mol_type = other DNA
tgtgtggtgagcgccctgggagccaaagtcctttcatctggaattctaaaggaccggttcatcaacttctttgtaggcaataccat

SEQ ID NO: 1106 - DNA, 20 bp, synthetic construct
  source 1..20: mol_type = other DNA
aattatggggattactagga

SEQ ID NO: 1107 - DNA, 86 bp, synthetic construct
  source 1..86: mol_type = other DNA
aaagactatgaaatggagagctaaattatggggattactagaattcggaaggggcagcaatgagttgacactacagacaaggcact

SEQ ID NO: 1108 - DNA, 20 bp, synthetic construct
  source 1..20: mol_type = other DNA
gaacggatccagctcagcca

SEQ ID NO: 1109 - DNA, 1188 bp, synthetic construct
  source 1..1188: mol_type = other DNA
gtcgtaccgactggtagatgacagcaaacctgttcccttttcggctctgcaacaccgcctagaccgaccggatacacgggtagggcttccgctttacccgtctccctcctggcgcttgtcctcctctcccagtcggcaccacagcggtggctgccgggcgtggtgtcggtgggtcggttggtttttgtctcaccgttggtgtccgtgccgttcagttgcccgccatggctggatctggtggtactagtggaagcaagggtgaggagctgttcaccggagtggtgcctatcctggtcgagctggacggcgacgtaaacggtcacaagttcagcgtgcgtggtgagggcgagggcgatgccaccaacggcaagctgaccctgaagttcatctgcaccactggcaagctgcctgttccatggccaaccctcgtgactacactgacctacggcgttcagtgcttcagccgttaccctgaccatatgaagcgtcacgacttcttcaagtctgccatgcctgaaggctacgtccaggagcgtaccatcagcttcaaggacgatggcacctacaagactcgtgccgaggtgaagttcgagggtgacaccctggtgaaccgcatcgagctgaagggtatcgacttcaaggaggacggcaacatcctgggtcacaagctggagtacaacttcaacagccacaacgtctatatcaccgccgacaagcagaagaacggcatcaaggccaacttcaagattcgtcacaacgtggaggacggtagcgtgcagctcgcagaccactaccagcagaacacgcctatcggcgacggtccagtgttgctgccagacaaccactacctgagcacccagtccgtgctgagcaaagacccgaacgagaagcgcgatcacatggtcctgctggagttcgtgaccgcagccggtatcactggaaccggtgctggaagtggtgagctggatccgttcggcgcccctgccggcgcccctggcggtcccgcgctggggaacggagtggccggcgccggcgaagaagacccggctgcggccttcttggcgcagcaagagagcgagattgcgggcatcgagaacgacgaggccttcgccatcctggacggcggcgcccccgggccccagccgcacggcgagccgccgatccgaaaacgggcgtatagtcgagacc

SEQ ID NO: 1110 - DNA, 140 bp, synthetic construct
  source 1..140: mol_type = other DNA
tttttttttttttttttttttttttttttttttttttttttttttttttttttttttttttttttttttttttttttttttttttttttttttttttttttttttttttttttttttttttagaaggcacagtcgaggct

SEQ ID NO: 1111 - DNA, 25 bp, synthetic construct
  source 1..25: mol_type = other DNA
ccactgcttactggcttatcgaaat

SEQ ID NO: 1112 - DNA, 467 bp, synthetic construct
  source 1..467: mol_type = other DNA
ccactgcttactggcttatcgaaattaatacgactcactatagggagacccaagctggctagcgtttaaacgggccctctagactcgagcggccgccaccatgctgatcttcgtgagaatgctgaccggcaagatgatcgaactggaagtggaacccagcgacaccatcgagaacgtgaaggccaaaatccaggaccacgagggcatccctcctgaccagcagagactggcctttcagggaaagtccctggaagatggaagaaccctgagcgactacaacatcctgaaggaccctaagaagatgccactgctgagactgagatgatcagcctcgactgtgccttctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa